SEQUENCE LISTING (2 sequences)

SEQ ID NO: 1 - protein, 551 aa, Chryseobacterium sp
MDEKNLLEDPDSNLSAGASARALAATPMLHVGGRYLKDPCDNNVVLHGVAITPSPWFNGCQYGANSGYCTWDNYNVQGALNYNKAVMNKLTSAADGWYLNYIRLHIDPYWTNDPGPAIPENDISRFNYNRLVTYTDQVIIPLINHARSLGMYVILRPPGVCPNRIAVNDAYHSYLKTVWTFLSQHPGLKNADNVMFELANEPVEILGTNGTWGSTGNEHFAALKNFFQPLVNIIRNNGANNVCWIPGTGWQSHYQGYVNNQITGGNIGYAVHIYPAYWGGLSNYQAFQNAWNINVKPIADIAPIAITETDWAPQGYGTFGIGTTGTAGGSGFGANLKYIVDQSGNVSWNVLAPDNLLHKGDPNAGTAYNNDWEACAAPVKQWFQQYASSNYPVGNCNTTSSLVNNGIYEIEFQTDANKVVDLKSGEDANGAVLRPWTRNGAAAQRWVAIDAGNGYWRFVSKASATNRCIDLASNSNTLGTSIRLWQNYGNDAQAWQVVAVSNGYYKILSKVDPTRGWDIPNCTMDGNSNLHLWDYYGTSCQLFKFKYIGMN

SEQ ID NO: 2 - DNA, 1656 bp, Chryseobacterium
atggatgaaaaaaatttactagaagaccctgactccaacttaagtgccggcgctagcgcaagagcattggcagcaacaccaatgctgcatgtcggaggcagataccttaaagatccgtgtgacaataatgttgtcttacatggtgtcgccataactccaagcccctggttcaatggctgtcagtatggcgccaattccggctactgtacctgggataattacaatgtacagggcgcactgaactataataaggctgtgatgaacaagctcaccagtgctgctgatggctggtatctcaattacatccgccttcatattgatccgtattggaccaatgatcccggaccggctatcccagagaacgatatctcaagattcaattataaccgcctggtaacttacacagatcaggtgataatcccgctgattaaccatgcccgcagcctgggaatgtatgtcatcctacgtccgccaggcgtatgtccaaatcgtattgctgtgaacgatgcctatcatagctatcttaaaaccgtatggacctttttgtcgcaacatccggggctaaagaacgctgacaatgtgatgttcgaattagccaacgaacctgttgagattctgggtacaaatggcacatggggatcgacaggaaacgagcactttgcagcacttaaaaacttcttccagccattagttaacatcattcgcaacaatggagccaataatgtttgctggataccgggtacaggatggcaatcccattaccaaggctatgtcaataaccagattacaggtggtaatattggttatgctgttcatatctatccggcttactggggcggtctcagcaactatcaagcctttcagaatgcatggaatatcaatgttaaaccaatcgcagacattgcaccaattgccattaccgagaccgactgggcaccgcagggttatggtaccttcggtatcggtacaacaggtacggcaggcggaagcggatttggcgccaatttaaaatatatcgtggatcagtcaggcaatgtaagctggaatgttcttgccccggataatctcctccacaaaggcgatcctaatgcaggaacagcctacaacaacgattgggaagcctgcgccgcaccagttaaacagtggttccagcaatatgcatcctccaattatcctgttggaaactgtaatacaaccagcagcctggttaataatggcatttacgaaatcgagtttcagaccgatgccaataaagtagttgatctaaaatccggagaggatgccaatggcgcagtgttaagaccatggacaaggaatggtgctgctgcacagcgctgggttgcaattgacgccggcaatggttactggcgttttgtatccaaagcgagtgcaaccaatcgctgcattgacttagccagtaacagcaatacactgggaacctcgatcaggctttggcagaactatggtaatgatgcacaagcctggcaggtagttgctgtctccaatggttattataaaatcctgtccaaggtggaccctacacgtggctgggatattcccaactgtaccatggatggcaattcaaacttacacctttgggattattatggtacgtcctgtcagttgttcaagttcaaatatattgggatgaactga

(19) United States
(12) Patent Application Publication
(10) Pub. No.: US 2023/0017381 A1
(43) Pub. Date: Jan. 19, 2023

(54) XYLANASE VARIANTS AND POLYNUCLEOTIDES ENCODING SAME

(71) Applicant: Novozymes A/S, Bagsvaerd (DK)

(72) Inventors: Rakhi Saikia, Bangalore (IN); Amol Vaijanathappa Shivange, Pasadena, CA (US); Sohel Dalal, Juhapura (IN); Kenneth Jensen, Juhapura (DK); Lars Olsen, Valby (DK); Kristian Bertel Romer M. Krogh, Bagsvaerd (DK); Madelyn Mallison Shoup, Raleigh, NC (US); Bernardo Vidal, Jr., Wake Forest, NC (US)

(73) Assignee: Novozymes A/S, Bagsvaerd (DK)

(21) Appl. No.: 17/782,721

(22) PCT Filed: Dec. 17, 2020
(86) PCT No.: PCT/EP2020/086601

§ 371 Date: Jun. 6, 2022

(30) Foreign Application Priority Data
Dec. 19, 2019 (IN) 201941052984

(51) Int. Cl. C12N 9/24; C12P 19/14; C12P 19/04

(52) U.S. Cl. CPC C12N 9/2482; C12Y 302/01008; C12N 9/2402; C12P 19/14; C12P 19/04

(57) ABSTRACT

The present invention relates to xylanase variants of a parent xylanase having increased thermostability when compared to the parent xylanase. The present invention also relates to polynucleotides encoding the variants, nucleic acid constructs, vectors, and host cells comprising the polynucleotides, and method of producing the variants of the present invention.

### REFERENCE TO A SEQUENCE LISTING

This application contains a Sequence Listing in computer readable form, which is incorporated herein by reference.

### FIELD OF THE INVENTION

The present invention relates to polypeptides having xylanase activity and polynucleotides encoding the polypeptides. The invention also relates to nucleic acid constructs, vectors, and host cells comprising the polynucleotides as well as methods of producing and using the polypeptides. The invention also relates to compositions comprising the polypeptides of the invention and the use of the polypeptides of the invention to improve starch and/or gluten yield from corn kernels in a wet milling process.

### BACKGROUND OF THE INVENTION

Conventional wet milling of corn is a process designed for the recovery and purification of starch and several coproducts including germ, gluten (protein) and fiber. Fiber is the least valuable coproduct, so the industry has put substantial effort into increasing the yield of the more valuable products, such as starch and gluten, while decreasing the fiber fraction. High quality starch is valuable as it can be used for a variety of commercial purposes after further processing to products such as dried starch, modified starch, dextrins, sweeteners and alcohol. Gluten is usually used for animal feed, as corn gluten meal (Around 60% protein) or corn gluten feed (Around 20% protein).

The wet milling process can vary significantly dependent on the specific mill equipment used, but usually the process includes grain cleaning, steeping, grinding, germ separation, a second grinding, fiber separation, gluten separation and starch separation. After cleaning the corn kernels, they are typically softened by soaking in water or in a dilute SO₂ solution under controlled conditions of time and temperature. Then, the kernels are grinded to break down the pericarp and the germ is separated from the rest of the kernel. The remaining slurry, mainly consisting of fiber, starch and gluten is finely ground and screened in a fiber washing process, to separate the fiber from starch and gluten, before the gluten and starch is separated and the starch can be purified in a washing/filtration process.

The use of enzymes in several steps of the wet milling process has been suggested, such as the use of enzymes for the steeping step of wet milling processes. The commercial enzyme product Steepzyme® (available from Novozymes A/S) has been shown suitable for the first step in wet milling processes, i.e., the steeping step where corn kernels are soaked in water.

More recently, “enzymatic milling”, a modified wet milling process that uses proteases to significantly reduce the total processing time during corn wet milling and eliminates the need for sulfur dioxide as a processing agent, has been developed. Johnston et al., Cereal Chem, 81, p. 626-632 (2004).

U.S. Pat. No. 6,566,125 discloses a method for obtaining starch from maize involving soaking maize kernels in water to produce soaked maize kernels, grinding the soaked maize kernels to produce a ground maize slurry, and incubating the ground maize slurry with enzyme (e.g., protease).

U.S. Pat. No. 5,066,218 discloses a method of milling grain, especially corn, comprising cleaning the grain, steeping the grain in water to soften it, and then milling the grain with a cellulase enzyme.

WO 2002/000731 discloses a process of treating crop kernels, comprising soaking the kernels in water for 1-12 hours, wet milling the soaked kernels and treating the kernels with one or more enzymes including an acidic protease.

WO 2002/000911 discloses a process of starch gluten separation, comprising subjecting mill starch to an acidic protease.

WO 2002/002644 discloses a process of washing a starch slurry obtained from the starch gluten separation step of a milling process, comprising washing the starch slurry with an aqueous solution comprising an effective amount of acidic protease.

WO 2014/082566 and WO 2014/082564 disclose cellulolytic compositions for use in wet milling.

While the art has investigated the effect of using enzymes in corn wet milling, during steeping/soaking of corn kernels, during grinding of the corn kernels, and in starch gluten separation, there is still a need for improved enzyme technology that may lower the energy expenditure and costs associated with corn wet milling and provide increased yield of starch and gluten.

WO 2016/005522 discloses a GH5 xylanase gene isolated from Chryseobacterium sp-10696 as SEQ ID NO: 25 and the encoded polypeptide as SEQ ID NO: 26.

WO 2019/023222 discloses a method to improve the total starch yield and/or gluten yield that can be obtained from corn kernels in a wet milling process, the method comprising: Admixing corn kernels or a fraction of the corn kernels with an enzyme composition comprising an effective amount of one or more hydrolytic enzymes, wherein at least one of said hydrolytic enzymes is selected from the group consisting of a GH30 polypeptide, a GH5 polypeptide and a combination thereof.

The present invention provides xylanase variants with improved properties compared to its parent.

### SUMMARY OF THE INVENTION

The present invention provides xylanase variant with improved properties compared to parent xylanase.

The present invention relates to a xylanase variants of a parent xylanase comprising a substitution at one or more positions corresponding to positions: 45, 149, 364, 258, 276, and 388 of SEQ ID NO: 1, and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1, and wherein said variant has xylanase activity and wherein the xylanase variant has an increased thermostability compared to the xylanase of SEQ ID NO: 1.

The present invention also relates to a xylanase variants of a parent xylanase, wherein the variant further comprises substitution at one or more positions corresponding to positions: 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1, and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1, and wherein said variant has xylanase activity and wherein the xylanase variant has an increased thermostability compared to the xylanase of SEQ ID NO: 1.

The present invention also relates to a composition comprising the xylanase variants of the invention.

The present invention also relates to a polynucleotide encoding a variant according to the invention, a nucleic acid construct comprising the polynucleotide encoding the variant according to the invention, an expression vector comprising the polynucleotide encoding the variant according to the invention, and a host cell comprising the polynucleotide encoding the variant according to the invention.

The present invention also relates to a method of producing a xylanase variant, comprising (a) cultivating the host cell of the invention under conditions suitable for expression of the variant, and (b) recovering the variant.

The present invention further relates to a method of obtaining a xylanase variant of a parent xylanase comprising the steps of: introducing a substitution at one or more positions corresponding to positions: 45, 149, 364, 258, 276, and 388 of SEQ ID NO: 1, said method thereby providing xylanase variant of said parent xylanase, wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the amino acid sequence to the polypeptide of SEQ ID No: 1, and wherein said variant has xylanase and wherein the xylanase variant has an increased thermostability compared to the xylanase of SEQ ID NO: 1.

The present invention also relates to a method for increasing the starch yield and/or gluten yield from corn kernels in a wet milling process, the method comprising contacting corn kernels or a fraction of the corn kernels, particularly a fiber rich fraction, with an effective amount of xylanase variant of the invention and optionally one or more hydrolytic enzymes.

The present invention also relates to processes of producing fermentation products, such as ethanol, from starch-containing material or cellulosic-containing material, using a fermenting organism.

The present invention also relates to an enzyme blend or composition comprising at least one or more xylanase variant(s) of the present invention.

The present invention also relates to recombinant host cell comprising a heterologous polynucleotide encoding at least one or more xylanase variant(s) of the present invention.

The present invention also relates to a composition (e.g., fermenting or fermented mash composition) comprising: (i) a recombinant host cell or fermenting organism comprising a heterologous polynucleotide encoding an alpha-amylase and/or protease, and at least one or more xylanase variant(s) of the invention.

### Definitions

In accordance with this detailed description, the following definitions apply. Note that the singular forms “a,” “an,” and “the” include plural references unless the context clearly dictates otherwise.

Reference to “about” a value or parameter herein includes aspects that are directed to that value or parameter per se. For example, description referring to “about X” includes the aspect “X”.

Unless defined otherwise or clearly indicated by context, all technical and scientific terms used herein have the same meaning as commonly understood by one of ordinary skill in the art to which this invention belongs.

Allelic variant: The term “allelic variant” means any of two or more alternative forms of a gene occupying the same chromosomal locus. Allelic variation arises naturally through mutation and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Amino acid: The term ‘amino acid’ as used herein, refers to the standard twenty genetically-encoded amino acids and their corresponding stereoisomers in the ‘d’ form (as compared to the natural ‘1’ form), omega-amino acids other naturally-occurring amino acids, unconventional amino acids (e.g. α, α-disubstituted amino acids, N-alkyl amino acids, etc.) and chemically derivatised amino acids. Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

When an amino acid is being specifically enumerated, such as ‘alanine’ or ‘Ala’ or ‘A’, the term refers to both I-alanine and d-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid. In one embodiment, the polypeptides of the invention comprise or consist of I-amino acids.

Arabinofuranosidases/polypeptide with arabinofuranosidase activity: The term “arabinofuranosidase” means an alpha L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,2)- and/or (1,5)-linkages, arabinoxylans, and arabinogalactans. Alpha-L arabinofuranosidase is also known as arabinosidase, alpha-arabinosidase, alpha-L-arabinosidase, alphaarabinofuranosidase, polysaccharide alpha-L-arabinofuranosidase, alpha-L-arabinofuranoside hydrolase, L-arabinosidase, or alpha-L-arabinanase. Arabinofuranosidase activity can be determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, Calif., USA). Arabinofuranosidases can be found in, e.g., the GH43, GH62, GH51 families according to Henrissat, 1991, A classification of glycosyl hydrolases based on amino-acid sequence similarities, Biochem. J. 280: 309-316, and Henrissat and Bairoch, 1996, Updating the sequence-based classification of glycosyl hydrolases, Biochem. J. 316: 695-696.

Beta-glucosidase/polypeptide with beta-glucosidase activity: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase/polypeptide with beta-xylosidase activity: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN® 20.

Cellobiohydrolase/polypeptide with cellobiohydrolase activity: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Contact time: For one or more enzymes to react with a substrate, the one or more enzymes have to be in contact with the substrate. “Contact time” refers to the time period in which an effective amount of one or more enzymes is in contact with at least a fraction of a substrate mass. The enzymes may not be in contact with all of the substrate mass during the contact time, however mixing the one or more enzymes with a substrate mass allows the potential of enzymatically catalyzed hydrolysis of a fraction of the substrate mass during the contact time.

Cellulolytic enzyme or cellulase/polypeptide with cellulase activity or cellulolytic activity: The terms “cellulolytic enzyme”, “cellulase” and polypeptide with cellulase activity or cellulolytic activity are used interchangeably herein and refer to one or more (e.g., several) enzymes that hydrolyze a cellulosic material, which comprise any material comprising cellulose, such as fiber. Cellulolytic enzymes include endoglucanase(s) (E.C 3.2.1.4), cellobiohydrolase(s) (E.C 3.2.1.91 and E.C 3.2.1.150), beta-glucosidase(s) (E.C. 3.2.1.21), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman N21 filter paper, microcrystalline cellulose, bacterial cellulose, algal cellulose, cotton, pretreated lignocellulose, etc. The most common total cellulolytic activity assay is the filter paper assay using Whatman N21 filter paper as the substrate. The assay was established by the International Union of Pure and Applied Chemistry (IUPAC) (Ghose, 1987, Pure Appl. Chem. 59: 257-68).

Cellulolytic enzyme activity can also be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis without addition of cellulolytic enzyme protein. Typical conditions are 1 ml reactions, washed or unwashed PCS, 5% insoluble solids (dry weight), 50 mM sodium acetate 5 pH 5, 1 mM MnSO4, 50° C., 55° C., or 60° C., 72 hours, sugar analysis by AMINEX® HPX-87H column chromatography (Bio-Rad Laboratories, Inc., Hercules, Calif., USA).

Corn kernel: A variety of corn kernels are known, including, e.g., dent corn, flint corn, pod corn, striped maize, sweet corn, waxy corn and the like. Some corn kernels has an outer covering referred to as the “Pericarp” that protects the germ in the kernels. It resists water and water vapour and is undesirable to insects and microorganisms. The only area of the kernels not covered by the “Pericarp” is the “Tip Cap”, which is the attachment point of the kernel to the cob.

Corn kernels or a fraction of the corn kernels: This term is used to describe the corn kernels through the process of wet milling. When the corn kernels are broken down and processed, all fractionated parts of the corn kernel are considered to be included when this term is used. The term include for example: soaked kernels, grinded kernels, corn kernel mass, a first fraction, a second fraction, one or more fractions of the corn kernel mass ect.

Corn kernel mass: is preferably used to reference a mass comprising fiber, gluten and starch, preferably achieved by steaming and grinding crop kernels and separating a mass comprising fiber, gluten and starch from germs. As the corn kernel mass move through the fiber washing, it is separated into several fractions, including a first fraction (s) and a second fraction (f). Hence, “fractions of corn kernel mass” and “one or more fractions of corn kernel mass” refer inter alia to these first (s) and second fractions (f).

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Corresponding to: The term “corresponding to” as used herein, refers to a way of determining the specific amino acid of a sequence wherein reference is made to a specific amino acid sequence. E.g. for the purposes of the present invention, when references are made to specific amino acid positions, the skilled person would be able to align another amino acid sequence to said amino acid sequence that reference has been made to, in order to determine which specific amino acid may be of interest in said another amino acid sequence. Alignment of another amino acid sequence with e.g. the sequence as set forth in SEQ ID NO: 1 or any other sequence listed herein, has been described elsewhere herein. Alternative alignment methods may be used, and are well-known for the skilled person.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Endoglucanase: The term “endoglucanase” means an endo-1,4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Family 61 glycoside hydrolase: The term “Family 61 glycoside hydrolase” or “Family GH61” or “GH61” means a polypeptide falling into the glycoside hydrolase Family 61 according to Henrissat, 1991, A classification of glycosyl hydrolases based on amino-acid sequence similarities, Biochem. J. 280: 309-316, and Henrissat and Bairoch, 1996, Updating the sequence-based classification of glycosyl hydrolases, Biochem. J. 316: 695-696. The enzymes in this family were originally classified as a glycoside hydrolase family based on measurement of very weak endo-1,4-beta-D-glucanase activity in one family member. The structure and mode of action of these enzymes are non-canonical and they cannot be considered as bona fide glycosidases. However, they are kept in the CAZy classification on the basis of their capacity to enhance the breakdown of lignocellulose when used in conjunction with a cellulase or a mixture of cellulases. The GH61 polypeptides have recently been classified as lytic polysaccharide monooxygenases (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061) and are designated “Auxiliary Activity 9” or “AA9” polypeptides.

Fragment: The term “fragment” means a polypeptide having one or more (e.g., several) amino acids absent from the amino and/or carboxyl terminus of a mature polypeptide, wherein the fragment has xylanase activity.

Germ: The “Germ” is the only living part of the corn kernel. It contains the essential genetic information, enzymes, vitamins, and minerals for the kernel to grow into a corn plant. In yellow dent corn, about 25 percent of the germ is corn oil. The endosperm covered or surrounded by the germ comprises about 82 percent of the kernel dry weight and is the source of energy (starch) and protein for the germinating seed. There are two types of endosperm, soft and hard. In the hard endosperm, starch is packed tightly together. In the soft endosperm, the starch is loose.

GH5 polypeptide: refers to a polypeptide with enzyme activity, the polypeptide being classified as member of the Glycoside hydrolase family 5 in the database of Carbohydrate-Active enZYmes (CAZymes) (http://www.cazy.org/).

GH8 polypeptide: refers to a polypeptide with enzyme activity, the polypeptide being classified as member of the Glycoside hydrolase family 8 in the database of Carbohydrate-Active EnZymes (CAZymes) (http://www.cazy.orgn.

GH10 polypeptide: refers to a polypeptide with enzyme activity, the polypeptide being classified as member of the Glycoside hydrolase family 10 in the database of Carbohydrate-Active EnZymes (CAZymes) available at http://www.cazy.org/. (Lombard, V.; Golaconda Ramulu, H.; Drula, E.; Coutinho, P. M.; Henrissat, B. (21 Nov. 2013). “The carbohydrate-active enzymes database (CAZy) in 2013”. Nucleic Acids Research. 42 (Dl): D490-D495; Cantarel B L, Coutinho P M, Rancurel C, Bernard T, Lombard V, Henrissat B (January 2009). “The Carbohydrate-Active EnZymes database (CAZy): an expert resource for Glycogenomics”. Nucleic Acids Res. 37 (Database issue): D233-8).

GH11 polypeptide refers to a polypeptide with enzyme activity, the polypeptide being classified as member of the Glycoside hydrolase family 11 in the database of Carbohydrate-Active EnZymes (CAZymes).

GH30 polypeptide: refers to a polypeptide with enzyme activity, the polypeptide being classified as member of the Glycoside hydrolase family 30 in the database of Carbohydrate-Active enZYmes (CAZymes) (http://www.cazy.org/).

GH62 polypeptide: refers to a polypeptide with enzyme activity, the polypeptide being classified as member of the Glycoside hydrolase family 62 in the database of Carbohydrate-Active EnZymes (CAZymes).

GH43 polypeptide: refers to a polypeptide with enzyme activity, the polypeptide being classified as member of the Glycoside hydrolase family 43 in the database of Carbohydrate-Active EnZymes (CAZymes).

GH51 polypeptide: refers to a polypeptide with enzyme activity, the polypeptide being classified as member of the Glycoside hydrolase family 51 in the database of Carbohydrate-Active EnZymes (CAZymes).

Gluten: Gluten is a protein, made up from two smaller proteins, glutenin and gliadin. Herein “gluten” refers to the majority of proteins found in corn kernels. The major products of gluten from corn wet milling is corn gluten meal (Approximately 60% protein) and corn gluten feed (Approximately 20% protein).

Grind or grinding: The term “grinding” refers to breaking down the corn kernels into smaller components.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Highly branched xylan: The term “highly branched xylan” means that more than 50% of xylosyl units in the arabinoxylan backbone are substituted. This is preferably calculated from linkage analysis as performed in Huismann et al. Carbohydrate Polymers, 2000, 42:269-279.

Hydrolytic enzymes or hydrolase/polypeptide with hydrolase activity: “Hydrolytic enzymes” and polypeptide with hydrolase activity are used interchangeably herein and refer to any catalytic protein that use water to break down substrates. Hydrolytic enzymes include cellulases (EC 3.2.1.4), xylanases (EC 3.2.1.8) arabinofuranosidases (EC 3.2.1.55 (Non-reducing end alpha-L-arabinofuranosidases); EC 3.2.1.185 (Non-reducing end beta-L-arabinofuranosidases) cellobiohydrolase I (EC 3.2.1.150), cellobiohydrolase II (E.C. 3.2.1.91), cellobiosidase (E.C. 3.2.1.176), beta-glucosidase (E.C. 3.2.1.21), beta-xylosidases (EC 3.2.1.37).

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Incubation time: Time in which the one or more fractions of the corn kernel mass is in contact with hydrolytic enzyme during fiber washing, without being screened. In many preferred embodiments, a method according to the present invention utilises a system comprising a space (V), or “incubator”, inside which the material is “left to be affected” by the enzymes and in such situations, the incubation time may be determined by:

$${t}_{i t} = \frac{\begin{matrix}volume of incubator [ {m}^{3} ] * \\ density of inflow to incubator [ kg / {m}^{3} ]\end{matrix}}{mass inflow per time unit to the incubator [ kg / s ]}$$

Alternatively, if the inflow to the incubator is expressed in terms of volume per time unit:

$${t}_{i t} = \frac{volume of i ncubator [ {m}^{3} ]}{volume in flow per time unit to the incubator [ {m}^{3} / s ]}$$

Mill equipment: “Mill equipment” refers to all equipment used on a mill. The wet milling process will vary dependent on the available mill equipment. Examples of mill equipment can be steeping tanks, evaporator, screw press, rotatory dryer, dewatering screen, centrifuge, hydrocyclone ect. The size, and number of each mill equipment/milling lines can vary on different mills, which will affect the milling process. For example, the number of fiber washing screen units can vary and so can the size of a centrifuge.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent xylanase: The term “parent” xylanase as used herein means a xylanase to which modifications are made to produce the variant xylanase of the present invention. This term also refers to the polypeptide with which a variant of the invention is compared. The parent may be a naturally occurring (wild type) polypeptide, or it may even be a variant thereof, prepared by any suitable means. For instance, the parent protein may be a variant of a naturally occurring polypeptide which has been modified or altered in the amino acid sequence. Thus, the parent xylanase may have one or more (or one or several) amino acid substitutions, deletions and/or insertions. Thus, the parent xylanase may be a variant of a parent xylanase. A parent may also be an allelic variant which is a polypeptide encoded by any of two or more alternative forms of a gene occupying the same chromosomal locus. The term “parent” or “parent xylanase” as used herein, refers to the xylanase of SEQ ID NO 1 or any xylanase having at least 60% sequence identity to SEQ ID NO: 1. The parent xylanase may also be a polypeptide comprising a fragment of SEQ ID NO: 1.

Retention time: The time in which one or more hydrolytic enzymes and corn kernels or a fraction of the corn kernels are allowed to react during the fiber washing procedure.

In some embodiments, the retention time is the time period in which the corn kernel mass, received in the first screen unit (51) and one or more fractions thereof, are contacted with an effective amount of one or more hydrolytic enzymes before leaving the fiber washing system again. During the retention time, the one or more fractions of corn kernel mass is incubated with one or more hydrolytic enzymes in a space (V), before it leaves the fiber washing system, as part of a first fraction (s1) from the most upstream screen unit (S1) or as part of a second fraction (f4) from the most downstream screen unit (S4).
Retention time may preferably be estimated as the average duration of time solid matter spends in a fiber washing system as defined in relation to the present invention. This may be estimated by the following relation:

$${t}_{r t} = \frac{Volume of system : [ {m}^{3} ] * density of m as s in flow [ k g / {m}^{3} ]}{mass in flow per t i m e u n i t t o t h e s y s t em [ kg / s ]}$$

Alternatively, if the inflow to the system is expressed in terms of volume per time unit:

$${t}_{i t} = \frac{volume of system [ {m}^{3} ]}{volume in flow per time unit to the system [ {m}^{3} / s ]}$$

The volume of the system is typically set equal to the sum of the volumes of all voids in the system; however, as the tubing in the system typically is made small, it may be preferred to disregard the volume of the tubing.

Screened: The term “screened” or “screening” refers to the process of separating corn kernel mass into a first fraction s and a second fraction f and movement of these fractions from one screen unit to another. A non-screening period is a non-separating period provided for incubation of corn kernel mass or fractions thereof with enzymes.

Sequence identity: The relatedness between two amino acid sequences or between two nucleotide sequences is described by the parameter “sequence identity”. For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. Version 6.1.0 was used.

The optional parameters used are gap open penalty of 10, gap extension penalty of 0.5, and the EBLOSUM62 (EMBOSS version of BLOSUM62) substitution matrix. The output of Needle labelled “longest identity” (obtained using the—nobrief option) is used as the percent identity and is calculated as follows: (Identical Residues×100)/(Length of Alignment—Total Number of Gaps in Alignment).

Starch: The term “starch” means any material comprised of complex polysaccharides of plants, composed of glucose units that occurs widely in plant tissues in the form of storage granules, consisting of amylose and amylopectin, and represented as (C₆H₁₀O₅)n, where n is any number.

Steeping or soaking: The term “steeping” means soaking the crop kernel with water and optionally SO².

Wild-Type Enzyme: The term “wild-type” xylanase denotes a xylanase expressed by a naturally occurring microorganism, such as a bacterium, yeast or filamentous fungus found in nature. The terms “wild-type enzyme” and “parent enzyme” can be used interchangeably when the parent enzyme is not a variant enzyme.

Variant: The term “variant” means a polypeptide having xylanase activity comprising an alteration, i.e., a substitution, insertion, and/or deletion of one or more (several) amino acid residues at one or more (several) positions. A substitution means a replacement of an amino acid occupying a position with a different amino acid; a deletion means removal of an amino acid occupying a position; and an insertion means adding an amino acid adjacent to and immediately following the amino acid occupying a position. The variants of the present invention have at least 20%, e.g., at least 40%, at least 50%, has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the xylanase of the polypeptide of SEQ ID NO: 1.

Xylanases/polypeptide with xylanase activity: The terms “xylanase” and polypeptide with xylanase activity are used interchangeably herein and refer to a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as the amount of xylanase activity that produces 1.0 μmole of azurine per minute at 37° C., pH 6 from 0.2% AZCL-arabinoxylan as substrate in 200 mM sodium phosphate pH 6.

### Nomenclature

For purposes of the present invention, the nomenclature [Y/F] means that the amino acid at this position may be a tyrosine (Try, Y) or a phenylalanine (Phe, F). Likewise, the nomenclature [V/G/A/I] means that the amino acid at this position may be a valine (Val, V), glycine (Gly, G), alanine (Ala, A) or isoleucine (Ile, I), and so forth for other combinations as described herein. The amino acid X is defined such that it may be any of the 20 natural amino acids, unless otherwise stated.

### Conventions for Designation of Variants

For purposes of the present invention, SEQ ID NO: 1 is used to determine the corresponding amino acid residue in another xylanase. The amino acid sequence of another xylanase is aligned with SEQ ID NO: 1, and based on the alignment, the amino acid position number corresponding to any amino acid residue in SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), e.g., version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another xylanase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1794), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other enzyme has diverged from the polypeptide of SEQ ID NO: 1 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example, the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.
Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.
Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”. In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by a plus sign (“+”), e.g., “Arg170Tyr+Gly195Glu” or “R170Y+G195E” representing a substitution of arginine and glycine at positions 170 and 195 with tyrosine and glutamic acid, respectively.
Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates to xylanase variants of a parent xylanase comprising a substitution at one or more positions corresponding to positions: 45, 149, 364, 258, 276, and 388 of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1, and wherein said variant has xylanase activity.

The present invention relates to xylanase variants of a parent xylanase further comprises a substitution at one or more positions corresponding to positions: 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1, and wherein said variant has xylanase activity.

In one aspect, present invention relates to xylanase variants comprising a substitution at one or more positions corresponding to positions: 45, 149, 364, 258, 276, and 388 of SEQ ID NO: 1; and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1, and wherein said variant has xylanase activity and wherein the xylanase variant has an improved property compared to the xylanase of SEQ ID NO: 1.

In one aspect, present invention relates to xylanase variants further comprises a substitution at one or more positions corresponding to positions: 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1; and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1, and wherein said variant has xylanase activity and wherein the xylanase variant has an improved property relative to said parent.

The invention relates to xylanase variants having one or more substitutions having an improved property, such as improved (increased) thermostability.

In one aspect, present invention relates to xylanase variants comprising a substitution at one or more positions corresponding to positions: 45, 149, 364, 258, 276, and 388 of SEQ ID NO: 1; and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1, and wherein said variant has xylanase activity and wherein the xylanase variant has increased thermostability compared to the xylanase of SEQ ID NO: 1.

In one aspect, present invention relates to xylanase variants further comprises a substitution at one or more positions corresponding to positions: 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1; and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1, and wherein said variant has xylanase activity and wherein the xylanase variant has increased thermostability compared to the xylanase of SEQ ID NO: 1.

### Xylanase Variants

The present invention provides xylanase variants of a parent xylanase comprising a substitution at one or more positions corresponding to position 45, 149, 364, 258, 276, and 388 of SEQ ID NO: 1 and wherein said variant has increased thermostability compared to the xylanase of SEQ ID NO: 1.

The present invention also provides xylanase variants of a parent xylanase further comprising a substitution at one or more positions corresponding to position 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1 and wherein said variant has increased thermostability compared to the xylanase of SEQ ID NO: 1.

In one aspect, the variant has sequence identity of at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% to the amino acid sequence of the parent alpha-amylase.

In one aspect, the variant has a sequence identity of at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% to SEQ ID NO: 1.

In one aspect, the substituted amino acid residue is different from the naturally-occurring amino acid residue in that position. In one embodiment, the substitution is selected from the group consisting of A, C, D, E, F, G, H, I, K, L, M, N, P, Q, R, S, T, V, W and Y, with the proviso that the substituted amino acid residue is different from the naturally-occurring amino acid residue in that position.

In one aspect, the number of substitutions is 1-50, e.g., 1-45, 1-40, 1-35, 1-30, 1-25, 1-20, 1-15, 1-10 or 1-5, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 substitutions.

In another aspect, a variant comprises a substitution at two or more positions corresponding to any of positions 45, 149, 364, 258, 276, and 388 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at three or more positions corresponding to any of positions 45, 149, 364, 258, 276, and 388 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at four or more positions corresponding to any of positions 45, 149, 364, 258, 276, and 388 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at five or more positions corresponding to any of positions 45, 149, 364, 258, 276, and 388 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at six or more positions corresponding to any of positions 45, 149, 364, 258, 276, and 388 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at each position corresponding to any of positions 45, 149, 364, 258, 276, and 388 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at two or more positions corresponding to any of positions 45, 149, 364, 258, 276, 388, 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at three or more positions corresponding to any of positions 45, 149, 364, 258, 276, 388, 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at four or more positions corresponding to any of positions 45, 149, 364, 258, 276, 388, 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at five or more positions corresponding to any of positions 45, 149, 364, 258, 276, 388, 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at six or more positions corresponding to any of positions 45, 149, 364, 258, 276, 388, 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at seven or more positions corresponding to any of positions 45, 149, 364, 258, 276, 388, 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at eight or more positions corresponding to any of positions 45, 149, 364, 258, 276, 388, 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at nine or more positions corresponding to any of positions 45, 149, 364, 258, 276, 388, 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at ten or more positions corresponding to any of positions 45, 149, 364, 258, 276, 388, 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1 and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In another aspect, a variant comprises a substitution at each position corresponding to positions 45, 149, 364, 258, 276, 388, 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1 and wherein said variant has xylanase activity and wherein said variant has xylanase activity and further wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

In one aspect, a variant comprises one or more of the following substitutions at positions corresponding to positions: D9, D11, A18, S19, A20, A24, A25, M28, C40, V45, C60, Q61, C69, T70, Q77, A79, S92, A93, D95, D113, A117, N127, L149, C161, N163, N168, A170, S173, T180, G187, S190, D192, T211, S214, T215, H219, V234, W250, A252, V258, N260, A276, L281, S282, A286, Q314, H334, A340, V350, N355, L356, H358, A364, T366, C375, Q384, Q385, Y386, S388, S389, Y391, V393, G394, N395, C396, D446, R459, C468, 1469, D470, L471, A472, A472, S473, S473, N474, N476, T477, L478, T480, S481, R483, L484, W495, Q496, V497, V498, A499, V500, S501, G503, C522, D525, G526, N527, S528, N529, L530 and C540 of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises one or more of the following substitutions at positions corresponding to positions D9E, D11E, A18V, 519T, A20H, A20P, A24H, A24K, A25W, M28P, C40A, C40L, C40S, C40V, V45C, V45I, V45L, V45N, V45T, C60A, C60L, C60S, C60V, Q61K, Q61R, C69A, C69L, C69S, C69V, T70R, Q77E, A79C, S92D, S92N, A93S, A93T, D95N, D113T, A117C, A117S, N127D, L149R, C161L, C161S, C161V, N163H, N168G, A170S, S173Y, T180Q, G187K, G187L, G187S, S190E, D192E, T211H, S214E, S214Q, S214Y, T215I, T215K, T215V, H219I, H219V, V234P, C243L, C243S, C243V, W250Y, A252S, V258P, V258R, N260H, A276C, A276D, A276F, A276N, A276Q, A276S, A276T, L281V, S282N, A286E, A286S, Q314A, H334D, A340G, V350L, N355G, L356M, H358D, H358T, A364G, T366I, T366V, C375A, C375L, C375S, C375V, Q384A, Q385C, Y386G, 5388A, S388H, S388K, S388L, 5388R, S389H, S389V, Y391V, V393H, V393R, G394A, G394D, G394Q, N395D, C396A, C396L, C396A, C396S, C396V, D446T, R459V, C468A, C468L, C468S, C468V, 1469T, D470A, D470K, D470T, D470V, L471R, A472I, A472L, S473G, S473K, N474G, N476D, T477K, T477V, L478N, L478P, T480L, T480N, S481A, R483V, L484D, L484K, L484S, W495E, W495I, W495S, Q496E, Q496W, Q496R, V497D, V497E, V497P, V497W, V498I, A499L, A499Y, V500T, S501E, 5501N, G503L, C522A, C522S, C522V, D525L, G526T, N527H, S528H, N529T, L530R, C540A, C540L, C540S, and C540V of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In another aspect, the variant comprises or consists of a substitution at a position corresponding to position 9 of SEQ ID NO: 1. In another aspect, the amino acid at a position corresponding to position 9 is substituted with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of the substitution D9E of the polypeptide of SEQ ID NO: 1.

In another aspect, the variant comprises or consists of a substitution at a position corresponding to position 11 of SEQ ID NO: 1. In another aspect, the amino acid at a position corresponding to position 3 is substituted with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of the substitution D11E of the polypeptide of SEQ ID NO: 1.

In another aspect, the variant comprises or consists of a substitution at a position corresponding to position 18 of SEQ ID NO: 1. In another aspect, the amino acid at a position corresponding to position 18 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A18V of the polypeptide of SEQ ID NO: 1.

In another aspect, the variant comprises or consists of a substitution at a position corresponding to position 19 of SEQ ID NO: 1. In another aspect, the amino acid at a position corresponding to position 19 is substituted with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of the substitution 519T of the polypeptide of SEQ ID NO: 1.

In another aspect, the variant comprises or consists of a substitution at a position corresponding to position 20 of SEQ ID NO: 1. In another aspect, the amino acid at a position corresponding to position 20 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A20H or A20P of the polypeptide of SEQ ID NO: 1.

In another aspect, the variant comprises or consists of a substitution at a position corresponding to position 24 of SEQ ID NO: 1. In another aspect, the amino acid at a position corresponding to position 24 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A24H or A24K of the polypeptide of SEQ ID NO: 1.

In another aspect, the variant comprises or consists of a substitution at a position corresponding to position 25 of SEQ ID NO: 1. In another aspect, the amino acid at a position corresponding to position 25 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A25W of the polypeptide of SEQ ID NO: 1.

In another aspect, the variant comprises or consists of a substitution at a position corresponding to position 28 of SEQ ID NO: 1. In another aspect, the amino acid at a position corresponding to position 28 is substituted with Ala, Arg, Asn, Asp, Cys, Gln, Glu, Gly, His, Ile, Leu, Lys, Phe, Pro, Ser, Thr, Trp, Tyr, or Val. In another aspect, the variant comprises or consists of the substitution M28P of the polypeptide of SEQ ID NO: 1.

In another aspect, the variant comprises or consists of a substitution at a position corresponding to position 40 of SEQ ID NO: 1. In another aspect, the amino acid at a position corresponding to position 40 is substituted with Ala, Arg, Asn, Asp, Gln, Glu, Gly, His, Ile, Leu, Lys, Met, Phe, Pro, Ser, Thr, Trp, Tyr, or Val. In another aspect, the variant comprises or consists of the substitution C40A or C40L or C40S or C40V of the polypeptide of SEQ ID NO: 1.

In another aspect, the variant comprises or consists of a substitution at a position corresponding to position 45 of SEQ ID NO: 1. In another aspect, the amino acid at a position corresponding to position 45 is substituted with Ala, Arg, Asn, Asp, Cys, Gln, Glu, Gly, His, Ile, Leu, Lys, Met, Phe, Pro, Ser, Thr, Trp, or Tyr. In another aspect, the variant comprises or consists of the substitution V45C or V45I or V45L, V45N or V45T of the polypeptide of SEQ ID NO: 1.

In another aspect, the variant comprises or consists of a substitution at a position corresponding to position 60 of SEQ ID NO: 1. In another aspect, the amino acid at a position corresponding to position 60 is substituted with Ala, Arg, Asn, Asp, Gin, Glu, Gly, His, Ile, Leu, Lys, Met, Phe, Pro, Ser, Thr, Trp, Tyr, or Val. In another aspect, the variant comprises or consists of the substitution C60A or C60L or C60S or C60V of the polypeptide of SEQ ID NO: 1.

In another aspect, the variant comprises or consists of a substitution at a position corresponding to position 61 of SEQ ID NO: 1. In another aspect, the amino acid at a position corresponding to position 61 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q61K or Q61R of the polypeptide of SEQ ID NO: 1.

In another aspect, the variant comprises or consists of a substitution at a position corresponding to position 69 of SEQ ID NO: 1. In another aspect, the amino acid at a position corresponding to position 69 is substituted with Ala, Arg, Asn, Asp, Gin, Glu, Gly, His, Ile, Leu, Lys, Met, Phe, Pro, Ser, Thr, Trp, Tyr, or Val. In another aspect, the variant comprises or consists of the substitution C69A or C69L or C69S or C69V of the polypeptide of SEQ ID NO: 1.

In another aspect, the variant comprises or consists of a substitution at a position corresponding to position 70 of SEQ ID NO: 1. In another aspect, the amino acid at a position corresponding to position 70 is substituted with Ala, Arg, Asn, Asp, Cys, Gin, Glu, Gly, His, Ile, Leu, Lys, Met, Phe, Pro, Ser, Trp, Tyr, or Val. In another aspect, the variant comprises or consists of the substitution T70R of the polypeptide of SEQ ID NO: 1.

In another aspect, the variant comprises or consists of a substitution at a position corresponding to position 77 of SEQ ID NO: 1. In another aspect, the amino acid at a position corresponding to position 77 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q77E of the polypeptide of SEQ ID NO: 1.

In another aspect, the variant comprises or consists of a substitution at a position corresponding to position 79 of SEQ ID NO: 1. In another aspect, the amino acid at a position corresponding to position 79 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A79C of the polypeptide of SEQ ID NO: 1.

In another aspect, the variant comprises or consists of a substitution at a position corresponding to position 92 of SEQ ID NO: 1. In another aspect, the amino acid at a position corresponding to position 92 is substituted with Ala, Arg, Asn, Asp, Cys, Gin, Glu, Gly, His, Ile, Leu, Lys, Met, Phe, Pro, Thr, Trp, Tyr, or Val. In another aspect, the variant comprises or consists of the substitution S92D or S92N of the polypeptide of SEQ ID NO: 1.

In another aspect, the variant comprises or consists of a substitution at a position corresponding to position 93 of SEQ ID NO: 1. In another aspect, the amino acid at a position corresponding to position 93 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A93S or A93T of the polypeptide of SEQ ID NO: 1.

In another aspect, the variant comprises or consists of a substitution at a position corresponding to position 95 of SEQ ID NO: 1. In another aspect, the amino acid at a position corresponding to position 95 is substituted with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of the substitution D95N of the polypeptide of SEQ ID NO: 1.

In another aspect, the variant comprises or consists of a substitution at a position corresponding to position 113 of SEQ ID NO: 1. In another aspect, the amino acid at a position corresponding to position 113 is substituted with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of the substitution D113T of the polypeptide of SEQ ID NO: 1.

In another aspect, the variant comprises or consists of a substitution at a position corresponding to position 117 of SEQ ID NO: 1. In another aspect, the amino acid at a position corresponding to position 117 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A117C or A117S of the polypeptide of SEQ ID NO: 1.

In another aspect, the variant comprises or consists of a substitution at a position corresponding to position 127 of SEQ ID NO: 1. In another aspect, the amino acid at a position corresponding to position 127 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N127D of the polypeptide of SEQ ID NO: 1.

In another aspect, the variant comprises or consists of a substitution at a position corresponding to position 149 of SEQ ID NO: 1. In another aspect, the amino acid at a position corresponding to position 149 is substituted with Ala, Arg, Asn, Asp, Cys, Gln, Glu, Gly, His, Ile, Lys, Met, Phe, Pro, Ser, Thr, Trp, Tyr, or Val. In another aspect, the variant comprises or consists of the substitution L149R of the polypeptide of SEQ ID NO: 1.

In another aspect, the variant comprises or consists of a substitution at a position corresponding to position 161 of SEQ ID NO: 1. In another aspect, the amino acid at a position corresponding to position 161 is substituted with Ala, Arg, Asn, Asp, Gln, Glu, Gly, His, Ile, Leu, Lys, Met, Phe, Pro, Ser, Thr, Trp, Tyr, or Val. In another aspect, the variant comprises or consists of the substitution C161L or C161S or C161V of the polypeptide of SEQ ID NO: 1.

In another aspect, the variant comprises or consists of a substitution at a position corresponding to position 163 of SEQ ID NO: 1. In another aspect, the amino acid at a position corresponding to position 163 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N163H of the polypeptide of SEQ ID NO: 1.

In another aspect, the variant comprises or consists of a substitution at a position corresponding to position 168 of SEQ ID NO: 1. In another aspect, the amino acid at a position corresponding to position 168 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N168G of the polypeptide of SEQ ID NO: 1.

In another aspect, the variant comprises or consists of a substitution at a position corresponding to position 170 of SEQ ID NO: 1. In another aspect, the amino acid at a position corresponding to position 170 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A1705 of the polypeptide of SEQ ID NO: 1.

In another aspect, the variant comprises or consists of a substitution at a position corresponding to position 173 of SEQ ID NO: 1. In another aspect, the amino acid at a position corresponding to position 173 is substituted with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of the substitution S173Y of the polypeptide of SEQ ID NO: 1.

In another aspect, the variant comprises or consists of a substitution at a position corresponding to position 180 of SEQ ID NO: 1. In another aspect, the amino acid at a position corresponding to position 180 is substituted with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of the substitution T180Q of the polypeptide of SEQ ID NO: 1.

In another aspect, the variant comprises or consists of a substitution at a position corresponding to position 187 of SEQ ID NO: 1. In another aspect, the amino acid at a position corresponding to position 187 is substituted with Ala, Arg, Asn, Asp, Cys, Gln, Glu, His, Ile, Leu, Lys, Met, Phe, Pro, Ser, Thr, Trp, Tyr, or Val. In another aspect, the variant comprises or consists of the substitution G187K or G187L or G187S of the polypeptide of SEQ ID NO: 1.

In another aspect, the variant comprises or consists of a substitution at a position corresponding to position 190 of SEQ ID NO: 1. In another aspect, the amino acid at a position corresponding to position 190 is substituted with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of the substitution 5190E of the polypeptide of SEQ ID NO: 1.

In another aspect, the variant comprises or consists of a substitution at a position corresponding to position 192 of SEQ ID NO: 1. In another aspect, the amino acid at a position corresponding to position 192 is substituted with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of the substitution D192E of the polypeptide of SEQ ID NO: 1.

In another aspect, the variant comprises or consists of a substitution at a position corresponding to position 211 of SEQ ID NO: 1. In another aspect, the amino acid at a position corresponding to position 211 is substituted with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of the substitution T211H of the polypeptide of SEQ ID NO: 1.

In another aspect, the variant comprises or consists of a substitution at a position corresponding to position 214 of SEQ ID NO: 1. In another aspect, the amino acid at a position corresponding to position 214 is substituted with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of the substitution S214E or S214Q or S214Y of the polypeptide of SEQ ID NO: 1.

In another aspect, the variant comprises or consists of a substitution at a position corresponding to position 215 of SEQ ID NO: 1. In another aspect, the amino acid at a position corresponding to position 215 is substituted with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of the substitution T215I or T215K or T215V of the polypeptide of SEQ ID NO: 1.

In another aspect, the variant comprises or consists of a substitution at a position corresponding to position 219 of SEQ ID NO: 1. In another aspect, the amino acid at a position corresponding to position 219 is substituted with Ala, Arg, Asn, Asp, Cys, Gin, Glu, Gly, Ile, Leu, Lys, Met, Phe, Pro, Ser, Thr, Trp, Tyr, or Val. In another aspect, the variant comprises or consists of the substitution H219I or H219V of the polypeptide of SEQ ID NO: 1.

In another aspect, the variant comprises or consists of a substitution at a position corresponding to position 234 of SEQ ID NO: 1. In another aspect, the amino acid at a position corresponding to position 234 is substituted with Ala, Arg, Asn, Asp, Cys, Gin, Glu, Gly, His, Ile, Leu, Lys, Met, Phe, Pro, Ser, Thr, Trp, or Tyr. In another aspect, the variant comprises or consists of the substitution V234P of the polypeptide of SEQ ID NO: 1.

In another aspect, the variant comprises or consists of a substitution at a position corresponding to position 243 of SEQ ID NO: 1. In another aspect, the amino acid at a position corresponding to position 243 is substituted with Ala, Arg, Asn, Asp, Gln, Glu, Gly, His, Ile, Leu, Lys, Met, Phe, Pro, Ser, Thr, Trp, Tyr, or Val. In another aspect, the variant comprises or consists of the substitution C243L or C243S or C243V of the polypeptide of SEQ ID NO: 1.

In another aspect, the variant comprises or consists of a substitution at a position corresponding to position 250 of SEQ ID NO: 1. In another aspect, the amino acid at a position corresponding to position 250 is substituted with Ala, Arg, Asn, Asp, Cys, Gln, Glu, Gly, His, Ile, Leu, Lys, Met, Phe, Pro, Ser, Thr, Tyr, or Val. In another aspect, the variant comprises or consists of the substitution W250Y of the polypeptide of SEQ ID NO: 1.

In another aspect, the variant comprises or consists of a substitution at a position corresponding to position 252 of SEQ ID NO: 1. In another aspect, the amino acid at a position corresponding to position 252 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A252S of the polypeptide of SEQ ID NO: 1.

In another aspect, the variant comprises or consists of a substitution at a position corresponding to position 258 of SEQ ID NO: 1. In another aspect, the amino acid at a position corresponding to position 258 is substituted with Ala, Arg, Asn, Asp, Cys, Gln, Glu, Gly, His, Ile, Leu, Lys, Met, Phe, Pro, Ser, Thr, Trp, or Tyr. In another aspect, the variant comprises or consists of the substitution V258P or V258R of the polypeptide of SEQ ID NO: 1.

In another aspect, the variant comprises or consists of a substitution at a position corresponding to position 260 of SEQ ID NO: 1. In another aspect, the amino acid at a position corresponding to position 260 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N260H of the polypeptide of SEQ ID NO: 1.

In another aspect, the variant comprises or consists of a substitution at a position corresponding to position 276 of SEQ ID NO: 1. In another aspect, the amino acid at a position corresponding to position 276 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A276C or A276D or A276F or A276N or A276Q or A276S or A276T of the polypeptide of SEQ ID NO: 1.

In another aspect, the variant comprises or consists of a substitution at a position corresponding to position 281 of SEQ ID NO: 1. In another aspect, the amino acid at a position corresponding to position 281 is substituted with Ala, Arg, Asn, Asp, Cys, Gln, Glu, Gly, His, Ile, Lys, Met, Phe, Pro, Ser, Thr, Trp, Tyr, or Val. In another aspect, the variant comprises or consists of the substitution L281V of the polypeptide of SEQ ID NO: 1.

In another aspect, the variant comprises or consists of a substitution at a position corresponding to position 282 of SEQ ID NO: 1. In another aspect, the amino acid at a position corresponding to position 282 is substituted with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of the substitution S282N of the polypeptide of SEQ ID NO: 1.

In another aspect, the variant comprises or consists of a substitution at a position corresponding to position 286 of SEQ ID NO: 1. In another aspect, the amino acid at a position corresponding to position 286 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A286E or A286S of the polypeptide of SEQ ID NO: 1.

In another aspect, the variant comprises or consists of a substitution at a position corresponding to position 314 of SEQ ID NO: 1. In another aspect, the amino acid at a position corresponding to position 314 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q314A of the polypeptide of SEQ ID NO: 1.

In another aspect, the variant comprises or consists of a substitution at a position corresponding to position 334 of SEQ ID NO: 1. In another aspect, the amino acid at a position corresponding to position 334 is substituted with Ala, Arg, Asn, Asp, Cys, Gin, Glu, Gly, Ile, Leu, Lys, Met, Phe, Pro, Ser, Thr, Trp, Tyr, or Val. In another aspect, the variant comprises or consists of the substitution H334D of the polypeptide of SEQ ID NO: 1.

In another aspect, the variant comprises or consists of a substitution at a position corresponding to position 340 of SEQ ID NO: 1. In another aspect, the amino acid at a position corresponding to position 340 is substituted with Arg, Asn, Asp, Cys, Gln, Glu, Gly, Ile, Leu, Lys, Met, His, Phe, Pro, Ser, Thr, Trp, Tyr, or Val. In another aspect, the variant comprises or consists of the substitution A340G of the polypeptide of SEQ ID NO: 1.

In another aspect, the variant comprises or consists of a substitution at a position corresponding to position 350 of SEQ ID NO: 1. In another aspect, the amino acid at a position corresponding to position 3 is substituted with Ala, Arg, Asn, Asp, Cys, Gln, Glu, Gly, His, Ile, Leu, Lys, Met, Phe, Pro, Ser, Thr, Trp, or Tyr. In another aspect, the variant comprises or consists of the substitution V350L of the polypeptide of SEQ ID NO: 1.

In another aspect, the variant comprises or consists of a substitution at a position corresponding to position 355 of SEQ ID NO: 1. In another aspect, the amino acid at a position corresponding to position 355 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N355G of the polypeptide of SEQ ID NO: 1.

In another aspect, the variant comprises or consists of a substitution at a position corresponding to position 356 of SEQ ID NO: 1. In another aspect, the amino acid at a position corresponding to position 356 is substituted with Ala, Arg, Asn, Asp, Cys, Gln, Glu, Gly, His, Ile, Lys, Met, Phe, Pro, Ser, Thr, Trp, Tyr, or Val. In another aspect, the variant comprises or consists of the substitution L256M of the polypeptide of SEQ ID NO: 1.

In another aspect, the variant comprises or consists of a substitution at a position corresponding to position 358 of SEQ ID NO: 1. In another aspect, the amino acid at a position corresponding to position 358 is substituted with Ala, Arg, Asn, Asp, Cys, Gln, Glu, Gly, Ile, Leu, Lys, Met, Phe, Pro, Ser, Thr, Trp, Tyr, or Val. In another aspect, the variant comprises or consists of the substitution H358D or H358T of the polypeptide of SEQ ID NO: 1.

In another aspect, the variant comprises or consists of a substitution at a position corresponding to position 364 of SEQ ID NO: 1. In another aspect, the amino acid at a position corresponding to position 364 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A364G of the polypeptide of SEQ ID NO: 1.

In another aspect, the variant comprises or consists of a substitution at a position corresponding to position 366 of SEQ ID NO: 1. In another aspect, the amino acid at a position corresponding to position 366 is substituted with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of the substitution T3661 or T366V of the polypeptide of SEQ ID NO: 1.

In another aspect, the variant comprises or consists of a substitution at a position corresponding to position 375 of SEQ ID NO: 1. In another aspect, the amino acid at a position corresponding to position 375 is substituted with Ala, Arg, Asn, Asp, Gin, Glu, Gly, His, Ile, Leu, Lys, Met, Phe, Pro, Ser, Thr, Trp, Tyr, or Val. In another aspect, the variant comprises or consists of the substitution C375A or C375L or C375S or C375V of the polypeptide of SEQ ID NO: 1.

In another aspect, the variant comprises or consists of a substitution at a position corresponding to position 384 of SEQ ID NO: 1. In another aspect, the amino acid at a position corresponding to position 384 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q384A of the polypeptide of SEQ ID NO: 1.

In another aspect, the variant comprises or consists of a substitution at a position corresponding to position 385 of SEQ ID NO: 1. In another aspect, the amino acid at a position corresponding to position 385 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q385C of the polypeptide of SEQ ID NO: 1.

In another aspect, the variant comprises or consists of a substitution at a position corresponding to position 386 of SEQ ID NO: 1. In another aspect, the amino acid at a position corresponding to position 386 is substituted with Ala, Arg, Asn, Asp, Cys, Gln, Glu, Gly, His, Ile, Leu, Lys, Met, Phe, Pro, Ser, Thr, Trp, or Val. In another aspect, the variant comprises or consists of the substitution Y386G of the polypeptide of SEQ ID NO: 1.

In another aspect, the variant comprises or consists of a substitution at a position corresponding to position 388 of SEQ ID NO: 1. In another aspect, the amino acid at a position corresponding to position 388 is substituted with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of the substitution S388A or S388H or S388K or S388L or S388R of the polypeptide of SEQ ID NO: 1.

In another aspect, the variant comprises or consists of a substitution at a position corresponding to position 389 of SEQ ID NO: 1. In another aspect, the amino acid at a position corresponding to position 389 is substituted with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of the substitution S389H or S389V of the polypeptide of SEQ ID NO: 1.

In another aspect, the variant comprises or consists of a substitution at a position corresponding to position 391 of SEQ ID NO: 1. In another aspect, the amino acid at a position corresponding to position 391 is substituted with Ala, Arg, Asn, Asp, Cys, Gln, Glu, Gly, His, Ile, Leu, Lys, Met, Phe, Pro, Ser, Thr, Trp, or Val. In another aspect, the variant comprises or consists of the substitution Y391Vof the polypeptide of SEQ ID NO: 1.

In another aspect, the variant comprises or consists of a substitution at a position corresponding to position 393 of SEQ ID NO: 1. In another aspect, the amino acid at a position corresponding to position 393 is substituted with Ala, Arg, Asn, Asp, Cys, Gln, Glu, Gly, His, Ile, Leu, Lys, Met, Phe, Pro, Ser, Thr, Trp, or Tyr. In another aspect, the variant comprises or consists of the substitution V393H or V393R of the polypeptide of SEQ ID NO: 1.

In another aspect, the variant comprises or consists of a substitution at a position corresponding to position 394 of SEQ ID NO: 1. In another aspect, the amino acid at a position corresponding to position 394 is substituted with Ala, Arg, Asn, Asp, Cys, Gln, Glu, His, Ile, Leu, Lys, Met, Phe, Pro, Ser, Thr, Trp, Tyr, or Val. In another aspect, the variant comprises or consists of the substitution G394A or G394D or G394Q of the polypeptide of SEQ ID NO: 1.

In another aspect, the variant comprises or consists of a substitution at a position corresponding to position 395 of SEQ ID NO: 1. In another aspect, the amino acid at a position corresponding to position 395 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N395D of the polypeptide of SEQ ID NO: 1.

In another aspect, the variant comprises or consists of a substitution at a position corresponding to position 396 of SEQ ID NO: 1. In another aspect, the amino acid at a position corresponding to position 396 is substituted with Ala, Arg, Asn, Asp, Gln, Glu, Gly, His, Ile, Leu, Lys, Met, Phe, Pro, Ser, Thr, Trp, Tyr, or Val. In another aspect, the variant comprises or consists of the substitution C396A or C396L or C396S or C396V of the polypeptide of SEQ ID NO: 1.

In another aspect, the variant comprises or consists of a substitution at a position corresponding to position 446 of SEQ ID NO: 1. In another aspect, the amino acid at a position corresponding to position 446 is substituted with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of the substitution D446T of the polypeptide of SEQ ID NO: 1.

In another aspect, the variant comprises or consists of a substitution at a position corresponding to position 459 of SEQ ID NO: 1. In another aspect, the amino acid at a position corresponding to position 459 is substituted with Ala, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution R459V of the polypeptide of SEQ ID NO: 1.

In another aspect, the variant comprises or consists of a substitution at a position corresponding to position 468 of SEQ ID NO: 1. In another aspect, the amino acid at a position corresponding to position 468 is substituted with Ala, Arg, Asn, Asp, Gln, Glu, Gly, His, Ile, Leu, Lys, Met, Phe, Pro, Ser, Thr, Trp, Tyr, or Val. In another aspect, the variant comprises or consists of the substitution 0468A or C468L or C468S or C468V of the polypeptide of SEQ ID NO: 1.

In another aspect, the variant comprises or consists of a substitution at a position corresponding to position 469 of SEQ ID NO: 1. In another aspect, the amino acid at a position corresponding to position 469 is substituted with Ala, Arg, Asn, Asp, Cys, Gln, Glu, Gly, His, Leu, Lys, Met, Phe, Pro, Ser, Thr, Trp, Tyr, or Val. In another aspect, the variant comprises or consists of the substitution 1469T of the polypeptide of SEQ ID NO: 1.

In another aspect, the variant comprises or consists of a substitution at a position corresponding to position 470 of SEQ ID NO: 1. In another aspect, the amino acid at a position corresponding to position 470 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution D470A or D470K or D470T or D470V of the polypeptide of SEQ ID NO: 1.

In another aspect, the variant comprises or consists of a substitution at a position corresponding to position 471 of SEQ ID NO: 1. In another aspect, the amino acid at a position corresponding to position 471 is substituted with Ala, Arg, Asn, Asp, Cys, Gln, Glu, Gly, His, Ile, Lys, Met, Phe, Pro, Ser, Thr, Trp, Tyr, or Val. In another aspect, the variant comprises or consists of the substitution L471R of the polypeptide of SEQ ID NO: 1.

In another aspect, the variant comprises or consists of a substitution at a position corresponding to position 472 of SEQ ID NO: 1. In another aspect, the amino acid at a position corresponding to position 472 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A472I or A472L of the polypeptide of SEQ ID NO: 1.

In another aspect, the variant comprises or consists of a substitution at a position corresponding to position 473 of SEQ ID NO: 1. In another aspect, the amino acid at a position corresponding to position 473 is substituted with Ala, Arg, Asn, Asp, Cys, Gin, Glu, Gly, His, Ile, Leu, Lys, Met, Phe, Pro, Thr, Trp, Tyr, or Val. In another aspect, the variant comprises or consists of the substitution S473G or S473K of the polypeptide of SEQ ID NO: 1.

In another aspect, the variant comprises or consists of a substitution at a position corresponding to position 474 of SEQ ID NO: 1. In another aspect, the amino acid at a position corresponding to position 474 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N474G of the polypeptide of SEQ ID NO: 1.

In another aspect, the variant comprises or consists of a substitution at a position corresponding to position 476 of SEQ ID NO: 1. In another aspect, the amino acid at a position corresponding to position 476 is substituted with Ala, Arg, Asp, Cys, Gin, Glu, Gly, His, Ile, Leu, Lys, Met, Phe, Pro, Ser, Thr, Trp, Tyr, or Val. In another aspect, the variant comprises or consists of the substitution N476D of the polypeptide of SEQ ID NO: 1.

In another aspect, the variant comprises or consists of a substitution at a position corresponding to position 477 of SEQ ID NO: 1. In another aspect, the amino acid at a position corresponding to position 477 is substituted with Ala, Arg, Asn, Asp, Cys, Gin, Glu, Gly, His, Ile, Leu, Lys, Met, Phe, Pro, Ser, Trp, Tyr, or Val. In another aspect, the variant comprises or consists of the substitution T477K or T477V of the polypeptide of SEQ ID NO: 1.

In another aspect, the variant comprises or consists of a substitution at a position corresponding to position 478 of SEQ ID NO: 1. In another aspect, the amino acid at a position corresponding to position 478 is substituted with Ala, Arg, Asn, Asp, Cys, Gin, Glu, Gly, His, Ile, Lys, Met, Phe, Pro, Ser, Thr, Trp, Tyr, or Val. In another aspect, the variant comprises or consists of the substitution L478N or L478P of the polypeptide of SEQ ID NO: 1.

In another aspect, the variant comprises or consists of a substitution at a position corresponding to position 480 of SEQ ID NO: 1. In another aspect, the amino acid at a position corresponding to position 480 is substituted with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of the substitution T480L or T480N of the polypeptide of SEQ ID NO: 1.

In another aspect, the variant comprises or consists of a substitution at a position corresponding to position 481 of SEQ ID NO: 1. In another aspect, the amino acid at a position corresponding to position 481 is substituted with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of the substitution S481A of the polypeptide of SEQ ID NO: 1.

In another aspect, the variant comprises or consists of a substitution at a position corresponding to position 483 of SEQ ID NO: 1. In another aspect, the amino acid at a position corresponding to position 483 is substituted with Ala, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution R483V of the polypeptide of SEQ ID NO: 1.

In another aspect, the variant comprises or consists of a substitution at a position corresponding to position 484 of SEQ ID NO: 1. In another aspect, the amino acid at a position corresponding to position 484 is substituted with Ala, Arg, Asn, Asp, Cys, Gln, Glu, Gly, His, Ile, Lys, Met, Phe, Pro, Ser, Thr, Trp, Tyr, or Val. In another aspect, the variant comprises or consists of the substitution L484D or L484K or L484S of the polypeptide of SEQ ID NO: 1.

In another aspect, the variant comprises or consists of a substitution at a position corresponding to position 495 of SEQ ID NO: 1. In another aspect, the amino acid at a position corresponding to position 495 is substituted with Ala, Arg, Asn, Asp, Cys, Gln, Glu, Gly, His, Ile, Leu, Lys, Met, Phe, Pro, Ser, Thr, Tyr, or Val. In another aspect, the variant comprises or consists of the substitution W495E or W495I or W4955 of the polypeptide of SEQ ID NO: 1.

In another aspect, the variant comprises or consists of a substitution at a position corresponding to position 496 of SEQ ID NO: 1. In another aspect, the amino acid at a position corresponding to position 496 is substituted with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of the substitution Q496E or Q496W or Q496R of the polypeptide of SEQ ID NO: 1.

In another aspect, the variant comprises or consists of a substitution at a position corresponding to position 497 of SEQ ID NO: 1. In another aspect, the amino acid at a position corresponding to position 497 is substituted with Ala, Arg, Asn, Asp, Cys, Gln, Glu, Gly, His, Ile, Leu, Lys, Met, Phe, Pro, Ser, Thr, Trp, or Tyr. In another aspect, the variant comprises or consists of the substitution V497D or V497E or V497P or V497W of the polypeptide of SEQ ID NO: 1.

In another aspect, the variant comprises or consists of a substitution at a position corresponding to position 498 of SEQ ID NO: 1. In another aspect, the amino acid at a position corresponding to position 498 is substituted with Ala, Arg, Asn, Asp, Cys, Gln, Glu, Gly, His, Ile, Leu, Lys, Met, Phe, Pro, Ser, Thr, Trp, or Tyr. In another aspect, the variant comprises or consists of the substitution V498I of the polypeptide of SEQ ID NO: 1.

In another aspect, the variant comprises or consists of a substitution at a position corresponding to position 499 of SEQ ID NO: 1. In another aspect, the amino acid at a position corresponding to position 499 is substituted with Arg, Asn, Asp, Cys, Gin, Glu, Gly, His, Ile, Leu, Lys, Met, Phe, Pro, Ser, Thr, Trp, Tyr, or Val. In another aspect, the variant comprises or consists of the substitution A499L or A499Y of the polypeptide of SEQ ID NO: 1.

In another aspect, the variant comprises or consists of a substitution at a position corresponding to position 500 of SEQ ID NO: 1. In another aspect, the amino acid at a position corresponding to position 500 is substituted with Ala, Arg, Asn, Asp, Cys, Gin, Glu, Gly, His, Ile, Leu, Lys, Met, Phe, Pro, Ser, Thr, Trp, or Tyr. In another aspect, the variant comprises or consists of the substitution V500T of the polypeptide of SEQ ID NO: 1.

In another aspect, the variant comprises or consists of a substitution at a position corresponding to position 501 of SEQ ID NO: 1. In another aspect, the amino acid at a position corresponding to position 501 is substituted with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of the substitution 5501E or S501N of the polypeptide of SEQ ID NO: 1.

In another aspect, the variant comprises or consists of a substitution at a position corresponding to position 503 of SEQ ID NO: 1. In another aspect, the amino acid at a position corresponding to position 503 is substituted with Ala, Arg, Asn, Asp, Cys, Gln, Glu, His, Ile, Leu, Lys, Met, Phe, Pro, Ser, Thr, Trp, Tyr, or Val. In another aspect, the variant comprises or consists of the substitution G503L of the polypeptide of SEQ ID NO: 1.

In another aspect, the variant comprises or consists of a substitution at a position corresponding to position 522 of SEQ ID NO: 1. In another aspect, the amino acid at a position corresponding to position 522 is substituted with Ala, Arg, Asn, Asp, Gln, Glu, Gly, His, Ile, Leu, Lys, Met, Phe, Pro, Ser, Thr, Trp, Tyr, or Val. In another aspect, the variant comprises or consists of the substitution C522A or C522S or C522V of the polypeptide of SEQ ID NO: 1.

In another aspect, the variant comprises or consists of a substitution at a position corresponding to position 525 of SEQ ID NO: 1. In another aspect, the amino acid at a position corresponding to position 525 is substituted with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of the substitution D525L of the polypeptide of SEQ ID NO: 1.

In another aspect, the variant comprises or consists of a substitution at a position corresponding to position 526 of SEQ ID NO: 1. In another aspect, the amino acid at a position corresponding to position 526 is substituted with Ala, Arg, Asn, Asp, Cys, Gln, Glu, His, Ile, Leu, Lys, Met, Phe, Pro, Ser, Thr, Trp, Tyr, or Val. In another aspect, the variant comprises or consists of the substitution G526T of the polypeptide of SEQ ID NO: 1.

In another aspect, the variant comprises or consists of a substitution at a position corresponding to position 527 of SEQ ID NO: 1. In another aspect, the amino acid at a position corresponding to position 527 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N527H of the polypeptide of SEQ ID NO: 1.

In another aspect, the variant comprises or consists of a substitution at a position corresponding to position 528 of SEQ ID NO: 1. In another aspect, the amino acid at a position corresponding to position 528 is substituted with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of the substitution S528H of the polypeptide of SEQ ID NO: 1.

In another aspect, the variant comprises or consists of a substitution at a position corresponding to position 529 of SEQ ID NO: 1. In another aspect, the amino acid at a position corresponding to position 529 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N529T of the polypeptide of SEQ ID NO: 1.

In another aspect, the variant comprises or consists of a substitution at a position corresponding to position 530 of SEQ ID NO: 1. In another aspect, the amino acid at a position corresponding to position 530 is substituted with Ala, Arg, Asn, Asp, Cys, Gln, Glu, Gly, His, Ile, Lys, Met, Phe, Pro, Ser, Thr, Trp, Tyr, or Val. In another aspect, the variant comprises or consists of the substitution L530R of the polypeptide of SEQ ID NO: 1.

In another aspect, the variant comprises or consists of a substitution at a position corresponding to position 540 of SEQ ID NO: 1. In another aspect, the amino acid at a position corresponding to position 540 is substituted with Ala, Arg, Asn, Asp, Gin, Glu, Gly, His, Ile, Leu, Lys, Met, Phe, Pro, Ser, Thr, Trp, Tyr, or Val. In another aspect, the variant comprises or consists of the substitution C540A or C540L or C5405 or C540V of the polypeptide of SEQ ID NO: 1.

In one aspect, a variant comprises at least one substitution at a position corresponding to positions selected from a group consisting of: Y386G, Q385C, A276S, A276C, A276Q, A276F, A79C, A276N, V45L, 519T, H358D, L149R, A25W, V45N, I469T, S388A, A276T, S388H, S92D, D525L, S214Q, A364G, S388L, S388R, S473G, S389V, A20H, W495E, Y386Y, V393H, G187L, Q61K, G187S, A286E, A276Y, V258P, Q496E, G394A, V393R, T366V, S389S, S92N, N527H, Q384A, V497D, D470T, A18V, Q496Q, Q496R, S214E, P362P, S389H, V258R, A276D, L281V, V45I, and A117C of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+L149R, V45N+L149R, L149R+A364G, V258P+A276Q, V258P+A276S, A25W L+149R, V45L+A276S, A25W+A276S, V45L+A276Q, V45L+V258P, A25W+V258P, A25W+A276Q, L149R+W250Y, V45L+S388K, V45L+A364G, V45N+V258P, V258P+S388K, A25W+V45L, A276Q+S388K, W250Y+A276Q, A276S+S388K, A276Q+L281V, W250Y+A276S, A25W+S388K, A276S+L281V, G187S+A276Q, S92D+A276S, L149R+V258R, V45N+S388K, A25W+A364G, V258P+L281V, V258R+A276S, G187S+A276S, G187K+A276S, V45L+S92D, V45L+L281V, V45N+W250Y, V258R+A276Q, V45L+V258R, W250Y+S388K, V45N+G187K, A25W+V258R, G187K+S388K, A25W+S92D, L281V+S388K, V45N+V258R, L281V+A364G, S92D+A364G, A25W+G187S, A25W+G187K, W250Y+V258P, S92D+V258P, G187K+W250Y, V45N+S92D, G187S+A364G, G187S+L281V, G187S+S388K, G187S+V258R, S92D+G187S, V45L+D11E, L149R+S388K, L149R+D11E, A364G+D11E, and S388K+D11E of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+L149R+V258P, V45L+L149R+A364G, V45L+L149R+W250Y, V45L+L149R+G187S, V45L+L149R+S282N, and V45L+S92D+L149R of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitution at positions corresponding to positions selected from a group consisting of: V45L+L149R+A252S+H334D, V45L+L149R+S190E+D446T, V45L+L149R+V258P+A364G, V45L+L149R+A276Q+A364G, V45L+L149R+A276S+A364G, V45L+L149R+V258P+A276S, V45L+L149R L281V+A364G, L149R+V258P+A276S+A364G, V45L+V258P+A276S+A364G, A25W+V45L+L149R+A364G, V45L+L149R+V258R+A364G, V45L+L149R+G187K+A364G, V45L+L149R+G187S+A364G, and V45L+L149R+W250Y+A364G of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+A93S+L149R+A252S+H334D, V45L+A93S+L149R+A252S+H334D, V45L+A93S+L149R+V234P+A252S, V45L+A93S+L149R+A252S+H334D, V45L+A93S L149R+A252S+H334D, V45L+A93S+L149R+A252S+H334D, V45L+L149R5190E+D446T+R459V, V45L+L149R+V258P+A276S A364G, V45L+L149R+V258P+A276Q+A364G, A25W+V45L+L149R+V258P+A276S, V45L+L149R+A276Q+L281V+A364G, V45L+L149R+A276Q+S282N+A364G, A25W+V45L+L149R+V258P+A276Q, V45L+T70R+L149R+A276Q+A364G, V45L+L149R+V258P+L281V+A364G, A25W+V45L+L149R A276Q+A364G, V45L+L149R+A276Q+Q314A+A364G, V45L+L149R+G187K+V258P+A364G, V45L+L149R+A276Q+A286E+A364G, V45L+L149R+A276Q+H358T+A364G, A25W+V45N+L149R+V258P+A276S, V45L+N127D+L149R+A276Q+A364G, V45L+L149R+W250Y+A276Q+A364G, V45L+L149R+W250Y+V258P+A364G, V45L+L149R+A276Q+A364G+S388K, A25W+V45L+L149R+V258P+A364G, V45L+L149R+N168G+A276Q+A364G, V45L+A117S+L149R+A276Q+A364G, A24K+V45L+L149R+A276Q+A364G, V45L+L149R+A276Q+A286S+A364G, V45L+L149R+A170S+A276Q+A364G, V45L+L149R+T180Q+A276Q+A364G, V45L+L149R+A276Q V350L+A364G, V45L+L149R+V258P+S282N+A364G, A25W+V45L+L149R+A276S+A364G, V45L+L149R+G187S+V258P+A364G, A25W+L149R+V258P+A276S+A364G, V45L+L149R+G187L+A276Q A364G, A25W+V45L+V258P+A276S+A364G, V45L+L149R+N260H+A276Q+A364G, V45L+L149R+W250Y+V258R+A364G, V45L+L149R+N163H+A276Q+A364G, V45L+S92D+L149R+A276Q+A364G, V45L+L149R+S173Y+A276Q+A364G, V45L+S92D+L149R+V258P+A364G, V45L+D113T+L149R+A276Q+A364G, V45L+L149R+G187S+V258R+A364G, V45L+L149R+V258R+A364G+S388K, V45L+L149R+G187K V258R+A364G, V45L+L149R+V258R+S282N+A364G, V45L+L149R+V258R+L281V+A364G, V45L+S92N+L149R+V258P+A364G, V45L+L149R+V258P+A364G+S388K, and V45L+S92N+L149R+V258R+A364G of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: A25W+V45L+A79C+L149R+V258P+A276S, V45L+L149R+V258P+A276S+H358T+A364G, A25W+V45L+L149R+V258P+A276S+A364G, V45L+L149R+V258P+A276S+L281V+A364G, V45L+A93S+L149R+A252S+H334D+A340G, V45L+L149R+V258P+A276S+A364G+S388K, V45L+L149R+N168G+V258P+A276S+A364G, V45L+L149R+S173Y+V258P+A276S+A364G, V45L+A93S+L149R+A252S+H334D+A340G, A25W+V45L+L149R+V258P+A276S+H358T, A25W+V45L+L149R+V258P+A276Q+A364G, A25W+V45L+L149R+S214E+V258P+A276S, V45L+L149R+A170S+V258P+A276S+A364G, A25W+V45L+L149R+S214Q+V258P+A276S, A25W+V45L+L149R+V258P+A276S+S388K, V45L+A93S+L149R+V234P+A252S+H334D, V45L+N127D+L149R+V258P+A276S+A364G, V45L+L149R+W250Y+V258P+A276S+A364G, A25W+V45L+L149R+W250Y+A276Q+A364G, V45L+L149R+T180Q+V258P+A276S+A364G, V45L+L149R+V258P+A276S+Q314A+A364G, V45L+A117S+L149R+V258P+A276S+A364G, V45N+L149R+V258P+A276S+A364G+S388K, V45L+L149R+V258P+A276Q+L281V+A364G, A25W+V45L+L149R+V258P+A276S+Q314A, V45L+L149R+V258P+A276S+A286E+A364G, A25W+V45L+L149R+A276Q+L281V+A364G, A25W+V45L+L149R+V258P+A276S+A286E, A25W+V45L+L149R+V258P+A276S+L281V, A25W+V45L+D113T+L149R+V258P+A276S, A25W+V45L+L149R+T180Q+V258P+A276S, A25W+V45L+L149R+V258P+A276S+A286S, V45L+L149R+G187L+A276Q+L281V+A364G, V45L+L149R+A276Q+L281V+V350L+A364G, V45L+L149R+A276Q+L281V+A364G+S388K, A25W+V45L+S92D+L149R+V258P+A276S, A25W+V45L+L149R+G187L+V258P+A276S, A25W+V45L+L149R+W250Y+V258P+A276S, V45L+D113T+L149R+V258P+A276S+A364G, V45L+T70R+L149R+A276Q+L281V+A364G, V45L+S92D+L149R+A276Q+L281V+A364G, V45L+D113T+L149R+A276Q+L281V+A364G, V45L+L149R+A276Q+L281V+Q314A+A364G, A25W+V45L+Q61K+L149R+V258P+A276S, D9E+A25W+V45L+L149R+V258P+A276S, V45L+T70R+L149R+V258P+A276S+A364G, V45L+L149R+N163H+V258P+A276S+A364G, V45L+L149R+T180Q+A276Q+L281V+A364G, V45L+L149R+A276Q+L281V+A286S+A364G, A25W+V45L+L149R+A170S+V258P+A276S, A25W+V45L+T70R+L149R+V258P+A276S, V45L+L149R+N168G+A276Q+L281V+A364G, V45L+L149R+S173Y+A276Q+L281V+A364G, V45L+L149R+W250Y+A276Q+L281V+A364G, A25W+V45L+L149R+A276Q+A286E+A364G, A25W+V45L+L149R+A276Q+A364G+S388K, V45L+N127D+L149R+A276Q+L281V+A364G, V45L+L149R+A276Q+L281V+A286E+A364G, A25W+V45L+A117S+L149R+V258P+A276S, A24K+V45L+L149R+V258P+A276S+A364G, V45L+L149R+V258P+A276S+S282N+A364G, V45L+A117S+L149R+A276Q+L281V+A364G, V45L+L149R+N260H+A276Q+L281V+A364G, A25W+V45L+L149R+N168G+V258P+A276S, A25W+V45L+L149R+G187S+V258P+A276S, A25W+V45L+L149R+V258P+A276S+V350L, A25W+V45L+Q61R+L149R+V258P+A276S, V45N+L149R+V258P+A276S+L281V+A364G, A25W+V45L+L149R+V258P+A276S+S282N, A25W+V45L+L149R+S173Y+V258P+A276S, A25W+V45L+L149R+A276Q+A286S+A364G, A25W+V45L+L149R+T215V+V258P+A276S, A25W+V45L+L149R+T215I+V258P+A276S, A25W+V45L+L149R+T211H+V258P+A276S, V45L+L149R+V258P+A276S+V350L+A364G, A25W+V45L+D113T+L149R+A276Q+A364G, A25W+V45N+L149R+V258P+A276S+A364G, A25W+V45L+L149R+H219I+V258P+A276S, M28P+V45L+L149R+V258P+A276S+A364G, A25W+V45L+T70R+L149R+A276Q+A364G, A25W+V45L+L149R+A276Q+Q314A+A364G, A25W+V45L+L149R+S214Y+V258P+A276S, V45L+L149R+A276Q+L281V+H358T+A364G, A25W+V45L+L149R+H219V+V258P+A276S, D11E+A25W+V45L+L149R+V258P+A276S, A25W+V45L+A117S+L149R+A276Q+A364G, A25W+V45L+L149R+G187L+A276Q+A364G, V45N+L149R+V258P+A276S+H358T+A364G, A25W+V45L+L149R+N163H+A276Q+A364G, A25W+V45L+L149R+S173Y+A276Q+A364G, A25W+V45L+L149R+A276Q+S282N+A364G, A25W+V45L+L149R+A276Q+H358T+A364G, A25W+V45L+L149R+T180Q+A276Q+A364G, A25W+V45L+N127D+L149R+A276Q+A364G, A25W+V45L+L149R+A170S+A276Q+A364G, A25W+V45L+N127D+L149R+V258P+A276S, A25W+V45L+L149R+N168G+A276Q+A364G, A25W+V45L+L149R+N260H+A276Q+A364G, A25W+V45L+L149R+G187K+V258P+A276S, A25W+M28P+V45L+L149R+V258P+A276S, V45L+L149R+N163H+A276Q+L281V+A364G, V45L+L149R+G187L+V258P+A276S+A364G, A25W+V45L+L149R+A276Q+V350L+A364G, V45L+L149R+V258P+A276S+N355G+A364G, A25W+V45L+L149R+V258P+A276S+N355G, A25W+V45L+S92D+L149R+A276Q+A364G, and A25W+V45L+L149R+A276Q+N355G+A364G of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+A79C+L149R+V258P+A276S+A364G+S388K, V45N+A79C+L149R+V258P+A276S+A364G+S388K, V45L+L149R+S214Q+V258P+A276S+H358D+A364G, A25W+V45L+A79C+L149R+V258P+A276S+A364G, V45L+A79C+L149R+V258P+A276S+L281V+A364G, A25W+V45L+L149R+S214E+V258P+A276S+A364G, V45L+L149R+S214Q+V258P+A276Q+A364G+S388K, V45N+L149R+S214Q+V258P+A276S+A364G+S388K, V45L+L149R+S214Q+V258P+A276S+A364G+S388K, V45L+L149R+S214E+V258P+A276S+H358T+A364G, A25W+V45L+L149R+S214E+V258P+A276Q+A364G, V45L+L149R+S214Q+V258P+A276S+L281V+A364G, V45L+N127D+L149R+V258P+A276S+A364G+S388K, V45L+L149R+V258P+A276S+H358T+A364G+S388K, V45L+L149R+G187L+V258P+A276S+A364G+S388K, V45L+L149R+W250Y+V258P+A276S+A364G+S388K, V45L+L149R+V258P+A276S+A286E+A364G+S388K, V45L+L149R+V258P+A276S+S282N+A364G+S388K, A25W+V45L+L149R+V258P+A276S+A364G+S388K, V45L+L149R+S214Q+V258P+A276S+H358T+A364G, A25W+V45L+L149R+S214Q+V258P+A276S+A364G, V45L+L149R+N168G+V258P+A276S+A364G+S388K, V45L+A117S+L149R+V258P+A276S+A364G+S388K, A25W+V45L+L149R+V258P+A276S+H358T+A364G, V45L+L149R+V258P+N260H+A276S+A364G+S388K, V45L+L149R+T215I+V258P+A276S+A364G+S388K, V45L+Q61K+L149R+V258P+A276S+H358T+A364G, V45L+L149R+V258P+A276S+Q314A+H358T+A364G, V45L+L149R+T180Q+V258P+A276S+A364G+S388K, V45L+L149R+V258P+A276S+Q314A+A364G+S388K, A25W+V45L+L149R+V258P+A276S+A286S+A364G, V45L+L149R+H219I+V258P+A276S+H358T+A364G, V45L+L149R+V258P+A276S+V350L+A364G+S388K, D11E+V45L+L149R+V258P+A276S+A364G+S388K, V45L+D113T+L149R+V258P+A276S+A364G+S388K, V45L+L149R+G187K+V258P+A276S+A364G+S388K, M28P+V45L+L149R+V258P+A276S+A364G+S388K, V45L+L149R+S173Y+V258P+A276S+A364G+S388K, A25W+V45L+L149R+V258P+A276S+Q314A+A364G, V45L+L149R+G187L+V258P+A276S+H358T+A364G, V45L+L149R+T215V+V258P+A276S+H358T+A364G, V45L+T70R+L149R+V258P+A276S+A364G+S388K, V45L+L149R+V258P+A276S+V350L+H358T+A364G, D9E+V45L+L149R+V258P+A276S+H358T+A364G, V45L+L149R+T211H+V258P+A276S+H358T+A364G, V45L+L149R+V258P+A276S+L281V+A364G+S388K, V45L+L149R+V258P+A276S+S282N+H358T+A364G, V45L+L149R+T215I+V258P+A276S+H358T+A364G, A25W+V45L+L149R+S173Y+V258P+A276S+A364G, V45L+Q61K+L149R+V258P+A276S+A364G+S388K, A25W+V45L+Q61K+L149R+V258P+A276S+A364G, V45L+L149R+S214E+V258P+A276S+L281V+A364G, V45L+L149R+V258P+A276S+A286E+H358T+A364G, D9E+V45L+L149R+V258P+A276S+A364G+S388K, V45L+S92D+L149R+V258P+A276S+A364G+S388K, A25W+V45L+L149R+V258P+A276S+A286E+A364G, V45L+L149R+N168G+V258P+A276S+L281V+A364G, V45L+Q61R+L149R+V258P+A276S+H358T+A364G, V45L+L149R+G187S+V258P+A276S+A364G+S388K, V45L+L149R+V258P+A276S+L281V+Q314A+A364G, V45L+L149R+V258P+A276S+L281V+H358T+A364G, V45L+L149R+A170S+V258P+A276S+H358T+A364G, V45L+L149R+A170S+V258P+A276S+A364G+S388K, V45L+L149R+T215V+V258P+A276S+A364G+S388K, V45L+L149R+V258P+A276Q+H358T+A364G+S388K, A25W+V45N+L149R+S214E+V258P+A276S+A364G, V45L+L149R+V258P+A276S+A286S+H358T+A364G, A25W+V45L+L149R+G187S+V258P+A276S+A364G, V45L+L149R+T180Q+V258P+A276S+L281V+A364G, V45L+S92D+L149R+V258P+A276S+L281V+A364G, V45L+Q61R+L149R+V258P+A276S+L281V+A364G, V45L+L149R+A170S+V258P+A276S+L281V+A364G, D11E+A25W+V45L+L149R+V258P+A276S+A364G, A25W+V45L+Q61R+L149R+V258P+A276S+A364G, A25W+V45L+L149R+T215V+V258P+A276S+A364G, D9E+V45L+L149R+V258P+A276S+L281V+A364G, A25W+V45L+L149R+V258P+A276S+V350L+A364G, V45L+L149R+H219V+V258P+A276S+L281V+A364G, V45L+L149R+T180Q+V258P+A276S+H358T+A364G, V45L+L149R+T215V+V258P+A276S+L281V+A364G, A25W+V45L+L149R+V258P+A276S+S282N+A364G, A25W+V45L+L149R+T215I+V258P+A276S+A364G, A25W+V45L+L149R+H219I+V258P+A276S+A364G, V45L+A117S+L149R+V258P+A276S+L281V+A364G, D9E+A25W+V45L+L149R+V258P+A276S+A364G, V45L+L149R+S214Y+V258P+A276S+A364G+S388K, A25W+V45L+L149R+T180Q+V258P+A276S+A364G, A25W+V45L+L149R+V258P+A276S+L281V+A364G, V45L+N127D+L149R+V258P+A276S+L281V+A364G, V45L+L149R+S173Y+V258P+A276S+H358T+A364G, V45L+Q61R+L149R+V258P+A276S+A364G+S388K, V45L+L149R+V258P+A276S+L281V+A286E+A364G, A25W+V45L+L149R+W250Y+V258P+A276S+A364G, A25W+V45L+L149R+V258P+N260H+A276S+A364G, V45L+L149R+V258P+A276S+L281V+V350L+A364G, A25W+V45L+L149R+A170S+V258P+A276S+A364G, V45L+L149R+V258P+A276S+L281V+A286S+A364G, V45L+L149R+V258P+A276S+L281V+S282N+A364G, D11E+V45L+L149R+V258P+A276S+L281V+A364G, V45L+Q61K+L149R+V258P+A276S+L281V+A364G, V45L+L149R+T215I+V258P+A276S+L281V+A364G, A25W+V45L+S92D+L149R+V258P+A276S+A364G, V45L+L149R+S214Y+V258P+A276S+L281V+A364G, V45L+L149R+T211H+V258P+A276S+L281V+A364G, A25W+V45L+L149R+G187L+V258P+A276S+A364G, V45L+S92D+L149R+V258P+A276S+H358T+A364G, V45L+L149R+G187K+V258P+A276S+H358T+A364G, V45L+L149R+N163H+V258P+A276S+L281V+A364G, A25W+V45L+T70R+L149R+V258P+A276S+A364G, V45L+L149R+S173Y+V258P+A276S+L281V+A364G, V45L+L149R+G187L+V258P+A276S+L281V+A364G, V45L+L149R+G187K+V258P+A276S+L281V+A364G, V45L+L149R+W250Y+V258P+A276S+H358T+A364G, V45L+L149R+T211H+V258P+A276S+A364G+S388K, V45L+L149R+N163H+V258P+A276S+A364G+S388K, V45L+L149R+V258P+A276S+A286S+A364G+S388K, A25W+V45L+L149R+S214Y+V258P+A276S+A364G, A25W+V45L+D113T+L149R+V258P+A276S+A364G, V45L+T70R+L149R+V258P+A276S+L281V+A364G, A25W+V45L+N127D+L149R+V258P+A276S+A364G, V45L+T70R+L149R+V258P+A276S+H358T+A364G, V45L+L149R+H219I+V258P+A276S+L281V+A364G, A25W+V45L+A117S+L149R+V258P+A276S+A364G, A25W+V45L+L149R+N168G+V258P+A276S+A364G, V45L+N127D+L149R+V258P+A276S+H358T+A364G, A25W+V45L+L149R+H219V+V258P+A276S+A364G, D11E+V45L+L149R+V258P+A276S+H358T+A364G, V45L+L149R+G187S+V258P+A276S+L281V+A364G, A25W+V45L+L149R+N163H+V258P+A276S+A364G, A25W+V45L+L149R+G187K+V258P+A276S+A364G, V45L+L149R+V258P+N260H+A276S+H358T+A364G, V45L+D113T+L149R+V258P+A276S+L281V+A364G, V45L+D113T+L149R+V258P+A276S+H358T+A364G, V45L+L149R+N168G+V258P+A276S+H358T+A364G, V45L+L149R+S214Y+V258P+A276S+H358T+A364G, V45L+A117S+L149R+V258P+A276S+H358T+A364G, A25W+M28P+V45L+L149R+V258P+A276S+A364G, M28P+V45L+L149R+V258P+A276S+L281V+A364G, M28P+V45L+L149R+V258P+A276S+H358T+A364G, V45L+L149R+N163H+V258P+A276S+H358T+A364G, V45L+L149R+T215K+V258P+A276S+H358T+A364G, V45L+L149R+H219I+V258P+A276S+A364G+S388K, V45L+L149R+T215K+V258P+A276S+A364G+S388K, V45L+L149R+W250Y+V258P+A276S+L281V+A364G, V45L+L149R+H219V+V258P+A276S+A364G+S388K, A25W+V45L+L149R+V258P+A276S+N355G+A364G, V45L+L149R+V258P+A276S+N355G+A364G+S388K, V45L+L149R+V258P+A276S+L281V+N355G+A364G, V45L+L149R+V258P+A276S+N355G+H358T+A364G, and V45L+L149R+H219V+V258P+A276S+H358T+A364G of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+A79C+L149R+S214E+V258P+A276S+A364G+S388K, V45L+A79C+L149R+S214Q+V258P+A276S+A364G+S388K, V45L+A79C+L149R+V258P+A276S+Q314A+A364G+S388K, V45L+A79C+L149R+V258P+A276S+L281V+A364G+S388K, V45L+A79C+L149R+S173Y+V258P+A276S+A364G+S388K, V45L+A79C+L149R+G187K+V258P+A276S+A364G+S388K, V45L+Q61R+A79C+L149R+V258P+A276S+A364G+S388K, V45L+A79C+L149R+N168G+V258P+A276S+A364G+S388K, V45L+A79C+L149R+T215V+V258P+A276S+A364G+S388K, V45L+A79C+L149R+V258P+A276S+A286S+A364G+S388K, V45L+A79C+L149R+H219V+V258P+A276S+A364G+S388K, V45L+A79C+L149R+A170S+V258P+A276S+A364G+S388K, D11E+V45L+A79C+L149R+V258P+A276S+A364G+S388K, V45L+A79C+L149R+V258P+A276S+A286E+A364G+S388K, V45L+A79C+L149R+T180Q+V258P+A276S+A364G+S388K, V45L+A79C+L149R+H219I+V258P+A276S+A364G+S388K, V45L+L149R+S214E+V258P+A276S+H358D+A364G+D470T, V45L+A79C+L149R+V258P+A276S+V350L+A364G+S388K, V45L+L149R+S214E+V258P+A276S+H358D+A364G+D470T, V45L+A79C+D113T+L149R+V258P+A276S+A364G+S388K, V45L+A79C+N127D+L149R+V258P+A276S+A364G+S388K, D9E+V45L+A79C+L149R+V258P+A276S+A364G+S388K, V45L+L149R+S214E+V258P+A276S+H358D+A364G+D470T, V45L+A79C+L149R+W250Y+V258P+A276S+A364G+S388K, V45L+L149R+S214E+V258P+A276S+H358D+A364G+D470T, V45L+L149R+S214E+V258P+A276S+H358D+A364G+D470T, V45L+A79C+L149R+N163H+V258P+A276S+A364G+S388K, V45L+A117S+L149R+S214Q+V258P+A276S+H358D+A364G, A25W+V45L+A79C+L149R+S214E+V258P+A276S+A364G, V45L+A117S+L149R+S214Q+V258P+A276S+A364G+D470T, V45L+A79C+S92D+L149R+V258P+A276S+A364G+S388K, V45L+A117S+L149R+S214Q+V258P+A276S+H358D+A364G, V45L+L149R+S214Q+V258P+A276S+A286E+A364G+S388K, V45L+L149R+S214Q+T215I+V258P+A276S+A364G+S388K, A25W+V45L+L149R+S214Q+V258P+A276S+A364G+S388K, V45L+L149R+A170S+S214Q+V258P+A276S+A364G+S388K, V45L+Q61K+L149R+S214Q+V258P+A276S+A364G+S388K, V45L+L149R+N168G+S214Q+V258P+A276S+A364G+S388K, V45L+A79C+D95D+L149R+V258P+A276S+A364G+S388K, V45L+Q77E+A79C+L149R+V258P+A276S+A364G+S388K, V45L+L149R+S214Q+V258P+A276S+S282N+A364G+S388K, V45L+L149R+S214Q+V258P+A276S+Q314A+A364G+S388K, V45L+T70R+L149R+S214Q+V258P+A276S+A364G+S388K, V45L+L149R+S214Q+W250Y+V258P+A276S+A364G+S388K, V45L+L149R+G187K+S214Q+V258P+A276S+A364G+S388K, V45L+L149R+V258P+A276S+L281V+H358T+A364G+S388K, M28P+V45L+L149R+S214Q+V258P+A276S+A364G+S388K, V45L+L149R+G187L+S214Q+V258P+A276S+A364G+S388K, V45L+L149R+S214Q+V258P+A276S+A286S+A364G+S388K, D9E+V45L+L149R+S214Q+V258P+A276S+A364G+S388K, V45L+L149R+S173Y+S214Q+V258P+A276S+A364G+S388K, V45L+A79C+L149R+V258P+A276S+H358T+A364G+S388K, A25W+V45L+L149R+S214E+V258P+A276S+H358T+A364G, A25W+V45L+L149R+S214E+V258P+A276S+A286E+A364G, A25W+V45L+Q61K+L149R+S214E+V258P+A276S+A364G, A25W+V45L+L149R+V258P+A276S+H358T+A364G+S388K, V45L+L149R+S214Q+V258P+A276S+V350L+A364G+S388K, D11E+V45L+L149R+S214Q+V258P+A276S+A364G+S388K, V45L+L149R+H219I+V258P+A276S+H358T+A364G+S388K, V45L+S92D+L149R+S214Q+V258P+A276S+A364G+S388K, A25W+V45L+L149R+T211H+S214E+V258P+A276S+A364G, A25W+V45L+L149R+S214E+H219V+V258P+A276S+A364G, V45L+L149R+W250Y+V258P+A276S+H358T+A364G+S388K, D9E+V45L+L149R+V258P+A276S+H358T+A364G+S388K, V45L+L149R+H219V+V258P+A276S+H358T+A364G+S388K, V45L+A117S+L149R+S214E+V258P+A276S+A364G+S388K, D11E+A25W+V45L+L149R+S214E+V258P+A276S+A364G, V45L+L149R+V258P+A276S+Q314A+H358T+A364G+S388K, V45L+A117S+L149R+S214Q+V258P+A276S+A364G+S388L, V45L+L149R+T180Q+S214Q+V258P+A276S+A364G+S388K, V45L+D113T+L149R+S214Q+V258P+A276S+A364G+S388K, A25W+V45L+Q61R+L149R+S214E+V258P+A276S+A364G, A25W+V45L+L149R+S214E+V258P+A276S+L281V+A364G, D9E+A25W+V45L+L149R+S214E+V258P+A276S+A364G, V45L+Q61R+L149R+V258P+A276S+H358T+A364G+S388K, V45L+D113T+L149R+V258P+A276S+H358T+A364G+S388K, V45L+A117S+L149R+S214Q+V258P+A276S+A364G+S388R, V45L+Q61R+L149R+S214Q+V258P+A276S+A364G+S388K, V45L+A117S+L149R+S214Q+V258P+A276Q+A364G+S388K, V45L+A117S+L149R+S214Q+V258P+A276S+A364G+S388K, V45L+L149R+T180Q+V258P+A276S+H358T+A364G+S388K, A25W+V45L+S92D+L149R+S214E+V258P+A276S+A364G, A25W+V45L+L149R+S214E+V258P+A276S+V350L+A364G, V45L+L149R+T211H+S214Q+V258P+A276S+A364G+S388K, A25W+V45L+L149R+S214E+H219I+V258P+A276S+A364G, V45L+L149R+T211H+V258P+A276S+H358T+A364G+S388K, A25W+V45L+A117S+L149R+S214E+V258P+A276S+A364G, A25W+V45L+L149R+S214E+T215V+V258P+A276S+A364G, V45L+A117S+L149R+V258P+A276S+H358T+A364G+S388K, V45L+L149R+N168G+V258P+A276S+H358T+A364G+S388K, V45L+A117S+L149R+S214Q+V258P+A276S+A364G+S388H, A25W+V45L+L149R+S214E+T215I+V258P+A276S+A364G, V45L+N127D+L149R+V258P+A276S+H358T+A364G+S388K, V45L+A117S+L149R+S214Q+V258P+A276S+A364G+S388A, V45N+A117S+L149R+S214Q+V258P+A276S+A364G+S388K, A25W+V45L+L149R+G187S+S214E+V258P+A276S+A364G, V45L+A117S+L149R+S214Y+V258P+A276S+A364G+S388K, A25W+V45L+L149R+S214E+V258P+A276S+A286S+A364G, V45L+L149R+V258P+A276S+A286S+H358T+A364G+S388K, V45L+L149R+S173Y+V258P+A276S+H358T+A364G+S388K, V45L+A79C+L149R+V258P+A276S+N355G+A364G+S388K, A25W+V45L+L149R+G187L+S214E+V258P+A276S+A364G, V45L+L149R+S214Q+V258P+A276S+L281V+A364G+S388K, V45L+L149R+S214Q+T215V+V258P+A276S+A364G+S388K, V45L+L149R+N163H+V258P+A276S+H358T+A364G+S388K, V45L+A79C+A117S+L149R+V258P+A276S+A364G+S388K, V45L+L149R+A170S+V258P+A276S+H358T+A364G+S388K, A25W+V45L+L149R+S214E+T215K+V258P+A276S+A364G, V45L+S92D+L149R+V258P+A276S+H358T+A364G+S388K, V45L+L149R+S214Q+V258P+A276S+N355G+A364G+S388K, A25W+V45L+L149R+S214E+V258P+A276S+N355G+A364G, and A25W+V45L+L149R+S214E+V258P+A276S+Q314A+A364G of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+A117S+L149R+S214Q+V258P+A276S+A364G+S388R+D470T, V45L+A117S+L149R+S214Q+V258P+A276S+A364G+S388K+D470T, V45L+A117S+L149R+S214Q+V258P+A276S+A364G+S388L+D470T, V45L+A117S+L149R+S214Q+V258P+A276Q+A364G+S388K+D470T, V45L+A117S+L149R+D192E+S214Q+V258P+A276S+H358D+A364G, V45L+A117S+L149R+S214Q+V258P+A276S+A364G+S388A+D470T, V45L+A117S+L149R+S214Q+V258P+A276S+A364G+S388H+D470T, V45N+A117S+L149R+S214Q+V258P+A276S+A364G+S388K+D470T, V45L+A117S+L149R+S214E+V258P+A276S+H358D+A364G+S388K, V45L+A117S+L149R+D192E+S214Q+V258P+A276S+H358D+A364G, V45L+A117S+L149R+S214Q+V258P+A276S+H358D+A364G+S388K, V45L+A117S+L149R+S214Q+V258P+A276S+A364G+S388K+R483V, V45L+A117S+L149R+S214E+V258P+A276S+A364G+S388K+D470T, V45L+A117S+L149R+S214Q+V258P+A276S+H358D+A364G+S388R, V45L+A117S+L149R+S214Q+V258P+A276S+H358D+A364G+S388A, V45L+A117S+L149R+S214Q+V258P+A276S+A364G+Q384A+S388K, V45L+A117S+L149R+S214Q+V258P+A276S+H358D+A364G+S388L, V45L+A117S+L149R+S214Q+V258P+A276S+A286E+A364G+S388K, V45L+A117S+L149R+S214Q+V258P+A276S+L281V+A364G+S388K, V45L+A117S+L149R+S214Q+V258P+A276S+A364G+S388K+G394D, V45L+A117S+L149R+S214Q+V258P+A276S+A364G+S388K+S473G, V45L+A117S+L149R+S214Q+V258P+A276S+A364G+S388K+G394A, V45N+A117S+L149R+S214Q+V258P+A276S+H358D+A364G+S388K, V45L+A117S+L149R+S214Q+V258P+A276S+A364G+S388K+L478P, V45L+A117S+L149R+S214Q+V258P+A276S+A364G+S388K+S481A, V45L+A117S+L149R+S214Q+V258P+A276S+Q314A+A364G+S388K, V45L+A117S+L149R+S214Q+V258P+A276Q+H358D+A364G+S388K, V45L+A117S+L149R+G187K+S214Q+V258P+A276S+A364G+S388K, V45L+A117S+L149R+S214Q+V258P+A276S+A364G+S388K+L478N, V45L+A117S+L149R+S214Q+V258P+A276S+A364G+S388K+V393R, V45L+A117S+L149R+S214Q+V258P+A276S+A364G+T366V+S388K, V45L+A117S+L149R+S214Q+V258P+A276S+H358T+A364G+S388K, D11E+V45L+A117S+L149R+S214Q+V258P+A276S+A364G+S388K, V45L+A117S+L149R+S214Q+V258P+A276S+A364G+S388K+G394Q, V45L+A117S+L149R+S214Q+V258P+A276S+A364G+S388K+N395D, V45L+A117S+L149R+S214Q+V258P+A276S+A364G+S388K+V393H, V45L+A117S+L149R+S214Q+V258P+A276S+A364G+S388K+S501E, V45L+A117S+L149R+S214Q+V258P+A276S+A364G+S388K+S528H, V45L+A117S+L149R+S214Q+V258P+A276S+H358D+A364G+S388H, V45L+A117S+L149R+S214Q+V258P+A276S+A364G+S388K+S389H, V45L+A117S+L149R+S214Q+V258P+A276S+A364G+T366I+S388K, V45L+A117S+L149R+S214Q+V258P+A276S+A364G+S388K+N474G, V45L+A117S+L149R+S214Y+V258P+A276S+H358D+A364G+S388K, V45L+A117S+L149R+S214Q+V258P+A276S+A286S+A364G+S388K, V45L+A117S+L149R+S214Q+V258P+A276S+A364G+S388K+A499Y, V45L+A117S+L149R+S214Q+V258P+A276S+A364G+S388K+V500V, V45L+A117S+L149R+S214Q+W250Y+V258P+A276S+A364G+S388K, V45L+A117S+L149R+S214Q+V258P+A276S+A364G+S388K+T480N, V45L+A117S+L149R+G187S+S214Q+V258P+A276S+A364G+S388K, A25W+V45L+A117S+L149R+S214Q+V258P+A276S+A364G+S388K, V45L+A117S+L149R+S214Q+V258P+A276S+A364G+S388K+N476N, V45L+A117S+L149R+S214Q+V258P+A276S+A364G+S388K+N476D, D9E+V45L+A117S+L149R+S214Q+V258P+A276S+A364G+S388K, V45L+A117S+L149R+S214Q+V258P+A276S+A364G+S388K+S481S, V45L+A117S+L149R+S214Q+H219V+V258P+A276S+A364G+S388K, V45L+A117S+L149R+S214Q+T215I+V258P+A276S+A364G+S388K, V45L+A117S+L149R+S214Q+V258P+A276S+A364G+S388K+Q496R, V45L+A117S+L149R+A170S+S214Q+V258P+A276S+A364G+S388K, V45L+Q61R+A117S+L149R+S214Q+V258P+A276S+A364G+S388K, V45L+A117S+L149R+S214Q+V258P+A276S+A364G+S388K+V497P, V45L+A117S+L149R+T180Q+S214Q+V258P+A276S+A364G+S388K, V45L+A117S+L149R+S214Q+T215V+V258P+A276S+A364G+S388K, V45L+A117S+L149R+S214Q+V258P+A276S+V350L+A364G+S388K, V45L+Q61K+A117S+L149R+S214Q+V258P+A276S+A364G+S388K, V45L+A117S+L149R+S214Q+V258P+A276S+A364G+S388K+Y391V, V45L+A117S+L149R+N168G+S214Q+V258P+A276S+A364G+S388K, V45L+D113T+A117S+L149R+S214Q+V258P+A276S+A364G+S388K, M28P+V45L+A117S+L149R+S214Q+V258P+A276S+A364G+S388K, V45L+A117S+L149R+S214Q+V258P+A276S+A364G+S388K+T477K, V45L+A117S+L149R+S214Q+V258P+A276S+A364G+S388K+S389V, V45L+A117S+L149R+S173Y+S214Q+V258P+A276S+A364G+S388K, V45L+A117S+L149R+S214Q+V258P+A276S+A364G+S388K+V500T, V45L+A117S+L149R+T211H+S214Q+V258P+A276S+A364G+S388K, V45L+S92D+A117S+L149R+S214Q+V258P+A276S+A364G+S388K, V45L+A117S+L149R+S214Q+V258P+A276S+A364G+S388K+A472L, V45L+A117S+L149R+S214Q+V258P+A276S+A364G+S388K+S473K, V45L+A117S+L149R+S214Q+V258P+A276S+A364G+S388K+A499L, V45L+A117S+L149R+S214Q+V258P+A276S+A364G+S388K+V498I, V45L+A117S+L149R+N163H+S214Q+V258P+A276S+A364G+S388K, V45L+A117S+L149R+S214Q+V258P+A276S+A364G+S388K+N527H,V45L+A117S+L149R+S214Q+V258P+A276S+A364G+S388K+T477V, V45L+A117S+L149R+S214Q+V258P+A276S+A364G+S388K+G503L, V45L+A117S+L149R+G187L+S214Q+V258P+A276S+A364G+S388K, V45L+A117S+L149R+S214Q+T215K+V258P+A276S+A364G+S388K, V45L+A117S+L149R+S214Q+V258P+A276S+A364G+Q385C+S388K, and V45L+A117S+L149R+S214Q+V258P+A276S+N355G+A364G+S388K of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+A117S+L149R+S214E+V258P+A276S+H358D+A364G+S388K+D470T, V45L+A117S+L149R+S214Q+V258P+A276S+H358D+A364G+S388R+D470T, V45L+A117S+L149R+S214Q+V258P+A276Q+H358D+A364G+S388K+D470T, V45L+A117S+L149R+S214Q+V258P+A276S+H358D+A364G+S388H+D470T, V45L+A79C+A117S+L149R+S214Q+V258P+A276S+H358D+A364G+S388K, V45L+A117S+L149R+S214Q+V258P+A276S+H358D+A364G+S388L+D470T, V45L+A117S+L149R+S214E+V258P+A276S+A364G+S388K+D470T+R483V, V45L+A117S+L149R+S214Q+V258P+A276S+A364G+S388R+D470T+R483V, V45L+A117S+L149R+S214Q+V258P+A276S+A364G+S388K+D470A+R483V, V45L+A117S+L149R+S214Q+V258P+A276Q+A364G+S388K+D470T+R483V, V45L+A117S+L149R+S214Q+V258P+A276S+A364G+S388K+D470T+R483V, V45L+A117S+L149R+S214Q+V258P+A276S+H358D+A364G+S388K+D470T, V45N+A117S+L149R+S214Q+V258P+A276S+A364G+S388K+D470T+R483V, V45L+A117S+L149R+S214Q+V258P+A276S+A364G+S388H+D470T+R483V, V45L+A79C+A117S+L149R+S214Q+V258P+A276S+A364G+S388K+D470T, V45L+A117S+L149R+S214Q+V258P+A276S+H358D+A364G+S388A+D470T, V45L+A117S+L149R+S214Q+V258P+A276S+A364G+S388A+D470T+R483V, V45L+A117S+L149R+S214Q+V258P+A276S+L281V+A364G+S388K+D470T, V45L+A117S+L149R+S214Q+V258P+A276S+A364G+Q384A+S388K+D470T, V45L+A117S+L149R+S214Q+V258P+A276S+A286E+A364G+S388K+D470T, V45L+A117S+L149R+S214Q+V258P+A276S+A364G+S388K+D470T+T477K, V45L+A117S+L149R+S214Y+V258P+A276S+H358D+A364G+S388K+D470T, V45L+A117S+L149R+S214Q+V258P+A276S+A364G+S388K+G394A+D470T, V45L+A117S+L149R+S214Q+V258P+A276S+A364G+S388K+D470T+N476D, V45L+A117S+L149R+S214Q+V258P+A276S+A364G+S388K+V393H+D470T, V45L+A117S+L149R+S214Q+V258P+A276S+A364G+S388K+N395D+D470T, V45L+A117S+L149R+S214Q+V258P+A276S+H358T+A364G+S388K+D470T, V45L+A117S+L149R+S214Q+V258P+A276S+A364G+S388K+D470T+L478N, V45L+A117S+L149R+S214Q+W250Y+V258P+A276S+A364G+S388K+D470T, V45L+A117S+L149R+G187L+S214Q+V258P+A276S+A364G+S388K+D470T, D9E+V45L+A117S+L149R+S214Q+V258P+A276S+A364G+S388K+D470T, V45L+A117S+L149R+S214Q+V258P+A276S+S282N+A364G+S388K+D470T, D11E+V45L+A117S+L149R+S214Q+V258P+A276S+A364G+S388K+D470T, V45L+A117S+L149R+S214Q+V258P+A276S+A364G+S388K+D470T+N476N, V45L+A117S+L149R+S214Q+V258P+A276S+A364G+S388K+G394D+D470T, V45L+Q61K+A117S+L149R+S214Q+V258P+A276S+A364G+S388K+D470T, V45L+A117S+L149R+S214Q+V258P+A276S+A286S+A364G+S388K+D470T, V45L+A117S+L149R+S214Q+V258P+A276S+H358D+A364G+S388K+D470A, V45L+A117S+L149R+S214Q+V258P+A276S+A364G+S388K+G394Q+D470T, V45L+A117S+L149R+S214Q+V258P+A276S+A364G+S388K+D470T+L478P, V45L+A117S+L149R+S214Q+V258P+A276S+V350L+A364G+S388K+D470T, V45L+D113T+A117S+L149R+S214Q+V258P+A276S+A364G+S388K+D470T, V45L+A117S+L149R+T180Q+S214Q+V258P+A276S+A364G+S388K+D470T, V45L+A117S+L149R+S214Q+V258P+A276S+A364G+S388K+S389H+D470T, V45L+A117S+L149R+S214Q+V258P+A276S+A364G+S388K+V393R+D470T, V45L+A117S+L149R+S214Q+V258P+A276S+A364G+S388K+D470T+Q496R, V45L+A117S+N127D+L149R+S214Q+V258P+A276S+A364G+S388K+D470T, V45L+A117S+L149R+S214Q+V258P+A276S+A364G+S388K+D470T+A499Y, V45L+A117S+L149R+S173Y+S214Q+V258P+A276S+A364G+S388K+D470T, V45L+S92D+A117S+L149R+S214Q+V258P+A276S+A364G+S388K+D470T, V45L+A117S+L149R+S214Q+V258P+A276S+A364G+S388K+D470T+N529T, V45L+A117S+L149R+S214Q+V258P+A276S+H358D+A364G+S388K+L478N, V45L+T70R+A117S+L149R+S214Q+V258P+A276S+A364G+S388K+D470T, M28P+V45L+A117S+L149R+S214Q+V258P+A276S+A364G+S388K+D470T, V45L+A117S+L149R+S214Y+V258P+A276S+A364G+S388K+D470T+R483V, V45L+A117S+L149R+S214Q+V258P+A276S+H358D+A364G+S388K+N395D, V45L+A117S+L149R+S214Q+V258P+A276S+H358D+A364G+S388K+T477K, V45L+A117S+L149R+S214Q+V258P+A276S+L281V+H358D+A364G+S388K, V45L+Q61R+A117S+L149R+S214Q+V258P+A276S+A364G+S388K+D470T, V45L+A117S+L149R+S214Q+T215I+V258P+A276S+A364G+S388K+D470T, V45L+A117S+L149R+S214Q+V258P+A276S+A364G+S388K+D470T+Q496E, V45L+A117S+L149R+G187K+S214Q+V258P+A276S+A364G+S388K+D470T, V45L+A117S+L149R+S214Q+V258P+A276S+A364G+S388K+D470T+W495I, V45L+A117S+L149R+S214Q+V258P+A276S+A364G+S388K+D470T+V498I, V45L+A117S+L149R+S214Q+V258P+A276S+A364G+S388K+D470T+V500V, D11E+V45L+A117S+L149R+S214Q+V258P+A276S+H358D+A364G+S388K, V45L+A117S+L149R+A170S+S214Q+V258P+A276S+A364G+S388K+D470T, V45L+A117S+L149R+S214Q+V258P+A276S+A364G+S388K+Y391V+D470T, V45L+A117S+L149R+S214Q+V258P+A276S+Q314A+A364G+S388K+D470T, V45L+A117S+L149R+S214Q+V258P+A276S+A364G+S388K+D470T+T477V, V45L+A117S+L149R+S214Q+V258P+A276S+H358D+A364G+S388K+G394Q, V45L+A117S+L149R+S214Q+H219V+V258P+A276S+A364G+S388K+D470T, V45L+A117S+L149R+S214Q+V258P+A276S+A364G+S388K+D470T+Q496Q, V45L+A117S+L149R+S214Q+V258P+A276S+H358D+A364G+S388K+A499Y, V45L+A117S+L149R+S214Q+T215V+V258P+A276S+A364G+S388K+D470T, V45L+A117S+L149R+S214Q+V258P+A276S+A364G+Q385C+S388K+D470T, V45L+A117S+L149R+S214Q+V258P+A276S+A364G+S388K+D470T+T480N, V45L+A117S+L149R+S214Q+V258P+A276S+A364G+S388K+D470T+V497P, V45L+A117S+L149R+S214Q+V258P+A276S+A364G+S388K+D470T+A499L, V45L+A117S+L149R+S214Q+V258P+A276S+A364G+S388K+D470T+S501N, V45L+A117S+L149R+S214Q+V258P+A276S+H358D+A364G+Q384A+S388K, V45L+A117S+L149R+S214Q+V258P+A276S+A364G+S388L+D470T+R483V, V45L+A117S+L149R+S214Q+V258P+A276S+H358D+A364G+S388K+G394A, V45L+A117S+L149R+S214Q+V258P+A276S+H358D+A364G+S388K+V498I, V45L+A117S+L149R+S214Q+H219I+V258P+A276S+A364G+S388K+D470T, V45L+A117S+L149R+N163H+S214Q+V258P+A276S+A364G+S388K+D470T, V45L+A117S+L149R+S214Q+V258P+A276S+Q314A+H358D+A364G+S388K, V45L+A117S+L149R+S173Y+S214Q+V258P+A276S+H358D+A364G+S388K, V45L+A117S+L149R+S214Q+V258P+A276S+H358D+A364G+S388K+G394D, V45L+Q61R+A117S+L149R+S214Q+V258P+A276S+H358D+A364G+S388K, V45L+A117S+L149R+S214Q+V258P+A276S+H358D+A364G+T366V+S388K, V45L+A117S+L149R+T180Q+S214Q+V258P+A276S+H358D+A364G+S388K, V45L+A117S+L149R+S214Q+T215I+V258P+A276S+H358D+A364G+S388K, V45L+A117S+L149R+S214Q+W250Y+V258P+A276S+H358D+A364G+S388K, V45L+A117S+L149R+S214Q+V258P+A276S+H358D+A364G+S388K+N476D, V45L+A117S+L149R+S214Q+V258P+A276S+H358D+A364G+S388K+T477V, V45L+A117S+L149R+S214Q+V258P+A276S+H358D+A364G+S388K+L478P, V45L+A117S+L149R+S214Q+V258P+A276S+H358D+A364G+S388K+S481A, V45L+A117S+L149R+T211H+S214Q+V258P+A276S+A364G+S388K+D470T, V45L+A117S+L149R+S214Q+V258P+A276S+H358D+A364G+S388K+V500T, V45L+A117S+L149R+S214Q+V258P+A276S+V350L+H358D+A364G+S388K, V45L+A117S+L149R+G187K+S214Q+V258P+A276S+H358D+A364G+S388K, V45L+A117S+L149R+S214Q+V258P+A276S+A364G+S388K+D470V+R483V, V45L+A117S+L149R+S214Q+V258P+A276S+H358D+A364G+S388K+V500V, V45L+A117S+L149R+N163H+S214Q+V258P+A276S+H358D+A364G+S388K, V45L+A117S+L149R+S214Q+V258P+A276S+H358D+A364G+S388K+S481S, V45L+A117S+L149R+S214Q+V258P+A276S+A364G+S388K+D470T+V500T, V45L+A117S+L149R+S214Q+V258P+A276S+H358D+A364G+S388K+W495S, V45L+D113T+A117S+L149R+S214Q+V258P+A276S+H358D+A364G+S388K, V45L+A117S+L149R+S214Q+V258P+A276S+H358D+A364G+S388K+N474G, V45L+A117S+L149R+S214Q+V258P+A276S+S282N+H358D+A364G+S388K, V45L+A117S+L149R+S214Q+V258P+A276S+H358D+A364G+S388K+Q496Q, V45L+A117S+L149R+S214Q+V258P+A276S+A364G+S388K+S389V+D470T, V45L+A117S+L149R+S214Q+V258P+A276S+H358D+A364G+S388K+S501E, V45L+A117S+L149R+S214Q+V258P+A276S+H358D+A364G+S388K+N476N, V45L+A117S+L149R+S214Q+V258P+A276S+H358D+A364G+S388K+S473K, V45L+A117S+L149R+G187L+S214Q+V258P+A276S+H358D+A364G+S388K, V45L+A117S+L149R+S214Q+V258P+A276S+H358D+A364G+S388K+Q496E, A25W+V45L+A117S+L149R+S214Q+V258P+A276S+H358D+A364G+S388K, V45L+A117S+L149R+N168G+S214Q+V258P+A276S+H358D+A364G+S388K, V45L+A117S+L149R+S214Q+V258P+A276S+H358D+A364G+S388K+S389H, V45L+A117S+L149R+S214Q+V258P+A276S+A364G+S388K+D470T+T480L, V45L+A117S+L149R+S214Q+V258P+A276S+H358D+A364G+Q385C+S388K, V45L+S92D+A117S+L149R+S214Q+V258P+A276S+H358D+A364G+S388K, V45L+A117S+L149R+A170S+S214Q+V258P+A276S+H358D+A364G+S388K, V45L+A117S+L149R+S214Q+V258P+A276S+H358D+A364G+S388K+Y391V, V45L+A117S+L149R+S214Q+V258P+A276S+H358D+A364G+S388K+T480N, D9E+V45L+A117S+L149R+S214Q+V258P+A276S+H358D+A364G+S388K, V45L+A117S+L149R+S214Q+H219I+V258P+A276S+H358D+A364G+S388K, V45L+A117S+L149R+S214Q+V258P+A276S+H358D+A364G+S388K+A499L, V45L+A117S+L149R+S214Q+T215V+V258P+A276S+H358D+A364G+S388K, V45L+A117S+L149R+S214Q+H219V+V258P+A276S+H358D+A364G+S388K, V45L+A117S+L149R+S214Q+V258P+A276S+H358D+A364G+S388K+S389V, V45L+A117S+L149R+S214Q+V258P+A276S+H358D+A364G+S388K+V393H, V45L+A117S+N127D+L149R+S214Q+V258P+A276S+H358D+A364G+S388K, V45L+A117S+L149R+S214Q+V258P+A276S+H358D+A364G+S388K+A472L, V45L+A117S+L149R+T211H+S214Q+V258P+A276S+H358D+A364G+S388K, V45L+A117S+L149R+S214Q+V258P+A276S+H358D+A364G+S388K+T480L, V45L+A117S+L149R+S214Q+V258P+A276S+H358D+A364G+S388K+V393R, V45L+A117S+L149R+S214Q+V258P+A276S+A286S+H358D+A364G+S388K, V45L+A117S+L149R+S214Q+V258P+A276S+N355G+H358D+A364G+S388K, V45L+A117S+L149R+S214Q+V258P+A276S+N355G+A364G+S388K+D470T, V45L+A117S+L149R+S214Q+V258P+A276S+A286E+H358D+A364G+S388K, V45L+A117S+L149R+S214Q+V258P+A276S+H358D+A364G+Y386G+S388K, and V45L+A117S+L149R+S214Q+V258P+A276S+A364G+S388K+D470T+S501E of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+A79C+A117S+L149R+S214Q+V258P+A276S+A364G+S388K+D470T+R483V, V45L+A79C+A117S+L149R+S214Q+V258P+A276S+H358D+A364G+S388K+D470T, V45L+A117S+L149R+S214E+V258P+A276S+H358D+A364G+S388K+D470T+R483V, V45L+A117S+L149R+S214Q+V258P+A276S+H358D+A364G+S388K+D470T+R483V, V45L+A117S+L149R+S214E+V258P+A276S+H358D+A364G+S388K+D470T+L530R, V45L+A117S+L149R+S214Q+V258P+A276Q+H358D+A364G+S388K+D470T+R483V, V45L+A117S+L149R+S214Q+V258P+A276Q+H358D+A364G+S388K+D470T+L530R, V45L+A117S+L149R+S214Q+V258P+A276S+H358D+A364G+S388K+D470T+L530R, V45L+A117S+L149R+S214Q+V258P+A276S+H358D+A364G+S388K+D470A+R483V, V45N+A117S+L149R+S214Q+V258P+A276S+H358D+A364G+S388K+D470T+R483V, V45L+A117S+L149R+S214Q+V258P+A276S+H358D+A364G+S388H+D470T+L530R, V45N+A117S+L149R+S214Q+V258P+A276S+H358D+A364G+S388K+D470T+L530R, V45L+A117S+L149R+S214Q+V258P+A276S+H358D+A364G+S388A+D470T+L530R, V45L+A117S+L149R+S214Q+V258P+A276S+H358D+A364G+S388A+D470T+R483V, V45L+A117S+L149R+S214Q+V258P+A276S+H358D+A364G+S388L+D470T+R483V, V45L+Q61K+A117S+L149R+S214Q+V258P+A276S+H358D+A364G+S388K+D470T, V45L+A117S+L149R+S214Q+V258P+A276S+A364G+S388K+G394A+D470T+R483V, V45L+A117S+L149R+S214Q+V258P+A276S+H358D+A364G+S388K+D470T+L478N, V45L+A117S+L149R+S214Q+V258P+A276S+L281V+H358D+A364G+S388K+D470T, V45L+A117S+L149R+S214Q+V258P+A276S+H358D+A364G+T366I+S388K+D470T, V45L+A117S+L149R+S214Q+V258P+A276S+H358D+A364G+S388K+D470T+S473G, V45L+A117S+L149R+S214Q+V258P+A276S+H358D+A364G+S388K+D470T+L478P, V45L+A117S+L149R+S214Q+V258P+A276S+H358D+A364G+S388K+G394D+D470T, V45L+A117S+L149R+S214Q+V258P+A276S+A364G+Q384A+S388K+D470T+R483V, V45L+A117S+L149R+S214Q+V258P+A276S+H358D+A364G+S388K+D470T+N529T, V45L+A117S+L149R+S214Q+V258P+A276S+H358D+A364G+S388K+G394Q+D470T, V45L+A117S+L149R+G187L+S214Q+V258P+A276S+H358D+A364G+S388K+D470T, V45L+A117S+L149R+S214Q+V258P+A276S+Q314A+H358D+A364G+S388K+D470T, V45L+A117S+L149R+S214Q+V258P+A276S+V350L+H358D+A364G+S388K+D470T, V45L+A117S+L149R+G187K+S214Q+V258P+A276S+H358D+A364G+S388K+D470T, V45L+A117S+L149R+S214Q+W250Y+V258P+A276S+H358D+A364G+S388K+D470T, V45L+A117S+L149R+S214Q+V258P+A276S+A286E+H358D+A364G+S388K+D470T, V45L+A117S+L149R+S214Q+V258P+A276S+H358D+A364G+Q384A+S388K+D470T, V45L+A117S+L149R+S214Q+V258P+A276S+H358D+A364G+S388K+D470T+S528H, V45L+A117S+L149R+S214Q+V258P+A276S+H358D+A364G+S388K+D470T+Q496R, V45L+A117S+L149R+N163H+S214Q+V258P+A276S+H358D+A364G+S388K+D470T, V45L+A117S+L149R+S214Q+V258P+A276S+S282N+H358D+A364G+S388K+D470T, D11E+V45L+A117S+L149R+S214Q+V258P+A276S+H358D+A364G+S388K+D470T, V45L+A117S+L149R+S214Q+V258P+A276S+H358D+A364G+T366V+S388K+D470T, V45L+A117S+L149R+S214Q+V258P+A276S+H358D+A364G+S388K+D470T+S473K, V45L+A117S+L149R+S214Q+V258P+A276S+H358D+A364G+S388R+D470T+R483V, V45L+A117S+L149R+S214Q+V258P+A276S+Q314A+A364G+S388K+D470T+R483V, V45L+S92D+A117S+L149R+S214Q+V258P+A276S+H358D+A364G+S388K+D470T, V45L+A117S+L149R+S214Q+V258P+N260H+A276S+H358D+A364G+S388K+D470T, V45L+A117S+L149R+G187S+S214Q+V258P+A276S+H358D+A364G+S388K+D470T, V45L+A117S+L149R+S214Q+V258P+A276S+H358D+A364G+S388K+D470T+T477K, V45L+A117S+L149R+S214Q+V258P+A276S+H358D+A364G+S388K+D470T+N474G, V45L+A117S+L149R+S214Q+V258P+A276S+H358D+A364G+S388K+N395D+D470T, V45L+A117S+L149R+S214Q+V258P+A276S+H358D+A364G+S388K+D470T+S501N, V45L+A117S+N127D+L149R+S214Q+V258P+A276S+H358D+A364G+S388K+D470T, V45L+A117S+L149R+S214Q+V258P+A276S+H358D+A364G+S388K+V393R+D470T, V45L+A117S+L149R+S173Y+S214Q+V258P+A276S+H358D+A364G+S388K+D470T, V45L+A117S+L149R+S214Q+V258P+A276S+H358D+A364G+S388K+D470T+T480N, V45L+A117S+L149R+N168G+S214Q+V258P+A276S+H358D+A364G+S388K+D470T, V45L+Q61K+A117S+L149R+S214Q+V258P+A276S+A364G+S388K+D470T+R483V, V45L+D113T+A117S+L149R+S214Q+V258P+A276S+H358D+A364G+S388K+D470T, V45L+A117S+L149R+S214Q+V258P+A276S+A364G+T366V+S388K+D470T+R483V, V45L+A117S+L149R+S214Q+V258P+A276S+H358T+A364G+S388K+D470T+R483V, V45L+A117S+L149R+S214Q+V258P+A276S+A364G+S388K+V393R+D470T+R483V, V45L+A117S+L149R+S214Q+V258P+A276S+H358D+A364G+S388K+D470T+Q496Q, V45L+A117S+L149R+S214Q+V258P+A276S+A364G+S388K+G394Q+D470T+R483V, V45L+A117S+L149R+S214Q+V258P+A276S+A364G+T366I+S388K+D470T+R483V, V45L+A117S+L149R+S214Q+V258P+A276S+H358D+A364G+S388K+D470T+L484S, V45L+A117S+L149R+S214Q+V258P+A276S+H358D+A364G+S388K+D470T+A499L, V45L+A117S+L149R+S214Q+V258P+A276S+H358T+A364G+S388K+D470T+L530R, V45L+A117S+L149R+S214Q+V258P+A276S+H358D+A364G+S388K+D470T+V498I, V45L+T70R+A117S+L149R+S214Q+V258P+A276S+A364G+S388K+D470T+R483V, V45L+A117S+L149R+S214Q+V258P+A276S+A364G+S388K+D470T+N476D+R483V, D9E+V45L+A117S+L149R+S214Q+V258P+A276S+A364G+S388K+D470T+R483V, V45L+A117S+L149R+S214Q+V258P+A276S+H358D+A364G+S388K+D470T+S501E, V45L+A117S+L149R+S214Q+V258P+A276S+H358D+A364G+S388K+D470T+S481A, V45L+A117S+L149R+S214Q+H219V+V258P+A276S+H358D+A364G+S388K+D470T, V45L+A117S+L149R+S214Q+V258P+A276S+H358D+A364G+S388K+D470T+S481S, V45L+A117S+L149R+G187K+S214Q+V258P+A276S+A364G+S388K+D470T+R483V, V45L+A117S+L149R+S214Q+V258P+A276S+A364G+S388K+N395D+D470T+R483V, V45L+A117S+L149R+G187L+S214Q+V258P+A276S+A364G+S388K+D470T+R483V, V45L+A117S+L149R+S214Q+V258P+A276S+H358D+A364G+S388K+D470T+V497P, V45L+A117S+L149R+S214Q+V258P+A276S+H358D+A364G+S388K+D470T+N527H, V45L+A117S+L149R+S214Q+V258P+A276S+A364G+S388K+D470T+R483V+Q496Q, V45L+A117S+L149R+N168G+S214Q+V258P+A276S+A364G+S388K+D470T+R483V, V45L+A117S+L149R+S214Q+V258P+A276S+A364G+S388K+D470T+N476N+R483V, V45L+A117S+L149R+S214Q+V258P+A276S+A364G+S388K+D470T+T477V+R483V, V45L+A117S+L149R+S214Q+V258P+A276S+V350L+A364G+S388K+D470T+R483V, V45L+A117S+L149R+S214Q+V258P+A276S+A364G+S388K+D470T+L478N+R483V, M28P+V45L+A117S+L149R+S214Q+V258P+A276S+H358D+A364G+S388K+D470T, V45L+A117S+L149R+S214Q+V258P+A276S+A364G+S388K+V393H+D470T+R483V, V45L+Q61R+A117S+L149R+S214Q+V258P+A276S+A364G+S388K+D470T+R483V, V45L+A117S+L149R+S214Q+V258P+A276S+A364G+S388K+D470T+R483V+V497P, V45L+A117S+L149R+S214Q+V258P+A276S+A364G+S388K+D470T+R483V+V498I, V45L+A117S+L149R+S214Q+V258P+A276S+A286E+A364G+S388K+D470T+R483V, V45L+A117S+L149R+S214Q+V258P+A276S+A364G+S388K+D470T+T477K+R483V, V45L+A117S+L149R+S214Q+V258P+A276S+A364G+S388K+D470T+R483V+Q496E, V45L+A117S+L149R+S214Q+V258P+A276S+A364G+S388K+D470T+R483V+Q496R, V45L+A117S+L149R+S214Q+V258P+A276S+A364G+S388K+D470T+R483V+A499L, V45L+A117S+L149R+S214Q+T215I+V258P+A276S+H358D+A364G+S388K+D470T, V45L+A117S+L149R+S214Q+V258P+A276S+A364G+S388K+Y391V+D470T+R483V, V45L+A117S+L149R+S214Q+V258P+A276S+H358D+A364G+S388K+D470T+N476D, V45L+A117S+L149R+S214Q+V258P+A276S+H358D+A364G+S388K+V393H+D470T, V45L+A117S+L149R+S214Q+V258P+A276S+A286S+A364G+S388K+D470T+R483V, V45L+A117S+L149R+S214Q+V258P+A276S+A364G+S388K+D470T+R483V+S501E, V45L+A117S+L149R+S214Q+V258P+A276S+A364G+S388K+D470T+R483V+N529T, V45L+A117S+L149R+S214Q+V258P+A276S+A364G+S388K+D470T+R483V+S501N, V45L+A117S+L149R+S214Q+V258P+A276S+A364G+S388K+D470T+S481S+R483V, V45L+A117S+L149R+S214Q+V258P+A276S+A364G+S388K+D470T+L478P+R483V, D11E+V45L+A117S+L149R+S214Q+V258P+A276S+A364G+S388K+D470T+R483V, V45L+A117S+L149R+S214Q+V258P+A276S+H358D+A364G+S388K+D470T+T477V, V45L+A117S+L149R+T211H+S214Q+V258P+A276S+H358D+A364G+S388K+D470T, V45L+A117S+L149R+S214Y+V258P+A276S+H358D+A364G+S388K+D470T+R483V, V45L+A117S+L149R+S214Q+W250Y+V258P+A276S+A364G+S388K+D470T+R483V, V45L+S92D+A117S+L149R+S214Q+V258P+A276S+A364G+S388K+D470T+R483V, V45L+A117S+L149R+S214Q+V258P+A276S+A364G+S388K+D470T+R483V+N527H, V45L+A117S+L149R+S214Q+V258P+A276S+H358D+A364G+Q385C+S388K+D470T, V45L+A117S+L149R+S173Y+S214Q+V258P+A276S+A364G+S388K+D470T+R483V, V45L+A117S+L149R+S214Q+V258P+A276S+A364G+S388K+S389H+D470T+R483V, V45L+A117S+L149R+S214Q+H219V+V258P+A276S+A364G+S388K+D470T+R483V, V45L+A117S+L149R+S214Q+V258P+A276S+H358D+A364G+S388K+D470T+Q496E, V45L+A117S+L149R+A170S+S214Q+V258P+A276S+H358D+A364G+S388K+D470T, V45L+A117S+L149R+S214Q+V258P+A276S+H358D+A364G+S388K+S389V+D470T, V45L+T70R+A117S+L149R+S214Q+V258P+A276S+H358D+A364G+S388K+D470T, V45L+A117S+L149R+S214Q+H219I+V258P+A276S+A364G+S388K+D470T+R483V, V45L+A117S+L149R+S214Q+V258P+A276S+A364G+S388K+D470T+R483V+A499Y, V45L+A117S+L149R+S214Q+V258P+A276S+L281V+A364G+S388K+D470T+R483V, V45L+A117S+L149R+S214Q+V258P+A276S+A364G+S388K+D470T+R483V+V500T, V45L+A117S+L149R+T180Q+S214Q+V258P+A276S+H358D+A364G+S388K+D470T, V45L+A117S+L149R+S214Q+V258P+A276S+H358D+A364G+S388K+D470T+V500V, V45L+A117S+L149R+S214Q+V258P+A276S+H358D+A364G+S388K+D470T+G526T, V45L+A117S+L149R+S214Q+V258P+A276S+A286S+H358D+A364G+S388K+D470T, V45L+A117S+L149R+S214Q+V258P+A276S+H358D+A364G+S388K+D470A+L530R, V45L+A117S+L149R+S214Q+V258P+A276S+A364G+S388K+D470T+R483V+D525L, V45L+A117S+L149R+S214Q+V258P+A276S+H358D+A364G+S388K+D470T+N476N, V45L+A117S+L149R+S214Q+V258P+A276S+H358D+A364G+S388K+S389H+D470T, V45L+A117S+L149R+S214Y+V258P+A276S+H358D+A364G+S388K+D470T+L530R, A25W+V45L+A117S+L149R+S214Q+V258P+A276S+H358D+A364G+S388K+D470T, V45L+A117S+L149R+S214Q+T215V+V258P+A276S+A364G+S388K+D470T+R483V, V45L+Q61R+A117S+L149R+S214Q+V258P+A276S+H358D+A364G+S388K+D470T, V45L+A117S+L149R+S214Q+H219I+V258P+A276S+H358D+A364G+S388K+D470T, V45L+A117S+L149R+T211H+S214Q+V258P+A276S+A364G+S388K+D470T+R483V, V45L+D95N+A117S+L149R+S214Q+V258P+A276S+H358D+A364G+S388K+S473G, V45L+A117S+L149R+S214Q+V258P+A276S+H358D+A364G+S388K+D470T+G503L, V45L+A117S+L149R+S214Q+V258P+A276S+H358D+A364G+S388K+D470T+D525L, V45L+A117S+L149R+S214Q+V258P+A276S+H358D+A364G+S388K+D470T+V500T, V45L+A117S+L149R+S214Q+T215V+V258P+A276S+H358D+A364G+S388K+D470T, V45L+A117S+L149R+S214Q+V258P+A276S+H358D+A364G+S388K+D470T+T480L, V45L+A117S+L149R+S214Q+V258P+A276S+A364G+S388K+S389V+D470T+R483V, V45L+A117S+L149R+S214Q+V258P+A276S+A364G+S388K+D470T+R483V+V497E, V45L+A117S+L149R+S214Q+V258P+A276S+H358D+A364G+S388K+D470V+R483V, V45L+A117S+L149R+S214Q+V258P+A276S+H358D+A364G+S388K+D470K+R483V, V45L+A117S+L149R+S214Q+V258P+A276S+H358D+A364G+S388K+D470T+A472L, V45L+D113T+A117S+L149R+S214Q+V258P+A276S+A364G+S388K+D470T+R483V, V45L+A117S+L149R+S214Q+T215K+V258P+A276S+A364G+S388K+D470T+R483V, V45L+A117S+L149R+S214Q+V258P+A276S+A364G+S388K+D470T+R483V+V497W, V45L+A117S+L149R+S214Q+V258P+A276S+A364G+S388K+D470T+R483V+W495E, V45L+A117S+L149R+S214Q+T215K+V258P+A276S+H358D+A364G+S388K+D470T, V45L+A117S+L149R+S214Q+V258P+A276S+H358D+A364G+S388H+D470T+R483V, and V45L+A117S+L149R+S214Q+V258P+A276S+N355G+H358D+A364G+S388K+D470T of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+A79C+A117S+L149R+S214Q+V258P+A276S+H358D+A364G+S388K+D470T+L530R, V45L+A79C+A117S+L149R+S214Q+V258P+A276S+H358D+A364G+S388K+D470T+R483V, V45L+A117S+L149R+S214Q+V258P+A276S+H358D+A364G+Q385C+S388K+D470T+R483V, V45L+Q61K+A117S+L149R+S214Q+V258P+A276S+H358D+A364G+S388K+D470T+R483V, V45L+A117S+L149R+S214Q+V258P+A276S+L281V+H358D+A364G+S388K+D470T+R483V, V45L+A117S+L149R+S214Q+V258P+A276S+H358D+A364G+T366I+S388K+D470T+R483V, V45L+A117S+L149R+S214Q+V258P+A276S+H358D+A364G+Q384A+S388K+D470T+L530R, V45L+Q61K+A117S+L149R+S214Q+V258P+A276S+H358D+A364G+S388K+D470T+L530R, V45L+T70R+A117S+L149R+S214Q+V258P+A276S+H358D+A364G+S388K+D470T+R483V, V45L+A117S+L149R+S214Q+V258P+A276S+H358D+A364G+S388K+V393H+D470T+R483V, V45L+A117S+L149R+S214Q+V258P+A276S+H358D+A364G+S388K+G394A+D470T+R483V, V45L+A117S+L149R+S214Q+V258P+A276S+H358D+A364G+S388K+V393H+D470T+L530R, V45L+A117S+L149R+S214Q+V258P+A276S+A286E+H358D+A364G+S388K+D470T+R483V+D11E+V45L+A117S+L149R+S214Q+V258P+A276S+H358D+A364G+S388K+D470T+R483V, V45L+A117S+L149R+G187L+S214Q+V258P+A276S+H358D+A364G+S388K+D470T+L530R, V45L+A117S+L149R+S214Q+V258P+A276S+H358D+A364G+Q384A+S388K+D470T+R483V, V45L+A117S+L149R+G187K+S214Q+V258P+A276S+H358D+A364G+S388K+D470T+R483V, V45L+A117S+L149R+S214Q+V258P+A276S+L281V+H358D+A364G+S388K+D470T+L530R, V45L+A117S+L149R+G187L+S214Q+V258P+A276S+H358D+A364G+S388K+D470T+R483V, V45L+A117S+L149R+S214Q+V258P+A276S+H358D+A364G+S388K+G394Q+D470T+R483V, V45L+A117S+L149R+S214Q+V258P+A276S+H358D+A364G+S388K+G394A+D470T+L530R, V45L+A117S+L149R+S214Q+V258P+A276S+H358D+A364G+T366V+S388K+D470T+L530R, V45L+A117S+L149R+S214Q+V258P+A276S+H358D+A364G+S388K+G394Q+D470T+L530R, V45L+A117S+L149R+S214Q+V258P+A276S+S282N+H358D+A364G+S388K+D470T+R483V, V45L+A117S+L149R+S214Q+V258P+A276S+H358D+A364G+S388K+N395D+D470T+R483V, V45L+A117S+L149R+S214Q+V258P+A276S+H358D+A364G+S388K+D470T+N476D+R483V, V45L+A117S+L149R+S214Q+V258P+A276S+V350L+H358D+A364G+S388K+D470T+R483V, V45L+A117S+L149R+S214Q+V258P+A276S+H358D+A364G+S388K+D470T+T477K+R483V, V45L+A117S+L149R+S214Q+V258P+A276S+H358D+A364G+S388K+D470T+R483V+Q496R, V45L+A117S+L149R+S214Q+V258P+A276S+H358D+A364G+S388K+D470T+R483V+W495E, V45L+A117S+L149R+S214Q+V258P+A276S+A286E+H358D+A364G+S388K+D470T+L530R, V45L+A117S+L149R+G187K+S214Q+V258P+A276S+H358D+A364G+S388K+D470T+L530R, V45L+A117S+L149R+S214Q+V258P+A276S+H358D+A364G+S388K+Y391V+D470T+R483V, D9E+V45L+A117S+L149R+S214Q+V258P+A276S+H358D+A364G+S388K+D470T+L530R, V45L+A117S+L149R+S214Q+V258P+A276S+H358D+A364G+S388K+D470T+T477V+R483V, V45L+A117S+L149R+S214Q+V258P+A276S+H358D+A364G+S388K+D470T+N476N+R483V, V45L+A117S+L149R+S214Q+V258P+A276S+H358D+A364G+S388K+V393R+D470T+R483V, V45L+A117S+L149R+S214Q+V258P+A276S+H358D+A364G+S388K+D470T+T480L+L530R, V45L+A117S+L149R+S214Q+V258P+A276S+H358D+A364G+S388K+D470T+S481A+L530R, V45L+A117S+L149R+S214Q+W250Y+V258P+A276S+H358D+A364G+S388K+D470T+R483V, V45L+A117S+L149R+S214Q+V258P+A276S+H358D+A364G+S388K+D470T+S481S+R483V, V45L+A117S+L149R+S173Y+S214Q+V258P+A276S+H358D+A364G+S388K+D470T+R483V, V45L+A117S+L149R+T180Q+S214Q+V258P+A276S+H358D+A364G+S388K+D470T+L530R, D11E+V45L+A117S+L149R+S214Q+V258P+A276S+H358D+A364G+S388K+D470T+L530R, V45L+A117S+L149R+S214Q+V258P+A276S+H358D+A364G+S388K+D470T+N476D+L530R, V45L+A117S+L149R+S214Q+V258P+A276S+A286S+H358D+A364G+S388K+D470T+R483V, V45L+A117S+L149R+S214Q+V258P+A276S+H358D+A364G+S388K+D470T+R483V+Q496Q, V45L+A117S+L149R+S214Q+V258P+A276S+H358D+A364G+S388K+D470T+R483V+Q496E, V45L+Q61R+A117S+L149R+S214Q+V258P+A276S+H358D+A364G+S388K+D470T+L530R, V45L+A117S+L149R+S214Q+V258P+A276S+Q314A+H358D+A364G+S388K+D470T+L530R, V45L+A117S+L149R+S214Q+V258P+A276S+H358D+A364G+S388K+D470T+L478P+R483V, A25W+V45L+A117S+L149R+S214Q+V258P+A276S+H358D+A364G+S388K+D470T+R483V, V45L+A117S+L149R+T180Q+S214Q+V258P+A276S+H358D+A364G+S388K+D470T+R483V, V45L+A117S+L149R+S214Q+V258P+A276S+H358D+A364G+S388K+D470T+R483V+L484K, V45L+A117S+L149R+S214Q+V258P+A276S+H358D+A364G+S388K+D470T+N474G+L530R, V45L+A117S+L149R+S214Q+V258P+A276S+H358D+A364G+S388K+N395D+D470T+L530R, V45L+A117S+L149R+S214Q+V258P+A276S+H358D+A364G+T366V+S388K+D470T+R483V, V45L+A117S+L149R+N168G+S214Q+V258P+A276S+H358D+A364G+S388K+D470T+R483V, V45L+A117S+L149R+S214Q+V258P+A276S+H358D+A364G+S388K+D470T+R483V+L484S, V45L+A117S+L149R+S214Q+V258P+A276S+H358D+A364G+S388K+D470T+R483V+L484D, V45L+A117S+L149R+S214Q+V258P+A276S+H358D+A364G+S388K+D470T+R483V+S528H, V45L+A117S+L149R+S214Q+V258P+A276S+H358D+A364G+S388K+D470T+R483V+S501N, V45L+A117S+L149R+S173Y+S214Q+V258P+A276S+H358D+A364G+S388K+D470T+L530R, V45L+A117S+L149R+S214Q+V258P+A276S+H358D+A364G+S388K+D470T+R483V+A499L, V45L+S92D+A117S+L149R+S214Q+V258P+A276S+H358D+A364G+S388K+D470T+R483V, V45L+A117S+L149R+S214Q+V258P+A276S+Q314A+H358D+A364G+S388K+D470T+R483V, V45L+A117S+L149R+S214Q+V258P+A276S+N355G+H358D+A364G+S388K+D470T+L530R, V45L+A117S+L149R+S214Q+V258P+A276S+V350L+H358D+A364G+S388K+D470T+L530R, V45L+A117S+L149R+S214Q+V258P+A276S+H358D+A364G+S388K+D470T+S481S+L530R, V45L+A117S+L149R+S214Q+V258P+A276S+H358D+A364G+S388K+D470T+Q496E+L530R, V45L+A117S+L149R+N168G+S214Q+V258P+A276S+H358D+A364G+S388K+D470T+L530R, V45L+A117S+L149R+S214Q+V258P+A276S+H358D+A364G+S388K+D470T+V498I+L530R, V45L+A117S+L149R+S214Q+V258P+A276S+H358D+A364G+S388K+D470T+R483V+A499Y, V45L+A117S+L149R+S214Q+V258P+A276S+A286S+H358D+A364G+S388K+D470T+L530R, V45L+A117S+L149R+S214Q+V258P+A276S+H358D+A364G+S388K+D470T+S501N+L530R, V45L+A117S+L149R+S214Q+V258P+A276S+H358D+A364G+S388K+D470T+N476N+L530R, V45L+A117S+L149R+S214Q+V258P+A276S+H358D+A364G+S388K+D470T+T477V+L530R, V45L+A117S+L149R+S214Q+V258P+A276S+H358D+A364G+S388K+D470T+L478N+L530R, V45L+S92D+A117S+L149R+S214Q+V258P+A276S+H358D+A364G+S388K+D470T+L530R, V45L+A117S+L149R+S214Q+V258P+A276S+H358D+A364G+S388K+D470T+V500V+L530R, V45L+A117S+L149R+S214Q+V258P+A276S+H358D+A364G+S388K+D470T+R483V+V500V, V45L+A117S+L149R+S214Q+V258P+A276S+H358D+A364G+S388K+D470T+L478P+L530R, V45L+A117S+L149R+S214Q+V258P+A276S+H358D+A364G+S388K+D470T+R483V+S501E, V45L+A117S+L149R+S214Q+V258P+A276S+H358D+A364G+S388K+D470T+D525L+L530R, V45L+A117S+L149R+S214Q+V258P+A276S+H358D+A364G+S388K+D470T+G526T+L530R, V45L+A117S+L149R+S214Q+V258P+A276S+H358D+A364G+S388K+D470T+N527H+L530R, V45L+A117S+L149R+S214Q+V258P+A276S+H358D+A364G+S388K+D470T+T477K+L530R, M28P+V45L+A117S+L149R+S214Q+V258P+A276S+H358D+A364G+S388K+D470T+L530R, D9E+V45L+A117S+L149R+S214Q+V258P+A276S+H358D+A364G+S388K+D470T+R483V, V45L+A117S+L149R+S214Q+V258P+A276S+H358D+A364G+S388K+D470T+S501E+L530R, V45L+A117S+L149R+S214Q+W250Y+V258P+A276S+H358D+A364G+S388K+D470T+L530R, V45L+A117S+L149R+S214Q+V258P+A276S+H358D+A364G+S388K+D470T+A499L+L530R, V45L+A117S+L149R+S214Q+V258P+A276S+H358D+A364G+S388K+D470T+A472I+L530R, V45L+A117S+N127D+L149R+S214Q+V258P+A276S+H358D+A364G+S388K+D470T+R483V, V45L+A117S+L149R+S214Q+V258P+A276S+H358D+A364G+S388K+D470T+V500T+L530R, V45L+A117S+L149R+S214Q+V258P+A276S+H358D+A364G+S388K+D470T+R483V+N527H, V45L+A117S+L149R+S214Q+V258P+A276S+H358D+A364G+S388K+D470T+T480N+L530R, V45L+A117S+L149R+S214Q+V258P+A276S+H358D+A364G+S388K+D470T+R483V+V497P, V45L+A117S+L149R+S214Q+V258P+A276S+H358D+A364G+S388K+D470T+R483V+V498I, V45L+A117S+L149R+S214Q+V258P+A276S+H358D+A364G+S388K+D470T+T480N+R483V, V45L+A117S+L149R+S214Q+H219V+V258P+A276S+H358D+A364G+S388K+D470T+L530R, M28P+V45L+A117S+L149R+S214Q+V258P+A276S+H358D+A364G+S388K+D470T+R483V, V45L+A117S+L149R+A170S+S214Q+V258P+A276S+H358D+A364G+S388K+D470T+R483V, V45L+A117S+L149R+S214Q+V258P+A276S+H358D+A364G+S388K+S389H+D470T+L530R, V45L+D113T+A117S+L149R+S214Q+V258P+A276S+H358D+A364G+S388K+D470T+L530R, V45L+A117S+L149R+N163H+S214Q+V258P+A276S+H358D+A364G+S388K+D470T+R483V, V45L+A117S+L149R+T211H+S214Q+V258P+A276S+H358D+A364G+S388K+D470T+L530R, V45L+A117S+L149R+S214Q+V258P+A276S+H358D+A364G+S388K+D470T+R483V+V500T, V45L+D113T+A117S+L149R+S214Q+V258P+A276S+H358D+A364G+S388K+D470T+R483V, V45L+D95N+A117S+L149R+S214Q+V258P+A276S+H358D+A364G+S388K+G394A+D470T, V45L+A93T+A117S+L149R+S214Q+V258P+A276S+H358D+A364G+S388K+D470T+A499Y, V45L+A117S+L149R+S214Q+T215I+V258P+A276S+H358D+A364G+S388K+D470T+R483V, V45L+A117S+L149R+S214Q+H219I+V258P+A276S+H358D+A364G+S388K+D470T+R483V, V45L+A117S+L149R+S214Q+V258P+A276S+H358D+A364G+Q385C+S388K+D470T+L530R, V45L+A117S+L149R+S214Q+V258P+A276S+H358D+A364G+S388K+D470T+R483V+L530R, V45L+A117S+L149R+S214Q+V258P+A276S+H358D+A364G+S388K+D470T+R483V+G503L, V45L+A117S+L149R+S214Q+T215V+V258P+A276S+H358D+A364G+S388K+D470T+R483V, V45L+A117S+L149R+S214Q+V258P+A276S+H358D+A364G+S388K+D470T+S481A+R483V, V45L+A117S+L149R+S214Q+T215V+V258P+A276S+H358D+A364G+S388K+D470T+L530R, V45L+A117S+L149R+G187S+S214Q+V258P+A276S+H358D+A364G+S388K+D470T+L530R, V45L+A117S+L149R+T211H+S214Q+V258P+A276S+H358D+A364G+S388K+D470T+R483V, V45L+A117S+L149R+S214Q+V258P+A276S+H358D+A364G+S388K+D470T+R483V+G526T, V45L+A117S+L149R+S214Q+V258P+A276S+H358D+A364G+S388K+D470T+R483V+D525L, V45L+A117S+L149R+S214Q+V258P+A276S+H358D+A364G+S388K+D470T+S528H+L530R, V45L+A117S+L149R+S214Q+V258P+A276S+H358D+A364G+S388K+I469T+D470T+R483V, V45L+A117S+L149R+S214Q+V258P+A276S+H358D+A364G+S388K+D470T+N529T+L530R, V45L+A117S+L149R+S214Q+V258P+A276S+S282N+H358D+A364G+S388K+D470T+L530R, V45L+A117S+L149R+S214Q+V258P+A276S+H358D+A364G+S388K+D470T+G503L+L530R, V45L+A117S+L149R+S214Q+H219I+V258P+A276S+H358D+A364G+S388K+D470T+L530R, V45L+A117S+L149R+S214Q+V258P+A276S+H358D+A364G+S388K+D470T+V497P+L530R, V45L+A117S+L149R+S214Q+T215I+V258P+A276S+H358D+A364G+S388K+D470T+L530R, V45L+A117S+L149R+S214Q+V258P+A276S+H358D+A364G+S388K+S389V+D470T+R483V, V45L+A117S+L149R+S214Q+V258P+A276S+H358D+A364G+S388K+S389V+D470T+L530R, V45L+A117S+L149R+S214Q+V258P+A276S+H358D+A364G+S388K+D470T+R483V+V497D, V45L+A117S+L149R+S214Q+V258P+A276S+H358D+A364G+S388K+D470T+L478N+R483V, V45L+T70R+D95N+A117S+L149R+S214Q+V258P+A276S+L356M+H358D+A364G+S388K, V45L+A117S+L149R+S214Q+T215K+V258P+A276S+H358D+A364G+S388K+D470T+R483V, V45L+A117S+L149R+S214Q+V258P+A276S+H358D+A364G+S388K+D470T+R483V+V497W, V45L+T70R+A117S+L149R+S214Q+V258P+A276S+H358D+A364G+S388K+D470T+L530R, V45L+A117S+L149R+S214Q+T215K+V258P+A276S+H358D+A364G+S388K+D470T+L530R, V45L+Q61R+A117S+L149R+S214Q+V258P+A276S+H358D+A364G+S388K+D470T+R483V, V45L+A117S+L149R+S214Q+V258P+A276S+N355G+H358D+A364G+S388K+D470T+R483V, V45L+A117S+L149R+S214Q+V258P+A276S+H358D+A364G+S388K+D470T+R483V+V497E, V45L+A117S+L149R+N163H+S214Q+V258P+A276S+H358D+A364G+S388K+D470T+L530R, V45L+A117S+L149R+S214Q+V258P+A276S+H358D+A364G+Y386G+S388K+D470T+R483V, and V45L+A117S+L149R+S214Q+V258P+A276S+H358D+A364G+S388K+G394D+D470T+R483V of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+T70R+A117S+L149R+S214Q+V258P+A276S+L281V+H358D+A364G+S388K+G394A+D470T+R483V, V45L+T70R+A117S+L149R+S214Q+V258P+A276S+L281V+H358D+A364G+S388K+G394A+D470T+R483V, V45L+T70R+A117S+L149R+S214Q+V258P+A276S+L281V+H358D+A364G+S388K+G394A+D470T+R483V, V45L+T70R+A117S+L149R+S214Q+V258P+A276S+L281V+H358D+A364G+S388K+G394A+D470T+R483V, V45L+T70R+A117S+L149R+S214Q+V258P+A276S+L281V+H358D+A364G+S388K+G394A+D470T+R483V, V45L+T70R+A117S+L149R+S214Q+V258P+A276S+L281V+H358D+A364G+S388K+G394A+D470T+R483V, V45L+T70R+A117S+L149R+S214Q+V258P+A276S+L281V+H358D+A364G+S388K+G394A+D470T+R483V, V45L+T70R+A117S+L149R+S214Q+V258P+A276S+L281V+H358D+A364G+S388K+G394A+D470T+R483V, V45L+T70R+A117S+L149R+S214Q+V258P+A276S+L281V+H358D+A364G+S388K+G394A+D470T+R483V, V45L+T70R+A117S+L149R+S214Q+V258P+A276S+L281V+H358D+A364G+S388K+G394A+D470T+R483V, V45L+Q61K+A117S+L149R+S214Q+V258P+A276S+L281V+H358D+A364G+S388K+G394A+D470T+L530R, V45L+Q61K+A117S+L149R+S214Q+V258P+A276S+L281V+H358D+A364G+S388K+D470T+R483V+L530R, V45L+Q61K+A117S+L149R+S214Q+V258P+A276S+L281V+H358D+A364G+S388K+D470T+R483V+L530R, and V45L+T70R+A117S+L149R+S214Q+V258P+A276S+L281V+H358D+A364G+S388K+G394A+D470T+R483V of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+Q61K+A117S+L149R+S214Q+V258P+A276S+L281V+H358D+A364G+S388K+G394A+D470T+R483V+L530R, V45L+Q61K+A117S+L149R+S214Q+V258P+A276S+L281V+H358D+A364G+S388K+G394A+D470T+R483V+L530R, V45L+Q61K+A117S+L149R+S214Q+V258P+A276S+L281V+H358D+A364G+S388K+G394A+D470T+R483V+L530R, V45L+Q61K+A117S+L149R+S214Q+V258P+A276S+L281V+H358D+A364G+S388K+G394A+D470T+R483V+L530R, V45L+A117S+L149R+G187K+S214Q+V258P+A276S+A286E+H358D+A364G+Q384A+S388K+V393H+D470T+R483V, V45L+A117S+L149R+G187K+S214Q+V258P+A276S+A286E+H358D+A364G+Q384A+S388K+N395D+D470T+R483V, V45L+A117S+L149R+G187K+S214E+T215I+V258P+A276S+A286E+H358D+A364G+S388K+V393H+D470T+R483V, V45L+A117S+L149R+G187K+S214E+T215I+V258P+A276S+A286E+H358D+A364G+S388K+N395D+D470T+R483V, V45L+T70R+A117S+L149R+S214Q+V258P+A276S+L281V+H358D+A364G+S388K+G394A+C468V+D470T+R483V, V45L+T70R+A117S+L149R+S214Q+V258P+A276S+L281V+H358D+A364G+S388K+G394A+C468A+D470T+R483V, V45L+T70R+A117S+L149R+S214Q+C243V+V258P+A276S+L281V+H358D+A364G+S388K+G394A+D470T+R483V, V45L+T70R+A117S+L149R+S214Q+V258P+A276S+L281V+H358D+A364G+S388K+G394A+C468L+D470T+R483V, V45L+T70R+A117S+L149R+S214Q+V258P+A276S+L281V+H358D+A364G+C375V+S388K+G394A+D470T+R483V, V45L+T70R+A117S+L149R+S214Q+V258P+A276S+L281V+H358D+A364G+S388K+G394A+C468S+D470T+R483V, V45L+C60L+T70R+A117S+L149R+S214Q+V258P+A276S+L281V+H358D+A364G+S388K+G394A+D470T+R483V, V45L+C69L+T70R+A117S+L149R+S214Q+V258P+A276S+L281V+H358D+A364G+S388K+G394A+D470T+R483V, V45L+C60A+T70R+A117S+L149R+S214Q+V258P+A276S+L281V+H358D+A364G+S388K+G394A+D470T+R483V, V45L+T70R+A117S+L149R+S214Q+C243L+V258P+A276S+L281V+H358D+A364G+S388K+G394A+D470T+R483V, V45L+C60S+T70R+A117S+L149R+S214Q+V258P+A276S+L281V+H358D+A364G+S388K+G394A+D470T+R483V, V45L+C69A+T70R+A117S+L149R+S214Q+V258P+A276S+L281V+H358D+A364G+S388K+G394A+D470T+R483V, V45L+T70R+A117S+L149R+C161S+S214Q+V258P+A276S+L281V+H358D+A364G+S388K+G394A+D470T+R483V, V45L+C69V+T70R+A117S+L149R+S214Q+V258P+A276S+L281V+H358D+A364G+S388K+G394A+D470T+R483V, V45L+C60V+T70R+A117S+L149R+S214Q+V258P+A276S+L281V+H358D+A364G+S388K+G394A+D470T+R483V, V45L+T70R+A117S+L149R+S214Q+V258P+A276S+L281V+H358D+A364G+C375A+S388K+G394A+D470T+R483V, V45L+T70R+A117S+L149R+S214Q+V258P+A276S+L281V+H358D+A364G+C375S+S388K+G394A+D470T+R483V, V45L+T70R+A117S+L149R+S214Q+V258P+A276S+L281V+H358D+A364G+S388K+G394A+D470T+R483V+C540A, V45L+C69S+T70R+A117S+L149R+S214Q+V258P+A276S+L281V+H358D+A364G+S388K+G394A+D470T+R483V, V45L+T70R+A117S+L149R+S214Q+V258P+A276S+L281V+H358D+A364G+C375L+S388K+G394A+D470T+R483V, V45L+T70R+A117S+L149R+S214Q+V258P+A276S+L281V+H358D+A364G+S388K+G394A+D470T+R483V+C540L, V45L+T70R+A117S+L149R+S214Q+V258P+A276S+L281V+H358D+A364G+S388K+G394A+D470T+R483V+C522S, V45L+T70R+A117S+L149R+S214Q+V258P+A276S+L281V+H358D+A364G+S388K+G394A+D470T+R483V+C540S, V45L+T70R+A117S+L149R+S214Q+V258P+A276S+L281V+H358D+A364G+S388K+G394A+C396S+D470T+R483V, V45L+T70R+A117S+L149R+S214Q+V258P+A276S+L281V+H358D+A364G+S388K+G394A+C396A+D470T+R483V, C40L, V45L+T70R+A117S+L149R+S214Q+V258P+A276S+L281V+H358D+A364G+S388K+G394A+D470T+R483V, C40A, V45L+T70R+A117S+L149R+S214Q+V258P+A276S+L281V+H358D+A364G+S388K+G394A+D470T+R483V+C40S, V45L+T70R+A117S+L149R+S214Q+V258P+A276S+L281V+H358D+A364G+S388K+G394A+D470T+R483V, C40V, V45L+T70R+A117S+L149R+S214Q+V258P+A276S+L281V+H358D+A364G+S388K+G394A+D470T+R483V, V45L+T70R+A117S+L149R+S214Q+V258P+A276S+L281V+H358D+A364G+S388K+G394A+C396V+D470T+R483V, V45L+T70R+A117S+L149R+C161V+S214Q+V258P+A276S+L281V+H358D+A364G+S388K+G394A+D470T+R483V, V45L+T70R+A117S+L149R+S214Q+V258P+A276S+L281V+H358D+A364G+S388K+G394A+C396L+D470T+R483V, V45L+T70R+A117S+L149R+C161L+S214Q+V258P+A276S+L281V+H358D+A364G+S388K+G394A+D470T+R483V, V45L+T70R+A117S+L149R+S214Q+V258P+A276S+L281V+H358D+A364G+S388K+G394A+D470T+R483V+C522A, V45L+T70R+A117S+L149R+S214Q+C243S+V258P+A276S+L281V+H358D+A364G+S388K+G394A+D470T+R483V, V45L+T70R+A117S+L149R+S214Q+V258P+A276S+L281V+H358D+A364G+S388K+G394A+D470T+R483V+C522V, and V45L+T70R+A117S+L149R+S214Q+V258P+A276S+L281V+H358D+A364G+S388K+G394A+D470T+R483V+C540V of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+Q61K+T70R+A117S+L149R+S214Q+V258P+A276S+L281V+H358D+A364G+Q384A+S388K+G394Q+D470T+R483V, V45L+Q61K+T70R+A117S+L149R+S214Q+V258P+A276S+L281V+H358D+A364G+Q384A+S388K+G394A+D470T+R483V, V45L+Q61K+T70R+A117S+L149R+S214Q+V258P+A276S+L281V+H358D+A364G+Q384A+S388K+V393H+D470T+R483V, V45L+Q61K+T70R+A117S+L149R+S214Q+V258P+A276S+L281V+H358D+A364G+Q384A+S388K+V393H+D470T+R483V, V45L+Q61K+T70R+A117S+L149R+S214Q+V258P+A276S+L281V+H358D+A364G+Q384A+S388K+G394A+D470T+R483V, V45L+Q61K+T70R+A117S+L149R+S214Q+V258P+A276S+L281V+H358D+A364G+Q384A+S388K+V393H+D470T+R483V, V45L+Q61K+T70R+A117S+L149R+S214Q+V258P+A276S+L281V+H358D+A364G+Q384A+S388K+G394A+D470T+R483V, V45L+Q61K+T70R+A117S+L149R+S214Q+V258P+A276S+L281V+H358D+A364G+Q384A+S388K+V393H+D470T+R483V, and V45L+Q61K+T70R+A117S+L149R+S214Q+V258P+A276S+L281V+H358D+A364G+Q384A+S388K+G394A+D470T+R483V of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, a variant comprises substitutions at positions corresponding to positions selected from a group consisting of: V45L+Q61K+T70R+A117S+L149R+S214Q+V258P+A276S+L281V+A286E+H358D+A364G+Q384A+S388K+V393H+D470T+R483V, and V45L+Q61K+T70R+A117S+L149R+S214Q+V258P+A276S+L281V+A286E+H358D+A364G+Q384A+S388K+V393H+D470T+R483V of SEQ ID NO: 1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In one aspect, the xylanase variants of the present invention have an improved property relative to the parent polypeptide, wherein the improved property is selected from the group consisting of increased catalytic efficiency, increased catalytic rate, increased chemical stability, increased oxidation stability, increased pH activity, increased pH stability, increased specific activity, increased stability under storage conditions, increased substrate binding, increased substrate cleavage, increased substrate specificity, increased substrate stability, increased surface properties, increased thermal activity, and increased thermostability.

In one aspect, the xylanase variants of the present invention have improved property relative to said parent polypeptide.

In one aspect, the xylanase variants of the present invention have improved property relative to said parent polypeptide and wherein said improved property is increased thermostability.

In one aspect, the xylanase variant has improved (increased) thermostability relative to the parent xylanase.

In one aspect, the xylanase variant has improved (increased) thermostability relative to SEQ ID NO: 1.

In one aspect, the xylanase variant has increased thermostability measured as increased melting temperature using TSA.

In one aspect, the xylanase variant has increased thermostability measured as increased melting temperature using TSA relative to SEQ ID NO: 1 of at least 1° C., at least 1.5° C., at least 2° C., at least 2.5° C., at least 3° C., at least 3.5° C., at least 4.0° C., at least 4.5° C. or at least 5° C.

In one aspect, the xylanase variant has increased thermostability measured as increased melting temperature using n-DSF.

In one aspect, the xylanase variant has increased thermostability measured as increased melting temperature using n-DSF relative to SEQ ID NO: 1 of at least 1° C., at least 1.5° C., at least 2° C., at least 2.5° C., at least 3° C., at least 3.5° C., at least 4.0° C., at least 4.5° C. or at least 5° C.

In one aspect, the present invention relates to xylanase variants comprises at least one of the following substitutions or combinations of substitutions:

V45L+Q61K+T70R+A117S+L149R+S214Q+V258P+A276S+L281V+A286E+H358D+A364G+Q384A+S388K+V393H+D470T+R483V;

V45L+Q61K+T70R+A117S+L149R+S214Q+V258P+A276S+L281V+H358D+A364G+Q384A+S388K+G394Q+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+A286E+H358D+A364G+Q384A+S388K+V393H+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+A79C+A117S+L149R+S214Q+V258P+A276S+H358D+A364G+S388K+D470T+L530R;

V45L+A79C+A117S+L149R+S214Q+V258P+A276S+H358D+A364G+S388K+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+Q61K+A117S+L149R+S214Q+V258P+A276S+L281V+H358D+A364G+S388K+G394A+D470T+R483V+L530R;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+Q61K+A117S+L149R+S214Q+V258P+A276S+L281V+H358D+A364G+S388K+G394A+D470T+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+D470T+R483V+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+D470T+R483V+L530R;

V45L+A117S+L149R+G187K+S214Q+V258P+A276S+A286E+H358D+A364G+Q384A+S388K+V393H+D470T+R483V;

V45L+A117S+L149R+G187K+S214Q+V258P+A276S+A286E+H358D+A364G+Q384A+S388K+N395D+D470T+R483V;

V45L+A117S+L149R+G187K+S214E+T215I+V258P+A276S+A286E+H358D+A364G+S388K+V393H+D470T+R483V;

V45L+A117S+L149R+G187K+S214E+T215I+V258P+A276S+A286E+H358D+A364G+S388K+N395D+D470T+R483V;

V45L+A117S+L149R+S214Q+V258P+A276S+H358D+A364G+Q385C+S388K+D470T+R483V;

V45L+Q61K+A117S+L149R+S214Q+V258P+A276S+H358D+A364G+S388K+D470T+R483V;

V45L+A79C+A117S+L149R+S214Q+V258P+A276S+A364G+S388K+D470T+R483V;

V45L+A117S+L149R+S214Q+V258P+A276S+L281V+H358D+A364G+S388K+D470T+R483V;

V45L+A117S+L149R+S214Q+V258P+A276S+H358D+A364G+T366I+S388K+D470T+R483V;

V45L+T70R+A117S+L149R+S214Q+V258P+A276S+L281V+H358D+A364G+S388K+G394A+D470T+R483V;

V45L+A117S+L149R+S214Q+V258P+A276S+H358D+A364G+Q384A+S388K+D470T+L530R;

V45L+Q61K+A117S+L149R+S214Q+V258P+A276S+H358D+A364G+S388K+D470T+L530R;

V45L+T70R+A117S+L149R+S214Q+V258P+A276S+H358D+A364G+S388K+D470T+R483V;

V45L+A117S+L149R+S214Q+V258P+A276S+H358D+A364G+S388K+V393H+D470T+R483V;

V45L+A117S+L149R+S214Q+V258P+A276S+H358D+A364G+S388K+G394A+D470T+R483V;

V45L+A117S+L149R+S214Q+V258P+A276S+H358D+A364G+S388K+V393H+D470T+L530R;

V45L+A117S+L149R+S214Q+V258P+A276S+A286E+H358D+A364G+S388K+D470T+R483V; D11E+V45L+A117S+L149R+S214Q+V258P+A276S+H358D+A364G+S388K+D470T+R483V;

V45L+T70R+A117S+L149R+S214Q+V258P+A276S+L281V+H358D+A364G+S388K+G394A+C468V+D470T+R483V;

V45L+A79C+A117S+L149R+S214Q+V258P+A276S+H358D+A364G+S388K+D470T V45L+A79C+L149R+S214E+V258P+A276S+A364G+S388K;

V45L+A117S+L149R+G187L+S214Q+V258P+A276S+H358D+A364G+S388K+D470T+L530R;

V45L+A117S+L149R+S214Q+V258P+A276S+H358D+A364G+Q384A+S388K+D470T+R483V;

V45L+A117S+L149R+G187K+S214Q+V258P+A276S+H358D+A364G+S388K+D470T+R483V;

V45L+A117S+L149R+S214Q+V258P+A276S+L281V+H358D+A364G+S388K+D470T+L530R;

V45L+A117S+L149R+S214E+V258P+A276S+H358D+A364G+S388K+D470T+R483V;

V45L+A117S+L149R+G187L+S214Q+V258P+A276S+H358D+A364G+S388K+D470T+R483V;

V45L+A117S+L149R+S214Q+V258P+A276S+H358D+A364G+S388K+G394Q+D470T+R483V;

V45L+A117S+L149R+S214Q+V258P+A276S+H358D+A364G+S388K+G394A+D470T+L530R;

V45L+A117S+L149R+S214Q+V258P+A276S+H358D+A364G+T366V+S388K+D470T+L530R;

V45L+A117S+L149R+S214Q+V258P+A276S+H358D+A364G+S388K+G394Q+D470T+L530R;

V45L+A117S+L149R+S214Q+V258P+A276S+S282N+H358D+A364G+S388K+D470T+R483V;

V45L+A117S+L149R+S214Q+V258P+A276S+H358D+A364G+S388K+N395D+D470T+R483V;

V45L+A117S+L149R+S214Q+V258P+A276S+H358D+A364G+S388K+D470T+N476D+R483V;

V45L+A117S+L149R+S214Q+V258P+A276S+V350L+H358D+A364G+S388K+D470T+R483V;

V45L+A117S+L149R+S214Q+V258P+A276S+H358D+A364G+S388K+D470T+T477K+R483V;

V45L+A117S+L149R+S214Q+V258P+A276S+H358D+A364G+S388K+D470T+R483V+Q496R;

V45L+T70R+A117S+L149R+S214Q+V258P+A276S+L281V+H358D+A364G+S388K+G394A+C468A+D470T+R483V;

V45L+A117S+L149R+S214Q+V258P+A276S+H358D+A364G+S388K+D470T+R483V+W495E;

V45L+A117S+L149R+S214Q+V258P+A276S+A286E+H358D+A364G+S388K+D470T+L530R;

V45L+A117S+L149R+G187K+S214Q+V258P+A276S+H358D+A364G+S388K+D470T+L530R;

V45L+A117S+L149R+S214Q+V258P+A276S+H358D+A364G+S388K+Y391V+D470T+R483V;

V45L+A117S+L149R+S214Q+V258P+A276S+H358D+A364G+S388K+D470T+R483V; D9E+V45L+A117S+L149R+S214Q+V258P+A276S+H358D+A364G+S388K+D470T+L530R;

V45L+A117S+L149R+S214Q+V258P+A276S+H358D+A364G+S388K+D470T+T477V+R483V;

V45L+A117S+L149R+S214Q+V258P+A276S+H358D+A364G+S388K+D470T+N476N+R483V;

V45L+A117S+L149R+S214Q+V258P+A276S+H358D+A364G+S388K+V393R+D470T+R483V;

V45L+A117S+L149R+S214Q+V258P+A276S+H358D+A364G+S388K+D470T+T480L+L530R;

V45L+A117S+L149R+S214Q+V258P+A276S+H358D+A364G+S388K+D470T+S481A+L530R;

V45L+A117S+L149R+S214Q+W250Y+V258P+A276S+H358D+A364G+S388K+D470T+R483V;

V45L+A117S+L149R+S214Q+V258P+H358D+A364G+S388K+D470T+S481S+R483V;

V45L+A117S+L149R+S173Y+S214Q+V258P+A276S+H358D+A364G+S388K+D470T+R483V;

V45L+T70R+A117S+L149R+S214Q+C243V+V258P+A276S+L281V+H358D+A364G+S388K+G394A+D470T+R483V;

V45L+A117S+L149R+T180Q+S214Q+V258P+A276S+H358D+A364G+S388K+D470T+L530R; D11E+V45L+A117S+L149R+S214Q+V258P+A276S+H358D+A364G+S388K+D470T+L530R;

V45L+A117S+L149R+S214E+V258P+A276S+H358D+A364G+S388K+D470T+L530R;

V45L+A117S+L149R+S214Q+V258P+A276S+H358D+A364G+S388K+D470T+N476D+L530R;

V45L+A117S+L149R+S214Q+V258P+A276S+A286S+H358D+A364G+S388K+D470T+R483V;

V45L+A117S+L149R+S214Q+V258P+A276S+H358D+A364G+S388K+D470T+R483V+Q496Q;

V45L+A117S+L149R+S214Q+V258P+A276Q+H358D+A364G+S388K+D470T+R483V;

V45L+A117S+L149R+S214Q+V258P+A276S+H358D+A364G+S388K+D470T+R483V+Q496E;

V45L+Q61R+A117S+L149R+S214Q+V258P+A276S+H358D+A364G+S388K+D470T+L530R;

V45L+A117S+L149R+S214Q+V258P+A276S+Q314A+H358D+A364G+S388K+D470T+L530R;

V45L+A117S+L149R+S214Q+V258P+A276Q+H358D+A364G+S388K+D470T+L530R;

V45L+A117S+L149R+S214Q+V258P+A276S+H358D+A364G+S388K+D470T+L478P+R483V; A25W+V45L+A117S+L149R+S214Q+V258P+A276S+H358D+A364G+S388K+D470T+R483V;

V45L+A117S+L149R+T180Q+S214Q+V258P+A276S+H358D+A364G+S388K+D470T+R483V;

V45L+A117S+L149R+S214Q+V258P+A276S+H358D+A364G+S388K+D470T+R483V+L484K

V45L+A117S+L149R+S214Q+V258P+A276S+H358D+A364G+S388K+D470T+N474G+L530R;

V45L+A117S+L149R+S214Q+V258P+A276S+H358D+A364G+S388K+N395D+D470T+L530R;

V45L+A117S+L149R+S214Q+V258P+A276S+H358D+A364G+T366V+S388K+D470T+R483V;

V45L+A117S+L149R+N168G+S214Q+V258P+A276S+H358D+A364G+S388K+D470T+R483V;

V45L+A117S+L149R+S214Q+V258P+A276S+H358D+A364G+S388K+D470T+R483V+L484S;

V45L+A117S+L149R+S214Q+V258P+A276S+H358D+A364G+S388K+D470T+L530R;

V45L+A117S+L149R+S214Q+V258P+A276S+H358D+A364G+S388K+D470T+R483V+L484D;

V45L+A117S+L149R+S214Q+V258P+A276S+H358D+A364G+S388K+D470T+R483V+S528H;

V45L+A117S+L149R+S214Q+V258P+A276S+H358D+A364G+S388K+D470T+R483V+S501N;

V45L+A117S+L149R+S173Y+S214Q+V258P+A276S+H358D+A364G+S388K+D470T+L530R;

V45L+A117S+L149R+S214Q+V258P+A276S+H358D+A364G+S388K+D470T+R483V+A499L;

V45L+S92D+A117S+L149R+S214Q+V258P+A276S+H358D+A364G+S388K+D470T+R483V;

V45L+A117S+L149R+S214Q+V258P+A276S+Q314A+H358D+A364G+S388K+D470T+R483V;

V45L+A79C+L149R+S214Q+V258P+A276S+A364G+S388K;

V45L+A117S+L149R+S214Q+V258P+A276S+N355G+H358D+A364G+S388K+D470T+L530R;

V45L+A117S+L149R+S214Q+V258P+A276S+V350L+H358D+A364G+S388K+D470T+L530R;

V45L+A117S+L149R+S214Q+V258P+A276S+H358D+A364G+S388K+D470T+S481S+L530R;

V45L+A117S+L149R+S214Q+V258P+A276S+H358D+A364G+S388K+D470T+Q496E+L530R;

V45L+A117S+L149R+N168G+S214Q+V258P+A276S+H358D+A364G+S388K+D470T+L530R;

V45L+A117S+L149R+S214Q+V258P+A276S+H358D+A364G+S388K+D470T+V498I+L530R;

V45L+A117S+L149R+S214Q+V258P+A276S+H358D+A364G+S388K+D470A+R483V;

V45N+A117S+L149R+S214Q+V258P+A276S+H358D+A364G+S388K+D470T+R483V;

V45L+A117S+L149R+S214Q+V258P+A276S+H358D+A364G+S388K+D470T+R483V+A499Y;

V45L+A79C+L149R+V258P+A276S+Q314A+A364G+S388K;

V45L+A79C+L149R+V258P+A276S+L281V+A364G+S388K;

V45L+A117S+L149R+S214Q+V258P+A276S+A286S+H358D+A364G+S388K+D470T+L530R;

V45L+A117S+L149R+S214Q+V258P+A276S+H358D+A364G+S388K+D470T+S501N+L530R;

V45L+A117S+L149R+S214Q+V258P+A276S+H358D+A364G+S388K+D470T+N476N+L530R;

V45L+A117S+L149R+S214Q+V258P+A276S+H358D+A364G+S388K+D470T+T477V+L530R;

V45L+A117S+L149R+S214Q+V258P+A276S+H358D+A364G+S388H+D470T+L530R;

V45L+A117S+L149R+S214Q+V258P+A276S+H358D+A364G+S388K+D470T+L478N+L530R;

V45L+S92D+A117S+L149R+S214Q+V258P+A276S+H358D+A364G+S388K+D470T+L530R;

V45L+A117S+L149R+S214Q+V258P+A276S+H358D+A364G+S388K+D470T+V500V+L530R;

V45L+A117S+L149R+S214Q+V258P+A276S+H358D+A364G+S388K+D470T+R483V+V500V;

V45L+A117S+L149R+S214Q+V258P+A276S+H358D+A364G+S388K+D470T+L478P+L530R;

V45L+A117S+L149R+S214Q+V258P+A276S+H358D+A364G+S388K+D470T+R483V+S501E;

V45L+A117S+L149R+S214Q+V258P+A276S+H358D+A364G+S388K+D470T+D525L+L530R;

V45L+A117S+L149R+S214Q+V258P+A276S+H358D+A364G+S388K+D470T+G526T+L530R;

V45L+A117S+L149R+S214Q+V258P+A276S+H358D+A364G+S388K+D470T+N527H+L530R;

V45L+A117S+L149R+S214Q+V258P+A276S+H358D+A364G+S388K+D470T+T477K+L530R; M28P+V45L+A117S+L149R+S214Q+V258P+A276S+H358D+A364G+S388K+D470T+L530R; D9E+V45L+A117S+L149R+S214Q+V258P+A276S+H358D+A364G+S388K+D470T+R483V;

V45L+A117S+L149R+S214Q+V258P+A276S+H358D+A364G+S388K+D470T+S501E+L530R;

V45L+A117S+L149R+S214Q+W250Y+V258P+A276S+H358D+A364G+S388K+D470T+L530R;

V45L+A117S+L149R+S214Q+V258P+A276S+H358D+A364G+S388K+D470T+A499L+L530R;

V45L+A117S+L149R+S214Q+V258P+A276S+H358D+A364G+S388K+D470T+A472I+L530R;

V45L+A117S+N127D+L149R+S214Q+V258P+A276S+H358D+A364G+S388K+D470T+R483V;

V45L+A117S+L149R+S214Q+V258P+A276S+H358D+A364G+S388K+D470T+V500T+L530R;

V45L+A117S+L149R+S214Q+V258P+A276S+H358D+A364G+S388K+D470T+R483V+N527H;

V45N+A117S+L149R+S214Q+V258P+A276S+H358D+A364G+S388K+D470T+L530R;

V45L+A117S+L149R+S214Q+V258P+A276S+H358D+A364G+S388K+D470T+T480N+L530R;

V45L+A117S+L149R+S214Q+V258P+A276S+H358D+A364G+S388K+D470T+R483V+V497P;

V45L+A117S+L149R+S214Q+V258P+A276S+H358D+A364G+S388K+D470T+R483V+V498I;

V45L+A79C+L149R+S173Y+V258P+A276S+A364G+S388K;

V45L+A117S+L149R+S214Q+V258P+A276S+H358D+A364G+S388K+D470T+T480N+R483V;

V45L+A117S+L149R+S214Q+H219V+V258P+A276S+H358D+A364G+S388K+D470T+L530R;

V45L+A117S+L149R+S214Q+V258P+A276S+H358D+A364G+S388A+D470T+L530R;

M28P+V45L+A117S+L149R+S214Q+V258P+A276S+H358D+A364G+S388K+D470T+R483V;

V45L+A117S+L149R+A170S+S214Q+V258P+A276S+H358D+A364G+S388K+D470T+R483V;

V45L+A79C+L149R+G187K+V258P+A276S+A364G+S388K;

V45L+A117S+L149R+S214Q+V258P+A276S+H358D+A364G+S388K+S389H+D470T+L530R;

V45L+D113T+A117S+L149R+S214Q+V258P+A276S+H358D+A364G+S388K+D470T+L530R;

V45L+A117S+L149R+N163H+S214Q+V258P+A276S+H358D+A364G+S388K+D470T+R483V;

V45L+A117S+L149R+S214Q+V258P+A276S+H358D+A364G+S388A+D470T+R483V;

V45L+A117S+L149R+S214Q+V258P+A276S+H358D+A364G+S388L+D470T+R483V;

V45L+Q61R+A79C+L149R+V258P+A276S+A364G+S388K;

V45L+A79C+L149R+N168G+V258P+A276S+A364G+S388K;

V45L+A117S+L149R+T211H+S214Q+V258P+A276S+H358D+A364G+S388K+D470T+L530R;

V45L+A79C+L149R+T215V+V258P+A276S+A364G+S388K;

V45L+A117S+L149R+S214Q+V258P+A276S+H358D+A364G+S388K+D470T+R483V+V500T;

V45L+A79C+L149R+V258P+A276S+A286S+A364G+S388K;

V45L+A79C+L149R+H219V+V258P+A276S+A364G+S388K;

V45L+A79C+L149R+A170S+V258P+A276S+A364G+S388K;

V45L+D113T+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A79C+L149R+V258P+A276S+A364G+S388K;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+T215I+V258P+A276S+H358D+A364G+S388K+D470T+R483V;

V45L+A117S+L149R+S214Q+H219I+V258P+A276S+H358D+A364G+S388K+D470T+R483V;

V45L+A117S+L149R+S214Q+V258P+A276S+H358D+A364G+S388K+D470T+L478N;

V45L+A117S+L149R+S214Q+V258P+A276S+L281V+H358D+A364G+S388K+D470T;

V45L+A117S+L149R+S214E+V258P+A276S+H358D+A364G+S388K+D470T;

V45L+T70R+A117S+L149R+S214Q+V258P+A276S+L281V+H358D+A364G+S388K+G394A+C468L+D470T+R483V;

V45L+A117S+L149R+S214Q+V258P+A276S+H358D+A364G+Q385C+S388K+D470T+L530R;

V45L+A117S+L149R+S214Q+V258P+A276S+H358D+A364G+T366I+S388K+D470T;

V45L+A117S+L149R+S214Q+V258P+A276S+H358D+A364G+S388K+D470T+S473G;

V45L+A117S+L149R+S214Q+V258P+A276S+H358D+A364G+S388K+D470T+L478P;

V45L+A117S+L149R+S214Q+V258P+A276S+H358D+A364G+S388K+G394D+D470T;

V45L+A117S+L149R+S214Q+V258P+A276S+A364G+Q384A+S388K+D470T+R483V; D11E+V45L+A79C+L149R+V258P+A276S+A364G+S388K;

V45L+A117S+L149R+S214Q+V258P+A276S+H358D+A364G+S388K+D470T+R483V+L530R;

V45L+A117S+L149R+S214Q+V258P+A276S+H358D+A364G+S388K+D470T+N529T;

V45L+A117S+L149R+S214Q+V258P+A276S+H358D+A364G+S388K+G394Q+D470T;

V45L+A117S+L149R+G187L+S214Q+V258P+A276S+H358D+A364G+S388K+D470T;

V45L+A79C+L149R+V258P+A276S+A286E+A364G+S388K;

V45L+A117S+L149R+S214Q+V258P+A276S+H358D+A364G+S388K+D470T+R483V+G503L;

V45L+A117S+L149R+S214Q+V258P+A276S+Q314A+H358D+A364G+S388K+D470T;

V45L+A117S+L149R+S214Q+V258P+A276S+V350L+H358D+A364G+S388K+D470T;

V45L+A117S+L149R+G187K+S214Q+V258P+A276S+H358D+A364G+S388K+D470T;

V45L+A117S+L149R+S214Q+W250Y+V258P+A276S+H358D+A364G+S388K+D470T;

V45L+A117S+L149R+S214Q+V258P+A276S+A286E+H358D+A364G+S388K+D470T;

V45L+A117S+L149R+S214Q+V258P+A276S+H358D+A364G+Q384A+S388K+D470T;

V45L+A117S+L149R+S214Q+V258P+A276S+H358D+A364G+S388R+D470T;

V45L+A117S+L149R+S214Q+V258P+A276S+H358D+A364G+S388K+D470T+S528H V45L+A117S+L149R+S214Q+T215V+V258P+A276S+H358D+A364G+S388K+D470T+R483V;

V45L+A117S+L149R+S214Q+V258P+A276S+H358D+A364G+S388K+D470T+Q496R;

V45L+A117S+L149R+N163H+S214Q+V258P+A276S+H358D+A364G+S388K+D470T;

V45L+A117S+L149R+S214Q+V258P+A276S+S282N+H358D+A364G+S388K+D470T;

V45L+A117S+L149R+S214Q+V258P+A276Q+H358D+A364G+S388K+D470T; D11E+V45L+A117S+L149R+S214Q+V258P+A276S+H358D+A364G+S388K+D470T;

V45L+A117S+L149R+S214Q+V258P+A276S+H358D+A364G+T366V+S388K+D470T;

V45L+A117S+L149R+S214Q+V258P+A276S+H358D+A364G+S388K+D470T+S473K;

V45L+A79C+L149R+T180Q+V258P+A276S+A364G+S388K;

V45L+A117S+L149R+S214Q+V258P+A276S+H358D+A364G+S388K+D470T+S481A+R483V;

V45L+A117S+L149R+S214Q+V258P+A276S+H358D+A364G+S388R+D470T+R483V;

V45L+A117S+L149R+S214Q+V258P+A276S+Q314A+A364G+S388K+D470T+R483V;

V45L+S92D+A117S+L149R+S214Q+V258P+A276S+H358D+A364G+S388K+D470T;

V45L+A117S+L149R+S214Q+V258P+N260H+A276S+H358D+A364G+S388K+D470T;

V45L+A117S+L149R+G187S+S214Q+V258P+A276S+H358D+A364G+S388K+D470T;

V45L+A117S+L149R+S214Q+V258P+A276S+H358D+A364G+S388K+D470T+T477K;

V45L+A117S+L149R+S214Q+V258P+A276S+H358D+A364G+S388K+D470T+N474G;

V45L+A117S+L149R+S214Q+V258P+A276S+H358D+A364G+S388K+N395D+D470T;

V45L+A117S+L149R+S214Q+V258P+A276S+H358D+A364G+S388K+D470T+S501N;

V45L+A117S+N127D+L149R+S214Q+V258P+A276S+H358D+A364G+S388K+D470T;

V45L+A117S+L149R+S214Q+V258P+A276S+H358D+A364G+S388K+V393R+D470T;

V45L+A117S+L149R+S173Y+S214Q+V258P+A276S+H358D+A364G+S388K+D470T;

V45L+A117S+L149R+S214Q+V258P+A276S+H358D+A364G+S388H+D470T;

V45L+A117S+L149R+S214Q+T215V+V258P+A276S+H358D+A364G+S388K+D470T+L530R;

V45L+A117S+L149R+G187S+S214Q+V258P+A276S+H358D+A364G+S388K+D470T+L530R;

V45L+A117S+L149R+T211H+S214Q+V258P+A276S+H358D+A364G+S388K+D470T+R483V;

V45L+A117S+L149R+S214Q+V258P+A276S+H358D+A364G+S388K+D470T+T480N;

V45L+A117S+L149R+N168G+S214Q+V258P+A276S+H358D+A364G+S388K+D470T;

V45L+A79C+L149R+H219I+V258P+A276S+A364G+S388K;

V45L+L149R+S214E+V258P+A276S+H358D+A364G+D470T;

V45L+Q61K+A117S+L149R+S214Q+V258P+A276S+A364G+S388K+D470T+R483V;

V45L+D113T+A117S+L149R+S214Q+V258P+A276S+H358D+A364G+S388K+D470T;

V45L+A117S+L149R+S214Q+V258P+A276S+A364G+T366V+S388K+D470T+R483V;

V45L+A117S+L149R+S214Q+V258P+A276S+H358T+A364G+S388K+D470T+R483V;

V45L+A117S+L149R+S214Q+V258P+A276S+A364G+S388K+V393R+D470T+R483V;

V45N+A79C+L149R+V258P+A276S+A364G+S388K;

V45L+A117S+L149R+S214Q+V258P+A276S+H358D+A364G+S388K+D470T+Q496Q;

V45L+A117S+L149R+S214Q+V258P+A276S+A364G+S388K+G394Q+D470T+R483V;

V45L+A79C+L149R+V258P+A276S+V350L+A364G+S388K;

V45L+L149R+S214E+V258P+A276S+H358D+A364G+D470T;

V45L+A117S+L149R+S214Q+V258P+A276S+A364G+T366I+S388K+D470T+R483V;

V45L+A117S+L149R+S214Q+V258P+A276S+H358D+A364G+S388K+D470T+R483V+G526T;

V45L+A117S+L149R+S214Q+V258P+A276S+H358D+A364G+S388K+D470T+L484S;

V45L+A79C+A117S+L149R+S214Q+V258P+A276S+H358D+A364G+S388K;

V45L+A117S+L149R+S214Q+V258P+A276S+H358D+A364G+S388K+D470T+A499L;

V45L+A117S+L149R+S214Q+V258P+A276S+H358T+A364G+S388K+D470T+L530R;

V45L+A79C+D113T+L149R+V258P+A276S+A364G+S388K;

V45L+A117S+L149R+S214Q+V258P+A276S+H358D+A364G+S388K+D470T+R483V+D525L;

V45L+A117S+L149R+S214Q+V258P+A276S+H358D+A364G+S388K+D470T+V498I;

V45L+A117S+L149R+S214Q+V258P+A276S+H358D+A364G+S388L+D470T;

V45L+T70R+A117S+L149R+S214Q+V258P+A276S+A364G+S388K+D470T+R483V;

V45L+A117S+L149R+S214Q+V258P+A276S+H358D+A364G+S388K+D470T+S528H+L530R;

V45L+A117S+L149R+S214Q+V258P+A276S+A364G+S388K+D470T+N476D+R483V; D9E+V45L+A117S+L149R+S214Q+V258P+A276S+A364G+S388K+D470T+S501E;

V45L+A117S+L149R+S214Q+V258P+A276S+H358D+A364G+S388K+D470T+S481A;

V45L+A117S+L149R+S214Q+H219V+V258P+A276S+H358D+A364G+S388K+D470T;

V45L+A117S+L149R+S214Q+V258P+A276S+H358D+A364G+S388K+D470T+S481S;

V45L+A117S+L149R+G187K+S214Q+V258P+A276S+A364G+S388K+D470T+R483V;

V45L+A117S+L149R+S214Q+V258P+A276S+A364G+S388K+N395D+D470T+R483V;

V45L+A117S+L149R+S214Q+V258P+A276S+H358D+A364G+S388K+I469T+D470T+R483V;

V45L+A79C+N127D+L149R+V258P+A276S+A364G+S388K; D9E+V45L+A79C+L149R+V258P+A276S+A364G+S388K;

V45L+L149R+S214E+V258P+A276S+H358D+A364G+D470T;

V45L+A117S+L149R+G187L+S214Q+V258P+A276S+A364G+S388K+D470T+R483V;

V45L+A117S+L149R+S214Q+V258P+A276S+H358D+A364G+S388K+D470T+N529T+L530R;

V45L+A79C+L149R+W250Y+V258P+A276S+A364G+S388K;

V45L+A117S+L149R+S214Q+V258P+A276S+H358D+A364G+S388K+D470T+V497P;

V45L+A117S+L149R+S214Q+V258P+A276S+H358D+A364G+S388K+D470T+N527H;

V45L+A117S+L149R+S214Q+V258P+A276S+A364G+S388K+D470T+R483V+Q496Q;

V45L+A117S+L149R+N168G+S214Q+V258P+A276S+A364G+S388K+D470T+R483V;

V45L+A117S+L149R+S214Q+V258P+A276S+A364G+S388K+D470T+N476N+R483V;

V45L+A117S+L149R+S214Q+V258P+A276S+A364G+S388K+D470T+T477V+R483V;

V45L+A117S+L149R+S214Q+V258P+A276S+V350L+A364G+S388K+D470T+R483V;

V45L+A117S+L149R+S214Q+V258P+A276S+A364G+S388K+D470T+L478N+R483V;

V45L+A117S+L149R+S214E+V258P+A276S+A364G+S388K+D470T+R483V; M28P+V45L+A117S+L149R+S214Q+V258P+A276S+H358D+A364G+S388K+D470T;

V45L+A117S+L149R+S214Q+V258P+A276S+A364G+S388K+V393H+D470T+R483V;

V45L+Q61R+A117S+L149R+S214Q+V258P+A276S+A364G+S388K+D470T+R483V;

V45L+A117S+L149R+S214Q+V258P+A276S+A364G+S388K+D470T+R483V+V497P;

V45L+A117S+L149R+S214Q+V258P+A276S+A364G+S388K+D470T+R483V+V498I;

V45L+A117S+L149R+S214Q+V258P+A276S+S282N+H358D+A364G+S388K+D470T+L530R;

V45L+T70R+A117S+L149R+S214Q+V258P+A276S+L281V+H358D+A364G+C375V+S388K+G394A+D470T+R483V;

V45L+A117S+L149R+S214Q+V258P+A276S+A286E+A364G+S388K+D470T+R483V;

V45L+A117S+L149R+S214Q+V258P+A276S+A364G+S388R+D470T+R483V;

V45L+A117S+L149R+S214Q+V258P+A276S+A364G+S388K+D470A+R483V;

V45L+A117S+L149R+S214Q+V258P+A276S+A364G+S388K+D470T+T477K+R483V;

V45L+A117S+L149R+S214Q+V258P+A276S+A364G+S388K+D470T+R483V+Q496E;

V45L+A117S+L149R+S214Q+V258P+A276S+A364G+S388K+D470T+R483V+Q496R;

V45L+A117S+L149R+S214Q+V258P+A276S+A364G+S388K+D470T+R483V+A499L;

V45L+L149R+S214E+V258P+A276S+H358D+A364G+D470T;

V45L+L149R+S214E+V258P+A276S+H358D+A364G+D470T;

V45L+A117S+L149R+S214Q+T215I+V258P+A276S+H358D+A364G+S388K+D470T;

V45L+T70R+A117S+L149R+S214Q+V258P+A276S+L281V+H358D+A364G+S388K+G394A+C468S+D470T+R483V;

V45L+A117S+L149R+S214Q+V258P+A276S+A364G+S388K+Y391V+D470T+R483V:

V45L+A117S+L149R+S214Q+V258P+A276Q+A364G+S388K+D470T+R483V;

V45L+A117S+L149R+S214Q+V258P+A276S+H358D+A364G+S388K+D470T+G503L+L530R;

V45L+A117S+L149R+S214Q+H219I+V258P+A276S+H358D+A364G+S388K+D470T+L530R;

V45L+A117S+L149R+S214Q+V258P+A276S+H358D+A364G+S388K+D470T+N476D;

V45L+A117S+L149R+S214Q+V258P+A276S+H358D+A364G+S388K+V393H+D470T;

V45L+A117S+L149R+S214Q+V258P+A276S+A364G+S388K+D470T+R483V;

V45L+A117S+L149R+S214Q+V258P+A276S+A286S+A364G+S388K+D470T+R483V;

V45L+A117S+L149R+S214Q+V258P+A276S+A364G+S388K+D470T+R483V+S501E;

V45L+A117S+L149R+S214Q+V258P+A276S+A364G+S388K+D470T+R483V+N529T;

V45L+A117S+L149R+S214Q+V258P+A276S+A364G+S388K+D470T+R483V+S501N;

V45L+A117S+L149R+S214Q+V258P+A276S+H358D+A364G+S388K+D470T;

V45L+A117S+L149R+S214Q+V258P+A276S+A364G+S388K+D470T+S481S+R483V;

V45L+A117S+L149R+S214Q+V258P+A276S+A364G+S388K+D470T+L478P+R483V; D11E+V45L+A117S+L149R+S214Q+V258P+A276S+A364G+S388K+D470T+R483V;

V45L+A117S+L149R+S214Q+V258P+A276S+H358D+A364G+S388K+D470T+T477V;

V45L+A117S+L149R+S214Q+V258P+A276S+H358D+A364G+S388K+D470T+V497P+L530R;

V45L+A117S+L149R+T211H+S214Q+V258P+A276S+H358D+A364G+S388K+D470T;

V45L+A117S+L149R+S214Y+V258P+A276S+H358D+A364G+S388K+D470T+R483V;

V45L+A117S+L149R+S214Q+W250Y+V258P+A276S+A364G+S388K+D470T+R483V;

V45L+S92D+A117S+L149R+S214Q+V258P+A276S+A364G+S388K+D470T+R483V;

V45L+A117S+L149R+S214Q+V258P+A276S+A364G+S388K+D470T+R483V+N527H;

V45N+A117S+L149R+S214Q+V258P+A276S+A364G+S388K+D470T+R483V;

V45L+A117S+L149R+S214Q+V258P+A276S+H358D+A364G+Q385C+S388K+D470T;

V45L+A117S+L149R+S173Y+S214Q+V258P+A276S+A364G+S388K+D470T+R483V;

V45L+A117S+L149R+S214Q+V258P+A276S+A364G+S388H+D470T+R483V;

V45L+A117S+L149R+S214Q+V258P+A276S+A364G+S388K+S389H+D470T+R483V;

V45L+A117S+L149R+S214Q+H219V+V258P+A276S+A364G+S388K+D470T+R483V;

V45L+A79C+A117S+L149R+S214Q+V258P+A276S+A364G+S388K+D470T;

V45L+A117S+L149R+S214Q+T215I+V258P+A276S+H358D+A364G+S388K+D470T+L530R;

V45L+A117S+L149R+S214Q+V258P+A276S+H358D+A364G+S388K+D470T+Q496E;

V45L+A117S+L149R+A170S+S214Q+V258P+A276S+H358D+A364G+S388K+D470T;

V45L+A117S+L149R+S214Q+V258P+A276S+H358D+A364G+S388K+S389V+D470T+R483V;

V45L+A117S+L149R+S214Q+V258P+A276S+H358D+A364G+S388K+S389V+D470T+L530R;

V45L+A117S+L149R+S214Q+V258P+A276S+H358D+A364G+S388A+D470T;

V45L+A117S+L149R+S214Q+V258P+A276S+H358D+A364G+S388K+S389V+D470T;

V45L+T70R+A117S+L149R+S214Q+V258P+A276S+H358D+A364G+S388K+D470T;

V45L+A117S+L149R+S214Q+H219I+V258P+A276S+A364G+S388K+D470T+R483V;

V45L+A79C+L149R+N163H+V258P+A276S+A364G+S388K;

V45L+A117S+L149R+S214Q+V258P+A276S+A364G+S388K+D470T+R483V+A499Y;

V45L+A117S+L149R+S214Q+V258P+A276S+L281V+A364G+S388K+D470T+R483V;

V45L+A117S+L149R+S214Q+V258P+A276S+A364G+S388K+D470T+R483V+V500T;

V45L+A117S+L149R+T180Q+S214Q+V258P+A276S+H358D+A364G+S388K+D470T;

V45L+A117S+L149R+S214Q+V258P+A276S+H358D+A364G+S388K+D470T+V500V;

V45L+A117S+L149R+S214Q+V258P+A276S+H358D+A364G+S388K+D470T+G526T;

V45L+C60L+T70R+A117S+L149R+S214Q+V258P+A276S+L281V+H358D+A364G+S388K+G394A+D470T+R483V;

V45L+C69L+T70R+A117S+L149R+S214Q+V258P+A276S+L281V+H358D+A364G+S388K+G394A+D470T+R483V;

V45L+A117S+L149R+S214Q+V258P+A276S+A364G+S388A+D470T+R483V;

V45L+A117S+L149R+S214Q+V258P+A276S+A286S+H358D+A364G+S388K+D470T;

V45L+A117S+L149R+S214Q+V258P+A276S+H358D+A364G+S388K+D470T+R483V+V497D;

V45L+A117S+L149R+S214Q+V258P+A276S+H358D+A364G+S388K+D470A+L530R;

V45L+A117S+L149R+S214Q+V258P+A276S+A364G+S388K+D470T+R483V+D525L;

V45L+A117S+L149R+S214Q+V258P+A276S+H358D+A364G+S388K+D470T+N476N;

V45L+A117S+L149R+S214Q+V258P+A276S+L281V+A364G+S388K+D470T;

V45L+A117S+L149R+S214Q+V258P+A276S+A364G+Q384A+S388K+D470T;

V45L+A117S+L149R+S214Q+V258P+A276S+A286E+A364G+S388K+D470T;

V45L+A117S+L149R+S214Q+V258P+A276S+A364G+S388K+D470T+T477K;

V45L+A117S+L149R+S214Q+V258P+A276S+H358D+A364G+S388K+S389H+D470T;

V45L+A117S+L149R+S214Y+V258P+A276S+H358D+A364G+S388K+D470T+L530R;

V45L+A117S+L149R+S214Y+V258P+A276S+H358D+A364G+S388K+D470T;

V45L+A117S+L149R+S214Q+V258P+A276S+A364G+S388K+G394A+D470T;

V45L+A117S+L149R+S214Q+V258P+A276S+A364G+S388K+D470T+N476D;

V45L+A117S+L149R+S214Q+V258P+A276S+H358D+A364G+S388K+D470T+L478N+R483V;

V45L+C60A+T70R+A117S+L149R+S214Q+V258P+A276S+L281V+H358D+A364G+S388K+G394A+D470T+R483V;

V45L+A117S+L149R+S214Q+V258P+A276S+A364G+S388K+V393H+D470T;

V45L+A117S+L149R+S214Q+V258P+A276S+A364G+S388K+N395D+D470T; A25W+V45L+A117S+L149R+S214Q+V258P+A276S+H358D+A364G+S388K+D470T;

V45L+T70R+A117S+L149R+S214Q+C243L+V258P+A276S+L281V+H358D+A364G+S388K+G394A+D470T+R483V;

V45L+A117S+L149R+S214Q+V258P+A276S+H358T+A364G+S388K+D470T;

V45L+A117S+L149R+S214Q+V258P+A276S+A364G+S388K+D470T+L478N;

V45L+C60S+T70R+A117S+L149R+S214Q+V258P+A276S+L281V+H358D+A364G+S388K+G394A+D470T+R483V;

V45L+A117S+L149R+S214Q+W250Y+V258P+A276S+A364G+S388K+D470T;

V45L+A117S+L149R+S214Q+T215V+V258P+A276S+A364G+S388K+D470T+R483V;

V45L+Q61R+A117S+L149R+S214Q+V258P+A276S+H358D+A364G+S388K+D470T;

V45L+A117S+L149R+G187L+S214Q+V258P+A276S+A364G+S388K+D470T; D9E+V45L+A117S+L149R+S214Q+V258P+A276S+A364G+S388K+D470T;

V45L+A117S+L149R+S214Q+V258P+A276S+H358D+A364G;

V45L+C69A+T70R+A117S+L149R+S214Q+V258P+A276S+L281V+H358D+A364G+S388K+G394A+D470T+R483V;

V45L+T70R+A117S+L149R+C161S+S214Q+V258P+A276S+L281V+H358D+A364G+S388K+G394A+D470T+R483V;

V45L+A117S+L149R+S214Q+H219I+V258P+A276S+H358D+A364G+S388K+D470T;

V45L+A117S+L149R+S214Q+V258P+A276S+S282N+A364G+S388K+D470T;

V45L+A117S+L149R+T211H+S214Q+V258P+A276S+A364G+S388K+D470T+R483V;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K; D11E+V45L+A117S+L149R+S214Q+V258P+A276S+A364G+S388K+D470T;

V45L+A117S+L149R+S214Q+V258P+A276S+A364G+S388K+D470T+N476N;

V45L+A117S+L149R+S214Q+V258P+A276S+A364G+S388K+G394D+D470T;

V45L+Q61K+A117S+L149R+S214Q+V258P+A276S+A364G+S388K+D470T;

V45L+A117S+L149R+S214Q+V258P+A276S+H358D+A364G+S388K+D470T+G503L;

V45L+A117S+L149R+S214Q+V258P+A276S+A286S+A364G+S388K+D470T;

V45L+A117S+L149R+S214Q+V258P+A276S+H358D+A364G+S388K+D470A;

V45L+A117S+L149R+S214Q+V258P+A276S+A364G+S388K+G394Q+D470T;

V45L+A117S+L149R+S214Q+V258P+A276S+A364G+S388R+D470T;

V45L+A117S+L149R+S214Q+V258P+A276S+A364G+S388K+D470T+L478P;

V45L+A117S+L149R+S214Q+V258P+A276S+V350L+A364G+S388K+D470T;

V45L+D113T+A117S+L149R+S214Q+V258P+A276S+A364G+S388K+D470T;

V45L+A117S+L149R+T180Q+S214Q+V258P+A276S+A364G+S388K+D470T;

V45L+C69V+T70R+A117S+L149R+S214Q+V258P+A276S+L281V+H358D+A364G+S388K+G394A+D470T+R483V;

V45L+A117S+L149R+S214Q+V258P+A276S+H358D+A364G+S388K+D470T+D525L;

V45L+A117S+L149R+S214Q+V258P+A276S+A364G+S388K+D470T;

V45L+A117S+L149R+S214Q+V258P+A276S+H358D+A364G+S388K+D470T+V500T;

V45L+A117S+L149R+S214Q+V258P+A276S+A364G+S388K+S389H+D470T;

V45L+A117S+L149R+S214Q+V258P+A276S+A364G+S388K+V393R+D470T;

V45L+A117S+L149R+S214Q+V258P+A276S+A364G+S388K+D470T+Q496R;

V45L+A117S+N127D+L149R+S214Q+V258P+A276S+A364G+S388K+D470T;

V45L+A117S+L149R+S214Q+V258P+A276S+A364G+S388K+D470T+A499Y;

V45L+A117S+L149R+S214Q+T215V+V258P+A276S+H358D+A364G+S388K+D470T;

V45L+A117S+L149R+S214Q+V258P+A276S+H358D+A364G+S388K+D470T+T480L;

V45L+A117S+L149R+S214Q+V258P+A276S+A364G+S388L+D470T;

V45L+A117S+L149R+S173Y+S214Q+V258P+A276S+A364G+S388K+D470T;

V45L+S92D+A117S+L149R+S214Q+V258P+A276S+A364G+S388K+D470T;

V45L+A117S+L149R+S214Q+V258P+A276S+A364G+S388K+S389V+D470T+R483V;

V45L+A117S+L149R+S214Q+V258P+A276S+A364G+S388K+D470T+N529T;

V45L+C60V+T70R+A117S+L149R+S214Q+V258P+A276S+L281V+H358D+A364G+S388K+G394A+D470T+R483V;

V45L+A117S+L149R+S214Q+V258P+A276S+A364G+S388K+D470T+R483V+V497E;

V45L+A117S+L149R+S214Q+V258P+A276S+H358D+A364G+S388K+L478N;

V45L+T70R+A117S+L149R+S214Q+V258P+A276S+A364G+S388K+D470T; M28P+V45L+A117S+L149R+S214Q+V258P+A276S+A364G+S388K+D470T;

V45L+A117S+L149R+S214Q+V258P+A276Q+A364G+S388K+D470T;

V45L+A117S+L149R+S214Q+V258P+A276S+H358D+A364G+S388K+D470V+R483V;

V45L+A117S+L149R+S214Y+V258P+A276S+A364G+S388K+D470T+R483V;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

V45L+Q61R+A117S+L149R+S214Q+V258P+A276S+A364G+S388K+D470T;

V45L+A117S+L149R+S214Q+T215I+V258P+A276S+A364G+S388K+D470T;

V45L+A117S+L149R+S214Q+V258P+A276S+A364G+S388K+D470T+Q496E;

V45L+A117S+L149R+G187K+S214Q+V258P+A276S+A364G+S388K+D470T;

V45L+A117S+L149R+S214Q+V258P+A276S+A364G+S388K+D470T+W495I;

V45L+A117S+L149R+S214Q+V258P+A276S+A364G+S388K+D470T+V498I;

V45L+A117S+L149R+S214Q+V258P+A276S+H358D+A364G+S388K+D470K+R483V;

V45L+A117S+L149R+S214Q+V258P+A276S+A364G+S388K+D470T+V500V; D11E+V45L+A117S+L149R+S214Q+V258P+A276S+H358D+A364G+S388K;

V45L+A117S+L149R+A170S+S214Q+V258P+A276S+A364G+S388K+D470T;

V45L+A117S+L149R+S214Q+V258P+A276S+A364G+S388K+Y391V+D470T;

V45L+A117S+L149R+S214Q+V258P+A276S+Q314A+A364G+S388K+D470T;

V45L+A117S+L149R+S214Q+V258P+A276S+A364G+S388K+D470T+T477V; A25W+V45L+A79C+L149R+S214E+V258P+A276S+A364G;

V45L+A117S+L149R+S214Q+V258P+A276S+H358D+A364G+S388K+G394Q;

V45L+A117S+L149R+S214Q+H219V+V258P+A276S+A364G+S388K+D470T;

V45L+A117S+L149R+S214Q+V258P+A276S+A364G+S388K+D470T+Q496Q;

V45L+T70R+A117S+L149R+S214Q+V258P+A276S+L281V+H358D+A364G+C375A+S388K+G394A+D470T+R483V;

V45L+A117S+L149R+D192E+S214Q+V258P+A276S+H358D+A364G;

V45L+A117S+L149R+S214Q+V258P+A276S+H358D+A364G+S388K+D470T+A472L;

V45L+A117S+L149R+S214Q+V258P+A276S+H358D+A364G+S388K+A499Y;

V45L+A117S+L149R+S214Q+T215V+V258P+A276S+A364G+S388K+D470T;

V45L+A117S+L149R+S214Q+V258P+A276S+A364G+Q385C+S388K+D470T;

V45L+A117S+L149R+S214Q+V258P+A276S+A364G+S388A+D470T;

V45L+A117S+L149R+S214Q+V258P+A276S+A364G+S388H+D470T;

V45L+A117S+L149R+S214Q+V258P+A276S+A364G+S388K+D470T+T480N;

V45L+A117S+L149R+S214Q+V258P+A276S+A364G+S388K+D470T+V497P;

V45L+A117S+L149R+S214Q+V258P+A276S+A364G+S388K+D470T+A499L;

V45L+A117S+L149R+S214Q+V258P+A276S+A364G+S388K+D470T+S501N;

V45L+A117S+L149R+S214Q+V258P+A276S+A364G+D470T;

V45L+A117S+L149R+S214Q+V258P+A276S+H358D+A364G+Q384A+S388K;

V45L+T70R+A117S+L149R+S214Q+V258P+A276S+L281V+H358D+A364G+C375S+S388K+G394A+D470T+R483V;

V45L+A117S+L149R+S214Q+V258P+A276S+A364G+S388L+D470T+R483V;

V45L+A79C+S92D+L149R+V258P+A276S+A364G+S388K;

V45L+T70R+A117S+L149R+S214Q+V258P+A276S+L281V+H358D+A364G+S388K+G394A+D470T+R483V+C540A;

V45N+A117S+L149R+S214Q+V258P+A276S+A364G+S388K+D470T;

V45L+A117S+L149R+S214Q+V258P+A276S+H358D+A364G+S388K+G394A;

V45L+C69S+T70R+A117S+L149R+S214Q+V258P+A276S+L281V+H358D+A364G+S388K+G394A+D470T+R483V;

V45L+A117S+L149R+S214Q+V258P+A276S+H358D+A364G+S388K+V498I;

V45L+A117S+L149R+S214Q+V258P+A276S+H358D+A364G;

V45L+A117S+L149R+S214Q+H219I+V258P+A276S+A364G+S388K+D470T;

V45L+A117S+L149R+N163H+S214Q+V258P+A276S+A364G+S388K+D470T;

V45L+L149R+S214Q+V258P+A276S+H358D+A364G;

V45L+A117S+L149R+S214Q+V258P+A276S+Q314A+H358D+A364G+S388K;

V45L+A117S+L149R+S173Y+S214Q+V258P+A276S+H358D+A364G+S388K;

V45L+A117S+L149R+S214E+V258P+A276S+H358D+A364G+S388K;

V45L+A117S+L149R+S214Q+V258P+A276S+H358D+A364G+S388K+G394D; A25W+V45L+A79C+L149R+V258P+A276S+A364G;

V45L+L149R+S214Q+V258P+A276S+A286E+A364G+S388K;

V45L+Q61R+A117S+L149R+S214Q+V258P+A276S+H358D+A364G+S388K;

V45L+A117S+L149R+S214Q+V258P+A276S+H358D+A364G+T366V+S388K;

V45L+A117S+L149R+D192E+S214Q+V258P+A276S+H358D+A364G;

V45L+A117S+L149R+T180Q+S214Q+V258P+A276S+H358D+A364G+S388K;

V45L+A117S+L149R+S214Q+T215I+V258P+A276S+H358D+A364G+S388K;

V45L+A117S+L149R+S214Q+W250Y+V258P+A276S+H358D+A364G+S388K;

V45L+L149R+S214Q+T215I+V258P+A276S+A364G+S388K;

V45L+A117S+L149R+S214Q+V258P+A276S+H358D+A364G+S388K+N476D;

V45L+D113T+A117S+L149R+S214Q+V258P+A276S+A364G+S388K+D470T+R483V;

V45L+A117S+L149R+S214Q+T215K+V258P+A276S+A364G+S388K+D470T+R483V;

V45L+A117S+L149R+S214Q+V258P+A276S+H358D+A364G+S388K;

V45L+A117S+L149R+S214Q+V258P+A276S+H358D+A364G+S388K+T477V;

V45L+A117S+L149R+S214Q+V258P+A276S+H358D+A364G+S388K+L478P;

V45L+A117S+L149R+S214Q+V258P+A276S+H358D+A364G+S388K+S481A; A25W+V45L+L149R+S214Q+V258P+A276S+A364G+S388K;

V45L+A117S+L149R+T211H+S214Q+V258P+A276S+A364G+S388K+D470T;

V45L+A117S+L149R+S214Q+T215K+V258P+A276S+H358D+A364G+S388K+D470T+R483V;

V45L+A79C+L149R+V258P+A276S+L281V+A364G;

V45L+A117S+L149R+S214Q+V258P+A276S+A364G+S388K+R483V;

V45L+A117S+L149R+S214Q+V258P+A276S+H358D+A364G+S388K+V500T;

V45L+A117S+L149R+S214Q+V258P+A276S+V350L+H358D+A364G+S388K;

V45L+A117S+L149R+G187K+S214Q+V258P+A276S+H358D+A364G+S388K;

V45L+T70R+A117S+L149R+S214Q+V258P+A276S+L281V+H358D+A364G+C375L+S388K+G394A+D470T+R483V;

V45L+L149R+A170S+S214Q+V258P+A276S+A364G+S388K;

V45L+A117S+L149R+S214E+V258P+A276S+A364G+S388K+D470T;

V45L+A117S+L149R+S214Q+V258P+A276S+A364G+S388K+D470V+R483V;

V45L+A117S+L149R+S214Q+V258P+A276S+H358D+A364G+S388K+V500V;

V45L+Q61K+L149R+S214Q+V258P+A276S+A364G+S388K;

V45L+L149R+N168G+S214Q+V258P+A276S+A364G+S388K;

V45L+A117S+L149R+N163H+S214Q+V258P+A276S+H358D+A364G+S388K;

V45L+A79C+D95D+L149R+V258P+A276S+A364G+S388K;

V45L+Q77E+A79C+L149R+V258P+A276S+A364G+S388K;

V45L+A117S+L149R+S214Q+V258P+A276S+A364G+S388K+D470T+R483V+V497W;

V45L+L149R+S214Q+V258P+A276S+S282N+A364G+S388K;

V45L+A117S+L149R+S214Q+V258P+A276S+H358D+A364G+S388R;

V45L+A117S+L149R+S214Q+V258P+A276S+H358D+A364G+S388K+S481S;

V45L+A117S+L149R+S214Q+V258P+A276S+A364G+S388K+D470T+V500T;

V45L+T70R+A117S+L149R+S214Q+V258P+A276S+L281V+H358D+A364G+S388K+G394A+D470T+R483V+C540L;

V45L+T70R+A117S+L149R+S214Q+V258P+A276S+L281V+H358D+A364G+S388K+G394A+D470T+R483V+C522S;

V45L+A117S+L149R+S214Q+V258P+A276S+H358D+A364G+S388K+W495S;

V45L+L149R+S214Q+V258P+A276S+Q314A+A364G+S388K;

V45L+D113T+A117S+L149R+S214Q+V258P+A276S+H358D+A364G+S388K;

V45L+A117S+L149R+S214Q+V258P+A276S+H358D+A364G+S388K+D470T+R483V+V497W;

V45L+T70R+A117S+L149R+S214Q+V258P+A276S+L281V+H358D+A364G+S388K+G394A+D470T+R483V+C540S;

V45L+T70R+A117S+L149R+S214Q+V258P+A276S+L281V+H358D+A364G+S388K+G394A+C396S+D470T+R483V;

V45L+T70R+L149R+S214Q+V258P+A276S+A364G+S388K;

V45L+A117S+L149R+S214Q+V258P+A276S+H358D+A364G+S388K+N474G;

V45L+A117S+L149R+S214Q+V258P+A276S+S282N+H358D+A364G+S388K;

V45L+A117S+L149R+S214Q+V258P+A276S+H358D+A364G+S388K+Q496Q;

V45L+A117S+L149R+S214Q+V258P+A276S+A364G+S388K+S389V+D470T;

V45L+T70R+A117S+L149R+S214Q+V258P+A276S+L281V+H358D+A364G+S388K+G394A+C396A+D470T+R483V;

V45L+A117S+L149R+S214Q+V258P+A276S+H358D+A364G+S388K+S501E; A25W+V45L+L149R+S214E+V258P+A276S+A364G;

V45L+L149R+S214Q+W250Y+V258P+A276S+A364G+S388K;

V45L+L149R+G187K+S214Q+V258P+A276S+A364G+S388K;

V45L+A117S+L149R+S214Q+V258P+A276S+H358D+A364G+S388K+N476N;

V45L+A117S+L149R+S214Q+V258P+A276S+H358D+A364G+S388K+S473K;

V45L+A117S+L149R+G187L+S214Q+V258P+A276S+H358D+A364G+S388K; C40L+V45L+T70R+A117S+L149R+S214Q+V258P+A276S+L281V+H358D+A364G+S388K+G394A+D470T+R483V;

V45L+A117S+L149R+S214Q+V258P+A276S+H358D+A364G+S388K+Q496E; A25W+V45L+A117S+L149R+S214Q+V258P+A276S+H358D+A364G+S388K;

V45L+L149R+V258P+A276S+L281V+H358T+A364G+S388K;

V45L+A117S+L149R+S214Q+V258P+A276S+H358D+A364G+S388A;

V45L+A117S+L149R+N168G+S214Q+V258P+A276S+H358D+A364G+S388K;

V45L+A117S+L149R+S214Q+V258P+A276S+H358D+A364G+S388K+S389H; M28P+V45L+L149R+S214Q+V258P+A276S+A364G+S388K;

V45L+A117S+L149R+S214Q+V258P+A276S+A364G+S388K+D470T+T480L;

V45L+A117S+L149R+S214Q+V258P+A276S+A364G+Q384A+S388K;

V45L+A93S+L149R+A252S+H334D; C40A+V45L+T70R+A117S+L149R+S214Q+V258P+A276S+L281V+H358D+A364G+S388K+G394A+D470T+R483V;

V45L+L149R+G187L+S214Q+V258P+A276S+A364G+S388K;

V45L+A117S+L149R+S214Q+V258P+A276S+H358D+A364G+Q385C+S388K;

V45L+L149R+S214Q+V258P+A276S+A286S+A364G+S388K;

V45L+S92D+A117S+L149R+S214Q+V258P+A276S+H358D+A364G+S388K;

V45L+A117S+L149R+S214Q+V258P+A276S+H358D+A364G+S388L;

V45L+A117S+L149R+S214Q+V258P+A276S+A286E+A364G+S388K; D9E+V45L+L149R+S214Q+V258P+A276S+A364G+S388K;

V45L+L149R+S173Y+S214Q+V258P+A276S+A364G+S388K;

V45L+A117S+L149R+A170S+S214Q+V258P+A276S+H358D+A364G+S388K;

V45L+A117S+L149R+S214Q+V258P+A276S+H358D+A364G+S388K+Y391V;

V45L+A117S+L149R+S214Q+V258P+A276S+L281V+A364G+S388K;

V45L+A117S+L149R+S214Q+V258P+A276S+A364G+S388K+G394D;

V45L+A117S+L149R+S214Q+V258P+A276S+A364G+S388K+S473G;

V45L+A79C+L149R+V258P+A276S+H358T+A364G+S388K;

V45L+L149R+S214Q+V258P+A276Q+A364G+S388K; A25W+V45L+L149R+S214E+V258P+A276S+H358T+A364G;

V45L+A117S+L149R+S214Q+V258P+A276S+H358D+A364G+S388K+T480N; D9E+V45L+A117S+L149R+S214Q+V258P+A276S+H358D+A364G+S388K;

V45L+A117S+L149R+S214Q+H219I+V258P+A276S+H358D+A364G+S388K; C40S+V45L+T70R+A117S+L149R+S214Q+V258P+A276S+L281V+H358D+A364G+S388K+G394A+D470T+R483V; C40V+V45L+T70R+A117S+L149R+S214Q+V258P+A276S+L281V+H358D+A364G+S388K+G394A+D470T+R483V; A25W+V45L+L149R+S214E+V258P+A276S+A286E+A364G;

V45L+A117S+L149R+S214Q+V258P+A276S+A364G+S388K+G394A;

V45L+A117S+L149R+S214Q+V258P+A276S+H358D+A364G+S388K+A499L; A25W+V45L+Q61K+L149R+S214E+V258P+A276S+A364G;

V45L+A117S+L149R+S214Q+T215V+V258P+A276S+H358D+A364G+S388K; A25W+V45L+L149R+V258P+A276S+H358T+A364G+S388K;

V45L+A117S+L149R+S214Q+V258P+A276S+A364G+S388K+D470T+R483V+W495E; A25W+V45L+A79C+L149R+V258P+A276S;

V45L+L149R+S214Q+V258P+A276S+V350L+A364G+S388K; D11E+V45L+L149R+S214Q+V258P+A276S+A364G+S388K;

V45L+L149R+H219I+V258P+A276S+H358T+A364G+S388K;

V45L+T70R+A117S+L149R+S214Q+V258P+A276S+L281V+H358D+A364G+S388K+G394A+C396V+D470T+R483V;

V45N+L149R+S214Q+V258P+A276S+A364G+S388K;

V45L+A117S+L149R+S214Q+H219V+V258P+A276S+H358D+A364G+S388K;

V45N+A117S+L149R+S214Q+V258P+A276S+H358D+A364G+S388K;

V45L+S92D+L149R+S214Q+V258P+A276S+A364G+S388K;

V45L+L149R+V258P+A276S+H358T+A364G;

V45L+A117S+L149R+S214Q+V258P+A276S+A364G+S388K+L478P;

V45L+A117S+L149R+S214Q+V258P+A276S+A364G+S388K+S481A;

V45L+A117S+L149R+S214Q+V258P+A276S+Q314A+A364G+S388K; A25W+V45L+L149R+T211H+S214E+V258P+A276S+A364G; A25W+V45L+L149R+S214E+H219V+V258P+A276S+A364G;

V45L+A117S+L149R+S214Q+V258P+A276S+H358D+A364G+S388K+S389V;

V45L+A117S+L149R+S214Q+V258P+A276S+H358D+A364G+S388K+V393H;

V45L+A117S+L149R+S214Q+V258P+A276Q+H358D+A364G+S388K;

V45L+A117S+N127D+L149R+S214Q+V258P+A276S+H358D+A364G+S388K;

V45L+L149R+W250Y+V258P+A276S+H358T+A364G+S388K; D9E+V45L+L149R+V258P+A276S+H358T+A364G+S388K;

V45L+L149R+H219V+V258P+A276S+H358T+A364G+S388K;

V45L+A117S+L149R+S214Q+V258P+A276S+H358D+A364G+S388K+A472L; A25W+V45L+L149R+V258P+A276S+A364G;

V45L+A117S+L149R+G187K+S214Q+V258P+A276S+A364G+S388K;

V45L+A117S+L149R+S214Q+V258P+A276S+A364G+S388K+L478N;

V45L+A117S+L149R+S214Q+V258P+A276S+A364G+S388K+V393R;

V45L+A117S+L149R+S214E+V258P+A276S+A364G+S388K;

V45L+A117S+L149R+S214Q+V258P+A276S+A364G+T366V+S388K;

V45L+A117S+L149R+S214Q+V258P+A276S+H358T+A364G+S388K; D11E+A25W+V45L+L149R+S214E+V258P+A276S+A364G;

V45L+T70R+A117S+L149R+C161V+S214Q+V258P+A276S+L281V+H358D+A364G+S388K+G394A+D470T+R483V;

V45L+A117S+L149R+T211H+S214Q+V258P+A276S+H358D+A364G+S388K;

V45L+L149R+V258P+A276S+Q314A+H358T+A364G+S388K; D11E+V45L+A117S+L149R+S214Q+V258P+A276S+A364G+S388K;

V45L+A117S+L149R+S214Q+V258P+A276S+A364G+S388K+G394Q;

V45L+A117S+L149R+S214Q+V258P+A276S+A364G+S388K+N395D;

V45L+A117S+L149R+S214Q+V258P+A276S+A364G+S388K+V393H;

V45L+A117S+L149R+S214Q+V258P+A276S+A364G+S388K+S501E;

V45L+A117S+L149R+S214Q+V258P+A276S+A364G+S388K+S528H;

V45L+T70R+A117S+L149R+S214Q+V258P+A276S+H358D+A364G+S388K+D470T+L530R;

V45L+A117S+L149R+S214Q+V258P+A276S+A364G+S388L;

V45L+T70R+A117S+L149R+S214Q+V258P+A276S+L281V+H358D+A364G+S388K+G394A+C396L+D470T+R483V;

V45L+L149R+T180Q+S214Q+V258P+A276S+A364G+S388K;

V45L+D113T+L149R+S214Q+V258P+A276S+A364G+S388K;

V45L+A117S+L149R+S214Q+V258P+A276S+H358D+A364G+S388K+T480L;

V45L+A93S+L149R+A252S+H334D;

V45L+A93S+L149R+V234P+A252S;

V45L+A117S+L149R+S214Q+V258P+A276S+H358D+A364G+S388H; A25W+V45L+Q61R+L149R+S214E+V258P+A276S+A364G;

V45L+A117S+L149R+S214Q+T215K+V258P+A276S+H358D+A364G+S388K+D470T;

V45L+A117S+L149R+S214Q+V258P+A276S+A364G+S388K+S389H;

V45L+A117S+L149R+S214Q+V258P+A276S+A364G+T366I+S388K;

V45L+A117S+L149R+S214Q+V258P+A276S+A364G+S388K+N474G; A25W+V45L+L149R+S214E+V258P+A276S+L281V+A364G; D9E+A25W+V45L+L149R+S214E+V258P+A276S+A364G;

V45L+A117S+L149R+S214Q+T215K+V258P+A276S+H358D+A364G+S388K+D470T+L530R;

V45L+A117S+L149R+S214Y+V258P+A276S+H358D+A364G+S388K;

V45L+A117S+L149R+S214Q+V258P+A276S+H358D+A364G+S388K+V393R;

V45L+Q61R+L149R+V258P+A276S+H358T+A364G+S388K;

V45L+L149R+S214Q+V258P+A276S+A364G+S388K;

V45L+D113T+L149R+V258P+A276S+H358T+A364G+S388K;

V45L+A117S+L149R+S214Q+V258P+A276S+A286S+A364G+S388K;

V45L+A117S+L149R+S214Q+V258P+A276S+A364G+S388R;

V45L+A117S+L149R+S214Q+V258P+A276S+A364G+S388K+A499Y;

V45L+A117S+L149R+S214Q+V258P+A276S+A364G+S388K+V500V;

V45L+Q61R+L149R+S214Q+V258P+A276S+A364G+S388K;

V45L+L149R+S214E+V258P+A276S+H358T+A364G;

V45L+A117S+L149R+S214Q+V258P+A276Q+A364G+S388K;

V45L+L149R+V258P+A276S+L281V+A364G;

V45L+A117S+L149R+S214Q+W250Y+V258P+A276S+A364G+S388K;

V45L+A117S+L149R+S214Q+V258P+A276S+A364G+S388K+T480N;

V45L+A117S+L149R+G187S+S214Q+V258P+A276S+A364G+S388K; A25W+V45L+A117S+L149R+S214Q+V258P+A276S+A364G+S388K;

V45L+A117S+L149R+S214Q+V258P+A276S+A364G+S388K;

V45L+A117S+L149R+S214Q+V258P+A276S+A364G+S388K+N476N;

A25W+V45L+L149R+S214E+V258P+A276Q+A364G;

V45L+A117S+L149R+S214Q+V258P+A276S+A364G+S388K+N476D;

V45L+L149R+S214Q+V258P+A276S+L281V+A364G;

V45L+A93S+L149R+A252S+H334D+A340G;

V45L+A93S+L149R+A252S+H334D;

V45L+A93S+L149R+A252S+H334D;

V45L+L149R+A252S+H334D;

V45L+N127D+L149R+V258P+A276S+A364G+S388K;

V45L+L149R+V258P+A276S+H358T+A364G+S388K;

V45L+L149R+V258P+A276S+A364G+S388K; D9E+V45L+A117S+L149R+S214Q+V258P+A276S+A364G+S388K;

V45L+A117S+L149R+S214Q+V258P+A276S+A364G+S388K+S481S;

V45L+L149R+N168G+V258P+A276S+A364G;

V45L+L149R+T180Q+V258P+A276S+H358T+A364G+S388K;

V45L+L149R+G187L+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K; A25W+V45L+L149R+V258P+A276S+A364G+S388K;

V45L+L149R+S214Q+V258P+A276S+H358T+A364G;

V45L+L149R+S173Y+V258P+A276S+A364G;

V45L+A117S+L149R+S214Q+H219V+V258P+A276S+A364G+S388K;

V45L+A93S+L149R+A252S+H334D;

V45L+A93S+L149R+A252S+H334D+A340G;

V45L+L149R+S190E+D446T+R459V; A25W+V45L+L149R+V258P+A276S+H358T; A25W+V45L+S92D+L149R+S214E+V258P+A276S+A364G; A25W+V45L+L149R+S214E+V258P+A276S+V350L+A364G; A25W+V45L+L149R+S214Q+V258P+A276S+A364G;

V45L+L149R+N168G+V258P+A276S+A364G+S388K;

V45L+A117S+L149R+V258P+A276S+A364G+S388K; A25W+V45L+L149R+V258P+A276S+H358T+A364G;

V45L+L149R+V258P+N260H+A276S+A364G+S388K;

V45L+L149R+T215I+V258P+A276S+A364G+S388K; A25W+V45L+L149R+V258P+A276Q+A364G;

V45L+T70R+A117S+L149R+C161L+S214Q+V258P+A276S+L281V+H358D+A364G+S388K+G394A+D470T+R483V;

A25W+V45L+L149R+S214E+V258P+A276S;

V45L+A117S+L149R+S214Q+V258P+A276S+A286S+H358D+A364G+S388K;

V45L+L149R+T211H+S214Q+V258P+A276S+A364G+S388K;

V45L+Q61K+L149R+V258P+A276S+H358T+A364G;

V45L+L149R+V258P+A276S+Q314A+H358T+A364G;

V45L+L149R+T180Q+V258P+A276S+A364G+S388K;

V45L+L149R+V258P+A276S+Q314A+A364G+S388K; A25W+V45L+L149R+V258P+A276S+A286S+A364G;

V45L+L149R+S190E+D446T;

V45L+L149R+V258P+A276S+A364G;

V45L+L149R+A170S+V258P+A276S+A364G;

V45L+L149R+V258P+A276Q+A364G;

V45L+T70R+A117S+L149R+S214Q+V258P+A276S+L281V+H358D+A364G+S388K+G394A+D470T+R483V+C522A;

V45L+L149R+H219I+V258P+A276S+H358T+A364G;

V45L+L149R+V258P+A276S+V350L+A364G+S388K; D11E+V45L+L149R+V258P+A276S+A364G+S388K;

V45L+D113T+L149R+V258P+A276S+A364G+S388K;

V45L+L149R+G187K+V258P+A276S+A364G+S388K; M28P+V45L+L149R+V258P+A276S+A364G+S388K;

V45L+Q61R+A117S+L149R+S214Q+V258P+A276S+H358D+A364G+S388K+D470T+R483V; A25W+V45L+L149R+S214Q+V258P+A276S;

V45L+A117S+L149R+S214Q+T215I+V258P+A276S+A364G+S388K; A25W+V45L+L149R+V258P+A276S+S388K; A25W+V45L+L149R+S214E+H219I+V258P+A276S+A364G;

V45L+A93S+L149R+V234P+A252S+H334D;

V45L+L149R+T211H+V258P+A276S+H358T+A364G+S388K; A25W+V45L+A117S+L149R+S214E+V258P+A276S+A364G;

V45L+L149R+S173Y+V258P+A276S+A364G+S388K; A25W+V45L+L149R+V258P+A276S+Q314A+A364G;

V45L+L149R+G187L+V258P+A276S+H358T+A364G;

V45L+L149R+T215V+V258P+A276S+H358T+A364G;

V45L+T70R+L149R+V258P+A276S+A364G+S388K;

V45L+N127D+L149R+V258P+A276S+A364G;

V45L+A117S+L149R+S214Q+V258P+A276S+A364G+S388K+Q496R;

V45L+L149R+W250Y+V258P+A276S+A364G;

V45L+L149R+V258P+A276S+V350L+H358T+A364G; D9E+V45L+L149R+V258P+A276S+H358T+A364G;

V45L+L149R+T211H+V258P+A276S+H358T+A364G; A25W+V45L+L149R+W250Y+A276Q+A364G;

V45L+L149R+V258P+A276S+L281V+A364G+S388K;

V45L+L149R+V258P+A276S+S282N+H358T+A364G;

V45L+L149R+T215I+V258P+A276S+H358T+A364G; A25W+V45L+L149R+S173Y+V258P+A276S+A364G;

V45L+Q61K+L149R+V258P+A276S+A364G+S388K;

V45L+L149R+T180Q+V258P+A276S+A364G;

V45L+A117S+L149R+A170S+S214Q+V258P+A276S+A364G+S388K;

V45L+Q61R+A117S+L149R+S214Q+V258P+A276S+A364G+S388K; A25W+V45L+L149R+S214E+T215V+V258P+A276S+A364G;

V45L+A117S+L149R+S214Q+V258P+A276S+A364G+S388K+V497P;

V45L+L149R+V258P+A276S+Q314A+A364G;

V45L+A117S+L149R+V258P+A276S+A364G;

V45L+A117S+L149R+V258P+A276S+H358T+A364G+S388K;

V45L+L149R+N168G+V258P+A276S+H358T+A364G+S388K; A25W+V45L+Q61K+L149R+V258P+A276S+A364G;

V45L+L149R+S214E+V258P+A276S+L281V+A364G;

V45N+L149R+V258P+A276S+A364G+S388K;

V45L+L149R+V258P+A276S+A286E+H358T+A364G; D9E+V45L+L149R+V258P+A276S+A364G+S388K;

V45L+S92D+L149R+V258P+A276S+A364G+S388K;

V45L+A117S+L149R+T180Q+S214Q+V258P+A276S+A364G+S388K;

V45L+A117S+L149R+S214Q+V258P+A276S+A364G+S388H;

V45L+A117S+L149R+S214Q+T215V+V258P+A276S+A364G+S388K;

V45L+A117S+L149R+S214Q+V258P+A276S+V350L+A364G+S388K; A25W+V45L+L149R+S214E+T215I+V258P+A276S+A364G;

V45L+N127D+L149R+V258P+A276S+H358T+A364G+S388K; A25W+V45L+L149R+V258P+A276S+A286E+A364G;

V45L+L149R+N168G+V258P+A276S+L281V+A364G;

V45L+Q61R+L149R+V258P+A276S+H358T+A364G;

V45L+L149R+G187S+V258P+A276S+A364G+S388K;

V45L+L149R+V258P+A276S+L281V+Q314A+A364G;

V45L+L149R+V258P+A276S+L281V+H358T+A364G;

V45L+L149R+A170S+V258P+A276S+H358T+A364G;

V45L+L149R+A170S+V258P+A276S+A364G+S388K;

V45L+L149R+T215V+V258P+A276S+A364G+S388K;

V45L+L149R+V258P+A276Q+L281V+A364G;

V45L+T70R+A117S+L149R+S214Q+C243S+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V+C522V; A25W+V45L+L149R+V258P+A276S+Q314A;

V45L+L149R+V258P+A276Q+H358T+A364G+S388K; A25W+V45N+L149R+S214E+V258P+A276S+A364G;

V45L+L149R+V258P+A276S+A286E+A364G;

V45L+L149R+V258P+A276S+A286S+H358T+A364G; A25W+V45L+L149R+G187S+V258P+A276S+A364G;

V45L+L149R+T180Q+V258P+A276S+L281V+A364G; A25W+V45L+L149R+A276Q+L281V+A364G;

V45L+S92D+L149R+V258P+A276S+L281V+A364G;

V45L+Q61R+L149R+V258P+A276S+L281V+A364G;

V45L+L149R+A170S+V258P+A276S+L281V+A364G; D11E+A25W+V45L+L149R+V258P+A276S+A364G; A25W+V45L+Q61R+L149R+V258P+A276S+A364G; A25W+V45L+L149R+T215V+V258P+A276S+A364G; D9E+V45L+L149R+V258P+A276S+L281V+A364G; A25W+V45L+L149R+V258P+A276S+V350L+A364G;

V45L+Q61K+A117S+L149R+S214Q+V258P+A276S+A364G+S388K;

V45L+A117S+L149R+S214Q+V258P+A276S+A364G+S388K+Y391V;

V45L+A117S+L149R+N168G+S214Q+V258P+A276S+A364G+S388K;

V45L+D113T+A117S+L149R+S214Q+V258P+A276S+A364G+S388K; A25W+V45L+L149R+V258P+A276S+A286E; A25W+V45L+L149R+V258P+A276S+L281V; A25W+V45L+D113T+L149R+V258P+A276S;

V45L+L149R+H219V+V258P+A276S+L281V+A364G;

V45L+L149R+T180Q+V258P+A276S+H358T+A364G;

V45L+L149R+T215V+V258P+A276S+L281V+A364G; A25W+V45L+L149R+V258P+A276S+S282N+A364G; A25W+V45L+L149R+T215I+V258P+A276S+A364G; A25W+V45L+L149R+H219I+V258P+A276S+A364G;

V45L+A117S+L149R+V258P+A276S+L281V+A364G;

D9E+A25W+V45L+L149R+V258P+A276S+A364G;

V45L+L149R+S214Y+V258P+A276S+A364G+S388K; A25W+V45L+L149R+T180Q+V258P+A276S+A364G; A25W+V45L+L149R+V258P+A276S+L281V+A364G;

V45L+N127D+L149R+V258P+A276S+L281V+A364G;

V45L+L149R+S173Y+V258P+A276S+H358T+A364G;

V45L+Q61R+L149R+V258P+A276S+A364G+S388K; M28P+V45L+A117S+L149R+S214Q+V258P+A276S+A364G+S388K;

V45L+A117S+L149R+S214Q+V258P+A276S+A364G+S388A;

V45N+A117S+L149R+S214Q+V258P+A276S+A364G+S388K;

V45L+A117S+L149R+S214Q+V258P+A276S+A364G+S388K+T477K; A25W+V45L+L149R+T180Q+V258P+A276S; A25W+V45L+L149R+V258P+A276S+A286S; A25W+V45L+L149R+G187S+S214E+V258P+A276S+A364G;

V45L+L149R+V258P+A276S+L281V+A286E+A364G; A25W+V45L+L149R+W250Y+V258P+A276S+A364G; A25W+V45L+L149R+V258P+N260H+A276S+A364G;

V45L+L149R+V258P+A276S+L281V+V350L+A364G; A25W+V45L+L149R+A170S+V258P+A276S+A364G;

V45L+L149R+V258P+A276S+L281V+A286S+A364G;

V45L+L149R+V258P+A276S+L281V+S282N+A364G; D11E+V45L+L149R+V258P+A276S+L281V+A364G;

V45L+Q61K+L149R+V258P+A276S+L281V+A364G;

V45L+L149R+T215I+V258P+A276S+L281V+A364G; A25W+V45L+S92D+L149R+V258P+A276S+A364G;

V45L+L149R+S214Y+V258P+A276S+L281V+A364G;

V45L+L149R+T211H+V258P+A276S+L281V+A364G; A25W+V45L+L149R+G187L+V258P+A276S+A364G;

V45L+S92D+L149R+V258P+A276S+H358T+A364G;

V45L+L149R+G187L+A276Q+L281V+A364G;

V45L+L149R+A276Q+L281V+V350L+A364G;

V45L+L149R+A276Q+L281V+A364G+S388K;

V45L+A117S+L149R+S214Y+V258P+A276S+A364G+S388K; A25W+V45L+S92D+L149R+V258P+A276S; A25W+V45L+L149R+G187L+V258P+A276S; A25W+V45L+L149R+W250Y+V258P+A276S;

V45L+D113T+L149R+V258P+A276S+A364G;

V45L+L149R+G187K+V258P+A276S+H358T+A364G;

V45L+L149R+N163H+V258P+A276S+L281V+A364G; A25W+V45L+T70R+L149R+V258P+A276S+A364G;

V45L+L149R+S173Y+V258P+A276S+L281V+A364G;

V45L+L149R+G187L+V258P+A276S+L281V+A364G;

V45L+L149R+G187K+V258P+A276S+L281V+A364G;

V45L+L149R+W250Y+V258P+A276S+H358T+A364G;

V45L+L149R+T211H+V258P+A276S+A364G+S388K;

V45L+L149R+N163H+V258P+A276S+A364G+S388K;

V45L+T70R+L149R+A276Q+L281V+A364G;

V45L+S92D+L149R+A276Q+L281V+A364G;

V45L+D113T+L149R+A276Q+L281V+A364G;

V45L+L149R+A276Q+L281V+Q314A+A364G;

V45L+A117S+L149R+S214Q+V258P+A276S+A364G+S388K+S389V; A25W+V45L+Q61K+L149R+V258P+A276S; D9E+A25W+V45L+L149R+V258P+A276S;

V45L+T70R+L149R+V258P+A276S+A364G;

V45L+L149R+V258P+A276S+A286S+A364G+S388K; A25W+V45L+L149R+S214Y+V258P+A276S+A364G; A25W+V45L+D113T+L149R+V258P+A276S+A364G;

V45L+T70R+L149R+V258P+A276S+L281V+A364G; A25W+V45L+N127D+L149R+V258P+A276S+A364G;

V45L+T70R+L149R+V258P+A276S+H358T+A364G;

V45L+L149R+N163H+V258P+A276S+A364G;

V45L+L149R+T180Q+A276Q+L281V+A364G;

V45L+L149R+A276Q+L281V+A286S+A364G; A25W+V45L+L149R+A170S+V258P+A276S; A25W+V45L+T70R+L149R+V258P+A276S; A25W+V45L+L149R+S214E+V258P+A276S+A286S+A364G;

V45L+L149R+V258P+A276S+A286S+H358T+A364G+S388K;

V45L+L149R+H219I+V258P+A276S+L281V+A364G; A25W+V45L+A117S+L149R+V258P+A276S+A364G; A25W+V45L+L149R+N168G+V258P+A276S+A364G;

V45L+L149R+N168G+A276Q+L281V+A364G;

V45L+L149R+S173Y+A276Q+L281V+A364G;

V45L+L149R+W250Y+A276Q+L281V+A364G;

V45L+A117S+L149R+S173Y+S214Q+V258P+A276S+A364G+S388K;

A25W+V45L+L149R+A276Q+A286E+A364G; A25W+V45L+L149R+A276Q+A364G+S388K;

V45L+N127D+L149R+V258P+A276S+H358T+A364G; A25W+V45L+L149R+V258P+A276S;

V45L+N127D+L149R+A276Q+L281V+A364G;

V45L+L149R+A276Q+L281V+A286E+A364G;

V45L+A117S+L149R+S214Q+V258P+A276S+A364G+S388K+V500T; A25W+V45L+A117S+L149R+V258P+A276S; A25W+V45L+L149R+H219V+V258P+A276S+A364G;

V45L+L149R+S173Y+V258P+A276S+H358T+A364G+S388K; D11E+V45L+L149R+V258P+A276S+H358T+A364G;

V45L+A79C+L149R+V258P+A276S+N355G+A364G+S388K; A24K+V45L+L149R+V258P+A276S+A364G;

V45L+L149R+V258P+A276S+S282N+A364G;

V45L+A117S+L149R+A276Q+L281V+A364G;

V45L+L149R+N260H+A276Q+L281V+A364G;

V45L+A117S+L149R+T211H+S214Q+V258P+A276S+A364G+S388K; A25W+V45L+L149R+N168G+V258P+A276S; A25W+V45L+L149R+G187S+V258P+A276S; A25W+V45L+L149R+V258P+A276S+V350L; A25W+V45L+Q61R+L149R+V258P+A276S; A25W+V45L+L149R+G187L+S214E+V258P+A276S+A364G;

V45L+L149R+G187S+V258P+A276S+L281V+A364G; A25W+V45L+L149R+N163H+V258P+A276S+A364G; A25W+V45L+L149R+G187K+V258P+A276S+A364G;

V45N+L149R+V258P+A276S+L281V+A364G;

V45L+L149R+A276Q+L281V+A364G;

V45L+L149R+V258P+N260H+A276S+H358T+A364G;

V45L+L149R+A276Q+S282N+A364G;

V45L+S92D+A117S+L149R+S214Q+V258P+A276S+A364G+S388K; A25W+V45L+L149R+V258P+A276S+S282N; A25W+V45L+L149R+S173Y+V258P+A276S; A25W+V45L+L149R+A276Q+A286S+A364G;

V45L+D113T+L149R+V258P+A276S+L281V+A364G;

V45L+L149R+S214Q+V258P+A276S+L281V+A364G+S388K;

V45L+D113T+L149R+V258P+A276S+H358T+A364G; A25W+V45L+L149R+T215V+V258P+A276S;

V45L+L149R+N168G+V258P+A276S+H358T+A364G;

V45L+A117S+L149R+S214Q+V258P+A276S+A364G+S388K+A472L;

V45L+A117S+L149R+S214Q+V258P+A276S+A364G+S388K+S473K; A25W+V45L+L149R+T215I+V258P+A276S; A25W+V45L+L149R+T211H+V258P+A276S; A25W+V45L+L149R+V258P+A276Q;

V45L+L149R+S214Q+T215V+V258P+A276S+A364G+S388K;

V45L+L149R+N163H+V258P+A276S+H358T+A364G+S388K;

V45L+A117S+L149R+S214Q+V258P+A276S+A364G+S388K+A499L;

V45L+L149R+V258P+A276S+V350L+A364G;

V45L+A79C+A117S+L149R+V258P+A276S+A364G+S388K; A25W+V45L+D113T+L149R+A276Q+A364G; A25W+V45N+L149R+V258P+A276S+A364G;

V45L+T70R+L149R+A276Q+A364G; A25W+V45L+L149R+H219I+V258P+A276S;

V45L+A117S+L149R+S214Q+V258P+A276S+A364G+S388K+V498I; M28P+V45L+L149R+V258P+A276S+A364G; A25W+V45L+T70R+L149R+A276Q+A364G; A25W+V45L+L149R+A276Q+Q314A+A364G;

V45L+L149R+A170S+V258P+A276S+H358T+A364G+S388K; A25W+V45L+L149R+S214Y+V258P+A276S;

V45L+L149R+S214Y+V258P+A276S+H358T+A364G;

V45L+L149R+A276Q+L281V+H358T+A364G; A25W+V45L+L149R+H219V+V258P+A276S;

V45L+A117S+L149R+N163H+S214Q+V258P+A276S+A364G+S388K; D11E+A25W+V45L+L149R+V258P+A276S;

V45L+A117S+L149R+S214Q+V258P+A276S+A364G+S388K+N527H; A25W+V45L+A117S+L149R+A276Q+A364G;

V45L+L149R+V258P+L281V+A364G; A25W+V45L+L149R+G187L+A276Q+A364G;

V45N+L149R+V258P+A276S+H358T+A364G;

V45L+A117S+L149R+S214Q+V258P+A276S+N355G+H358D+A364G+S388K+D470T+R483V;

V45L+A117S+L149R+S214Q+V258P+A276S+H358D+A364G+S388H+D470T+R483V; A25W+V45L+L149R+A276Q+A364G;

V45L+A117S+L149R+S214Q+V258P+A276S+H358D+A364G+S388K+D470T+R483V+V497E;

A25W+V45L+L149R+N163H+A276Q+A364G;

A25W+V45L+L149R+S173Y+A276Q+A364G;

A25W+V45L+L149R+A276Q+S282N+A364G;

A25W+V45L+L149R+A276Q+H358T+A364G;

A25W+V45L+L149R+T180Q+A276Q+A364G;

V45L+A117S+L149R+V258P+A276S+H358T+A364G;

V45L+L149R+A276Q+Q314A+A364G;

V45L+L149R+G187K+V258P+A364G;

V45L+L149R+A276Q+A286E+A364G; A25W+V45L+N127D+L149R+A276Q+A364G; A25W+V45L+L149R+A170S+A276Q+A364G; A25W+V45L+N127D+L149R+V258P+A276S;

V45L+A117S+L149R+S214Q+V258P+A276S+A364G+S388K+T477V;

V45L+A117S+L149R+S214Q+V258P+A276S+A364G+S388K+G503L; A25W+M28P+V45L+L149R+V258P+A276S+A364G;

V45L+L149R+A276Q+H358T+A364G; A25W+V45L+L149R+N168G+A276Q+A364G; A25W+V45L+L149R+N260H+A276Q+A364G;

V45L+A117S+L149R+G187L+S214Q+V258P+A276S+A364G+S388K;

V45L+A117S+L149R+N163H+S214Q+V258P+A276S+H358D+A364G+S388K+D470T+L530R;

V45L+A117S+L149R+S214Q+T215K+V258P+A276S+A364G+S388K; A25W+V45L+L149R+G187K+V258P+A276S; A25W+M28P+V45L+L149R+V258P+A276S; A25W+V45N+L149R+V258P+A276S;

V45L+A117S+L149R+S214Q+V258P+A276S+N355G+H358D+A364G+S388K+D470T;

V45L+N127D+L149R+A276Q+A364G;

V45L+L149R+W250Y+A276Q+A364G;

V45L+L149R+W250Y+V258P+A364G;

V45L+L149R+N163H+A276Q+L281V+A364G;

V45L+A117S+L149R+S214Q+V258P+A276S+A364G+Q385C+S388K; A25W+V45L+L149R+S214E+T215K+V258P+A276S+A364G; M28P+V45L+L149R+V258P+A276S+L281V+A364G;

V45L+L149R+A276Q+A364G+S388K;

A25W+V45L+L149R+V258P+A364G;

M28P+V45L+L149R+V258P+A276S+H358T+A364G;

V45L+A117S+L149R+S214Q+V258P+A276S+N355G+H358D+A364G+S388K;

V45L+T70R+A117S+L149R+S214Q+V258P+A276S+L281V+H358D+A364G+S388K+G394A+D470T+R483V+C540V;

V45L+L149R+N168G+A276Q+A364G;

V45L+A117S+L149R+A276Q+A364G;

V45L+L149R+N163H+V258P+A276S+H358T+A364G;

V45L+L149R+T215K+V258P+A276S+H358T+A364G; A24K+V45L+L149R+A276Q+A364G;

V45L+L149R+A276Q+A286S+A364G;

V45L+L149R+V258P+A364G;

V45L+L149R+H219I+V258P+A276S+A364G+S388K;

V45L+L149R+A170S+A276Q+A364G;

V45L+L149R+T180Q+A276Q+A364G;

V45L+L149R+A276Q+V350L+A364G;

V45L+L149R+V258P+S282N+A364G;

V45L+L149R+A276Q+A364G;

V45L+L149R+A276S+A364G;

V45L+L149R+T215K+V258P+A276S+A364G+S388K;

V45L+A117S+L149R+S214Q+V258P+A276S+N355G+A364G+S388K+D470T;

V45L+L149R+V258P+A276S; A25W+V45L+L149R+A276S+A364G;

V45L+L149R+G187S+V258P+A364G;

V45L+S92D+L149R+V258P+A276S+H358T+A364G+S388K; A25W+L149R+V258P+A276S+A364G;

V45L+L149R+G187L+A276Q+A364G; A25W+V45L+V258P+A276S+A364G;

V45L+L149R+N260H+A276Q+A364G;

V45L+L149R+W250Y+V258R+A364G;

V45L+L149R+N163H+A276Q+A364G;

V45L+S92D+L149R+A276Q+A364G;

V45L+L149R+S173Y+A276Q+A364G;

V45L+S92D+L149R+V258P+A364G;

V45L+L149R+W250Y+V258P+A276S+L281V+A364G;

V45L+L149R+L281V+A364G;

V45L+D113T+L149R+A276Q+A364G;

L149R+V258P+A276S+A364G;

V45L+L149R+G187S+V258R+A364G;

V45L+V258P+A276S+A364G;

V45L+L149R+V258R+A364G+S388K; A25W+V45L+L149R+A364G;

V45L+L149R+G187K+V258R+A364G;

V45L+L149R+V258P;

V45L+L149R+V258R+S282N+A364G;

V45L+L149R;

V45L+L149R+V258R+L281V+A364G;

V45L+L149R+A364G;

V45L+L149R+S214Q+V258P+A276S+N355G+A364G+S388K;

V45L+S92N+L149R+V258P+A364G;

V45L+A117S+L149R+S214Q+V258P+A276S+A286E+H358D+A364G+S388K; A25W+V45L+L149R+S214E+V258P+A276S+N355G+A364G;

V45L+L149R+G187L+V258P+A276S+A364G;

V45L+L149R+V258R+A364G;

V45L+L149R+V258P+A364G+S388K; A25W+V45L+L149R+A276Q+V350L+A364G;

V45L+L149R+H219V+V258P+A276S+A364G+S388K;

V45L+L149R+G187K+A364G;

V45L+L149R+G187S+A364G;

V45L+A117S+L149R+S214Q+V258P+A276S+N355G+A364G+S388K;

V45L+A117S+L149R+S214Q+V258P+A276S+H358D+A364G+Y386G+S388K+D470T+R483V;

V45L+L149R+W250Y;

V45L+S92N+L149R+V258R+A364G; A25W+V45L+L149R+V258P+A276S+N355G+A364G;

V45L+L149R+V258P+A276S+N355G+A364G+S388K;

V45L+L149R+G187S;

V45N+L149R;

V45L+L149R+S282N; L149R+A364G; Y386G;

V258P+A276Q;

V258P+A276S;

V45L+A117S+L149R+S214Q+V258P+A276S+H358D+A364G+S388K+G394D+D470T+R483V;

A25W+L149R;

V45L+A276S;

V45L+S92D+L149R;

A25W+A276S;

Q385C;

V45L+L149R+W250Y+A364G;

V45L+A276Q;

V45L+V258P;

V45L+A117S+L149R+S214Q+V258P+A276S+H358D+A364G+Y386G+S388K;

A25W+V258P;

A25W+A276Q;

L149R+W250Y;

V45L+L149R+V258P+A276S+L281V+N355G+A364G;

V45L+S388K;

V45L+L149R+V258P+A276S+N355G+H358T+A364G;

V45L+A364G;

V45N+V258P;

A276S;

V258P+S388K;

A25W+V45L;

A276Q+S388K;

A276C;

A276Q;

A276F;

W250Y+A276Q;

A79C;

A276S+S388K;

A276Q+L281V;

V45L+L149R+V258P+A276S+N355G+A364G;

A276N;

W250Y+A276S;

A25W+S388K;

A276S+L281V;

G187S+A276Q;

A25W+V45L+L149R+V258P+A276S+N355G;

V45L;

S92D+A276S;

S19T;

L149R+V258R;

V45N+S388K;

A25W+V45L+S92D+L149R+A276Q+A364G;

V45L+A117S+L149R+S214Q+V258P+A276S+A364G+S388K+D470T+S501E H358D;

A25W+A364G;

V258P+L281V;

L149R;

V45L+L149R+H219V+V258P+A276S+H358T+A364G;

A25W;

V258R+A276S;

V45N;

G187S+A276S;

G187K+A276S;

I469T;

V45L+S92D;

V45L+L281V;

V45N+W250Y;

V258R+A276Q;

V45L+V258R;

W250Y+S388K;

A25W+V45L+L149R+A276Q+N355G+A364G;

V45N+G187K;

A25W+V45L+L149R+S214E+V258P+A276S+Q314A+A364G;

S388A;

A276T;

A25W+V258R;

G187K+S388K;

A25W+S92D;

L281V+S388K;

V45N+V258R;

S388H;

S92D;

D525L;

S214Q;

L281V+A364G;

A364G;

S388L;

S388R;

S473G;

S92D+A364G;

A25W+G187S;

A25W+G187K;

W250Y+V258P;

S92D+V258P;

S389V;

A20H;

W495E;

G187K+W250Y;

Y386Y;

V393H;

G187L;

Q61K;

V45N+S92D;

G187S+A364G;

G187S+L281V;

G187S;

A286E;

A276Y;

V258P;

Q496E;

G394A;

V393R;

G187S+S388K;

T366V;

S389S;

S92N;

N527H;

Q384A;

V497D;

A18V;

Q496Q;

Q496R;

S214E;

P362P;

S389H;

V258R;

A276D;

L281V;

V45I;

A117C;

N395N;

Y391Y;

A24H;

A472I;

D470K;

S473K;

S473S;

W495I;

V497E;

A24K;

V45T;

L471R;

A472L;

A20P;

N395D;

G394D;

G394Q;

G187S+V258R;

S92D+G187S;

V45C;

N476N;

G207G;

A364A;

D470A;

N474G;

D470V; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by TSA of at least 0.5° C. compared to the xylanase of SEQ ID NO: 1.

In one aspect, the present invention relates to xylanase variants comprises at least one of the following substitutions or combinations of substitutions:

V45L+Q61K+T70R+A117S+L149R+S214Q+V258P+A276S+L281V+A286E+H358D+A364G+Q384A+S388K+V393H+D470T+R483V;

V45L+Q61K+T70R+A117S+L149R+S214Q+V258P+A276S+L281V+H358D+A364G+Q384A+S388K+G394Q+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+A286E+H358D+A364G+Q384A+S388K+V393H+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+A79C+A117S+L149R+S214Q+V258P+A276S+H358D+A364G+S388K+D470T+L530R;

V45L+A79C+A117S+L149R+S214Q+V258P+A276S+H358D+A364G+S388K+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+Q61K+A117S+L149R+S214Q+V258P+A276S+L281V+H358D+A364G+S388K+G394A+D470T+R483V+L530R;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+Q61K+A117S+L149R+S214Q+V258P+A276S+L281V+H358D+A364G+S388K+G394A+D470T+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+D470T+R483V+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+D470T+R483V+L530R;

V45L+A117S+L149R+G187K+S214Q+V258P+A276S+A286E+H358D+A364G+Q384A+S388K+V393H+D470T+R483V;

V45L+A117S+L149R+G187K+S214Q+V258P+A276S+A286E+H358D+A364G+Q384A+S388K+N395D+D470T+R483V;

V45L+A117S+L149R+G187K+S214E+T215I+V258P+A276S+A286E+H358D+A364G+S388K+V393H+D470T+R483V;

V45L+A117S+L149R+G187K+S214E+T215I+V258P+A276S+A286E+H358D+A364G+S388K+N395D+D470T+R483V;

V45L+A117S+L149R+S214Q+V258P+A276S+H358D+A364G+Q385C+S388K+D470T+R483V;

V45L+Q61K+A117S+L149R+S214Q+V258P+A276S+H358D+A364G+S388K+D470T+R483V;

V45L+A79C+A117S+L149R+S214Q+V258P+A276S+A364G+S388K+D470T+R483V;

V45L+A117S+L149R+S214Q+V258P+A276S+L281V+H358D+A364G+S388K+D470T+R483V;

V45L+A117S+L149R+S214Q+V258P+A276S+H358D+A364G+T366I+S388K+D470T+R483V;

V45L+T70R+A117S+L149R+S214Q+V258P+A276S+L281V+H358D+A364G+S388K+G394A+D470T+R483V;

V45L+A117S+L149R+S214Q+V258P+A276S+H358D+A364G+Q384A+S388K+D470T+L530R;

V45L+Q61K+A117S+L149R+S214Q+V258P+A276S+H358D+A364G+S388K+D470T+L530R;

V45L+T70R+A117S+L149R+S214Q+V258P+A276S+H358D+A364G+S388K+D470T+R483V;

V45L+A117S+L149R+S214Q+V258P+A276S+H358D+A364G+S388K+V393H+D470T+R483V;

V45L+A117S+L149R+S214Q+V258P+A276S+H358D+A364G+S388K+G394A+D470T+R483V;

V45L+A117S+L149R+S214Q+V258P+A276S+H358D+A364G+S388K+V393H+D470T+L530R;

V45L+A117S+L149R+S214Q+V258P+A276S+A286E+H358D+A364G+S388K+D470T+R483V;

D11E+V45L+A117S+L149R+S214Q+V258P+A276S+H358D+A364G+S388K+D470T+R483V;

V45L+T70R+A117S+L149R+S214Q+V258P+A276S+L281V+H358D+A364G+S388K+G394A+C468V+D470T+R483V;

V45L+A79C+A117S+L149R+S214Q+V258P+A276S+H358D+A364G+S388K+D470T;

V45L+A79C+L149R+S214E+V258P+A276S+A364G+S388K;

V45L+A117S+L149R+G187L+S214Q+V258P+A276S+H358D+A364G+S388K+D470T+L530R;

V45L+A117S+L149R+S214Q+V258P+A276S+H358D+A364G+Q384A+S388K+D470T+R483V;

V45L+A117S+L149R+G187K+S214Q+V258P+A276S+H358D+A364G+S388K+D470T+R483V;

V45L+A117S+L149R+S214Q+V258P+A276S+L281V+H358D+A364G+S388K+D470T+L530R;

V45L+A117S+L149R+S214E+V258P+A276S+H358D+A364G+S388K+D470T+R483V;

V45L+A117S+L149R+G187L+S214Q+V258P+A276S+H358D+A364G+S388K+D470T+R483V;

V45L+A117S+L149R+S214Q+V258P+A276S+H358D+A364G+S388K+G394Q+D470T+R483V;

V45L+A117S+L149R+S214Q+V258P+A276S+H358D+A364G+S388K+G394A+D470T+L530R;

V45L+A117S+L149R+S214Q+V258P+A276S+H358D+A364G+T366V+S388K+D470T+L530R;

V45L+A117S+L149R+S214Q+V258P+A276S+H358D+A364G+S388K+G394Q+D470T+L530R;

V45L+A117S+L149R+S214Q+V258P+A276S+S282N+H358D+A364G+S388K+D470T+R483V;

V45L+A117S+L149R+S214Q+V258P+A276S+H358D+A364G+S388K+N395D+D470T+R483V;

V45L+A117S+L149R+S214Q+V258P+A276S+H358D+A364G+S388K+D470T+N476D+R483V;

V45L+A117S+L149R+S214Q+V258P+A276S+V350L+H358D+A364G+S388K+D470T+R483V;

V45L+A117S+L149R+S214Q+V258P+A276S+H358D+A364G+S388K+D470T+T477K+R483V;

V45L+A117S+L149R+S214Q+V258P+A276S+H358D+A364G+S388K+D470T+R483V+Q496R;

V45L+T70R+A117S+L149R+S214Q+V258P+A276S+L281V+H358D+A364G+S388K+G394A+C468A+D470T+R483V;

V45L+A117S+L149R+S214Q+V258P+A276S+H358D+A364G+S388K+D470T+R483V+W495E;

V45L+A117S+L149R+S214Q+V258P+A276S+A286E+H358D+A364G+S388K+D470T+L530R;

V45L+A117S+L149R+G187K+S214Q+V258P+A276S+H358D+A364G+S388K+D470T+L530R;

V45L+A117S+L149R+S214Q+V258P+A276S+H358D+A364G+S388K+Y391V+D470T+R483V;

V45L+A117S+L149R+S214Q+V258P+A276S+H358D+A364G+S388K+D470T+R483V;

D9E+V45L+A117S+L149R+S214Q+V258P+A276S+H358D+A364G+S388K+D470T+L530R;

V45L+A117S+L149R+S214Q+V258P+A276S+H358D+A364G+S388K+D470T+T477V+R483V;

V45L+A117S+L149R+S214Q+V258P+A276S+H358D+A364G+S388K+D470T+N476N+R483V;

V45L+A117S+L149R+S214Q+V258P+A276S+H358D+A364G+S388K+V393R+D470T+R483V;

V45L+A117S+L149R+S214Q+V258P+A276S+H358D+A364G+S388K+D470T+T480L+L530R;

V45L+A117S+L149R+S214Q+V258P+A276S+H358D+A364G+S388K+D470T+S481A+L530R;

V45L+A117S+L149R+S214Q+W250Y+V258P+A276S+H358D+A364G+S388K+D470T+R483V;

V45L+A117S+L149R+S214Q+V258P+A276S+H358D+A364G+S388K+D470T+S481S+R483V;

V45L+A117S+L149R+S173Y+S214Q+V258P+A276S+H358D+A364G+S388K+D470T+R483V;

V45L+T70R+A117S+L149R+S214Q+C243V+V258P+A276S+L281V+H358D+A364G+S388K+G394A+D470T+R483V;

V45L+A117S+L149R+T180Q+S214Q+V258P+A276S+H358D+A364G+S388K+D470T+L530R;

D11E+V45L+A117S+L149R+S214Q+V258P+A276S+H358D+A364G+S388K+D470T+L530R;

V45L+A117S+L149R+S214E+V258P+A276S+H358D+A364G+S388K+D470T+L530R N476D+L530R;

V45L+A117S+L149R+S214Q+V258P+A276S+A286S+H358D+A364G+S388K+D470T+R483V;

V45L+A117S+L149R+S214Q+V258P+A276S+H358D+A364G+S388K+D470T+R483V+Q496Q;

V45L+A117S+L149R+S214Q+V258P+A276Q+H358D+A364G+S388K+D470T+R483V;

V45L+A117S+L149R+S214Q+V258P+A276S+H358D+A364G+S388K+D470T+R483V+Q496E;

V45L+Q61R+A117S+L149R+S214Q+V258P+A276S+H358D+A364G+S388K+D470T+L530R;

V45L+A117S+L149R+S214Q+V258P+A276S+Q314A+H358D+A364G+S388K+D470T+L530R;

V45L+A117S+L149R+S214Q+V258P+A276Q+H358D+A364G+S388K+D470T+L530R;

V45L+A117S+L149R+S214Q+V258P+A276S+H358D+A364G+S388K+D470T+L478P+R483V;

A25W+V45L+A117S+L149R+S214Q+V258P+A276S+H358D+A364G+S388K+D470T+R483V;

V45L+A117S+L149R+T180Q+S214Q+V258P+A276S+H358D+A364G+S388K+D470T+R483V;

V45L+A117S+L149R+S214Q+V258P+A276S+H358D+A364G+S388K+D470T+R483V+L484K;

V45L+A117S+L149R+S214Q+V258P+A276S+H358D+A364G+S388K+D470T+N474G+L530R;

V45L+A117S+L149R+S214Q+V258P+A276S+H358D+A364G+S388K+N395D+D470T+L530R;

V45L+A117S+L149R+S214Q+V258P+A276S+H358D+A364G+T366V+S388K+D470T+R483V;

V45L+A117S+L149R+N168G+S214Q+V258P+A276S+H358D+A364G+S388K+D470T+R483V;

V45L+A117S+L149R+S214Q+V258P+A276S+H358D+A364G+S388K+D470T+R483V+L484S;

V45L+A117S+L149R+S214Q+V258P+A276S+H358D+A364G+S388K+D470T+L530R;

V45L+A117S+L149R+S214Q+V258P+A276S+H358D+A364G+S388K+D470T+R483V+L484D;

V45L+A117S+L149R+S214Q+V258P+A276S+H358D+A364G+S388K+D470T+R483V+S528H;

V45L+A117S+L149R+S214Q+V258P+A276S+H358D+A364G+S388K+D470T+R483V+S501N;

V45L+A117S+L149R+S173Y+S214Q+V258P+A276S+H358D+A364G+S388K+D470T+L530R;

V45L+A117S+L149R+S214Q+V258P+A276S+H358D+A364G+S388K+D470T+R483V+A499L;

V45L+S92D+A117S+L149R+S214Q+V258P+A276S+H358D+A364G+S388K+D470T+R483V;

V45L+A117S+L149R+S214Q+V258P+A276S+Q314A+H358D+A364G+S388K+D470T+R483V;

V45L+A79C+L149R+S214Q+V258P+A276S+A364G+S388K;

V45L+A117S+L149R+S214Q+V258P+A276S+N355G+H358D+A364G+S388K+D470T+L530R;

V45L+A117S+L149R+S214Q+V258P+A276S+V350L+H358D+A364G+S388K+D470T+L530R;

V45L+A117S+L149R+S214Q+V258P+A276S+H358D+A364G+S388K+D470T+S481S+L530R;

V45L+A117S+L149R+S214Q+V258P+A276S+H358D+A364G+S388K+D470T+Q496E+L530R;

V45L+A117S+L149R+N168G+S214Q+V258P+A276S+H358D+A364G+S388K+D470T+L530R;

V45L+A117S+L149R+S214Q+V258P+A276S+H358D+A364G+S388K+D470T+V498I+L530R;

V45L+A117S+L149R+S214Q+V258P+A276S+H358D+A364G+S388K+D470A+R483V;

V45N+A117S+L149R+S214Q+V258P+A276S+H358D+A364G+S388K+D470T+R483V;

V45L+A117S+L149R+S214Q+V258P+A276S+H358D+A364G+S388K+D470T+R483V+A499Y;

V45L+A79C+L149R+V258P+A276S+Q314A+A364G+S388K;

V45L+A79C+L149R+V258P+A276S+L281V+A364G+S388K;

V45L+A117S+L149R+S214Q+V258P+A276S+A286S+H358D+A364G+S388K+D470T+L530R;

V45L+A117S+L149R+S214Q+V258P+A276S+H358D+A364G+S388K+D470T+S501N+L530R;

V45L+A117S+L149R+S214Q+V258P+A276S+H358D+A364G+S388K+D470T+N476N+L530R;

V45L+A117S+L149R+S214Q+V258P+A276S+H358D+A364G+S388K+D470T+T477V+L530R;

V45L+A117S+L149R+S214Q+V258P+A276S+H358D+A364G+S388H+D470T+L530R;

V45L+A117S+L149R+S214Q+V258P+A276S+H358D+A364G+S388K+D470T+L478N+L530R;

V45L+S92D+A117S+L149R+S214Q+V258P+A276S+H358D+A364G+S388K+D470T+L530R;

V45L+A117S+L149R+S214Q+V258P+A276S+H358D+A364G+S388K+D470T+V500V+L530R;

V45L+A117S+L149R+S214Q+V258P+A276S+H358D+A364G+S388K+D470T+R483V+V500V;

V45L+A117S+L149R+S214Q+V258P+A276S+H358D+A364G+S388K+D470T+L478P+L530R;

V45L+A117S+L149R+S214Q+V258P+A276S+H358D+A364G+S388K+D470T+R483V+S501E;

V45L+A117S+L149R+S214Q+V258P+A276S+H358D+A364G+S388K+D470T+D525L+L530R;

V45L+A117S+L149R+S214Q+V258P+A276S+H358D+A364G+S388K+D470T+G526T+L530R;

V45L+A117S+L149R+S214Q+V258P+A276S+H358D+A364G+S388K+D470T+N527H+L530R;

V45L+A117S+L149R+S214Q+V258P+A276S+H358D+A364G+S388K+D470T+T477K+L530R;

M28P+V45L+A117S+L149R+S214Q+V258P+A276S+H358D+A364G+S388K+D470T+L530R;

D9E+V45L+A117S+L149R+S214Q+V258P+A276S+H358D+A364G+S388K+D470T+R483V;

V45L+A117S+L149R+S214Q+V258P+A276S+H358D+A364G+S388K+D470T+S501E+L530R;

V45L+A117S+L149R+S214Q+W250Y+V258P+A276S+H358D+A364G+S388K+D470T+L530R;

V45L+A117S+L149R+S214Q+V258P+A276S+H358D+A364G+S388K+D470T+A499L+L530R;

V45L+A117S+L149R+S214Q+V258P+A276S+H358D+A364G+S388K+D470T+A472I+L530R;

V45L+A117S+N127D+L149R+S214Q+V258P+A276S+H358D+A364G+S388K+D470T+R483V;

V45L+A117S+L149R+S214Q+V258P+A276S+H358D+A364G+S388K+D470T+V500T+L530R;

V45L+A117S+L149R+S214Q+V258P+A276S+H358D+A364G+S388K+D470T+R483V+N527H;

V45N+A117S+L149R+S214Q+V258P+A276S+H358D+A364G+S388K+D470T+L530R;

V45L+A117S+L149R+S214Q+V258P+A276S+H358D+A364G+S388K+D470T+T480N+L530R;

V45L+A117S+L149R+S214Q+V258P+A276S+H358D+A364G+S388K+D470T+R483V+V497P;

V45L+A117S+L149R+S214Q+V258P+A276S+H358D+A364G+S388K+D470T+R483V+V498I;

V45L+A79C+L149R+S173Y+V258P+A276S+A364G+S388K;

V45L+A117S+L149R+S214Q+V258P+A276S+H358D+A364G+S388K+D470T+T480N+R483V;

V45L+A117S+L149R+S214Q+H219V+V258P+A276S+H358D+A364G+S388K+D470T+L530R;

V45L+A117S+L149R+S214Q+V258P+A276S+H358D+A364G+S388A+D470T+L530R;

M28P+V45L+A117S+L149R+S214Q+V258P+A276S+H358D+A364G+S388K+D470T+R483V;

V45L+A117S+L149R+A170S+S214Q+V258P+A276S+H358D+A364G+S388K+D470T+R483V;

V45L+A79C+L149R+G187K+V258P+A276S+A364G+S388K;

V45L+A117S+L149R+S214Q+V258P+A276S+H358D+A364G+S388K+S389H+D470T+L530R;

V45L+D113T+A117S+L149R+S214Q+V258P+A276S+H358D+A364G+S388K+D470T+L530R;

V45L+A117S+L149R+N163H+S214Q+V258P+A276S+H358D+A364G+S388K+D470T+R483V;

V45L+A117S+L149R+S214Q+V258P+A276S+H358D+A364G+S388A+D470T+R483V;

V45L+A117S+L149R+S214Q+V258P+A276S+H358D+A364G+S388L+D470T+R483V;

V45L+Q61R+A79C+L149R+V258P+A276S+A364G+S388K;

V45L+A79C+L149R+N168G+V258P+A276S+A364G+S388K;

V45L+A117S+L149R+T211H+S214Q+V258P+A276S+H358D+A364G+S388K+D470T+L530R;

V45L+A79C+L149R+T215V+V258P+A276S+A364G+S388K;

V45L+A117S+L149R+S214Q+V258P+A276S+H358D+A364G+S388K+D470T+R483V+V500T;

V45L+A79C+L149R+V258P+A276S+A286S+A364G+S388K;

V45L+A79C+L149R+H219V+V258P+A276S+A364G+S388K;

V45L+A79C+L149R+A170S+V258P+A276S+A364G+S388K;

V45L+D113T+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A79C+L149R+V258P+A276S+A364G+S388K;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+T215I+V258P+A276S+H358D+A364G+S388K+D470T+R483V;

V45L+A117S+L149R+S214Q+H219I+V258P+A276S+H358D+A364G+S388K+D470T+R483V;

V45L+A117S+L149R+S214Q+V258P+A276S+H358D+A364G+S388K+D470T+L478N;

V45L+A117S+L149R+S214Q+V258P+A276S+L281V+H358D+A364G+S388K+D470T;

V45L+A117S+L149R+S214E+V258P+A276S+H358D+A364G+S388K+D470T;

V45L+T70R+A117S+L149R+S214Q+V258P+A276S+L281V+H358D+A364G+S388K+G394A+C468L+D470T+R483V;

V45L+A117S+L149R+S214Q+V258P+A276S+H358D+A364G+Q385C+S388K+D470T+L530R;

V45L+A117S+L149R+S214Q+V258P+A276S+H358D+A364G+T366I+S388K+D470T;

V45L+A117S+L149R+S214Q+V258P+A276S+H358D+A364G+S388K+D470T+S473G;

V45L+A117S+L149R+S214Q+V258P+A276S+H358D+A364G+S388K+D470T+L478P;

V45L+A117S+L149R+S214Q+V258P+A276S+H358D+A364G+S388K+G394D+D470T;

V45L+A117S+L149R+S214Q+V258P+A276S+A364G+Q384A+S388K+D470T+R483V;

D11E+V45L+A79C+L149R+V258P+A276S+A364G+S388K;

V45L+A117S+L149R+S214Q+V258P+A276S+H358D+A364G+S388K+D470T+R483V+L530R;

V45L+A117S+L149R+S214Q+V258P+A276S+H358D+A364G+S388K+D470T+N529T;

V45L+A117S+L149R+S214Q+V258P+A276S+H358D+A364G+S388K+G394Q+D470T;

V45L+A117S+L149R+G187L+S214Q+V258P+A276S+H358D+A364G+S388K+D470T;

V45L+A79C+L149R+V258P+A276S+A286E+A364G+S388K;

V45L+A117S+L149R+S214Q+V258P+A276S+H358D+A364G+S388K+D470T+R483V+G503L;

V45L+A117S+L149R+S214Q+V258P+A276S+Q314A+H358D+A364G+S388K+D470T;

V45L+A117S+L149R+S214Q+V258P+A276S+V350L+H358D+A364G+S388K+D470T;

V45L+A117S+L149R+G187K+S214Q+V258P+A276S+H358D+A364G+S388K+D470T;

V45L+A117S+L149R+S214Q+W250Y+V258P+A276S+H358D+A364G+S388K+D470T;

V45L+A117S+L149R+S214Q+V258P+A276S+A286E+H358D+A364G+S388K+D470T;

V45L+A117S+L149R+S214Q+V258P+A276S+H358D+A364G+Q384A+S388K+D470T;

V45L+A117S+L149R+S214Q+V258P+A276S+H358D+A364G+S388R+D470T;

V45L+A117S+L149R+S214Q+V258P+A276S+H358D+A364G+S388K+D470T+S528H;

V45L+A117S+L149R+S214Q+T215V+V258P+A276S+H358D+A364G+S388K+D470T+R483V;

V45L+A117S+L149R+S214Q+V258P+A276S+H358D+A364G+S388K+D470T+Q496R;

V45L+A117S+L149R+N163H+S214Q+V258P+A276S+H358D+A364G+S388K+D470T;

V45L+A117S+L149R+S214Q+V258P+A276S+S282N+H358D+A364G+S388K+D470T;

V45L+A117S+L149R+S214Q+V258P+A276Q+H358D+A364G+S388K+D470T;

D11E+V45L+A117S+L149R+S214Q+V258P+A276S+H358D+A364G+S388K+D470T;

V45L+A117S+L149R+S214Q+V258P+A276S+H358D+A364G+T366V+S388K+D470T;

V45L+A117S+L149R+S214Q+V258P+A276S+H358D+A364G+S388K+D470T+S473K;

V45L+A79C+L149R+T180Q+V258P+A276S+A364G+S388K;

V45L+A117S+L149R+S214Q+V258P+A276S+H358D+A364G+S388K+D470T+S481A+R483V;

V45L+A117S+L149R+S214Q+V258P+A276S+H358D+A364G+S388R+D470T+R483V;

V45L+A117S+L149R+S214Q+V258P+A276S+Q314A+A364G+S388K+D470T+R483V;

V45L+S92D+A117S+L149R+S214Q+V258P+A276S+H358D+A364G+S388K+D470T;

V45L+A117S+L149R+S214Q+V258P+N260H+A276S+H358D+A364G+S388K+D470T;

V45L+A117S+L149R+G187S+S214Q+V258P+A276S+H358D+A364G+S388K+D470T;

V45L+A117S+L149R+S214Q+V258P+A276S+H358D+A364G+S388K+D470T+T477K;

V45L+A117S+L149R+S214Q+V258P+A276S+H358D+A364G+S388K+D470T+N474G;

V45L+A117S+L149R+S214Q+V258P+A276S+H358D+A364G+S388K+N395D+S501N V45L+A117S+N127D+L149R+S214Q+V258P+A276S+H358D+A364G+S388K+D470T;

V45L+A117S+L149R+S214Q+V258P+A276S+H358D+A364G+S388K+V393R+D470T;

V45L+A117S+L149R+S173Y+S214Q+V258P+A276S+H358D+A364G+S388K+D470T;

V45L+A117S+L149R+S214Q+V258P+A276S+H358D+A364G+S388H+D470T;

V45L+A117S+L149R+S214Q+T215V+V258P+A276S+H358D+A364G+S388K+D470T+L530R;

V45L+A117S+L149R+G187S+S214Q+V258P+A276S+H358D+A364G+S388K+D470T+L530R;

V45L+A117S+L149R+T211H+S214Q+V258P+A276S+H358D+A364G+S388K+D470T+R483V;

V45L+A117S+L149R+S214Q+V258P+A276S+H358D+A364G+S388K+D470T+T480N;

V45L+A117S+L149R+N168G+S214Q+V258P+A276S+H358D+A364G+S388K+D470T;

V45L+A79C+L149R+H219I+V258P+A276S+A364G+S388K;

V45L+L149R+S214E+V258P+A276S+H358D+A364G+D470T;

V45L+Q61K+A117S+L149R+S214Q+V258P+A276S+A364G+S388K+D470T+R483V;

V45L+D113T+A117S+L149R+S214Q+V258P+A276S+H358D+A364G+S388K+D470T;

V45L+A117S+L149R+S214Q+V258P+A276S+A364G+T366V+S388K+D470T+R483V;

V45L+A117S+L149R+S214Q+V258P+A276S+H358T+A364G+S388K+D470T+R483V;

V45L+A117S+L149R+S214Q+V258P+A276S+A364G+S388K+V393R+D470T+R483V;

V45N+A79C+L149R+V258P+A276S+A364G+S388K;

V45L+A117S+L149R+S214Q+V258P+A276S+H358D+A364G+S388K+D470T+Q496Q;

V45L+A117S+L149R+S214Q+V258P+A276S+A364G+S388K+G394Q+D470T+R483V;

V45L+A79C+L149R+V258P+A276S+V350L+A364G+S388K;

V45L+L149R+S214E+V258P+A276S+H358D+A364G+D470T;

V45L+A117S+L149R+S214Q+V258P+A276S+A364G+T366I+S388K+D470T+R483V;

V45L+A117S+L149R+S214Q+V258P+A276S+H358D+A364G+S388K+D470T+R483V+G526T;

V45L+A117S+L149R+S214Q+V258P+A276S+H358D+A364G+S388K+D470T+L484S;

V45L+A79C+A117S+L149R+S214Q+V258P+A276S+H358D+A364G+S388K;

V45L+A117S+L149R+S214Q+V258P+A276S+H358D+A364G+S388K+D470T+A499L;

V45L+A117S+L149R+S214Q+V258P+A276S+H358T+A364G+S388K+D470T+L530R;

V45L+A79C+D113T+L149R+V258P+A276S+A364G+S388K;

V45L+A117S+L149R+S214Q+V258P+A276S+H358D+A364G+S388K+D470T+R483V+D525L;

V45L+A117S+L149R+S214Q+V258P+A276S+H358D+A364G+S388K+D470T+V498I;

V45L+A117S+L149R+S214Q+V258P+A276S+H358D+A364G+S388L+D470T;

V45L+T70R+A117S+L149R+S214Q+V258P+A276S+A364G+S388K+D470T+R483V;

V45L+A117S+L149R+S214Q+V258P+A276S+H358D+A364G+S388K+D470T+S528H+L530R;

V45L+A117S+L149R+S214Q+V258P+A276S+A364G+S388K+D470T+N476D+R483V;

D9E+V45L+A117S+L149R+S214Q+V258P+A276S+A364G+S388K+D470T+R483V;

V45L+A117S+L149R+S214Q+V258P+A276S+H358D+A364G+S388K+D470T+S501E;

V45L+A117S+L149R+S214Q+V258P+A276S+H358D+A364G+S388K+D470T+S481A;

V45L+A117S+L149R+S214Q+H219V+V258P+A276S+H358D+A364G+S388K+D470T;

V45L+A117S+L149R+S214Q+V258P+A276S+H358D+A364G+S388K+D470T+S481S;

V45L+A117S+L149R+G187K+S214Q+V258P+A276S+A364G+S388K+D470T+V45L+A117S+L149R+S214Q+V258P+A276S+A364G+S388K+N395D+D470T+R483V;

V45L+A117S+L149R+S214Q+V258P+A276S+H358D+A364G+S388K+I469T+D470T+R483V;

V45L+A79C+N127D+L149R+V258P+A276S+A364G+S388K;

D9E+V45L+A79C+L149R+V258P+A276S+A364G+S388K;

V45L+L149R+S214E+V258P+A276S+H358D+A364G+D470T;

V45L+A117S+L149R+G187L+S214Q+V258P+A276S+A364G+S388K+D470T+R483V;

V45L+A117S+L149R+S214Q+V258P+A276S+H358D+A364G+S388K+D470T+N529T+L530R;

V45L+A79C+L149R+W250Y+V258P+A276S+A364G+S388K;

V45L+A117S+L149R+S214Q+V258P+A276S+H358D+A364G+S388K+D470T+V497P;

V45L+A117S+L149R+S214Q+V258P+A276S+H358D+A364G+S388K+D470T+N527H;

V45L+A117S+L149R+S214Q+V258P+A276S+A364G+S388K+D470T+R483V+Q496Q;

V45L+A117S+L149R+N168G+S214Q+V258P+A276S+A364G+S388K+D470T+R483V;

V45L+A117S+L149R+S214Q+V258P+A276S+A364G+S388K+D470T+N476N+R483V;

V45L+A117S+L149R+S214Q+V258P+A276S+A364G+S388K+D470T+T477V+R483V;

V45L+A117S+L149R+S214Q+V258P+A276S+V350L+A364G+S388K+D470T+R483V;

V45L+A117S+L149R+S214Q+V258P+A276S+A364G+S388K+D470T+L478N+R483V;

V45L+A117S+L149R+S214E+V258P+A276S+A364G+S388K+D470T+R483V;

M28P+V45L+A117S+L149R+S214Q+V258P+A276S+H358D+A364G+S388K+D470T;

V45L+A117S+L149R+S214Q+V258P+A276S+A364G+S388K+V393H+D470T+R483V;

V45L+Q61R+A117S+L149R+S214Q+V258P+A276S+A364G+S388K+D470T+R483V;

V45L+A117S+L149R+S214Q+V258P+A276S+A364G+S388K+D470T+R483V+V497P;

V45L+A117S+L149R+S214Q+V258P+A276S+A364G+S388K+D470T+R483V+V498I;

V45L+A117S+L149R+S214Q+V258P+A276S+S282N+H358D+A364G+S388K+D470T+L530R;

V45L+T70R+A117S+L149R+S214Q+V258P+A276S+L281V+H358D+A364G+C375V+S388K+G394A+D470T+R483V;

V45L+A117S+L149R+S214Q+V258P+A276S+A286E+A364G+S388K+D470T+R483V;

V45L+A117S+L149R+S214Q+V258P+A276S+A364G+S388R+D470T+R483V;

V45L+A117S+L149R+S214Q+V258P+A276S+A364G+S388K+D470A+R483V;

V45L+A117S+L149R+S214Q+V258P+A276S+A364G+S388K+D470T+T477K+R483V;

V45L+A117S+L149R+S214Q+V258P+A276S+A364G+S388K+D470T+R483V+Q496E;

V45L+A117S+L149R+S214Q+V258P+A276S+A364G+S388K+D470T+R483V+Q496R;

V45L+A117S+L149R+S214Q+V258P+A276S+A364G+S388K+D470T+R483V+A499L;

V45L+L149R+S214E+V258P+A276S+H358D+A364G+D470T;

V45L+L149R+S214E+V258P+A276S+H358D+A364G+D470T;

V45L+A117S+L149R+S214Q+T215I+V258P+A276S+H358D+A364G+S388K+D470T;

V45L+T70R+A117S+L149R+S214Q+V258P+A276S+L281V+H358D+A364G+S388K+G394A+C468S+D470T+R483V;

V45L+A117S+L149R+S214Q+V258P+A276S+A364G+S388K+Y391V+D470T+R483V;

V45L+A117S+L149R+S214Q+V258P+A276Q+A364G+S388K+D470T+R483V;

V45L+A117S+L149R+S214Q+V258P+A276S+H358D+A364G+S388K+D470T+G503L+L530R;

V45L+A117S+L149R+S214Q+H219I+V258P+A276S+H358D+A364G+S388K+D470T+L530R;

V45L+A117S+L149R+S214Q+V258P+A276S+H358D+A364G+S388K+D470T+N476D;

V45L+A117S+L149R+S214Q+V258P+A276S+H358D+A364G+S388K+V393H+D470T;

V45L+A117S+L149R+S214Q+V258P+A276S+A364G+S388K+D470T+R483V;

V45L+A117S+L149R+S214Q+V258P+A276S+A286S+A364G+S388K+D470T+R483V;

V45L+A117S+L149R+S214Q+V258P+A276S+A364G+S388K+D470T+R483V+S501E;

V45L+A117S+L149R+S214Q+V258P+A276S+A364G+S388K+D470T+R483V+N529T;

V45L+A117S+L149R+S214Q+V258P+A276S+A364G+S388K+D470T+R483V+S501N, V45L+A117S+L149R+S214Q+V258P+A276S+H358D+A364G+S388K+D470T;

V45L+A117S+L149R+S214Q+V258P+A276S+A364G+S388K+D470T+S481S+R483V;

V45L+A117S+L149R+S214Q+V258P+A276S+A364G+S388K+D470T+L478P+R483V;

D11E+V45L+A117S+L149R+S214Q+V258P+A276S+A364G+S388K+D470T+R483V;

V45L+A117S+L149R+S214Q+V258P+A276S+H358D+A364G+S388K+D470T+T477V;

V45L+A117S+L149R+S214Q+V258P+A276S+H358D+A364G+S388K+D470T+V497P+L530R;

V45L+A117S+L149R+T211H+S214Q+V258P+A276S+H358D+A364G+S388K+D470T;

V45L+A117S+L149R+S214Y+V258P+A276S+H358D+A364G+S388K+D470T+R483V;

V45L+A117S+L149R+S214Q+W250Y+V258P+A276S+A364G+S388K+D470T+R483V;

V45L+S92D+A117S+L149R+S214Q+V258P+A276S+A364G+S388K+D470T+R483V;

V45L+A117S+L149R+S214Q+V258P+A276S+A364G+S388K+D470T+R483V+N527H;

V45N+A117S+L149R+S214Q+V258P+A276S+A364G+S388K+D470T+R483V;

V45L+A117S+L149R+S214Q+V258P+A276S+H358D+A364G+Q385C+S388K+D470T;

V45L+A117S+L149R+S173Y+S214Q+V258P+A276S+A364G+S388K+D470T+R483V;

V45L+A117S+L149R+S214Q+V258P+A276S+A364G+S388H+D470T+R483V;

V45L+A117S+L149R+S214Q+V258P+A276S+A364G+S388K+S389H+D470T+V45L+A117S+L149R+S214Q+H219V+V258P+A276S+A364G+S388K+D470T+R483V;

V45L+A79C+A117S+L149R+S214Q+V258P+A276S+A364G+S388K+D470T;

V45L+A117S+L149R+S214Q+T215I+V258P+A276S+H358D+A364G+S388K+D470T+L530R;

V45L+A117S+L149R+S214Q+V258P+A276S+H358D+A364G+S388K+D470T+Q496E;

V45L+A117S+L149R+A170S+S214Q+V258P+A276S+H358D+A364G+S388K+D470T;

V45L+A117S+L149R+S214Q+V258P+A276S+H358D+A364G+S388K+S389V+D470T+R483V;

V45L+A117S+L149R+S214Q+V258P+A276S+H358D+A364G+S388K+S389V+D470T+L530R;

V45L+A117S+L149R+S214Q+V258P+A276S+H358D+A364G+S388A+D470T;

V45L+A117S+L149R+S214Q+V258P+A276S+H358D+A364G+S388K+S389V+D470T;

V45L+T70R+A117S+L149R+S214Q+V258P+A276S+H358D+A364G+S388K+D470T;

V45L+A117S+L149R+S214Q+H219I+V258P+A276S+A364G+S388K+D470T+R483V;

V45L+A79C+L149R+N163H+V258P+A276S+A364G+S388K;

V45L+A117S+L149R+S214Q+V258P+A276S+A364G+S388K+D470T+R483V+A499Y;

V45L+A117S+L149R+S214Q+V258P+A276S+L281V+A364G+S388K+D470T+R483V;

V45L+A117S+L149R+S214Q+V258P+A276S+A364G+S388K+D470T+R483V+V500T;

V45L+A117S+L149R+T180Q+S214Q+V258P+A276S+H358D+A364G+S388K+D470T;

V45L+A117S+L149R+S214Q+V258P+A276S+H358D+A364G+S388K+D470T+V500V;

V45L+A117S+L149R+S214Q+V258P+A276S+H358D+A364G+S388K+D470T+G526T;

V45L+C60L+T70R+A117S+L149R+S214Q+V258P+A276S+L281V+H358D+A364G+S388K+G394A+D470T+R483V;

V45L+C69L+T70R+A117S+L149R+S214Q+V258P+A276S+L281V+H358D+A364G+S388K+G394A+D470T+R483V;

V45L+A117S+L149R+S214Q+V258P+A276S+A364G+S388A+D470T+R483V;

V45L+A117S+L149R+S214Q+V258P+A276S+A286S+H358D+A364G+S388K+D470T;

V45L+A117S+L149R+S214Q+V258P+A276S+H358D+A364G+S388K+D470T+R483V+V497D;

V45L+A117S+L149R+S214Q+V258P+A276S+H358D+A364G+S388K+D470A+L530R;

V45L+A117S+L149R+S214Q+V258P+A276S+A364G+S388K+D470T+R483V+D525L;

V45L+A117S+L149R+S214Q+V258P+A276S+H358D+A364G+S388K+D470T+N476N;

V45L+A117S+L149R+S214Q+V258P+A276S+L281V+A364G+S388K+D470T;

V45L+A117S+L149R+S214Q+V258P+A276S+A364G+Q384A+S388K+D470T;

V45L+A117S+L149R+S214Q+V258P+A276S+A286E+A364G+S388K+D470T;

V45L+A117S+L149R+S214Q+V258P+A276S+A364G+S388K+D470T+T477K;

V45L+A117S+L149R+S214Q+V258P+A276S+H358D+A364G+S388K+S389H+D470T;

V45L+A117S+L149R+S214Y+V258P+A276S+H358D+A364G+S388K+D470T+L530R;

V45L+A117S+L149R+S214Y+V258P+A276S+H358D+A364G+S388K+D470T;

V45L+A117S+L149R+S214Q+V258P+A276S+A364G+S388K+G394A+D470T;

V45L+A117S+L149R+S214Q+V258P+A276S+A364G+S388K+D470T+N476D;

V45L+A117S+L149R+S214Q+V258P+A276S+H358D+A364G+S388K+D470T+L478N+R483V;

V45L+C60A+T70R+A117S+L149R+S214Q+V258P+A276S+L281V+H358D+A364G+S388K+G394A+D470T+R483V;

V45L+A117S+L149R+S214Q+V258P+A276S+A364G+S388K+V393H+D470T;

V45L+A117S+L149R+S214Q+V258P+A276S+A364G+S388K+N395D+D470T;

A25W+V45L+A117S+L149R+S214Q+V258P+A276S+H358D+A364G+S388K+D470T;

V45L+T70R+A117S+L149R+S214Q+C243L+V258P+A276S+L281V+H358D+A364G+S388K+G394A+D470T+R483V;

V45L+A117S+L149R+S214Q+V258P+A276S+H358T+A364G+S388K+D470T;

V45L+A117S+L149R+S214Q+V258P+A276S+A364G+S388K+D470T+L478N;

V45L+C60S+T70R+A117S+L149R+S214Q+V258P+A276S+L281V+H358D+A364G+S388K+G394A+D470T+R483V;

V45L+A117S+L149R+S214Q+W250Y+V258P+A276S+A364G+S388K+D470T;

V45L+A117S+L149R+S214Q+T215V+V258P+A276S+A364G+S388K+D470T+R483V;

V45L+Q61R+A117S+L149R+S214Q+V258P+A276S+H358D+A364G+S388K+D470T;

V45L+A117S+L149R+G187L+S214Q+V258P+A276S+A364G+S388K+D470T;

D9E+V45L+A117S+L149R+S214Q+V258P+A276S+A364G+S388K+D470T;

V45L+A117S+L149R+S214Q+V258P+A276S+H358D+A364G;

V45L+C69A+T70R+A117S+L149R+S214Q+V258P+A276S+L281V+H358D+A364G+S388K+G394A+D470T+R483V;

V45L+T70R+A117S+L149R+C161S+S214Q+V258P+A276S+L281V+H358D+A364G+S388K+G394A+D470T+R483V;

V45L+A117S+L149R+S214Q+H219I+V258P+A276S+H358D+A364G+S388K+D470T;

V45L+A117S+L149R+S214Q+V258P+A276S+S282N+A364G+S388K+D470T;

V45L+A117S+L149R+T211H+S214Q+V258P+A276S+A364G+S388K+D470T+R483V;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

D11E+V45L+A117S+L149R+S214Q+V258P+A276S+A364G+S388K+D470T;

V45L+A117S+L149R+S214Q+V258P+A276S+A364G+S388K+D470T+N476N;

V45L+A117S+L149R+S214Q+V258P+A276S+A364G+S388K+G394D+D470T;

V45L+Q61K+A117S+L149R+S214Q+V258P+A276S+A364G+S388K+D470T;

V45L+A117S+L149R+S214Q+V258P+A276S+H358D+A364G+S388K+D470T+G503L;

V45L+A117S+L149R+S214Q+V258P+A276S+A286S+A364G+S388K+D470T;

V45L+A117S+L149R+S214Q+V258P+A276S+H358D+A364G+S388K+D470A;

V45L+A117S+L149R+S214Q+V258P+A276S+A364G+S388K+G394Q+D470T;

V45L+A117S+L149R+S214Q+V258P+A276S+A364G+S388R+D470T;

V45L+A117S+L149R+S214Q+V258P+A276S+A364G+S388K+D470T+L478P;

V45L+A117S+L149R+S214Q+V258P+A276S+V350L+A364G+S388K+D470T;

V45L+D113T+A117S+L149R+S214Q+V258P+A276S+A364G+S388K+D470T;

V45L+A117S+L149R+T180Q+S214Q+V258P+A276S+A364G+S388K+D470T;

V45L+C69V+T70R+A117S+L149R+S214Q+V258P+A276S+L281V+H358D+A364G+S388K+G394A+D470T+R483V;

V45L+A117S+L149R+S214Q+V258P+A276S+H358D+A364G+S388K+D470T+D525L;

V45L+A117S+L149R+S214Q+V258P+A276S+A364G+S388K+D470T;

V45L+A117S+L149R+S214Q+V258P+A276S+H358D+A364G+S388K+D470T+V500T;

V45L+A117S+L149R+S214Q+V258P+A276S+A364G+S388K+S389H+D470T;

V45L+A117S+L149R+S214Q+V258P+A276S+A364G+S388K+V393R+D470T;

V45L+A117S+L149R+S214Q+V258P+A276S+A364G+S388K+D470T+Q496R;

V45L+A117S+N127D+L149R+S214Q+V258P+A276S+A364G+S388K+D470T;

V45L+A117S+L149R+S214Q+V258P+A276S+A364G+S388K+D470T+A499Y;

V45L+A117S+L149R+S214Q+T215V+V258P+A276S+H358D+A364G+S388K+D470T;

V45L+A117S+L149R+S214Q+V258P+A276S+H358D+A364G+S388K+D470T+T480L;

V45L+A117S+L149R+S214Q+V258P+A276S+A364G+S388L+D470T;

V45L+A117S+L149R+S173Y+S214Q+V258P+A276S+A364G+S388K+D470T;

V45L+S92D+A117S+L149R+S214Q+V258P+A276S+A364G+S388K+D470T;

V45L+A117S+L149R+S214Q+V258P+A276S+A364G+S388K+S389V+D470T+R483V;

V45L+A117S+L149R+S214Q+V258P+A276S+A364G+S388K+D470T+N529T;

V45L+C60V+T70R+A117S+L149R+S214Q+V258P+A276S+L281V+H358D+A364G+S388K+G394A+D470T+R483V;

V45L+A117S+L149R+S214Q+V258P+A276S+A364G+S388K+D470T+R483V+V497E;

V45L+A117S+L149R+S214Q+V258P+A276S+H358D+A364G+S388K+L478N;

V45L+T70R+A117S+L149R+S214Q+V258P+A276S+A364G+S388K+D470T;

M28P+V45L+A117S+L149R+S214Q+V258P+A276S+A364G+S388K+D470T;

V45L+A117S+L149R+S214Q+V258P+A276Q+A364G+S388K+D470T;

V45L+A117S+L149R+S214Q+V258P+A276S+H358D+A364G+S388K+D470V+R483V;

V45L+A117S+L149R+S214Y+V258P+A276S+A364G+S388K+D470T+R483V;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

V45L+Q61R+A117S+L149R+S214Q+V258P+A276S+A364G+S388K+D470T;

V45L+A117S+L149R+S214Q+T215I+V258P+A276S+A364G+S388K+D470T;

V45L+A117S+L149R+S214Q+V258P+A276S+A364G+S388K+D470T+Q496E;

V45L+A117S+L149R+G187K+S214Q+V258P+A276S+A364G+S388K+D470T;

V45L+A117S+L149R+S214Q+V258P+A276S+A364G+S388K+D470T+W495I;

V45L+A117S+L149R+S214Q+V258P+A276S+A364G+S388K+D470T+V498I;

V45L+A117S+L149R+S214Q+V258P+A276S+H358D+A364G+S388K+D470K+R483V;

V45L+A117S+L149R+S214Q+V258P+A276S+A364G+S388K+D470T+V500V;

D11E+V45L+A117S+L149R+S214Q+V258P+A276S+H358D+A364G+S388K;

V45L+A117S+L149R+A170S+S214Q+V258P+A276S+A364G+S388K+D470T;

V45L+A117S+L149R+S214Q+V258P+A276S+A364G+S388K+Y391V+D470T;

V45L+A117S+L149R+S214Q+V258P+A276S+Q314A+A364G+S388K+D470T;

V45L+A117S+L149R+S214Q+V258P+A276S+A364G+S388K+D470T+T477V;

A25W+V45L+A79C+L149R+S214E+V258P+A276S+A364G;

V45L+A117S+L149R+S214Q+V258P+A276S+H358D+A364G+S388K+G394Q;

V45L+A117S+L149R+S214Q+H219V+V258P+A276S+A364G+S388K+D470T;

V45L+A117S+L149R+S214Q+V258P+A276S+A364G+S388K+D470T+Q496Q;

V45L+T70R+A117S+L149R+S214Q+V258P+A276S+L281V+H358D+A364G+C375A+S388K+G394A+D470T+R483V;

V45L+A117S+L149R+D192E+S214Q+V258P+A276S+H358D+A364G;

V45L+A117S+L149R+S214Q+V258P+A276S+H358D+A364G+S388K+D470T+A472L;

V45L+A117S+L149R+S214Q+V258P+A276S+H358D+A364G+S388K+A499Y;

V45L+A117S+L149R+S214Q+T215V+V258P+A276S+A364G+S388K+D470T;

V45L+A117S+L149R+S214Q+V258P+A276S+A364G+Q385C+S388K+D470T;

V45L+A117S+L149R+S214Q+V258P+A276S+A364G+S388A+D470T;

V45L+A117S+L149R+S214Q+V258P+A276S+A364G+S388H+D470T;

V45L+A117S+L149R+S214Q+V258P+A276S+A364G+S388K+D470T+T480N;

V45L+A117S+L149R+S214Q+V258P+A276S+A364G+S388K+D470T+V497P;

V45L+A117S+L149R+S214Q+V258P+A276S+A364G+S388K+D470T+A499L;

V45L+A117S+L149R+S214Q+V258P+A276S+A364G+S388K+D470T+S501N;

V45L+A117S+L149R+S214Q+V258P+A276S+A364G+D470T;

V45L+A117S+L149R+S214Q+V258P+A276S+H358D+A364G+Q384A+S388K;

V45L+T70R+A117S+L149R+S214Q+V258P+A276S+L281V+H358D+A364G+C375S+S388K+G394A+D470T+R483V;

V45L+A117S+L149R+S214Q+V258P+A276S+A364G+S388L+D470T+R483V;

V45L+A79C+S92D+L149R+V258P+A276S+A364G+S388K;

V45L+T70R+A117S+L149R+S214Q+V258P+A276S+L281V+H358D+A364G+S388K+G394A+D470T+R483V+C540A;

V45N+A117S+L149R+S214Q+V258P+A276S+A364G+S388K+D470T;

V45L+A117S+L149R+S214Q+V258P+A276S+H358D+A364G+S388K+G394A;

V45L+C69S+T70R+A117S+L149R+S214Q+V258P+A276S+L281V+H358D+A364G+S388K+G394A+D470T+R483V;

V45L+A117S+L149R+S214Q+V258P+A276S+H358D+A364G+S388K+V498I;

V45L+A117S+L149R+S214Q+V258P+A276S+H358D+A364G;

V45L+A117S+L149R+S214Q+H219I+V258P+A276S+A364G+S388K+D470T;

V45L+A117S+L149R+N163H+S214Q+V258P+A276S+A364G+S388K+D470T;

V45L+L149R+S214Q+V258P+A276S+H358D+A364G;

V45L+A117S+L149R+S214Q+V258P+A276S+Q314A+H358D+A364G+S388K;

V45L+A117S+L149R+S173Y+S214Q+V258P+A276S+H358D+A364G+S388K;

V45L+A117S+L149R+S214E+V258P+A276S+H358D+A364G+S388K;

V45L+A117S+L149R+S214Q+V258P+A276S+H358D+A364G+S388K+G394D;

A25W+V45L+A79C+L149R+V258P+A276S+A364G;

V45L+L149R+S214Q+V258P+A276S+A286E+A364G+S388K;

V45L+Q61R+A117S+L149R+S214Q+V258P+A276S+H358D+A364G+S388K;

V45L+A117S+L149R+S214Q+V258P+A276S+H358D+A364G+T366V+S388K;

V45L+A117S+L149R+D192E+S214Q+V258P+A276S+H358D+A364G;

V45L+A117S+L149R+T180Q+S214Q+V258P+A276S+H358D+A364G+S388K;

V45L+A117S+L149R+S214Q+T215I+V258P+A276S+H358D+A364G+S388K;

V45L+A117S+L149R+S214Q+W250Y+V258P+A276S+H358D+A364G+S388K;

V45L+L149R+S214Q+T215I+V258P+A276S+A364G+S388K;

V45L+A117S+L149R+S214Q+V258P+A276S+H358D+A364G+S388K+N476D;

V45L+D113T+A117S+L149R+S214Q+V258P+A276S+A364G+S388K+D470T+R483V;

V45L+A117S+L149R+S214Q+T215K+V258P+A276S+A364G+S388K+D470T+R483V;

V45L+A117S+L149R+S214Q+V258P+A276S+H358D+A364G+S388K;

V45L+A117S+L149R+S214Q+V258P+A276S+H358D+A364G+S388K+T477V;

V45L+A117S+L149R+S214Q+V258P+A276S+H358D+A364G+S388K+L478P;

V45L+A117S+L149R+S214Q+V258P+A276S+H358D+A364G+S388K+S481A;

A25W+V45L+L149R+S214Q+V258P+A276S+A364G+S388K;

V45L+A117S+L149R+T211H+S214Q+V258P+A276S+A364G+S388K+D470T;

V45L+A117S+L149R+S214Q+T215K+V258P+A276S+H358D+A364G+S388K+D470T+R483V;

V45L+A79C+L149R+V258P+A276S+L281V+A364G;

V45L+A117S+L149R+S214Q+V258P+A276S+A364G+S388K+R483V;

V45L+A117S+L149R+S214Q+V258P+A276S+H358D+A364G+S388K+V500T;

V45L+A117S+L149R+S214Q+V258P+A276S+V350L+H358D+A364G+S388K;

V45L+A117S+L149R+G187K+S214Q+V258P+A276S+H358D+A364G+S388K;

V45L+T70R+A117S+L149R+S214Q+V258P+A276S+L281V+H358D+A364G+C375L+S388K+G394A+D470T+R483V;

V45L+L149R+A170S+S214Q+V258P+A276S+A364G+S388K;

V45L+A117S+L149R+S214E+V258P+A276S+A364G+S388K+D470T;

V45L+A117S+L149R+S214Q+V258P+A276S+A364G+S388K+D470V+R483V;

V45L+A117S+L149R+S214Q+V258P+A276S+H358D+A364G+S388K+V500V;

V45L+Q61K+L149R+S214Q+V258P+A276S+A364G+S388K;

V45L+L149R+N168G+S214Q+V258P+A276S+A364G+S388K;

V45L+A117S+L149R+N163H+S214Q+V258P+A276S+H358D+A364G+S388K;

V45L+A79C+D95D+L149R+V258P+A276S+A364G+S388K;

V45L+Q77E+A79C+L149R+V258P+A276S+A364G+S388K;

V45L+A117S+L149R+S214Q+V258P+A276S+A364G+S388K+D470T+R483V+V497W;

V45L+L149R+S214Q+V258P+A276S+S282N+A364G+S388K;

V45L+A117S+L149R+S214Q+V258P+A276S+H358D+A364G+S388R;

V45L+A117S+L149R+S214Q+V258P+A276S+H358D+A364G+S388K+S481S;

V45L+A117S+L149R+S214Q+V258P+A276S+A364G+S388K+D470T+V500T;

V45L+T70R+A117S+L149R+S214Q+V258P+A276S+L281V+H358D+A364G+S388K+G394A+D470T+R483V+C540L;

V45L+T70R+A117S+L149R+S214Q+V258P+A276S+L281V+H358D+A364G+S388K+G394A+D470T+R483V+C522S;

V45L+A117S+L149R+S214Q+V258P+A276S+H358D+A364G+S388K+W495S;

V45L+L149R+S214Q+V258P+A276S+Q314A+A364G+S388K;

V45L+D113T+A117S+L149R+S214Q+V258P+A276S+H358D+A364G+S388K;

V45L+A117S+L149R+S214Q+V258P+A276S+H358D+A364G+S388K+D470T+R483V+V497W;

V45L+T70R+A117S+L149R+S214Q+V258P+A276S+L281V+H358D+A364G+S388K+G394A+D470T+R483V+C540S;

V45L+T70R+A117S+L149R+S214Q+V258P+A276S+L281V+H358D+A364G+S388K+G394A+C396S+D470T+R483V;

V45L+T70R+L149R+S214Q+V258P+A276S+A364G+S388K;

V45L+A117S+L149R+S214Q+V258P+A276S+H358D+A364G+S388K+N474G;

V45L+A117S+L149R+S214Q+V258P+A276S+S282N+H358D+A364G+S388K;

V45L+A117S+L149R+S214Q+V258P+A276S+H358D+A364G+S388K+Q496Q;

V45L+A117S+L149R+S214Q+V258P+A276S+A364G+S388K+S389V+D470T;

V45L+T70R+A117S+L149R+S214Q+V258P+A276S+L281V+H358D+A364G+S388K+G394A+C396A+D470T+R483V;

V45L+A117S+L149R+S214Q+V258P+A276S+H358D+A364G+S388K+S501E;

A25W+V45L+L149R+S214E+V258P+A276S+A364G;

V45L+L149R+S214Q+W250Y+V258P+A276S+A364G+S388K;

V45L+L149R+G187K+S214Q+V258P+A276S+A364G+S388K;

V45L+A117S+L149R+S214Q+V258P+A276S+H358D+A364G+S388K+N476N;

V45L+A117S+L149R+S214Q+V258P+A276S+H358D+A364G+S388K+S473K;

V45L+A117S+L149R+G187L+S214Q+V258P+A276S+H358D+A364G+S388K;

C40L+V45L+T70R+A117S+L149R+S214Q+V258P+A276S+L281V+H358D+A364G+S388K+G394A+D470T+R483V;

V45L+A117S+L149R+S214Q+V258P+A276S+H358D+A364G+S388K+Q496E;

A25W+V45L+A117S+L149R+S214Q+V258P+A276S+H358D+A364G+S388K;

V45L+L149R+V258P+A276S+L281V+H358T+A364G+S388K;

V45L+A117S+L149R+S214Q+V258P+A276S+H358D+A364G+S388A;

V45L+A117S+L149R+N168G+S214Q+V258P+A276S+H358D+A364G+S388K;

V45L+A117S+L149R+S214Q+V258P+A276S+H358D+A364G+S388K+S389H;

M28P+V45L+L149R+S214Q+V258P+A276S+A364G+S388K;

V45L+A117S+L149R+S214Q+V258P+A276S+A364G+S388K+D470T+T480L;

V45L+A117S+L149R+S214Q+V258P+A276S+A364G+Q384A+S388K;

V45L+A93S+L149R+A252S+H334D;

C40A+V45L+T70R+A117S+L149R+S214Q+V258P+A276S+L281V+H358D+A364G+S388K+G394A+D470T+R483V;

V45L+L149R+G187L+S214Q+V258P+A276S+A364G+S388K;

V45L+A117S+L149R+S214Q+V258P+A276S+H358D+A364G+Q385C+S388K;

V45L+L149R+S214Q+V258P+A276S+A286S+A364G+S388K;

V45L+S92D+A117S+L149R+S214Q+V258P+A276S+H358D+A364G+S388K;

V45L+A117S+L149R+S214Q+V258P+A276S+H358D+A364G+S388L;

V45L+A117S+L149R+S214Q+V258P+A276S+A286E+A364G+S388K;

D9E+V45L+L149R+S214Q+V258P+A276S+A364G+S388K;

V45L+L149R+S173Y+S214Q+V258P+A276S+A364G+S388K;

V45L+A117S+L149R+A170S+S214Q+V258P+A276S+H358D+A364G+S388K;

V45L+A117S+L149R+S214Q+V258P+A276S+H358D+A364G+S388K+Y391V;

V45L+A117S+L149R+S214Q+V258P+A276S+L281V+A364G+S388K;

V45L+A117S+L149R+S214Q+V258P+A276S+A364G+S388K+G394D;

V45L+A117S+L149R+S214Q+V258P+A276S+A364G+S388K+S473G;

V45L+A79C+L149R+V258P+A276S+H358T+A364G+S388K;

V45L+L149R+S214Q+V258P+A276Q+A364G+S388K;

A25W+V45L+L149R+S214E+V258P+A276S+H358T+A364G;

V45L+A117S+L149R+S214Q+V258P+A276S+H358D+A364G+S388K+T480N;

D9E+V45L+A117S+L149R+S214Q+V258P+A276S+H358D+A364G+S388K;

V45L+A117S+L149R+S214Q+H219I+V258P+A276S+H358D+A364G+S388K;

C40S+V45L+T70R+A117S+L149R+S214Q+V258P+A276S+L281V+H358D+A364G+S388K+G394A+D470T+R483V;

C40V+V45L+T70R+A117S+L149R+S214Q+V258P+A276S+L281V+H358D+A364G+S388K+G394A+D470T+R483V;

A25W+V45L+L149R+S214E+V258P+A276S+A286E+A364G;

V45L+A117S+L149R+S214Q+V258P+A276S+A364G+S388K+G394A;

V45L+A117S+L149R+S214Q+V258P+A276S+H358D+A364G+S388K+A499L;

A25W+V45L+Q61K+L149R+S214E+V258P+A276S+A364G;

V45L+A117S+L149R+S214Q+T215V+V258P+A276S+H358D+A364G+S388K;

A25W+V45L+L149R+V258P+A276S+H358T+A364G+S388K;

V45L+A117S+L149R+S214Q+V258P+A276S+A364G+S388K+D470T+R483V+W495E;

A25W+V45L+A79C+L149R+V258P+A276S;

V45L+L149R+S214Q+V258P+A276S+V350L+A364G+S388K;

D11E+V45L+L149R+S214Q+V258P+A276S+A364G+S388K;

V45L+L149R+H219I+V258P+A276S+H358T+A364G+S388K;

V45L+T70R+A117S+L149R+S214Q+V258P+A276S+L281V+H358D+A364G+S388K+G394A+C396V+D470T+R483V;

V45N+L149R+S214Q+V258P+A276S+A364G+S388K;

V45L+A117S+L149R+S214Q+H219V+V258P+A276S+H358D+A364G+S388K;

V45N+A117S+L149R+S214Q+V258P+A276S+H358D+A364G+S388K;

V45L+S92D+L149R+S214Q+V258P+A276S+A364G+S388K;

V45L+L149R+V258P+A276S+H358T+A364G;

V45L+A117S+L149R+S214Q+V258P+A276S+A364G+S388K+L478P;

V45L+A117S+L149R+S214Q+V258P+A276S+A364G+S388K+S481A;

V45L+A117S+L149R+S214Q+V258P+A276S+Q314A+A364G+S388K;

A25W+V45L+L149R+T211H+S214E+V258P+A276S+A364G;

A25W+V45L+L149R+S214E+H219V+V258P+A276S+A364G;

V45L+A117S+L149R+S214Q+V258P+A276S+H358D+A364G+S388K+S389V;

V45L+A117S+L149R+S214Q+V258P+A276S+H358D+A364G+S388K+V393H;

V45L+A117S+L149R+S214Q+V258P+A276Q+H358D+A364G+S388K;

V45L+A117S+N127D+L149R+S214Q+V258P+A276S+H358D+A364G+S388K;

V45L+L149R+W250Y+V258P+A276S+H358T+A364G+S388K;

D9E+V45L+L149R+V258P+A276S+H358T+A364G+S388K;

V45L+L149R+H219V+V258P+A276S+H358T+A364G+S388K;

V45L+A117S+L149R+S214Q+V258P+A276S+H358D+A364G+S388K+A472L;

A25W+V45L+L149R+V258P+A276S+A364G;

V45L+A117S+L149R+G187K+S214Q+V258P+A276S+A364G+S388K;

V45L+A117S+L149R+S214Q+V258P+A276S+A364G+S388K+L478N;

V45L+A117S+L149R+S214Q+V258P+A276S+A364G+S388K+V393R;

V45L+A117S+L149R+S214E+V258P+A276S+A364G+S388K;

V45L+A117S+L149R+S214Q+V258P+A276S+A364G+T366V+S388K;

V45L+A117S+L149R+S214Q+V258P+A276S+H358T+A364G+S388K;

D11E+A25W+V45L+L149R+S214E+V258P+A276S+A364G;

V45L+T70R+A117S+L149R+C161V+S214Q+V258P+A276S+L281V+H358D+A364G+S388K+G394A+D470T+R483V;

V45L+A117S+L149R+T211H+S214Q+V258P+A276S+H358D+A364G+S388K;

V45L+L149R+V258P+A276S+Q314A+H358T+A364G+S388K;

D11E+V45L+A117S+L149R+S214Q+V258P+A276S+A364G+S388K;

V45L+A117S+L149R+S214Q+V258P+A276S+A364G+S388K+G394Q;

V45L+A117S+L149R+S214Q+V258P+A276S+A364G+S388K+N395D;

V45L+A117S+L149R+S214Q+V258P+A276S+A364G+S388K+V393H;

V45L+A117S+L149R+S214Q+V258P+A276S+A364G+S388K+S501E;

V45L+A117S+L149R+S214Q+V258P+A276S+A364G+S388K+S528H;

V45L+T70R+A117S+L149R+S214Q+V258P+A276S+H358D+A364G+S388K+D470T+L530R;

V45L+A117S+L149R+S214Q+V258P+A276S+A364G+S388L;

V45L+T70R+A117S+L149R+S214Q+V258P+A276S+L281V+H358D+A364G+S388K+G394A+C396L+D470T+R483V;

V45L+L149R+T180Q+S214Q+V258P+A276S+A364G+S388K;

V45L+D113T+L149R+S214Q+V258P+A276S+A364G+S388K;

V45L+A117S+L149R+S214Q+V258P+A276S+H358D+A364G+S388K+T480L;

V45L+A93S+L149R+A252S+H334D;

V45L+A93S+L149R+V234P+A252S;

V45L+A117S+L149R+S214Q+V258P+A276S+H358D+A364G+S388H;

A25W+V45L+Q61R+L149R+S214E+V258P+A276S+A364G;

V45L+A117S+L149R+S214Q+T215K+V258P+A276S+H358D+A364G+S388K+V45L+A117S+L149R+S214Q+V258P+A276S+A364G+S388K+S389H;

V45L+A117S+L149R+S214Q+V258P+A276S+A364G+T366I+S388K;

V45L+A117S+L149R+S214Q+V258P+A276S+A364G+S388K+N474G;

A25W+V45L+L149R+S214E+V258P+A276S+L281V+A364G;

D9E+A25W+V45L+L149R+S214E+V258P+A276S+A364G;

V45L+A117S+L149R+S214Q+T215K+V258P+A276S+H358D+A364G+S388K+D470T+L530R;

V45L+A117S+L149R+S214Y+V258P+A276S+H358D+A364G+S388K;

V45L+A117S+L149R+S214Q+V258P+A276S+H358D+A364G+S388K+V393R;

V45L+Q61R+L149R+V258P+A276S+H358T+A364G+S388K;

V45L+L149R+S214Q+V258P+A276S+A364G+S388K;

V45L+D113T+L149R+V258P+A276S+H358T+A364G+S388K;

V45L+A117S+L149R+S214Q+V258P+A276S+A286S+A364G+S388K;

V45L+A117S+L149R+S214Q+V258P+A276S+A364G+S388R;

V45L+A117S+L149R+S214Q+V258P+A276S+A364G+S388K+A499Y;

V45L+A117S+L149R+S214Q+V258P+A276S+A364G+S388K+V500V;

V45L+Q61R+L149R+S214Q+V258P+A276S+A364G+S388K;

V45L+L149R+S214E+V258P+A276S+H358T+A364G;

V45L+A117S+L149R+S214Q+V258P+A276Q+A364G+S388K;

V45L+L149R+V258P+A276S+L281V+A364G;

V45L+A117S+L149R+S214Q+W250Y+V258P+A276S+A364G+S388K;

V45L+A117S+L149R+S214Q+V258P+A276S+A364G+S388K+T480N;

V45L+A117S+L149R+G187S+S214Q+V258P+A276S+A364G+S388K;

A25W+V45L+A117S+L149R+S214Q+V258P+A276S+A364G+S388K;

V45L+A117S+L149R+S214Q+V258P+A276S+A364G+S388K;

V45L+A117S+L149R+S214Q+V258P+A276S+A364G+S388K+N476N;

A25W+V45L+L149R+S214E+V258P+A276Q+A364G;

V45L+A117S+L149R+S214Q+V258P+A276S+A364G+S388K+N476D;

V45L+L149R+S214Q+V258P+A276S+L281V+A364G;

V45L+A93S+L149R+A252S+H334D+A340G;

V45L+A93S+L149R+A252S+H334D;

V45L+A93S+L149R+A252S+H334D;

V45L+L149R+A252S+H334D;

V45L+N127D+L149R+V258P+A276S+A364G+S388K;

V45L+L149R+V258P+A276S+H358T+A364G+S388K;

V45L+L149R+V258P+A276S+A364G+S388K;

D9E+V45L+A117S+L149R+S214Q+V258P+A276S+A364G+S388K;

V45L+A117S+L149R+S214Q+V258P+A276S+A364G+S388K+S481S;

V45L+L149R+N168G+V258P+A276S+A364G;

V45L+L149R+T180Q+V258P+A276S+H358T+A364G+S388K;

V45L+L149R+G187L+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

A25W+V45L+L149R+V258P+A276S+A364G+S388K;

V45L+L149R+S214Q+V258P+A276S+H358T+A364G;

V45L+L149R+S173Y+V258P+A276S+A364G;

V45L+A117S+L149R+S214Q+H219V+V258P+A276S+A364G+S388K;

V45L+A93S+L149R+A252S+H334D;

V45L+A93S+L149R+A252S+H334D+A340G;

V45L+L149R+S190E+D446T+R459V;

A25W+V45L+L149R+V258P+A276S+H358T;

A25W+V45L+S92D+L149R+S214E+V258P+A276S+A364G;

A25W+V45L+L149R+S214E+V258P+A276S+V350L+A364G;

A25W+V45L+L149R+S214Q+V258P+A276S+A364G;

V45L+L149R+N168G+V258P+A276S+A364G+S388K;

V45L+A117S+L149R+V258P+A276S+A364G+S388K;

A25W+V45L+L149R+V258P+A276S+H358T+A364G;

V45L+L149R+V258P+N260H+A276S+A364G+S388K;

V45L+L149R+T215I+V258P+A276S+A364G+S388K;

A25W+V45L+L149R+V258P+A276Q+A364G;

V45L+T70R+A117S+L149R+C161L+S214Q+V258P+A276S+L281V+H358D+A364G+S388K+G394A+D470T+R483V;

A25W+V45L+L149R+S214E+V258P+A276S;

V45L+A117S+L149R+S214Q+V258P+A276S+A286S+H358D+A364G+S388K;

V45L+L149R+T211H+S214Q+V258P+A276S+A364G+S388K;

V45L+Q61K+L149R+V258P+A276S+H358T+A364G;

V45L+L149R+V258P+A276S+Q314A+H358T+A364G;

V45L+L149R+T180Q+V258P+A276S+A364G+S388K;

V45L+L149R+V258P+A276S+Q314A+A364G+S388K;

A25W+V45L+L149R+V258P+A276S+A286S+A364G;

V45L+L149R+S190E+D446T;

V45L+L149R+V258P+A276S+A364G;

V45L+L149R+A170S+V258P+A276S+A364G;

V45L+L149R+V258P+A276Q+A364G;

V45L+T70R+A117S+L149R+S214Q+V258P+A276S+L281V+H358D+A364G+S388K+G394A+D470T+R483V+C522A;

V45L+L149R+H219I+V258P+A276S+H358T+A364G;

V45L+L149R+V258P+A276S+V350L+A364G+S388K;

D11E+V45L+L149R+V258P+A276S+A364G+S388K;

V45L+D113T+L149R+V258P+A276S+A364G+S388K;

V45L+L149R+G187K+V258P+A276S+A364G+S388K;

M28P+V45L+L149R+V258P+A276S+A364G+S388K;

V45L+Q61R+A117S+L149R+S214Q+V258P+A276S+H358D+A364G+S388K+D470T+R483V;

A25W+V45L+L149R+S214Q+V258P+A276S;

V45L+A117S+L149R+S214Q+T215I+V258P+A276S+A364G+S388K;

A25W+V45L+L149R+V258P+A276S+S388K;

A25W+V45L+L149R+S214E+H219I+V258P+A276S+A364G;

V45L+A93S+L149R+V234P+A252S+H334D;

V45L+L149R+T211H+V258P+A276S+H358T+A364G+S388K;

A25W+V45L+A117S+L149R+S214E+V258P+A276S+A364G;

V45L+L149R+S173Y+V258P+A276S+A364G+S388K;

A25W+V45L+L149R+V258P+A276S+Q314A+A364G;

V45L+L149R+G187L+V258P+A276S+H358T+A364G;

V45L+L149R+T215V+V258P+A276S+H358T+A364G;

V45L+T70R+L149R+V258P+A276S+A364G+S388K;

V45L+N127D+L149R+V258P+A276S+A364G;

V45L+A117S+L149R+S214Q+V258P+A276S+A364G+S388K+Q496R;

V45L+L149R+W250Y+V258P+A276S+A364G;

V45L+L149R+V258P+A276S+V350L+H358T+A364G;

D9E+V45L+L149R+V258P+A276S+H358T+A364G;

V45L+L149R+T211H+V258P+A276S+H358T+A364G;

A25W+V45L+L149R+W250Y+A276Q+A364G;

V45L+L149R+V258P+A276S+L281V+A364G+S388K;

V45L+L149R+V258P+A276S+S282N+H358T+A364G;

V45L+L149R+T215I+V258P+A276S+H358T+A364G;

A25W+V45L+L149R+S173Y+V258P+A276S+A364G;

V45L+Q61K+L149R+V258P+A276S+A364G+S388K;

V45L+L149R+T180Q+V258P+A276S+A364G;

V45L+A117S+L149R+A170S+S214Q+V258P+A276S+A364G+S388K;

V45L+Q61R+A117S+L149R+S214Q+V258P+A276S+A364G+S388K;

A25W+V45L+L149R+S214E+T215V+V258P+A276S+A364G;

V45L+A117S+L149R+S214Q+V258P+A276S+A364G+S388K+V497P;

V45L+L149R+V258P+A276S+Q314A+A364G;

V45L+A117S+L149R+V258P+A276S+A364G;

V45L+A117S+L149R+V258P+A276S+H358T+A364G+S388K;

V45L+L149R+N168G+V258P+A276S+H358T+A364G+S388K;

A25W+V45L+Q61K+L149R+V258P+A276S+A364G;

V45L+L149R+S214E+V258P+A276S+L281V+A364G;

V45N+L149R+V258P+A276S+A364G+S388K;

V45L+L149R+V258P+A276S+A286E+H358T+A364G;

D9E+V45L+L149R+V258P+A276S+A364G+S388K V45L+S92D+L149R+V258P+A276S+A364G+S388K;

V45L+A117S+L149R+T180Q+S214Q+V258P+A276S+A364G+S388K;

V45L+A117S+L149R+S214Q+V258P+A276S+A364G+S388H;

V45L+A117S+L149R+S214Q+T215V+V258P+A276S+A364G+S388K;

V45L+A117S+L149R+S214Q+V258P+A276S+V350L+A364G+S388K;

A25W+V45L+L149R+S214E+T215I+V258P+A276S+A364G;

V45L+N127D+L149R+V258P+A276S+H358T+A364G+S388K;

A25W+V45L+L149R+V258P+A276S+A286E+A364G;

V45L+L149R+N168G+V258P+A276S+L281V+A364G;

V45L+Q61R+L149R+V258P+A276S+H358T+A364G;

V45L+L149R+G187S+V258P+A276S+A364G+S388K;

V45L+L149R+V258P+A276S+L281V+Q314A+A364G;

V45L+L149R+V258P+A276S+L281V+H358T+A364G;

V45L+L149R+A170S+V258P+A276S+H358T+A364G;

V45L+L149R+A170S+V258P+A276S+A364G+S388K;

V45L+L149R+T215V+V258P+A276S+A364G+S388K;

V45L+L149R+V258P+A276Q+L281V+A364G;

V45L+T70R+A117S+L149R+S214Q+C243S+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V+C522V;

A25W+V45L+L149R+V258P+A276S+Q314A;

V45L+L149R+V258P+A276Q+H358T+A364G+S388K;

A25W+V45N+L149R+S214E+V258P+A276S+A364G;

V45L+L149R+V258P+A276S+A286E+A364G;

V45L+L149R+V258P+A276S+A286S+H358T+A364G;

A25W+V45L+L149R+G187S+V258P+A276S+A364G;

V45L+L149R+T180Q+V258P+A276S+L281V+A364G;

A25W+V45L+L149R+A276Q+L281V+A364G;

V45L+S92D+L149R+V258P+A276S+L281V+A364G;

V45L+Q61R+L149R+V258P+A276S+L281V+A364G;

V45L+L149R+A170S+V258P+A276S+L281V+A364G;

D11E+A25W+V45L+L149R+V258P+A276S+A364G;

A25W+V45L+Q61R+L149R+V258P+A276S+A364G;

A25W+V45L+L149R+T215V+V258P+A276S+A364G;

D9E+V45L+L149R+V258P+A276S+L281V+A364G;

A25W+V45L+L149R+V258P+A276S+V350L+A364G;

V45L+Q61K+A117S+L149R+S214Q+V258P+A276S+A364G+S388K;

V45L+A117S+L149R+S214Q+V258P+A276S+A364G+S388K+Y391V;

V45L+A117S+L149R+N168G+S214Q+V258P+A276S+A364G+S388K;

V45L+D113T+A117S+L149R+S214Q+V258P+A276S+A364G+S388K;

A25W+V45L+L149R+V258P+A276S+A286E;

A25W+V45L+L149R+V258P+A276S+L281V;

A25W+V45L+D113T+L149R+V258P+A276S;

V45L+L149R+H219V+V258P+A276S+L281V+A364G;

V45L+L149R+T180Q+V258P+A276S+H358T+A364G;

V45L+L149R+T215V+V258P+A276S+L281V+A364G;

A25W+V45L+L149R+V258P+A276S+S282N+A364G;

A25W+V45L+L149R+T215I+V258P+A276S+A364G;

A25W+V45L+L149R+H219I+V258P+A276S+A364G;

V45L+A117S+L149R+V258P+A276S+L281V+A364G;

D9E+A25W+V45L+L149R+V258P+A276S+A364G;

V45L+L149R+S214Y+V258P+A276S+A364G+S388K;

A25W+V45L+L149R+T180Q+V258P+A276S+A364G;

A25W+V45L+L149R+V258P+A276S+L281V+A364G;

V45L+N127D+L149R+V258P+A276S+L281V+A364G;

V45L+L149R+S173Y+V258P+A276S+H358T+A364G;

V45L+Q61R+L149R+V258P+A276S+A364G+S388K;

M28P+V45L+A117S+L149R+S214Q+V258P+A276S+A364G+S388K;

V45L+A117S+L149R+S214Q+V258P+A276S+A364G+S388A;

V45N+A117S+L149R+S214Q+V258P+A276S+A364G+S388K;

V45L+A117S+L149R+S214Q+V258P+A276S+A364G+S388K+T477K;

A25W+V45L+L149R+T180Q+V258P+A276S;

A25W+V45L+L149R+V258P+A276S+A286S;

A25W+V45L+L149R+G187S+S214E+V258P+A276S+A364G;

V45L+L149R+V258P+A276S+L281V+A286E+A364G;

A25W+V45L+L149R+W250Y+V258P+A276S+A364G;

A25W+V45L+L149R+V258P+N260H+A276S+A364G;

V45L+L149R+V258P+A276S+L281V+V350L+A364G;

A25W+V45L+L149R+A170S+V258P+A276S+A364G;

V45L+L149R+V258P+A276S+L281V+A286S+A364G;

V45L+L149R+V258P+A276S+L281V+S282N+A364G;

D11E+V45L+L149R+V258P+A276S+L281V+A364G;

V45L+Q61K+L149R+V258P+A276S+L281V+A364G;

V45L+L149R+T215I+V258P+A276S+L281V+A364G;

A25W+V45L+S92D+L149R+V258P+A276S+A364G;

V45L+L149R+S214Y+V258P+A276S+L281V+A364G;

V45L+L149R+T211H+V258P+A276S+L281V+A364G;

A25W+V45L+L149R+G187L+V258P+A276S+A364G;

V45L+S92D+L149R+V258P+A276S+H358T+A364G;

V45L+L149R+G187L+A276Q+L281V+A364G;

V45L+L149R+A276Q+L281V+V350L+A364G;

V45L+L149R+A276Q+L281V+A364G+S388K;

V45L+A117S+L149R+S214Y+V258P+A276S+A364G+S388K;

A25W+V45L+S92D+L149R+V258P+A276S;

A25W+V45L+L149R+G187L+V258P+A276S;

A25W+V45L+L149R+W250Y+V258P+A276S;

V45L+D113T+L149R+V258P+A276S+A364G;

V45L+L149R+G187K+V258P+A276S+H358T+A364G;

V45L+L149R+N163H+V258P+A276S+L281V+A364G;

A25W+V45L+T70R+L149R+V258P+A276S+A364G;

V45L+L149R+S173Y+V258P+A276S+L281V+A364G;

V45L+L149R+G187L+V258P+A276S+L281V+A364G;

V45L+L149R+G187K+V258P+A276S+L281V+A364G;

V45L+L149R+W250Y+V258P+A276S+H358T+A364G;

V45L+L149R+T211H+V258P+A276S+A364G+S388K;

V45L+L149R+N163H+V258P+A276S+A364G+S388K;

V45L+T70R+L149R+A276Q+L281V+A364G;

V45L+S92D+L149R+A276Q+L281V+A364G;

V45L+D113T+L149R+A276Q+L281V+A364G;

V45L+L149R+A276Q+L281V+Q314A+A364G;

V45L+A117S+L149R+S214Q+V258P+A276S+A364G+S388K+S389V;

A25W+V45L+Q61K+L149R+V258P+A276S;

D9E+A25W+V45L+L149R+V258P+A276S;

V45L+T70R+L149R+V258P+A276S+A364G;

V45L+L149R+V258P+A276S+A286S+A364G+S388K;

A25W+V45L+L149R+S214Y+V258P+A276S+A364G;

A25W+V45L+D113T+L149R+V258P+A276S+A364G;

V45L+T70R+L149R+V258P+A276S+L281V+A364G;

A25W+V45L+N127D+L149R+V258P+A276S+A364G;

V45L+T70R+L149R+V258P+A276S+H358T+A364G;

V45L+L149R+N163H+V258P+A276S+A364G;

V45L+L149R+T180Q+A276Q+L281V+A364G;

V45L+L149R+A276Q+L281V+A286S+A364G;

A25W+V45L+L149R+A170S+V258P+A276S;

A25W+V45L+T70R+L149R+V258P+A276S;

A25W+V45L+L149R+S214E+V258P+A276S+A286S+A364G;

V45L+L149R+V258P+A276S+A286S+H358T+A364G+S388K;

V45L+L149R+H219I+V258P+A276S+L281V+A364G;

A25W+V45L+A117S+L149R+V258P+A276S+A364G;

A25W+V45L+L149R+N168G+V258P+A276S+A364G;

V45L+L149R+N168G+A276Q+L281V+A364G;

V45L+L149R+S173Y+A276Q+L281V+A364G;

V45L+L149R+W250Y+A276Q+L281V+A364G;

V45L+A117S+L149R+S173Y+S214Q+V258P+A276S+A364G+S388K;

A25W+V45L+L149R+A276Q+A286E+A364G;

A25W+V45L+L149R+A276Q+A364G+S388K;

V45L+N127D+L149R+V258P+A276S+H358T+A364G;

A25W+V45L+L149R+V258P+A276S;

V45L+N127D+L149R+A276Q+L281V+A364G;

V45L+L149R+A276Q+L281V+A286E+A364G;

V45L+A117S+L149R+S214Q+V258P+A276S+A364G+S388K+V500T;

A25W+V45L+A117S+L149R+V258P+A276S;

A25W+V45L+L149R+H219V+V258P+A276S+A364G;

V45L+L149R+S173Y+V258P+A276S+H358T+A364G+S388K;

D11E+V45L+L149R+V258P+A276S+H358T+A364G;

V45L+A79C+L149R+V258P+A276S+N355G+A364G+S388K;

A24K+V45L+L149R+V258P+A276S+A364G;

V45L+L149R+V258P+A276S+S282N+A364G;

V45L+A117S+L149R+A276Q+L281V+A364G;

V45L+L149R+N260H+A276Q+L281V+A364G;

V45L+A117S+L149R+T211H+S214Q+V258P+A276S+A364G+S388K;

A25W+V45L+L149R+N168G+V258P+A276S;

A25W+V45L+L149R+G187S+V258P+A276S;

A25W+V45L+L149R+V258P+A276S+V350L;

A25W+V45L+Q61R+L149R+V258P+A276S;

A25W+V45L+L149R+G187L+S214E+V258P+A276S+A364G;

V45L+L149R+G187S+V258P+A276S+L281V+A364G;

A25W+V45L+L149R+N163H+V258P+A276S+A364G;

A25W+V45L+L149R+G187K+V258P+A276S+A364G;

V45N+L149R+V258P+A276S+L281V+A364G;

V45L+L149R+A276Q+L281V+A364G;

V45L+L149R+V258P+N260H+A276S+H358T+A364G;

V45L+L149R+A276Q+S282N+A364G;

V45L+S92D+A117S+L149R+S214Q+V258P+A276S+A364G+S388K;

A25W+V45L+L149R+V258P+A276S+S282N;

A25W+V45L+L149R+S173Y+V258P+A276S;

A25W+V45L+L149R+A276Q+A286S+A364G;

V45L+D113T+L149R+V258P+A276S+L281V+A364G;

V45L+L149R+S214Q+V258P+A276S+L281V+A364G+S388K;

V45L+D113T+L149R+V258P+A276S+H358T+A364G;

A25W+V45L+L149R+T215V+V258P+A276S;

V45L+L149R+N168G+V258P+A276S+H358T+A364G;

V45L+A117S+L149R+S214Q+V258P+A276S+A364G+S388K+A472L;

V45L+A117S+L149R+S214Q+V258P+A276S+A364G+S388K+S473K;

A25W+V45L+L149R+T215I+V258P+A276S;

A25W+V45L+L149R+T211H+V258P+A276S;

A25W+V45L+L149R+V258P+A276Q;

V45L+L149R+S214Q+T215V+V258P+A276S+A364G+S388K;

V45L+L149R+N163H+V258P+A276S+H358T+A364G+S388K;

V45L+A117S+L149R+S214Q+V258P+A276S+A364G+S388K+A499L;

V45L+L149R+V258P+A276S+V350L+A364G;

V45L+A79C+A117S+L149R+V258P+A276S+A364G+S388K;

A25W+V45L+D113T+L149R+A276Q+A364G;

A25W+V45N+L149R+V258P+A276S+A364G;

V45L+T70R+L149R+A276Q+A364G;

A25W+V45L+L149R+H219I+V258P+A276S;

V45L+A117S+L149R+S214Q+V258P+A276S+A364G+S388K+V498I;

M28P+V45L+L149R+V258P+A276S+A364G;

A25W+V45L+T70R+L149R+A276Q+A364G;

A25W+V45L+L149R+A276Q+Q314A+A364G;

V45L+L149R+A170S+V258P+A276S+H358T+A364G+S388K;

A25W+V45L+L149R+S214Y+V258P+A276S;

V45L+L149R+S214Y+V258P+A276S+H358T+A364G;

V45L+L149R+A276Q+L281V+H358T+A364G;

A25W+V45L+L149R+H219V+V258P+A276S;

V45L+A117S+L149R+N163H+S214Q+V258P+A276S+A364G+S388K;

D11E+A25W+V45L+L149R+V258P+A276S;

V45L+A117S+L149R+S214Q+V258P+A276S+A364G+S388K+N527H;

A25W+V45L+A117S+L149R+A276Q+A364G;

V45L+L149R+V258P+L281V+A364G;

A25W+V45L+L149R+G187L+A276Q+A364G;

V45N+L149R+V258P+A276S+H358T+A364G;

V45L+A117S+L149R+S214Q+V258P+A276S+N355G+H358D+A364G+S388K+D470T+R483V;

V45L+A117S+L149R+S214Q+V258P+A276S+H358D+A364G+S388H+D470T+R483V A25W+V45L+L149R+A276Q+A364G;

V45L+A117S+L149R+S214Q+V258P+A276S+H358D+A364G+S388K+D470T+R483V+V497E;

A25W+V45L+L149R+N163H+A276Q+A364G;

A25W+V45L+L149R+S173Y+A276Q+A364G;

A25W+V45L+L149R+A276Q+S282N+A364G;

A25W+V45L+L149R+A276Q+H358T+A364G;

A25W+V45L+L149R+T180Q+A276Q+A364G;

V45L+A117S+L149R+V258P+A276S+H358T+A364G;

V45L+L149R+A276Q+Q314A+A364G;

V45L+L149R+G187K+V258P+A364G;

V45L+L149R+A276Q+A286E+A364G;

A25W+V45L+N127D+L149R+A276Q+A364G;

A25W+V45L+L149R+A170S+A276Q+A364G;

A25W+V45L+N127D+L149R+V258P+A276S;

V45L+A117S+L149R+S214Q+V258P+A276S+A364G+S388K+T477V;

V45L+A117S+L149R+S214Q+V258P+A276S+A364G+S388K+G503L;

A25W+M28P+V45L+L149R+V258P+A276S+A364G;

V45L+L149R+A276Q+H358T+A364G;

A25W+V45L+L149R+N168G+A276Q+A364G;

A25W+V45L+L149R+N260H+A276Q+A364G;

V45L+A117S+L149R+G187L+S214Q+V258P+A276S+A364G+S388K;

V45L+A117S+L149R+N163H+S214Q+V258P+A276S+H358D+A364G+S388K+D470T+L530R;

V45L+A117S+L149R+S214Q+T215K+V258P+A276S+A364G+S388K;

A25W+V45L+L149R+G187K+V258P+A276S;

A25W+M28P+V45L+L149R+V258P+A276S;

A25W+V45N+L149R+V258P+A276S;

V45L+A117S+L149R+S214Q+V258P+A276S+N355G+H358D+A364G+S388K+D470T;

V45L+N127D+L149R+A276Q+A364G;

V45L+L149R+W250Y+A276Q+A364G;

V45L+L149R+W250Y+V258P+A364G;

V45L+L149R+N163H+A276Q+L281V+A364G;

V45L+A117S+L149R+S214Q+V258P+A276S+A364G+Q385C+S388K;

A25W+V45L+L149R+S214E+T215K+V258P+A276S+A364G;

M28P+V45L+L149R+V258P+A276S+L281V+A364G;

V45L+L149R+A276Q+A364G+S388K;

A25W+V45L+L149R+V258P+A364G;

M28P+V45L+L149R+V258P+A276S+H358T+A364G;

V45L+A117S+L149R+S214Q+V258P+A276S+N355G+H358D+A364G+S388K;

V45L+T70R+A117S+L149R+S214Q+V258P+A276S+L281V+H358D+A364G+S388K+G394A+D470T+R483V+C540V;

V45L+L149R+N168G+A276Q+A364G;

V45L+A117S+L149R+A276Q+A364G;

V45L+L149R+N163H+V258P+A276S+H358T+A364G;

V45L+L149R+T215K+V258P+A276S+H358T+A364G;

A24K+V45L+L149R+A276Q+A364G;

V45L+L149R+A276Q+A286S+A364G;

V45L+L149R+V258P+A364G;

V45L+L149R+H219I+V258P+A276S+A364G+S388K;

V45L+L149R+A170S+A276Q+A364G;

V45L+L149R+T180Q+A276Q+A364G;

V45L+L149R+A276Q+V350L+A364G;

V45L+L149R+V258P+S282N+A364G;

V45L+L149R+A276Q+A364G;

V45L+L149R+A276S+A364G;

V45L+L149R+T215K+V258P+A276S+A364G+S388K;

V45L+A117S+L149R+S214Q+V258P+A276S+N355G+A364G+S388K+D470T V45L+L149R+V258P+A276S;

A25W+V45L+L149R+A276S+A364G;

V45L+L149R+G187S+V258P+A364G;

V45L+S92D+L149R+V258P+A276S+H358T+A364G+S388K;

A25W+L149R+V258P+A276S+A364G;

V45L+L149R+G187L+A276Q+A364G;

A25W+V45L+V258P+A276S+A364G;

V45L+L149R+N260H+A276Q+A364G;

V45L+L149R+W250Y+V258R+A364G;

V45L+L149R+N163H+A276Q+A364G;

V45L+S92D+L149R+A276Q+A364G;

V45L+L149R+S173Y+A276Q+A364G;

V45L+S92D+L149R+V258P+A364G;

V45L+L149R+W250Y+V258P+A276S+L281V+A364G;

V45L+L149R+L281V+A364G;

V45L+D113T+L149R+A276Q+A364G;

L149R+V258P+A276S+A364G;

V45L+L149R+G187S+V258R+A364G;

V45L+V258P+A276S+A364G;

V45L+L149R+V258R+A364G+S388K;

A25W+V45L+L149R+A364G;

V45L+L149R+G187K+V258R+A364G;

V45L+L149R+V258P;

V45L+L149R+V258R+S282N+A364G;

V45L+L149R;

V45L+L149R+V258R+L281V+A364G;

V45L+L149R+A364G;

V45L+L149R+S214Q+V258P+A276S+N355G+A364G+S388K;

V45L+S92N+L149R+V258P+A364G;

V45L+A117S+L149R+S214Q+V258P+A276S+A286E+H358D+A364G+S388K;

A25W+V45L+L149R+S214E+V258P+A276S+N355G+A364G;

V45L+L149R+G187L+V258P+A276S+A364G;

V45L+L149R+V258R+A364G;

V45L+L149R+V258P+A364G+S388K;

A25W+V45L+L149R+A276Q+V350L+A364G;

V45L+L149R+H219V+V258P+A276S+A364G+S388K;

V45L+L149R+G187K+A364G;

V45L+L149R+G187S+A364G;

V45L+A117S+L149R+S214Q+V258P+A276S+N355G+A364G+S388K;

V45L+A117S+L149R+S214Q+V258P+A276S+H358D+A364G+Y386G+S388K+D470T+R483V;

V45L+L149R+W250Y;

V45L+S92N+L149R+V258R+A364G;

A25W+V45L+L149R+V258P+A276S+N355G+A364G;

V45L+L149R+V258P+A276S+N355G+A364G+S388K;

V45L+L149R+G187S;

V45N+L149R;

V45L+L149R+S282N;

L149R+A364G;

Y386G;

V258P+A276Q;

V258P+A276S;

V45L+A117S+L149R+S214Q+V258P+A276S+H358D+A364G+S388K+G394D+D470T+R483V;

A25W+L149R;

V45L+A276S;

V45L+S92D+L149R;

A25W+A276S;

Q385C;

V45L+L149R+W250Y+A364G;

V45L+A276Q;

V45L+V258P;

V45L+A117S+L149R+S214Q+V258P+A276S+H358D+A364G+Y386G+S388K;

A25W+V258P;

A25W+A276Q;

L149R+W250Y;

V45L+L149R+V258P+A276S+L281V+N355G+A364G;

V45L+S388K;

V45L+L149R+V258P+A276S+N355G+H358T+A364G;

V45L+A364G;

V45N+V258P;

A276S;

V258P+S388K;

A25W+V45L;

A276Q+S388K;

A276C;

A276Q;

A276F;

W250Y+A276Q;

A79C;

A276S+S388K;

A276Q+L281V;

V45L+L149R+V258P+A276S+N355G+A364G;

A276N;

W250Y+A276S;

A25W+S388K;

A276S+L281V;

G187S+A276Q;

A25W+V45L+L149R+V258P+A276S+N355G;

V45L;

S92D+A276S;

S19T;

L149R+V258R;

V45N+S388K;

A25W+V45L+S92D+L149R+A276Q+A364G;

V45L+A117S+L149R+S214Q+V258P+A276S+A364G+S388K+D470T+S501E H358D;

A25W+A364G;

V258P+L281V;

L149R;

V45L+L149R+H219V+V258P+A276S+H358T+A364G;

A25W;

V258R+A276S;

V45N;

G187S+A276S;

G187K+A276S;

I469T;

V45L+S92D;

V45L+L281V;

V45N+W250Y;

V258R+A276Q;

V45L+V258R;

W250Y+S388K;

A25W+V45L+L149R+A276Q+N355G+A364G;

V45N+G187K;

A25W+V45L+L149R+S214E+V258P+A276S+Q314A+A364G;

S388A;

A276T;

A25W+V258R;

G187K+S388K;

A25W+S92D;

L281V+S388K;

V45N+V258R;

S388H;

S92D;

D525L;

S214Q;

L281V+A364G;

A364G;

S388L;

S388R;

S473G;

S92D+A364G;

A25W+G187S;

A25W+G187K;

W250Y+V258P;

S92D+V258P S389V;

A20H;

W495E;

G187K+W250Y;

Y386Y;

V393H;

G187L;

Q61K;

V45N+S92D;

G187S+A364G;

G187S+L281V;

G187S;

A286E;

A276Y;

V258P;

Q496E;

G394A;

V393R;

G187S+S388K;

T366V; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by TSA of at least 1° C. compared to the xylanase of SEQ ID NO: 1.

In one aspect, the present invention relates to xylanase variants comprises at least one of the following substitutions or combinations of substitutions:

V45L+Q61K+T70R+A117S+L149R+S214Q+V258P+A276S+L281V+A286E+H358D+A364G+Q384A+S388K+V393H+D470T+R483V;

V45L+Q61K+T70R+A117S+L149R+S214Q+V258P+A276S+L281V+H358D+A364G+Q384A+S388K+G394Q+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+A286E+H358D+A364G+Q384A+S388K+V393H+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+A79C+A117S+L149R+S214Q+V258P+A276S+H358D+A364G+S388K+D470T+L530R;

V45L+A79C+A117S+L149R+S214Q+V258P+A276S+H358D+A364G+S388K+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+Q61K+A117S+L149R+S214Q+V258P+A276S+L281V+H358D+A364G+S388K+G394A+D470T+R483V+L530R;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+Q61K+A117S+L149R+S214Q+V258P+A276S+L281V+H358D+A364G+S388K+G394A+D470T+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+D470T+R483V+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+D470T+R483V+L530R;

V45L+A117S+L149R+G187K+S214Q+V258P+A276S+A286E+H358D+A364G+Q384A+S388K+V393H+D470T+R483V;

V45L+A117S+L149R+G187K+S214Q+V258P+A276S+A286E+H358D+A364G+Q384A+S388K+N395D+D470T+R483V;

V45L+A117S+L149R+G187K+S214E+T215I+V258P+A276S+A286E+H358D+A364G+S388K+V393H+D470T+R483V;

V45L+A117S+L149R+G187K+S214E+T215I+V258P+A276S+A286E+H358D+A364G+S388K+N395D+D470T+R483V;

V45L+A117S+L149R+S214Q+V258P+A276S+H358D+A364G+Q385C+S388K+D470T+R483V;

V45L+Q61K+A117S+L149R+S214Q+V258P+A276S+H358D+A364G+S388K+D470T+R483V;

V45L+A79C+A117S+L149R+S214Q+V258P+A276S+A364G+S388K+D470T+R483V;

V45L+A117S+L149R+S214Q+V258P+A276S+L281V+H358D+A364G+S388K+D470T+R483V;

V45L+A117S+L149R+S214Q+V258P+A276S+H358D+A364G+T366I+S388K+D470T+R483V;

V45L+T70R+A117S+L149R+S214Q+V258P+A276S+L281V+H358D+A364G+S388K+G394A+D470T+R483V;

V45L+A117S+L149R+S214Q+V258P+A276S+H358D+A364G+Q384A+S388K+D470T+L530R;

V45L+Q61K+A117S+L149R+S214Q+V258P+A276S+H358D+A364G+S388K+D470T+L530R;

V45L+T70R+A117S+L149R+S214Q+V258P+A276S+H358D+A364G+S388K+D470T+R483V;

V45L+A117S+L149R+S214Q+V258P+A276S+H358D+A364G+S388K+V393H+D470T+R483V;

V45L+A117S+L149R+S214Q+V258P+A276S+H358D+A364G+S388K+G394A+D470T+R483V;

V45L+A117S+L149R+S214Q+V258P+A276S+H358D+A364G+S388K+V393H+D470T+L530R;

V45L+A117S+L149R+S214Q+V258P+A276S+A286E+H358D+A364G+S388K+D470T+R483V;

D11E+V45L+A117S+L149R+S214Q+V258P+A276S+H358D+A364G+S388K+D470T+R483V;

V45L+T70R+A117S+L149R+S214Q+V258P+A276S+L281V+H358D+A364G+S388K+G394A+C468V+D470T+R483V;

V45L+A79C+A117S+L149R+S214Q+V258P+A276S+H358D+A364G+S388K+D470T;

V45L+A79C+L149R+S214E+V258P+A276S+A364G+S388K;

V45L+A117S+L149R+G187L+S214Q+V258P+A276S+H358D+A364G+S388K+D470T+L530R;

V45L+A117S+L149R+S214Q+V258P+A276S+H358D+A364G+Q384A+S388K+D470T+R483V;

V45L+A117S+L149R+G187K+S214Q+V258P+A276S+H358D+A364G+S388K+D470T+R483V;

V45L+A117S+L149R+S214Q+V258P+A276S+L281V+H358D+A364G+S388K+D470T+L530R;

V45L+A117S+L149R+S214E+V258P+A276S+H358D+A364G+S388K+D470T+R483V;

V45L+A117S+L149R+G187L+S214Q+V258P+A276S+H358D+A364G+S388K+D470T+R483V;

V45L+A117S+L149R+S214Q+V258P+A276S+H358D+A364G+S388K+G394Q+D470T+R483V;

V45L+A117S+L149R+S214Q+V258P+A276S+H358D+A364G+S388K+G394A+D470T+L530R;

V45L+A117S+L149R+S214Q+V258P+A276S+H358D+A364G+T366V+S388K+D470T+L530R;

V45L+A117S+L149R+S214Q+V258P+A276S+H358D+A364G+S388K+G394Q+D470T+L530R;

V45L+A117S+L149R+S214Q+V258P+A276S+S282N+H358D+A364G+S388K+D470T+R483V V45L+A117S+L149R+S214Q+V258P+A276S+H358D+A364G+S388K+N395D+D470T+R483V;

V45L+A117S+L149R+S214Q+V258P+A276S+H358D+A364G+S388K+D470T+N476D+R483V;

V45L+A117S+L149R+S214Q+V258P+A276S+V350L+H358D+A364G+S388K+D470T+R483V;

V45L+A117S+L149R+S214Q+V258P+A276S+H358D+A364G+S388K+D470T+T477K+R483V;

V45L+A117S+L149R+S214Q+V258P+A276S+H358D+A364G+S388K+D470T+R483V+Q496R;

V45L+T70R+A117S+L149R+S214Q+V258P+A276S+L281V+H358D+A364G+S388K+G394A+C468A+D470T+R483V;

V45L+A117S+L149R+S214Q+V258P+A276S+H358D+A364G+S388K+D470T+R483V+W495E;

V45L+A117S+L149R+S214Q+V258P+A276S+A286E+H358D+A364G+S388K+D470T+L530R;

V45L+A117S+L149R+G187K+S214Q+V258P+A276S+H358D+A364G+S388K+D470T+L530R;

V45L+A117S+L149R+S214Q+V258P+A276S+H358D+A364G+S388K+Y391V+D470T+R483V;

V45L+A117S+L149R+S214Q+V258P+A276S+H358D+A364G+S388K+D470T+R483V;

D9E+V45L+A117S+L149R+S214Q+V258P+A276S+H358D+A364G+S388K+D470T+L530R;

V45L+A117S+L149R+S214Q+V258P+A276S+H358D+A364G+S388K+D470T+T477V+R483V;

V45L+A117S+L149R+S214Q+V258P+A276S+H358D+A364G+S388K+D470T+N476N+R483V;

V45L+A117S+L149R+S214Q+V258P+A276S+H358D+A364G+S388K+V393R+D470T+R483V;

V45L+A117S+L149R+S214Q+V258P+A276S+H358D+A364G+S388K+D470T+T480L+L530R;

V45L+A117S+L149R+S214Q+V258P+A276S+H358D+A364G+S388K+D470T+S481A+L530R;

V45L+A117S+L149R+S214Q+W250Y+V258P+A276S+H358D+A364G+S388K+D470T+R483V;

V45L+A117S+L149R+S214Q+V258P+A276S+H358D+A364G+S388K+D470T+S481S+R483V;

V45L+A117S+L149R+S173Y+S214Q+V258P+A276S+H358D+A364G+S388K+D470T+R483V;

V45L+T70R+A117S+L149R+S214Q+C243V+V258P+A276S+L281V+H358D+A364G+S388K+G394A+D470T+R483V;

V45L+A117S+L149R+T180Q+S214Q+V258P+A276S+H358D+A364G+S388K+D470T+L530R;

D11E+V45L+A117S+L149R+S214Q+V258P+A276S+H358D+A364G+S388K+D470T+L530R;

V45L+A117S+L149R+S214E+V258P+A276S+H358D+A364G+S388K+D470T+L530R;

V45L+A117S+L149R+S214Q+V258P+A276S+H358D+A364G+S388K+D470T+N476D+L530R;

V45L+A117S+L149R+S214Q+V258P+A276S+A286S+H358D+A364G+S388K+D470T+R483V;

V45L+A117S+L149R+S214Q+V258P+A276S+H358D+A364G+S388K+D470T+R483V+Q496Q;

V45L+A117S+L149R+S214Q+V258P+A276Q+H358D+A364G+S388K+D470T+R483V;

V45L+A117S+L149R+S214Q+V258P+A276S+H358D+A364G+S388K+D470T+R483V+Q496E;

V45L+Q61R+A117S+L149R+S214Q+V258P+A276S+H358D+A364G+S388K+D470T+L530R;

V45L+A117S+L149R+S214Q+V258P+A276S+Q314A+H358D+A364G+S388K+D470T+L530R;

V45L+A117S+L149R+S214Q+V258P+A276Q+H358D+A364G+S388K+D470T+L530R;

V45L+A117S+L149R+S214Q+V258P+A276S+H358D+A364G+S388K+D470T+L478P+R483V;

A25W+V45L+A117S+L149R+S214Q+V258P+A276S+H358D+A364G+S388K+D470T+R483V;

V45L+A117S+L149R+T180Q+S214Q+V258P+A276S+H358D+A364G+S388K+D470T+R483V;

V45L+A117S+L149R+S214Q+V258P+A276S+H358D+A364G+S388K+D470T+R483V+L484K;

V45L+A117S+L149R+S214Q+V258P+A276S+H358D+A364G+S388K+D470T+N474G+L530R;

V45L+A117S+L149R+S214Q+V258P+A276S+H358D+A364G+S388K+N395D+D470T+L530R;

V45L+A117S+L149R+S214Q+V258P+A276S+H358D+A364G+T366V+S388K+D470T+R483V;

V45L+A117S+L149R+N168G+S214Q+V258P+A276S+H358D+A364G+S388K+D470T+R483V;

V45L+A117S+L149R+S214Q+V258P+A276S+H358D+A364G+S388K+D470T+R483V+L484S;

V45L+A117S+L149R+S214Q+V258P+A276S+H358D+A364G+S388K+D470T+L530R;

V45L+A117S+L149R+S214Q+V258P+A276S+H358D+A364G+S388K+D470T+R483V+L484D;

V45L+A117S+L149R+S214Q+V258P+A276S+H358D+A364G+S388K+D470T+R483V+S528H;

V45L+A117S+L149R+S214Q+V258P+A276S+H358D+A364G+S388K+D470T+R483V+S501N;

V45L+A117S+L149R+S173Y+S214Q+V258P+A276S+H358D+A364G+S388K+D470T+L530R;

V45L+A117S+L149R+S214Q+V258P+A276S+H358D+A364G+S388K+D470T+R483V+A499L;

V45L+S92D+A117S+L149R+S214Q+V258P+A276S+H358D+A364G+S388K+D470T+R483V;

V45L+A117S+L149R+S214Q+V258P+A276S+Q314A+H358D+A364G+S388K+D470T+R483V;

V45L+A79C+L149R+S214Q+V258P+A276S+A364G+S388K;

V45L+A117S+L149R+S214Q+V258P+A276S+N355G+H358D+A364G+S388K+D470T+L530R;

V45L+A117S+L149R+S214Q+V258P+A276S+V350L+H358D+A364G+S388K+D470T+L530R;

V45L+A117S+L149R+S214Q+V258P+A276S+H358D+A364G+S388K+D470T+S481S+L530R;

V45L+A117S+L149R+S214Q+V258P+A276S+H358D+A364G+S388K+D470T+Q496E+L530R;

V45L+A117S+L149R+N168G+S214Q+V258P+A276S+H358D+A364G+S388K+D470T+L530R;

V45L+A117S+L149R+S214Q+V258P+A276S+H358D+A364G+S388K+D470T+V498I+L530R;

V45L+A117S+L149R+S214Q+V258P+A276S+H358D+A364G+S388K+D470A+R483V;

V45N+A117S+L149R+S214Q+V258P+A276S+H358D+A364G+S388K+D470T+R483V;

V45L+A117S+L149R+S214Q+V258P+A276S+H358D+A364G+S388K+D470T+R483V+A499Y;

V45L+A79C+L149R+V258P+A276S+Q314A+A364G+S388K;

V45L+A79C+L149R+V258P+A276S+L281V+A364G+S388K;

V45L+A117S+L149R+S214Q+V258P+A276S+A286S+H358D+A364G+S388K+D470T+L530R;

V45L+A117S+L149R+S214Q+V258P+A276S+H358D+A364G+S388K+D470T+S501N+L530R;

V45L+A117S+L149R+S214Q+V258P+A276S+H358D+A364G+S388K+D470T+N476N+L530R;

V45L+A117S+L149R+S214Q+V258P+A276S+H358D+A364G+S388K+D470T+T477V+L530R;

V45L+A117S+L149R+S214Q+V258P+A276S+H358D+A364G+S388H+D470T+L530R;

V45L+A117S+L149R+S214Q+V258P+A276S+H358D+A364G+S388K+D470T+L478N+L530R;

V45L+S92D+A117S+L149R+S214Q+V258P+A276S+H358D+A364G+S388K+D470T+L530R;

V45L+A117S+L149R+S214Q+V258P+A276S+H358D+A364G+S388K+D470T+V500V+L530R;

V45L+A117S+L149R+S214Q+V258P+A276S+H358D+A364G+S388K+D470T+R483V+V500V;

V45L+A117S+L149R+S214Q+V258P+A276S+H358D+A364G+S388K+D470T+L478P+L530R;

V45L+A117S+L149R+S214Q+V258P+A276S+H358D+A364G+S388K+D470T+R483V+S501E;

V45L+A117S+L149R+S214Q+V258P+A276S+H358D+A364G+S388K+D470T+D525L+L530R;

V45L+A117S+L149R+S214Q+V258P+A276S+H358D+A364G+S388K+D470T+G526T+L530R;

V45L+A117S+L149R+S214Q+V258P+A276S+H358D+A364G+S388K+D470T+N527H+L530R;

V45L+A117S+L149R+S214Q+V258P+A276S+H358D+A364G+S388K+D470T+T477K+L530R;

M28P+V45L+A117S+L149R+S214Q+V258P+A276S+H358D+A364G+S388K+D470T+L530R;

D9E+V45L+A117S+L149R+S214Q+V258P+A276S+H358D+A364G+S388K+D470T+R483V;

V45L+A117S+L149R+S214Q+V258P+A276S+H358D+A364G+S388K+D470T+S501E+L530R;

V45L+A117S+L149R+S214Q+W250Y+V258P+A276S+H358D+A364G+S388K+D470T+L530R;

V45L+A117S+L149R+S214Q+V258P+A276S+H358D+A364G+S388K+D470T+A499L+L530R;

V45L+A117S+L149R+S214Q+V258P+A276S+H358D+A364G+S388K+D470T+A472I+L530R;

V45L+A117S+N127D+L149R+S214Q+V258P+A276S+H358D+A364G+S388K+D470T+R483V;

V45L+A117S+L149R+S214Q+V258P+A276S+H358D+A364G+S388K+D470T+V500T+L530R;

V45L+A117S+L149R+S214Q+V258P+A276S+H358D+A364G+S388K+D470T+R483V+N527H;

V45N+A117S+L149R+S214Q+V258P+A276S+H358D+A364G+S388K+D470T+L530R;

V45L+A117S+L149R+S214Q+V258P+A276S+H358D+A364G+S388K+D470T+T480N+L530R;

V45L+A117S+L149R+S214Q+V258P+A276S+H358D+A364G+S388K+D470T+R483V+V497P;

V45L+A117S+L149R+S214Q+V258P+A276S+H358D+A364G+S388K+D470T+R483V+V498I;

V45L+A79C+L149R+S173Y+V258P+A276S+A364G+S388K;

V45L+A117S+L149R+S214Q+V258P+A276S+H358D+A364G+S388K+D470T+T480N+R483V;

V45L+A117S+L149R+S214Q+H219V+V258P+A276S+H358D+A364G+S388K+D470T+L530R;

V45L+A117S+L149R+S214Q+V258P+A276S+H358D+A364G+S388A+D470T+L530R;

M28P+V45L+A117S+L149R+S214Q+V258P+A276S+H358D+A364G+S388K+D470T+R483V;

V45L+A117S+L149R+A170S+S214Q+V258P+A276S+H358D+A364G+S388K+D470T+R483V;

V45L+A79C+L149R+G187K+V258P+A276S+A364G+S388K;

V45L+A117S+L149R+S214Q+V258P+A276S+H358D+A364G+S388K+S389H+D470T+L530R;

V45L+D113T+A117S+L149R+S214Q+V258P+A276S+H358D+A364G+S388K+D470T+L530R;

V45L+A117S+L149R+N163H+S214Q+V258P+A276S+H358D+A364G+S388K+D470T+R483V;

V45L+A117S+L149R+S214Q+V258P+A276S+H358D+A364G+S388A+D470T+R483V;

V45L+A117S+L149R+S214Q+V258P+A276S+H358D+A364G+S388L+D470T+R483V;

V45L+Q61R+A79C+L149R+V258P+A276S+A364G+S388K;

V45L+A79C+L149R+N168G+V258P+A276S+A364G+S388K;

V45L+A117S+L149R+T211H+S214Q+V258P+A276S+H358D+A364G+S388K+D470T+L530R;

V45L+A79C+L149R+T215V+V258P+A276S+A364G+S388K;

V45L+A117S+L149R+S214Q+V258P+A276S+H358D+A364G+S388K+D470T+R483V+V500T;

V45L+A79C+L149R+V258P+A276S+A286S+A364G+S388K;

V45L+A79C+L149R+H219V+V258P+A276S+A364G+S388K;

V45L+A79C+L149R+A170S+V258P+A276S+A364G+S388K;

V45L+D113T+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A79C+L149R+V258P+A276S+A364G+S388K;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+T215I+V258P+A276S+H358D+A364G+S388K+D470T+R483V;

V45L+A117S+L149R+S214Q+H219I+V258P+A276S+H358D+A364G+S388K+D470T+R483V;

V45L+A117S+L149R+S214Q+V258P+A276S+H358D+A364G+S388K+D470T+L478N;

V45L+A117S+L149R+S214Q+V258P+A276S+L281V+H358D+A364G+S388K+D470T;

V45L+A117S+L149R+S214E+V258P+A276S+H358D+A364G+S388K+D470T;

V45L+T70R+A117S+L149R+S214Q+V258P+A276S+L281V+H358D+A364G+S388K+G394A+C468L+D470T+R483V;

V45L+A117S+L149R+S214Q+V258P+A276S+H358D+A364G+Q385C+S388K+D470T+L530R;

V45L+A117S+L149R+S214Q+V258P+A276S+H358D+A364G+T366I+S388K+D470T;

V45L+A117S+L149R+S214Q+V258P+A276S+H358D+A364G+S388K+D470T+S473G;

V45L+A117S+L149R+S214Q+V258P+A276S+H358D+A364G+S388K+D470T+L478P;

V45L+A117S+L149R+S214Q+V258P+A276S+H358D+A364G+S388K+G394D+D470T;

V45L+A117S+L149R+S214Q+V258P+A276S+A364G+Q384A+S388K+D470T+R483V;

D11E+V45L+A79C+L149R+V258P+A276S+A364G+S388K;

V45L+A117S+L149R+S214Q+V258P+A276S+H358D+A364G+S388K+D470T+R483V+L530R;

V45L+A117S+L149R+S214Q+V258P+A276S+H358D+A364G+S388K+D470T+N529T;

V45L+A117S+L149R+S214Q+V258P+A276S+H358D+A364G+S388K+G394Q+D470T;

V45L+A117S+L149R+G187L+S214Q+V258P+A276S+H358D+A364G+S388K+D470T;

V45L+A79C+L149R+V258P+A276S+A286E+A364G+S388K;

V45L+A117S+L149R+S214Q+V258P+A276S+H358D+A364G+S388K+D470T+R483V+G503L;

V45L+A117S+L149R+S214Q+V258P+A276S+Q314A+H358D+A364G+S388K+D470T;

V45L+A117S+L149R+S214Q+V258P+A276S+V350L+H358D+A364G+S388K+D470T;

V45L+A117S+L149R+G187K+S214Q+V258P+A276S+H358D+A364G+S388K+D470T;

V45L+A117S+L149R+S214Q+W250Y+V258P+A276S+H358D+A364G+S388K+D470T;

V45L+A117S+L149R+S214Q+V258P+A276S+A286E+H358D+A364G+S388K+D470T;

V45L+A117S+L149R+S214Q+V258P+A276S+H358D+A364G+Q384A+S388K+D470T;

V45L+A117S+L149R+S214Q+V258P+A276S+H358D+A364G+S388R+D470T;

V45L+A117S+L149R+S214Q+V258P+A276S+H358D+A364G+S388K+D470T+S528H, V45L+A117S+L149R+S214Q+T215V+V258P+A276S+H358D+A364G+S388K+D470T+R483V;

V45L+A117S+L149R+S214Q+V258P+A276S+H358D+A364G+S388K+D470T+Q496R;

V45L+A117S+L149R+N163H+S214Q+V258P+A276S+H358D+A364G+S388K+D470T;

V45L+A117S+L149R+S214Q+V258P+A276S+S282N+H358D+A364G+S388K+D470T;

V45L+A117S+L149R+S214Q+V258P+A276Q+H358D+A364G+S388K+D470T;

D11E+V45L+A117S+L149R+S214Q+V258P+A276S+H358D+A364G+S388K+D470T;

V45L+A117S+L149R+S214Q+V258P+A276S+H358D+A364G+T366V+S388K+D470T;

V45L+A117S+L149R+S214Q+V258P+A276S+H358D+A364G+S388K+D470T+S473K;

V45L+A79C+L149R+T180Q+V258P+A276S+A364G+S388K;

V45L+A117S+L149R+S214Q+V258P+A276S+H358D+A364G+S388K+D470T+S481A+R483V;

V45L+A117S+L149R+S214Q+V258P+A276S+H358D+A364G+S388R+D470T+V45L+A117S+L149R+S214Q+V258P+A276S+Q314A+A364G+S388K+D470T+R483V;

V45L+S92D+A117S+L149R+S214Q+V258P+A276S+H358D+A364G+S388K+D470T;

V45L+A117S+L149R+S214Q+V258P+N260H+A276S+H358D+A364G+S388K+D470T;

V45L+A117S+L149R+G187S+S214Q+V258P+A276S+H358D+A364G+S388K+D470T;

V45L+A117S+L149R+S214Q+V258P+A276S+H358D+A364G+S388K+D470T+T477K;

V45L+A117S+L149R+S214Q+V258P+A276S+H358D+A364G+S388K+D470T+N474G;

V45L+A117S+L149R+S214Q+V258P+A276S+H358D+A364G+S388K+N395D+D470T;

V45L+A117S+L149R+S214Q+V258P+A276S+H358D+A364G+S388K+D470T+S501N;

V45L+A117S+N127D+L149R+S214Q+V258P+A276S+H358D+A364G+S388K+D470T;

V45L+A117S+L149R+S214Q+V258P+A276S+H358D+A364G+S388K+V393R+D470T;

V45L+A117S+L149R+S173Y+S214Q+V258P+A276S+H358D+A364G+S388K+D470T;

V45L+A117S+L149R+S214Q+V258P+A276S+H358D+A364G+S388H+D470T;

V45L+A117S+L149R+S214Q+T215V+V258P+A276S+H358D+A364G+S388K+D470T+L530R;

V45L+A117S+L149R+G187S+S214Q+V258P+A276S+H358D+A364G+S388K+D470T+L530R;

V45L+A117S+L149R+T211H+S214Q+V258P+A276S+H358D+A364G+S388K+D470T+R483V;

V45L+A117S+L149R+S214Q+V258P+A276S+H358D+A364G+S388K+D470T+T480N;

V45L+A117S+L149R+N168G+S214Q+V258P+A276S+H358D+A364G+S388K+D470T;

V45L+A79C+L149R+H219I+V258P+A276S+A364G+S388K;

V45L+L149R+S214E+V258P+A276S+H358D+A364G+D470T;

V45L+Q61K+A117S+L149R+S214Q+V258P+A276S+A364G+S388K+D470T+V45L+D113T+A117S+L149R+S214Q+V258P+A276S+H358D+A364G+S388K+D470T;

V45L+A117S+L149R+S214Q+V258P+A276S+A364G+T366V+S388K+D470T+R483V;

V45L+A117S+L149R+S214Q+V258P+A276S+H358T+A364G+S388K+D470T+R483V;

V45L+A117S+L149R+S214Q+V258P+A276S+A364G+S388K+V393R+D470T+R483V;

V45N+A79C+L149R+V258P+A276S+A364G+S388K;

V45L+A117S+L149R+S214Q+V258P+A276S+H358D+A364G+S388K+D470T+Q496Q;

V45L+A117S+L149R+S214Q+V258P+A276S+A364G+S388K+G394Q+D470T+R483V;

V45L+A79C+L149R+V258P+A276S+V350L+A364G+S388K;

V45L+L149R+S214E+V258P+A276S+H358D+A364G+D470T;

V45L+A117S+L149R+S214Q+V258P+A276S+A364G+T366I+S388K+D470T+R483V;

V45L+A117S+L149R+S214Q+V258P+A276S+H358D+A364G+S388K+D470T+R483V+G526T;

V45L+A117S+L149R+S214Q+V258P+A276S+H358D+A364G+S388K+D470T+L484S;

V45L+A79C+A117S+L149R+S214Q+V258P+A276S+H358D+A364G+S388K;

V45L+A117S+L149R+S214Q+V258P+A276S+H358D+A364G+S388K+D470T+A499L;

V45L+A117S+L149R+S214Q+V258P+A276S+H358T+A364G+S388K+D470T+L530R;

V45L+A79C+D113T+L149R+V258P+A276S+A364G+S388K;

V45L+A117S+L149R+S214Q+V258P+A276S+H358D+A364G+S388K+D470T+R483V+D525L;

V45L+A117S+L149R+S214Q+V258P+A276S+H358D+A364G+S388K+D470T+V498I;

V45L+A117S+L149R+S214Q+V258P+A276S+H358D+A364G+S388L+D470T;

V45L+T70R+A117S+L149R+S214Q+V258P+A276S+A364G+S388K+D470T+R483V;

V45L+A117S+L149R+S214Q+V258P+A276S+H358D+A364G+S388K+D470T+S528H+L530R;

V45L+A117S+L149R+S214Q+V258P+A276S+A364G+S388K+D470T+N476D+R483V;

D9E+V45L+A117S+L149R+S214Q+V258P+A276S+A364G+S388K+D470T+R483V;

V45L+A117S+L149R+S214Q+V258P+A276S+H358D+A364G+S388K+D470T+S501E;

V45L+A117S+L149R+S214Q+V258P+A276S+H358D+A364G+S388K+D470T+S481A;

V45L+A117S+L149R+S214Q+H219V+V258P+A276S+H358D+A364G+S388K+D470T;

V45L+A117S+L149R+S214Q+V258P+A276S+H358D+A364G+S388K+D470T+S481S;

V45L+A117S+L149R+G187K+S214Q+V258P+A276S+A364G+S388K+D470T+R483V;

V45L+A117S+L149R+S214Q+V258P+A276S+A364G+S388K+N395D+D470T+R483V;

V45L+A117S+L149R+S214Q+V258P+A276S+H358D+A364G+S388K+I469T+D470T+R483V;

V45L+A79C+N127D+L149R+V258P+A276S+A364G+S388K;

D9E+V45L+A79C+L149R+V258P+A276S+A364G+S388K;

V45L+L149R+S214E+V258P+A276S+H358D+A364G+D470T;

V45L+A117S+L149R+G187L+S214Q+V258P+A276S+A364G+S388K+D470T+R483V;

V45L+A117S+L149R+S214Q+V258P+A276S+H358D+A364G+S388K+D470T+N529T+L530R;

V45L+A79C+L149R+W250Y+V258P+A276S+A364G+S388K;

V45L+A117S+L149R+S214Q+V258P+A276S+H358D+A364G+S388K+D470T+V497P;

V45L+A117S+L149R+S214Q+V258P+A276S+H358D+A364G+S388K+D470T+N527H;

V45L+A117S+L149R+S214Q+V258P+A276S+A364G+S388K+D470T+R483V+Q496Q;

V45L+A117S+L149R+N168G+S214Q+V258P+A276S+A364G+S388K+D470T+R483V;

V45L+A117S+L149R+S214Q+V258P+A276S+A364G+S388K+D470T+N476N+V45L+A117S+L149R+S214Q+V258P+A276S+A364G+S388K+D470T+T477V+R483V;

V45L+A117S+L149R+S214Q+V258P+A276S+V350L+A364G+S388K+D470T+R483V;

V45L+A117S+L149R+S214Q+V258P+A276S+A364G+S388K+D470T+L478N+R483V;

V45L+A117S+L149R+S214E+V258P+A276S+A364G+S388K+D470T+R483V;

M28P+V45L+A117S+L149R+S214Q+V258P+A276S+H358D+A364G+S388K+D470T;

V45L+A117S+L149R+S214Q+V258P+A276S+A364G+S388K+V393H+D470T+R483V;

V45L+Q61R+A117S+L149R+S214Q+V258P+A276S+A364G+S388K+D470T+R483V;

V45L+A117S+L149R+S214Q+V258P+A276S+A364G+S388K+D470T+R483V+V497P;

V45L+A117S+L149R+S214Q+V258P+A276S+A364G+S388K+D470T+R483V+V498I;

V45L+A117S+L149R+S214Q+V258P+A276S+S282N+H358D+A364G+S388K+D470T+L530R;

V45L+T70R+A117S+L149R+S214Q+V258P+A276S+L281V+H358D+A364G+C375V+S388K+G394A+D470T+R483V;

V45L+A117S+L149R+S214Q+V258P+A276S+A286E+A364G+S388K+D470T+R483V;

V45L+A117S+L149R+S214Q+V258P+A276S+A364G+S388R+D470T+R483V;

V45L+A117S+L149R+S214Q+V258P+A276S+A364G+S388K+D470A+R483V;

V45L+A117S+L149R+S214Q+V258P+A276S+A364G+S388K+D470T+T477K+R483V;

V45L+A117S+L149R+S214Q+V258P+A276S+A364G+S388K+D470T+R483V+Q496E;

V45L+A117S+L149R+S214Q+V258P+A276S+A364G+S388K+D470T+R483V+Q496R;

V45L+A117S+L149R+S214Q+V258P+A276S+A364G+S388K+D470T+R483V+A499L;

V45L+L149R+S214E+V258P+A276S+H358D+A364G+D470T;

V45L+L149R+S214E+V258P+A276S+H358D+A364G+D470T;

V45L+A117S+L149R+S214Q+T215I+V258P+A276S+H358D+A364G+S388K+V45L+T70R+A117S+L149R+S214Q+V258P+A276S+L281V+H358D+A364G+S388K+G394A+C468S+D470T+R483V;

V45L+A117S+L149R+S214Q+V258P+A276S+A364G+S388K+Y391V+D470T+R483V;

V45L+A117S+L149R+S214Q+V258P+A276Q+A364G+S388K+D470T+R483V;

V45L+A117S+L149R+S214Q+V258P+A276S+H358D+A364G+S388K+D470T+G503L+L530R;

V45L+A117S+L149R+S214Q+H219I+V258P+A276S+H358D+A364G+S388K+D470T+L530R;

V45L+A117S+L149R+S214Q+V258P+A276S+H358D+A364G+S388K+D470T+N476D;

V45L+A117S+L149R+S214Q+V258P+A276S+H358D+A364G+S388K+V393H+D470T;

V45L+A117S+L149R+S214Q+V258P+A276S+A364G+S388K+D470T+R483V;

V45L+A117S+L149R+S214Q+V258P+A276S+A286S+A364G+S388K+D470T+R483V;

V45L+A117S+L149R+S214Q+V258P+A276S+A364G+S388K+D470T+R483V+S501E;

V45L+A117S+L149R+S214Q+V258P+A276S+A364G+S388K+D470T+R483V+N529T;

V45L+A117S+L149R+S214Q+V258P+A276S+A364G+S388K+D470T+R483V+S501N;

V45L+A117S+L149R+S214Q+V258P+A276S+H358D+A364G+S388K+D470T;

V45L+A117S+L149R+S214Q+V258P+A276S+A364G+S388K+D470T+S481S+R483V;

V45L+A117S+L149R+S214Q+V258P+A276S+A364G+S388K+D470T+L478P+R483V;

D11E+V45L+A117S+L149R+S214Q+V258P+A276S+A364G+S388K+D470T+R483V;

V45L+A117S+L149R+S214Q+V258P+A276S+H358D+A364G+S388K+D470T+T477V;

V45L+A117S+L149R+S214Q+V258P+A276S+H358D+A364G+S388K+D470T+V497P+L530R;

V45L+A117S+L149R+T211H+S214Q+V258P+A276S+H358D+A364G+S388K+D470T;

V45L+A117S+L149R+S214Y+V258P+A276S+H358D+A364G+S388K+D470T+V45L+A117S+L149R+S214Q+W250Y+V258P+A276S+A364G+S388K+D470T+R483V;

V45L+S92D+A117S+L149R+S214Q+V258P+A276S+A364G+S388K+D470T+R483V;

V45L+A117S+L149R+S214Q+V258P+A276S+A364G+S388K+D470T+R483V+N527H;

V45N+A117S+L149R+S214Q+V258P+A276S+A364G+S388K+D470T+R483V;

V45L+A117S+L149R+S214Q+V258P+A276S+H358D+A364G+Q385C+S388K+D470T;

V45L+A117S+L149R+S173Y+S214Q+V258P+A276S+A364G+S388K+D470T+R483V;

V45L+A117S+L149R+S214Q+V258P+A276S+A364G+S388H+D470T+R483V;

V45L+A117S+L149R+S214Q+V258P+A276S+A364G+S388K+S389H+D470T+R483V;

V45L+A117S+L149R+S214Q+H219V+V258P+A276S+A364G+S388K+D470T+R483V;

V45L+A79C+A117S+L149R+S214Q+V258P+A276S+A364G+S388K+D470T;

V45L+A117S+L149R+S214Q+T215I+V258P+A276S+H358D+A364G+S388K+D470T+L530R;

V45L+A117S+L149R+S214Q+V258P+A276S+H358D+A364G+S388K+D470T+Q496E;

V45L+A117S+L149R+A170S+S214Q+V258P+A276S+H358D+A364G+S388K+D470T;

V45L+A117S+L149R+S214Q+V258P+A276S+H358D+A364G+S388K+S389V+D470T+R483V;

V45L+A117S+L149R+S214Q+V258P+A276S+H358D+A364G+S388K+S389V+D470T+L530R;

V45L+A117S+L149R+S214Q+V258P+A276S+H358D+A364G+S388A+D470T;

V45L+A117S+L149R+S214Q+V258P+A276S+H358D+A364G+S388K+S389V+D470T;

V45L+T70R+A117S+L149R+S214Q+V258P+A276S+H358D+A364G+S388K+D470T;

V45L+A117S+L149R+S214Q+H219I+V258P+A276S+A364G+S388K+D470T+R483V;

V45L+A79C+L149R+N163H+V258P+A276S+A364G+S388K;

V45L+A117S+L149R+S214Q+V258P+A276S+A364G+S388K+D470T+R483V+A499Y;

V45L+A117S+L149R+S214Q+V258P+A276S+L281V+A364G+S388K+D470T+R483V;

V45L+A117S+L149R+S214Q+V258P+A276S+A364G+S388K+D470T+R483V+V500T;

V45L+A117S+L149R+T180Q+S214Q+V258P+A276S+H358D+A364G+S388K+D470T;

V45L+A117S+L149R+S214Q+V258P+A276S+H358D+A364G+S388K+D470T+V500V;

V45L+A117S+L149R+S214Q+V258P+A276S+H358D+A364G+S388K+D470T+G526T;

V45L+C60L+T70R+A117S+L149R+S214Q+V258P+A276S+L281V+H358D+A364G+S388K+G394A+D470T+R483V;

V45L+C69L+T70R+A117S+L149R+S214Q+V258P+A276S+L281V+H358D+A364G+S388K+G394A+D470T+R483V;

V45L+A117S+L149R+S214Q+V258P+A276S+A364G+S388A+D470T+R483V;

V45L+A117S+L149R+S214Q+V258P+A276S+A286S+H358D+A364G+S388K+D470T;

V45L+A117S+L149R+S214Q+V258P+A276S+H358D+A364G+S388K+D470T+R483V+V497D;

V45L+A117S+L149R+S214Q+V258P+A276S+H358D+A364G+S388K+D470A+L530R;

V45L+A117S+L149R+S214Q+V258P+A276S+A364G+S388K+D470T+R483V+D525L;

V45L+A117S+L149R+S214Q+V258P+A276S+H358D+A364G+S388K+D470T+N476N;

V45L+A117S+L149R+S214Q+V258P+A276S+L281V+A364G+S388K+D470T;

V45L+A117S+L149R+S214Q+V258P+A276S+A364G+Q384A+S388K+D470T;

V45L+A117S+L149R+S214Q+V258P+A276S+A286E+A364G+S388K+D470T;

V45L+A117S+L149R+S214Q+V258P+A276S+A364G+S388K+D470T+T477K;

V45L+A117S+L149R+S214Q+V258P+A276S+H358D+A364G+S388K+S389H+D470T;

V45L+A117S+L149R+S214Y+V258P+A276S+H358D+A364G+S388K+D470T+L530R;

V45L+A117S+L149R+S214Y+V258P+A276S+H358D+A364G+S388K+D470T;

V45L+A117S+L149R+S214Q+V258P+A276S+A364G+S388K+G394A+D470T;

V45L+A117S+L149R+S214Q+V258P+A276S+A364G+S388K+D470T+N476D;

V45L+A117S+L149R+S214Q+V258P+A276S+H358D+A364G+S388K+D470T+L478N+R483V;

V45L+C60A+T70R+A117S+L149R+S214Q+V258P+A276S+L281V+H358D+A364G+S388K+G394A+D470T+R483V;

V45L+A117S+L149R+S214Q+V258P+A276S+A364G+S388K+V393H+D470T;

V45L+A117S+L149R+S214Q+V258P+A276S+A364G+S388K+N395D+D470T;

A25W+V45L+A117S+L149R+S214Q+V258P+A276S+H358D+A364G+S388K+D470T;

V45L+T70R+A117S+L149R+S214Q+C243L+V258P+A276S+L281V+H358D+A364G+S388K+G394A+D470T+R483V;

V45L+A117S+L149R+S214Q+V258P+A276S+H358T+A364G+S388K+D470T;

V45L+A117S+L149R+S214Q+V258P+A276S+A364G+S388K+D470T+L478N;

V45L+C60S+T70R+A117S+L149R+S214Q+V258P+A276S+L281V+H358D+A364G+S388K+G394A+D470T+R483V;

V45L+A117S+L149R+S214Q+W250Y+V258P+A276S+A364G+S388K+D470T;

V45L+A117S+L149R+S214Q+T215V+V258P+A276S+A364G+S388K+D470T+R483V;

V45L+Q61R+A117S+L149R+S214Q+V258P+A276S+H358D+A364G+S388K+D470T;

V45L+A117S+L149R+G187L+S214Q+V258P+A276S+A364G+S388K+D470T;

D9E+V45L+A117S+L149R+S214Q+V258P+A276S+A364G+S388K+D470T;

V45L+A117S+L149R+S214Q+V258P+A276S+H358D+A364G;

V45L+C69A+T70R+A117S+L149R+S214Q+V258P+A276S+L281V+H358D+A364G+S388K+G394A+D470T+R483V;

V45L+T70R+A117S+L149R+C161S+S214Q+V258P+A276S+L281V+H358D+A364G+S388K+G394A+D470T+R483V;

V45L+A117S+L149R+S214Q+H219I+V258P+A276S+H358D+A364G+S388K+D470T;

V45L+A117S+L149R+S214Q+V258P+A276S+S282N+A364G+S388K+D470T;

V45L+A117S+L149R+T211H+S214Q+V258P+A276S+A364G+S388K+D470T+R483V;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

D11E+V45L+A117S+L149R+S214Q+V258P+A276S+A364G+S388K+D470T;

V45L+A117S+L149R+S214Q+V258P+A276S+A364G+S388K+D470T+N476N;

V45L+A117S+L149R+S214Q+V258P+A276S+A364G+S388K+G394D+D470T;

V45L+Q61K+A117S+L149R+S214Q+V258P+A276S+A364G+S388K+D470T;

V45L+A117S+L149R+S214Q+V258P+A276S+H358D+A364G+S388K+D470T+G503L;

V45L+A117S+L149R+S214Q+V258P+A276S+A286S+A364G+S388K+D470T;

V45L+A117S+L149R+S214Q+V258P+A276S+H358D+A364G+S388K+D470A;

V45L+A117S+L149R+S214Q+V258P+A276S+A364G+S388K+G394Q+D470T;

V45L+A117S+L149R+S214Q+V258P+A276S+A364G+S388R+D470T;

V45L+A117S+L149R+S214Q+V258P+A276S+A364G+S388K+D470T+L478P;

V45L+A117S+L149R+S214Q+V258P+A276S+V350L+A364G+S388K+D470T;

V45L+D113T+A117S+L149R+S214Q+V258P+A276S+A364G+S388K+D470T;

V45L+A117S+L149R+T180Q+S214Q+V258P+A276S+A364G+S388K+D470T;

V45L+C69V+T70R+A117S+L149R+S214Q+V258P+A276S+L281V+H358D+A364G+S388K+G394A+D470T+R483V;

V45L+A117S+L149R+S214Q+V258P+A276S+H358D+A364G+S388K+D470T+D525L;

V45L+A117S+L149R+S214Q+V258P+A276S+A364G+S388K+D470T;

V45L+A117S+L149R+S214Q+V258P+A276S+H358D+A364G+S388K+D470T+V500T;

V45L+A117S+L149R+S214Q+V258P+A276S+A364G+S388K+S389H+D470T;

V45L+A117S+L149R+S214Q+V258P+A276S+A364G+S388K+V393R+D470T;

V45L+A117S+L149R+S214Q+V258P+A276S+A364G+S388K+D470T+Q496R;

V45L+A117S+N127D+L149R+S214Q+V258P+A276S+A364G+S388K+D470T;

V45L+A117S+L149R+S214Q+V258P+A276S+A364G+S388K+D470T+A499Y;

V45L+A117S+L149R+S214Q+T215V+V258P+A276S+H358D+A364G+S388K+D470T;

V45L+A117S+L149R+S214Q+V258P+A276S+H358D+A364G+S388K+D470T+T480L;

V45L+A117S+L149R+S214Q+V258P+A276S+A364G+S388L+D470T;

V45L+A117S+L149R+S173Y+S214Q+V258P+A276S+A364G+S388K+D470T;

V45L+S92D+A117S+L149R+S214Q+V258P+A276S+A364G+S388K+D470T;

V45L+A117S+L149R+S214Q+V258P+A276S+A364G+S388K+S389V+D470T+R483V;

V45L+A117S+L149R+S214Q+V258P+A276S+A364G+S388K+D470T+N529T;

V45L+C60V+T70R+A117S+L149R+S214Q+V258P+A276S+L281V+H358D+A364G+S388K+G394A+D470T+R483V;

V45L+A117S+L149R+S214Q+V258P+A276S+A364G+S388K+D470T+R483V+V497E;

V45L+A117S+L149R+S214Q+V258P+A276S+H358D+A364G+S388K+L478N;

V45L+T70R+A117S+L149R+S214Q+V258P+A276S+A364G+S388K+D470T;

M28P+V45L+A117S+L149R+S214Q+V258P+A276S+A364G+S388K+D470T;

V45L+A117S+L149R+S214Q+V258P+A276Q+A364G+S388K+D470T;

V45L+A117S+L149R+S214Q+V258P+A276S+H358D+A364G+S388K+D470V+R483V;

V45L+A117S+L149R+S214Y+V258P+A276S+A364G+S388K+D470T+R483V;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

V45L+Q61R+A117S+L149R+S214Q+V258P+A276S+A364G+S388K+D470T;

V45L+A117S+L149R+S214Q+T215I+V258P+A276S+A364G+S388K+D470T;

V45L+A117S+L149R+S214Q+V258P+A276S+A364G+S388K+D470T+Q496E;

V45L+A117S+L149R+G187K+S214Q+V258P+A276S+A364G+S388K+D470T;

V45L+A117S+L149R+S214Q+V258P+A276S+A364G+S388K+D470T+W495I;

V45L+A117S+L149R+S214Q+V258P+A276S+A364G+S388K+D470T+V498I;

V45L+A117S+L149R+S214Q+V258P+A276S+H358D+A364G+S388K+D470K+R483V;

V45L+A117S+L149R+S214Q+V258P+A276S+A364G+S388K+D470T+V500V;

D11E+V45L+A117S+L149R+S214Q+V258P+A276S+H358D+A364G+S388K;

V45L+A117S+L149R+A170S+S214Q+V258P+A276S+A364G+S388K+D470T;

V45L+A117S+L149R+S214Q+V258P+A276S+A364G+S388K+Y391V+D470T;

V45L+A117S+L149R+S214Q+V258P+A276S+Q314A+A364G+S388K+D470T;

V45L+A117S+L149R+S214Q+V258P+A276S+A364G+S388K+D470T+T477V;

A25W+V45L+A79C+L149R+S214E+V258P+A276S+A364G;

V45L+A117S+L149R+S214Q+V258P+A276S+H358D+A364G+S388K+G394Q;

V45L+A117S+L149R+S214Q+H219V+V258P+A276S+A364G+S388K+D470T;

V45L+A117S+L149R+S214Q+V258P+A276S+A364G+S388K+D470T+Q496Q;

V45L+T70R+A117S+L149R+S214Q+V258P+A276S+L281V+H358D+A364G+C375A+S388K+G394A+D470T+R483V;

V45L+A117S+L149R+D192E+S214Q+V258P+A276S+H358D+A364G;

V45L+A117S+L149R+S214Q+V258P+A276S+H358D+A364G+S388K+D470T+A472L;

V45L+A117S+L149R+S214Q+V258P+A276S+H358D+A364G+S388K+A499Y;

V45L+A117S+L149R+S214Q+T215V+V258P+A276S+A364G+S388K+D470T;

V45L+A117S+L149R+S214Q+V258P+A276S+A364G+Q385C+S388K+D470T;

V45L+A117S+L149R+S214Q+V258P+A276S+A364G+S388A+D470T;

V45L+A117S+L149R+S214Q+V258P+A276S+A364G+S388H+D470T;

V45L+A117S+L149R+S214Q+V258P+A276S+A364G+S388K+D470T+T480N;

V45L+A117S+L149R+S214Q+V258P+A276S+A364G+S388K+D470T+V497P;

V45L+A117S+L149R+S214Q+V258P+A276S+A364G+S388K+D470T+A499L;

V45L+A117S+L149R+S214Q+V258P+A276S+A364G+S388K+D470T+S501N;

V45L+A117S+L149R+S214Q+V258P+A276S+A364G+D470T;

V45L+A117S+L149R+S214Q+V258P+A276S+H358D+A364G+Q384A+S388K;

V45L+T70R+A117S+L149R+S214Q+V258P+A276S+L281V+H358D+A364G+C375S+S388K+G394A+D470T+R483V;

V45L+A117S+L149R+S214Q+V258P+A276S+A364G+S388L+D470T+R483V;

V45L+A79C+S92D+L149R+V258P+A276S+A364G+S388K;

V45L+T70R+A117S+L149R+S214Q+V258P+A276S+L281V+H358D+A364G+S388K+G394A+D470T+R483V+C540A;

V45N+A117S+L149R+S214Q+V258P+A276S+A364G+S388K+D470T;

V45L+A117S+L149R+S214Q+V258P+A276S+H358D+A364G+S388K+G394A;

V45L+C69S+T70R+A117S+L149R+S214Q+V258P+A276S+L281V+H358D+A364G+S388K+G394A+D470T+R483V;

V45L+A117S+L149R+S214Q+V258P+A276S+H358D+A364G+S388K+V498I;

V45L+A117S+L149R+S214Q+V258P+A276S+H358D+A364G;

V45L+A117S+L149R+S214Q+H219I+V258P+A276S+A364G+S388K+D470T;

V45L+A117S+L149R+N163H+S214Q+V258P+A276S+A364G+S388K+D470T;

V45L+L149R+S214Q+V258P+A276S+H358D+A364G;

V45L+A117S+L149R+S214Q+V258P+A276S+Q314A+H358D+A364G+S388K;

V45L+A117S+L149R+S173Y+S214Q+V258P+A276S+H358D+A364G+S388K;

V45L+A117S+L149R+S214E+V258P+A276S+H358D+A364G+S388K;

V45L+A117S+L149R+S214Q+V258P+A276S+H358D+A364G+S388K+G394D;

A25W+V45L+A79C+L149R+V258P+A276S+A364G;

V45L+L149R+S214Q+V258P+A276S+A286E+A364G+S388K;

V45L+Q61R+A117S+L149R+S214Q+V258P+A276S+H358D+A364G+S388K;

V45L+A117S+L149R+S214Q+V258P+A276S+H358D+A364G+T366V+S388K;

V45L+A117S+L149R+D192E+S214Q+V258P+A276S+H358D+A364G;

V45L+A117S+L149R+T180Q+S214Q+V258P+A276S+H358D+A364G+S388K;

V45L+A117S+L149R+S214Q+T215I+V258P+A276S+H358D+A364G+S388K;

V45L+A117S+L149R+S214Q+W250Y+V258P+A276S+H358D+A364G+S388K;

V45L+L149R+S214Q+T215I+V258P+A276S+A364G+S388K;

V45L+A117S+L149R+S214Q+V258P+A276S+H358D+A364G+S388K+N476D;

V45L+D113T+A117S+L149R+S214Q+V258P+A276S+A364G+S388K+D470T+R483V;

V45L+A117S+L149R+S214Q+T215K+V258P+A276S+A364G+S388K+D470T+R483V;

V45L+A117S+L149R+S214Q+V258P+A276S+H358D+A364G+S388K;

V45L+A117S+L149R+S214Q+V258P+A276S+H358D+A364G+S388K+T477V;

V45L+A117S+L149R+S214Q+V258P+A276S+H358D+A364G+S388K+L478P;

V45L+A117S+L149R+S214Q+V258P+A276S+H358D+A364G+S388K+S481A;

A25W+V45L+L149R+S214Q+V258P+A276S+A364G+S388K;

V45L+A117S+L149R+T211H+S214Q+V258P+A276S+A364G+S388K+D470T;

V45L+A117S+L149R+S214Q+T215K+V258P+A276S+H358D+A364G+S388K+D470T+R483V;

V45L+A79C+L149R+V258P+A276S+L281V+A364G;

V45L+A117S+L149R+S214Q+V258P+A276S+A364G+S388K+R483V;

V45L+A117S+L149R+S214Q+V258P+A276S+H358D+A364G+S388K+V500T;

V45L+A117S+L149R+S214Q+V258P+A276S+V350L+H358D+A364G+S388K;

V45L+A117S+L149R+G187K+S214Q+V258P+A276S+H358D+A364G+S388K;

V45L+T70R+A117S+L149R+S214Q+V258P+A276S+L281V+H358D+A364G+C375L+S388K+G394A+D470T+R483V;

V45L+L149R+A170S+S214Q+V258P+A276S+A364G+S388K;

V45L+A117S+L149R+S214E+V258P+A276S+A364G+S388K+D470T;

V45L+A117S+L149R+S214Q+V258P+A276S+A364G+S388K+D470V+R483V;

V45L+A117S+L149R+S214Q+V258P+A276S+H358D+A364G+S388K+V500V;

V45L+Q61K+L149R+S214Q+V258P+A276S+A364G+S388K;

V45L+L149R+N168G+S214Q+V258P+A276S+A364G+S388K;

V45L+A117S+L149R+N163H+S214Q+V258P+A276S+H358D+A364G+S388K;

V45L+A79C+D95D+L149R+V258P+A276S+A364G+S388K;

V45L+Q77E+A79C+L149R+V258P+A276S+A364G+S388K;

V45L+A117S+L149R+S214Q+V258P+A276S+A364G+S388K+D470T+R483V+V497W;

V45L+L149R+S214Q+V258P+A276S+S282N+A364G+S388K;

V45L+A117S+L149R+S214Q+V258P+A276S+H358D+A364G+S388R;

V45L+A117S+L149R+S214Q+V258P+A276S+H358D+A364G+S388K+S481S;

V45L+A117S+L149R+S214Q+V258P+A276S+A364G+S388K+D470T+V500T;

V45L+T70R+A117S+L149R+S214Q+V258P+A276S+L281V+H358D+A364G+S388K+G394A+D470T+R483V+C540L;

V45L+T70R+A117S+L149R+S214Q+V258P+A276S+L281V+H358D+A364G+S388K+G394A+D470T+R483V+C522S;

V45L+A117S+L149R+S214Q+V258P+A276S+H358D+A364G+S388K+W495S;

V45L+L149R+S214Q+V258P+A276S+Q314A+A364G+S388K;

V45L+D113T+A117S+L149R+S214Q+V258P+A276S+H358D+A364G+S388K;

V45L+A117S+L149R+S214Q+V258P+A276S+H358D+A364G+S388K+D470T+R483V+V497W;

V45L+T70R+A117S+L149R+S214Q+V258P+A276S+L281V+H358D+A364G+S388K+G394A+D470T+R483V+C540S;

V45L+T70R+A117S+L149R+S214Q+V258P+A276S+L281V+H358D+A364G+S388K+G394A+C396S+D470T+R483V;

V45L+T70R+L149R+S214Q+V258P+A276S+A364G+S388K;

V45L+A117S+L149R+S214Q+V258P+A276S+H358D+A364G+S388K+N474G;

V45L+A117S+L149R+S214Q+V258P+A276S+S282N+H358D+A364G+S388K;

V45L+A117S+L149R+S214Q+V258P+A276S+H358D+A364G+S388K+Q496Q;

V45L+A117S+L149R+S214Q+V258P+A276S+A364G+S388K+S389V+D470T;

V45L+T70R+A117S+L149R+S214Q+V258P+A276S+L281V+H358D+A364G+S388K+G394A+C396A+D470T+R483V;

V45L+A117S+L149R+S214Q+V258P+A276S+H358D+A364G+S388K+S501E;

A25W+V45L+L149R+S214E+V258P+A276S+A364G;

V45L+L149R+S214Q+W250Y+V258P+A276S+A364G+S388K;

V45L+L149R+G187K+S214Q+V258P+A276S+A364G+S388K;

V45L+A117S+L149R+S214Q+V258P+A276S+H358D+A364G+S388K+N476N;

V45L+A117S+L149R+S214Q+V258P+A276S+H358D+A364G+S388K+S473K;

V45L+A117S+L149R+G187L+S214Q+V258P+A276S+H358D+A364G+S388K;

C40L+V45L+T70R+A117S+L149R+S214Q+V258P+A276S+L281V+H358D+A364G+S388K+G394A+D470T+R483V;

V45L+A117S+L149R+S214Q+V258P+A276S+H358D+A364G+S388K+Q496E;

A25W+V45L+A117S+L149R+S214Q+V258P+A276S+H358D+A364G+S388K;

V45L+L149R+V258P+A276S+L281V+H358T+A364G+S388K;

V45L+A117S+L149R+S214Q+V258P+A276S+H358D+A364G+S388A;

V45L+A117S+L149R+N168G+S214Q+V258P+A276S+H358D+A364G+S388K;

V45L+A117S+L149R+S214Q+V258P+A276S+H358D+A364G+S388K+S389H;

M28P+V45L+L149R+S214Q+V258P+A276S+A364G+S388K;

V45L+A117S+L149R+S214Q+V258P+A276S+A364G+S388K+D470T+T480L;

V45L+A117S+L149R+S214Q+V258P+A276S+A364G+Q384A+S388K;

V45L+A93S+L149R+A252S+H334D;

C40A+V45L+T70R+A117S+L149R+S214Q+V258P+A276S+L281V+H358D+A364G+S388K+G394A+D470T+R483V;

V45L+L149R+G187L+S214Q+V258P+A276S+A364G+S388K;

V45L+A117S+L149R+S214Q+V258P+A276S+H358D+A364G+Q385C+S388K;

V45L+L149R+S214Q+V258P+A276S+A286S+A364G+S388K;

V45L+S92D+A117S+L149R+S214Q+V258P+A276S+H358D+A364G+S388K;

V45L+A117S+L149R+S214Q+V258P+A276S+H358D+A364G+S388L;

V45L+A117S+L149R+S214Q+V258P+A276S+A286E+A364G+S388K;

D9E+V45L+L149R+S214Q+V258P+A276S+A364G+S388K;

V45L+L149R+S173Y+S214Q+V258P+A276S+A364G+S388K;

V45L+A117S+L149R+A170S+S214Q+V258P+A276S+H358D+A364G+S388K;

V45L+A117S+L149R+S214Q+V258P+A276S+H358D+A364G+S388K+Y391V;

V45L+A117S+L149R+S214Q+V258P+A276S+L281V+A364G+S388K;

V45L+A117S+L149R+S214Q+V258P+A276S+A364G+S388K+G394D;

V45L+A117S+L149R+S214Q+V258P+A276S+A364G+S388K+S473G;

V45L+A79C+L149R+V258P+A276S+H358T+A364G+S388K;

V45L+L149R+S214Q+V258P+A276Q+A364G+S388K;

A25W+V45L+L149R+S214E+V258P+A276S+H358T+A364G;

V45L+A117S+L149R+S214Q+V258P+A276S+H358D+A364G+S388K+T480N;

D9E+V45L+A117S+L149R+S214Q+V258P+A276S+H358D+A364G+S388K;

V45L+A117S+L149R+S214Q+H219I+V258P+A276S+H358D+A364G+S388K;

C40S+V45L+T70R+A117S+L149R+S214Q+V258P+A276S+L281V+H358D+A364G+S388K+G394A+D470T+R483V;

C40V+V45L+T70R+A117S+L149R+S214Q+V258P+A276S+L281V+H358D+A364G+S388K+G394A+D470T+R483V;

A25W+V45L+L149R+S214E+V258P+A276S+A286E+A364G;

V45L+A117S+L149R+S214Q+V258P+A276S+A364G+S388K+G394A;

V45L+A117S+L149R+S214Q+V258P+A276S+H358D+A364G+S388K+A499L;

A25W+V45L+Q61K+L149R+S214E+V258P+A276S+A364G;

V45L+A117S+L149R+S214Q+T215V+V258P+A276S+H358D+A364G+S388K;

A25W+V45L+L149R+V258P+A276S+H358T+A364G+S388K;

V45L+A117S+L149R+S214Q+V258P+A276S+A364G+S388K+D470T+R483V+W495E A25W+V45L+A79C+L149R+V258P+A276S;

V45L+L149R+S214Q+V258P+A276S+V350L+A364G+S388K;

D11E+V45L+L149R+S214Q+V258P+A276S+A364G+S388K;

V45L+L149R+H219I+V258P+A276S+H358T+A364G+S388K;

V45L+T70R+A117S+L149R+S214Q+V258P+A276S+L281V+H358D+A364G+S388K+G394A+C396V+D470T+R483V;

V45N+L149R+S214Q+V258P+A276S+A364G+S388K;

V45L+A117S+L149R+S214Q+H219V+V258P+A276S+H358D+A364G+S388K;

V45N+A117S+L149R+S214Q+V258P+A276S+H358D+A364G+S388K;

V45L+S92D+L149R+S214Q+V258P+A276S+A364G+S388K;

V45L+L149R+V258P+A276S+H358T+A364G;

V45L+A117S+L149R+S214Q+V258P+A276S+A364G+S388K+L478P;

V45L+A117S+L149R+S214Q+V258P+A276S+A364G+S388K+S481A;

V45L+A117S+L149R+S214Q+V258P+A276S+Q314A+A364G+S388K;

A25W+V45L+L149R+T211H+S214E+V258P+A276S+A364G;

A25W+V45L+L149R+S214E+H219V+V258P+A276S+A364G;

V45L+A117S+L149R+S214Q+V258P+A276S+H358D+A364G+S388K+S389V;

V45L+A117S+L149R+S214Q+V258P+A276S+H358D+A364G+S388K+V393H;

V45L+A117S+L149R+S214Q+V258P+A276Q+H358D+A364G+S388K;

V45L+A117S+N127D+L149R+S214Q+V258P+A276S+H358D+A364G+S388K;

V45L+L149R+W250Y+V258P+A276S+H358T+A364G+S388K;

D9E+V45L+L149R+V258P+A276S+H358T+A364G+S388K;

V45L+L149R+H219V+V258P+A276S+H358T+A364G+S388K;

V45L+A117S+L149R+S214Q+V258P+A276S+H358D+A364G+S388K+A472L;

A25W+V45L+L149R+V258P+A276S+A364G;

V45L+A117S+L149R+G187K+S214Q+V258P+A276S+A364G+S388K;

V45L+A117S+L149R+S214Q+V258P+A276S+A364G+S388K+L478N;

V45L+A117S+L149R+S214Q+V258P+A276S+A364G+S388K+V393R;

V45L+A117S+L149R+S214E+V258P+A276S+A364G+S388K;

V45L+A117S+L149R+S214Q+V258P+A276S+A364G+T366V+S388K;

V45L+A117S+L149R+S214Q+V258P+A276S+H358T+A364G+S388K;

D11E+A25W+V45L+L149R+S214E+V258P+A276S+A364G;

V45L+T70R+A117S+L149R+C161V+S214Q+V258P+A276S+L281V+H358D+A364G+S388K+G394A+D470T+R483V;

V45L+A117S+L149R+T211H+S214Q+V258P+A276S+H358D+A364G+S388K;

V45L+L149R+V258P+A276S+Q314A+H358T+A364G+S388K;

D11E+V45L+A117S+L149R+S214Q+V258P+A276S+A364G+S388K;

V45L+A117S+L149R+S214Q+V258P+A276S+A364G+S388K+G394Q;

V45L+A117S+L149R+S214Q+V258P+A276S+A364G+S388K+N395D;

V45L+A117S+L149R+S214Q+V258P+A276S+A364G+S388K+V393H;

V45L+A117S+L149R+S214Q+V258P+A276S+A364G+S388K+S501E;

V45L+A117S+L149R+S214Q+V258P+A276S+A364G+S388K+S528H;

V45L+T70R+A117S+L149R+S214Q+V258P+A276S+H358D+A364G+S388K+D470T+L530R;

V45L+A117S+L149R+S214Q+V258P+A276S+A364G+S388L;

V45L+T70R+A117S+L149R+S214Q+V258P+A276S+L281V+H358D+A364G+S388K+G394A+C396L+D470T+R483V;

V45L+L149R+T180Q+S214Q+V258P+A276S+A364G+S388K;

V45L+D113T+L149R+S214Q+V258P+A276S+A364G+S388K;

V45L+A117S+L149R+S214Q+V258P+A276S+H358D+A364G+S388K+T480L;

V45L+A93S+L149R+A252S+H334D;

V45L+A93S+L149R+V234P+A252S;

V45L+A117S+L149R+S214Q+V258P+A276S+H358D+A364G+S388H;

A25W+V45L+Q61R+L149R+S214E+V258P+A276S+A364G;

V45L+A117S+L149R+S214Q+T215K+V258P+A276S+H358D+A364G+S388K+D470T;

V45L+A117S+L149R+S214Q+V258P+A276S+A364G+S388K+S389H;

V45L+A117S+L149R+S214Q+V258P+A276S+A364G+T366I+S388K;

V45L+A117S+L149R+S214Q+V258P+A276S+A364G+S388K+N474G;

A25W+V45L+L149R+S214E+V258P+A276S+L281V+A364G;

D9E+A25W+V45L+L149R+S214E+V258P+A276S+A364G;

V45L+A117S+L149R+S214Q+T215K+V258P+A276S+H358D+A364G+S388K+D470T+L530R;

V45L+A117S+L149R+S214Y+V258P+A276S+H358D+A364G+S388K;

V45L+A117S+L149R+S214Q+V258P+A276S+H358D+A364G+S388K+V393R;

V45L+Q61R+L149R+V258P+A276S+H358T+A364G+S388K;

V45L+L149R+S214Q+V258P+A276S+A364G+S388K;

V45L+D113T+L149R+V258P+A276S+H358T+A364G+S388K;

V45L+A117S+L149R+S214Q+V258P+A276S+A286S+A364G+S388K;

V45L+A117S+L149R+S214Q+V258P+A276S+A364G+S388R;

V45L+A117S+L149R+S214Q+V258P+A276S+A364G+S388K+A499Y;

V45L+A117S+L149R+S214Q+V258P+A276S+A364G+S388K+V500V;

V45L+Q61R+L149R+S214Q+V258P+A276S+A364G+S388K;

V45L+L149R+S214E+V258P+A276S+H358T+A364G;

V45L+A117S+L149R+S214Q+V258P+A276Q+A364G+S388K;

V45L+L149R+V258P+A276S+L281V+A364G;

V45L+A117S+L149R+S214Q+W250Y+V258P+A276S+A364G+S388K;

V45L+A117S+L149R+S214Q+V258P+A276S+A364G+S388K+T480N;

V45L+A117S+L149R+G187S+S214Q+V258P+A276S+A364G+S388K;

A25W+V45L+A117S+L149R+S214Q+V258P+A276S+A364G+S388K;

V45L+A117S+L149R+S214Q+V258P+A276S+A364G+S388K;

V45L+A117S+L149R+S214Q+V258P+A276S+A364G+S388K+N476N;

A25W+V45L+L149R+S214E+V258P+A276Q+A364G;

V45L+A117S+L149R+S214Q+V258P+A276S+A364G+S388K+N476D;

V45L+L149R+S214Q+V258P+A276S+L281V+A364G;

V45L+A93S+L149R+A252S+H334D+A340G;

V45L+A93S+L149R+A252S+H334D;

V45L+A93S+L149R+A252S+H334D;

V45L+L149R+A252S+H334D;

V45L+N127D+L149R+V258P+A276S+A364G+S388K;

V45L+L149R+V258P+A276S+H358T+A364G+S388K;

V45L+L149R+V258P+A276S+A364G+S388K;

D9E+V45L+A117S+L149R+S214Q+V258P+A276S+A364G+S388K;

V45L+A117S+L149R+S214Q+V258P+A276S+A364G+S388K+S481S;

V45L+L149R+N168G+V258P+A276S+A364G;

V45L+L149R+T180Q+V258P+A276S+H358T+A364G+S388K;

V45L+L149R+G187L+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

A25W+V45L+L149R+V258P+A276S+A364G+S388K;

V45L+L149R+S214Q+V258P+A276S+H358T+A364G;

V45L+L149R+S173Y+V258P+A276S+A364G;

V45L+A117S+L149R+S214Q+H219V+V258P+A276S+A364G+S388K;

V45L+A93S+L149R+A252S+H334D;

V45L+A93S+L149R+A252S+H334D+A340G;

V45L+L149R+S190E+D446T+R459V;

A25W+V45L+L149R+V258P+A276S+H358T;

A25W+V45L+S92D+L149R+S214E+V258P+A276S+A364G;

A25W+V45L+L149R+S214E+V258P+A276S+V350L+A364G;

A25W+V45L+L149R+S214Q+V258P+A276S+A364G;

V45L+L149R+N168G+V258P+A276S+A364G+S388K;

V45L+A117S+L149R+V258P+A276S+A364G+S388K;

A25W+V45L+L149R+V258P+A276S+H358T+A364G;

V45L+L149R+V258P+N260H+A276S+A364G+S388K;

V45L+L149R+T215I+V258P+A276S+A364G+S388K;

A25W+V45L+L149R+V258P+A276Q+A364G;

V45L+T70R+A117S+L149R+C161L+S214Q+V258P+A276S+L281V+H358D+A364G+S388K+G394A+D470T+R483V;

A25W+V45L+L149R+S214E+V258P+A276S;

V45L+A117S+L149R+S214Q+V258P+A276S+A286S+H358D+A364G+S388K;

V45L+L149R+T211H+S214Q+V258P+A276S+A364G+S388K;

V45L+Q61K+L149R+V258P+A276S+H358T+A364G;

V45L+L149R+V258P+A276S+Q314A+H358T+A364G;

V45L+L149R+T180Q+V258P+A276S+A364G+S388K;

V45L+L149R+V258P+A276S+Q314A+A364G+S388K;

A25W+V45L+L149R+V258P+A276S+A286S+A364G;

V45L+L149R+S190E+D446T;

V45L+L149R+V258P+A276S+A364G;

V45L+L149R+A170S+V258P+A276S+A364G;

V45L+L149R+V258P+A276Q+A364G;

V45L+T70R+A117S+L149R+S214Q+V258P+A276S+L281V+H358D+A364G+S388K+G394A+D470T+R483V+C522A;

V45L+L149R+H219I+V258P+A276S+H358T+A364G;

V45L+L149R+V258P+A276S+V350L+A364G+S388K;

D11E+V45L+L149R+V258P+A276S+A364G+S388K;

V45L+D113T+L149R+V258P+A276S+A364G+S388K;

V45L+L149R+G187K+V258P+A276S+A364G+S388K;

M28P+V45L+L149R+V258P+A276S+A364G+S388K;

V45L+Q61R+A117S+L149R+S214Q+V258P+A276S+H358D+A364G+S388K+D470T+R483V;

A25W+V45L+L149R+S214Q+V258P+A276S;

V45L+A117S+L149R+S214Q+T215I+V258P+A276S+A364G+S388K;

A25W+V45L+L149R+V258P+A276S+S388K;

A25W+V45L+L149R+S214E+H219I+V258P+A276S+A364G;

V45L+A93S+L149R+V234P+A252S+H334D;

V45L+L149R+T211H+V258P+A276S+H358T+A364G+S388K;

A25W+V45L+A117S+L149R+S214E+V258P+A276S+A364G;

V45L+L149R+S173Y+V258P+A276S+A364G+S388K;

A25W+V45L+L149R+V258P+A276S+Q314A+A364G;

V45L+L149R+G187L+V258P+A276S+H358T+A364G;

V45L+L149R+T215V+V258P+A276S+H358T+A364G;

V45L+T70R+L149R+V258P+A276S+A364G+S388K;

V45L+N127D+L149R+V258P+A276S+A364G;

V45L+A117S+L149R+S214Q+V258P+A276S+A364G+S388K+Q496R;

V45L+L149R+W250Y+V258P+A276S+A364G;

V45L+L149R+V258P+A276S+V350L+H358T+A364G;

D9E+V45L+L149R+V258P+A276S+H358T+A364G;

V45L+L149R+T211H+V258P+A276S+H358T+A364G;

A25W+V45L+L149R+W250Y+A276Q+A364G;

V45L+L149R+V258P+A276S+L281V+A364G+S388K;

V45L+L149R+V258P+A276S+S282N+H358T+A364G;

V45L+L149R+T215I+V258P+A276S+H358T+A364G;

A25W+V45L+L149R+S173Y+V258P+A276S+A364G;

V45L+Q61K+L149R+V258P+A276S+A364G+S388K;

V45L+L149R+T180Q+V258P+A276S+A364G;

V45L+A117S+L149R+A170S+S214Q+V258P+A276S+A364G+S388K;

V45L+Q61R+A117S+L149R+S214Q+V258P+A276S+A364G+S388K;

A25W+V45L+L149R+S214E+T215V+V258P+A276S+A364G;

V45L+A117S+L149R+S214Q+V258P+A276S+A364G+S388K+V497P;

V45L+L149R+V258P+A276S+Q314A+A364G;

V45L+A117S+L149R+V258P+A276S+A364G;

V45L+A117S+L149R+V258P+A276S+H358T+A364G+S388K;

V45L+L149R+N168G+V258P+A276S+H358T+A364G+S388K;

A25W+V45L+Q61K+L149R+V258P+A276S+A364G;

V45L+L149R+S214E+V258P+A276S+L281V+A364G;

V45N+L149R+V258P+A276S+A364G+S388K;

V45L+L149R+V258P+A276S+A286E+H358T+A364G;

D9E+V45L+L149R+V258P+A276S+A364G+S388K;

V45L+S92D+L149R+V258P+A276S+A364G+S388K;

V45L+A117S+L149R+T180Q+S214Q+V258P+A276S+A364G+S388K;

V45L+A117S+L149R+S214Q+V258P+A276S+A364G+S388H;

V45L+A117S+L149R+S214Q+T215V+V258P+A276S+A364G+S388K;

V45L+A117S+L149R+S214Q+V258P+A276S+V350L+A364G+S388K;

A25W+V45L+L149R+S214E+T215I+V258P+A276S+A364G;

V45L+N127D+L149R+V258P+A276S+H358T+A364G+S388K;

A25W+V45L+L149R+V258P+A276S+A286E+A364G;

V45L+L149R+N168G+V258P+A276S+L281V+A364G;

V45L+Q61R+L149R+V258P+A276S+H358T+A364G;

V45L+L149R+G187S+V258P+A276S+A364G+S388K;

V45L+L149R+V258P+A276S+L281V+Q314A+A364G;

V45L+L149R+V258P+A276S+L281V+H358T+A364G;

V45L+L149R+A170S+V258P+A276S+H358T+A364G;

V45L+L149R+A170S+V258P+A276S+A364G+S388K;

V45L+L149R+T215V+V258P+A276S+A364G+S388K;

V45L+L149R+V258P+A276Q+L281V+A364G;

V45L+T70R+A117S+L149R+S214Q+C243S+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V+C522V;

A25W+V45L+L149R+V258P+A276S+Q314A;

V45L+L149R+V258P+A276Q+H358T+A364G+S388K;

A25W+V45N+L149R+S214E+V258P+A276S+A364G;

V45L+L149R+V258P+A276S+A286E+A364G;

V45L+L149R+V258P+A276S+A286S+H358T+A364G;

A25W+V45L+L149R+G187S+V258P+A276S+A364G;

V45L+L149R+T180Q+V258P+A276S+L281V+A364G;

A25W+V45L+L149R+A276Q+L281V+A364G;

V45L+S92D+L149R+V258P+A276S+L281V+A364G;

V45L+Q61R+L149R+V258P+A276S+L281V+A364G;

V45L+L149R+A170S+V258P+A276S+L281V+A364G;

D11E+A25W+V45L+L149R+V258P+A276S+A364G;

A25W+V45L+Q61R+L149R+V258P+A276S+A364G;

A25W+V45L+L149R+T215V+V258P+A276S+A364G;

D9E+V45L+L149R+V258P+A276S+L281V+A364G;

A25W+V45L+L149R+V258P+A276S+V350L+A364G;

V45L+Q61K+A117S+L149R+S214Q+V258P+A276S+A364G+S388K;

V45L+A117S+L149R+S214Q+V258P+A276S+A364G+S388K+Y391V;

V45L+A117S+L149R+N168G+S214Q+V258P+A276S+A364G+S388K;

V45L+D113T+A117S+L149R+S214Q+V258P+A276S+A364G+S388K;

A25W+V45L+L149R+V258P+A276S+A286E;

A25W+V45L+L149R+V258P+A276S+L281V;

A25W+V45L+D113T+L149R+V258P+A276S;

V45L+L149R+H219V+V258P+A276S+L281V+A364G;

V45L+L149R+T180Q+V258P+A276S+H358T+A364G;

V45L+L149R+T215V+V258P+A276S+L281V+A364G;

A25W+V45L+L149R+V258P+A276S+S282N+A364G;

A25W+V45L+L149R+T215I+V258P+A276S+A364G;

A25W+V45L+L149R+H219I+V258P+A276S+A364G;

V45L+A117S+L149R+V258P+A276S+L281V+A364G;

D9E+A25W+V45L+L149R+V258P+A276S+A364G;

V45L+L149R+S214Y+V258P+A276S+A364G+S388K;

A25W+V45L+L149R+T180Q+V258P+A276S+A364G;

A25W+V45L+L149R+V258P+A276S+L281V+A364G;

V45L+N127D+L149R+V258P+A276S+L281V+A364G;

V45L+L149R+S173Y+V258P+A276S+H358T+A364G;

V45L+Q61R+L149R+V258P+A276S+A364G+S388K;

M28P+V45L+A117S+L149R+S214Q+V258P+A276S+A364G+S388K;

V45L+A117S+L149R+S214Q+V258P+A276S+A364G+S388A;

V45N+A117S+L149R+S214Q+V258P+A276S+A364G+S388K;

V45L+A117S+L149R+S214Q+V258P+A276S+A364G+S388K+T477K;

A25W+V45L+L149R+T180Q+V258P+A276S;

A25W+V45L+L149R+V258P+A276S+A286S;

A25W+V45L+L149R+G187S+S214E+V258P+A276S+A364G;

V45L+L149R+V258P+A276S+L281V+A286E+A364G;

A25W+V45L+L149R+W250Y+V258P+A276S+A364G;

A25W+V45L+L149R+V258P+N260H+A276S+A364G;

V45L+L149R+V258P+A276S+L281V+V350L+A364G;

A25W+V45L+L149R+A170S+V258P+A276S+A364G;

V45L+L149R+V258P+A276S+L281V+A286S+A364G;

V45L+L149R+V258P+A276S+L281V+S282N+A364G;

D11E+V45L+L149R+V258P+A276S+L281V+A364G;

V45L+Q61K+L149R+V258P+A276S+L281V+A364G;

V45L+L149R+T215I+V258P+A276S+L281V+A364G;

A25W+V45L+S92D+L149R+V258P+A276S+A364G;

V45L+L149R+S214Y+V258P+A276S+L281V+A364G;

V45L+L149R+T211H+V258P+A276S+L281V+A364G;

A25W+V45L+L149R+G187L+V258P+A276S+A364G;

V45L+S92D+L149R+V258P+A276S+H358T+A364G;

V45L+L149R+G187L+A276Q+L281V+A364G;

V45L+L149R+A276Q+L281V+V350L+A364G;

V45L+L149R+A276Q+L281V+A364G+S388K;

V45L+A117S+L149R+S214Y+V258P+A276S+A364G+S388K;

A25W+V45L+S92D+L149R+V258P+A276S;

A25W+V45L+L149R+G187L+V258P+A276S;

A25W+V45L+L149R+W250Y+V258P+A276S;

V45L+D113T+L149R+V258P+A276S+A364G;

V45L+L149R+G187K+V258P+A276S+H358T+A364G;

V45L+L149R+N163H+V258P+A276S+L281V+A364G;

A25W+V45L+T70R+L149R+V258P+A276S+A364G;

V45L+L149R+S173Y+V258P+A276S+L281V+A364G;

V45L+L149R+G187L+V258P+A276S+L281V+A364G;

V45L+L149R+G187K+V258P+A276S+L281V+A364G;

V45L+L149R+W250Y+V258P+A276S+H358T+A364G;

V45L+L149R+T211H+V258P+A276S+A364G+S388K;

V45L+L149R+N163H+V258P+A276S+A364G+S388K;

V45L+T70R+L149R+A276Q+L281V+A364G;

V45L+S92D+L149R+A276Q+L281V+A364G;

V45L+D113T+L149R+A276Q+L281V+A364G;

V45L+L149R+A276Q+L281V+Q314A+A364G;

V45L+A117S+L149R+S214Q+V258P+A276S+A364G+S388K+S389V;

A25W+V45L+Q61K+L149R+V258P+A276S;

D9E+A25W+V45L+L149R+V258P+A276S;

V45L+T70R+L149R+V258P+A276S+A364G;

V45L+L149R+V258P+A276S+A286S+A364G+S388K;

A25W+V45L+L149R+S214Y+V258P+A276S+A364G;

A25W+V45L+D113T+L149R+V258P+A276S+A364G;

V45L+T70R+L149R+V258P+A276S+L281V+A364G;

A25W+V45L+N127D+L149R+V258P+A276S+A364G;

V45L+T70R+L149R+V258P+A276S+H358T+A364G;

V45L+L149R+N163H+V258P+A276S+A364G;

V45L+L149R+T180Q+A276Q+L281V+A364G;

V45L+L149R+A276Q+L281V+A286S+A364G;

A25W+V45L+L149R+A170S+V258P+A276S;

A25W+V45L+T70R+L149R+V258P+A276S;

A25W+V45L+L149R+S214E+V258P+A276S+A286S+A364G;

V45L+L149R+V258P+A276S+A286S+H358T+A364G+S388K;

V45L+L149R+H219I+V258P+A276S+L281V+A364G;

A25W+V45L+A117S+L149R+V258P+A276S+A364G;

A25W+V45L+L149R+N168G+V258P+A276S+A364G;

V45L+L149R+N168G+A276Q+L281V+A364G;

V45L+L149R+S173Y+A276Q+L281V+A364G;

V45L+L149R+W250Y+A276Q+L281V+A364G;

V45L+A117S+L149R+S173Y+S214Q+V258P+A276S+A364G+S388K;

A25W+V45L+L149R+A276Q+A286E+A364G;

A25W+V45L+L149R+A276Q+A364G+S388K;

V45L+N127D+L149R+V258P+A276S+H358T+A364G;

A25W+V45L+L149R+V258P+A276S;

V45L+N127D+L149R+A276Q+L281V+A364G;

V45L+L149R+A276Q+L281V+A286E+A364G;

V45L+A117S+L149R+S214Q+V258P+A276S+A364G+S388K+V500T;

A25W+V45L+A117S+L149R+V258P+A276S;

A25W+V45L+L149R+H219V+V258P+A276S+A364G;

V45L+L149R+S173Y+V258P+A276S+H358T+A364G+S388K;

D11E+V45L+L149R+V258P+A276S+H358T+A364G;

V45L+A79C+L149R+V258P+A276S+N355G+A364G+S388K;

A24K+V45L+L149R+V258P+A276S+A364G;

V45L+L149R+V258P+A276S+S282N+A364G;

V45L+A117S+L149R+A276Q+L281V+A364G;

V45L+L149R+N260H+A276Q+L281V+A364G;

V45L+A117S+L149R+T211H+S214Q+V258P+A276S+A364G+S388K;

A25W+V45L+L149R+N168G+V258P+A276S;

A25W+V45L+L149R+G187S+V258P+A276S;

A25W+V45L+L149R+V258P+A276S+V350L;

A25W+V45L+Q61R+L149R+V258P+A276S;

A25W+V45L+L149R+G187L+S214E+V258P+A276S+A364G;

V45L+L149R+G187S+V258P+A276S+L281V+A364G;

A25W+V45L+L149R+N163H+V258P+A276S+A364G;

A25W+V45L+L149R+G187K+V258P+A276S+A364G;

V45N+L149R+V258P+A276S+L281V+A364G;

V45L+L149R+A276Q+L281V+A364G;

V45L+L149R+V258P+N260H+A276S+H358T+A364G;

V45L+L149R+A276Q+S282N+A364G;

V45L+S92D+A117S+L149R+S214Q+V258P+A276S+A364G+S388K;

A25W+V45L+L149R+V258P+A276S+S282N;

A25W+V45L+L149R+S173Y+V258P+A276S;

A25W+V45L+L149R+A276Q+A286S+A364G;

V45L+D113T+L149R+V258P+A276S+L281V+A364G;

V45L+L149R+S214Q+V258P+A276S+L281V+A364G+S388K;

V45L+D113T+L149R+V258P+A276S+H358T+A364G;

A25W+V45L+L149R+T215V+V258P+A276S;

V45L+L149R+N168G+V258P+A276S+H358T+A364G;

V45L+A117S+L149R+S214Q+V258P+A276S+A364G+S388K+A472L;

V45L+A117S+L149R+S214Q+V258P+A276S+A364G+S388K+S473K;

A25W+V45L+L149R+T215I+V258P+A276S;

A25W+V45L+L149R+T211H+V258P+A276S;

A25W+V45L+L149R+V258P+A276Q;

V45L+L149R+S214Q+T215V+V258P+A276S+A364G+S388K;

V45L+L149R+N163H+V258P+A276S+H358T+A364G+S388K;

V45L+A117S+L149R+S214Q+V258P+A276S+A364G+S388K+A499L;

V45L+L149R+V258P+A276S+V350L+A364G;

V45L+A79C+A117S+L149R+V258P+A276S+A364G+S388K;

A25W+V45L+D113T+L149R+A276Q+A364G;

A25W+V45N+L149R+V258P+A276S+A364G;

V45L+T70R+L149R+A276Q+A364G;

A25W+V45L+L149R+H219I+V258P+A276S;

V45L+A117S+L149R+S214Q+V258P+A276S+A364G+S388K+V498I;

M28P+V45L+L149R+V258P+A276S+A364G;

A25W+V45L+T70R+L149R+A276Q+A364G;

A25W+V45L+L149R+A276Q+Q314A+A364G;

V45L+L149R+A170S+V258P+A276S+H358T+A364G+S388K;

A25W+V45L+L149R+S214Y+V258P+A276S;

V45L+L149R+S214Y+V258P+A276S+H358T+A364G;

V45L+L149R+A276Q+L281V+H358T+A364G;

A25W+V45L+L149R+H219V+V258P+A276S;

V45L+A117S+L149R+N163H+S214Q+V258P+A276S+A364G+S388K;

D11E+A25W+V45L+L149R+V258P+A276S;

V45L+A117S+L149R+S214Q+V258P+A276S+A364G+S388K+N527H;

A25W+V45L+A117S+L149R+A276Q+A364G;

V45L+L149R+V258P+L281V+A364G;

A25W+V45L+L149R+G187L+A276Q+A364G;

V45N+L149R+V258P+A276S+H358T+A364G;

V45L+A117S+L149R+S214Q+V258P+A276S+N355G+H358D+A364G+S388K+D470T+R483V;

V45L+A117S+L149R+S214Q+V258P+A276S+H358D+A364G+S388H+D470T+R483V;

A25W+V45L+L149R+A276Q+A364G;

V45L+A117S+L149R+S214Q+V258P+A276S+H358D+A364G+S388K+D470T+R483V+V497E;

A25W+V45L+L149R+N163H+A276Q+A364G;

A25W+V45L+L149R+S173Y+A276Q+A364G;

A25W+V45L+L149R+A276Q+S282N+A364G;

A25W+V45L+L149R+A276Q+H358T+A364G;

A25W+V45L+L149R+T180Q+A276Q+A364G;

V45L+A117S+L149R+V258P+A276S+H358T+A364G;

V45L+L149R+A276Q+Q314A+A364G;

V45L+L149R+G187K+V258P+A364G;

V45L+L149R+A276Q+A286E+A364G;

A25W+V45L+N127D+L149R+A276Q+A364G;

A25W+V45L+L149R+A170S+A276Q+A364G;

A25W+V45L+N127D+L149R+V258P+A276S;

V45L+A117S+L149R+S214Q+V258P+A276S+A364G+S388K+T477V;

V45L+A117S+L149R+S214Q+V258P+A276S+A364G+S388K+G503L;

A25W+M28P+V45L+L149R+V258P+A276S+A364G;

V45L+L149R+A276Q+H358T+A364G;

A25W+V45L+L149R+N168G+A276Q+A364G;

A25W+V45L+L149R+N260H+A276Q+A364G;

V45L+A117S+L149R+G187L+S214Q+V258P+A276S+A364G+S388K;

V45L+A117S+L149R+N163H+S214Q+V258P+A276S+H358D+A364G+S388K+D470T+L530R;

V45L+A117S+L149R+S214Q+T215K+V258P+A276S+A364G+S388K;

A25W+V45L+L149R+G187K+V258P+A276S;

A25W+M28P+V45L+L149R+V258P+A276S;

A25W+V45N+L149R+V258P+A276S;

V45L+A117S+L149R+S214Q+V258P+A276S+N355G+H358D+A364G+S388K+D470T;

V45L+N127D+L149R+A276Q+A364G;

V45L+L149R+W250Y+A276Q+A364G;

V45L+L149R+W250Y+V258P+A364G;

V45L+L149R+N163H+A276Q+L281V+A364G;

V45L+A117S+L149R+S214Q+V258P+A276S+A364G+Q385C+S388K;

A25W+V45L+L149R+S214E+T215K+V258P+A276S+A364G;

M28P+V45L+L149R+V258P+A276S+L281V+A364G;

V45L+L149R+A276Q+A364G+S388K;

A25W+V45L+L149R+V258P+A364G;

M28P+V45L+L149R+V258P+A276S+H358T+A364G;

V45L+A117S+L149R+S214Q+V258P+A276S+N355G+H358D+A364G+S388K;

V45L+T70R+A117S+L149R+S214Q+V258P+A276S+L281V+H358D+A364G+S388K+G394A+D470T+R483V+C540V;

V45L+L149R+N168G+A276Q+A364G;

V45L+A117S+L149R+A276Q+A364G;

V45L+L149R+N163H+V258P+A276S+H358T+A364G;

V45L+L149R+T215K+V258P+A276S+H358T+A364G;

A24K+V45L+L149R+A276Q+A364G;

V45L+L149R+A276Q+A286S+A364G;

V45L+L149R+V258P+A364G;

V45L+L149R+H219I+V258P+A276S+A364G+S388K;

V45L+L149R+A170S+A276Q+A364G;

V45L+L149R+T180Q+A276Q+A364G;

V45L+L149R+A276Q+V350L+A364G;

V45L+L149R+V258P+S282N+A364G;

V45L+L149R+A276Q+A364G;

V45L+L149R+A276S+A364G;

V45L+L149R+T215K+V258P+A276S+A364G+S388K;

V45L+A117S+L149R+S214Q+V258P+A276S+N355G+A364G+S388K+D470T;

V45L+L149R+V258P+A276S;

A25W+V45L+L149R+A276S+A364G;

V45L+L149R+G187S+V258P+A364G;

V45L+S92D+L149R+V258P+A276S+H358T+A364G+S388K;

A25W+L149R+V258P+A276S+A364G;

V45L+L149R+G187L+A276Q+A364G;

A25W+V45L+V258P+A276S+A364G;

V45L+L149R+N260H+A276Q+A364G;

V45L+L149R+W250Y+V258R+A364G;

V45L+L149R+N163H+A276Q+A364G;

V45L+S92D+L149R+A276Q+A364G;

V45L+L149R+S173Y+A276Q+A364G;

V45L+S92D+L149R+V258P+A364G;

V45L+L149R+W250Y+V258P+A276S+L281V+A364G V45L+L149R+L281V+A364G; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by TSA of at least 5° C. compared to the xylanase of SEQ ID NO: 1.

In one aspect, the present invention relates to xylanase variants comprises at least one of the following substitutions or combinations of substitutions:

V45L+Q61K+T70R+A117S+L149R+S214Q+V258P+A276S+L281V+A286E+H358D+A364G+Q384A+S388K+V393H+D470T+R483V;

V45L+Q61K+T70R+A117S+L149R+S214Q+V258P+A276S+L281V+H358D+A364G+Q384A+S388K+G394Q+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+A286E+H358D+A364G+Q384A+S388K+V393H+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+A79C+A117S+L149R+S214Q+V258P+A276S+H358D+A364G+S388K+D470T+L530R;

V45L+A79C+A117S+L149R+S214Q+V258P+A276S+H358D+A364G+S388K+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+Q61K+A117S+L149R+S214Q+V258P+A276S+L281V+H358D+A364G+S388K+G394A+D470T+R483V+L530R;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+Q61K+A117S+L149R+S214Q+V258P+A276S+L281V+H358D+A364G+S388K+G394A+D470T+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+D470T+R483V+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+D470T+R483V+L530R;

V45L+A117S+L149R+G187K+S214Q+V258P+A276S+A286E+H358D+A364G+Q384A+S388K+V393H+D470T+R483V;

V45L+A117S+L149R+G187K+S214Q+V258P+A276S+A286E+H358D+A364G+Q384A+S388K+N395D+D470T+R483V;

V45L+A117S+L149R+G187K+S214E+T215I+V258P+A276S+A286E+H358D+A364G+S388K+V393H+D470T+R483V;

V45L+A117S+L149R+G187K+S214E+T215I+V258P+A276S+A286E+H358D+A364G+S388K+N395D+D470T+R483V;

V45L+A117S+L149R+S214Q+V258P+A276S+H358D+A364G+Q385C+S388K+D470T+R483V;

V45L+Q61K+A117S+L149R+S214Q+V258P+A276S+H358D+A364G+S388K+D470T+R483V;

V45L+A79C+A117S+L149R+S214Q+V258P+A276S+A364G+S388K+D470T+R483V;

V45L+A117S+L149R+S214Q+V258P+A276S+L281V+H358D+A364G+S388K+D470T+R483V;

V45L+A117S+L149R+S214Q+V258P+A276S+H358D+A364G+T366I+S388K+D470T+R483V;

V45L+T70R+A117S+L149R+S214Q+V258P+A276S+L281V+H358D+A364G+S388K+G394A+D470T+R483V;

V45L+A117S+L149R+S214Q+V258P+A276S+H358D+A364G+Q384A+S388K+D470T+L530R;

V45L+Q61K+A117S+L149R+S214Q+V258P+A276S+H358D+A364G+S388K+D470T+L530R;

V45L+T70R+A117S+L149R+S214Q+V258P+A276S+H358D+A364G+S388K+D470T+R483V;

V45L+A117S+L149R+S214Q+V258P+A276S+H358D+A364G+S388K+V393H+D470T+R483V;

V45L+A117S+L149R+S214Q+V258P+A276S+H358D+A364G+S388K+G394A+D470T+R483V;

V45L+A117S+L149R+S214Q+V258P+A276S+H358D+A364G+S388K+V393H+D470T+L530R;

V45L+A117S+L149R+S214Q+V258P+A276S+A286E+H358D+A364G+S388K+D470T+R483V;

D11E+V45L+A117S+L149R+S214Q+V258P+A276S+H358D+A364G+S388K+D470T+R483V;

V45L+T70R+A117S+L149R+S214Q+V258P+A276S+L281V+H358D+A364G+S388K+G394A+C468V+D470T+R483V;

V45L+A79C+A117S+L149R+S214Q+V258P+A276S+H358D+A364G+S388K+D470T V45L+A79C+L149R+S214E+V258P+A276S+A364G+S388K;

V45L+A117S+L149R+G187L+S214Q+V258P+A276S+H358D+A364G+S388K+D470T+L530R;

V45L+A117S+L149R+S214Q+V258P+A276S+H358D+A364G+Q384A+S388K+D470T+R483V;

V45L+A117S+L149R+G187K+S214Q+V258P+A276S+H358D+A364G+S388K+D470T+R483V;

V45L+A117S+L149R+S214Q+V258P+A276S+L281V+H358D+A364G+S388K+D470T+L530R;

V45L+A117S+L149R+S214E+V258P+A276S+H358D+A364G+S388K+D470T+R483V;

V45L+A117S+L149R+G187L+S214Q+V258P+A276S+H358D+A364G+S388K+D470T+R483V;

V45L+A117S+L149R+S214Q+V258P+A276S+H358D+A364G+S388K+G394Q+D470T+R483V;

V45L+A117S+L149R+S214Q+V258P+A276S+H358D+A364G+S388K+G394A+D470T+L530R;

V45L+A117S+L149R+S214Q+V258P+A276S+H358D+A364G+T366V+S388K+D470T+L530R;

V45L+A117S+L149R+S214Q+V258P+A276S+H358D+A364G+S388K+G394Q+D470T+L530R;

V45L+A117S+L149R+S214Q+V258P+A276S+S282N+H358D+A364G+S388K+D470T+R483V;

V45L+A117S+L149R+S214Q+V258P+A276S+H358D+A364G+S388K+N395D+D470T+R483V;

V45L+A117S+L149R+S214Q+V258P+A276S+H358D+A364G+S388K+D470T+N476D+R483V;

V45L+A117S+L149R+S214Q+V258P+A276S+V350L+H358D+A364G+S388K+D470T+R483V;

V45L+A117S+L149R+S214Q+V258P+A276S+H358D+A364G+S388K+D470T+T477K+R483V;

V45L+A117S+L149R+S214Q+V258P+A276S+H358D+A364G+S388K+D470T+R483V+Q496R;

V45L+T70R+A117S+L149R+S214Q+V258P+A276S+L281V+H358D+A364G+S388K+G394A+C468A+D470T+R483V;

V45L+A117S+L149R+S214Q+V258P+A276S+H358D+A364G+S388K+D470T+R483V+W495E;

V45L+A117S+L149R+S214Q+V258P+A276S+A286E+H358D+A364G+S388K+D470T+L530R;

V45L+A117S+L149R+G187K+S214Q+V258P+A276S+H358D+A364G+S388K+D470T+L530R;

V45L+A117S+L149R+S214Q+V258P+A276S+H358D+A364G+S388K+Y391V+D470T+R483V;

V45L+A117S+L149R+S214Q+V258P+A276S+H358D+A364G+S388K+D470T+R483V;

D9E+V45L+A117S+L149R+S214Q+V258P+A276S+H358D+A364G+S388K+D470T+L530R;

V45L+A117S+L149R+S214Q+V258P+A276S+H358D+A364G+S388K+D470T+T477V+R483V;

V45L+A117S+L149R+S214Q+V258P+A276S+H358D+A364G+S388K+D470T+N476N+R483V;

V45L+A117S+L149R+S214Q+V258P+A276S+H358D+A364G+S388K+V393R+D470T+R483V;

V45L+A117S+L149R+S214Q+V258P+A276S+H358D+A364G+S388K+D470T+T480L+L530R;

V45L+A117S+L149R+S214Q+V258P+A276S+H358D+A364G+S388K+D470T+S481A+L530R;

V45L+A117S+L149R+S214Q+W250Y+V258P+A276S+H358D+A364G+S388K+D470T+R483V;

V45L+A117S+L149R+S214Q+V258P+A276S+H358D+A364G+S388K+D470T+S481S+R483V;

V45L+A117S+L149R+S173Y+S214Q+V258P+A276S+H358D+A364G+S388K+D470T+R483V;

V45L+T70R+A117S+L149R+S214Q+C243V+V258P+A276S+L281V+H358D+A364G+S388K+G394A+D470T+R483V;

V45L+A117S+L149R+T180Q+S214Q+V258P+A276S+H358D+A364G+S388K+D470T+L530R;

D11E+V45L+A117S+L149R+S214Q+V258P+A276S+H358D+A364G+S388K+D470T+L530R;

V45L+A117S+L149R+S214E+V258P+A276S+H358D+A364G+S388K+D470T+L530R;

V45L+A117S+L149R+S214Q+V258P+A276S+H358D+A364G+S388K+D470T+N476D+L530R;

V45L+A117S+L149R+S214Q+V258P+A276S+A286S+H358D+A364G+S388K+D470T+R483V;

V45L+A117S+L149R+S214Q+V258P+A276S+H358D+A364G+S388K+D470T+R483V+Q496Q;

V45L+A117S+L149R+S214Q+V258P+A276Q+H358D+A364G+S388K+D470T+R483V;

V45L+A117S+L149R+S214Q+V258P+A276S+H358D+A364G+S388K+D470T+R483V+Q496E;

V45L+Q61R+A117S+L149R+S214Q+V258P+A276S+H358D+A364G+S388K+D470T+L530R;

V45L+A117S+L149R+S214Q+V258P+A276S+Q314A+H358D+A364G+S388K+D470T+L530R;

V45L+A117S+L149R+S214Q+V258P+A276Q+H358D+A364G+S388K+D470T+L530R;

V45L+A117S+L149R+S214Q+V258P+A276S+H358D+A364G+S388K+D470T+L478P+R483V;

A25W+V45L+A117S+L149R+S214Q+V258P+A276S+H358D+A364G+S388K+D470T+R483V;

V45L+A117S+L149R+T180Q+S214Q+V258P+A276S+H358D+A364G+S388K+D470T+R483V;

V45L+A117S+L149R+S214Q+V258P+A276S+H358D+A364G+S388K+D470T+R483V+L484K;

V45L+A117S+L149R+S214Q+V258P+A276S+H358D+A364G+S388K+D470T+N474G+L530R;

V45L+A117S+L149R+S214Q+V258P+A276S+H358D+A364G+S388K+N395D+D470T+L530R;

V45L+A117S+L149R+S214Q+V258P+A276S+H358D+A364G+T366V+S388K+D470T+R483V;

V45L+A117S+L149R+N168G+S214Q+V258P+A276S+H358D+A364G+S388K+D470T+R483V;

V45L+A117S+L149R+S214Q+V258P+A276S+H358D+A364G+S388K+D470T+R483V+L484S;

V45L+A117S+L149R+S214Q+V258P+A276S+H358D+A364G+S388K+D470T+L530R;

V45L+A117S+L149R+S214Q+V258P+A276S+H358D+A364G+S388K+D470T+R483V+L484D;

V45L+A117S+L149R+S214Q+V258P+A276S+H358D+A364G+S388K+D470T+R483V+S528H;

V45L+A117S+L149R+S214Q+V258P+A276S+H358D+A364G+S388K+D470T+R483V+S501N;

V45L+A117S+L149R+S173Y+S214Q+V258P+A276S+H358D+A364G+S388K+D470T+L530R;

V45L+A117S+L149R+S214Q+V258P+A276S+H358D+A364G+S388K+D470T+R483V+A499L;

V45L+S92D+A117S+L149R+S214Q+V258P+A276S+H358D+A364G+S388K+D470T+R483V;

V45L+A117S+L149R+S214Q+V258P+A276S+Q314A+H358D+A364G+S388K+D470T+R483V;

V45L+A79C+L149R+S214Q+V258P+A276S+A364G+S388K;

V45L+A117S+L149R+S214Q+V258P+A276S+N355G+H358D+A364G+S388K+D470T+L530R;

V45L+A117S+L149R+S214Q+V258P+A276S+V350L+H358D+A364G+S388K+D470T+L530R;

V45L+A117S+L149R+S214Q+V258P+A276S+H358D+A364G+S388K+D470T+S481S+L530R;

V45L+A117S+L149R+S214Q+V258P+A276S+H358D+A364G+S388K+D470T+Q496E+L530R;

V45L+A117S+L149R+N168G+S214Q+V258P+A276S+H358D+A364G+S388K+D470T+L530R;

V45L+A117S+L149R+S214Q+V258P+A276S+H358D+A364G+S388K+D470T+V498I+L530R;

V45L+A117S+L149R+S214Q+V258P+A276S+H358D+A364G+S388K+D470A+R483V;

V45N+A117S+L149R+S214Q+V258P+A276S+H358D+A364G+S388K+D470T+R483V;

V45L+A117S+L149R+S214Q+V258P+A276S+H358D+A364G+S388K+D470T+R483V+A499Y;

V45L+A79C+L149R+V258P+A276S+Q314A+A364G+S388K;

V45L+A79C+L149R+V258P+A276S+L281V+A364G+S388K;

V45L+A117S+L149R+S214Q+V258P+A276S+A286S+H358D+A364G+S388K+D470T+L530R;

V45L+A117S+L149R+S214Q+V258P+A276S+H358D+A364G+S388K+D470T+S501N+L530R;

V45L+A117S+L149R+S214Q+V258P+A276S+H358D+A364G+S388K+D470T+N476N+L530R;

V45L+A117S+L149R+S214Q+V258P+A276S+H358D+A364G+S388K+D470T+T477V+L530R;

V45L+A117S+L149R+S214Q+V258P+A276S+H358D+A364G+S388H+D470T+L530R;

V45L+A117S+L149R+S214Q+V258P+A276S+H358D+A364G+S388K+D470T+L478N+L530R;

V45L+S92D+A117S+L149R+S214Q+V258P+A276S+H358D+A364G+S388K+D470T+L530R;

V45L+A117S+L149R+S214Q+V258P+A276S+H358D+A364G+S388K+D470T+V500V+L530R;

V45L+A117S+L149R+S214Q+V258P+A276S+H358D+A364G+S388K+D470T+R483V+V500V;

V45L+A117S+L149R+S214Q+V258P+A276S+H358D+A364G+S388K+D470T+L478P+L530R;

V45L+A117S+L149R+S214Q+V258P+A276S+H358D+A364G+S388K+D470T+R483V+S501E;

V45L+A117S+L149R+S214Q+V258P+A276S+H358D+A364G+S388K+D470T+D525L+L530R;

V45L+A117S+L149R+S214Q+V258P+A276S+H358D+A364G+S388K+D470T+G526T+L530R;

V45L+A117S+L149R+S214Q+V258P+A276S+H358D+A364G+S388K+D470T+N527H+L530R;

V45L+A117S+L149R+S214Q+V258P+A276S+H358D+A364G+S388K+D470T+T477K+L530R;

M28P+V45L+A117S+L149R+S214Q+V258P+A276S+H358D+A364G+S388K+D470T+L530R;

D9E+V45L+A117S+L149R+S214Q+V258P+A276S+H358D+A364G+S388K+D470T+R483V;

V45L+A117S+L149R+S214Q+V258P+A276S+H358D+A364G+S388K+D470T+S501E+L530R;

V45L+A117S+L149R+S214Q+W250Y+V258P+A276S+H358D+A364G+S388K+D470T+L530R;

V45L+A117S+L149R+S214Q+V258P+A276S+H358D+A364G+S388K+D470T+A499L+L530R;

V45L+A117S+L149R+S214Q+V258P+A276S+H358D+A364G+S388K+D470T+A472I+L530R;

V45L+A117S+N127D+L149R+S214Q+V258P+A276S+H358D+A364G+S388K+D470T+R483V;

V45L+A117S+L149R+S214Q+V258P+A276S+H358D+A364G+S388K+D470T+V500T+L530R;

V45L+A117S+L149R+S214Q+V258P+A276S+H358D+A364G+S388K+D470T+R483V+N527H;

V45N+A117S+L149R+S214Q+V258P+A276S+H358D+A364G+S388K+D470T+L530R;

V45L+A117S+L149R+S214Q+V258P+A276S+H358D+A364G+S388K+D470T+T480N+L530R;

V45L+A117S+L149R+S214Q+V258P+A276S+H358D+A364G+S388K+D470T+R483V+V497P;

V45L+A117S+L149R+S214Q+V258P+A276S+H358D+A364G+S388K+D470T+R483V+V498I;

V45L+A79C+L149R+S173Y+V258P+A276S+A364G+S388K;

V45L+A117S+L149R+S214Q+V258P+A276S+H358D+A364G+S388K+D470T+T480N+R483V;

V45L+A117S+L149R+S214Q+H219V+V258P+A276S+H358D+A364G+S388K+D470T+L530R;

V45L+A117S+L149R+S214Q+V258P+A276S+H358D+A364G+S388A+D470T+L530R;

M28P+V45L+A117S+L149R+S214Q+V258P+A276S+H358D+A364G+S388K+D470T+R483V;

V45L+A117S+L149R+A170S+S214Q+V258P+A276S+H358D+A364G+S388K+D470T+R483V;

V45L+A79C+L149R+G187K+V258P+A276S+A364G+S388K;

V45L+A117S+L149R+S214Q+V258P+A276S+H358D+A364G+S388K+S389H+D470T+L530R;

V45L+D113T+A117S+L149R+S214Q+V258P+A276S+H358D+A364G+S388K+D470T+L530R;

V45L+A117S+L149R+N163H+S214Q+V258P+A276S+H358D+A364G+S388K+D470T+R483V;

V45L+A117S+L149R+S214Q+V258P+A276S+H358D+A364G+S388A+D470T+R483V;

V45L+A117S+L149R+S214Q+V258P+A276S+H358D+A364G+S388L+D470T+R483V;

V45L+Q61R+A79C+L149R+V258P+A276S+A364G+S388K;

V45L+A79C+L149R+N168G+V258P+A276S+A364G+S388K;

V45L+A117S+L149R+T211H+S214Q+V258P+A276S+H358D+A364G+S388K+D470T+L530R;

V45L+A79C+L149R+T215V+V258P+A276S+A364G+S388K;

V45L+A117S+L149R+S214Q+V258P+A276S+H358D+A364G+S388K+D470T+R483V+V500T;

V45L+A79C+L149R+V258P+A276S+A286S+A364G+S388K;

V45L+A79C+L149R+H219V+V258P+A276S+A364G+S388K;

V45L+A79C+L149R+A170S+V258P+A276S+A364G+S388K;

V45L+D113T+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A79C+L149R+V258P+A276S+A364G+S388K;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+T215I+V258P+A276S+H358D+A364G+S388K+D470T+R483V;

V45L+A117S+L149R+S214Q+H219I+V258P+A276S+H358D+A364G+S388K+D470T+R483V;

V45L+A117S+L149R+S214Q+V258P+A276S+H358D+A364G+S388K+D470T+L478N;

V45L+A117S+L149R+S214Q+V258P+A276S+L281V+H358D+A364G+S388K+D470T;

V45L+A117S+L149R+S214E+V258P+A276S+H358D+A364G+S388K+D470T;

V45L+T70R+A117S+L149R+S214Q+V258P+A276S+L281V+H358D+A364G+S388K+G394A+C468L+D470T+R483V;

V45L+A117S+L149R+S214Q+V258P+A276S+H358D+A364G+Q385C+S388K+D470T+L530R;

V45L+A117S+L149R+S214Q+V258P+A276S+H358D+A364G+T366I+S388K+D470T;

V45L+A117S+L149R+S214Q+V258P+A276S+H358D+A364G+S388K+D470T+S473G;

V45L+A117S+L149R+S214Q+V258P+A276S+H358D+A364G+S388K+D470T+L478P;

V45L+A117S+L149R+S214Q+V258P+A276S+H358D+A364G+S388K+G394D+D470T;

V45L+A117S+L149R+S214Q+V258P+A276S+A364G+Q384A+S388K+D470T+R483V;

D11E+V45L+A79C+L149R+V258P+A276S+A364G+S388K;

V45L+A117S+L149R+S214Q+V258P+A276S+H358D+A364G+S388K+D470T+R483V+L530R;

V45L+A117S+L149R+S214Q+V258P+A276S+H358D+A364G+S388K+D470T+N529T;

V45L+A117S+L149R+S214Q+V258P+A276S+H358D+A364G+S388K+G394Q+D470T;

V45L+A117S+L149R+G187L+S214Q+V258P+A276S+H358D+A364G+S388K+D470T;

V45L+A79C+L149R+V258P+A276S+A286E+A364G+S388K;

V45L+A117S+L149R+S214Q+V258P+A276S+H358D+A364G+S388K+D470T+R483V+G503L;

V45L+A117S+L149R+S214Q+V258P+A276S+Q314A+H358D+A364G+S388K+D470T;

V45L+A117S+L149R+S214Q+V258P+A276S+V350L+H358D+A364G+S388K+D470T;

V45L+A117S+L149R+G187K+S214Q+V258P+A276S+H358D+A364G+S388K+D470T;

V45L+A117S+L149R+S214Q+W250Y+V258P+A276S+H358D+A364G+S388K+D470T;

V45L+A117S+L149R+S214Q+V258P+A276S+A286E+H358D+A364G+S388K+D470T;

V45L+A117S+L149R+S214Q+V258P+A276S+H358D+A364G+Q384A+S388K+D470T;

V45L+A117S+L149R+S214Q+V258P+A276S+H358D+A364G+S388R+D470T;

V45L+A117S+L149R+S214Q+V258P+A276S+H358D+A364G+S388K+D470T+S528H, V45L+A117S+L149R+S214Q+T215V+V258P+A276S+H358D+A364G+S388K+D470T+R483V;

V45L+A117S+L149R+S214Q+V258P+A276S+H358D+A364G+S388K+D470T+Q496R;

V45L+A117S+L149R+N163H+S214Q+V258P+A276S+H358D+A364G+S388K+D470T;

V45L+A117S+L149R+S214Q+V258P+A276S+S282N+H358D+A364G+S388K+D470T;

V45L+A117S+L149R+S214Q+V258P+A276Q+H358D+A364G+S388K+D470T;

D11E+V45L+A117S+L149R+S214Q+V258P+A276S+H358D+A364G+S388K+D470T;

V45L+A117S+L149R+S214Q+V258P+A276S+H358D+A364G+T366V+S388K+D470T;

V45L+A117S+L149R+S214Q+V258P+A276S+H358D+A364G+S388K+D470T+S473K;

V45L+A79C+L149R+T180Q+V258P+A276S+A364G+S388K;

V45L+A117S+L149R+S214Q+V258P+A276S+H358D+A364G+S388K+D470T+S481A+R483V;

V45L+A117S+L149R+S214Q+V258P+A276S+H358D+A364G+S388R+D470T+R483V;

V45L+A117S+L149R+S214Q+V258P+A276S+Q314A+A364G+S388K+D470T+R483V;

V45L+S92D+A117S+L149R+S214Q+V258P+A276S+H358D+A364G+S388K+D470T;

V45L+A117S+L149R+S214Q+V258P+N260H+A276S+H358D+A364G+S388K+D470T;

V45L+A117S+L149R+G187S+S214Q+V258P+A276S+H358D+A364G+S388K+D470T;

V45L+A117S+L149R+S214Q+V258P+A276S+H358D+A364G+S388K+D470T+T477K;

V45L+A117S+L149R+S214Q+V258P+A276S+H358D+A364G+S388K+D470T+N474G;

V45L+A117S+L149R+S214Q+V258P+A276S+H358D+A364G+S388K+N395D+S501N;

V45L+A117S+N127D+L149R+S214Q+V258P+A276S+H358D+A364G+S388K+D470T;

V45L+A117S+L149R+S214Q+V258P+A276S+H358D+A364G+S388K+V393R+D470T;

V45L+A117S+L149R+S173Y+S214Q+V258P+A276S+H358D+A364G+S388K+D470T;

V45L+A117S+L149R+S214Q+V258P+A276S+H358D+A364G+S388H+D470T;

V45L+A117S+L149R+S214Q+T215V+V258P+A276S+H358D+A364G+S388K+D470T+L530R;

V45L+A117S+L149R+G187S+S214Q+V258P+A276S+H358D+A364G+S388K+D470T+L530R;

V45L+A117S+L149R+T211H+S214Q+V258P+A276S+H358D+A364G+S388K+D470T+R483V;

V45L+A117S+L149R+S214Q+V258P+A276S+H358D+A364G+S388K+D470T+T480N;

V45L+A117S+L149R+N168G+S214Q+V258P+A276S+H358D+A364G+S388K+D470T;

V45L+A79C+L149R+H219I+V258P+A276S+A364G+S388K;

V45L+L149R+S214E+V258P+A276S+H358D+A364G+D470T;

V45L+Q61K+A117S+L149R+S214Q+V258P+A276S+A364G+S388K+D470T+R483V;

V45L+D113T+A117S+L149R+S214Q+V258P+A276S+H358D+A364G+S388K+D470T;

V45L+A117S+L149R+S214Q+V258P+A276S+A364G+T366V+S388K+D470T+R483V;

V45L+A117S+L149R+S214Q+V258P+A276S+H358T+A364G+S388K+D470T+R483V;

V45L+A117S+L149R+S214Q+V258P+A276S+A364G+S388K+V393R+D470T+R483V;

V45N+A79C+L149R+V258P+A276S+A364G+S388K;

V45L+A117S+L149R+S214Q+V258P+A276S+H358D+A364G+S388K+D470T+Q496Q;

V45L+A117S+L149R+S214Q+V258P+A276S+A364G+S388K+G394Q+D470T+R483V;

V45L+A79C+L149R+V258P+A276S+V350L+A364G+S388K;

V45L+L149R+S214E+V258P+A276S+H358D+A364G+D470T;

V45L+A117S+L149R+S214Q+V258P+A276S+A364G+T366I+S388K+D470T+R483V;

V45L+A117S+L149R+S214Q+V258P+A276S+H358D+A364G+S388K+D470T+R483V+G526T;

V45L+A117S+L149R+S214Q+V258P+A276S+H358D+A364G+S388K+D470T+L484S;

V45L+A79C+A117S+L149R+S214Q+V258P+A276S+H358D+A364G+S388K;

V45L+A117S+L149R+S214Q+V258P+A276S+H358D+A364G+S388K+D470T+A499L;

V45L+A117S+L149R+S214Q+V258P+A276S+H358T+A364G+S388K+D470T+L530R;

V45L+A79C+D113T+L149R+V258P+A276S+A364G+S388K;

V45L+A117S+L149R+S214Q+V258P+A276S+H358D+A364G+S388K+D470T+R483V+D525L;

V45L+A117S+L149R+S214Q+V258P+A276S+H358D+A364G+S388K+D470T+V498I;

V45L+A117S+L149R+S214Q+V258P+A276S+H358D+A364G+S388L+D470T;

V45L+T70R+A117S+L149R+S214Q+V258P+A276S+A364G+S388K+D470T+R483V;

V45L+A117S+L149R+S214Q+V258P+A276S+H358D+A364G+S388K+D470T+S528H+L530R;

V45L+A117S+L149R+S214Q+V258P+A276S+A364G+S388K+D470T+N476D+R483V;

D9E+V45L+A117S+L149R+S214Q+V258P+A276S+A364G+S388K+D470T+R483V;

V45L+A117S+L149R+S214Q+V258P+A276S+H358D+A364G+S388K+D470T+S501E;

V45L+A117S+L149R+S214Q+V258P+A276S+H358D+A364G+S388K+D470T+S481A;

V45L+A117S+L149R+S214Q+H219V+V258P+A276S+H358D+A364G+S388K+D470T;

V45L+A117S+L149R+S214Q+V258P+A276S+H358D+A364G+S388K+D470T+S481S;

V45L+A117S+L149R+G187K+S214Q+V258P+A276S+A364G+S388K+D470T+V45L+A117S+L149R+S214Q+V258P+A276S+A364G+S388K+N395D+D470T+R483V;

V45L+A117S+L149R+S214Q+V258P+A276S+H358D+A364G+S388K+I469T+D470T+R483V;

V45L+A79C+N127D+L149R+V258P+A276S+A364G+S388K;

D9E+V45L+A79C+L149R+V258P+A276S+A364G+S388K;

V45L+L149R+S214E+V258P+A276S+H358D+A364G+D470T;

V45L+A117S+L149R+G187L+S214Q+V258P+A276S+A364G+S388K+D470T+R483V;

V45L+A117S+L149R+S214Q+V258P+A276S+H358D+A364G+S388K+D470T+N529T+L530R;

V45L+A79C+L149R+W250Y+V258P+A276S+A364G+S388K;

V45L+A117S+L149R+S214Q+V258P+A276S+H358D+A364G+S388K+D470T+V497P;

V45L+A117S+L149R+S214Q+V258P+A276S+H358D+A364G+S388K+D470T+N527H;

V45L+A117S+L149R+S214Q+V258P+A276S+A364G+S388K+D470T+R483V+Q496Q;

V45L+A117S+L149R+N168G+S214Q+V258P+A276S+A364G+S388K+D470T+R483V;

V45L+A117S+L149R+S214Q+V258P+A276S+A364G+S388K+D470T+N476N+R483V;

V45L+A117S+L149R+S214Q+V258P+A276S+A364G+S388K+D470T+T477V+R483V;

V45L+A117S+L149R+S214Q+V258P+A276S+V350L+A364G+S388K+D470T+R483V;

V45L+A117S+L149R+S214Q+V258P+A276S+A364G+S388K+D470T+L478N+R483V;

V45L+A117S+L149R+S214E+V258P+A276S+A364G+S388K+D470T+R483V;

M28P+V45L+A117S+L149R+S214Q+V258P+A276S+H358D+A364G+S388K+D470T;

V45L+A117S+L149R+S214Q+V258P+A276S+A364G+S388K+V393H+D470T+R483V;

V45L+Q61R+A117S+L149R+S214Q+V258P+A276S+A364G+S388K+D470T+R483V;

V45L+A117S+L149R+S214Q+V258P+A276S+A364G+S388K+D470T+R483V+V497P;

V45L+A117S+L149R+S214Q+V258P+A276S+A364G+S388K+D470T+R483V+V498I;

V45L+A117S+L149R+S214Q+V258P+A276S+S282N+H358D+A364G+S388K+D470T+L530R;

V45L+T70R+A117S+L149R+S214Q+V258P+A276S+L281V+H358D+A364G+C375V+S388K+G394A+D470T+R483V;

V45L+A117S+L149R+S214Q+V258P+A276S+A286E+A364G+S388K+D470T+R483V;

V45L+A117S+L149R+S214Q+V258P+A276S+A364G+S388R+D470T+R483V;

V45L+A117S+L149R+S214Q+V258P+A276S+A364G+S388K+D470A+R483V;

V45L+A117S+L149R+S214Q+V258P+A276S+A364G+S388K+D470T+T477K+R483V;

V45L+A117S+L149R+S214Q+V258P+A276S+A364G+S388K+D470T+R483V+Q496E;

V45L+A117S+L149R+S214Q+V258P+A276S+A364G+S388K+D470T+R483V+Q496R;

V45L+A117S+L149R+S214Q+V258P+A276S+A364G+S388K+D470T+R483V+A499L;

V45L+L149R+S214E+V258P+A276S+H358D+A364G+D470T;

V45L+L149R+S214E+V258P+A276S+H358D+A364G+D470T;

V45L+A117S+L149R+S214Q+T215I+V258P+A276S+H358D+A364G+S388K+D470T;

V45L+T70R+A117S+L149R+S214Q+V258P+A276S+L281V+H358D+A364G+S388K+G394A+C468S+D470T+R483V;

V45L+A117S+L149R+S214Q+V258P+A276S+A364G+S388K+Y391V+D470T+R483V;

V45L+A117S+L149R+S214Q+V258P+A276Q+A364G+S388K+D470T+R483V;

V45L+A117S+L149R+S214Q+V258P+A276S+H358D+A364G+S388K+D470T+G503L+L530R;

V45L+A117S+L149R+S214Q+H219I+V258P+A276S+H358D+A364G+S388K+D470T+L530R;

V45L+A117S+L149R+S214Q+V258P+A276S+H358D+A364G+S388K+D470T+N476D;

V45L+A117S+L149R+S214Q+V258P+A276S+H358D+A364G+S388K+V393H+D470T;

V45L+A117S+L149R+S214Q+V258P+A276S+A364G+S388K+D470T+R483V;

V45L+A117S+L149R+S214Q+V258P+A276S+A286S+A364G+S388K+D470T+R483V;

V45L+A117S+L149R+S214Q+V258P+A276S+A364G+S388K+D470T+R483V+S501E;

V45L+A117S+L149R+S214Q+V258P+A276S+A364G+S388K+D470T+R483V+N529T;

V45L+A117S+L149R+S214Q+V258P+A276S+A364G+S388K+D470T+R483V+S501N;

V45L+A117S+L149R+S214Q+V258P+A276S+H358D+A364G+S388K+D470T;

V45L+A117S+L149R+S214Q+V258P+A276S+A364G+S388K+D470T+S481S+R483V;

V45L+A117S+L149R+S214Q+V258P+A276S+A364G+S388K+D470T+L478P+R483V;

D11E+V45L+A117S+L149R+S214Q+V258P+A276S+A364G+S388K+D470T+R483V;

V45L+A117S+L149R+S214Q+V258P+A276S+H358D+A364G+S388K+D470T+T477V;

V45L+A117S+L149R+S214Q+V258P+A276S+H358D+A364G+S388K+D470T+V497P+L530R;

V45L+A117S+L149R+T211H+S214Q+V258P+A276S+H358D+A364G+S388K+D470T;

V45L+A117S+L149R+S214Y+V258P+A276S+H358D+A364G+S388K+D470T+R483V;

V45L+A117S+L149R+S214Q+W250Y+V258P+A276S+A364G+S388K+D470T+R483V;

V45L+S92D+A117S+L149R+S214Q+V258P+A276S+A364G+S388K+D470T+R483V;

V45L+A117S+L149R+S214Q+V258P+A276S+A364G+S388K+D470T+R483V+N527H;

V45N+A117S+L149R+S214Q+V258P+A276S+A364G+S388K+D470T+R483V;

V45L+A117S+L149R+S214Q+V258P+A276S+H358D+A364G+Q385C+S388K+D470T;

V45L+A117S+L149R+S173Y+S214Q+V258P+A276S+A364G+S388K+D470T+R483V;

V45L+A117S+L149R+S214Q+V258P+A276S+A364G+S388H+D470T+R483V;

V45L+A117S+L149R+S214Q+V258P+A276S+A364G+S388K+S389H+D470T+V45L+A117S+L149R+S214Q+H219V+V258P+A276S+A364G+S388K+D470T+R483V;

V45L+A79C+A117S+L149R+S214Q+V258P+A276S+A364G+S388K+D470T;

V45L+A117S+L149R+S214Q+T215I+V258P+A276S+H358D+A364G+S388K+D470T+L530R;

V45L+A117S+L149R+S214Q+V258P+A276S+H358D+A364G+S388K+D470T+Q496E;

V45L+A117S+L149R+A170S+S214Q+V258P+A276S+H358D+A364G+S388K+D470T;

V45L+A117S+L149R+S214Q+V258P+A276S+H358D+A364G+S388K+S389V+D470T+R483V;

V45L+A117S+L149R+S214Q+V258P+A276S+H358D+A364G+S388K+S389V+D470T+L530R;

V45L+A117S+L149R+S214Q+V258P+A276S+H358D+A364G+S388A+D470T;

V45L+A117S+L149R+S214Q+V258P+A276S+H358D+A364G+S388K+S389V+D470T;

V45L+T70R+A117S+L149R+S214Q+V258P+A276S+H358D+A364G+S388K+D470T;

V45L+A117S+L149R+S214Q+H219I+V258P+A276S+A364G+S388K+D470T+R483V;

V45L+A79C+L149R+N163H+V258P+A276S+A364G+S388K;

V45L+A117S+L149R+S214Q+V258P+A276S+A364G+S388K+D470T+R483V+A499Y;

V45L+A117S+L149R+S214Q+V258P+A276S+L281V+A364G+S388K+D470T+R483V;

V45L+A117S+L149R+S214Q+V258P+A276S+A364G+S388K+D470T+R483V+V500T;

V45L+A117S+L149R+T180Q+S214Q+V258P+A276S+H358D+A364G+S388K+D470T;

V45L+A117S+L149R+S214Q+V258P+A276S+H358D+A364G+S388K+D470T+V500V;

V45L+A117S+L149R+S214Q+V258P+A276S+H358D+A364G+S388K+D470T+G526T;

V45L+C60L+T70R+A117S+L149R+S214Q+V258P+A276S+L281V+H358D+A364G+S388K+G394A+D470T+R483V;

V45L+C69L+T70R+A117S+L149R+S214Q+V258P+A276S+L281V+H358D+A364G+S388K+G394A+D470T+R483V;

V45L+A117S+L149R+S214Q+V258P+A276S+A364G+S388A+D470T+R483V;

V45L+A117S+L149R+S214Q+V258P+A276S+A286S+H358D+A364G+S388K+D470T;

V45L+A117S+L149R+S214Q+V258P+A276S+H358D+A364G+S388K+D470T+R483V+V497D;

V45L+A117S+L149R+S214Q+V258P+A276S+H358D+A364G+S388K+D470A+L530R;

V45L+A117S+L149R+S214Q+V258P+A276S+A364G+S388K+D470T+R483V+D525L;

V45L+A117S+L149R+S214Q+V258P+A276S+H358D+A364G+S388K+D470T+N476N;

V45L+A117S+L149R+S214Q+V258P+A276S+L281V+A364G+S388K+D470T;

V45L+A117S+L149R+S214Q+V258P+A276S+A364G+Q384A+S388K+D470T;

V45L+A117S+L149R+S214Q+V258P+A276S+A286E+A364G+S388K+D470T;

V45L+A117S+L149R+S214Q+V258P+A276S+A364G+S388K+D470T+T477K;

V45L+A117S+L149R+S214Q+V258P+A276S+H358D+A364G+S388K+S389H+D470T;

V45L+A117S+L149R+S214Y+V258P+A276S+H358D+A364G+S388K+D470T+L530R;

V45L+A117S+L149R+S214Y+V258P+A276S+H358D+A364G+S388K+D470T;

V45L+A117S+L149R+S214Q+V258P+A276S+A364G+S388K+G394A+D470T;

V45L+A117S+L149R+S214Q+V258P+A276S+A364G+S388K+D470T+N476D;

V45L+A117S+L149R+S214Q+V258P+A276S+H358D+A364G+S388K+D470T+L478N+R483V;

V45L+C60A+T70R+A117S+L149R+S214Q+V258P+A276S+L281V+H358D+A364G+S388K+G394A+D470T+R483V;

V45L+A117S+L149R+S214Q+V258P+A276S+A364G+S388K+V393H+D470T;

V45L+A117S+L149R+S214Q+V258P+A276S+A364G+S388K+N395D+D470T;

A25W+V45L+A117S+L149R+S214Q+V258P+A276S+H358D+A364G+S388K+D470T;

V45L+T70R+A117S+L149R+S214Q+C243L+V258P+A276S+L281V+H358D+A364G+S388K+G394A+D470T+R483V;

V45L+A117S+L149R+S214Q+V258P+A276S+H358T+A364G+S388K+D470T;

V45L+A117S+L149R+S214Q+V258P+A276S+A364G+S388K+D470T+L478N;

V45L+C60S+T70R+A117S+L149R+S214Q+V258P+A276S+L281V+H358D+A364G+S388K+G394A+D470T+R483V;

V45L+A117S+L149R+S214Q+W250Y+V258P+A276S+A364G+S388K+D470T;

V45L+A117S+L149R+S214Q+T215V+V258P+A276S+A364G+S388K+D470T+R483V;

V45L+Q61R+A117S+L149R+S214Q+V258P+A276S+H358D+A364G+S388K+D470T;

V45L+A117S+L149R+G187L+S214Q+V258P+A276S+A364G+S388K+D470T;

D9E+V45L+A117S+L149R+S214Q+V258P+A276S+A364G+S388K+D470T;

V45L+A117S+L149R+S214Q+V258P+A276S+H358D+A364G;

V45L+C69A+T70R+A117S+L149R+S214Q+V258P+A276S+L281V+H358D+A364G+S388K+G394A+D470T+R483V;

V45L+T70R+A117S+L149R+C161S+S214Q+V258P+A276S+L281V+H358D+A364G+S388K+G394A+D470T+R483V;

V45L+A117S+L149R+S214Q+H219I+V258P+A276S+H358D+A364G+S388K+D470T;

V45L+A117S+L149R+S214Q+V258P+A276S+S282N+A364G+S388K+D470T;

V45L+A117S+L149R+T211H+S214Q+V258P+A276S+A364G+S388K+D470T+R483V;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

D11E+V45L+A117S+L149R+S214Q+V258P+A276S+A364G+S388K+D470T;

V45L+A117S+L149R+S214Q+V258P+A276S+A364G+S388K+D470T+N476N;

V45L+A117S+L149R+S214Q+V258P+A276S+A364G+S388K+G394D+D470T;

V45L+Q61K+A117S+L149R+S214Q+V258P+A276S+A364G+S388K+D470T;

V45L+A117S+L149R+S214Q+V258P+A276S+H358D+A364G+S388K+D470T+G503L;

V45L+A117S+L149R+S214Q+V258P+A276S+A286S+A364G+S388K+D470T;

V45L+A117S+L149R+S214Q+V258P+A276S+H358D+A364G+S388K+D470A;

V45L+A117S+L149R+S214Q+V258P+A276S+A364G+S388K+G394Q+D470T;

V45L+A117S+L149R+S214Q+V258P+A276S+A364G+S388R+D470T;

V45L+A117S+L149R+S214Q+V258P+A276S+A364G+S388K+D470T+L478P;

V45L+A117S+L149R+S214Q+V258P+A276S+V350L+A364G+S388K+D470T;

V45L+D113T+A117S+L149R+S214Q+V258P+A276S+A364G+S388K+D470T;

V45L+A117S+L149R+T180Q+S214Q+V258P+A276S+A364G+S388K+D470T;

V45L+C69V+T70R+A117S+L149R+S214Q+V258P+A276S+L281V+H358D+A364G+S388K+G394A+D470T+R483V;

V45L+A117S+L149R+S214Q+T215V+V258P+A276S+A364G+S388K+D470T+D525L;

V45L+A117S+L149R+S214Q+V258P+A276S+A364G+S388K+D470T;

V45L+A117S+L149R+S214Q+V258P+A276S+H358D+A364G+S388K+D470T+V500T;

V45L+A117S+L149R+S214Q+V258P+A276S+A364G+S388K+S389H+D470T;

V45L+A117S+L149R+S214Q+V258P+A276S+A364G+S388K+V393R+D470T;

V45L+A117S+L149R+S214Q+V258P+A276S+A364G+S388K+D470T+Q496R;

V45L+A117S+N127D+L149R+S214Q+V258P+A276S+A364G+S388K+D470T;

V45L+A117S+L149R+S214Q+V258P+A276S+A364G+S388K+D470T+A499Y;

V45L+A117S+L149R+S214Q+T215V+V258P+A276S+H358D+A364G+S388K+D470T;

V45L+A117S+L149R+S214Q+V258P+A276S+H358D+A364G+S388K+D470T+T480L;

V45L+A117S+L149R+S214Q+V258P+A276S+A364G+S388L+D470T;

V45L+A117S+L149R+S173Y+S214Q+V258P+A276S+A364G+S388K+D470T;

V45L+S92D+A117S+L149R+S214Q+V258P+A276S+A364G+S388K+D470T;

V45L+A117S+L149R+S214Q+V258P+A276S+A364G+S388K+S389V+D470T+R483V;

V45L+A117S+L149R+S214Q+V258P+A276S+A364G+S388K+D470T+N529T;

V45L+C60V+T70R+A117S+L149R+S214Q+V258P+A276S+L281V+H358D+A364G+S388K+G394A+D470T+R483V;

V45L+A117S+L149R+S214Q+V258P+A276S+A364G+S388K+D470T+R483V+V497E;

V45L+A117S+L149R+S214Q+V258P+A276S+H358D+A364G+S388K+L478N;

V45L+T70R+A117S+L149R+S214Q+V258P+A276S+A364G+S388K+D470T;

M28P+V45L+A117S+L149R+S214Q+V258P+A276S+A364G+S388K+D470T;

V45L+A117S+L149R+S214Q+V258P+A276Q+A364G+S388K+D470T;

V45L+A117S+L149R+S214Q+V258P+A276S+H358D+A364G+S388K+D470V+R483V;

V45L+A117S+L149R+S214Y+V258P+A276S+A364G+S388K+D470T+R483V;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by TSA of at least 10° C. compared to the xylanase of SEQ ID NO: 1.

In one aspect, the present invention relates to xylanase variants comprises at least one of the following substitutions or combinations of substitutions:

V45L+Q61K+T70R+A117S+L149R+S214Q+V258P+A276S+L281V+A286E+H358D+A3 64G+Q384A+S388K+V393H+D470T+R483V;

V45L+Q61K+T70R+A117S+L149R+S214Q+V258P+A276S+L281V+H358D+A364G+Q3 84A+S388K+G394Q+D470T+R483V;

V45L+Q61K+T70R+A117S+L149R+S214Q+V258P+A276S+L281V+H358D+A364G+Q3 84A+S388K+G394A+D470T+R483V;

V45L+Q61K+T70R+A117S+L149R+S214Q+V258P+A276S+L281V+H358D+A364G+Q3 84A+S388K+V393H+D470T+R483V;

V45L+Q61K+T70R+A117S+L149R+S214Q+V258P+A276S+L281V+H358D+A364G+Q3 84A+S388K+V393H+D470T+R483V;

V45L+Q61K+T70R+A117S+L149R+S214Q+V258P+A276S+L281V+H358D+A364G+Q3 84A+S388K+G394A+D470T+R483V;

V45L+Q61K+T70R+A117S+L149R+S214Q+V258P+A276S+L281V+H358D+A364G+Q3 84A+S388K+V393H+D470T+R483V;

V45L+Q61K+T70R+A117S+L149R+S214Q+V258P+A276S+L281V+H358D+A364G+Q3 84A+S388K+G394A+D470T+R483V;

V45L+Q61K+T70R+A117S+L149R+S214Q+V258P+A276S+L281V+A286E+H358D+A3 64G+Q384A+S388K+V393H+D470T+R483V;

V45L+Q61K+T70R+A117S+L149R+S214Q+V258P+A276S+L281V+H358D+A364G+Q3 84A+S388K+V393H+D470T+R483V;

V45L+Q61K+T70R+A117S+L149R+S214Q+V258P+A276S+L281V+H358D+A364G+Q3 84A+S388K+G394A+D470T+R483V;

V45L+A79C+A117S+L149R+S214Q+V258P+A276S+H358D+A364G+S388K+D470T+L 530R;

V45L+A79C+A117S+L149R+S214Q+V258P+A276S+H358D+A364G+S388K+D470T+R 483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+Q61K+A117S+L149R+S214Q+V258P+A276S+L281V+H358D+A364G+S388K+G 394A+D470T+R483V+L530R;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+Q61K+A117S+L149R+S214Q+V258P+A276S+L281V+H358D+A364G+S388K+G 394A+D470T+L530R;

V45L+Q61K+A117S+L149R+S214Q+V258P+A276S+L281V+H358D+A364G+S388K+G 394A+D470T+R483V+L530R;

V45L+Q61K+A117S+L149R+S214Q+V258P+A276S+L281V+H358D+A364G+S388K+G 394A+D470T+R483V+L530R;

V45L+Q61K+A117S+L149R+S214Q+V258P+A276S+L281V+H358D+A364G+S388K+D 470T+R483V+L530R;

V45L+Q61K+A117S+L149R+S214Q+V258P+A276S+L281V+H358D+A364G+S388K+G 394A+D470T+R483V+L530R;

V45L+Q61K+A117S+L149R+S214Q+V258P+A276S+L281V+H358D+A364G+S388K+D 470T+R483V+L530R;

V45L+A117S+L149R+G187K+S214Q+V258P+A276S+A286E+H358D+A364G+Q384A+S388K+V393H+D470T+R483V;

V45L+A117S+L149R+G187K+S214Q+V258P+A276S+A286E+H358D+A364G+Q384A+S388K+N395D+D470T+R483V; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by TSA of at least 15° C. compared to the xylanase of SEQ ID NO: 1.

In one aspect, the present invention relates to xylanase variants comprises at least one of the following substitutions or combinations of substitutions:

V45L+Q61K+T70R+A117S+L149R+S214Q+V258P+A276S+L281V+A286E+H358D+A3 64G+Q384A+S388K+V393H+D470T+R483V;

V45L+Q61K+T70R+A117S+L149R+S214Q+V258P+A276S+L281V+H358D+A364G+Q3 84A+S388K+G394Q+D470T+R483V;

V45L+Q61K+T70R+A117S+L149R+S214Q+V258P+A276S+L281V+H358D+A364G+Q3 84A+S388K+G394A+D470T+R483V;

V45L+Q61K+T70R+A117S+L149R+S214Q+V258P+A276S+L281V+H358D+A364G+Q3 84A+S388K+V393H+D470T+R483V;

V45L+Q61K+T70R+A117S+L149R+S214Q+V258P+A276S+L281V+H358D+A364G+Q3 84A+S388K+V393H+D470T+R483V;

V45L+Q61K+T70R+A117S+L149R+S214Q+V258P+A276S+L281V+H358D+A364G+Q3 84A+S388K+G394A+D470T+R483V;

V45L+Q61K+T70R+A117S+L149R+S214Q+V258P+A276S+L281V+H358D+A364G+Q3 84A+S388K+V393H+D470T+R483V;

V45L+Q61K+T70R+A117S+L149R+S214Q+V258P+A276S+L281V+H358D+A364G+Q3 84A+S388K+G394A+D470T+R483V;

V45L+Q61K+T70R+A117S+L149R+S214Q+V258P+A276S+L281V+A286E+H358D+A3 64G+Q384A+S388K+V393H+D470T+R483V;

V45L+Q61K+T70R+A117S+L149R+S214Q+V258P+A276S+L281V+H358D+A364G+Q3 84A+S388K+V393H+D470T+R483V;

V45L+Q61K+T70R+A117S+L149R+S214Q+V258P+A276S+L281V+H358D+A364G+Q3 84A+S388K+G394A+D470T+R483V;

V45L+A79C+A117S+L149R+S214Q+V258P+A276S+H358D+A364G+S388K+D470T+L 530R;

V45L+A79C+A117S+L149R+S214Q+V258P+A276S+H358D+A364G+S388K+D470T+R 483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by TSA of at least 16° C. compared to the xylanase of SEQ ID NO: 1.

In one aspect, the present invention relates to xylanase variants comprises at least one of the following substitutions or combinations of substitutions:

V45L+Q61K+T70R+A117S+L149R+S214Q+V258P+A276S+L281V+A286E+H358D+A3 64G+Q384A+S388K+V393H+D470T+R483V;

V45L+Q61K+T70R+A117S+L149R+S214Q+V258P+A276S+L281V+H358D+A364G+Q3 84A+S388K+G394Q+D470T+R483V;

V45L+Q61K+T70R+A117S+L149R+S214Q+V258P+A276S+L281V+H358D+A364G+Q3 84A+S388K+G394A+D470T+R483V;

V45L+Q61K+T70R+A117S+L149R+S214Q+V258P+A276S+L281V+H358D+A364G+Q3 84A+S388K+V393H+D470T+R483V; and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by TSA of at least 17° C. compared to the xylanase of SEQ ID NO: 1.

In one aspect, the present invention relates to xylanase variants comprises at least one of the following substitutions or combinations of substitutions:

V45L+A79C+A117S+L149R+S214Q+V258P+A276S+A364G+S388K+D470T+V45L+A79C+A117S+L149R+S214Q+V258P+A276S+H358D+A364G+S388K+D470T;

V45L+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+V258P+A276S+A364G+Q384A+S388K+D470T+R483V;

V45L+A79C+A117S+L149R+S214Q+V258P+A276S+H358D+A364G+S388K;

V45L+A117S+L149R+S214Q+V258P+A276S+A364G+S388K+D470T+R483V;

V45L+A117S+L149R+S214Q+V258P+A276S+H358D+A364G+S388K+D470T;

V45L+A79C+A117S+L149R+S214Q+V258P+A276S+A364G+S388K+D470T;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

V45L+A117S+L149R+S214Q+V258P+A276S+H358D+A364G+S388K+D470A;

V45L+A117S+L149R+S214Q+V258P+A276S+H358D+A364G+S388K+L478N;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

D11E+V45L+A117S+L149R+S214Q+V258P+A276S+H358D+A364G+S388K;

V45L+L149R+V258P+A276S+H358T+A364G;

A25W+V45L+L149R+V258P+A276S+A364G;

V45L+L149R+S214Q+V258P+A276S+A364G+S388K;

V45L+L149R+V258P+A276S+L281V+A364G;

V45L+N127D+L149R+V258P+A276S+A364G+S388K;

V45L+L149R+V258P+A276S+H358T+A364G+S388K;

V45L+L149R+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

V45L+L149R+S214Q+V258P+A276S+H358T+A364G;

A25W+V45L+L149R+V258P+A276Q+A364G;

V45L+L149R+V258P+A276S+A364G;

A25W+V45L+L149R+W250Y+A276Q+A364G;

A25W+V45L+L149R+A276Q+L281V+A364G;

A25W+V45L+L149R+A276Q+A286E+A364G;

A25W+V45L+L149R+A276Q+A364G+S388K;

V45L+L149R+V258P+L281V+A364G;

A25W+V45L+L149R+A276Q+A364G;

V45L+L149R+V258P+A364G;

V45L+L149R+V258P+S282N+A364G;

V45L+L149R+A276Q+A364G;

V45L+L149R+A276S+A364G;

V45L+L149R;

V45L+L149R+A364G;

V45L+L149R+V258R+A364G;

V258P+A276Q;

V258P+A276S;

V45L+A276S;

V45L+A276Q;

L149R+W250Y;

V258P+S388K;

A276Q+S388K;

W250Y+A276Q;

A276S+S388K;

A276Q+L281V;

W250Y+A276S;

A276S+L281V;

G187S+A276Q;

V45L+L281V;

V45L;

L149R;

A79C;

V45N;

V45L+D11E;

L149R+S388K;

L149R+D11E;

A364G+D11E;

S388K+D11E; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by n-DSF of at least 0.5° C. compared to the xylanase of SEQ ID NO: 1.

In one aspect, the present invention relates to xylanase variants comprises at least one of the following substitutions or combinations of substitutions:

V45L+A79C+A117S+L149R+S214Q+V258P+A276S+A364G+S388K+D470T+R483V;

V45L+A79C+A117S+L149R+S214Q+V258P+A276S+H358D+A364G+S388K+D470T;

V45L+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+V258P+A276S+A364G+Q384A+S388K+D470T+R483V;

V45L+A79C+A117S+L149R+S214Q+V258P+A276S+H358D+A364G+S388K;

V45L+A117S+L149R+S214Q+V258P+A276S+A364G+S388K+D470T+R483V;

V45L+A117S+L149R+S214Q+V258P+A276S+H358D+A364G+S388K+D470T;

V45L+A79C+A117S+L149R+S214Q+V258P+A276S+A364G+S388K+D470T;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

V45L+A117S+L149R+S214Q+V258P+A276S+H358D+A364G+S388K+D470A;

V45L+A117S+L149R+S214Q+V258P+A276S+H358D+A364G+S388K+L478N;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

D11E+V45L+A117S+L149R+S214Q+V258P+A276S+H358D+A364G+S388K;

V45L+L149R+V258P+A276S+H358T+A364G;

A25W+V45L+L149R+V258P+A276S+A364G;

V45L+L149R+S214Q+V258P+A276S+A364G+S388K;

V45L+L149R+V258P+A276S+L281V+A364G;

V45L+N127D+L149R+V258P+A276S+A364G+S388K;

V45L+L149R+V258P+A276S+H358T+A364G+S388K;

V45L+L149R+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

V45L+L149R+S214Q+V258P+A276S+H358T+A364G;

A25W+V45L+L149R+V258P+A276Q+A364G;

V45L+L149R+V258P+A276S+A364G;

A25W+V45L+L149R+W250Y+A276Q+A364G;

A25W+V45L+L149R+A276Q+L281V+A364G;

A25W+V45L+L149R+A276Q+A286E+A364G;

A25W+V45L+L149R+A276Q+A364G+S388K;

V45L+L149R+V258P+L281V+A364G;

A25W+V45L+L149R+A276Q+A364G;

V45L+L149R+V258P+A364G;

V45L+L149R+V258P+S282N+A364G;

V45L+L149R+A276Q+A364G;

V45L+L149R+A276S+A364G;

V45L+L149R;

V45L+L149R+A364G;

V45L+L149R+V258R+A364G;

V258P+A276Q;

V258P+A276S;

V45L+A276S;

V45L+A276Q;

L149R+W250Y;

V258P+S388K;

A276Q+S388K;

W250Y+A276Q;

A276S+S388K;

A276Q+L281V;

W250Y+A276S;

A276S+L281V;

G187S+A276Q;

V45L+L281V;

L149R;

A79C;

V45N;

V45L+D11E;

L149R+S388K;

L149R+D11E;

S388K+D11E; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by n-DSF of at least 2.0° C. compared to the xylanase of SEQ ID NO: 1.

In one aspect, the present invention relates to xylanase variants comprises at least one of the following substitutions or combinations of substitutions:

V45L+A79C+A117S+L149R+S214Q+V258P+A276S+A364G+S388K+D470T+R483V;

V45L+A79C+A117S+L149R+S214Q+V258P+A276S+H358D+A364G+S388K+D470T;

V45L+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A117S+L149R+S214Q+V258P+A276S+A364G+S388K+G394A+D470T+V45L+A93T+A117S+L149R+S214Q+V258P+A276S+H358D+A364G+S388K+D470T+A499Y;

V45L+A117S+L149R+S214Q+V258P+A276S+A364G+Q384A+S388K+D470T+R483V;

V45L+A79C+A117S+L149R+S214Q+V258P+A276S+H358D+A364G+S388K;

V45L+A117S+L149R+S214Q+V258P+A276S+A364G+S388K+D470T+R483V;

V45L+A117S+L149R+S214Q+V258P+A276S+H358D+A364G+S388K+D470T;

V45L+A79C+A117S+L149R+S214Q+V258P+A276S+A364G+S388K+D470T;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

V45L+A117S+L149R+S214Q+V258P+A276S+H358D+A364G+S388K+D470A;

V45L+A117S+L149R+S214Q+V258P+A276S+H358D+A364G+S388K+L478N;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

D11E+V45L+A117S+L149R+S214Q+V258P+A276S+H358D+A364G+S388K;

V45L+L149R+V258P+A276S+H358T+A364G;

A25W+V45L+L149R+V258P+A276S+A364G;

V45L+L149R+S214Q+V258P+A276S+A364G+S388K;

V45L+L149R+V258P+A276S+L281V+A364G;

V45L+N127D+L149R+V258P+A276S+A364G+S388K;

V45L+L149R+V258P+A276S+H358T+A364G+S388K;

V45L+L149R+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

V45L+L149R+S214Q+V258P+A276S+H358T+A364G;

A25W+V45L+L149R+V258P+A276Q+A364G;

V45L+L149R+V258P+A276S+A364G;

A25W+V45L+L149R+W250Y+A276Q+A364G;

A25W+V45L+L149R+A276Q+L281V+A364G;

A25W+V45L+L149R+A276Q+A286E+A364G;

A25W+V45L+L149R+A276Q+A364G+S388K;

V45L+L149R+V258P+L281V+A364G;

A25W+V45L+L149R+A276Q+A364G;

V45L+L149R+V258P+A364G;

V45L+L149R+V258P+S282N+A364G;

V45L+L149R+A276Q+A364G;

V45L+L149R+A276S+A364G;

V45L+L149R;

V45L+L149R+A364G;

V45L+L149R+V258R+A364G;

V258P+A276Q;

V258P+A276S;

V45L+A276S;

V45L+A276Q;

L149R+W250Y;

V258P+S388K;

A276Q+S388K;

W250Y+A276Q;

A276S+S388K;

A276Q+L281V;

W250Y+A276S;

A276S+L281V;

G187S+A276Q;

A79C;

V45L+D11E;

L149R+S388K;

L149R+D11E; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by n-DSF of at least 2.5° C. compared to the xylanase of SEQ ID NO: 1.

In one aspect, the present invention relates to xylanase variants comprises at least one of the following substitutions or combinations of substitutions:

V45L+A79C+A117S+L149R+S214Q+V258P+A276S+A364G+S388K+D470T+R483V;

V45L+A79C+A117S+L149R+S214Q+V258P+A276S+H358D+A364G+S388K+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+V258P+A276S+A364G+Q384A+S388K+D470T+R483V;

V45L+A79C+A117S+L149R+S214Q+V258P+A276S+H358D+A364G+S388K;

V45L+A117S+L149R+S214Q+V258P+A276S+A364G+S388K+D470T+R483V;

V45L+A117S+L149R+S214Q+V258P+A276S+H358D+A364G+S388K+D470T;

V45L+A79C+A117S+L149R+S214Q+V258P+A276S+A364G+S388K+D470T;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

V45L+A117S+L149R+S214Q+V258P+A276S+H358D+A364G+S388K+D470A;

V45L+A117S+L149R+S214Q+V258P+A276S+H358D+A364G+S388K+L478N;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

D11E+V45L+A117S+L149R+S214Q+V258P+A276S+H358D+A364G+S388K;

V45L+L149R+V258P+A276S+H358T+A364G;

A25W+V45L+L149R+V258P+A276S+A364G;

V45L+L149R+S214Q+V258P+A276S+A364G+S388K;

V45L+L149R+V258P+A276S+L281V+A364G;

V45L+N127D+L149R+V258P+A276S+A364G+S388K;

V45L+L149R+V258P+A276S+H358T+A364G+S388K;

V45L+L149R+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

V45L+L149R+S214Q+V258P+A276S+H358T+A364G;

A25W+V45L+L149R+V258P+A276Q+A364G;

V45L+L149R+V258P+A276S+A364G;

A25W+V45L+L149R+W250Y+A276Q+A364G;

A25W+V45L+L149R+A276Q+L281V+A364G;

A25W+V45L+L149R+A276Q+A286E+A364G;

A25W+V45L+L149R+A276Q+A364G+S388K;

V45L+L149R+V258P+L281V+A364G;

A25W+V45L+L149R+A276Q+A364G;

V45L+L149R+V258P+A364G;

V45L+L149R+V258P+S282N+A364G;

V45L+L149R+A276Q+A364G;

V45L+L149R+A276S+A364G;

V45L+L149R;

V45L+L149R+A364G;

V45L+L149R+V258R+A364G;

V258P+A276Q;

V258P+A276S;

V45L+A276S;

V45L+A276Q;

L149R+W250Y;

V258P+S388K;

A276Q+S388K;

W250Y+A276Q;

A276S+S388K;

A276Q+L281V;

W250Y+A276S;

A276S+L281V;

L149R+S388K; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by n-DSF of at least 3.0° C. compared to the xylanase of SEQ ID NO: 1.

In one aspect, the present invention relates to xylanase variants comprises at least one of the following substitutions or combinations of substitutions:

V45L+A79C+A117S+L149R+S214Q+V258P+A276S+A364G+S388K+D470T+R483V;

V45L+A79C+A117S+L149R+S214Q+V258P+A276S+H358D+A364G+S388K+D470T;

V45L+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+V258P+A276S+A364G+Q384A+S388K+D470T+R483V;

V45L+A79C+A117S+L149R+S214Q+V258P+A276S+H358D+A364G+S388K;

V45L+A117S+L149R+S214Q+V258P+A276S+A364G+S388K+D470T+R483V;

V45L+A117S+L149R+S214Q+V258P+A276S+H358D+A364G+S388K+D470T;

V45L+A79C+A117S+L149R+S214Q+V258P+A276S+A364G+S388K+D470T;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

V45L+A117S+L149R+S214Q+V258P+A276S+H358D+A364G+S388K+D470A;

V45L+A117S+L149R+S214Q+V258P+A276S+H358D+A364G+S388K+L478N;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

D11E+V45L+A117S+L149R+S214Q+V258P+A276S+H358D+A364G+S388K;

V45L+L149R+V258P+A276S+H358T+A364G;

A25W+V45L+L149R+V258P+A276S+A364G;

V45L+L149R+S214Q+V258P+A276S+A364G+S388K;

V45L+L149R+V258P+A276S+L281V+A364G;

V45L+N127D+L149R+V258P+A276S+A364G+S388K;

V45L+L149R+V258P+A276S+H358T+A364G+S388K;

V45L+L149R+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

V45L+L149R+S214Q+V258P+A276S+H358T+A364G;

A25W+V45L+L149R+V258P+A276Q+A364G;

V45L+L149R+V258P+A276S+A364G;

A25W+V45L+L149R+W250Y+A276Q+A364G;

A25W+V45L+L149R+A276Q+L281V+A364G;

A25W+V45L+L149R+A276Q+A286E+A364G;

A25W+V45L+L149R+A276Q+A364G+S388K;

V45L+L149R+V258P+L281V+A364G;

A25W+V45L+L149R+A276Q+A364G;

V45L+L149R+V258P+A364G;

V45L+L149R+V258P+S282N+A364G;

V45L+L149R+A276Q+A364G;

V45L+L149R+A276S+A364G;

V45L+L149R;

V45L+L149R+A364G;

V45L+L149R+V258R+A364G;

V258P+A276Q;

V258P+A276S;

V45L+A276S;

V45L+A276Q;

V258P+S388K;

A276Q+S388K;

W250Y+A276Q;

A276S+S388K;

A276Q+L281V;

L149R+S388K; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by n-DSF of at least 3.5° C. compared to the xylanase of SEQ ID NO: 1.

In one aspect, the present invention relates to xylanase variants comprises at least one of the following substitutions or combinations of substitutions:

V45L+A79C+A117S+L149R+S214Q+V258P+A276S+A364G+S388K+D470T+R483V;

V45L+A79C+A117S+L149R+S214Q+V258P+A276S+H358D+A364G+S388K+D470T;

V45L+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+V258P+A276S+A364G+Q384A+S388K+D470T+R483V;

V45L+A79C+A117S+L149R+S214Q+V258P+A276S+H358D+A364G+S388K;

V45L+A117S+L149R+S214Q+V258P+A276S+A364G+S388K+D470T+R483V;

V45L+A117S+L149R+S214Q+V258P+A276S+H358D+A364G+S388K+D470T;

V45L+A79C+A117S+L149R+S214Q+V258P+A276S+A364G+S388K+D470T;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

V45L+A117S+L149R+S214Q+V258P+A276S+H358D+A364G+S388K+D470A;

V45L+A117S+L149R+S214Q+V258P+A276S+H358D+A364G+S388K+L478N;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

D11E+V45L+A117S+L149R+S214Q+V258P+A276S+H358D+A364G+S388K;

V45L+L149R+V258P+A276S+H358T+A364G;

A25W+V45L+L149R+V258P+A276S+A364G;

V45L+L149R+S214Q+V258P+A276S+A364G+S388K;

V45L+L149R+V258P+A276S+L281V+A364G;

V45L+N127D+L149R+V258P+A276S+A364G+S388K;

V45L+L149R+V258P+A276S+H358T+A364G+S388K;

V45L+L149R+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

V45L+L149R+S214Q+V258P+A276S+H358T+A364G;

A25W+V45L+L149R+V258P+A276Q+A364G;

V45L+L149R+V258P+A276S+A364G;

A25W+V45L+L149R+W250Y+A276Q+A364G;

A25W+V45L+L149R+A276Q+L281V+A364G;

A25W+V45L+L149R+A276Q+A286E+A364G;

A25W+V45L+L149R+A276Q+A364G+S388K;

V45L+L149R+V258P+L281V+A364G;

A25W+V45L+L149R+A276Q+A364G;

V45L+L149R+V258P+A364G;

V45L+L149R+V258P+S282N+A364G;

V45L+L149R+A276Q+A364G;

V45L+L149R+A276S+A364G;

V45L+L149R;

V45L+L149R+A364G;

V45L+L149R+V258R+A364G;

V45L+A276Q;

V258P+A276Q;

V258P+A276S;

V45L+A276S; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by n-DSF of at least 4.0° C. compared to the xylanase of SEQ ID NO: 1.

In one aspect, the present invention relates to xylanase variants comprises at least one of the following substitutions or combinations of substitutions:

V45L+A79C+A117S+L149R+S214Q+V258P+A276S+A364G+S388K+D470T+V45L+A79C+A117S+L149R+S214Q+V258P+A276S+H358D+A364G+S388K+D470T;

V45L+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+V258P+A276S+A364G+Q384A+S388K+D470T+R483V;

V45L+A79C+A117S+L149R+S214Q+V258P+A276S+H358D+A364G+S388K;

V45L+A117S+L149R+S214Q+V258P+A276S+A364G+S388K+D470T+R483V;

V45L+A117S+L149R+S214Q+V258P+A276S+H358D+A364G+S388K+D470T;

V45L+A79C+A117S+L149R+S214Q+V258P+A276S+A364G+S388K+D470T;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

V45L+A117S+L149R+S214Q+V258P+A276S+H358D+A364G+S388K+D470A;

V45L+A117S+L149R+S214Q+V258P+A276S+H358D+A364G+S388K+L478N;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

D11E+V45L+A117S+L149R+S214Q+V258P+A276S+H358D+A364G+S388K;

V45L+L149R+V258P+A276S+H358T+A364G;

A25W+V45L+L149R+V258P+A276S+A364G;

V45L+L149R+S214Q+V258P+A276S+A364G+S388K;

V45L+L149R+V258P+A276S+L281V+A364G;

V45L+N127D+L149R+V258P+A276S+A364G+S388K;

V45L+L149R+V258P+A276S+H358T+A364G+S388K;

V45L+L149R+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

V45L+L149R+S214Q+V258P+A276S+H358T+A364G;

A25W+V45L+L149R+V258P+A276Q+A364G;

V45L+L149R+V258P+A276S+A364G;

A25W+V45L+L149R+W250Y+A276Q+A364G;

A25W+V45L+L149R+A276Q+L281V+A364G;

A25W+V45L+L149R+A276Q+A286E+A364G;

A25W+V45L+L149R+A276Q+A364G+S388K;

V45L+L149R+V258P+L281V+A364G;

A25W+V45L+L149R+A276Q+A364G;

V45L+L149R+V258P+A364G;

V45L+L149R+V258P+S282N+A364G;

V45L+L149R+A276Q+A364G;

V45L+L149R+A276S+A364G;

V45L+L149R;

V45L+L149R+A364G;

V45L+L149R+V258R+A364G;

V45L+A276Q; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by n-DSF of at least 4.5° C. compared to the xylanase of SEQ ID NO: 1.

In one aspect, the present invention relates to xylanase variants comprises at least one of the following substitutions or combinations of substitutions:

V45L+A79C+A117S+L149R+S214Q+V258P+A276S+A364G+S388K+D470T+R483V;

V45L+A79C+A117S+L149R+S214Q+V258P+A276S+H358D+A364G+S388K+D470T;

V45L+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+V258P+A276S+A364G+Q384A+S388K+D470T+R483V;

V45L+A79C+A117S+L149R+S214Q+V258P+A276S+H358D+A364G+S388K;

V45L+A117S+L149R+S214Q+V258P+A276S+A364G+S388K+D470T+R483V;

V45L+A117S+L149R+S214Q+V258P+A276S+H358D+A364G+S388K+D470T;

V45L+A79C+A117S+L149R+S214Q+V258P+A276S+A364G+S388K+D470T;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

V45L+A117S+L149R+S214Q+V258P+A276S+H358D+A364G+S388K+D470A;

V45L+A117S+L149R+S214Q+V258P+A276S+H358D+A364G+S388K+L478N;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

D11E+V45L+A117S+L149R+S214Q+V258P+A276S+H358D+A364G+S388K;

V45L+L149R+V258P+A276S+H358T+A364G;

A25W+V45L+L149R+V258P+A276S+A364G;

V45L+L149R+S214Q+V258P+A276S+A364G+S388K;

V45L+L149R+V258P+A276S+L281V+A364G;

V45L+N127D+L149R+V258P+A276S+A364G+S388K;

V45L+L149R+V258P+A276S+H358T+A364G+S388K;

V45L+L149R+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

V45L+L149R+S214Q+V258P+A276S+H358T+A364G;

A25W+V45L+L149R+V258P+A276Q+A364G;

V45L+L149R+V258P+A276S+A364G;

A25W+V45L+L149R+W250Y+A276Q+A364G;

A25W+V45L+L149R+A276Q+L281V+A364G;

A25W+V45L+L149R+A276Q+A286E+A364G;

A25W+V45L+L149R+A276Q+A364G+S388K;

V45L+L149R+V258P+L281V+A364G;

A25W+V45L+L149R+A276Q+A364G;

V45L+L149R+V258P+A364G;

V45L+L149R+V258P+S282N+A364G;

V45L+L149R+A276Q+A364G;

V45L+L149R+A276S+A364G;

V45L+L149R+V258R+A364G; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by n-DSF of at least 5.0° C. compared to the xylanase of SEQ ID NO: 1.

In one aspect, the present invention relates to xylanase variants comprises at least one of the following substitutions or combinations of substitutions:

V45L+A79C+A117S+L149R+S214Q+V258P+A276S+A364G+S388K+D470T+R483V;

V45L+A79C+A117S+L149R+S214Q+V258P+A276S+H358D+A364G+S388K+D470T;

V45L+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+V258P+A276S+A364G+Q384A+S388K+D470T+R483V;

V45L+A79C+A117S+L149R+S214Q+V258P+A276S+H358D+A364G+S388K;

V45L+A117S+L149R+S214Q+V258P+A276S+A364G+S388K+D470T+R483V;

V45L+A117S+L149R+S214Q+V258P+A276S+H358D+A364G+S388K+D470T;

V45L+A79C+A117S+L149R+S214Q+V258P+A276S+A364G+S388K+D470T;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

V45L+A117S+L149R+S214Q+V258P+A276S+H358D+A364G+S388K+D470A;

V45L+A117S+L149R+S214Q+V258P+A276S+H358D+A364G+S388K+L478N;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

D11E+V45L+A117S+L149R+S214Q+V258P+A276S+H358D+A364G+S388K;

V45L+L149R+S214Q+V258P+A276S+A364G+S388K;

V45L+L149R+V258P+A276S+H358T+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

V45L+L149R+S214Q+V258P+A276S+H358T+A364G; and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by n-DSF of at least 10.0° C. compared to the xylanase of SEQ ID NO: 1.

In one aspect, the present invention relates to xylanase variants comprises the following combinations of substitutions: V45L+A79C+A117S+L149R+S214Q+V258P+A276S+H358D+A364G+S388K+D470T and wherein the variant has xylanse activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1, and wherein the variants have increased thermostability measured as increased melting temperature measured by n-DSF of at least 15.0° C. compared to the xylanase of SEQ ID NO: 1.

Parent Xylanase

The parent xylanase may be a polypeptide wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity with SEQ ID No: 1.

In one embodiment, the parent has a sequence identity to the polypeptide of SEQ ID NO: 1 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100%, which have xylanase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 1.

The parent preferably comprises or consists of the amino acid sequence of SEQ ID NO: 1. In one embodiment the parent comprises or consists of the polypeptide of SEQ ID NO: 1. In another embodiment, the parent is an allelic variant of the polypeptide of SEQ ID NO: 1.

In another aspect, the parent is encoded by a polynucleotide that hybridizes under very low stringency conditions, low stringency conditions, medium stringency conditions, medium-high stringency conditions, high stringency conditions, or very high stringency conditions with (i) the mature polypeptide coding sequence, or (ii) the full-length complement of (i) or (ii) (Sambrook et al., 1989, Molecular Cloning, A Laboratory Manual, 2d edition, Cold Spring Harbor, N.Y.).

The polynucleotide or a subsequence thereof, as well as the polypeptide of SEQ ID NO: 1 or a fragment thereof, may be used to design nucleic acid probes to identify and clone DNA encoding a parent from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a parent. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with parent or a subsequence thereof, the carrier material is used in a Southern blot.

The polypeptide may be a hybrid polypeptide in which a region of one polypeptide is fused at the N-terminus or the C-terminus of a region of another polypeptide.

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989, supra).

Preparation of Variants

The xylanase variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

Polynucleotides

The present invention also relates to isolated polynucleotides that encode any of the variants of the present invention. Accordingly, the present invention relates to isolated polynucleotides encoding a variant comprising a substitution at one or more positions corresponding to positions: 45, 149, 364, 258, 276, and 388 of SEQ ID NO: 1 and further comprises a substitution at one or more positions corresponding to positions: 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1, and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity with the amino acid sequence set forth in SEQ ID NO: 1.

The techniques used to isolate or clone a polynucleotide are known in the art and include isolation from genomic DNA or cDNA, or a combination thereof. The cloning of the polynucleotides from genomic DNA can be effected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of 2721i Trichoderma, Lecanicilfium, Simplicillium, Aspergillus, Cornyascus, Acrophialophora, Rhinocladiella, Nemania, Talaromyces, Collariella, Rigidoporous, and/or Loramyces, or a related organism and thus, for example, may be a species variant of the polypeptide encoding region of the polynucleotide.

Modification of a polynucleotide encoding xylanase variant(s) of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

Nucleic Acid Constructs

The present invention also relates to nucleic acid constructs comprising a polynucleotide encoding a variant of the present invention operably linked to one or more (several) control sequences that direct the expression of the coding sequence in a suitable host cell under conditions compatible with the control sequences. Accordingly, the present invention relates to nucleic acid constructs comprising a polynucleotide encoding a variant comprising a substitution at one or more positions corresponding to positions: 45, 149, 364, 258, 276, and 388 of SEQ ID NO: 1 and further comprises a substitution at one or more positions corresponding to positions: 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1, and wherein the polynucleotide is operably linked to one or more control sequences that direct the expression of the coding sequence in a suitable host cell under conditions compatible with the control sequences. The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the polynucleotide of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiol. Rev. 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. Accordingly, the present invention relates to recombinant vectors comprising a polynucleotide encoding a variant comprising a substitution at one or more positions corresponding to positions: 45, 149, 364, 258, 276, and 388 of SEQ ID NO: 1 and further comprises a substitution at one or more positions corresponding to positions: 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1, a promoter, and transcriptional and translational stop signals. The various nucleotide and control sequences may be joined together to produce a recombinant expression vector that may include one or more (several) convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAM111 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more (several) control sequences that direct the production of a variant of the present invention. Accordingly, the present invention relates to recombinant host cells, comprising a polynucleotide encoding a variant comprising a substitution at one or more positions corresponding to positions: 45, 149, 364, 258, 276, and 388 of SEQ ID NO: 1 and further comprises a substitution at one or more positions corresponding to positions: 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1, A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

In some embodiments, at least one of the one or more control sequences is heterologous to the polynucleotide encoding the xylanase variant(s).

In some embodiments, the recombinant host cell comprises at least two copies, e.g., three, four, or five, of the polynucleotide of the present invention.

The host cell may be any microbial or plant cell useful in the recombinant production of a polypeptide of the present invention, e.g., a prokaryotic cell or a fungal cell.

The host cell may be any microbial or plant cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant, comprising: (a) cultivating a host cell of the present invention under conditions suitable for the expression of the variant; and (b) recovering the variant. Accordingly, the present invention relates to methods of producing a variant, comprising (a) cultivating a host cell comprising an expression vector or a polynucleotide encoding a variant comprising a substitution at one or more positions corresponding to positions: 45, 149, 364, 258, 276, and 388 of SEQ ID NO: 1 and further comprises a substitution at one or more positions corresponding to positions: 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1, under conditions suitable for the expression of the variant; and (b) recovering the variant.

In one aspect, the present invention relates to a method of obtaining a xylanase variant, comprising introducing into a parent xylanase a substitution at one or more positions corresponding to positions selected from the group consisting of 45, 149, 364, 258, 276, and 388 of SEQ ID NO: 1 and further comprises a substitution at one or more positions corresponding to positions: 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1, and the variant has xylanase activity; and recovering the variant.

The present invention also relates to methods of producing a variant of the present invention, comprising (a) cultivating a recombinant host cell of the present invention under conditions conducive for production of the variant; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid-state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates. The variant may be detected using methods known in the art that are specific for the variants. These detection methods may include use of specific antibodies, formation of an enzyme product, or disappearance of an enzyme substrate. For example, an enzyme assay may be used to determine the activity of the variant.

The variant may be recovered by methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989) to obtain substantially pure variants.

In an alternative aspect, the variant is not recovered, but rather a host cell of the present invention expressing a variant is used as a source of the variant.

### Fermentation Broth Formulations or Cell Compositions

The present invention also relates to a fermentation broth formulation or a cell composition comprising a xylanase variant of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The present invention also relates to compositions comprising a xylanase variant of the present invention. Preferably, the compositions are enriched in the xylanase variant of the invention. The term “enriched” indicates that the xylanase activity of the composition has been increased, e.g., with an enrichment factor of at least 1.1, such as at least 1.2, at least 1.3, at least 1.4, at least 1.5, at least 2.0, at least 3.0, at least 4.0, at least 5.0, at least 10.

Alternatively, the compositions may comprise multiple enzymatic activities, such as one or more (e.g., several) enzymes selected from the group consisting of xylanse, cellobiohydrolase, cellulase, endoglucanase, and/or arabinofuranosidase.

The compositions may be prepared in accordance with methods known in the art and may be in the form of a liquid or a dry composition. The compositions may be stabilized in accordance with methods known in the art.

Uses

An aspect of the present invention relates to the use of xylanase variant(s) in a method to increase the starch yield and/or gluten yield from corn kernels in a wet milling process.

An aspect of the present invention relates to the use of a xylanase variant of the present invention for producing a fermentation product, such as ethanol, from a gelatinized starch-containing material, using a fermenting organism, such as yeast (e.g., a strain of Saccharomyces, such as Saccharomyces cerevisiae).

An aspect of the present invention relates to the use of a xylanase variant of the present invention for producing a fermentation product, such as ethanol, from an ungelatinized starch-containing material, using a fermenting organism, such as yeast (e.g., a strain of Saccharomyces, such as Saccharomyces cerevisiae).

Another aspect of the present invention relates to the use of a xylanase variant of the present invention for producing a fermentation product, such as ethanol, from a cellulosic-containing material, using a fermenting organism, such as yeast (e.g., a strain of Saccharomyces, such as Saccharomyces cerevisiae).

Another aspect of the present invention relates to the use of a xylanase variant of the present invention for liquefying a starch-containing material.

Another aspect of the present invention relates to the use of a xylanase variant of the present invention for saccharifying a starch-containing material.

Another aspect of the present invention relates to the use of a xylanase variant of the present invention for saccharifying a cellulosic-containing material.

### Processes of the Invention

Processes for Producing Fermentation Products from Un-Gelatinized Starch-Containing Material.

The invention relates to processes for producing fermentation products from starch-containing material without gelatinization (i.e., without cooking) of the starch-containing material (often referred to as a “raw starch hydrolysis” process). The fermentation product, such as ethanol, can be produced without liquefying the aqueous slurry containing the starch-containing material and water. In one embodiment a process of the invention includes saccharifying (e.g., milled) starch-containing material, e.g., granular starch, below the initial gelatinization temperature, preferably in the presence of an alpha-amylase and carbohydrate-source generating enzyme(s) to produce sugars that can be fermented into the fermentation product by a suitable fermenting organism. In this embodiment the desired fermentation product, e.g., ethanol, is produced from un-gelatinized (i.e., uncooked), preferably milled, cereal grains, such as corn.

Processes for producing a fermentation product from starch-containing material may comprise simultaneously saccharifying and fermenting starch-containing material using a carbohydrate-source generating enzymes and a fermenting organism at a temperature below the initial gelatinization temperature of said starch-containing material in the presence of an alpha-amylase of the invention. Saccharification and fermentation may also be separate.

In an aspect, the invention relates to processes for producing fermentation products, preferably ethanol, from starch-containing material comprising the steps of:

i) saccharifying the starch-containing material using a carbohydrate-source generating enzyme at a temperature below the initial gelatination temperature; and

ii) fermenting using a fermenting organism;

wherein at least one or more xylanase variant(s) of the invention are present or added during saccharification, fermentation or simultaneous saccharification and fermentation.

In some embodiments, at least two, at least three, at least four, or at least five xylanase variant(s) of the invention are present and/or added during saccharification, fermentation or simultaneous saccharification and fermentation.

The xylanase variant(s) of the invention are present or added in the above described processes for producing fermentation products from starch-containing material may be added exogenously during saccharification, fermentation or simultaneous saccharification and fermentation as mono-components, as enzyme blends or compositions comprising the xylanase variant(s) of the invention, and/or via in-situ expression and secretion of the xylanase variant(s) of the invention by the fermenting organism, e.g., a recombinant host cell or fermenting organism described herein (e.g., yeast, such as from the genus Saccharomyces, preferably Saccharomyces cerevisiae).

Processes for Producing Fermentation Products from Gelatinized Starch-Containing Material.

In this aspect, the invention relates to processes for producing fermentation products, especially ethanol, from starch-containing material, which process includes a liquefaction step and sequentially or simultaneously performed saccharification and fermentation steps.

Consequently, the invention relates to processes for producing fermentation products from starch-containing material comprising the steps of:

i) liquefying the starch-containing material at a temperature above the initial gelatinization temperature using an alpha-amylase;

ii) saccharifying using a glucoamylase; and

iii) fermenting using a fermenting organism;

wherein at least one or more xylanase variant(s) of the invention are present or added during saccharification, fermentation or simultaneous saccharification and fermentation.

In some embodiments, at least two, at least three, at least four, or at least five xylanase variant(s) of the invention are present and/or added during saccharification, fermentation or simultaneous saccharification and fermentation.

The xylanase variant(s) of the invention are present or added in the above described processes for producing fermentation products from starch-containing material may be added exogenously during saccharification, fermentation or simultaneous saccharification and fermentation as mono-components, as enzyme blends or compositions comprising the xylanase variant(s) of the invention, and/or via in-situ expression and secretion of the xylanase variant(s) of the invention by the fermenting organism, e.g., a recombinant host cell or fermenting organism described herein (e.g., yeast, such as from the genus Saccharomyces, preferably Saccharomyces cerevisiae).

Processes for Producing Fermentation Products from Cellulosic-Containing Material

In this aspect, the invention relates to processes for producing fermentation products, especially ethanol, from cellulosic-containing material, which process may include a pretreatment step and sequentially or simultaneously performed saccharification and fermentation steps.

Consequently, the invention relates to processes for producing fermentation products from cellulosic-containing material comprising the steps of:

i) optionally pretreating a cellulosic-containing material;

ii) saccharifying a cellulosic-containing material and/or pretreated cellulosic-containing material using a carbohydrate-source generating enzyme; and

iii) fermenting using a fermenting organism;

wherein at least one or more xylanase variant(s) of the invention are present or added during saccharifying step ii) or fermenting step iii).

In some embodiments, at least two, at least three, at least four, or at least five xylanase variant(s) of the invention are present and/or added during saccharifying step ii) or fermenting step iii).

The xylanase variant(s) of the invention are present or added in the above described processes for producing fermentation products from cellulosic-containing material may be added exogenously during saccharification, fermentation or simultaneous saccharification and fermentation as mono-components, as enzyme blends or compositions comprising the xylanase variant(s), and/or via in-situ expression and secretion of the xylanase variant(s) by the fermenting organism, e.g., a recombinant host cell or fermenting organism described herein (e.g., yeast, such as from the genus Saccharomyces, preferably Saccharomyces cerevisiae).

Steps ii) and iii) are carried out either sequentially or simultaneously. In a preferred embodiment steps ii) and iii) are carried out simultaneously. The alpha-amylase, an optional thermostable protease, may be added before and/or during liquefaction step i).

A composition of the invention may suitably be used in a process of the invention. A recombinant host cell or fermenting organism of the invention may suitably be used in a process of the invention. However, the enzymes may also be added separately.

Whether the process of the invention includes or does not include a liquefaction step or pretreatment step, the essential feature of the invention is that at xylanase variant(s) of the invention are present or added during liquefaction, saccharification, fermentation or simultaneous saccharification and fermentation. In one embodiment, at least one xylanase variant of the present invention is present or added during liquefaction. In one embodiment, at least one xylanase variant of the present invention is present or added during fermentation or simultaneous saccharification and fermentation. As noted above, the xylanase variant(s) may be added exogenously as a standalone enzyme or an enzyme blend or composition comprising at least one, at least two, at least three, at least four, or at least five xylanase variant(s), or expressed and secreted in situ by a recombinant host cell or fermenting organism of the present invention comprising at least one, at least two, at least three, at least four, or at least five xylanase variant(s).

Examples of other enzymes that can be added in addition to one or more xylanase variant(s) of the present invention during saccharification/fermentation/SSF, or used as a component of an enzyme blend or composition of the invention include, without limitation, alpha-amylases, endoglucanase, cellobhiohydrolases, beta-glucosidases, glucoamylases, hemicellulases, cellulases. Particularly, saccharification and/or fermentation or simultaneous saccharification and fermentation, is performed in the presence of at least one cellulase/cellulolytic composition. More particularly the cellulases/cellulolytic composition are derived from a strain of Trichoderma, in particular Trichoderma reesei, or a strain of Humicola, in particular Humicola insolens, or a strain of Chrysosporium, in particular Chrysosporium lucknowense. The cellulases/cellulolytic composition should at least comprise a beta-glucosidase, a cellobiohydrolase and an endoglucanase.

The pH during liquefaction may be between 4-7. In an embodiment, the pH during liquefaction is from 4.5-5.0, such as between 4.5-4.8. In another embodiment liquefaction is carried out at a pH above 5.0-6.5, such as above 5.0-6.0, such as above 5.0-5.5, such as between 5.2-6.2, such as around 5.2, such as around 5.4, such as around 5.6, such as around 5.8.

According to the invention the temperature is above the initial gelatinization temperature. The term “initial gelatinization temperature” refers to the lowest temperature at which solubilization of starch, typically by heating, begins. The temperature can vary for different starches.

In an embodiment, the temperature during liquefaction step i) is in the range from 70-100° C., such as between 75-100° C., preferably between 80-100° C., such as between 85-95° C., such as around between 88 and 92° C.

The aqueous slurry may contain from 10-55 w/w-% dry solids (DS), preferably 25-45 w/w-% dry solids (DS), more preferably 30-40 w/w-% dry solids (DS) of starch-containing material.

Liquefaction step i) is according to the invention carried out for 0.5-5 hours, such as 1-3 hours, such as typically around 2 hours.

The aqueous slurry may in an embodiment be jet-cooked to further gelatinize the slurry before being subjected to liquefaction in step i). The jet-cooking may be carried out at a temperature between 110-145° C., preferably 120-140° C., such as 125-135° C., preferably around 130° C. for about 1-15 minutes, preferably for about 3-10 minutes, especially around about 5 minutes.

Saccharification and Fermentation

One or more carbohydrate-source generating enzymes, in particular glucoamylase, may be present and/or added during saccharification step ii) and/or fermentation step iii). The carbohydrate-source generating enzyme may preferably be a glucoamylase, but may also be an enzyme selected from the group consisting of: beta-amylase, maltogenic amylase and alpha-glucosidase. The carbohydrate-source generating enzyme added during saccharification step ii) and/or fermentation step iii) is typically different from the optional carbohydrate-source generating enzyme, in particular thermostable glucoamylase, optionally added during liquefaction step i). In a preferred embodiment the carbohydrate-source generating enzymes, in particular glucoamylase, is added together with a fungal alpha-amylase.

When doing sequential saccharification and fermentation, saccharification step ii) may be carried out at conditions well-known in the art. For instance, the saccharification step ii) may last up to from about 24 to about 72 hours. In an embodiment, pre-saccharification is done. Pre-saccharification is typically done for 40-90 minutes at a temperature between 30-65° C., typically about 60° C. Pre-saccharification is in an embodiment followed by saccharification during fermentation in simultaneous saccharification and fermentation (“SSF). Saccharification is typically carried out at temperatures from 20-75° C., preferably from 40-70° C., typically around 60° C., and at a pH between 4 and 5, normally at about pH 4.5.

Simultaneous saccharification and fermentation (“SSF”) is widely used in industrial scale fermentation product production processes, especially ethanol production processes. When doing SSF the saccharification step ii) and the fermentation step iii) are carried out simultaneously. There is no holding stage for the saccharification, meaning that a fermenting organism, such as yeast, and enzyme(s), may be added together. However, it is also contemplated to add the fermenting organism and enzyme(s) separately. SSF is according to the invention typically carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours. In an embodiment the pH is between 3.5-5, in particular between 3.8 and 4.3.

Fermenting Organisms

The term “Fermenting organism” refers to any organism, including bacterial and fungal organisms, especially yeast, suitable for use in a fermentation process and capable of producing the desired fermentation product. Especially suitable fermenting organisms are able to ferment, i.e., convert, sugars, such as glucose or maltose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

Examples of commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties). Other useful yeast strains are available from biological depositories such as the American Type Culture Collection (ATCC) or the Deutsche Sammlung von Mikroorganismen and Zellkulturen GmbH (DSMZ), such as, e.g., BY4741 (e.g., ATCC 201388); Y108-1 (ATCC PTA.10567) and NRRL YB-1952 (ARS Culture Collection). Still other S. cerevisiae strains suitable as host cells DBY746, [Alpha][Eta]22, 5150-2B, GPY55-15Ba, CEN.PK, USM21, TMB3500, TMB3400, VTT-A-63015, VTT-A-85068, VTT-c-79093 and their derivatives as well as Saccharomyces sp. 1400, 424A (LNH-ST), 259A (LNH-ST) and derivatives thereof.

The fermenting organisms may be a host cell that expresses a heterologous xylanase variant(s) of the invention (e.g., any xylanase variant(s) described herein). Any xylanase variant(s) contemplated for a process, method, enzyme blend, or composition described herein is also contemplated for expression by a fermenting organism or host cell.

In one embodiment is a recombinant host cell (e.g., yeast host cell, such as a strain of Saccharomyces, for example Saccharomyces cerevisiae) comprising a heterologous polynucleotide encoding xylanase variant(s) (e.g., xylanase) (e.g., any xylanase described herein).

In one embodiment, a recombinant yeast host cell comprising a heterologous polynucleotide encoding a xylanase variant(s), wherein the polynucleotide comprises, consists, or consists essentially of the nucleotide sequence, or is a polynucleotide comprising, consisting of, or consisting essentially of a nucleotide sequence having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to the polypeptide of SEQ ID NO: 1, which has xylanase activity.

The fermenting organisms may be a host cell that expresses heterologous polynucleotides encoding enzymes other than the xylanase of the present invention, or that expresses such enzymes in addition to the xylanase of the present invention.

Starch-Containing Material

Any suitable starch-containing material containing may be used according to the present invention. The starting material is generally selected based on the desired fermentation product. Examples of starch-containing materials, suitable for use in a process of the invention, include whole grains, corn, wheat, barley, rye, milo, sago, cassava, tapioca, sorghum, rice, peas, beans, or sweet potatoes, or mixtures thereof or starches derived there from, or cereals. Contemplated are also waxy and non-waxy types of corn and barley. In a preferred embodiment, the starch-containing material in a process for producing a fermentation product, wherein the fermentation product is ethanol, is corn or wheat.

Fermentation Products

The term “fermentation product” means a product produced by a process including a fermentation step using a fermenting organism. A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an (e.g., pentane, hexane, heptane, octane, nonane, decane, undecane, and dodecane), a cycloalkane (e.g., cyclopentane, cyclohexane, cycloheptane, and cyclooctane), an alkene (e.g., pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide.

In one embodiment, the fermentation product is an alcohol. The term “alcohol” encompasses a substance that contains one or more hydroxyl moieties. The alcohol can be, but is not limited to, n-butanol, isobutanol, ethanol, methanol, arabinitol, butanediol, ethylene glycol, glycerin, glycerol, 1,3-propanediol, sorbitol, xylitol. See, for example, Gong et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30(2): 117-124; Ezeji et al., 2003, World Journal of Microbiology and Biotechnology 19(6): 595-603. In one embodiment, the fermentation product is ethanol.

Preferably processes of the invention are used for producing an alcohol, such as ethanol. The fermentation product, such as ethanol, obtained according to the invention, may be used as fuel, which is typically blended with gasoline. However, in the case of ethanol it may also be used as potable ethanol.

Recovery

The fermentation product, e.g., ethanol, can optionally be recovered from the fermentation medium using any method known in the art including, but not limited to, chromatography, electrophoretic procedures, differential solubility, distillation, or extraction. For example, alcohol is separated from the fermented cellulosic material or fermented starch-containing material and purified by conventional methods of distillation. As another example, the desired fermentation product may be extracted from the fermentation medium by micro or membrane filtration techniques. Ethanol with a purity of up to about 96 vol. % can be obtained, which can be used as, for example, fuel ethanol, drinking ethanol, i.e., potable neutral spirits, or industrial ethanol.

### Wet Milling Process

Corn kernels are wet milled in order to open up the kernels and separate the kernels into its four main constituents: starch, germ, fiber and gluten.

The wet milling process can vary significantly from mill to mill, however conventional wet milling usually comprises the following steps:

1. Steeping

2. Grinding

3. Separation into streams comprising:

i) germ; and ii) fiber, starch and gluten

4. Fine grinding

5. Fiber washing, pressing and drying

6. Starch/gluten separation, and

7. Starch washing.

### Steeping, Grinding and Germ Separation

Corn kernels are softened by soaking in water for between about 30 minutes to about 48 hours, preferably 30 minutes to about 15 hours, such as about 1 hour to about 6 hours at a temperature of about 50° C., such as between about 45° C. to 60° C. During steeping, the kernels absorb water, increasing their moisture levels from 15 percent to 45 percent and more than doubling in size. The optional addition of e.g. 0.1 percent sulphur dioxide (SO₂) and/or NaHSO₃ to the water prevents excessive bacteria growth in the warm environment. As the corn swells and softens, the mild acidity of the steep water begins to loosen the gluten bonds within the corn and release the starch. After the corn kernels are steeped they are cracked open to release the germ usually by a course grinding step. The germ contains corn oil. The germ is separated from the heavier density mixture of starch, gluten and fiber essentially by “floating” the germ segment free of the other substances under closely controlled conditions. This method serves to eliminate any adverse effect of traces of corn oil in later processing steps. Subsequently the germ may be dried and oil extracted. After separating the germ, the ground kernel mass comprising fiber, starch and gluten (protein) is usually subjected to a fine grinding step.

### Fiber Washing, Pressing and Drying

To get maximum starch and gluten recovery, while keeping any fiber in the final product to an absolute minimum, it is necessary to wash the free starch and gluten from the fiber during processing. Therefore, the finely ground kernel mass is subjected to a fiberwashing procedure. Thereby free starch and gluten is separated from fiber during screening (washing) and collected as mill starch. The remaining fiber is then pressed to decrease the water content.

To get maximum starch and gluten recovery, while keeping any fiber in the final product to an absolute minimum, it is necessary to wash the free starch and gluten from the fiber fraction during processing. The fiber is collected, slurried and screened, typically after soaking, grinding and separation of germs from the corn kernels, to reclaim any residual starch or gluten in the corn kernel mass. This process is herein referred to as the fiber washing procedure/step.

### Starch Gluten Separation

The starch-gluten suspension from the fiber-washing step, called mill starch, is separated into starch and gluten. Gluten has a low density compared to starch. By passing mill starch through a centrifuge, the gluten is readily spun out.

### Starch Washing

The starch slurry from the starch separation step contains some insoluble protein and much of solubles. They have to be removed before a top quality starch (high purity starch) can be made. The starch, with just one or two percent protein remaining, is diluted, washed 8 to 14 times, re-diluted and washed again in hydro-clones to remove the last trace of protein and produce high quality starch, typically more than 99.5% pure.

Products of wet milling: Wet milling can be used to produce, without limitation, corn steep liquor, corn gluten feed, germ, corn oil, corn gluten meal, corn starch, modified corn starch, syrups such as corn syrup, and corn ethanol.

An aspect of the present invnetion is to provide a method to increase the total starch yield and/or gluten yield that can be obtained from corn kernels in a wet milling process, the method comprising: contacting corn kernels or a fraction of the corn kernels, particularly a fiber rich fraction, with an effective amount of a xylanase variant of the invention and optionally one or more hydrolytic enzymes.

In another specific embodiment the present invention provides a method to increase the total starch yield and/or gluten yield that can be obtained from corn kernels in a wet milling process, the method comprising the steps of:

a) soaking the corn kernels in water to produce soaked kernels;

b) grinding the soaked kernels to produce ground kernels;

c) separating germ from the ground kernels to produce a corn kernel mass comprising fiber, starch and gluten; and

d) subjecting the corn kernel mass to a fiber washing procedure separating starch and gluten from the fiber;

wherein at least a xylanase variant of the invention is present/added before or during step d).

The method may further comprise the steps of:

e) separating the starch from the gluten; and optionally

f) washing the starch.

Optionally the corn kernel mass comprising fiber, starch and gluten from step c) is subjected to a further grinding step, preferably a fine grinding step before the fiber washing procedure in step d).

In one embodiment, said corn kernels or a fiber rich fraction of said corn kernels is admixed with optionally with said one or more hydrolytic enzymes, before, or preferably during the step of subjecting the corn kernel mass to a fiber washing procedure.

In one embodiment, hydrolytic enzymes suitable for use in the method of the invention comprise one or more enzymes selected form the group consisting of: cellulases (EC 3.2.1.4), xylanases (EC 3.2.1.8), arabinofuranosidases (EC 3.2.1.55 (Non-reducing end alpha-L-arabinofuranosidases); EC 3.2.1.185 (Non-reducing end beta-L-arabinofuranosidases), cellobiohydrolase I (EC 3.2.1.150), cellobiohydrolase II (E.C. 3.2.1.91), cellobiosidase (E.C. 3.2.1.176), beta-glucosidase (E.C. 3.2.1.21), beta-xylosidases (EC 3.2.1.37), and proteases (E.C 3.4).

Preferably the enzymes are selected from cellulases.

In one embodiment the hydrolytic enzymes comprise one or more cellulases. The cellulases may be selected from at least the group consisting of an endoglucanase (EG), and a cellobiohydrolase (CBH). More particularly, the cellulase(s) comprisies one or more enzyme selected from the group consisting of an endoglucanase, a cellobiohydrolase I, a cellobiohydrolase II, or a combination thereof.

In one embodiment the hydrolytic enzymes further comprise an arabinofuranosidase. The arabinofuranosidase may be selected from the group consisting of a GH43 polypeptide, a GH62 polypeptide, GH51 polypeptide. Particularly a GH62 polypeptide.

In one embodiment, the one or more hydrolytic enzymes is expressed in an organism with a cellulase background, such as Trichoderma reesei. According to these embodiments the xylanase and or arabinofuranosidase polypeptides defined according to the invention is/are expressed together with endogenous cellulases from Trichoderma.

In one embodiment, the enzyme composition comprising one or more hydrolytic enzymes may comprise cellulases derived from Trichoderma reesei or Humicula insolens. In one particular embodiment the celullases are derived from Trichoderma reesei background cellulases and having a CBH I and CBH II derived from Aspergillus fumigatus.

In one embodiment, the one or more hydrolytic enzymes are purified. The purified enzymes may be used in an enzyme composition as described in other embodiments of the present invention.

In one embodiment, the one or more hydrolytic enzymes is/are in a liquid composition. The composition may be homogenous or heterogeneous.

In one embodiment, the one or more hydrolytic enzymes is/are in a solid composition.

In one embodiment, the effective amount of one or more hydrolytic enzymes admixed with one or more fractions of said corn kernel mass, is between 0.005-0.5 kg enzyme protein (EP)/metric tonne (MT) corn kernels entering the wet milling process, such as between 0.010-0.5 kg EP/MT corn kernel, such as between 0.05-0.5 kg/MT corn kernel or 0.075-0.5 kg/MT or 0.1-0.5 kg/MT corn kernel or 0.005-0.4 kg/MT corn kernel or 0.01-0.4 kg/MT corn kernel or 0.05-0.4 kg/MT corn kernel or 0.075-0.4 kg/MT corn kernel or 0.1-0.4 kg/MT corn kernel or 0.005-0.3 kg/MT corn kernel or 0.01-0.3 kg/MT corn kernel or 0.05-0.3 kg/MT corn kernel or 0.075-0.3 kg/MT or 0.1-0.3 kg/MT corn kernel or 0.005-0.2 kg/MT corn kernel or 0.010-0.2 kg/MT corn kernel or 0.05-0.2 kg/MT corn kernel or 0.075-0.2 kg/MT or 0.1-0.2 kg/MT corn kernel or such as 0.075-0.10 kg/MT corn kernel or 0.075-0.11 kg/MT corn kernel.

In preferred embodiments the enzyme composition comprises cellulase obtained from a culture of Trichoderma reesei, such as a culture of Trichoderma reesei ATCC 26921. Suitable cellulases are available; e.g. from Novozymes A/S under the commercial name Celluclast®.

In a preferred embodiment, said corn kernels or a fraction of said corn kernels is admixed with an enzyme composition comprising comprising a xylanase variant of the present invention and one or more hydrolytic enzymes during the step of subjecting the corn kernel mass to a fiber washing procedure.

In one embodiment, said corn kernels or a fraction of said corn kernels is allowed to react with said an enzyme composition comprising a xylanase variant and one or more hydrolytic enzymes for at least 15 minutes, such as at least 20 minutes, at least 25 minutes, at least 30 minutes, at least 35 minutes, at least 40 minutes, at least 45 minutes, at least 50 minutes, at least 55 minutes, at least 60 minutes, at least 70 minutes, at least 80 minutes, at least 90 minutes, at least 100 minutes, at least 110 minutes or at least 120 minutes.

The specific equipment used in the fiber washing procedure can vary, but the main principle of the process remains the same.

In one embodiment, the incubation temperature in said fiber washing system is between 25 and 95° C., such as between 25 and 90° C., 25 and 85° C., 25 and 80° C., 25 and 75° C., 25 and 70° C., 25 and 65° C., 25 and 60° C., 25 and 55° C., 25 and 53° C., 25 and 52° C., 30 and 90° C., 30 and 85° C., 30 and 80° C., 30 and 75° C., 30 and 70° C., 30 and 65° C., 30 and 60° C., 30 and 55° C., 30 and 53° C., 30 and 52° C., 35 and 90° C., 35 and 85° C., 35 and 80° C., 35 and 75° C., 35 and 70° C., 35 and 65° C., 35 and 60° C., 35 and 55° C., 35 and 53° C., 35 and 52° C., 39 and 90° C., 39 and 85° C., 39 and 80° C., 39 and 75° C., 39 and 70° C., 39 and 65° C., 39 and 60° C., 39 and 55° C., 39 and 53° C., 39 and 52° C.

In one embodiment, the enzyme composition comprising one or more hydrolytic enzymes may comprise cellulases expressed in Trichoderma reesei and other hydrolotic enzymes which are added to the enzyme composition in a purified or semi-purified form.

In one embodiment, the one or more hydrolytic enzymes are purified. The purified enzymes may be used in an enzyme composition as described in other embodiments of the present invention.

In one embodiment, the one or more hydrolytic enzymes is/are in a liquid composition. The composition may be homogenous or heterogeneous.

In one embodiment, the one or more hydrolytic enzymes is/are in a solid composition.

The invention is further defined in the following paragraphs:

Paragraph 1. A xylanase variant of a parent xylanase comprising a substitution at one or more positions corresponding to position 45, 149, 364, 258, 276, and 388 of SEQ ID NO: 1 and wherein said variant has xylanase activity and wherein said variant has at least 60%, at least 65%, at least 70%, at least 75%, at least 80%, at least 85%, at least 90%, at least 95%, at least 96%, at least 97%, at least 98%, or at least 99%, but less than 100% sequence identity sequence identity to the polypeptide of SEQ ID NO: 1.

Paragraph 2. The xylanase variant according to paragraph 1, which has increased thermostability compared to the xylanase of SEQ ID NO: 1.

Paragraph 3. The xylanase variant according to paragraphs 1-2, wherein said increased thermostability is measured as increased melting temperature using TSA of at least 0.5° C., at least 1° C., at least 1.5° C., at least 2° C., at least 2.5° C., at least 3° C., at least 3.5° C., at least 4.0° C., at least 4.5° C. or at least 5° C.

Paragraph 4. The xylanase variant according to paragraphs 1-2, wherein said increased thermostability is measured as increased melting temperature using n-DSF of at least 0.5° C., at least 1° C., at least 1.5° C., at least 2° C., at least 2.5° C., at least 3° C., at least 3.5° C., at least 4.0° C., at least 4.5° C. or at least 5° C.

Paragraph 5. The xylanase variant according to any one of the preceding paragraphs further comprising a substitution at one or more positions corresponding to position to 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1.

Paragraph 6. The xylanase variant according to paragraph 5, wherein said variant comprises one or more of the following substitutions at a position corresponding to positions D9E, D11E, A18V, 519T, A20H, A20P, A24H, A24K, A25W, M28P, C40A, C40L, C40S, C40V, C60A, C60L, C60S, C60V, Q61K, Q61R, C69A, C69L, C69S, C69V, T70R, Q77E, A79C, S92D, S92N, A93S, A93T, D95N, D113T, A117C, A117S, N127D, C161L, C161S, C161V, N163H, N168G, A170S, S173Y, T180Q, G187K, G187L, G187S, S190E, D192E, T211H, S214E, S214Q, S214Y, T215I, T215K, T215V, H219I, H219V, V234P, C243L, C243S, C243V, W250Y, A252S, N260H, L281V, S282N, A286E, A286S, Q314A, H334D, A340G, V350L, N355G, L356M, H358D, H358T, T366I, T366V, C375A, C375L, C375S, C375V, Q384A, Q385C, Y386G, S389H, S389V Y391V, V393H, V393R, G394A, G394D, G394Q, N395D, C396A, C396L, C396A, C396S, C396V, D446T, R459V, 0468A, C468L, C468S, C468V, I469T, D470A, D470K, D470T, D470V, L471R, A472I, A472L, S473G, S473K, N474G, N476D, N476N, T477K, T477V, L478N, L478P, T480L, T480N, S481A, R483V, L484D, L484K, L484S, W495E, W495I, W495S, Q496E, Q496W, Q496R, V497D, V497E, V497P, V497W, V498I, A499L, A499Y, V500T, S501E, S501N, G503L, C522A, C522S, C522V, D525L, G526T, N527H, S528H, N529T, L530R, C540A, C540L, C540S, and C540V of SEQ ID NO: 1.

Paragraph 7. The variant of any of the preceding paragraphs, wherein the variant comprises at least one of the following substitutions or combinations of substitutions:

V45L;

V45N;

V451;

V45T;

V45C;

L149R;

V258P;

V258R;

A276S;

A276C;

A276Q;

A276F;

A276N;

A276T;

A276Y;

A364G;

A364A;

5388A;

S388H;

5388L;

S388R;

V45L+L149R;

V45N+L149R;

L149R+A364G;

V258P+A276Q;

V258P+A276S;

A25W+L149R;

V45 L+A276S;

A25W+A276S;

V45 L+A276Q;

V45L+V258P;

A25W+V258P;

A25W+A276Q;

L149R+W250Y;

V45 L+S388K;

V45 L+A364G;

V45N+V258P;

V258P+S388K;

A25W+V45 L;

A276Q+S388K;

W250Y+A276Q;

A276S+S388K;

A276Q+L281V;

W250Y+A276S;

A25W+S388K;

A276S+L281V;

G187S+A276Q;

S92 D+A276S;

L149R+V258R;

V45N+S388K;

A25W+A364G;

V258P+L281V;

V258R+A276S;

G187S+A276S;

G187K+A276S;

V45L+S92D;

V45L+L281V;

V45N+W250Y;

V258R+A276Q;

V45L+V258R;

W250Y+S388K;

V45N+G187K;

A25W+V258R;

G187K+S388K;

A25W+S92D;

L281V+S388K;

V45N+V258R;

L281V+A364G;

S92 D+A364G;

A25W+G187S;

A25W+G187K;

W250Y+V258P;

S92 D+V258P;

G187K+W250Y;

V45N+S92D;

G187S+A364G;

G187S+L281V;

G187S+S388K;

G187S+V258R;

S92D+G187S;

V45L+D11E;

L149R+S388K;

L149R+D11E;

A364G+D11E;

S388K+D11E;

V45L+L149R+V258P;

V45L+L149R+A364G;

V45L+L149R+W250Y;

V45L+L149R+G187S;

V45L+L149R+S282N;

V45L+S92D+L149R;

V45L+L149R+A252S+H334D;

V45L+L149R+S190E+D446T;

V45L+L149R+V258P+A364G;

V45L+L149R+A276Q+A364G;

V45L+L149R+A276S+A364G;

V45L+L149R+V258P+A276S;

V45L+L149R+L281V+A364G;

L149R+V258P+A276S+A364G;

V45L+V258P+A276S+A364G;

A25W+V45L+L149R+A364G;

V45L+L149R+V258R+A364G;

V45L+L149R+G187K+A364G;

V45L+L149R+G187S+A364G;

V45L+L149R+W250Y+A364G;

V45L+A93S+L149R+A252S+H334D;

V45L+A93S+L149R+A252S+H334D;

V45L+A93S+L149R+V234P+A252S;

V45L+A93S+L149R+A252S+H334D;

V45L+A93S+L149R+A252S+H334D;

V45L+A93S+L149R+A252S+H334D;

V45L+L149R+S190E+D446T+R459V;

V45L+L149R+V258P+A276S+A364G;

V45L+L149R+V258P+A276Q+A364G;

A25W+V45L+L149R+V258P+A276S;

V45L+L149R+A276Q+L281V+A364G;

V45 L+L149R+A276Q+S282N+A364G;

A25W+V45L+L149R+V258P+A276Q;

V45 L+T70 R+L149R+A276Q+A364G;

V45L+L149R+V258P+L281V+A364G;

A25W+V45L+L149R+A276Q+A364G;

V45L+L149R+A276Q+Q314A+A364G;

V45L+L149R+G187K+V258P+A364G;

V45 L+L149R+A276Q+A286E+A364G;

V45 L+L149R+A276Q+H358T+A364G;

A25W+V45N+L149R+V258P+A276S;

V45L+N127D+L149R+A276Q+A364G;

V45 L+L149R+W250Y+A276Q+A364G;

V45 L+L149R+W250Y+V258P+A364G;

V45L+L149R+A276Q+A364G+S388K;

A25W+V45L+L149R+V258P+A364G;

V45L+L149R+N168G+A276Q+A364G;

V45L+A117S+L149R+A276Q+A364G;

A24K+V45 L+L149R+A276Q+A364G;

V45 L+L149R+A276Q+A286S+A364G;

V45 L+L149R+A170S+A276Q+A364G;

V45L+L149R+T180Q+A276Q+A364G;

V45 L+L149R+A276Q+V350 L+A364G;

V45L+L149R+V258P+S282N+A364G;

A25W+V45L+L149R+A276S+A364G;

V45L+L149R+G187S+V258P+A364G;

A25W+L149R+V258P+A276S+A364G;

V45L+L149R+G187L+A276Q+A364G;

A25W+V45L+V258P+A276S+A364G;

V45L+L149R+N260H+A276Q+A364G;

V45L+L149R+W250Y+V258R+A364G;

V45L+L149R+N163H+A276Q+A364G;

V45L+S92D+L149R+A276Q+A364G;

V45 L+L149R+S173Y+A276Q+A364G;

V45L+S92D+L149R+V258P+A364G;

V45L+D113T+L149R+A276Q+A364G;

V45L+L149R+G187S+V258R+A364G V45L+L149R+V258R+A364G+S388K;

V45L+L149R+G187K+V258R+A364G;

V45L+L149R+V258R+S282N+A364G;

V45L+L149R+V258R+L281V+A364G;

V45L+S92N+L149R+V258P+A364G;

V45L+L149R+V258P+A364G+S388K;

V45L+S92N+L149R+V258R+A364G;

A25W+V45L+A79C+L149R+V258P+A276S;

V45 L+L149R+V258P+A276S+H358T+A364G;

A25W+V45L+L149R+V258P+A276S+A364G;

V45 L+L149R+V258P+A276S+L281V+A364G;

V45L+A93S+L149R+A252S+H334D+A340G;

V45L+L149R+V258P+A276S+A364G+S388K;

V45L+L149R+N168G+V258P+A276S+A364G;

V45L+L149R+S173Y+V258P+A276S+A364G;

V45L+A93S+L149R+A252S+H334D+A340G;

A25W+V45L+L149R+V258P+A276S+H358T;

A25W+V45L+L149R+V258P+A276Q+A364G;

A25W+V45L+L149R+S214E+V258P+A276S;

V45L+L149R+A170S+V258P+A276S+A364G;

A25W+V45L+L149R+S214Q+V258P+A276S;

A25W+V45L+L149R+V258P+A276S+S388K;

V45L+A93S+L149R+V234P+A252S+H334D;

V45L+N127D+L149R+V258P+A276S+A364G;

V45 L+L149R+W250Y+V258P+A276S+A364G;

A25W+V45L+L149R+W250Y+A276Q+A364G;

V45L+L149R+T180Q+V258P+A276S+A364G;

V45 L+L149R+V258P+A276S+Q314A+A364G;

V45L+A117S+L149R+V258P+A276S+A364G;

V45N+L149R+V258P+A276S+A364G+S388K;

V45 L+L149R+V258P+A276Q+L281V+A364G;

A25W+V45L+L149R+V258P+A276S+Q314A;

V45 L+L149R+V258P+A276S+A286E+A364G;

A25W+V45L+L149R+A276Q+L281V+A364G;

A25W+V45L+L149R+V258P+A276S+A286E;

A25W+V45L+L149R+V258P+A276S+L281V;

A25W+V45L+D113T+L149R+V258P+A276S;

A25W+V45L+L149R+T180Q+V258P+A276S;

A25W+V45L+L149R+V258P+A276S+A286S;

V45L+L149R+G187L+A276Q+L281V+A364G;

V45L+L149R+A276Q+L281V+V350L+A364G;

V45L+L149R+A276Q+L281V+A364G+S388K;

A25W+V45L+S92D+L149R+V258P+A276S;

A25W+V45L+L149R+G187L+V258P+A276S;

A25W+V45L+L149R+W250Y+V258P+A276S;

V45L+D113T+L149R+V258P+A276S+A364G;

V45L+T70R+L149R+A276Q+L281V+A364G;

V45L+S92D+L149R+A276Q+L281V+A364G;

V45L+D113T+L149R+A276Q+L281V+A364G;

V45L+L149R+A276Q+L281V+Q314A+A364G;

A25W+V45L+Q61K+L149R+V258P+A276S;

D9E+A25W+V45L+L149R+V258P+A276S;

V45L+T70R+L149R+V258P+A276S+A364G;

V45L+L149R+N163H+V258P+A276S+A364G;

V45L+L149R+T180Q+A276Q+L281V+A364G;

V45 L+L149R+A276Q+L281V+A286S+A364G;

A25W+V45L+L149R+A170S+V258P+A276S;

A25W+V45L+T70R+L149R+V258P+A276S;

V45L+L149R+N168G+A276Q+L281V+A364G;

V45 L+L149R+S173Y+A276Q+L281V+A364G;

V45 L+L149R+W250Y+A276Q+L281V+A364G;

A25W+V45L+L149R+A276Q+A286E+A364G;

A25W+V45L+L149R+A276Q+A364G+S388K;

V45L+N127D+L149R+A276Q+L281V+A364G;

V45 L+L149R+A276Q+L281V+A286E+A364G;

A25W+V45L+A117S+L149R+V258P+A276S;

A24K+V45L+L149R+V258P+A276S+A364G;

V45L+L149R+V258P+A276S+S282N+A364G;

V45L+A117S+L149R+A276Q+L281V+A364G;

V45L+L149R+N260H+A276Q+L281V+A364G;

A25W+V45L+L149R+N168G+V258P+A276S;

A25W+V45L+L149R+G187S+V258P+A276S;

A25W+V45L+L149R+V258P+A276S+V350L;

A25W+V45L+Q61R+L149R+V258P+A276S;

V45N+L149R+V258P+A276S+L281V+A364G;

A25W+V45L+L149R+V258P+A276S+S282N;

A25W+V45L+L149R+S173Y+V258P+A276S;

A25W+V45L+L149R+A276Q+A286S+A364G;

A25W+V45L+L149R+T215V+V258P+A276S;

A25W+V45L+L149R+T215I+V258P+A276S;

A25W+V45L+L149R+T211H+V258P+A276S;

V45 L+L149R+V258P+A276S+V350L+A364G;

A25W+V45L+D113T+L149R+A276Q+A364G;

A25W+V45N+L149R+V258P+A276S+A364G;

A25W+V45L+L149R+H219I+V258P+A276S;

M28P+V45L+L149R+V258P+A276S+A364G;

A25W+V45L+T70 R+L149R+A276Q+A364G;

A25W+V45L+L149R+A276Q+Q314A+A364G;

A25W+V45L+L149R+S214Y+V258P+A276S;

V45L+L149R+A276Q+L281V+H358T+A364G;

A25W+V45L+L149R+H219V+V258P+A276S;

D11E+A25W+V45L+L149R+V258P+A276S;

A25W+V45L+A117S+L149R+A276Q+A364G;

A25W+V45L+L149R+G187L+A276Q+A364G;

V45N+L149R+V258P+A276S+H358T+A364G;

A25W+V45L+L149R+N163H+A276Q+A364G;

A25W+V45L+L149R+S173Y+A276Q+A364G;

A25W+V45L+L149R+A276Q+S282N+A364G;

A25W+V45L+L149R+A276Q+H358T+A364G;

A25W+V45L+L149R+T180Q+A276Q+A364G;

A25W+V45L+N127D+L149R+A276Q+A364G;

A25W+V45L+L149R+A170S+A276Q+A364G;

A25W+V45L+N127D+L149R+V258P+A276S;

A25W+V45L+L149R+N168G+A276Q+A364G;

A25W+V45L+L149R+N260H+A276Q+A364G;

A25W+V45L+L149R+G187K+V258P+A276S;

A25W+M28P+V45L+L149R+V258P+A276S;

V45L+L149R+N163H+A276Q+L281V+A364G;

V45L+L149R+G187L+V258P+A276S+A364G;

A25W+V45 L+L149R+A276Q+V350L+A364G;

V45L+L149R+V258P+A276S+N355G+A364G;

A25W+V45L+L149R+V258P+A276S+N355G;

A25W+V45L+S92D+L149R+A276Q+A364G;

A25W+V45L+L149R+A276Q+N355G+A364G;

V45L+A79C+L149R+V258P+A276S+A364G+S388K;

V45N+A79C+L149R+V258P+A276S+A364G+S388K;

V45L+L149R+S214Q+V258P+A276S+H358D+A364G;

A25W+V45L+A79C+L149R+V258P+A276S+A364G;

V45L+A79C+L149R+V258P+A276S+L281V+A364G;

A25W+V45L+L149R+S214E+V258P+A276S+A364G;

V45L+L149R+S214Q+V258P+A276Q+A364G+S388K;

V45N+L149R+S214Q+V258P+A276S+A364G+S388K;

V45L+L149R+S214Q+V258P+A276S+A364G+S388K;

V45L+L149R+S214E+V258P+A276S+H358T+A364G;

A25W+V45L+L149R+S214E+V258P+A276Q+A364G;

V45L+L149R+S214Q+V258P+A276S+L281V+A364G;

V45L+N127D+L149R+V258P+A276S+A364G+S388K;

V45L+L149R+V258P+A276S+H358T+A364G+S388K;

V45L+L149R+G187L+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

A25W+V45L+L149R+V258P+A276S+A364G+S388K;

V45L+L149R+S214Q+V258P+A276S+H358T+A364G;

A25W+V45L+L149R+S214Q+V258P+A276S+A364G;

V45L+L149R+N168G+V258P+A276S+A364G+S388K;

V45L+A117S+L149R+V258P+A276S+A364G+S388K;

A25W+V45L+L149R+V258P+A276S+H358T+A364G;

V45L+L149R+V258P+N260H+A276S+A364G+S388K;

V45L+L149R+T215I+V258P+A276S+A364G+S388K;

V45L+Q61K+L149R+V258P+A276S+H358T+A364G;

V45L+L149R+V258P+A276S+Q314A+H358T+A364G;

V45L+L149R+T180Q+V258P+A276S+A364G+S388K;

V45L+L149R+V258P+A276S+Q314A+A364G+S388K;

A25W+V45L+L149R+V258P+A276S+A286S+A364G;

V45L+L149R+H219I+V258P+A276S+H358T+A364G;

V45L+L149R+V258P+A276S+V350L+A364G+S388K;

D11E+V45L+L149R+V258P+A276S+A364G+S388K;

V45L+D113T+L149R+V258P+A276S+A364G+S388K;

V45L+L149R+G187K+V258P+A276S+A364G+S388K;

M28P+V45L+L149R+V258P+A276S+A364G+S388K;

V45L+L149R+S173Y+V258P+A276S+A364G+S388K;

A25W+V45L+L149R+V258P+A276S+Q314A+A364G;

V45L+L149R+G187L+V258P+A276S+H358T+A364G;

V45L+L149R+T215V+V258P+A276S+H358T+A364G;

V45L+T70R+L149R+V258P+A276S+A364G+S388K;

V45L+L149R+V258P+A276S+V350L+H358T+A364G;

D9E+V45L+L149R+V258P+A276S+H358T+A364G;

V45L+L149R+T211H+V258P+A276S+H358T+A364G;

V45L+L149R+V258P+A276S+L281V+A364G+S388K;

V45L+L149R+V258P+A276S+S282N+H358T+A364G;

V45L+L149R+T215I+V258P+A276S+H358T+A364G;

A25W+V45L+L149R+S173Y+V258P+A276S+A364G;

V45L+Q61K+L149R+V258P+A276S+A364G+S388K;

A25W+V45L+Q61K+L149R+V258P+A276S+A364G;

V45L+L149R+S214E+V258P+A276S+L281V+A364G;

V45 L+L149R+V258P+A276S+A286E+H358T+A364G;

D9E+V45L+L149R+V258P+A276S+A364G+S388K;

V45L+S92D+L149R+V258P+A276S+A364G+S388K;

A25W+V45L+L149R+V258P+A276S+A286E+A364G;

V45L+L149R+N168G+V258P+A276S+L281V+A364G;

V45L+Q61R+L149R+V258P+A276S+H358T+A364G;

V45L+L149R+G187S+V258P+A276S+A364G+S388K;

V45L+L149R+V258P+A276S+L281V+Q314A+A364G;

V45L+L149R+V258P+A276S+L281V+H358T+A364G;

V45L+L149R+A170S+V258P+A276S+H358T+A364G;

V45L+L149R+A170S+V258P+A276S+A364G+S388K;

V45L+L149R+T215V+V258P+A276S+A364G+S388K;

V45L+L149R+V258P+A276Q+H358T+A364G+S388K;

A25W+V45N+L149R+S214E+V258P+A276S+A364G;

V45 L+L149R+V258P+A276S+A286S+H358T+A364G;

A25W+V45L+L149R+G187S+V258P+A276S+A364G;

V45L+L149R+T180Q+V258P+A276S+L281V+A364G;

V45L+S92D+L149R+V258P+A276S+L281V+A364G;

V45L+Q61R+L149R+V258P+A276S+L281V+A364G;

V45L+L149R+A170S+V258P+A276S+L281V+A364G;

D11E+A25W+V45L+L149R+V258P+A276S+A364G;

A25W+V45L+Q61R+L149R+V258P+A276S+A364G;

A25W+V45L+L149R+T215V+V258P+A276S+A364G;

D9E+V45L+L149R+V258P+A276S+L281V+A364G;

A25W+V45L+L149R+V258P+A276S+V350L+A364G;

V45L+L149R+H219V+V258P+A276S+L281V+A364G;

V45L+L149R+T180Q+V258P+A276S+H358T+A364G;

V45L+L149R+T215V+V258P+A276S+L281V+A364G;

A25W+V45L+L149R+V258P+A276S+S282N+A364G;

A25W+V45L+L149R+T215I+V258P+A276S+A364G;

A25W+V45L+L149R+H219I+V258P+A276S+A364G;

V45L+A117S+L149R+V258P+A276S+L281V+A364G;

D9E+A25W+V45L+L149R+V258P+A276S+A364G;

V45L+L149R+S214Y+V258P+A276S+A364G+S388K;

A25W+V45L+L149R+T180Q+V258P+A276S+A364G;

A25W+V45L+L149R+V258P+A276S+L281V+A364G;

V45L+N127D+L149R+V258P+A276S+L281V+A364G;

V45L+L149R+S173Y+V258P+A276S+H358T+A364G;

V45L+Q61R+L149R+V258P+A276S+A364G+S388K;

V45 L+L149R+V258P+A276S+L281V+A286E+A364G;

A25W+V45L+L149R+W250Y+V258P+A276S+A364G;

A25W+V45L+L149R+V258P+N260H+A276S+A364G;

V45 L+L149R+V258P+A276S+L281V+V350L+A364G;

A25W+V45L+L149R+A170S+V258P+A276S+A364G;

V45 L+L149R+V258P+A276S+L281V+A286S+A364G;

V45L+L149R+V258P+A276S+L281V+S282N+A364G;

D11E+V45L+L149R+V258P+A276S+L281V+A364G;

V45L+Q61K+L149R+V258P+A276S+L281V+A364G;

V45L+L149R+T215I+V258P+A276S+L281V+A364G;

A25W+V45L+S92D+L149R+V258P+A276S+A364G;

V45L+L149R+S214Y+V258P+A276S+L281V+A364G;

V45L+L149R+T211H+V258P+A276S+L281V+A364G;

A25W+V45L+L149R+G187L+V258P+A276S+A364G;

V45L+S92D+L149R+V258P+A276S+H358T+A364G;

V45L+L149R+G187K+V258P+A276S+H358T+A364G;

V45L+L149R+N163H+V258P+A276S+L281V+A364G;

A25W+V45L+T70R+L149R+V258P+A276S+A364G;

V45L+L149R+S173Y+V258P+A276S+L281V+A364G;

V45L+L149R+G187L+V258P+A276S+L281V+A364G;

V45L+L149R+G187K+V258P+A276S+L281V+A364G;

V45 L+L149R+W250Y+V258P+A276S+H358T+A364G;

V45L+L149R+T211H+V258P+A276S+A364G+S388K;

V45L+L149R+N163H+V258P+A276S+A364G+S388K;

V45 L+L149R+V258P+A276S+A286S+A364G+S388K;

A25W+V45L+L149R+S214Y+V258P+A276S+A364G;

A25W+V45L+D113T+L149R+V258P+A276S+A364G;

V45L+T70R+L149R+V258P+A276S+L281V+A364G;

A25W+V45L+N127D+L149R+V258P+A276S+A364G;

V45 L+T70R+L149R+V258P+A276S+H358T+A364G;

V45L+L149R+H219I+V258P+A276S+L281V+A364G;

A25W+V45L+A117S+L149R+V258P+A276S+A364G;

A25W+V45L+L149R+N168G+V258P+A276S+A364G;

V45L+N127D+L149R+V258P+A276S+H358T+A364G;

A25W+V45L+L149R+H219V+V258P+A276S+A364G;

D11E+V45L+L149R+V258P+A276S+H358T+A364G;

V45L+L149R+G187S+V258P+A276S+L281V+A364G;

A25W+V45L+L149R+N163H+V258P+A276S+A364G;

A25W+V45L+L149R+G187K+V258P+A276S+A364G;

V45L+L149R+V258P+N260H+A276S+H358T+A364G;

V45L+D113T+L149R+V258P+A276S+L281V+A364G;

V45L+D113T+L149R+V258P+A276S+H358T+A364G;

V45L+L149R+N168G+V258P+A276S+H358T+A364G;

V45L+L149R+S214Y+V258P+A276S+H358T+A364G;

V45L+A117S+L149R+V258P+A276S+H358T+A364G;

A25W+M28P+V45L+L149R+V258P+A276S+A364G;

M28P+V45L+L149R+V258P+A276S+L281V+A364G;

M28P+V45L+L149R+V258P+A276S+H358T+A364G;

V45L+L149R+N163H+V258P+A276S+H358T+A364G;

V45L+L149R+T215K+V258P+A276S+H358T+A364G;

V45L+L149R+H219I+V258P+A276S+A364G+S388K;

V45L+L149R+T215K+V258P+A276S+A364G+S388K;

V45L+L149R+W250Y+V258P+A276S+L281V+A364G;

V45L+L149R+H219V+V258P+A276S+A364G+S388K;

A25W+V45L+L149R+V258P+A276S+N355G+A364G;

V45L+L149R+V258P+A276S+N355G+A364G+S388K;

V45L+L149R+V258P+A276S+L281V+N355G+A364G;

V45 L+L149R+V258P+A276S+N355G+H358T+A364G;

V45L+L149R+H219V+V258P+A276S+H358T+A364G;

V45L+A79C+L149R+S214E+V258P+A276S+A364G+S388K;

V45L+A79C+L149R+S214Q+V258P+A276S+A364G+S388K;

V45L+A79C+L149R+V258P+A276S+Q314A+A364G+S388K;

V45L+A79C+L149R+V258P+A276S+L281V+A364G+S388K;

V45L+A79C+L149R+S173Y+V258P+A276S+A364G+S388K;

V45L+A79C+L149R+G187K+V258P+A276S+A364G+S388K;

V45L+Q61R+A79C+L149R+V258P+A276S+A364G+S388K;

V45L+A79C+L149R+N168G+V258P+A276S+A364G+S388K;

V45L+A79C+L149R+T215V+V258P+A276S+A364G+S388K;

V45L+A79C+L149R+V258P+A276S+A286S+A364G+S388K;

V45L+A79C+L149R+H219V+V258P+A276S+A364G+S388K;

V45L+A79C+L149R+A170S+V258P+A276S+A364G+S388K;

D11E+V45L+A79C+L149R+V258P+A276S+A364G+S388K;

V45L+A79C+L149R+V258P+A276S+A286E+A364G+S388K;

V45L+A79C+L149R+T180Q+V258P+A276S+A364G+S388K;

V45L+A79C+L149R+H219I+V258P+A276S+A364G+S388K;

V45L+L149R+S214E+V258P+A276S+H358D+A364G+D470T;

V45L+A79C+L149R+V258P+A276S+V350L+A364G+S388K;

V45L+L149R+S214E+V258P+A276S+H358D+A364G+D470T;

V45L+A79C+D113T+L149R+V258P+A276S+A364G+S388K;

V45L+A79C+N127D+L149R+V258P+A276S+A364G+S388K;

D9E+V45L+A79C+L149R+V258P+A276S+A364G+S388K;

V45L+L149R+S214E+V258P+A276S+H358D+A364G+D470T;

V45L+A79C+L149R+W250Y+V258P+A276S+A364G+S388K;

V45L+L149R+S214E+V258P+A276S+H358D+A364G+D470T;

V45L+L149R+S214E+V258P+A276S+H358D+A364G+D470T;

V45L+A79C+L149R+N163H+V258P+A276S+A364G+S388K;

V45L+A117S+L149R+S214Q+V258P+A276S+H358D+A364G;

A25W+V45L+A79C+L149R+S214E+V258P+A276S+A364G;

V45L+A117S+L149R+S214Q+V258P+A276S+A364G+D470T;

V45L+A79C+S92D+L149R+V258P+A276S+A364G+S388K;

V45L+A117S+L149R+S214Q+V258P+A276S+H358D+A364G;

V45L+L149R+S214Q+V258P+A276S+A286E+A364G+S388K;

V45L+L149R+S214Q+T215I+V258P+A276S+A364G+S388K;

A25W+V45L+L149R+S214Q+V258P+A276S+A364G+S388K;

V45L+L149R+A170S+S214Q+V258P+A276S+A364G+S388K;

V45L+Q61K+L149R+S214Q+V258P+A276S+A364G+S388K;

V45L+L149R+N168G+S214Q+V258P+A276S+A364G+S388K;

V45L+A79C+D95D+L149R+V258P+A276S+A364G+S388K;

V45L+Q77E+A79C+L149R+V258P+A276S+A364G+S388K;

V45L+L149R+S214Q+V258P+A276S+S282N+A364G+S388K;

V45L+L149R+S214Q+V258P+A276S+Q314A+A364G+S388K;

V45L+T70R+L149R+S214Q+V258P+A276S+A364G+S388K;

V45L+L149R+S214Q+W250Y+V258P+A276S+A364G+S388K;

V45L+L149R+G187K+S214Q+V258P+A276S+A364G+S388K;

V45L+L149R+V258P+A276S+L281V+H358T+A364G+S388K;

M28P+V45L+L149R+S214Q+V258P+A276S+A364G+S388K;

V45L+L149R+G187L+S214Q+V258P+A276S+A364G+S388K;

V45L+L149R+S214Q+V258P+A276S+A286S+A364G+S388K;

D9E+V45L+L149R+S214Q+V258P+A276S+A364G+S388K;

V45L+L149R+S173Y+S214Q+V258P+A276S+A364G+S388K;

V45L+A79C+L149R+V258P+A276S+H358T+A364G+S388K;

A25W+V45L+L149R+S214E+V258P+A276S+H358T+A364G;

A25W+V45L+L149R+S214E+V258P+A276S+A286E+A364G;

A25W+V45L+Q61K+L149R+S214E+V258P+A276S+A364G;

A25W+V45L+L149R+V258P+A276S+H358T+A364G+S388K;

V45L+L149R+S214Q+V258P+A276S+V350L+A364G+S388K;

D11E+V45L+L149R+S214Q+V258P+A276S+A364G+S388K;

V45L+L149R+H219I+V258P+A276S+H358T+A364G+S388K;

V45L+S92D+L149R+S214Q+V258P+A276S+A364G+S388K;

A25W+V45L+L149R+T211H+S214E+V258P+A276S+A364G;

A25W+V45L+L149R+S214E+H219V+V258P+A276S+A364G;

V45L+L149R+W250Y+V258P+A276S+H358T+A364G+S388K;

D9E+V45L+L149R+V258P+A276S+H358T+A364G+S388K;

V45L+L149R+H219V+V258P+A276S+H358T+A364G+S388K;

V45L+A117S+L149R+S214E+V258P+A276S+A364G+S388K;

D11E+A25W+V45L+L149R+S214E+V258P+A276S+A364G;

V45L+L149R+V258P+A276S+Q314A+H358T+A364G+S388K;

V45L+A117S+L149R+S214Q+V258P+A276S+A364G+S388L;

V45L+L149R+T180Q+S214Q+V258P+A276S+A364G+S388K;

V45L+D113T+L149R+S214Q+V258P+A276S+A364G+S388K;

A25W+V45L+Q61R+L149R+S214E+V258P+A276S+A364G;

A25W+V45L+L149R+S214E+V258P+A276S+L281V+A364G;

D9E+A25W+V45L+L149R+S214E+V258P+A276S+A364G;

V45L+Q61R+L149R+V258P+A276S+H358T+A364G+S388K;

V45L+D113T+L149R+V258P+A276S+H358T+A364G+S388K;

V45L+A117S+L149R+S214Q+V258P+A276S+A364G+S388R;

V45L+Q61R+L149R+S214Q+V258P+A276S+A364G+S388K;

V45L+A117S+L149R+S214Q+V258P+A276Q+A364G+S388K;

V45L+A117S+L149R+S214Q+V258P+A276S+A364G+S388K;

V45L+L149R+T180Q+V258P+A276S+H358T+A364G+S388K;

A25W+V45L+S92D+L149R+S214E+V258P+A276S+A364G;

A25W+V45L+L149R+S214E+V258P+A276S+V350L+A364G;

V45L+L149R+T211H+S214Q+V258P+A276S+A364G+S388K;

A25W+V45L+L149R+S214E+H219I+V258P+A276S+A364G;

V45L+L149R+T211H+V258P+A276S+H358T+A364G+S388K;

A25W+V45L+A117S+L149R+S214E+V258P+A276S+A364G;

A25W+V45L+L149R+S214E+T215V+V258P+A276S+A364G;

V45L+A117S+L149R+V258P+A276S+H358T+A364G+S388K;

V45L+L149R+N168G+V258P+A276S+H358T+A364G+S388K;

V45L+A117S+L149R+S214Q+V258P+A276S+A364G+S388H;

A25W+V45L+L149R+S214E+T215I+V258P+A276S+A364G;

V45L+N127D+L149R+V258P+A276S+H358T+A364G+S388K;

V45L+A117S+L149R+S214Q+V258P+A276S+A364G+S388A;

V45N+A117S+L149R+S214Q+V258P+A276S+A364G+S388K;

A25W+V45L+L149R+G187S+S214E+V258P+A276S+A364G;

V45L+A117S+L149R+S214Y+V258P+A276S+A364G+S388K;

A25W+V45L+L149R+S214E+V258P+A276S+A286S+A364G;

V45L+L149R+V258P+A276S+A286S+H358T+A364G+S388K;

V45L+L149R+S173Y+V258P+A276S+H358T+A364G+S388K;

V45L+A79C+L149R+V258P+A276S+N355G+A364G+S388K;

A25W+V45L+L149R+G187L+S214E+V258P+A276S+A364G;

V45L+L149R+S214Q+V258P+A276S+L281V+A364G+S388K;

V45L+L149R+S214Q+T215V+V258P+A276S+A364G+S388K;

V45L+L149R+N163H+V258P+A276S+H358T+A364G+S388K;

V45L+A79C+A117S+L149R+V258P+A276S+A364G+S388K;

V45L+L149R+A170S+V258P+A276S+H358T+A364G+S388K;

A25W+V45L+L149R+S214E+T215K+V258P+A276S+A364G;

V45L+S92D+L149R+V258P+A276S+H358T+A364G+S388K;

V45L+L149R+S214Q+V258P+A276S+N355G+A364G+S388K;

A25W+V45L+L149R+S214E+V258P+A276S+N355G+A364G;

A25W+V45L+L149R+S214E+V258P+A276S+Q314A+A364G;

V45L+A117S+L149R+S214Q+V258P+A276S+A364G+S388R+D470T;

V45L+A117S+L149R+S214Q+V258P+A276S+A364G+S388K+D470T;

V45L+A117S+L149R+S214Q+V258P+A276S+A364G+S388L+D470T;

V45L+A117S+L149R+S214Q+V258P+A276Q+A364G+S388K+D470T;

V45L+A117S+L149R+D192E+S214Q+V258P+A276S+H358D+A364G;

V45L+A117S+L149R+S214Q+V258P+A276S+A364G+S388A+D470T;

V45L+A117S+L149R+S214Q+V258P+A276S+A364G+S388H+D470T;

V45N+A117S+L149R+S214Q+V258P+A276S+A364G+S388K+D470T;

V45L+A117S+L149R+S214E+V258P+A276S+H358D+A364G+S388K;

V45L+A117S+L149R+D192E+S214Q+V258P+A276S+H358D+A364G;

V45L+A117S+L149R+S214Q+V258P+A276S+H358D+A364G+S388K;

V45L+A117S+L149R+S214Q+V258P+A276S+A364G+S388K+R483V;

V45L+A117S+L149R+S214E+V258P+A276S+A364G+S388K+D470T;

V45L+A117S+L149R+S214Q+V258P+A276S+H358D+A364G+S388R;

V45L+A117S+L149R+S214Q+V258P+A276S+H358D+A364G+S388A;

V45L+A117S+L149R+S214Q+V258P+A276S+A364G+Q384A+S388K;

V45L+A117S+L149R+S214Q+V258P+A276S+H358D+A364G+S388L;

V45L+A117S+L149R+S214Q+V258P+A276S+A286E+A364G+S388K;

V45L+A117S+L149R+S214Q+V258P+A276S+L281V+A364G+S388K;

V45L+A117S+L149R+S214Q+V258P+A276S+A364G+S388K+G394D;

V45L+A117S+L149R+S214Q+V258P+A276S+A364G+S388K+S473G;

V45L+A117S+L149R+S214Q+V258P+A276S+A364G+S388K+G394A;

V45N+A117S+L149R+S214Q+V258P+A276S+H358D+A364G+S388K;

V45L+A117S+L149R+S214Q+V258P+A276S+A364G+S388K+L478P;

V45L+A117S+L149R+S214Q+V258P+A276S+A364G+S388K+S481A;

V45L+A117S+L149R+S214Q+V258P+A276S+Q314A+A364G+S388K;

V45L+A117S+L149R+S214Q+V258P+A276Q+H358D+A364G+S388K;

V45L+A117S+L149R+G187K+S214Q+V258P+A276S+A364G+S388K;

V45L+A117S+L149R+S214Q+V258P+A276S+A364G+S388K+L478N;

V45L+A117S+L149R+S214Q+V258P+A276S+A364G+S388K+V393R;

V45L+A117S+L149R+S214Q+V258P+A276S+A364G+T366V+S388K;

V45L+A117S+L149R+S214Q+V258P+A276S+H358T+A364G+S388K;

D11E+V45L+A117S+L149R+S214Q+V258P+A276S+A364G+S388K;

V45L+A117S+L149R+S214Q+V258P+A276S+A364G+S388K+G394Q;

V45L+A117S+L149R+S214Q+V258P+A276S+A364G+S388K+N395D;

V45L+A117S+L149R+S214Q+V258P+A276S+A364G+S388K+V393H;

V45L+A117S+L149R+S214Q+V258P+A276S+A364G+S388K+S501E;

V45L+A117S+L149R+S214Q+V258P+A276S+A364G+S388K+S528H;

V45L+A117S+L149R+S214Q+V258P+A276S+H358D+A364G+S388H;

V45L+A117S+L149R+S214Q+V258P+A276S+A364G+S388K+S389H;

V45L+A117S+L149R+S214Q+V258P+A276S+A364G+T366I+S388K;

V45L+A117S+L149R+S214Q+V258P+A276S+A364G+S388K+N474G;

V45L+A117S+L149R+S214Y+V258P+A276S+H358D+A364G+S388K;

V45L+A117S+L149R+S214Q+V258P+A276S+A286S+A364G+S388K;

V45L+A117S+L149R+S214Q+V258P+A276S+A364G+S388K+A499Y;

V45L+A117S+L149R+S214Q+V258P+A276S+A364G+S388K+V500V;

V45L+A117S+L149R+S214Q+W250Y+V258P+A276S+A364G+S388K;

V45L+A117S+L149R+S214Q+V258P+A276S+A364G+S388K+T480N;

V45L+A117S+L149R+G187S+S214Q+V258P+A276S+A364G+S388K;

A25W+V45L+A117S+L149R+S214Q+V258P+A276S+A364G+S388K;

V45L+A117S+L149R+S214Q+V258P+A276S+A364G+S388K+N476N;

V45L+A117S+L149R+S214Q+V258P+A276S+A364G+S388K+N476D;

D9E+V45L+A117S+L149R+S214Q+V258P+A276S+A364G+S388K;

V45L+A117S+L149R+S214Q+V258P+A276S+A364G+S388K+S481S;

V45L+A117S+L149R+S214Q+H219V+V258P+A276S+A364G+S388K;

V45L+A117S+L149R+S214Q+T215I+V258P+A276S+A364G+S388K;

V45L+A117S+L149R+S214Q+V258P+A276S+A364G+S388K+Q496R;

V45L+A117S+L149R+A170S+S214Q+V258P+A276S+A364G+S388K;

V45L+Q61R+A117S+L149R+S214Q+V258P+A276S+A364G+S388K;

V45L+A117S+L149R+S214Q+V258P+A276S+A364G+S388K+V497P;

V45L+A117S+L149R+T180Q+S214Q+V258P+A276S+A364G+S388K;

V45L+A117S+L149R+S214Q+T215V+V258P+A276S+A364G+S388K;

V45L+A117S+L149R+S214Q+V258P+A276S+V350L+A364G+S388K;

V45L+Q61K+A117S+L149R+S214Q+V258P+A276S+A364G+S388K;

V45L+A117S+L149R+S214Q+V258P+A276S+A364G+S388K+Y391V;

V45L+A117S+L149R+N168G+S214Q+V258P+A276S+A364G+S388K;

V45L+D113T+A117S+L149R+S214Q+V258P+A276S+A364G+S388K;

M28P+V45L+A117S+L149R+S214Q+V258P+A276S+A364G+S388K;

V45L+A117S+L149R+S214Q+V258P+A276S+A364G+S388K+T477K;

V45L+A117S+L149R+S214Q+V258P+A276S+A364G+S388K+S389V;

V45L+A117S+L149R+S173Y+S214Q+V258P+A276S+A364G+S388K;

V45L+A117S+L149R+S214Q+V258P+A276S+A364G+S388K+V500T;

V45L+A117S+L149R+T211H+S214Q+V258P+A276S+A364G+S388K;

V45L+S92D+A117S+L149R+S214Q+V258P+A276S+A364G+S388K;

V45L+A117S+L149R+S214Q+V258P+A276S+A364G+S388K+A472L;

V45L+A117S+L149R+S214Q+V258P+A276S+A364G+S388K+S473K;

V45L+A117S+L149R+S214Q+V258P+A276S+A364G+S388K+A499L;

V45L+A117S+L149R+S214Q+V258P+A276S+A364G+S388K+V498I;

V45L+A117S+L149R+N163H+S214Q+V258P+A276S+A364G+S388K;

V45L+A117S+L149R+S214Q+V258P+A276S+A364G+S388K+N527H;

V45L+A117S+L149R+S214Q+V258P+A276S+A364G+S388K+T477V;

V45L+A117S+L149R+S214Q+V258P+A276S+A364G+S388K+G503L;

V45L+A117S+L149R+G187L+S214Q+V258P+A276S+A364G+S388K;

V45L+A117S+L149R+S214Q+T215K+V258P+A276S+A364G+S388K;

V45L+A117S+L149R+S214Q+V258P+A276S+A364G+Q385C+S388K;

V45L+A117S+L149R+S214Q+V258P+A276S+N355G+A364G+S388K;

V45L+A117S+L149R+S214E+V258P+A276S+H358D+A364G+S388K+D470T;

V45L+A117S+L149R+S214Q+V258P+A276S+H358D+A364G+S388R+D470T;

V45L+A117S+L149R+S214Q+V258P+A276Q+H358D+A364G+S388K+D470T;

V45L+A117S+L149R+S214Q+V258P+A276S+H358D+A364G+S388H+D470T;

V45L+A79C+A117S+L149R+S214Q+V258P+A276S+H358D+A364G+S388K;

V45L+A117S+L149R+S214Q+V258P+A276S+H358D+A364G+S388L+D470T;

V45L+A117S+L149R+S214E+V258P+A276S+A364G+S388K+D470T+R483V;

V45L+A117S+L149R+S214Q+V258P+A276S+A364G+S388R+D470T+R483V;

V45L+A117S+L149R+S214Q+V258P+A276S+A364G+S388K+D470A+R483V;

V45L+A117S+L149R+S214Q+V258P+A276Q+A364G+S388K+D470T+R483V;

V45L+A117S+L149R+S214Q+V258P+A276S+A364G+S388K+D470T+R483V;

V45L+A117S+L149R+S214Q+V258P+A276S+H358D+A364G+S388K+D470T;

V45N+A117S+L149R+S214Q+V258P+A276S+A364G+S388K+D470T+R483V;

V45L+A117S+L149R+S214Q+V258P+A276S+A364G+S388H+D470T+R483V;

V45L+A79C+A117S+L149R+S214Q+V258P+A276S+A364G+S388K+D470T;

V45L+A117S+L149R+S214Q+V258P+A276S+H358D+A364G+S388A+D470T;

V45L+A117S+L149R+S214Q+V258P+A276S+A364G+S388A+D470T+R483V;

V45L+A117S+L149R+S214Q+V258P+A276S+L281V+A364G+S388K+D470T;

V45L+A117S+L149R+S214Q+V258P+A276S+A364G+Q384A+S388K+D470T;

V45L+A117S+L149R+S214Q+V258P+A276S+A286E+A364G+S388K+D470T;

V45L+A117S+L149R+S214Q+V258P+A276S+A364G+S388K+D470T+T477K;

V45L+A117S+L149R+S214Y+V258P+A276S+H358D+A364G+S388K+D470T;

V45L+A117S+L149R+S214Q+V258P+A276S+A364G+S388K+G394A+D470T;

V45L+A117S+L149R+S214Q+V258P+A276S+A364G+S388K+D470T+N476D;

V45L+A117S+L149R+S214Q+V258P+A276S+A364G+S388K+V393H+D470T;

V45L+A117S+L149R+S214Q+V258P+A276S+A364G+S388K+N395D+D470T;

V45L+A117S+L149R+S214Q+V258P+A276S+H358T+A364G+S388K+D470T;

V45L+A117S+L149R+S214Q+V258P+A276S+A364G+S388K+D470T+L478N;

V45L+A117S+L149R+S214Q+W250Y+V258P+A276S+A364G+S388K+D470T;

V45L+A117S+L149R+G187L+S214Q+V258P+A276S+A364G+S388K+D470T;

D9E+V45L+A117S+L149R+S214Q+V258P+A276S+A364G+S388K+D470T;

V45L+A117S+L149R+S214Q+V258P+A276S+S282N+A364G+S388K+D470T;

D11E+V45L+A117S+L149R+S214Q+V258P+A276S+A364G+S388K+D470T;

V45L+A117S+L149R+S214Q+V258P+A276S+A364G+S388K+D470T+N476N;

V45L+A117S+L149R+S214Q+V258P+A276S+A364G+S388K+G394D+D470T;

V45L+Q61K+A117S+L149R+S214Q+V258P+A276S+A364G+S388K+D470T;

V45L+A117S+L149R+S214Q+V258P+A276S+A286S+A364G+S388K+D470T;

V45L+A117S+L149R+S214Q+V258P+A276S+H358D+A364G+S388K+D470A;

V45L+A117S+L149R+S214Q+V258P+A276S+A364G+S388K+G394Q+D470T;

V45L+A117S+L149R+S214Q+V258P+A276S+A364G+S388K+D470T+L478P;

V45L+A117S+L149R+S214Q+V258P+A276S+V350L+A364G+S388K+D470T;

V45L+D113T+A117S+L149R+S214Q+V258P+A276S+A364G+S388K+D470T;

V45L+A117S+L149R+T180Q+S214Q+V258P+A276S+A364G+S388K+D470T;

V45L+A117S+L149R+S214Q+V258P+A276S+A364G+S388K+S389H+D470T;

V45L+A117S+L149R+S214Q+V258P+A276S+A364G+S388K+V393R+D470T;

V45L+A117S+L149R+S214Q+V258P+A276S+A364G+S388K+D470T+Q496R;

V45L+A117S+N127D+L149R+S214Q+V258P+A276S+A364G+S388K+D470T;

V45 L+A117S+L149R+S214Q+V258P+A276S+A364G+S388K+D470T+A499Y;

V45L+A117S+L149R+S173Y+S214Q+V258P+A276S+A364G+S388K+D470T;

V45L+S92D+A117S+L149R+S214Q+V258P+A276S+A364G+S388K+D470T;

V45L+A117S+L149R+S214Q+V258P+A276S+A364G+S388K+D470T+N529T;

V45L+A117S+L149R+S214Q+V258P+A276S+H358D+A364G+S388K+L478N;

V45L+T70R+A117S+L149R+S214Q+V258P+A276S+A364G+S388K+D470T;

M28P+V45L+A117S+L149R+S214Q+V258P+A276S+A364G+S388K+D470T;

V45L+A117S+L149R+S214Y+V258P+A276S+A364G+S388K+D470T+R483V;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

V45L+Q61R+A117S+L149R+S214Q+V258P+A276S+A364G+S388K+D470T;

V45L+A117S+L149R+S214Q+T215I+V258P+A276S+A364G+S388K+D470T;

V45L+A117S+L149R+S214Q+V258P+A276S+A364G+S388K+D470T+Q496E;

V45L+A117S+L149R+G187K+S214Q+V258P+A276S+A364G+S388K+D470T;

V45L+A117S+L149R+S214Q+V258P+A276S+A364G+S388K+D470T+W495I;

V45L+A117S+L149R+S214Q+V258P+A276S+A364G+S388K+D470T+V498I;

V45 L+A117S+L149R+S214Q+V258P+A276S+A364G+S388K+D470T+V500V;

D11E+V45L+A117S+L149R+S214Q+V258P+A276S+H358D+A364G+S388K;

V45L+A117S+L149R+A170S+S214Q+V258P+A276S+A364G+S388K+D470T;

V45L+A117S+L149R+S214Q+V258P+A276S+A364G+S388K+Y391V+D470T;

V45L+A117S+L149R+S214Q+V258P+A276S+Q314A+A364G+S388K+D470T;

V45L+A117S+L149R+S214Q+V258P+A276S+A364G+S388K+D470T+T477V;

V45L+A117S+L149R+S214Q+V258P+A276S+H358D+A364G+S388K+G394Q;

V45L+A117S+L149R+S214Q+H219V+V258P+A276S+A364G+S388K+D470T;

V45L+A117S+L149R+S214Q+V258P+A276S+A364G+S388K+D470T+Q496Q;

V45L+A117S+L149R+S214Q+V258P+A276S+H358D+A364G+S388K+A499Y;

V45L+A117S+L149R+S214Q+T215V+V258P+A276S+A364G+S388K+D470T;

V45L+A117S+L149R+S214Q+V258P+A276S+A364G+Q385C+S388K+D470T;

V45 L+A117S+L149R+S214Q+V258P+A276S+A364G+S388K+D470T+T480N;

V45 L+A117S+L149R+S214Q+V258P+A276S+A364G+S388K+D470T+V497P;

V45L+A117S+L149R+S214Q+V258P+A276S+A364G+S388K+D470T+A499L;

V45L+A117S+L149R+S214Q+V258P+A276S+A364G+S388K+D470T+S501N;

V45L+A117S+L149R+S214Q+V258P+A276S+H358D+A364G+Q384A+S388K;

V45L+A117S+L149R+S214Q+V258P+A276S+A364G+S388L+D470T+R483V;

V45L+A117S+L149R+S214Q+V258P+A276S+H358D+A364G+S388K+G394A;

V45L+A117S+L149R+S214Q+V258P+A276S+H358D+A364G+S388K+V498I;

V45L+A117S+L149R+S214Q+H219I+V258P+A276S+A364G+S388K+D470T;

V45L+A117S+L149R+N163H+S214Q+V258P+A276S+A364G+S388K+D470T;

V45L+A117S+L149R+S214Q+V258P+A276S+Q314A+H358D+A364G+S388K;

V45L+A117S+L149R+S173Y+S214Q+V258P+A276S+H358D+A364G+S388K;

V45L+A117S+L149R+S214Q+V258P+A276S+H358D+A364G+S388K+G394D;

V45L+Q61R+A117S+L149R+S214Q+V258P+A276S+H358D+A364G+S388K;

V45L+A117S+L149R+S214Q+V258P+A276S+H358D+A364G+T366V+S388K;

V45L+A117S+L149R+T180Q+S214Q+V258P+A276S+H358D+A364G+S388K;

V45L+A117S+L149R+S214Q+T215I+V258P+A276S+H358D+A364G+S388K;

V45L+A117S+L149R+S214Q+W250Y+V258P+A276S+H358D+A364G+S388K;

V45L+A117S+L149R+S214Q+V258P+A276S+H358D+A364G+S388K+N476D;

V45L+A117S+L149R+S214Q+V258P+A276S+H358D+A364G+S388K+T477V;

V45L+A117S+L149R+S214Q+V258P+A276S+H358D+A364G+S388K+L478P;

V45L+A117S+L149R+S214Q+V258P+A276S+H358D+A364G+S388K+S481A;

V45L+A117S+L149R+T211H+S214Q+V258P+A276S+A364G+S388K+D470T;

V45L+A117S+L149R+S214Q+V258P+A276S+H358D+A364G+S388K+V500T;

V45L+A117S+L149R+S214Q+V258P+A276S+V350L+H358D+A364G+S388K;

V45L+A117S+L149R+G187K+S214Q+V258P+A276S+H358D+A364G+S388K;

V45L+A117S+L149R+S214Q+V258P+A276S+A364G+S388K+D470V+R483V;

V45L+A117S+L149R+S214Q+V258P+A276S+H358D+A364G+S388K+V500V;

V45L+A117S+L149R+N163H+S214Q+V258P+A276S+H358D+A364G+S388K;

V45L+A117S+L149R+S214Q+V258P+A276S+H358D+A364G+S388K+S481S;

V45L+A117S+L149R+S214Q+V258P+A276S+A364G+S388K+D470T+V500T;

V45L+A117S+L149R+S214Q+V258P+A276S+H358D+A364G+S388K+W495S;

V45L+D113T+A117S+L149R+S214Q+V258P+A276S+H358D+A364G+S388K;

V45L+A117S+L149R+S214Q+V258P+A276S+H358D+A364G+S388K+N474G;

V45L+A117S+L149R+S214Q+V258P+A276S+S282N+H358D+A364G+S388K;

V45L+A117S+L149R+S214Q+V258P+A276S+H358D+A364G+S388K+Q496Q;

V45L+A117S+L149R+S214Q+V258P+A276S+A364G+S388K+S389V+D470T;

V45L+A117S+L149R+S214Q+V258P+A276S+H358D+A364G+S388K+S501E;

V45L+A117S+L149R+S214Q+V258P+A276S+H358D+A364G+S388K+N476N;

V45L+A117S+L149R+S214Q+V258P+A276S+H358D+A364G+S388K+S473K;

V45L+A117S+L149R+G187L+S214Q+V258P+A276S+H358D+A364G+S388K;

V45L+A117S+L149R+S214Q+V258P+A276S+H358D+A364G+S388K+Q496E;

A25W+V45L+A117S+L149R+S214Q+V258P+A276S+H358D+A364G+S388K;

V45L+A117S+L149R+N168G+S214Q+V258P+A276S+H358D+A364G+S388K;

V45L+A117S+L149R+S214Q+V258P+A276S+H358D+A364G+S388K+S389H;

V45L+A117S+L149R+S214Q+V258P+A276S+A364G+S388K+D470T+T480L;

V45L+A117S+L149R+S214Q+V258P+A276S+H358D+A364G+Q385C+S388K;

V45L+S92D+A117S+L149R+S214Q+V258P+A276S+H358D+A364G+S388K;

V45L+A117S+L149R+A170S+S214Q+V258P+A276S+H358D+A364G+S388K;

V45L+A117S+L149R+S214Q+V258P+A276S+H358D+A364G+S388K+Y391V;

V45L+A117S+L149R+S214Q+V258P+A276S+H358D+A364G+S388K+T480N;

D9E+V45L+A117S+L149R+S214Q+V258P+A276S+H358D+A364G+S388K;

V45L+A117S+L149R+S214Q+H219I+V258P+A276S+H358D+A364G+S388K;

V45L+A117S+L149R+S214Q+V258P+A276S+H358D+A364G+S388K+A499L;

V45L+A117S+L149R+S214Q+T215V+V258P+A276S+H358D+A364G+S388K;

V45L+A117S+L149R+S214Q+H219V+V258P+A276S+H358D+A364G+S388K;

V45L+A117S+L149R+S214Q+V258P+A276S+H358D+A364G+S388K+S389V;

V45L+A117S+L149R+S214Q+V258P+A276S+H358D+A364G+S388K+V393H;

V45L+A117S+N127D+L149R+S214Q+V258P+A276S+H358D+A364G+S388K;

V45L+A117S+L149R+S214Q+V258P+A276S+H358D+A364G+S388K+A472L;

V45L+A117S+L149R+T211H+S214Q+V258P+A276S+H358D+A364G+S388K;

V45L+A117S+L149R+S214Q+V258P+A276S+H358D+A364G+S388K+T480L;

V45L+A117S+L149R+S214Q+V258P+A276S+H358D+A364G+S388K+V393R;

V45L+A117S+L149R+S214Q+V258P+A276S+A286S+H358D+A364G+S388K;

V45L+A117S+L149R+S214Q+V258P+A276S+N355G+H358D+A364G+S388K;

V45L+A117S+L149R+S214Q+V258P+A276S+N355G+A364G+S388K+D470T;

V45L+A117S+L149R+S214Q+V258P+A276S+A286E+H358D+A364G+S388K;

V45L+A117S+L149R+S214Q+V258P+A276S+H358D+A364G+Y386G+S388K;

V45L+A117S+L149R+S214Q+V258P+A276S+A364G+S388K+D470T+S501E;

V45L+A79C+A117S+L149R+S214Q+V258P+A276S+A364G+S388K+D470T+R483V;

V45L+A79C+A117S+L149R+S214Q+V258P+A276S+H358D+A364G+S388K+D470T;

V45L+A117S+L149R+S214E+V258P+A276S+H358D+A364G+S388K+D470T+R483V;

V45L+A117S+L149R+S214Q+V258P+A276S+H358D+A364G+S388K+D470T+R483V;

V45L+A117S+L149R+S214E+V258P+A276S+H358D+A364G+S388K+D470T+L530R;

V45L+A117S+L149R+S214Q+V258P+A276Q+H358D+A364G+S388K+D470T+R483V;

V45L+A117S+L149R+S214Q+V258P+A276Q+H358D+A364G+S388K+D470T+L530R;

V45L+A117S+L149R+S214Q+V258P+A276S+H358D+A364G+S388K+D470T+L530R;

V45L+A117S+L149R+S214Q+V258P+A276S+H358D+A364G+S388K+D470A+R483V;

V45N+A117S+L149R+S214Q+V258P+A276S+H358D+A364G+S388K+D470T+R483V;

V45L+A117S+L149R+S214Q+V258P+A276S+H358D+A364G+S388H+D470T+L530R;

V45N+A117S+L149R+S214Q+V258P+A276S+H358D+A364G+S388K+D470T+L530R;

V45L+A117S+L149R+S214Q+V258P+A276S+H358D+A364G+S388A+D470T+L530R;

V45L+A117S+L149R+S214Q+V258P+A276S+H358D+A364G+S388A+D470T+R483V;

V45L+A117S+L149R+S214Q+V258P+A276S+H358D+A364G+S388L+D470T+R483V;

V45L+Q61K+A117S+L149R+S214Q+V258P+A276S+H358D+A364G+S388K+D470T;

V45L+A117S+L149R+S214Q+V258P+A276S+A364G+S388K+G394A+D470T+R483V;

V45L+A117S+L149R+S214Q+V258P+A276S+H358D+A364G+S388K+D470T+L478N;

V45L+A117S+L149R+S214Q+V258P+A276S+L281V+H358D+A364G+S388K+D470T;

V45L+A117S+L149R+S214Q+V258P+A276S+H358D+A364G+T366I+S388K+D470T;

V45L+A117S+L149R+S214Q+V258P+A276S+H358D+A364G+S388K+D470T+S473G;

V45L+A117S+L149R+S214Q+V258P+A276S+H358D+A364G+S388K+D470T+L478P;

V45L+A117S+L149R+S214Q+V258P+A276S+H358D+A364G+S388K+G394D+D470T;

V45L+A117S+L149R+S214Q+V258P+A276S+A364G+Q384A+S388K+D470T+R483V;

V45L+A117S+L149R+S214Q+V258P+A276S+H358D+A364G+S388K+D470T+N529T;

V45L+A117S+L149R+S214Q+V258P+A276S+H358D+A364G+S388K+G394Q+D470T;

V45L+A117S+L149R+G187L+S214Q+V258P+A276S+H358D+A364G+S388K+D470T;

V45L+A117S+L149R+S214Q+V258P+A276S+Q314A+H358D+A364G+S388K+D470T;

V45L+A117S+L149R+S214Q+V258P+A276S+V350L+H358D+A364G+S388K+D470T;

V45L+A117S+L149R+G187K+S214Q+V258P+A276S+H358D+A364G+S388K+D470T;

V45L+A117S+L149R+S214Q+W250Y+V258P+A276S+H358D+A364G+S388K+D470T;

V45L+A117S+L149R+S214Q+V258P+A276S+A286E+H358D+A364G+S388K+D470T;

V45L+A117S+L149R+S214Q+V258P+A276S+H358D+A364G+Q384A+S388K+D470T;

V45L+A117S+L149R+S214Q+V258P+A276S+H358D+A364G+S388K+D470T+S528H;

V45L+A117S+L149R+S214Q+V258P+A276S+H358D+A364G+S388K+D470T+Q496R;

V45L+A117S+L149R+N163H+S214Q+V258P+A276S+H358D+A364G+S388K+D470T;

V45L+A117S+L149R+S214Q+V258P+A276S+S282N+H358D+A364G+S388K+D470T;

D11E+V45L+A117S+L149R+S214Q+V258P+A276S+H358D+A364G+S388K+D470T;

V45L+A117S+L149R+S214Q+V258P+A276S+H358D+A364G+T366V+S388K+D470T;

V45L+A117S+L149R+S214Q+V258P+A276S+H358D+A364G+S388K+D470T+S473K;

V45L+A117S+L149R+S214Q+V258P+A276S+H358D+A364G+S388R+D470T+R483V;

V45L+A117S+L149R+S214Q+V258P+A276S+Q314A+A364G+S388K+D470T+R483V;

V45L+S92D+A117S+L149R+S214Q+V258P+A276S+H358D+A364G+S388K+D470T;

V45L+A117S+L149R+S214Q+V258P+N260H+A276S+H358D+A364G+S388K+D470T;

V45L+A117S+L149R+G187S+S214Q+V258P+A276S+H358D+A364G+S388K+D470T;

V45L+A117S+L149R+S214Q+V258P+A276S+H358D+A364G+S388K+D470T+T477K;

V45L+A117S+L149R+S214Q+V258P+A276S+H358D+A364G+S388K+D470T+N474G;

V45L+A117S+L149R+S214Q+V258P+A276S+H358D+A364G+S388K+N395D+D470T;

V45L+A117S+L149R+S214Q+V258P+A276S+H358D+A364G+S388K+D470T+S501N;

V45L+A117S+N127D+L149R+S214Q+V258P+A276S+H358D+A364G+S388K+D470T;

V45L+A117S+L149R+S214Q+V258P+A276S+H358D+A364G+S388K+V393R+D470T;

V45L+A117S+L149R+S173Y+S214Q+V258P+A276S+H358D+A364G+S388K+D470T;

V45L+A117S+L149R+S214Q+V258P+A276S+H358D+A364G+S388K+D470T+T480N;

V45L+A117S+L149R+N168G+S214Q+V258P+A276S+H358D+A364G+S388K+D470T;

V45L+Q61K+A117S+L149R+S214Q+V258P+A276S+A364G+S388K+D470T+R483V;

V45L+D113T+A117S+L149R+S214Q+V258P+A276S+H358D+A364G+S388K+D470T;

V45 L+A117S+L149R+S214Q+V258P+A276S+A364G+T366V+S388K+D470T+R483V;

V45L+A117S+L149R+S214Q+V258P+A276S+H358T+A364G+S388K+D470T+R483V;

V45L+A117S+L149R+S214Q+V258P+A276S+A364G+S388K+V393R+D470T+R483V;

V45L+A117S+L149R+S214Q+V258P+A276S+H358D+A364G+S388K+D470T+Q496Q;

V45L+A117S+L149R+S214Q+V258P+A276S+A364G+S388K+G394Q+D470T+R483V;

V45L+A117S+L149R+S214Q+V258P+A276S+A364G+T366I+S388K+D470T+R483V;

V45L+A117S+L149R+S214Q+V258P+A276S+H358D+A364G+S388K+D470T+L484S;

V45L+A117S+L149R+S214Q+V258P+A276S+H358D+A364G+S388K+D470T+A499L;

V45L+A117S+L149R+S214Q+V258P+A276S+H358T+A364G+S388K+D470T+L530R;

V45L+A117S+L149R+S214Q+V258P+A276S+H358D+A364G+S388K+D470T+V498I;

V45L+T70R+A117S+L149R+S214Q+V258P+A276S+A364G+S388K+D470T+R483V;

V45L+A117S+L149R+S214Q+V258P+A276S+A364G+S388K+D470T+N476D+R483V;

D9E+V45L+A117S+L149R+S214Q+V258P+A276S+A364G+S388K+D470T+R483V;

V45L+A117S+L149R+S214Q+V258P+A276S+H358D+A364G+S388K+D470T+S501E;

V45L+A117S+L149R+S214Q+V258P+A276S+H358D+A364G+S388K+D470T+S481A;

V45L+A117S+L149R+S214Q+H219V+V258P+A276S+H358D+A364G+S388K+D470T;

V45L+A117S+L149R+S214Q+V258P+A276S+H358D+A364G+S388K+D470T+S481S;

V45L+A117S+L149R+G187K+S214Q+V258P+A276S+A364G+S388K+D470T+R483V;

V45L+A117S+L149R+S214Q+V258P+A276S+A364G+S388K+N395D+D470T+R483V;

V45L+A117S+L149R+G187L+S214Q+V258P+A276S+A364G+S388K+D470T+R483V;

V45L+A117S+L149R+S214Q+V258P+A276S+H358D+A364G+S388K+D470T+V497P;

V45L+A117S+L149R+S214Q+V258P+A276S+H358D+A364G+S388K+D470T+N527H;

V45L+A117S+L149R+S214Q+V258P+A276S+A364G+S388K+D470T+R483V+Q496Q;

V45L+A117S+L149R+N168G+S214Q+V258P+A276S+A364G+S388K+D470T+R483V;

V45L+A117S+L149R+S214Q+V258P+A276S+A364G+S388K+D470T+N476N+R483V;

V45L+A117S+L149R+S214Q+V258P+A276S+A364G+S388K+D470T+T477V+R483V;

V45L+A117S+L149R+S214Q+V258P+A276S+V350L+A364G+S388K+D470T+R483V;

V45L+A117S+L149R+S214Q+V258P+A276S+A364G+S388K+D470T+L478N+R483V;

M28P+V45L+A117S+L149R+S214Q+V258P+A276S+H358D+A364G+S388K+D470T;

V45L+A117S+L149R+S214Q+V258P+A276S+A364G+S388K+V393H+D470T+R483V;

V45L+Q61R+A117S+L149R+S214Q+V258P+A276S+A364G+S388K+D470T+R483V;

V45L+A117S+L149R+S214Q+V258P+A276S+A364G+S388K+D470T+R483V+V497P;

V45L+A117S+L149R+S214Q+V258P+A276S+A364G+S388K+D470T+R483V+V498I;

V45L+A117S+L149R+S214Q+V258P+A276S+A286E+A364G+S388K+D470T+R483V;

V45L+A117S+L149R+S214Q+V258P+A276S+A364G+S388K+D470T+T477K+R483V;

V45L+A117S+L149R+S214Q+V258P+A276S+A364G+S388K+D470T+R483V+Q496E;

V45L+A117S+L149R+S214Q+V258P+A276S+A364G+S388K+D470T+R483V+Q496R;

V45L+A117S+L149R+S214Q+V258P+A276S+A364G+S388K+D470T+R483V+A499L;

V45L+A117S+L149R+S214Q+T215I+V258P+A276S+H358D+A364G+S388K+D470T;

V45L+A117S+L149R+S214Q+V258P+A276S+A364G+S388K+Y391V+D470T+R483V;

V45L+A117S+L149R+S214Q+V258P+A276S+H358D+A364G+S388K+D470T+N476D;

V45L+A117S+L149R+S214Q+V258P+A276S+H358D+A364G+S388K+V393H+D470T;

V45L+A117S+L149R+S214Q+V258P+A276S+A286S+A364G+S388K+D470T+R483V;

V45L+A117S+L149R+S214Q+V258P+A276S+A364G+S388K+D470T+R483V+S501E;

V45L+A117S+L149R+S214Q+V258P+A276S+A364G+S388K+D470T+R483V+N529T;

V45L+A117S+L149R+S214Q+V258P+A276S+A364G+S388K+D470T+R483V+S501N;

V45L+A117S+L149R+S214Q+V258P+A276S+A364G+S388K+D470T+S481S+R483V;

V45L+A117S+L149R+S214Q+V258P+A276S+A364G+S388K+D470T+L478P+R483V;

D11E+V45L+A117S+L149R+S214Q+V258P+A276S+A364G+S388K+D470T+R483V;

V45L+A117S+L149R+S214Q+V258P+A276S+H358D+A364G+S388K+D470T+T477V;

V45L+A117S+L149R+T211H+S214Q+V258P+A276S+H358D+A364G+S388K+D470T;

V45L+A117S+L149R+S214Y+V258P+A276S+H358D+A364G+S388K+D470T+R483V;

V45L+A117S+L149R+S214Q+W250Y+V258P+A276S+A364G+S388K+D470T+R483V;

V45L+S92D+A117S+L149R+S214Q+V258P+A276S+A364G+S388K+D470T+R483V;

V45L+A117S+L149R+S214Q+V258P+A276S+A364G+S388K+D470T+R483V+N527H;

V45L+A117S+L149R+S214Q+V258P+A276S+H358D+A364G+Q385C+S388K+D470T;

V45L+A117S+L149R+S173Y+S214Q+V258P+A276S+A364G+S388K+D470T+R483V;

V45L+A117S+L149R+S214Q+V258P+A276S+A364G+S388K+S389H+D470T+R483V;

V45L+A117S+L149R+S214Q+H219V+V258P+A276S+A364G+S388K+D470T+R483V;

V45L+A117S+L149R+S214Q+V258P+A276S+H358D+A364G+S388K+D470T+Q496E;

V45L+A117S+L149R+A170S+S214Q+V258P+A276S+H358D+A364G+S388K+D470T;

V45L+A117S+L149R+S214Q+V258P+A276S+H358D+A364G+S388K+S389V+D470T;

V45L+T70R+A117S+L149R+S214Q+V258P+A276S+H358D+A364G+S388K+D470T;

V45L+A117S+L149R+S214Q+H219I+V258P+A276S+A364G+S388K+D470T+R483V;

V45 L+A117S+L149R+S214Q+V258P+A276S+A364G+S388K+D470T+R483V+A499Y;

V45L+A117S+L149R+S214Q+V258P+A276S+L281V+A364G+S388K+D470T+R483V;

V45L+A117S+L149R+S214Q+V258P+A276S+A364G+S388K+D470T+R483V+V500T;

V45L+A117S+L149R+T180Q+S214Q+V258P+A276S+H358D+A364G+S388K+D470T;

V45L+A117S+L149R+S214Q+V258P+A276S+H358D+A364G+S388K+D470T+V500V;

V45L+A117S+L149R+S214Q+V258P+A276S+H358D+A364G+S388K+D470T+G526T;

V45L+A117S+L149R+S214Q+V258P+A276S+A286S+H358D+A364G+S388K+D470T;

V45L+A117S+L149R+S214Q+V258P+A276S+H358D+A364G+S388K+D470A+L530R;

V45L+A117S+L149R+S214Q+V258P+A276S+A364G+S388K+D470T+R483V+D525L;

V45L+A117S+L149R+S214Q+V258P+A276S+H358D+A364G+S388K+D470T+N476N;

V45L+A117S+L149R+S214Q+V258P+A276S+H358D+A364G+S388K+S389H+D470T;

V45L+A117S+L149R+S214Y+V258P+A276S+H358D+A364G+S388K+D470T+L530R;

A25W+V45L+A117S+L149R+S214Q+V258P+A276S+H358D+A364G+S388K+D470T;

V45L+A117S+L149R+S214Q+T215V+V258P+A276S+A364G+S388K+D470T+R483V;

V45L+Q61R+A117S+L149R+S214Q+V258P+A276S+H358D+A364G+S388K+D470T;

V45L+A117S+L149R+S214Q+H219I+V258P+A276S+H358D+A364G+S388K+D470T;

V45L+A117S+L149R+T211H+S214Q+V258P+A276S+A364G+S388K+D470T+R483V;

V45L+D95N+A117S+L149R+S214Q+V258P+A276S+H358D+A364G+S388K+S473G;

V45L+A117S+L149R+S214Q+V258P+A276S+H358D+A364G+S388K+D470T+G503L;

V45L+A117S+L149R+S214Q+V258P+A276S+H358D+A364G+S388K+D470T+D525L;

V45L+A117S+L149R+S214Q+V258P+A276S+H358D+A364G+S388K+D470T+V500T;

V45L+A117S+L149R+S214Q+T215V+V258P+A276S+H358D+A364G+S388K+D470T;

V45L+A117S+L149R+S214Q+V258P+A276S+H358D+A364G+S388K+D470T+T480L;

V45L+A117S+L149R+S214Q+V258P+A276S+A364G+S388K+S389V+D470T+R483V;

V45 L+A117S+L149R+S214Q+V258P+A276S+A364G+S388K+D470T+R483V+V497E;

V45L+A117S+L149R+S214Q+V258P+A276S+H358D+A364G+S388K+D470V+R483V;

V45L+A117S+L149R+S214Q+V258P+A276S+H358D+A364G+S388K+D470K+R483V;

V45L+A117S+L149R+S214Q+V258P+A276S+H358D+A364G+S388K+D470T+A472L;

V45L+D113T+A117S+L149R+S214Q+V258P+A276S+A364G+S388K+D470T+R483V;

V45L+A117S+L149R+S214Q+T215K+V258P+A276S+A364G+S388K+D470T+R483V;

V45L+A117S+L149R+S214Q+V258P+A276S+A364G+S388K+D470T+R483V+V497W;

V45L+A117S+L149R+S214Q+V258P+A276S+A364G+S388K+D470T+R483V+W495E;

V45L+A117S+L149R+S214Q+T215K+V258P+A276S+H358D+A364G+S388K+D470T;

V45L+A117S+L149R+S214Q+V258P+A276S+H358D+A364G+S388H+D470T+R483V;

V45L+A117S+L149R+S214Q+V258P+A276S+N355G+H358D+A364G+S388K+D470T;

V45L+A79C+A117S+L149R+S214Q+V258P+A276S+H358D+A364G+S388K+D470T+L 530R;

V45L+A79C+A117S+L149R+S214Q+V258P+A276S+H358D+A364G+S388K+D470T+R 483V;

V45L+A117S+L149R+S214Q+V258P+A276S+H358D+A364G+Q385C+S388K+D470T+R483V;

V45L+Q61K+A117S+L149R+S214Q+V258P+A276S+H358D+A364G+S388K+D470T+R 483V;

V45L+A117S+L149R+S214Q+V258P+A276S+L281V+H358D+A364G+S388K+D470T+R483V;

V45L+A117S+L149R+S214Q+V258P+A276S+H358D+A364G+T366I+S388K+D470T+R 483V;

V45L+A117S+L149R+S214Q+V258P+A276S+H358D+A364G+Q384A+S388K+D470T+L530R;

V45L+Q61K+A117S+L149R+S214Q+V258P+A276S+H358D+A364G+S388K+D470T+L 530R;

V45L+T70R+A117S+L149R+S214Q+V258P+A276S+H358D+A364G+S388K+D470T+R 483V;

V45L+A117S+L149R+S214Q+V258P+A276S+H358D+A364G+S388K+V393H+D470T+R483V;

V45L+A117S+L149R+S214Q+V258P+A276S+H358D+A364G+S388K+G394A+D470T+R483V;

V45L+A117S+L149R+S214Q+V258P+A276S+H358D+A364G+S388K+V393H+D470T+L530R;

V45L+A117S+L149R+S214Q+V258P+A276S+A286E+H358D+A364G+S388K+D470T+R483V;

D11E+V45L+A117S+L149R+S214Q+V258P+A276S+H358D+A364G+S388K+D470T+R 483V;

V45L+A117S+L149R+G187L+S214Q+V258P+A276S+H358D+A364G+S388K+D470T+L530R;

V45L+A117S+L149R+S214Q+V258P+A276S+H358D+A364G+Q384A+S388K+D470T+V45L+A117S+L149R+G187K+S214Q+V258P+A276S+H358D+A364G+S388K+D470T+R483V;

V45L+A117S+L149R+S214Q+V258P+A276S+L281V+H358D+A364G+S388K+D470T+L530R;

V45L+A117S+L149R+G187L+S214Q+V258P+A276S+H358D+A364G+S388K+D470T+R483V;

V45L+A117S+L149R+S214Q+V258P+A276S+H358D+A364G+S388K+G394Q+D470T+R483V;

V45L+A117S+L149R+S214Q+V258P+A276S+H358D+A364G+S388K+G394A+D470T+L530R;

V45L+A117S+L149R+S214Q+V258P+A276S+H358D+A364G+T366V+S388K+D470T+L530R;

V45L+A117S+L149R+S214Q+V258P+A276S+H358D+A364G+S388K+G394Q+D470T+L530R;

V45L+A117S+L149R+S214Q+V258P+A276S+S282N+H358D+A364G+S388K+D470T+R483V;

V45L+A117S+L149R+S214Q+V258P+A276S+H358D+A364G+S388K+N395D+D470T+R483V;

V45L+A117S+L149R+S214Q+V258P+A276S+H358D+A364G+S388K+D470T+N476D+R483V;

V45L+A117S+L149R+S214Q+V258P+A276S+V350L+H358D+A364G+S388K+D470T+R483V;

V45L+A117S+L149R+S214Q+V258P+A276S+H358D+A364G+S388K+D470T+T477K+R483V;

V45L+A117S+L149R+S214Q+V258P+A276S+H358D+A364G+S388K+D470T+R483V+Q496R;

V45L+A117S+L149R+S214Q+V258P+A276S+H358D+A364G+S388K+D470T+R483V+W495E;

V45L+A117S+L149R+S214Q+V258P+A276S+A286E+H358D+A364G+S388K+D470T+L530R;

V45L+A117S+L149R+G187K+S214Q+V258P+A276S+H358D+A364G+S388K+D470T+L530R;

V45L+A117S+L149R+S214Q+V258P+A276S+H358D+A364G+S388K+Y391V+D470T+R483V;

D9E+V45L+A117S+L149R+S214Q+V258P+A276S+H358D+A364G+S388K+D470T+L5 30R;

V45L+A117S+L149R+S214Q+V258P+A276S+H358D+A364G+S388K+D470T+T477V+R483V;

V45L+A117S+L149R+S214Q+V258P+A276S+H358D+A364G+S388K+D470T+N476N+R483V;

V45L+A117S+L149R+S214Q+V258P+A276S+H358D+A364G+S388K+V393R+D470T+R483V;

V45L+A117S+L149R+S214Q+V258P+A276S+H358D+A364G+S388K+D470T+T480L+L530R;

V45L+A117S+L149R+S214Q+V258P+A276S+H358D+A364G+S388K+D470T+S481A+L530R;

V45L+A117S+L149R+S214Q+W250Y+V258P+A276S+H358D+A364G+S388K+D470T+R483V;

V45L+A117S+L149R+S214Q+V258P+A276S+H358D+A364G+S388K+D470T+S481S+R483V;

V45L+A117S+L149R+S173Y+S214Q+V258P+A276S+H358D+A364G+S388K+D470T+R483V;

V45L+A117S+L149R+T180Q+S214Q+V258P+A276S+H358D+A364G+S388K+D470T+L530R;

D11E+V45L+A117S+L149R+S214Q+V258P+A276S+H358D+A364G+S388K+D470T+L 530R;

V45L+A117S+L149R+S214Q+V258P+A276S+H358D+A364G+S388K+D470T+N476D+L530R;

V45L+A117S+L149R+S214Q+V258P+A276S+A286S+H358D+A364G+S388K+D470T+R483V;

V45L+A117S+L149R+S214Q+V258P+A276S+H358D+A364G+S388K+D470T+R483V+Q496Q;

V45L+A117S+L149R+S214Q+V258P+A276S+H358D+A364G+S388K+D470T+R483V+Q496E;

V45L+Q61R+A117S+L149R+S214Q+V258P+A276S+H358D+A364G+S388K+D470T+L 530R;

V45L+A117S+L149R+S214Q+V258P+A276S+Q314A+H358D+A364G+S388K+D470T+L530R;

V45L+A117S+L149R+S214Q+V258P+A276S+H358D+A364G+S388K+D470T+L478P+R483V;

A25W+V45L+A117S+L149R+S214Q+V258P+A276S+H358D+A364G+S388K+D470T+V45L+A117S+L149R+T180Q+S214Q+V258P+A276S+H358D+A364G+S388K+D470T+R483V;

V45L+A117S+L149R+S214Q+V258P+A276S+H358D+A364G+S388K+D470T+R483V+L484K;

V45L+A117S+L149R+S214Q+V258P+A276S+H358D+A364G+S388K+D470T+N474G+L530R;

V45L+A117S+L149R+S214Q+V258P+A276S+H358D+A364G+S388K+N395D+D470T+L530R;

V45L+A117S+L149R+S214Q+V258P+A276S+H358D+A364G+T366V+S388K+D470T+R483V;

V45L+A117S+L149R+N168G+S214Q+V258P+A276S+H358D+A364G+S388K+D470T+R483V;

V45L+A117S+L149R+S214Q+V258P+A276S+H358D+A364G+S388K+D470T+R483V+L484S;

V45L+A117S+L149R+S214Q+V258P+A276S+H358D+A364G+S388K+D470T+R483V+L484 D;

V45L+A117S+L149R+S214Q+V258P+A276S+H358D+A364G+S388K+D470T+R483V+S528H;

V45L+A117S+L149R+S214Q+V258P+A276S+H358D+A364G+S388K+D470T+R483V+S501N;

V45L+A117S+L149R+S173Y+S214Q+V258P+A276S+H358D+A364G+S388K+D470T+L530R;

V45L+A117S+L149R+S214Q+V258P+A276S+H358D+A364G+S388K+D470T+R483V+A499L;

V45L+S92D+A117S+L149R+S214Q+V258P+A276S+H358D+A364G+S388K+D470T+R 483V;

V45L+A117S+L149R+S214Q+V258P+A276S+Q314A+H358D+A364G+S388K+D470T+R483V;

V45L+A117S+L149R+S214Q+V258P+A276S+N355G+H358D+A364G+S388K+D470T+L530R;

V45L+A117S+L149R+S214Q+V258P+A276S+V350L+H358D+A364G+S388K+D470T+L530R;

V45L+A117S+L149R+S214Q+V258P+A276S+H358D+A364G+S388K+D470T+S481S+L530R;

V45L+A117S+L149R+S214Q+V258P+A276S+H358D+A364G+S388K+D470T+Q496E+L530R;

V45L+A117S+L149R+N168G+S214Q+V258P+A276S+H358D+A364G+S388K+D470T+L530R;

V45L+A117S+L149R+S214Q+V258P+A276S+H358D+A364G+S388K+D470T+V498I+L 530R;

V45L+A117S+L149R+S214Q+V258P+A276S+H358D+A364G+S388K+D470T+R483V+A499Y;

V45L+A117S+L149R+S214Q+V258P+A276S+A286S+H358D+A364G+S388K+D470T+L530R;

V45L+A117S+L149R+S214Q+V258P+A276S+H358D+A364G+S388K+D470T+S501N+L530R;

V45L+A117S+L149R+S214Q+V258P+A276S+H358D+A364G+S388K+D470T+N476N+L530R;

V45L+A117S+L149R+S214Q+V258P+A276S+H358D+A364G+S388K+D470T+T477V+L530R;

V45L+A117S+L149R+S214Q+V258P+A276S+H358D+A364G+S388K+D470T+L478N+L530R;

V45L+S92D+A117S+L149R+S214Q+V258P+A276S+H358D+A364G+S388K+D470T+L 530R;

V45L+A117S+L149R+S214Q+V258P+A276S+H358D+A364G+S388K+D470T+V500V+L530R;

V45L+A117S+L149R+S214Q+V258P+A276S+H358D+A364G+S388K+D470T+R483V+V500V;

V45L+A117S+L149R+S214Q+V258P+A276S+H358D+A364G+S388K+D470T+L478P+L530R;

V45L+A117S+L149R+S214Q+V258P+A276S+H358D+A364G+S388K+D470T+R483V+S501E;

V45L+A117S+L149R+S214Q+V258P+A276S+H358D+A364G+S388K+D470T+D525L+L530R;

V45L+A117S+L149R+S214Q+V258P+A276S+H358D+A364G+S388K+D470T+G526T+L530R;

V45L+A117S+L149R+S214Q+V258P+A276S+H358D+A364G+S388K+D470T+N527H+L530R;

V45L+A117S+L149R+S214Q+V258P+A276S+H358D+A364G+S388K+D470T+T477K+L530R;

M28P+V45L+A117S+L149R+S214Q+V258P+A276S+H358D+A364G+S388K+D470T+L 530R;

D9E+V45L+A117S+L149R+S214Q+V258P+A276S+H358D+A364G+S388K+D470T+R4 83V;

V45L+A117S+L149R+S214Q+V258P+A276S+H358D+A364G+S388K+D470T+S501E+L530R;

V45L+A117S+L149R+S214Q+W250Y+V258P+A276S+H358D+A364G+S388K+D470T+L530R;

V45L+A117S+L149R+S214Q+V258P+A276S+H358D+A364G+S388K+D470T+A499L+L530R;

V45L+A117S+L149R+S214Q+V258P+A276S+H358D+A364G+S388K+D470T+A472I+L 530R;

V45L+A117S+N127D+L149R+S214Q+V258P+A276S+H358D+A364G+S388K+D470T+R483V;

V45L+A117S+L149R+S214Q+V258P+A276S+H358D+A364G+S388K+D470T+V500T+L530R;

V45L+A117S+L149R+S214Q+V258P+A276S+H358D+A364G+S388K+D470T+R483V+N527H;

V45L+A117S+L149R+S214Q+V258P+A276S+H358D+A364G+S388K+D470T+T480N+L530R;

V45L+A117S+L149R+S214Q+V258P+A276S+H358D+A364G+S388K+D470T+R483V+V497P;

V45L+A117S+L149R+S214Q+V258P+A276S+H358D+A364G+S388K+D470T+R483V+V498I;

V45L+A117S+L149R+S214Q+V258P+A276S+H358D+A364G+S388K+D470T+T480N+R483V;

V45L+A117S+L149R+S214Q+H219V+V258P+A276S+H358D+A364G+S388K+D470T+L530R;

M28P+V45L+A117S+L149R+S214Q+V258P+A276S+H358D+A364G+S388K+D470T+R483V;

V45L+A117S+L149R+A170S+S214Q+V258P+A276S+H358D+A364G+S388K+D470T+R483V;

V45L+A117S+L149R+S214Q+V258P+A276S+H358D+A364G+S388K+S389H+D470T+L530R;

V45L+D113T+A117S+L149R+S214Q+V258P+A276S+H358D+A364G+S388K+D470T+L530R;

V45L+A117S+L149R+N163H+S214Q+V258P+A276S+H358D+A364G+S388K+D470T+V45L+A117S+L149R+T211H+S214Q+V258P+A276S+H358D+A364G+S388K+D470T+L530R;

V45L+A117S+L149R+S214Q+V258P+A276S+H358D+A364G+S388K+D470T+R483V+V500T;

V45L+D113T+A117S+L149R+S214Q+V258P+A276S+H358D+A364G+S388K+D470T+R483V;

V45L+D95N+A117S+L149R+S214Q+V258P+A276S+H358D+A364G+S388K+G394A+D470T;

V45L+A93T+A117S+L149R+S214Q+V258P+A276S+H358D+A364G+S388K+D470T+A 499Y;

V45L+A117S+L149R+S214Q+T215I+V258P+A276S+H358D+A364G+S388K+D470T+R 483V;

V45L+A117S+L149R+S214Q+H219I+V258P+A276S+H358D+A364G+S388K+D470T+R483V;

V45L+A117S+L149R+S214Q+V258P+A276S+H358D+A364G+Q385C+S388K+D470T+L530R;

V45L+A117S+L149R+S214Q+V258P+A276S+H358D+A364G+S388K+D470T+R483V+L530R;

V45L+A117S+L149R+S214Q+V258P+A276S+H358D+A364G+S388K+D470T+R483V+G503L;

V45L+A117S+L149R+S214Q+T215V+V258P+A276S+H358D+A364G+S388K+D470T+R483V;

V45L+A117S+L149R+S214Q+V258P+A276S+H358D+A364G+S388K+D470T+S481A+R483V;

V45L+A117S+L149R+S214Q+T215V+V258P+A276S+H358D+A364G+S388K+D470T+L530R;

V45L+A117S+L149R+G187S+S214Q+V258P+A276S+H358D+A364G+S388K+D470T+L530R;

V45L+A117S+L149R+T211H+S214Q+V258P+A276S+H358D+A364G+S388K+D470T+R483V;

V45L+A117S+L149R+S214Q+V258P+A276S+H358D+A364G+S388K+D470T+R483V+G526T;

V45L+A117S+L149R+S214Q+V258P+A276S+H358D+A364G+S388K+D470T+R483V+D525L;

V45L+A117S+L149R+S214Q+V258P+A276S+H358D+A364G+S388K+D470T+S528H+L530R;

V45L+A117S+L149R+S214Q+V258P+A276S+H358D+A364G+S388K+I469T+D470T+R 483V;

V45L+A117S+L149R+S214Q+V258P+A276S+H358D+A364G+S388K+D470T+N529T+L530R;

V45L+A117S+L149R+S214Q+V258P+A276S+S282N+H358D+A364G+S388K+D470T+L530R;

V45L+A117S+L149R+S214Q+V258P+A276S+H358D+A364G+S388K+D470T+G503L+L530R;

V45L+A117S+L149R+S214Q+H219I+V258P+A276S+H358D+A364G+S388K+D470T+L 530R;

V45L+A117S+L149R+S214Q+V258P+A276S+H358D+A364G+S388K+D470T+V497P+L530R;

V45L+A117S+L149R+S214Q+T215I+V258P+A276S+H358D+A364G+S388K+D470T+L 530R;

V45L+A117S+L149R+S214Q+V258P+A276S+H358D+A364G+S388K+S389V+D470T+R483V;

V45L+A117S+L149R+S214Q+V258P+A276S+H358D+A364G+S388K+S389V+D470T+L530R;

V45L+A117S+L149R+S214Q+V258P+A276S+H358D+A364G+S388K+D470T+R483V+V497D;

V45L+A117S+L149R+S214Q+V258P+A276S+H358D+A364G+S388K+D470T+L478N+R483V;

V45L+T70R+D95N+A117S+L149R+S214Q+V258P+A276S+L356M+H358D+A364G+S3 88K;

V45L+A117S+L149R+S214Q+T215K+V258P+A276S+H358D+A364G+S388K+D470T+R483V;

V45L+A117S+L149R+S214Q+V258P+A276S+H358D+A364G+S388K+D470T+R483V+V497W;

V45L+T70R+A117S+L149R+S214Q+V258P+A276S+H358D+A364G+S388K+D470T+L 530R;

V45L+A117S+L149R+S214Q+T215K+V258P+A276S+H358D+A364G+S388K+D470T+L530R;

V45L+Q61R+A117S+L149R+S214Q+V258P+A276S+H358D+A364G+S388K+D470T+R483V;

V45L+A117S+L149R+S214Q+V258P+A276S+N355G+H358D+A364G+S388K+D470T+V45L+A117S+L149R+S214Q+V258P+A276S+H358D+A364G+S388K+D470T+R483V+V497E;

V45L+A117S+L149R+N163H+S214Q+V258P+A276S+H358D+A364G+S388K+D470T+L530R;

V45 L+A117S+L149R+S214Q+V258P+A276S+H358 D+A364G+Y386G+S388K+D470T+R483V;

V45L+A117S+L149R+S214Q+V258P+A276S+H358D+A364G+S388K+G394D+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+T70R+A117S+L149R+S214Q+V258P+A276S+L281V+H358D+A364G+S388K+G 394A+D470T+R483V;

V45L+Q61K+A117S+L149R+S214Q+V258P+A276S+L281V+H358D+A364G+S388K+G 394A+D470T+L530R;

V45L+Q61K+A117S+L149R+S214Q+V258P+A276S+L281V+H358D+A364G+S388K+D 470T+R483V+L530R;

V45L+Q61K+A117S+L149R+S214Q+V258P+A276S+L281V+H358D+A364G+S388K+D 470T+R483V+L530R;

V45L+T70R+A117S+L149R+S214Q+V258P+A276S+L281V+H358D+A364G+S388K+G 394A+D470T+R483V;

V45L+Q61K+A117S+L149R+S214Q+V258P+A276S+L281V+H358D+A364G+S388K+G 394A+D470T+R483V+L530R;

V45L+Q61K+A117S+L149R+S214Q+V258P+A276S+L281V+H358D+A364G+S388K+G 394A+D470T+R483V+L530R;

V45L+Q61K+A117S+L149R+S214Q+V258P+A276S+L281V+H358D+A364G+S388K+G 394A+D470T+R483V+L530R;

V45L+Q61K+A117S+L149R+S214Q+V258P+A276S+L281V+H358D+A364G+S388K+G 394A+D470T+R483V+L530R;

V45L+A117S+L149R+G187K+S214Q+V258P+A276S+A286E+H358D+A364G+Q384A+S388K+V393H+D470T+R483V;

V45L+A117S+L149R+G187K+S214Q+V258P+A276S+A286E+H358D+A364G+Q384A+S388K+N395D+D470T+R483V;

V45L+A117S+L149R+G187K+S214E+T215I+V258P+A276S+A286E+H358D+A364G+S 388K+V393H+D470T+R483V;

V45L+A117S+L149R+G187K+S214E+T215I+V258P+A276S+A286E+H358D+A364G+S 388K+N395D+D470T+R483V;

V45L+T70R+A117S+L149R+S214Q+V258P+A276S+L281V+H358D+A364G+S388K+G 394A+C468V+D470T+R483V;

V45L+T70R+A117S+L149R+S214Q+V258P+A276S+L281V+H358D+A364G+S388K+G 394A+0468A+D470T+R483V;

V45L+T70R+A117S+L149R+S214Q+C243V+V258P+A276S+L281V+H358D+A364G+S 388K+G394A+D470T+R483V;

V45L+T70R+A117S+L149R+S214Q+V258P+A276S+L281V+H358D+A364G+S388K+G 394A+C468L+D470T+R483V;

V45L+T70R+A117S+L149R+S214Q+V258P+A276S+L281V+H358D+A364G+C375V+S 388K+G394A+D470T+R483V;

V45L+T70R+A117S+L149R+S214Q+V258P+A276S+L281V+H358D+A364G+S388K+G 394A+C468S+D470T+R483V;

V45L+C60L+T70R+A117S+L149R+S214Q+V258P+A276S+L281V+H358D+A364G+S3 88K+G394A+D470T+R483V;

V45L+C69L+T70R+A117S+L149R+S214Q+V258P+A276S+L281V+H358D+A364G+S3 88K+G394A+D470T+R483V;

V45L+C60A+T70R+A117S+L149R+S214Q+V258P+A276S+L281V+H358D+A364G+S3 88K+G394A+D470T+R483V;

V45L+T70R+A117S+L149R+S214Q+C243L+V258P+A276S+L281V+H358D+A364G+S 388K+G394A+D470T+R483V;

V45L+C60S+T70R+A117S+L149R+S214Q+V258P+A276S+L281V+H358D+A364G+S3 88K+G394A+D470T+R483V;

V45L+C69A+T70R+A117S+L149R+S214Q+V258P+A276S+L281V+H358D+A364G+S3 88K+G394A+D470T+R483V;

V45L+T70R+A117S+L149R+C161S+S214Q+V258P+A276S+L281V+H358D+A364G+S 388K+G394A+D470T+R483V;

V45L+C69V+T70R+A117S+L149R+S214Q+V258P+A276S+L281V+H358D+A364G+S3 88K+G394A+D470T+R483V;

V45L+C60V+T70R+A117S+L149R+S214Q+V258P+A276S+L281V+H358D+A364G+S3 88K+G394A+D470T+R483V;

V45L+T70R+A117S+L149R+S214Q+V258P+A276S+L281V+H358D+A364G+C375A+S 388K+G394A+D470T+R483V;

V45L+T70R+A117S+L149R+S214Q+V258P+A276S+L281V+H358D+A364G+C375S+S 388K+G394A+D470T+R483V;

V45L+T70R+A117S+L149R+S214Q+V258P+A276S+L281V+H358D+A364G+S388K+G 394A+D470T+R483V+C540A;

V45L+C69S+T70R+A117S+L149R+S214Q+V258P+A276S+L281V+H358D+A364G+S3 88K+G394A+D470T+R483V;

V45L+T70R+A117S+L149R+S214Q+V258P+A276S+L281V+H358D+A364G+C375L+S 388K+G394A+D470T+R483V;

V45L+T70R+A117S+L149R+S214Q+V258P+A276S+L281V+H358D+A364G+S388K+G 394A+D470T+R483V+C540L;

V45L+T70R+A117S+L149R+S214Q+V258P+A276S+L281V+H358D+A364G+S388K+G 394A+D470T+R483V+C522S;

V45L+T70R+A117S+L149R+S214Q+V258P+A276S+L281V+H358D+A364G+S388K+G 394A+D470T+R483V+C540S;

V45L+T70R+A117S+L149R+S214Q+V258P+A276S+L281V+H358D+A364G+S388K+G 394A+C396S+D470T+R483V;

V45L+T70R+A117S+L149R+S214Q+V258P+A276S+L281V+H358D+A364G+S388K+G 394A+0396A+D470T+R483V;

C40L+V45L+T70R+A117S+L149R+S214Q+V258P+A276S+L281V+H358D+A364G+S3 88K+G394A+D470T+R483V;

C40A+V45L+T70R+A117S+L149R+S214Q+V258P+A276S+L281V+H358D+A364G+S3 88K+G394A+D470T+R483V;

C40S+V45L+T70R+A117S+L149R+S214Q+V258P+A276S+L281V+H358D+A364G+S3 88K+G394A+D470T+R483V;

C40V+V45L+T70R+A117S+L149R+S214Q+V258P+A276S+L281V+H358D+A364G+S3 88K+G394A+D470T+R483V;

V45L+T70R+A117S+L149R+S214Q+V258P+A276S+L281V+H358D+A364G+S388K+G 394A+C396V+D470T+R483V;

V45L+T70R+A117S+L149R+C161V+S214Q+V258P+A276S+L281V+H358D+A364G+S 388K+G394A+D470T+R483V;

V45L+T70R+A117S+L149R+S214Q+V258P+A276S+L281V+H358D+A364G+S388K+G 394A+C396L+D470T+R483V;

V45L+T70R+A117S+L149R+C161L+S214Q+V258P+A276S+L281V+H358D+A364G+S 388K+G394A+D470T+R483V;

V45L+T70R+A117S+L149R+S214Q+V258P+A276S+L281V+H358D+A364G+S388K+G 394A+D470T+R483V+C522A;

V45L+T70R+A117S+L149R+S214Q+C243S+V258P+A276S+L281V+H358D+A364G+S 388K+G394A+D470T+R483V;

V45L+T70R+A117S+L149R+S214Q+V258P+A276S+L281V+H358D+A364G+S388K+G 394A+D470T+R483V+C522V;

V45L+T70R+A117S+L149R+S214Q+V258P+A276S+L281V+H358D+A364G+S388K+G 394A+D470T+R483V+C540V;

V45L+Q61K+T70R+A117S+L149R+S214Q+V258P+A276S+L281V+H358D+A364G+Q3 84A+S388K+G394Q+D470T+R483V;

V45L+Q61K+T70R+A117S+L149R+S214Q+V258P+A276S+L281V+H358D+A364G+Q3 84A+S388K+G394A+D470T+R483V;

V45L+Q61K+T70R+A117S+L149R+S214Q+V258P+A276S+L281V+H358D+A364G+Q3 84A+S388K+V393H+D470T+R483V;

V45L+Q61K+T70R+A117S+L149R+S214Q+V258P+A276S+L281V+H358D+A364G+Q3 84A+S388K+V393H+D470T+R483V;

V45L+Q61K+T70R+A117S+L149R+S214Q+V258P+A276S+L281V+H358D+A364G+Q3 84A+S388K+G394A+D470T+R483V;

V45L+Q61K+T70R+A117S+L149R+S214Q+V258P+A276S+L281V+H358D+A364G+Q3 84A+S388K+V393H+D470T+R483V;

V45L+Q61K+T70R+A117S+L149R+S214Q+V258P+A276S+L281V+H358D+A364G+Q3 84A+S388K+G394A+D470T+R483V;

V45L+Q61K+T70R+A117S+L149R+S214Q+V258P+A276S+L281V+H358D+A364G+Q3 84A+S388K+V393H+D470T+R483V;

V45L+Q61K+T70R+A117S+L149R+S214Q+V258P+A276S+L281V+H358D+A364G+Q3 84A+S388K+G394A+D470T+R483V;

V45L+Q61K+T70R+A117S+L149R+S214Q+V258P+A276S+L281V+A286E+H358D+A3 64G+Q384A+S388K+V393H+D470T+R483V; or V45L+Q61K+T70R+A117S+L149R+S214Q+V258P+A276S+L281V+A286E+H358D+A3 64G+Q384A+S388K+V393H+D470T+R483V.

Paragraph 8. The xylanase variant according to any one of the preceding paragraphs, wherein the variant comprises at least one of the following substitutions or combinations of substitutions:

V45L+Q61K+T70R+A117S+L149R+S214Q+V258P+A276S+L281V+A286E+H358D+A364G+Q384A+S388K+V393H+D470T+R483V;

V45L+Q61K+T70R+A117S+L149R+S214Q+V258P+A276S+L281V+H358D+A364G+Q384A+S388K+G394Q+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+Q61K+T70R+A117S+L149R+S214Q+V258P+A276S+L281V+A286E+H358D+A364G+Q384A+S388K+V393H+D470T+R483V;

V45L+Q61K+T70R+A117S+L149R+S214Q+V258P+A276S+L281V+H358D+A364G+Q384A+S388K+V393H+D470T+R483V;

V45L+Q61K+T70R+A117S+L149R+S214Q+V258P+A276S+L281V+H358D+A364G+Q384A+S388K+G394A+D470T+R483V;

V45L+A79C+A117S+L149R+S214Q+V258P+A276S+H358D+A364G+S388K+D470T+L530R;

V45L+A79C+A117S+L149R+S214Q+V258P+A276S+H358D+A364G+S388K+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+Q61K+A117S+L149R+S214Q+V258P+A276S+L281V+H358D+A364G+S388K+G394A+D470T+R483V+L530R;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V;

V45L+Q61K+A117S+L149R+S214Q+V258P+A276S+L281V+H358D+A364G+S388K+G394A+D470T+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+D470T+R483V+L530R;

V45L+Q61K+A117S+L149R+S214Q+V258P+A276S+L281V+H358D+A364G+S388K+G394A+D470T+R483V+L530R;

V45L+Q61K+A117S+L149R+S214Q+V258P+A276S+L281V+H358D+A364G+S388K+D470T+R483V+L530R;

V45L+A117S+L149R+G187K+S214Q+V258P+A276S+A286E+H358D+A364G+Q384A+S388K+V393H+D470T+R483V;

V45L+A117S+L149R+G187K+S214Q+V258P+A276S+A286E+H358D+A364G+Q384A+S388K+N395D+D470T+R483V;

V45L+A117S+L149R+G187K+S214E+T215I+V258P+A276S+A286E+H358D+A364G+S388K+V393H+D470T+R483V;

V45L+A117S+L149R+G187K+S214E+T215I+V258P+A276S+A286E+H358D+A364G+S388K+N395D+D470T+R483V;

V45L+A117S+L149R+S214Q+V258P+A276S+H358D+A364G+Q385C+S388K+D470T+R483V;

V45L+Q61K+A117S+L149R+S214Q+V258P+A276S+H358D+A364G+S388K+D470T+R483V;

V45L+A79C+A117S+L149R+S214Q+V258P+A276S+A364G+S388K+D470T+R483V;

V45L+A117S+L149R+S214Q+V258P+A276S+L281V+H358D+A364G+S388K+D470T+R483V;

V45L+A117S+L149R+S214Q+V258P+A276S+H358D+A364G+T366I+S388K+D470T+R483V;

V45L+T70R+A117S+L149R+S214Q+V258P+A276S+L281V+H358D+A364G+S388K+G394A+D470T+R483V;

V45L+A117S+L149R+S214Q+V258P+A276S+H358D+A364G+Q384A+S388K+D470T+L530R;

V45L+Q61K+A117S+L149R+S214Q+V258P+A276S+H358D+A364G+S388K+D470T+L530R;

V45L+T70R+A117S+L149R+S214Q+V258P+A276S+H358D+A364G+S388K+D470T+R483V;

V45L+A117S+L149R+S214Q+V258P+A276S+H358D+A364G+S388K+V393H+D470T+R483V;

V45L+A117S+L149R+S214Q+V258P+A276S+H358D+A364G+S388K+G394A+D470T+R483V;

V45L+A117S+L149R+S214Q+V258P+A276S+H358D+A364G+S388K+V393H+D470T+L530R;

V45L+A117S+L149R+S214Q+V258P+A276S+A286E+H358D+A364G+S388K+D470T+R483V;

D11E+V45L+A117S+L149R+S214Q+V258P+A276S+H358D+A364G+S388K+D470T+R483V;

V45L+T70R+A117S+L149R+S214Q+V258P+A276S+L281V+H358D+A364G+S388K+G394A+C468V+D470T+R483V;

V45L+A79C+A117S+L149R+S214Q+V258P+A276S+H358D+A364G+S388K+D470T V45L+A79C+L149R+S214E+V258P+A276S+A364G+S388K;

V45L+A117S+L149R+G187L+S214Q+V258P+A276S+H358D+A364G+S388K+D470T+L530R;

V45L+A117S+L149R+S214Q+V258P+A276S+H358D+A364G+Q384A+S388K+D470T+R483V;

V45L+A117S+L149R+G187K+S214Q+V258P+A276S+H358D+A364G+S388K+D470T+R483V;

V45L+A117S+L149R+S214Q+V258P+A276S+L281V+H358D+A364G+S388K+D470T+L530R;

V45L+A117S+L149R+S214E+V258P+A276S+H358D+A364G+S388K+D470T+R483V;

V45L+A117S+L149R+G187L+S214Q+V258P+A276S+H358D+A364G+S388K+D470T+R483V;

V45L+A117S+L149R+S214Q+V258P+A276S+H358D+A364G+S388K+G394Q+D470T+R483V;

V45L+A117S+L149R+S214Q+V258P+A276S+H358D+A364G+S388K+G394A+D470T+L530R;

V45L+A117S+L149R+S214Q+V258P+A276S+H358D+A364G+T366V+S388K+D470T+L530R;

V45L+A117S+L149R+S214Q+V258P+A276S+H358D+A364G+S388K+G394Q+D470T+L530R;

V45L+A117S+L149R+S214Q+V258P+A276S+S282N+H358D+A364G+S388K+D470T+R483V;

V45L+A117S+L149R+S214Q+V258P+A276S+H358D+A364G+S388K+N395D+D470T+R483V;

V45L+A117S+L149R+S214Q+V258P+A276S+H358D+A364G+S388K+D470T+N476D+R483V;

V45L+A117S+L149R+S214Q+V258P+A276S+V350L+H358D+A364G+S388K+D470T+R483V;

V45L+A117S+L149R+S214Q+V258P+A276S+H358D+A364G+S388K+D470T+T477K+R483V;

V45L+A117S+L149R+S214Q+V258P+A276S+H358D+A364G+S388K+D470T+R483V+Q496R;

V45L+T70R+A117S+L149R+S214Q+V258P+A276S+L281V+H358D+A364G+S388K+G394A+0468A+D470T+R483V;

V45L+A117S+L149R+S214Q+V258P+A276S+H358D+A364G+S388K+D470T+R483V+W495E;

V45L+A117S+L149R+S214Q+V258P+A276S+A286E+H358D+A364G+S388K+D470T+L530R;

V45L+A117S+L149R+G187K+S214Q+V258P+A276S+H358D+A364G+S388K+D470T+L530R;

V45L+A117S+L149R+S214Q+V258P+A276S+H358D+A364G+S388K+Y391V+D470T+R483V;

V45L+A117S+L149R+S214Q+V258P+A276S+H358D+A364G+S388K+D470T+R483V;

D9E+V45L+A117S+L149R+S214Q+V258P+A276S+H358D+A364G+S388K+D470T+L530R;

V45L+A117S+L149R+S214Q+V258P+A276S+H358D+A364G+S388K+D470T+T477V+R483V;

V45L+A117S+L149R+S214Q+V258P+A276S+H358D+A364G+S388K+D470T+N476N+R483V;

V45L+A117S+L149R+S214Q+V258P+A276S+H358D+A364G+S388K+V393R+D470T+R483V;

V45L+A117S+L149R+S214Q+V258P+A276S+H358D+A364G+S388K+D470T+T480L+L530R;

V45L+A117S+L149R+S214Q+V258P+A276S+H358D+A364G+S388K+D470T+S481A+L530R;

V45L+A117S+L149R+S214Q+W250Y+V258P+A276S+H358D+A364G+S388K+D470T+R483V;

V45L+A117S+L149R+S214Q+V258P+A276S+H358D+A364G+S388K+D470T+S481S+R483V;

V45L+A117S+L149R+S173Y+S214Q+V258P+A276S+H358D+A364G+S388K+D470T+R483V;

V45L+T70R+A117S+L149R+S214Q+C243V+V258P+A276S+L281V+H358D+A364G+S388K+G394A+D470T+R483V;

V45L+A117S+L149R+T180Q+S214Q+V258P+A276S+H358D+A364G+S388K+D470T+L530R;

D11E+V45L+A117S+L149R+S214Q+V258P+A276S+H358D+A364G+S388K+D470T+L530R;

V45L+A117S+L149R+S214E+V258P+A276S+H358D+A364G+S388K+D470T+L530R;

V45L+A117S+L149R+S214Q+V258P+A276S+H358D+A364G+S388K+D470T+N476D+L530R;

V45L+A117S+L149R+S214Q+V258P+A276S+A286S+H358D+A364G+S388K+D470T+R483V;

V45L+A117S+L149R+S214Q+V258P+A276S+H358D+A364G+S388K+D470T+R483V+Q496Q;

V45L+A117S+L149R+S214Q+V258P+A276Q+H358D+A364G+S388K+D470T+R483V;

V45L+A117S+L149R+S214Q+V258P+A276S+H358D+A364G+S388K+D470T+R483V+Q496E;

V45L+Q61R+A117S+L149R+S214Q+V258P+A276S+H358D+A364G+S388K+D470T+L530R;

V45L+A117S+L149R+S214Q+V258P+A276S+Q314A+H358D+A364G+S388K+D470T+L530R;

V45L+A117S+L149R+S214Q+V258P+A276Q+H358D+A364G+S388K+D470T+L530R;

V45L+A117S+L149R+S214Q+V258P+A276S+H358D+A364G+S388K+D470T+L478P+R483V;

A25W+V45L+A117S+L149R+S214Q+V258P+A276S+H358D+A364G+S388K+D470T+R483V;

V45L+A117S+L149R+T180Q+S214Q+V258P+A276S+H358D+A364G+S388K+D470T+R483V;

V45L+A117S+L149R+S214Q+V258P+A276S+H358D+A364G+S388K+D470T+R483V+L484K V45L+A117S+L149R+S214Q+V258P+A276S+H358D+A364G+S388K+D470T+N474G+L530R;

V45L+A117S+L149R+S214Q+V258P+A276S+H358D+A364G+S388K+N395D+D470T+L530R;

V45L+A117S+L149R+S214Q+V258P+A276S+H358D+A364G+T366V+S388K+D470T+R483V;

V45L+A117S+L149R+N168G+S214Q+V258P+A276S+H358D+A364G+S388K+D470T+R483V;

V45L+A117S+L149R+S214Q+V258P+A276S+H358D+A364G+S388K+D470T+R483V+L484S;

V45L+A117S+L149R+S214Q+V258P+A276S+H358D+A364G+S388K+D470T+L530R;

V45L+A117S+L149R+S214Q+V258P+A276S+H358D+A364G+S388K+D470T+R483V+L484D;

V45L+A117S+L149R+S214Q+V258P+A276S+H358D+A364G+S388K+D470T+R483V+S528H;

V45L+A117S+L149R+S214Q+V258P+A276S+H358D+A364G+S388K+D470T+R483V+S501N;

V45L+A117S+L149R+S173Y+S214Q+V258P+A276S+H358D+A364G+S388K+D470T+L530R;

V45L+A117S+L149R+S214Q+V258P+A276S+H358D+A364G+S388K+D470T+R483V+A499L;

V45L+S92D+A117S+L149R+S214Q+V258P+A276S+H358D+A364G+S388K+D470T+R483V;

V45L+A117S+L149R+S214Q+V258P+A276S+Q314A+H358D+A364G+S388K+D470T+R483V;

V45L+A79C+L149R+S214Q+V258P+A276S+A364G+S388K;

V45L+A117S+L149R+S214Q+V258P+A276S+N355G+H358D+A364G+S388K+D470T+L530R;

V45L+A117S+L149R+S214Q+V258P+A276S+V350L+H358D+A364G+S388K+D470T+L530R;

V45L+A117S+L149R+S214Q+V258P+A276S+H358D+A364G+S388K+D470T+S481S+L530R;

V45L+A117S+L149R+S214Q+V258P+A276S+H358D+A364G+S388K+D470T+Q496E+L530R;

V45L+A117S+L149R+N168G+S214Q+V258P+A276S+H358D+A364G+S388K+D470T+L530R;

V45L+A117S+L149R+S214Q+V258P+A276S+H358D+A364G+S388K+D470T+V498I+L530R;

V45L+A117S+L149R+S214Q+V258P+A276S+H358D+A364G+S388K+D470A+R483V;

V45N+A117S+L149R+S214Q+V258P+A276S+H358D+A364G+S388K+D470T+R483V;

V45L+A117S+L149R+S214Q+V258P+A276S+H358D+A364G+S388K+D470T+R483V+A499Y;

V45L+A79C+L149R+V258P+A276S+Q314A+A364G+S388K;

V45L+A79C+L149R+V258P+A276S+L281V+A364G+S388K;

V45L+A117S+L149R+S214Q+V258P+A276S+A286S+H358D+A364G+S388K+D470T+L530R;

V45L+A117S+L149R+S214Q+V258P+A276S+H358D+A364G+S388K+D470T+S501N+L530R;

V45L+A117S+L149R+S214Q+V258P+A276S+H358D+A364G+S388K+D470T+N476N+L530R;

V45L+A117S+L149R+S214Q+V258P+A276S+H358D+A364G+S388K+D470T+T477V+L530R;

V45L+A117S+L149R+S214Q+V258P+A276S+H358D+A364G+S388H+D470T+L530R;

V45L+A117S+L149R+S214Q+V258P+A276S+H358D+A364G+S388K+D470T+L478N+L530R;

V45L+S92D+A117S+L149R+S214Q+V258P+A276S+H358D+A364G+S388K+D470T+L530R;

V45L+A117S+L149R+S214Q+V258P+A276S+H358D+A364G+S388K+D470T+V500V+L530R;

V45L+A117S+L149R+S214Q+V258P+A276S+H358D+A364G+S388K+D470T+R483V+V500V;

V45L+A117S+L149R+S214Q+V258P+A276S+H358D+A364G+S388K+D470T+L478P+L530R;

V45L+A117S+L149R+S214Q+V258P+A276S+H358D+A364G+S388K+D470T+R483V+S501E;

V45L+A117S+L149R+S214Q+V258P+A276S+H358D+A364G+S388K+D470T+D525L+L530R;

V45L+A117S+L149R+S214Q+V258P+A276S+H358D+A364G+S388K+D470T+G526T+L530R;

V45L+A117S+L149R+S214Q+V258P+A276S+H358D+A364G+S388K+D470T+N527H+L530R;

V45L+A117S+L149R+S214Q+V258P+A276S+H358D+A364G+S388K+D470T+T477K+L530R;

M28P+V45L+A117S+L149R+S214Q+V258P+A276S+H358D+A364G+S388K+D470T+L530R;

D9E+V45L+A117S+L149R+S214Q+V258P+A276S+H358D+A364G+S388K+D470T+R483V;

V45L+A117S+L149R+S214Q+V258P+A276S+H358D+A364G+S388K+D470T+S501E+L530R;

V45L+A117S+L149R+S214Q+W250Y+V258P+A276S+H358D+A364G+S388K+D470T+L530R;

V45L+A117S+L149R+S214Q+V258P+A276S+H358D+A364G+S388K+D470T+A499L+L530R;

V45L+A117S+L149R+S214Q+V258P+A276S+H358D+A364G+S388K+D470T+A472I+L530R;

V45L+A117S+N127D+L149R+S214Q+V258P+A276S+H358D+A364G+S388K+D470T+R483V;

V45L+A117S+L149R+S214Q+V258P+A276S+H358D+A364G+S388K+D470T+V500T+L530R;

V45L+A117S+L149R+S214Q+V258P+A276S+H358D+A364G+S388K+D470T+R483V+N527H;

V45N+A117S+L149R+S214Q+V258P+A276S+H358D+A364G+S388K+D470T+L530R;

V45L+A117S+L149R+S214Q+V258P+A276S+H358D+A364G+S388K+D470T+T480N+L530R;

V45L+A117S+L149R+S214Q+V258P+A276S+H358D+A364G+S388K+D470T+R483V+V497P;

V45L+A117S+L149R+S214Q+V258P+A276S+H358D+A364G+S388K+D470T+R483V+V498I;

V45L+A79C+L149R+S173Y+V258P+A276S+A364G+S388K;

V45L+A117S+L149R+S214Q+V258P+A276S+H358D+A364G+S388K+D470T+T480N+R483V;

V45L+A117S+L149R+S214Q+H219V+V258P+A276S+H358D+A364G+S388K+D470T+L530R;

V45L+A117S+L149R+S214Q+V258P+A276S+H358D+A364G+S388A+D470T+L530R;

M28P+V45L+A117S+L149R+S214Q+V258P+A276S+H358D+A364G+S388K+D470T+R483V;

V45L+A117S+L149R+A170S+S214Q+V258P+A276S+H358D+A364G+S388K+D470T+R483V;

V45L+A79C+L149R+G187K+V258P+A276S+A364G+S388K;

V45L+A117S+L149R+S214Q+V258P+A276S+H358D+A364G+S388K+S389H+D470T+L530R;

V45L+D113T+A117S+L149R+S214Q+V258P+A276S+H358D+A364G+S388K+D470T+L530R;

V45L+A117S+L149R+N163H+S214Q+V258P+A276S+H358D+A364G+S388K+D470T+R483V;

V45L+A117S+L149R+S214Q+V258P+A276S+H358D+A364G+S388A+D470T+V45L+A117S+L149R+S214Q+V258P+A276S+H358D+A364G+S388L+D470T+R483V;

V45L+Q61R+A79C+L149R+V258P+A276S+A364G+S388K;

V45L+A79C+L149R+N168G+V258P+A276S+A364G+S388K;

V45L+A117S+L149R+T211H+S214Q+V258P+A276S+H358D+A364G+S388K+D470T+L530R;

V45L+A79C+L149R+T215V+V258P+A276S+A364G+S388K;

V45L+A117S+L149R+S214Q+V258P+A276S+H358D+A364G+S388K+D470T+R483V+V500T;

V45L+A79C+L149R+V258P+A276S+A286S+A364G+S388K;

V45L+A79C+L149R+H219V+V258P+A276S+A364G+S388K;

V45L+A79C+L149R+A170S+V258P+A276S+A364G+S388K;

V45L+D113T+A117S+L149R+S214Q+V258P+A276S+H358D+A364G+S388K+D470T+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A79C+L149R+V258P+A276S+A364G+S388K;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+T215I+V258P+A276S+H358D+A364G+S388K+D470T+R483V;

V45L+A117S+L149R+S214Q+H219I+V258P+A276S+H358D+A364G+S388K+D470T+R483V;

V45L+A117S+L149R+S214Q+V258P+A276S+H358D+A364G+S388K+D470T+L478N;

V45L+A117S+L149R+S214Q+V258P+A276S+L281V+H358D+A364G+S388K+D470T;

V45L+A117S+L149R+S214E+V258P+A276S+H358D+A364G+S388K+D470T;

V45L+T70R+A117S+L149R+S214Q+V258P+A276S+L281V+H358D+A364G+S388K+G394A+C468L+D470T+R483V;

V45L+A117S+L149R+S214Q+V258P+A276S+H358D+A364G+Q385C+S388K+D470T+L530R;

V45L+A117S+L149R+S214Q+V258P+A276S+H358D+A364G+T366I+S388K+D470T;

V45L+A117S+L149R+S214Q+V258P+A276S+H358D+A364G+S388K+D470T+S473G;

V45L+A117S+L149R+S214Q+V258P+A276S+H358D+A364G+S388K+D470T+L478P;

V45L+A117S+L149R+S214Q+V258P+A276S+H358D+A364G+S388K+G394D+D470T;

V45L+A117S+L149R+S214Q+V258P+A276S+A364G+Q384A+S388K+D470T+R483V;

D11E+V45L+A79C+L149R+V258P+A276S+A364G+S388K;

V45L+A117S+L149R+S214Q+V258P+A276S+H358D+A364G+S388K+D470T+R483V+L530R;

V45L+A117S+L149R+S214Q+V258P+A276S+H358D+A364G+S388K+D470T+N529T;

V45L+A117S+L149R+S214Q+V258P+A276S+H358D+A364G+S388K+G394Q+D470T;

V45L+A117S+L149R+G187L+S214Q+V258P+A276S+H358D+A364G+S388K+D470T;

V45L+A79C+L149R+V258P+A276S+A286E+A364G+S388K;

V45L+A117S+L149R+S214Q+V258P+A276S+H358D+A364G+S388K+D470T+R483V+G503L;

V45L+A117S+L149R+S214Q+V258P+A276S+Q314A+H358D+A364G+S388K+D470T;

V45L+A117S+L149R+S214Q+V258P+A276S+V350L+H358D+A364G+S388K+D470T;

V45L+A117S+L149R+G187K+S214Q+V258P+A276S+H358D+A364G+S388K+D470T;

V45L+A117S+L149R+S214Q+W250Y+V258P+A276S+H358D+A364G+S388K+D470T;

V45L+A117S+L149R+S214Q+V258P+A276S+A286E+H358D+A364G+S388K+D470T;

V45L+A117S+L149R+S214Q+V258P+A276S+H358D+A364G+Q384A+S388K+D470T;

V45L+A117S+L149R+S214Q+V258P+A276S+H358D+A364G+S388R+D470T;

V45L+A117S+L149R+S214Q+V258P+A276S+H358D+A364G+S388K+D470T+S528H;

V45L+A117S+L149R+S214Q+T215V+V258P+A276S+H358D+A364G+S388K+D470T+R483V;

V45L+A117S+L149R+S214Q+V258P+A276S+H358D+A364G+S388K+D470T+Q496R;

V45L+A117S+L149R+N163H+S214Q+V258P+A276S+H358D+A364G+S388K+D470T;

V45L+A117S+L149R+S214Q+V258P+A276S+S282N+H358D+A364G+S388K+D470T;

V45L+A117S+L149R+S214Q+V258P+A276Q+H358D+A364G+S388K+D470T;

D11E+V45L+A117S+L149R+S214Q+V258P+A276S+H358D+A364G+S388K+D470T;

V45L+A117S+L149R+S214Q+V258P+A276S+H358D+A364G+T366V+S388K+D470T;

V45L+A117S+L149R+S214Q+V258P+A276S+H358D+A364G+S388K+D470T+S473K;

V45L+A79C+L149R+T180Q+V258P+A276S+A364G+S388K;

V45L+A117S+L149R+S214Q+V258P+A276S+H358D+A364G+S388K+D470T+S481A+R483V;

V45L+A117S+L149R+S214Q+V258P+A276S+H358D+A364G+S388R+D470T+R483V;

V45L+A117S+L149R+S214Q+V258P+A276S+Q314A+A364G+S388K+D470T+R483V;

V45L+S92D+A117S+L149R+S214Q+V258P+A276S+H358D+A364G+S388K+D470T;

V45L+A117S+L149R+S214Q+V258P+N260H+A276S+H358D+A364G+S388K+D470T;

V45L+A117S+L149R+G187S+S214Q+V258P+A276S+H358D+A364G+S388K+D470T;

V45L+A117S+L149R+S214Q+V258P+A276S+H358D+A364G+S388K+D470T+T477K;

V45L+A117S+L149R+S214Q+V258P+A276S+H358D+A364G+S388K+D470T+N474G;

V45L+A117S+L149R+S214Q+V258P+A276S+H358D+A364G+S388K+N395D+D470T;

V45L+A117S+L149R+S214Q+V258P+A276S+H358D+A364G+S388K+D470T+S501N;

V45L+A117S+N127D+L149R+S214Q+V258P+A276S+H358D+A364G+S388K+D470T;

V45L+A117S+L149R+S214Q+V258P+A276S+H358D+A364G+S388K+V393R+D470T;

V45L+A117S+L149R+S173Y+S214Q+V258P+A276S+H358D+A364G+S388K+D470T;

V45L+A117S+L149R+S214Q+V258P+A276S+H358D+A364G+S388H+D470T;

V45L+A117S+L149R+S214Q+T215V+V258P+A276S+H358D+A364G+S388K+D470T+L530R;

V45L+A117S+L149R+G187S+S214Q+V258P+A276S+H358D+A364G+S388K+D470T+L530R;

V45L+A117S+L149R+T211H+S214Q+V258P+A276S+H358D+A364G+S388K+D470T+R483V;

V45L+A117S+L149R+S214Q+V258P+A276S+H358D+A364G+S388K+D470T+T480N;

V45L+A117S+L149R+N168G+S214Q+V258P+A276S+H358D+A364G+S388K+D470T;

V45L+A79C+L149R+H219I+V258P+A276S+A364G+S388K;

V45L+L149R+S214E+V258P+A276S+H358D+A364G+D470T;

V45L+Q61K+A117S+L149R+S214Q+V258P+A276S+A364G+S388K+D470T+R483V;

V45L+D113T+A117S+L149R+S214Q+V258P+A276S+H358D+A364G+S388K+D470T;

V45L+A117S+L149R+S214Q+V258P+A276S+A364G+T366V+S388K+D470T+R483V;

V45L+A117S+L149R+S214Q+V258P+A276S+H358T+A364G+S388K+D470T+R483V;

V45L+A117S+L149R+S214Q+V258P+A276S+A364G+S388K+V393R+D470T+R483V;

V45N+A79C+L149R+V258P+A276S+A364G+S388K;

V45L+A117S+L149R+S214Q+V258P+A276S+H358D+A364G+S388K+D470T+Q496Q;

V45L+A117S+L149R+S214Q+V258P+A276S+A364G+S388K+G394Q+D470T+R483V;

V45L+A79C+L149R+V258P+A276S+V350L+A364G+S388K;

V45L+L149R+S214E+V258P+A276S+H358D+A364G+D470T;

V45L+A117S+L149R+S214Q+V258P+A276S+A364G+T366I+S388K+D470T+R483V;

V45L+A117S+L149R+S214Q+V258P+A276S+H358D+A364G+S388K+D470T+R483V+G526T;

V45L+A117S+L149R+S214Q+V258P+A276S+H358D+A364G+S388K+D470T+L484S;

V45L+A79C+A117S+L149R+S214Q+V258P+A276S+H358D+A364G+S388K;

V45L+A117S+L149R+S214Q+V258P+A276S+H358D+A364G+S388K+D470T+A499L;

V45L+A117S+L149R+S214Q+V258P+A276S+H358T+A364G+S388K+D470T+L530R;

V45L+A79C+D113T+L149R+V258P+A276S+A364G+S388K;

V45L+A117S+L149R+S214Q+V258P+A276S+H358D+A364G+S388K+D470T+R483V+D525L;

V45L+A117S+L149R+S214Q+V258P+A276S+H358D+A364G+S388K+D470T+V498I;

V45L+A117S+L149R+S214Q+V258P+A276S+H358D+A364G+S388L+D470T;

V45L+T70R+A117S+L149R+S214Q+V258P+A276S+A364G+S388K+D470T+R483V;

V45L+A117S+L149R+S214Q+V258P+A276S+H358D+A364G+S388K+D470T+S528H+L530R;

V45L+A117S+L149R+S214Q+V258P+A276S+A364G+S388K+D470T+N476D+R483V;

D9E+V45L+A117S+L149R+S214Q+V258P+A276S+A364G+S388K+D470T+R483V;

V45L+A117S+L149R+S214Q+V258P+A276S+H358D+A364G+S388K+D470T+S501E;

V45L+A117S+L149R+S214Q+V258P+A276S+H358D+A364G+S388K+D470T+S481A;

V45L+A117S+L149R+S214Q+H219V+V258P+A276S+H358D+A364G+S388K+D470T;

V45L+A117S+L149R+S214Q+V258P+A276S+H358D+A364G+S388K+D470T+S481S;

V45L+A117S+L149R+G187K+S214Q+V258P+A276S+A364G+S388K+D470T+R483V;

V45L+A117S+L149R+S214Q+V258P+A276S+A364G+S388K+N395D+D470T+V45L+A117S+L149R+S214Q+V258P+A276S+H358D+A364G+S388K+I469T+D470T+R483V;

V45L+A79C+N127D+L149R+V258P+A276S+A364G+S388K;

D9E+V45L+A79C+L149R+V258P+A276S+A364G+S388K;

V45L+L149R+S214E+V258P+A276S+H358D+A364G+D470T;

V45L+A117S+L149R+G187L+S214Q+V258P+A276S+A364G+S388K+D470T+R483V;

V45L+A117S+L149R+S214Q+V258P+A276S+H358D+A364G+S388K+D470T+N529T+L530R;

V45L+A79C+L149R+W250Y+V258P+A276S+A364G+S388K;

V45L+A117S+L149R+S214Q+V258P+A276S+H358D+A364G+S388K+D470T+V497P;

V45L+A117S+L149R+S214Q+V258P+A276S+H358D+A364G+S388K+D470T+N527H;

V45L+A117S+L149R+S214Q+V258P+A276S+A364G+S388K+D470T+R483V+Q496Q;

V45L+A117S+L149R+N168G+S214Q+V258P+A276S+A364G+S388K+D470T+R483V;

V45L+A117S+L149R+S214Q+V258P+A276S+A364G+S388K+D470T+N476N+R483V;

V45L+A117S+L149R+S214Q+V258P+A276S+A364G+S388K+D470T+T477V+R483V;

V45L+A117S+L149R+S214Q+V258P+A276S+V350L+A364G+S388K+D470T+R483V;

V45L+A117S+L149R+S214Q+V258P+A276S+A364G+S388K+D470T+L478N+R483V;

V45L+A117S+L149R+S214E+V258P+A276S+A364G+S388K+D470T+R483V;

M28P+V45L+A117S+L149R+S214Q+V258P+A276S+H358D+A364G+S388K+D470T;

V45L+A117S+L149R+S214Q+V258P+A276S+A364G+S388K+V393H+D470T+R483V;

V45L+Q61R+A117S+L149R+S214Q+V258P+A276S+A364G+S388K+D470T+R483V;

V45L+A117S+L149R+S214Q+V258P+A276S+A364G+S388K+D470T+R483V+V497P;

V45L+A117S+L149R+S214Q+V258P+A276S+A364G+S388K+D470T+R483V+V498I;

V45L+A117S+L149R+S214Q+V258P+A276S+S282N+H358D+A364G+S388K+D470T+L530R;

V45L+T70R+A117S+L149R+S214Q+V258P+A276S+L281V+H358D+A364G+C375V+S388K+G394A+D470T+R483V;

V45L+A117S+L149R+S214Q+V258P+A276S+A286E+A364G+S388K+D470T+R483V;

V45L+A117S+L149R+S214Q+V258P+A276S+A364G+S388R+D470T+R483V;

V45L+A117S+L149R+S214Q+V258P+A276S+A364G+S388K+D470A+R483V;

V45L+A117S+L149R+S214Q+V258P+A276S+A364G+S388K+D470T+T477K+R483V;

V45L+A117S+L149R+S214Q+V258P+A276S+A364G+S388K+D470T+R483V+Q496E;

V45L+A117S+L149R+S214Q+V258P+A276S+A364G+S388K+D470T+R483V+Q496R;

V45L+A117S+L149R+S214Q+V258P+A276S+A364G+S388K+D470T+R483V+A499L;

V45L+L149R+S214E+V258P+A276S+H358D+A364G+D470T;

V45L+L149R+S214E+V258P+A276S+H358D+A364G+D470T;

V45L+A117S+L149R+S214Q+T215I+V258P+A276S+H358D+A364G+S388K+D470T;

V45L+T70R+A117S+L149R+S214Q+V258P+A276S+L281V+H358D+A364G+S388K+G394A+C468S+D470T+R483V;

V45L+A117S+L149R+S214Q+V258P+A276S+A364G+S388K+Y391V+D470T+R483V;

V45L+A117S+L149R+S214Q+V258P+A276Q+A364G+S388K+D470T+R483V;

V45L+A117S+L149R+S214Q+V258P+A276S+H358D+A364G+S388K+D470T+G503L+L530R;

V45L+A117S+L149R+S214Q+H219I+V258P+A276S+H358D+A364G+S388K+D470T+L530R;

V45L+A117S+L149R+S214Q+V258P+A276S+H358D+A364G+S388K+D470T+N476D;

V45L+A117S+L149R+S214Q+V258P+A276S+H358D+A364G+S388K+V393H+D470T;

V45L+A117S+L149R+S214Q+V258P+A276S+A364G+S388K+D470T+R483V;

V45L+A117S+L149R+S214Q+V258P+A276S+A286S+A364G+S388K+D470T+V45L+A117S+L149R+S214Q+V258P+A276S+A364G+S388K+D470T+R483V+S501E;

V45L+A117S+L149R+S214Q+V258P+A276S+A364G+S388K+D470T+R483V+N529T;

V45L+A117S+L149R+S214Q+V258P+A276S+A364G+S388K+D470T+R483V+S501N, V45L+A117S+L149R+S214Q+V258P+A276S+H358D+A364G+S388K+D470T;

V45L+A117S+L149R+S214Q+V258P+A276S+A364G+S388K+D470T+S481S+R483V;

V45L+A117S+L149R+S214Q+V258P+A276S+A364G+S388K+D470T+L478P+R483V;

D11E+V45L+A117S+L149R+S214Q+V258P+A276S+A364G+S388K+D470T+R483V;

V45L+A117S+L149R+S214Q+V258P+A276S+H358D+A364G+S388K+D470T+T477V;

V45L+A117S+L149R+S214Q+V258P+A276S+H358D+A364G+S388K+D470T+V497P+L530R;

V45L+A117S+L149R+T211H+S214Q+V258P+A276S+H358D+A364G+S388K+D470T;

V45L+A117S+L149R+S214Y+V258P+A276S+H358D+A364G+S388K+D470T+R483V;

V45L+A117S+L149R+S214Q+W250Y+V258P+A276S+A364G+S388K+D470T+R483V;

V45L+S92D+A117S+L149R+S214Q+V258P+A276S+A364G+S388K+D470T+R483V;

V45L+A117S+L149R+S214Q+V258P+A276S+A364G+S388K+D470T+R483V+N527H;

V45N+A117S+L149R+S214Q+V258P+A276S+A364G+S388K+D470T+R483V;

V45L+A117S+L149R+S214Q+V258P+A276S+H358D+A364G+Q385C+S388K+D470T;

V45L+A117S+L149R+S173Y+S214Q+V258P+A276S+A364G+S388K+D470T+R483V;

V45L+A117S+L149R+S214Q+V258P+A276S+A364G+S388H+D470T+R483V;

V45L+A117S+L149R+S214Q+V258P+A276S+A364G+S388K+S389H+D470T+R483V;

V45L+A117S+L149R+S214Q+H219V+V258P+A276S+A364G+S388K+D470T+V45L+A79C+A117S+L149R+S214Q+V258P+A276S+A364G+S388K+D470T;

V45L+A117S+L149R+S214Q+T215I+V258P+A276S+H358D+A364G+S388K+D470T+L530R;

V45L+A117S+L149R+S214Q+V258P+A276S+H358D+A364G+S388K+D470T+Q496E;

V45L+A117S+L149R+A170S+S214Q+V258P+A276S+H358D+A364G+S388K+D470T;

V45L+A117S+L149R+S214Q+V258P+A276S+H358D+A364G+S388K+S389V+D470T+R483V;

V45L+A117S+L149R+S214Q+V258P+A276S+H358D+A364G+S388K+S389V+D470T+L530R;

V45L+A117S+L149R+S214Q+V258P+A276S+H358D+A364G+S388A+D470T;

V45L+A117S+L149R+S214Q+V258P+A276S+H358D+A364G+S388K+S389V+D470T;

V45L+T70R+A117S+L149R+S214Q+V258P+A276S+H358D+A364G+S388K+D470T;

V45L+A117S+L149R+S214Q+H219I+V258P+A276S+A364G+S388K+D470T+R483V;

V45L+A79C+L149R+N163H+V258P+A276S+A364G+S388K;

V45L+A117S+L149R+S214Q+V258P+A276S+A364G+S388K+D470T+R483V+A499Y;

V45L+A117S+L149R+S214Q+V258P+A276S+L281V+A364G+S388K+D470T+R483V;

V45L+A117S+L149R+S214Q+V258P+A276S+A364G+S388K+D470T+R483V+V500T;

V45L+A117S+L149R+T180Q+S214Q+V258P+A276S+H358D+A364G+S388K+D470T;

V45L+A117S+L149R+S214Q+V258P+A276S+H358D+A364G+S388K+D470T+V500V;

V45L+A117S+L149R+S214Q+V258P+A276S+H358D+A364G+S388K+D470T+G526T;

V45L+C60L+T70R+A117S+L149R+S214Q+V258P+A276S+L281V+H358D+A364G+S388K+G394A+D470T+R483V;

V45L+C69L+T70R+A117S+L149R+S214Q+V258P+A276S+L281V+H358D+A364G+S388K+G394A+D470T+R483V;

V45L+A117S+L149R+S214Q+V258P+A276S+A364G+S388A+D470T+R483V;

V45L+A117S+L149R+S214Q+V258P+A276S+A286S+H358D+A364G+S388K+D470T;

V45L+A117S+L149R+S214Q+V258P+A276S+H358D+A364G+S388K+D470T+R483V+V497D;

V45L+A117S+L149R+S214Q+V258P+A276S+H358D+A364G+S388K+D470A+L530R;

V45L+A117S+L149R+S214Q+V258P+A276S+A364G+S388K+D470T+R483V+D525L;

V45L+A117S+L149R+S214Q+V258P+A276S+H358D+A364G+S388K+D470T+N476N;

V45L+A117S+L149R+S214Q+V258P+A276S+L281V+A364G+S388K+D470T;

V45L+A117S+L149R+S214Q+V258P+A276S+A364G+Q384A+S388K+D470T;

V45L+A117S+L149R+S214Q+V258P+A276S+A286E+A364G+S388K+D470T;

V45L+A117S+L149R+S214Q+V258P+A276S+A364G+S388K+D470T+T477K;

V45L+A117S+L149R+S214Q+V258P+A276S+H358D+A364G+S388K+S389H+D470T;

V45L+A117S+L149R+S214Y+V258P+A276S+H358D+A364G+S388K+D470T+L530R;

V45L+A117S+L149R+S214Y+V258P+A276S+H358D+A364G+S388K+D470T;

V45L+A117S+L149R+S214Q+V258P+A276S+A364G+S388K+G394A+D470T;

V45L+A117S+L149R+S214Q+V258P+A276S+A364G+S388K+D470T+N476D;

V45L+A117S+L149R+S214Q+V258P+A276S+H358D+A364G+S388K+D470T+L478N+R483V;

V45L+C60A+T70R+A117S+L149R+S214Q+V258P+A276S+L281V+H358D+A364G+S388K+G394A+D470T+R483V;

V45L+A117S+L149R+S214Q+V258P+A276S+A364G+S388K+V393H+D470T;

V45L+A117S+L149R+S214Q+V258P+A276S+A364G+S388K+N395D+D470T;

A25W+V45L+A117S+L149R+S214Q+V258P+A276S+H358D+A364G+S388K+D470T;

V45L+T70R+A117S+L149R+S214Q+C243L+V258P+A276S+L281V+H358D+A364G+S388K+G394A+D470T+R483V;

V45L+A117S+L149R+S214Q+V258P+A276S+H358T+A364G+S388K+D470T;

V45L+A117S+L149R+S214Q+V258P+A276S+A364G+S388K+D470T+L478N;

V45L+C60S+T70R+A117S+L149R+S214Q+V258P+A276S+L281V+H358D+A364G+S388K+G394A+D470T+R483V;

V45L+A117S+L149R+S214Q+W250Y+V258P+A276S+A364G+S388K+D470T;

V45L+A117S+L149R+S214Q+T215V+V258P+A276S+A364G+S388K+D470T+R483V;

V45L+Q61R+A117S+L149R+S214Q+V258P+A276S+H358D+A364G+S388K+D470T;

V45L+A117S+L149R+G187L+S214Q+V258P+A276S+A364G+S388K+D470T;

D9E+V45L+A117S+L149R+S214Q+V258P+A276S+A364G+S388K+D470T;

V45L+A117S+L149R+S214Q+V258P+A276S+H358D+A364G;

V45L+C69A+T70R+A117S+L149R+S214Q+V258P+A276S+L281V+H358D+A364G+S388K+G394A+D470T+R483V;

V45L+T70R+A117S+L149R+C161S+S214Q+V258P+A276S+L281V+H358D+A364G+S388K+G394A+D470T+R483V;

V45L+A117S+L149R+S214Q+H219I+V258P+A276S+H358D+A364G+S388K+D470T;

V45L+A117S+L149R+S214Q+V258P+A276S+S282N+A364G+S388K+D470T;

V45L+A117S+L149R+T211H+S214Q+V258P+A276S+A364G+S388K+D470T+R483V;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

D11E+V45L+A117S+L149R+S214Q+V258P+A276S+A364G+S388K+D470T;

V45L+A117S+L149R+S214Q+V258P+A276S+A364G+S388K+D470T+N476N;

V45L+A117S+L149R+S214Q+V258P+A276S+A364G+S388K+G394D+D470T;

V45L+Q61K+A117S+L149R+S214Q+V258P+A276S+A364G+S388K+D470T;

V45L+A117S+L149R+S214Q+V258P+A276S+H358D+A364G+S388K+D470T+G503L;

V45L+A117S+L149R+S214Q+V258P+A276S+A286S+A364G+S388K+D470T;

V45L+A117S+L149R+S214Q+V258P+A276S+H358D+A364G+S388K+D470A;

V45L+A117S+L149R+S214Q+V258P+A276S+A364G+S388K+G394Q+D470T;

V45L+A117S+L149R+S214Q+V258P+A276S+A364G+S388R+D470T;

V45L+A117S+L149R+S214Q+V258P+A276S+A364G+S388K+D470T+L478P;

V45L+A117S+L149R+S214Q+V258P+A276S+V350L+A364G+S388K+D470T;

V45L+D113T+A117S+L149R+S214Q+V258P+A276S+A364G+S388K+D470T;

V45L+A117S+L149R+T180Q+S214Q+V258P+A276S+A364G+S388K+D470T;

V45L+C69V+T70R+A117S+L149R+S214Q+V258P+A276S+L281V+H358D+A364G+S388K+G394A+D470T+R483V;

V45L+A117S+L149R+S214Q+V258P+A276S+H358D+A364G+S388K+D470T+D525L;

V45L+A117S+L149R+S214Q+V258P+A276S+A364G+S388K+D470T;

V45L+A117S+L149R+S214Q+V258P+A276S+H358D+A364G+S388K+D470T+V500T;

V45L+A117S+L149R+S214Q+V258P+A276S+A364G+S388K+S389H+D470T;

V45L+A117S+L149R+S214Q+V258P+A276S+A364G+S388K+V393R+D470T;

V45L+A117S+L149R+S214Q+V258P+A276S+A364G+S388K+D470T+Q496R;

V45L+A117S+N127D+L149R+S214Q+V258P+A276S+A364G+S388K+D470T;

V45L+A117S+L149R+S214Q+V258P+A276S+A364G+S388K+D470T+A499Y;

V45L+A117S+L149R+S214Q+T215V+V258P+A276S+H358D+A364G+S388K+D470T;

V45L+A117S+L149R+S214Q+V258P+A276S+H358D+A364G+S388K+D470T+T480L;

V45L+A117S+L149R+S214Q+V258P+A276S+A364G+S388L+D470T;

V45L+A117S+L149R+S173Y+S214Q+V258P+A276S+A364G+S388K+D470T;

V45L+S92D+A117S+L149R+S214Q+V258P+A276S+A364G+S388K+D470T;

V45L+A117S+L149R+S214Q+V258P+A276S+A364G+S388K+S389V+D470T+R483V;

V45L+A117S+L149R+S214Q+V258P+A276S+A364G+S388K+D470T+N529T;

V45L+C60V+T70R+A117S+L149R+S214Q+V258P+A276S+L281V+H358D+A364G+S388K+G394A+D470T+R483V;

V45L+A117S+L149R+S214Q+V258P+A276S+A364G+S388K+D470T+R483V+V497E;

V45L+A117S+L149R+S214Q+V258P+A276S+H358D+A364G+S388K+L478N;

V45L+T70R+A117S+L149R+S214Q+V258P+A276S+A364G+S388K+D470T;

M28P+V45L+A117S+L149R+S214Q+V258P+A276S+A364G+S388K+D470T;

V45L+A117S+L149R+S214Q+V258P+A276Q+A364G+S388K+D470T;

V45L+A117S+L149R+S214Q+V258P+A276S+H358D+A364G+S388K+D470V+R483V;

V45L+A117S+L149R+S214Y+V258P+A276S+A364G+S388K+D470T+R483V;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

V45L+Q61R+A117S+L149R+S214Q+V258P+A276S+A364G+S388K+D470T;

V45L+A117S+L149R+S214Q+T215I+V258P+A276S+A364G+S388K+D470T;

V45L+A117S+L149R+S214Q+V258P+A276S+A364G+S388K+D470T+Q496E;

V45L+A117S+L149R+G187K+S214Q+V258P+A276S+A364G+S388K+D470T;

V45L+A117S+L149R+S214Q+V258P+A276S+A364G+S388K+D470T+W495I;

V45L+A117S+L149R+S214Q+V258P+A276S+A364G+S388K+D470T+V498I;

V45L+A117S+L149R+S214Q+V258P+A276S+H358D+A364G+S388K+D470K+R483V;

V45L+A117S+L149R+S214Q+V258P+A276S+A364G+S388K+D470T+V500V;

D11E+V45L+A117S+L149R+S214Q+V258P+A276S+H358D+A364G+S388K;

V45L+A117S+L149R+A170S+S214Q+V258P+A276S+A364G+S388K+D470T;

V45L+A117S+L149R+S214Q+V258P+A276S+A364G+S388K+Y391V+D470T;

V45L+A117S+L149R+S214Q+V258P+A276S+Q314A+A364G+S388K+D470T;

V45L+A117S+L149R+S214Q+V258P+A276S+A364G+S388K+D470T+T477V;

A25W+V45L+A79C+L149R+S214E+V258P+A276S+A364G;

V45L+A117S+L149R+S214Q+V258P+A276S+H358D+A364G+S388K+G394Q;

V45L+A117S+L149R+S214Q+H219V+V258P+A276S+A364G+S388K+D470T;

V45L+A117S+L149R+S214Q+V258P+A276S+A364G+S388K+D470T+Q496Q;

V45L+T70R+A117S+L149R+S214Q+V258P+A276S+L281V+H358D+A364G+C375A+S388K+G394A+D470T+R483V;

V45L+A117S+L149R+D192E+S214Q+V258P+A276S+H358D+A364G;

V45L+A117S+L149R+S214Q+V258P+A276S+H358D+A364G+S388K+D470T+A472L;

V45L+A117S+L149R+S214Q+V258P+A276S+H358D+A364G+S388K+A499Y;

V45L+A117S+L149R+S214Q+T215V+V258P+A276S+A364G+S388K+D470T;

V45L+A117S+L149R+S214Q+V258P+A276S+A364G+Q385C+S388K+D470T;

V45L+A117S+L149R+S214Q+V258P+A276S+A364G+S388A+D470T;

V45L+A117S+L149R+S214Q+V258P+A276S+A364G+S388H+D470T;

V45L+A117S+L149R+S214Q+V258P+A276S+A364G+S388K+D470T+T480N;

V45L+A117S+L149R+S214Q+V258P+A276S+A364G+S388K+D470T+V497P;

V45L+A117S+L149R+S214Q+V258P+A276S+A364G+S388K+D470T+A499L;

V45L+A117S+L149R+S214Q+V258P+A276S+A364G+S388K+D470T+S501N;

V45L+A117S+L149R+S214Q+V258P+A276S+A364G+D470T;

V45L+A117S+L149R+S214Q+V258P+A276S+H358D+A364G+Q384A+S388K;

V45L+T70R+A117S+L149R+S214Q+V258P+A276S+L281V+H358D+A364G+C375S+S388K+G394A+D470T+R483V;

V45L+A117S+L149R+S214Q+V258P+A276S+A364G+S388L+D470T+R483V;

V45L+A79C+S92D+L149R+V258P+A276S+A364G+S388K;

V45L+T70R+A117S+L149R+S214Q+V258P+A276S+L281V+H358D+A364G+S388K+G394A+D470T+R483V+C540A;

V45N+A117S+L149R+S214Q+V258P+A276S+A364G+S388K+D470T;

V45L+A117S+L149R+S214Q+V258P+A276S+H358D+A364G+S388K+G394A;

V45L+C69S+T70R+A117S+L149R+S214Q+V258P+A276S+L281V+H358D+A364G+S388K+G394A+D470T+R483V;

V45L+A117S+L149R+S214Q+V258P+A276S+H358D+A364G+S388K+V498I;

V45L+A117S+L149R+S214Q+V258P+A276S+H358D+A364G;

V45L+A117S+L149R+S214Q+H219I+V258P+A276S+A364G+S388K+D470T;

V45L+A117S+L149R+N163H+S214Q+V258P+A276S+A364G+S388K+D470T;

V45L+L149R+S214Q+V258P+A276S+H358D+A364G;

V45L+A117S+L149R+S214Q+V258P+A276S+Q314A+H358D+A364G+S388K;

V45L+A117S+L149R+S173Y+S214Q+V258P+A276S+H358D+A364G+S388K;

V45L+A117S+L149R+S214E+V258P+A276S+H358D+A364G+S388K;

V45L+A117S+L149R+S214Q+V258P+A276S+H358D+A364G+S388K+G394D;

A25W+V45L+A79C+L149R+V258P+A276S+A364G;

V45L+L149R+S214Q+V258P+A276S+A286E+A364G+S388K;

V45L+Q61R+A117S+L149R+S214Q+V258P+A276S+H358D+A364G+S388K;

V45L+A117S+L149R+S214Q+V258P+A276S+H358D+A364G+T366V+S388K;

V45L+A117S+L149R+D192E+S214Q+V258P+A276S+H358D+A364G;

V45L+A117S+L149R+T180Q+S214Q+V258P+A276S+H358D+A364G+S388K;

V45L+A117S+L149R+S214Q+T215I+V258P+A276S+H358D+A364G+S388K;

V45L+A117S+L149R+S214Q+W250Y+V258P+A276S+H358D+A364G+S388K;

V45L+L149R+S214Q+T215I+V258P+A276S+A364G+S388K;

V45L+A117S+L149R+S214Q+V258P+A276S+H358D+A364G+S388K+N476D;

V45L+D113T+A117S+L149R+S214Q+V258P+A276S+A364G+S388K+D470T+R483V;

V45L+A117S+L149R+S214Q+T215K+V258P+A276S+A364G+S388K+D470T+R483V;

V45L+A117S+L149R+S214Q+V258P+A276S+H358D+A364G+S388K;

V45L+A117S+L149R+S214Q+V258P+A276S+H358D+A364G+S388K+T477V;

V45L+A117S+L149R+S214Q+V258P+A276S+H358D+A364G+S388K+L478P;

V45L+A117S+L149R+S214Q+V258P+A276S+H358D+A364G+S388K+S481A;

A25W+V45L+L149R+S214Q+V258P+A276S+A364G+S388K;

V45L+A117S+L149R+T211H+S214Q+V258P+A276S+A364G+S388K+D470T;

V45L+A117S+L149R+S214Q+T215K+V258P+A276S+H358D+A364G+S388K+D470T+R483V;

V45L+A79C+L149R+V258P+A276S+L281V+A364G;

V45L+A117S+L149R+S214Q+V258P+A276S+A364G+S388K+R483V;

V45L+A117S+L149R+S214Q+V258P+A276S+H358D+A364G+S388K+V500T;

V45L+A117S+L149R+S214Q+V258P+A276S+V350L+H358D+A364G+S388K;

V45L+A117S+L149R+G187K+S214Q+V258P+A276S+H358D+A364G+S388K;

V45L+T70R+A117S+L149R+S214Q+V258P+A276S+L281V+H358D+A364G+C375L+S388K+G394A+D470T+R483V;

V45L+L149R+A170S+S214Q+V258P+A276S+A364G+S388K;

V45L+A117S+L149R+S214E+V258P+A276S+A364G+S388K+D470T;

V45L+A117S+L149R+S214Q+V258P+A276S+A364G+S388K+D470V+R483V;

V45L+A117S+L149R+S214Q+V258P+A276S+H358D+A364G+S388K+V500V;

V45L+Q61K+L149R+S214Q+V258P+A276S+A364G+S388K;

V45L+L149R+N168G+S214Q+V258P+A276S+A364G+S388K;

V45L+A117S+L149R+N163H+S214Q+V258P+A276S+H358D+A364G+S388K;

V45L+A79C+D95D+L149R+V258P+A276S+A364G+S388K;

V45L+Q77E+A79C+L149R+V258P+A276S+A364G+S388K;

V45L+A117S+L149R+S214Q+V258P+A276S+A364G+S388K+D470T+R483V+V497W;

V45L+L149R+S214Q+V258P+A276S+S282N+A364G+S388K;

V45L+A117S+L149R+S214Q+V258P+A276S+H358D+A364G+S388R;

V45L+A117S+L149R+S214Q+V258P+A276S+H358D+A364G+S388K+S481S;

V45L+A117S+L149R+S214Q+V258P+A276S+A364G+S388K+D470T+V500T;

V45L+T70R+A117S+L149R+S214Q+V258P+A276S+L281V+H358D+A364G+S388K+G394A+D470T+R483V+C540L;

V45L+T70R+A117S+L149R+S214Q+V258P+A276S+L281V+H358D+A364G+S388K+G394A+D470T+R483V+C522S;

V45L+A117S+L149R+S214Q+V258P+A276S+H358D+A364G+S388K+W495S;

V45L+L149R+S214Q+V258P+A276S+Q314A+A364G+S388K;

V45L+D113T+A117S+L149R+S214Q+V258P+A276S+H358D+A364G+S388K;

V45L+A117S+L149R+S214Q+V258P+A276S+H358D+A364G+S388K+D470T+R483V+V497W;

V45L+T70R+A117S+L149R+S214Q+V258P+A276S+L281V+H358D+A364G+S388K+G394A+D470T+R483V+C540S;

V45L+T70R+A117S+L149R+S214Q+V258P+A276S+L281V+H358D+A364G+S388K+G394A+C396S+D470T+R483V;

V45L+T70R+L149R+S214Q+V258P+A276S+A364G+S388K;

V45L+A117S+L149R+S214Q+V258P+A276S+H358D+A364G+S388K+N474G;

V45L+A117S+L149R+S214Q+V258P+A276S+S282N+H358D+A364G+S388K;

V45L+A117S+L149R+S214Q+V258P+A276S+H358D+A364G+S388K+Q496Q;

V45L+A117S+L149R+S214Q+V258P+A276S+A364G+S388K+S389V+D470T;

V45L+T70R+A117S+L149R+S214Q+V258P+A276S+L281V+H358D+A364G+S388K+G394A+C396A+D470T+R483V;

V45L+A117S+L149R+S214Q+V258P+A276S+H358D+A364G+S388K+S501E;

A25W+V45L+L149R+S214E+V258P+A276S+A364G;

V45L+L149R+S214Q+W250Y+V258P+A276S+A364G+S388K;

V45L+L149R+G187K+S214Q+V258P+A276S+A364G+S388K;

V45L+A117S+L149R+S214Q+V258P+A276S+H358D+A364G+S388K+N476N;

V45L+A117S+L149R+S214Q+V258P+A276S+H358D+A364G+S388K+S473K;

V45L+A117S+L149R+G187L+S214Q+V258P+A276S+H358D+A364G+S388K;

C40L+V45L+T70R+A117S+L149R+S214Q+V258P+A276S+L281V+H358D+A364G+S388K+G394A+D470T+R483V;

V45L+A117S+L149R+S214Q+V258P+A276S+H358D+A364G+S388K+Q496E;

A25W+V45L+A117S+L149R+S214Q+V258P+A276S+H358D+A364G+S388K;

V45L+L149R+V258P+A276S+L281V+H358T+A364G+S388K;

V45L+A117S+L149R+S214Q+V258P+A276S+H358D+A364G+S388A;

V45L+A117S+L149R+N168G+S214Q+V258P+A276S+H358D+A364G+S388K;

V45L+A117S+L149R+S214Q+V258P+A276S+H358D+A364G+S388K+S389H;

M28P+V45L+L149R+S214Q+V258P+A276S+A364G+S388K;

V45L+A117S+L149R+S214Q+V258P+A276S+A364G+S388K+D470T+T480L;

V45L+A117S+L149R+S214Q+V258P+A276S+A364G+Q384A+S388K;

V45L+A93S+L149R+A252S+H334D;

C40A+V45L+T70R+A117S+L149R+S214Q+V258P+A276S+L281V+H358D+A364G+S388K+G394A+D470T+R483V;

V45L+L149R+G187L+S214Q+V258P+A276S+A364G+S388K;

V45L+A117S+L149R+S214Q+V258P+A276S+H358D+A364G+Q385C+S388K;

V45L+L149R+S214Q+V258P+A276S+A286S+A364G+S388K;

V45L+S92D+A117S+L149R+S214Q+V258P+A276S+H358D+A364G+S388K;

V45L+A117S+L149R+S214Q+V258P+A276S+H358D+A364G+S388L;

V45L+A117S+L149R+S214Q+V258P+A276S+A286E+A364G+S388K;

D9E+V45L+L149R+S214Q+V258P+A276S+A364G+S388K;

V45L+L149R+S173Y+S214Q+V258P+A276S+A364G+S388K;

V45L+A117S+L149R+A170S+S214Q+V258P+A276S+H358D+A364G+S388K;

V45L+A117S+L149R+S214Q+V258P+A276S+H358D+A364G+S388K+Y391V;

V45L+A117S+L149R+S214Q+V258P+A276S+L281V+A364G+S388K;

V45L+A117S+L149R+S214Q+V258P+A276S+A364G+S388K+G394D;

V45L+A117S+L149R+S214Q+V258P+A276S+A364G+S388K+S473G;

V45L+A79C+L149R+V258P+A276S+H358T+A364G+S388K;

V45L+L149R+S214Q+V258P+A276Q+A364G+S388K;

A25W+V45L+L149R+S214E+V258P+A276S+H358T+A364G;

V45L+A117S+L149R+S214Q+V258P+A276S+H358D+A364G+S388K+T480N;

D9E+V45L+A117S+L149R+S214Q+V258P+A276S+H358D+A364G+S388K;

V45L+A117S+L149R+S214Q+H219I+V258P+A276S+H358D+A364G+S388K;

C40S+V45L+T70R+A117S+L149R+S214Q+V258P+A276S+L281V+H358D+A364G+S388K+G394A+D470T+R483V;

C40V+V45L+T70R+A117S+L149R+S214Q+V258P+A276S+L281V+H358D+A364G+S388K+G394A+D470T+R483V;

A25W+V45L+L149R+S214E+V258P+A276S+A286E+A364G;

V45L+A117S+L149R+S214Q+V258P+A276S+A364G+S388K+G394A;

V45L+A117S+L149R+S214Q+V258P+A276S+H358D+A364G+S388K+A499L;

A25W+V45L+Q61K+L149R+S214E+V258P+A276S+A364G;

V45L+A117S+L149R+S214Q+T215V+V258P+A276S+H358D+A364G+S388K;

A25W+V45L+L149R+V258P+A276S+H358T+A364G+S388K;

V45L+A117S+L149R+S214Q+V258P+A276S+A364G+S388K+D470T+R483V+W495E;

A25W+V45L+A79C+L149R+V258P+A276S;

V45L+L149R+S214Q+V258P+A276S+V350L+A364G+S388K;

D11E+V45L+L149R+S214Q+V258P+A276S+A364G+S388K;

V45L+L149R+H219I+V258P+A276S+H358T+A364G+S388K;

V45L+T70R+A117S+L149R+S214Q+V258P+A276S+L281V+H358D+A364G+S388K+G394A+C396V+D470T+R483V;

V45N+L149R+S214Q+V258P+A276S+A364G+S388K;

V45L+A117S+L149R+S214Q+H219V+V258P+A276S+H358D+A364G+S388K;

V45N+A117S+L149R+S214Q+V258P+A276S+H358D+A364G+S388K;

V45L+S92D+L149R+S214Q+V258P+A276S+A364G+S388K;

V45L+L149R+V258P+A276S+H358T+A364G;

V45L+A117S+L149R+S214Q+V258P+A276S+A364G+S388K+L478P;

V45L+A117S+L149R+S214Q+V258P+A276S+A364G+S388K+S481A;

V45L+A117S+L149R+S214Q+V258P+A276S+Q314A+A364G+S388K;

A25W+V45L+L149R+T211H+S214E+V258P+A276S+A364G;

A25W+V45L+L149R+S214E+H219V+V258P+A276S+A364G;

V45L+A117S+L149R+S214Q+V258P+A276S+H358D+A364G+S388K+S389V;

V45L+A117S+L149R+S214Q+V258P+A276S+H358D+A364G+S388K+V393H;

V45L+A117S+L149R+S214Q+V258P+A276Q+H358D+A364G+S388K;

V45L+A117S+N127D+L149R+S214Q+V258P+A276S+H358D+A364G+S388K;

V45L+L149R+W250Y+V258P+A276S+H358T+A364G+S388K;

D9E+V45L+L149R+V258P+A276S+H358T+A364G+S388K;

V45L+L149R+H219V+V258P+A276S+H358T+A364G+S388K;

V45L+A117S+L149R+S214Q+V258P+A276S+H358D+A364G+S388K+A472L;

A25W+V45L+L149R+V258P+A276S+A364G;

V45L+A117S+L149R+G187K+S214Q+V258P+A276S+A364G+S388K;

V45L+A117S+L149R+S214Q+V258P+A276S+A364G+S388K+L478N;

V45L+A117S+L149R+S214Q+V258P+A276S+A364G+S388K+V393R;

V45L+A117S+L149R+S214E+V258P+A276S+A364G+S388K;

V45L+A117S+L149R+S214Q+V258P+A276S+A364G+T366V+S388K;

V45L+A117S+L149R+S214Q+V258P+A276S+H358T+A364G+S388K;

D11E+A25W+V45L+L149R+S214E+V258P+A276S+A364G;

V45L+T70R+A117S+L149R+C161V+S214Q+V258P+A276S+L281V+H358D+A364G+S388K+G394A+D470T+R483V;

V45L+A117S+L149R+T211H+S214Q+V258P+A276S+H358D+A364G+S388K;

V45L+L149R+V258P+A276S+Q314A+H358T+A364G+S388K;

D11E+V45L+A117S+L149R+S214Q+V258P+A276S+A364G+S388K;

V45L+A117S+L149R+S214Q+V258P+A276S+A364G+S388K+G394Q;

V45L+A117S+L149R+S214Q+V258P+A276S+A364G+S388K+N395D;

V45L+A117S+L149R+S214Q+V258P+A276S+A364G+S388K+V393H;

V45L+A117S+L149R+S214Q+V258P+A276S+A364G+S388K+S501E;

V45L+A117S+L149R+S214Q+V258P+A276S+A364G+S388K+S528H;

V45L+T70R+A117S+L149R+S214Q+V258P+A276S+H358D+A364G+S388K+D470T+L530R;

V45L+A117S+L149R+S214Q+V258P+A276S+A364G+S388L;

V45L+T70R+A117S+L149R+S214Q+V258P+A276S+L281V+H358D+A364G+S388K+G394A+C396L+D470T+R483V;

V45L+L149R+T180Q+S214Q+V258P+A276S+A364G+S388K;

V45L+D113T+L149R+S214Q+V258P+A276S+A364G+S388K;

V45L+A117S+L149R+S214Q+V258P+A276S+H358D+A364G+S388K+T480L;

V45L+A93S+L149R+A252S+H334D;

V45L+A93S+L149R+V234P+A252S;

V45L+A117S+L149R+S214Q+V258P+A276S+H358D+A364G+S388H;

A25W+V45L+Q61R+L149R+S214E+V258P+A276S+A364G;

V45L+A117S+L149R+S214Q+T215K+V258P+A276S+H358D+A364G+S388K+V45L+A117S+L149R+S214Q+V258P+A276S+A364G+S388K+S389H;

V45L+A117S+L149R+S214Q+V258P+A276S+A364G+T366I+S388K;

V45L+A117S+L149R+S214Q+V258P+A276S+A364G+S388K+N474G;

A25W+V45L+L149R+S214E+V258P+A276S+L281V+A364G;

D9E+A25W+V45L+L149R+S214E+V258P+A276S+A364G;

V45L+A117S+L149R+S214Q+T215K+V258P+A276S+H358D+A364G+S388K+D470T+L530R;

V45L+A117S+L149R+S214Y+V258P+A276S+H358D+A364G+S388K;

V45L+A117S+L149R+S214Q+V258P+A276S+H358D+A364G+S388K+V393R;

V45L+Q61R+L149R+V258P+A276S+H358T+A364G+S388K;

V45L+L149R+S214Q+V258P+A276S+A364G+S388K;

V45L+D113T+L149R+V258P+A276S+H358T+A364G+S388K;

V45L+A117S+L149R+S214Q+V258P+A276S+A286S+A364G+S388K;

V45L+A117S+L149R+S214Q+V258P+A276S+A364G+S388R;

V45L+A117S+L149R+S214Q+V258P+A276S+A364G+S388K+A499Y;

V45L+A117S+L149R+S214Q+V258P+A276S+A364G+S388K+V500V;

V45L+Q61R+L149R+S214Q+V258P+A276S+A364G+S388K;

V45L+L149R+S214E+V258P+A276S+H358T+A364G;

V45L+A117S+L149R+S214Q+V258P+A276Q+A364G+S388K;

V45L+L149R+V258P+A276S+L281V+A364G;

V45L+A117S+L149R+S214Q+W250Y+V258P+A276S+A364G+S388K;

V45L+A117S+L149R+S214Q+V258P+A276S+A364G+S388K+T480N;

V45L+A117S+L149R+G187S+S214Q+V258P+A276S+A364G+S388K;

A25W+V45L+A117S+L149R+S214Q+V258P+A276S+A364G+S388K;

V45L+A117S+L149R+S214Q+V258P+A276S+A364G+S388K;

V45L+A117S+L149R+S214Q+V258P+A276S+A364G+S388K+N476N;

A25W+V45L+L149R+S214E+V258P+A276Q+A364G;

V45L+A117S+L149R+S214Q+V258P+A276S+A364G+S388K+N476D;

V45L+L149R+S214Q+V258P+A276S+L281V+A364G;

V45L+A93S+L149R+A252S+H334D+A340G;

V45L+A93S+L149R+A252S+H334D;

V45L+A93S+L149R+A252S+H334D;

V45L+L149R+A252S+H334D;

V45L+N127D+L149R+V258P+A276S+A364G+S388K;

V45L+L149R+V258P+A276S+H358T+A364G+S388K;

V45L+L149R+V258P+A276S+A364G+S388K;

D9E+V45L+A117S+L149R+S214Q+V258P+A276S+A364G+S388K;

V45L+A117S+L149R+S214Q+V258P+A276S+A364G+S388K+S481S;

V45L+L149R+N168G+V258P+A276S+A364G;

V45L+L149R+T180Q+V258P+A276S+H358T+A364G+S388K;

V45L+L149R+G187L+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

A25W+V45L+L149R+V258P+A276S+A364G+S388K;

V45L+L149R+S214Q+V258P+A276S+H358T+A364G;

V45L+L149R+S173Y+V258P+A276S+A364G;

V45L+A117S+L149R+S214Q+H219V+V258P+A276S+A364G+S388K;

V45L+A93S+L149R+A252S+H334D;

V45L+A93S+L149R+A252S+H334D+A340G;

V45L+L149R+S190E+D446T+R459V;

A25W+V45L+L149R+V258P+A276S+H358T;

A25W+V45L+S92D+L149R+S214E+V258P+A276S+A364G;

A25W+V45L+L149R+S214E+V258P+A276S+V350L+A364G;

A25W+V45L+L149R+S214Q+V258P+A276S+A364G;

V45L+L149R+N168G+V258P+A276S+A364G+S388K;

V45L+A117S+L149R+V258P+A276S+A364G+S388K;

A25W+V45L+L149R+V258P+A276S+H358T+A364G;

V45L+L149R+V258P+N260H+A276S+A364G+S388K;

V45L+L149R+T215I+V258P+A276S+A364G+S388K;

A25W+V45L+L149R+V258P+A276Q+A364G;

V45L+T70R+A117S+L149R+C161L+S214Q+V258P+A276S+L281V+H358D+A364G+S388K+G394A+D470T+R483V;

A25W+V45L+L149R+S214E+V258P+A276S;

V45L+A117S+L149R+S214Q+V258P+A276S+A286S+H358D+A364G+S388K;

V45L+L149R+T211H+S214Q+V258P+A276S+A364G+S388K;

V45L+Q61K+L149R+V258P+A276S+H358T+A364G;

V45L+L149R+V258P+A276S+Q314A+H358T+A364G;

V45L+L149R+T180Q+V258P+A276S+A364G+S388K;

V45L+L149R+V258P+A276S+Q314A+A364G+S388K;

A25W+V45L+L149R+V258P+A276S+A286S+A364G;

V45L+L149R+S190E+D446T;

V45L+L149R+V258P+A276S+A364G;

V45L+L149R+A170S+V258P+A276S+A364G;

V45L+L149R+V258P+A276Q+A364G;

V45L+T70R+A117S+L149R+S214Q+V258P+A276S+L281V+H358D+A364G+S388K+G394A+D470T+R483V+C522A;

V45L+L149R+H219I+V258P+A276S+H358T+A364G;

V45L+L149R+V258P+A276S+V350L+A364G+S388K;

D11E+V45L+L149R+V258P+A276S+A364G+S388K;

V45L+D113T+L149R+V258P+A276S+A364G+S388K;

V45L+L149R+G187K+V258P+A276S+A364G+S388K;

M28P+V45L+L149R+V258P+A276S+A364G+S388K;

V45L+Q61R+A117S+L149R+S214Q+V258P+A276S+H358D+A364G+S388K+D470T+R483V;

A25W+V45L+L149R+S214Q+V258P+A276S;

V45L+A117S+L149R+S214Q+T215I+V258P+A276S+A364G+S388K;

A25W+V45L+L149R+V258P+A276S+S388K;

A25W+V45L+L149R+S214E+H219I+V258P+A276S+A364G;

V45L+A93S+L149R+V234P+A252S+H334D;

V45L+L149R+T211H+V258P+A276S+H358T+A364G+S388K;

A25W+V45L+A117S+L149R+S214E+V258P+A276S+A364G;

V45L+L149R+S173Y+V258P+A276S+A364G+S388K;

A25W+V45L+L149R+V258P+A276S+Q314A+A364G;

V45L+L149R+G187L+V258P+A276S+H358T+A364G;

V45L+L149R+T215V+V258P+A276S+H358T+A364G;

V45L+T70R+L149R+V258P+A276S+A364G+S388K;

V45L+N127D+L149R+V258P+A276S+A364G;

V45L+A117S+L149R+S214Q+V258P+A276S+A364G+S388K+Q496R;

V45L+L149R+W250Y+V258P+A276S+A364G;

V45L+L149R+V258P+A276S+V350L+H358T+A364G;

D9E+V45L+L149R+V258P+A276S+H358T+A364G;

V45L+L149R+T211H+V258P+A276S+H358T+A364G;

A25W+V45L+L149R+W250Y+A276Q+A364G;

V45L+L149R+V258P+A276S+L281V+A364G+S388K;

V45L+L149R+V258P+A276S+S282N+H358T+A364G;

V45L+L149R+T215I+V258P+A276S+H358T+A364G;

A25W+V45L+L149R+S173Y+V258P+A276S+A364G;

V45L+Q61K+L149R+V258P+A276S+A364G+S388K;

V45L+L149R+T180Q+V258P+A276S+A364G;

V45L+A117S+L149R+A170S+S214Q+V258P+A276S+A364G+S388K;

V45L+Q61R+A117S+L149R+S214Q+V258P+A276S+A364G+S388K;

A25W+V45L+L149R+S214E+T215V+V258P+A276S+A364G;

V45L+A117S+L149R+S214Q+V258P+A276S+A364G+S388K+V497P;

V45L+L149R+V258P+A276S+Q314A+A364G;

V45L+A117S+L149R+V258P+A276S+A364G;

V45L+A117S+L149R+V258P+A276S+H358T+A364G+S388K;

V45L+L149R+N168G+V258P+A276S+H358T+A364G+S388K;

A25W+V45L+Q61K+L149R+V258P+A276S+A364G;

V45L+L149R+S214E+V258P+A276S+L281V+A364G;

V45N+L149R+V258P+A276S+A364G+S388K;

V45L+L149R+V258P+A276S+A286E+H358T+A364G;

D9E+V45L+L149R+V258P+A276S+A364G+S388K;

V45L+S92D+L149R+V258P+A276S+A364G+S388K;

V45L+A117S+L149R+T180Q+S214Q+V258P+A276S+A364G+S388K;

V45L+A117S+L149R+S214Q+V258P+A276S+A364G+S388H;

V45L+A117S+L149R+S214Q+T215V+V258P+A276S+A364G+S388K;

V45L+A117S+L149R+S214Q+V258P+A276S+V350L+A364G+S388K;

A25W+V45L+L149R+S214E+T215I+V258P+A276S+A364G;

V45L+N127D+L149R+V258P+A276S+H358T+A364G+S388K;

A25W+V45L+L149R+V258P+A276S+A286E+A364G;

V45L+L149R+N168G+V258P+A276S+L281V+A364G;

V45L+Q61R+L149R+V258P+A276S+H358T+A364G;

V45L+L149R+G187S+V258P+A276S+A364G+S388K;

V45L+L149R+V258P+A276S+L281V+Q314A+A364G;

V45L+L149R+V258P+A276S+L281V+H358T+A364G;

V45L+L149R+A170S+V258P+A276S+H358T+A364G;

V45L+L149R+A170S+V258P+A276S+A364G+S388K;

V45L+L149R+T215V+V258P+A276S+A364G+S388K;

V45L+L149R+V258P+A276Q+L281V+A364G;

V45L+T70R+A117S+L149R+S214Q+C243S+V258P+A276S+L281V+H358D+A364G+S388K+G394A+D470T+R483V;

V45L+T70R+A117S+L149R+S214Q+V258P+A276S+L281V+H358D+A364G+S388K+G394A+D470T+R483V+C522V;

A25W+V45L+L149R+V258P+A276S+Q314A;

V45L+L149R+V258P+A276Q+H358T+A364G+S388K;

A25W+V45N+L149R+S214E+V258P+A276S+A364G;

V45L+L149R+V258P+A276S+A286E+A364G;

V45L+L149R+V258P+A276S+A286S+H358T+A364G;

A25W+V45L+L149R+G187S+V258P+A276S+A364G;

V45L+L149R+T180Q+V258P+A276S+L281V+A364G;

A25W+V45L+L149R+A276Q+L281V+A364G;

V45L+S92D+L149R+V258P+A276S+L281V+A364G;

V45L+Q61R+L149R+V258P+A276S+L281V+A364G;

V45L+L149R+A170S+V258P+A276S+L281V+A364G;

D11E+A25W+V45L+L149R+V258P+A276S+A364G;

A25W+V45L+Q61R+L149R+V258P+A276S+A364G;

A25W+V45L+L149R+T215V+V258P+A276S+A364G;

D9E+V45L+L149R+V258P+A276S+L281V+A364G;

A25W+V45L+L149R+V258P+A276S+V350L+A364G;

V45L+Q61K+A117S+L149R+S214Q+V258P+A276S+A364G+S388K;

V45L+A117S+L149R+S214Q+V258P+A276S+A364G+S388K+Y391V;

V45L+A117S+L149R+N168G+S214Q+V258P+A276S+A364G+S388K;

V45L+D113T+A117S+L149R+S214Q+V258P+A276S+A364G+S388K;

A25W+V45L+L149R+V258P+A276S+A286E;

A25W+V45L+L149R+V258P+A276S+L281V;

A25W+V45L+D113T+L149R+V258P+A276S;

V45L+L149R+H219V+V258P+A276S+L281V+A364G;

V45L+L149R+T180Q+V258P+A276S+H358T+A364G;

V45L+L149R+T215V+V258P+A276S+L281V+A364G;

A25W+V45L+L149R+V258P+A276S+S282N+A364G;

A25W+V45L+L149R+T215I+V258P+A276S+A364G;

A25W+V45L+L149R+H219I+V258P+A276S+A364G;

V45L+A117S+L149R+V258P+A276S+L281V+A364G;

D9E+A25W+V45L+L149R+V258P+A276S+A364G;

V45L+L149R+S214Y+V258P+A276S+A364G+S388K;

A25W+V45L+L149R+T180Q+V258P+A276S+A364G;

A25W+V45L+L149R+V258P+A276S+L281V+A364G;

V45L+N127D+L149R+V258P+A276S+L281V+A364G;

V45L+L149R+S173Y+V258P+A276S+H358T+A364G;

V45L+Q61R+L149R+V258P+A276S+A364G+S388K;

M28P+V45L+A117S+L149R+S214Q+V258P+A276S+A364G+S388K;

V45L+A117S+L149R+S214Q+V258P+A276S+A364G+S388A;

V45N+A117S+L149R+S214Q+V258P+A276S+A364G+S388K;

V45L+A117S+L149R+S214Q+V258P+A276S+A364G+S388K+T477K;

A25W+V45L+L149R+T180Q+V258P+A276S;

A25W+V45L+L149R+V258P+A276S+A286S;

A25W+V45L+L149R+G187S+S214E+V258P+A276S+A364G;

V45L+L149R+V258P+A276S+L281V+A286E+A364G;

A25W+V45L+L149R+W250Y+V258P+A276S+A364G;

A25W+V45L+L149R+V258P+N260H+A276S+A364G;

V45L+L149R+V258P+A276S+L281V+V350L+A364G;

A25W+V45L+L149R+A170S+V258P+A276S+A364G;

V45L+L149R+V258P+A276S+L281V+A286S+A364G;

V45L+L149R+V258P+A276S+L281V+S282N+A364G;

D11E+V45L+L149R+V258P+A276S+L281V+A364G;

V45L+Q61K+L149R+V258P+A276S+L281V+A364G;

V45L+L149R+T215I+V258P+A276S+L281V+A364G;

A25W+V45L+S92D+L149R+V258P+A276S+A364G;

V45L+L149R+S214Y+V258P+A276S+L281V+A364G;

V45L+L149R+T211H+V258P+A276S+L281V+A364G;

A25W+V45L+L149R+G187L+V258P+A276S+A364G;

V45L+S92D+L149R+V258P+A276S+H358T+A364G;

V45L+L149R+G187L+A276Q+L281V+A364G;

V45L+L149R+A276Q+L281V+V350L+A364G;

V45L+L149R+A276Q+L281V+A364G+S388K;

V45L+A117S+L149R+S214Y+V258P+A276S+A364G+S388K;

A25W+V45L+S92D+L149R+V258P+A276S;

A25W+V45L+L149R+G187L+V258P+A276S;

A25W+V45L+L149R+W250Y+V258P+A276S;

V45L+D113T+L149R+V258P+A276S+A364G;

V45L+L149R+G187K+V258P+A276S+H358T+A364G;

V45L+L149R+N163H+V258P+A276S+L281V+A364G;

A25W+V45L+T70R+L149R+V258P+A276S+A364G;

V45L+L149R+S173Y+V258P+A276S+L281V+A364G;

V45L+L149R+G187L+V258P+A276S+L281V+A364G;

V45L+L149R+G187K+V258P+A276S+L281V+A364G;

V45L+L149R+W250Y+V258P+A276S+H358T+A364G;

V45L+L149R+T211H+V258P+A276S+A364G+S388K;

V45L+L149R+N163H+V258P+A276S+A364G+S388K;

V45L+T70R+L149R+A276Q+L281V+A364G;

V45L+S92D+L149R+A276Q+L281V+A364G;

V45L+D113T+L149R+A276Q+L281V+A364G;

V45L+L149R+A276Q+L281V+Q314A+A364G;

V45L+A117S+L149R+S214Q+V258P+A276S+A364G+S388K+S389V;

A25W+V45L+Q61K+L149R+V258P+A276S;

D9E+A25W+V45L+L149R+V258P+A276S;

V45L+T70R+L149R+V258P+A276S+A364G;

V45L+L149R+V258P+A276S+A286S+A364G+S388K;

A25W+V45L+L149R+S214Y+V258P+A276S+A364G;

A25W+V45L+D113T+L149R+V258P+A276S+A364G;

V45L+T70R+L149R+V258P+A276S+L281V+A364G;

A25W+V45L+N127D+L149R+V258P+A276S+A364G;

V45L+T70R+L149R+V258P+A276S+H358T+A364G;

V45L+L149R+N163H+V258P+A276S+A364G;

V45L+L149R+T180Q+A276Q+L281V+A364G;

V45L+L149R+A276Q+L281V+A286S+A364G;

A25W+V45L+L149R+A170S+V258P+A276S;

A25W+V45L+T70R+L149R+V258P+A276S;

A25W+V45L+L149R+S214E+V258P+A276S+A286S+A364G;

V45L+L149R+V258P+A276S+A286S+H358T+A364G+S388K;

V45L+L149R+H219I+V258P+A276S+L281V+A364G;

A25W+V45L+A117S+L149R+V258P+A276S+A364G;

A25W+V45L+L149R+N168G+V258P+A276S+A364G;

V45L+L149R+N168G+A276Q+L281V+A364G;

V45L+L149R+S173Y+A276Q+L281V+A364G;

V45L+L149R+W250Y+A276Q+L281V+A364G;

V45L+A117S+L149R+S173Y+S214Q+V258P+A276S+A364G+S388K;

A25W+V45L+L149R+A276Q+A286E+A364G;

A25W+V45L+L149R+A276Q+A364G+S388K;

V45L+N127D+L149R+V258P+A276S+H358T+A364G;

A25W+V45L+L149R+V258P+A276S;

V45L+N127D+L149R+A276Q+L281V+A364G;

V45L+L149R+A276Q+L281V+A286E+A364G;

V45L+A117S+L149R+S214Q+V258P+A276S+A364G+S388K+V500T;

A25W+V45L+A117S+L149R+V258P+A276S;

A25W+V45L+L149R+H219V+V258P+A276S+A364G;

V45L+L149R+S173Y+V258P+A276S+H358T+A364G+S388K;

D11E+V45L+L149R+V258P+A276S+H358T+A364G;

V45L+A79C+L149R+V258P+A276S+N355G+A364G+S388K;

A24K+V45L+L149R+V258P+A276S+A364G;

V45L+L149R+V258P+A276S+S282N+A364G;

V45L+A117S+L149R+A276Q+L281V+A364G;

V45L+L149R+N260H+A276Q+L281V+A364G;

V45L+A117S+L149R+T211H+S214Q+V258P+A276S+A364G+S388K;

A25W+V45L+L149R+N168G+V258P+A276S;

A25W+V45L+L149R+G187S+V258P+A276S;

A25W+V45L+L149R+V258P+A276S+V350L;

A25W+V45L+Q61R+L149R+V258P+A276S;

A25W+V45L+L149R+G187L+S214E+V258P+A276S+A364G;

V45L+L149R+G187S+V258P+A276S+L281V+A364G;

A25W+V45L+L149R+N163H+V258P+A276S+A364G;

A25W+V45L+L149R+G187K+V258P+A276S+A364G;

V45N+L149R+V258P+A276S+L281V+A364G;

V45L+L149R+A276Q+L281V+A364G;

V45L+L149R+V258P+N260H+A276S+H358T+A364G;

V45L+L149R+A276Q+S282N+A364G;

V45L+S92D+A117S+L149R+S214Q+V258P+A276S+A364G+S388K;

A25W+V45L+L149R+V258P+A276S+S282N;

A25W+V45L+L149R+S173Y+V258P+A276S;

A25W+V45L+L149R+A276Q+A286S+A364G;

V45L+D113T+L149R+V258P+A276S+L281V+A364G;

V45L+L149R+S214Q+V258P+A276S+L281V+A364G+S388K;

V45L+D113T+L149R+V258P+A276S+H358T+A364G;

A25W+V45L+L149R+T215V+V258P+A276S;

V45L+L149R+N168G+V258P+A276S+H358T+A364G;

V45L+A117S+L149R+S214Q+V258P+A276S+A364G+S388K+A472L;

V45L+A117S+L149R+S214Q+V258P+A276S+A364G+S388K+S473K;

A25W+V45L+L149R+T215I+V258P+A276S;

A25W+V45L+L149R+T211H+V258P+A276S;

A25W+V45L+L149R+V258P+A276Q;

V45L+L149R+S214Q+T215V+V258P+A276S+A364G+S388K;

V45L+L149R+N163H+V258P+A276S+H358T+A364G+S388K;

V45L+A117S+L149R+S214Q+V258P+A276S+A364G+S388K+A499L;

V45L+L149R+V258P+A276S+V350L+A364G;

V45L+A79C+A117S+L149R+V258P+A276S+A364G+S388K;

A25W+V45L+D113T+L149R+A276Q+A364G;

A25W+V45N+L149R+V258P+A276S+A364G;

V45L+T70R+L149R+A276Q+A364G;

A25W+V45L+L149R+H219I+V258P+A276S;

V45L+A117S+L149R+S214Q+V258P+A276S+A364G+S388K+V498I;

M28P+V45L+L149R+V258P+A276S+A364G;

A25W+V45L+T70R+L149R+A276Q+A364G;

A25W+V45L+L149R+A276Q+Q314A+A364G;

V45L+L149R+A170S+V258P+A276S+H358T+A364G+S388K;

A25W+V45L+L149R+S214Y+V258P+A276S;

V45L+L149R+S214Y+V258P+A276S+H358T+A364G;

V45L+L149R+A276Q+L281V+H358T+A364G;

A25W+V45L+L149R+H219V+V258P+A276S;

V45L+A117S+L149R+N163H+S214Q+V258P+A276S+A364G+S388K;

D11E+A25W+V45L+L149R+V258P+A276S;

V45L+A117S+L149R+S214Q+V258P+A276S+A364G+S388K+N527H;

A25W+V45L+A117S+L149R+A276Q+A364G;

V45L+L149R+V258P+L281V+A364G;

A25W+V45L+L149R+G187L+A276Q+A364G;

V45N+L149R+V258P+A276S+H358T+A364G;

V45L+A117S+L149R+S214Q+V258P+A276S+N355G+H358D+A364G+S388K+D470T+R483V;

V45L+A117S+L149R+S214Q+V258P+A276S+H358D+A364G+S388H+D470T+R483V;

A25W+V45L+L149R+A276Q+A364G;

V45L+A117S+L149R+S214Q+V258P+A276S+H358D+A364G+S388K+D470T+R483V+V497E;

A25W+V45L+L149R+N163H+A276Q+A364G;

A25W+V45L+L149R+S173Y+A276Q+A364G;

A25W+V45L+L149R+A276Q+S282N+A364G;

A25W+V45L+L149R+A276Q+H358T+A364G;

A25W+V45L+L149R+T180Q+A276Q+A364G;

V45L+A117S+L149R+V258P+A276S+H358T+A364G;

V45L+L149R+A276Q+Q314A+A364G;

V45L+L149R+G187K+V258P+A364G;

V45L+L149R+A276Q+A286E+A364G;

A25W+V45L+N127D+L149R+A276Q+A364G;

A25W+V45L+L149R+A170S+A276Q+A364G;

A25W+V45L+N127D+L149R+V258P+A276S;

V45L+A117S+L149R+S214Q+V258P+A276S+A364G+S388K+T477V;

V45L+A117S+L149R+S214Q+V258P+A276S+A364G+S388K+G503L;

A25W+M28P+V45L+L149R+V258P+A276S+A364G;

V45L+L149R+A276Q+H358T+A364G;

A25W+V45L+L149R+N168G+A276Q+A364G;

A25W+V45L+L149R+N260H+A276Q+A364G;

V45L+A117S+L149R+G187L+S214Q+V258P+A276S+A364G+S388K;

V45L+A117S+L149R+N163H+S214Q+V258P+A276S+H358D+A364G+S388K+D470T+L530R;

V45L+A117S+L149R+S214Q+T215K+V258P+A276S+A364G+S388K;

A25W+V45L+L149R+G187K+V258P+A276S;

A25W+M28P+V45L+L149R+V258P+A276S;

A25W+V45N+L149R+V258P+A276S;

V45L+A117S+L149R+S214Q+V258P+A276S+N355G+H358D+A364G+S388K+D470T;

V45L+N127D+L149R+A276Q+A364G;

V45L+L149R+W250Y+A276Q+A364G;

V45L+L149R+W250Y+V258P+A364G;

V45L+L149R+N163H+A276Q+L281V+A364G;

V45L+A117S+L149R+S214Q+V258P+A276S+A364G+Q385C+S388K;

A25W+V45L+L149R+S214E+T215K+V258P+A276S+A364G;

M28P+V45L+L149R+V258P+A276S+L281V+A364G;

V45L+L149R+A276Q+A364G+S388K;

A25W+V45L+L149R+V258P+A364G;

M28P+V45L+L149R+V258P+A276S+H358T+A364G;

V45L+A117S+L149R+S214Q+V258P+A276S+N355G+H358D+A364G+S388K;

V45L+T70R+A117S+L149R+S214Q+V258P+A276S+L281V+H358D+A364G+S388K+G394A+D470T+R483V+C540V;

V45L+L149R+N168G+A276Q+A364G;

V45L+A117S+L149R+A276Q+A364G;

V45L+L149R+N163H+V258P+A276S+H358T+A364G;

V45L+L149R+T215K+V258P+A276S+H358T+A364G;

A24K+V45L+L149R+A276Q+A364G;

V45L+L149R+A276Q+A286S+A364G;

V45L+L149R+V258P+A364G;

V45L+L149R+H219I+V258P+A276S+A364G+S388K;

V45L+L149R+A170S+A276Q+A364G;

V45L+L149R+T180Q+A276Q+A364G;

V45L+L149R+A276Q+V350L+A364G;

V45L+L149R+V258P+S282N+A364G;

V45L+L149R+A276Q+A364G;

V45L+L149R+A276S+A364G;

V45L+L149R+T215K+V258P+A276S+A364G+S388K;

V45L+A117S+L149R+S214Q+V258P+A276S+N355G+A364G+S388K+D470T;

V45L+L149R+V258P+A276S;

A25W+V45L+L149R+A276S+A364G;

V45L+L149R+G187S+V258P+A364G;

V45L+S92D+L149R+V258P+A276S+H358T+A364G+S388K;

A25W+L149R+V258P+A276S+A364G;

V45L+L149R+G187L+A276Q+A364G;

A25W+V45L+V258P+A276S+A364G;

V45L+L149R+N260H+A276Q+A364G;

V45L+L149R+W250Y+V258R+A364G;

V45L+L149R+N163H+A276Q+A364G;

V45L+S92D+L149R+A276Q+A364G;

V45L+L149R+S173Y+A276Q+A364G;

V45L+S92D+L149R+V258P+A364G;

V45L+L149R+W250Y+V258P+A276S+L281V+A364G;

V45L+L149R+L281V+A364G;

V45L+D113T+L149R+A276Q+A364G;

L149R+V258P+A276S+A364G;

V45L+L149R+G187S+V258R+A364G;

V45L+V258P+A276S+A364G;

V45L+L149R+V258R+A364G+S388K;

A25W+V45L+L149R+A364G;

V45L+L149R+G187K+V258R+A364G;

V45L+L149R+V258P;

V45L+L149R+V258R+S282N+A364G;

V45L+L149R;

V45L+L149R+V258R+L281V+A364G;

V45L+L149R+A364G;

V45L+L149R+S214Q+V258P+A276S+N355G+A364G+S388K;

V45L+S92N+L149R+V258P+A364G;

V45L+A117S+L149R+S214Q+V258P+A276S+A286E+H358D+A364G+S388K;

A25W+V45L+L149R+S214E+V258P+A276S+N355G+A364G;

V45L+L149R+G187L+V258P+A276S+A364G;

V45L+L149R+V258R+A364G;

V45L+L149R+V258P+A364G+S388K;

A25W+V45L+L149R+A276Q+V350L+A364G;

V45L+L149R+H219V+V258P+A276S+A364G+S388K;

V45L+L149R+G187K+A364G;

V45L+L149R+G187S+A364G;

V45L+A117S+L149R+S214Q+V258P+A276S+N355G+A364G+S388K;

V45L+A117S+L149R+S214Q+V258P+A276S+H358D+A364G+Y386G+S388K+D470T+R483V;

V45L+L149R+W250Y;

V45L+S92N+L149R+V258R+A364G;

A25W+V45L+L149R+V258P+A276S+N355G+A364G;

V45L+L149R+V258P+A276S+N355G+A364G+S388K;

V45L+L149R+G187S;

V45N+L149R;

V45L+L149R+S282N;

L149R+A364G;

Y386G;

V258P+A276Q;

V258P+A276S;

V45L+A117S+L149R+S214Q+V258P+A276S+H358D+A364G+S388K+G394D+D470T+R483V;

A25W+L149R;

V45L+A276S;

V45L+S92D+L149R;

A25W+A276S;

Q385C;

V45L+L149R+W250Y+A364G;

V45L+A276Q;

V45L+V258P;

V45L+A117S+L149R+S214Q+V258P+A276S+H358D+A364G+Y386G+S388K;

A25W+V258P;

A25W+A276Q;

L149R+W250Y;

V45L+L149R+V258P+A276S+L281V+N355G+A364G;

V45L+S388K;

V45L+L149R+V258P+A276S+N355G+H358T+A364G;

V45L+A364G;

V45N+V258P;

A276S;

V258P+S388K;

A25W+V45L;

A276Q+S388K;

A276C;

A276Q;

A276F;

W250Y+A276Q;

A79C;

A276S+S388K;

A276Q+L281V;

V45L+L149R+V258P+A276S+N355G+A364G;

A276N;

W250Y+A276S;

A25W+S388K;

A276S+L281V;

G187S+A276Q;

A25W+V45L+L149R+V258P+A276S+N355G;

V45L;

S92D+A276S;

S19T;

L149R+V258R;

V45N+S388K;

A25W+V45L+S92D+L149R+A276Q+A364G;

V45L+A117S+L149R+S214Q+V258P+A276S+A364G+S388K+D470T+S501E H358D;

A25W+A364G;

V258P+L281V;

L149R;

V45L+L149R+H219V+V258P+A276S+H358T+A364G;

A25W;

V258R+A276S;

V45N;

G187S+A276S;

G187K+A276S;

I469T;

V45L+S92D;

V45L+L281V;

V45N+W250Y;

V258R+A276Q;

V45L+V258R;

W250Y+S388K;

A25W+V45L+L149R+A276Q+N355G+A364G;

V45N+G187K;

A25W+V45L+L149R+S214E+V258P+A276S+Q314A+A364G;

S388A;

A276T;

A25W+V258R;

G187K+S388K;

A25W+S92D;

L281V+S388K;

V45N+V258R;

S388H;

S92D;

D525L;

S214Q;

L281V+A364G;

A364G;

S388L;

S388R;

S473G;

S92D+A364G;

A25W+G187S;

A25W+G187K;

W250Y+V258P;

S92D+V258P;

S389V;

A20H;

W495E;

G187K+W250Y;

Y386Y;

V393H;

G187L;

Q61K;

V45N+S92D;

G187S+A364G;

G187S+L281V;

G187S;

A286E;

A276Y;

V258P;

Q496E;

G394A;

V393R;

G187S+S388K;

T366V;

S389S;

S92N;

N527H;

Q384A;

V497D;

D470T;

A18V;

Q496Q;

Q496R;

S214E;

P362P;

S389H;

V258R;

A276D;

L281V;

V451;

A117C;

N395N;

Y391Y;

A24H;

A472I;

D470K;

S473K;

S473S;

W495I;

V497E;

A24K;

V45T;

L471R;

A472L;

A20P;

N395D;

G394D;

G394Q;

G187S+V258R;

S92D+G187S;

V45C;

N476N;

G207G;

A364A;

D470A;

N474G;

D470V; and wherein the variant has xylanse activity and wherein the variant has at least 60%, at least 65%, at least 70%, at least 75%, at least 80%, at least 85%, at least 90%, at least 95%, at least 96%, at least 97%, at least 98%, or at least 99%, but less than 100% sequence identity to SEQ ID NO: 1, and wherein the variants have increased thermostability measured as increased melting temperature measured by TSA of at least 0.5° C. compared to the xylanase of SEQ ID NO: 1.

Paragraph 9. The xylanase variant according to any one of the preceding paragraphs, wherein the variant comprises at least one of the following substitutions or combinations of substitutions: V45L+A79C+A117S+L149R+S214Q+V258P+A276S+A364G+S388K+D470T+R483V;

V45L+A79C+A117S+L149R+S214Q+V258P+A276S+H358D+A364G+S388K+R483V;

V45L+Q61K+A117S+L149R+S214Q+V258P+A276S+H358D+A364G+S388K+D470T;

V45L+D95N+A117S+L149R+S214Q+V258P+A276S+H358D+A364G+S388K+G394A+D470T;

V45L+A117S+L149R+S214Q+V258P+A276S+A364G+S388K+G394A+D470T+R483V;

V45L+A93T+A117S+L149R+S214Q+V258P+A276S+H358D+A364G+S388K+D470T+A499Y;

V45L+A117S+L149R+S214Q+V258P+A276S+A364G+Q384A+S388K+D470T+R483V;

V45L+A79C+A117S+L149R+S214Q+V258P+A276S+H358D+A364G+S388K;

V45L+A117S+L149R+S214Q+V258P+A276S+A364G+S388K+D470T+R483V;

V45L+A117S+L149R+S214Q+V258P+A276S+H358D+A364G+S388K+D470T;

V45L+A79C+A117S+L149R+S214Q+V258P+A276S+A364G+S388K+D470T;

V45L+D95N+A117S+L149R+S214Q+V258P+A276S+H358D+A364G+S388K+S473G;

V45L+T70R+D95N+A117S+L149R+S214Q+V258P+A276S+L356M+H358D+A364G+S388K;

V45L+A117S+L149R+S214Q+V258P+A276S+H358D+A364G+S388K+D470A;

V45L+A117S+L149R+S214Q+V258P+A276S+H358D+A364G+S388K+L478N;

V45L+A117S+L149R+S214Q+V258P+A276S+H358D+A364G+S388K+N395D;

V45L+A117S+L149R+S214Q+V258P+A276S+H358D+A364G+S388K+T477K;

V45L+A117S+L149R+S214Q+V258P+A276S+L281V+H358D+A364G+S388K;

D11E+V45L+A117S+L149R+S214Q+V258P+A276S+H358D+A364G+S388K;

V45L+L149R+V258P+A276S+H358T+A364G;

A25W+V45L+L149R+V258P+A276S+A364G;

V45L+L149R+S214Q+V258P+A276S+A364G+S388K;

V45L+L149R+V258P+A276S+L281V+A364G;

V45L+N127D+L149R+V258P+A276S+A364G+S388K;

V45L+L149R+V258P+A276S+H358T+A364G+S388K;

V45L+L149R+V258P+A276S+A364G+S388K;

V45L+L149R+W250Y+V258P+A276S+A364G+S388K;

V45L+L149R+V258P+A276S+A286E+A364G+S388K;

V45L+L149R+V258P+A276S+S282N+A364G+S388K;

V45L+L149R+S214Q+V258P+A276S+H358T+A364G;

A25W+V45L+L149R+V258P+A276Q+A364G;

V45L+L149R+V258P+A276S+A364G;

A25W+V45L+L149R+W250Y+A276Q+A364G;

A25W+V45L+L149R+A276Q+L281V+A364G;

A25W+V45L+L149R+A276Q+A286E+A364G;

A25W+V45L+L149R+A276Q+A364G+S388K;

V45L+L149R+V258P+L281V+A364G;

A25W+V45L+L149R+A276Q+A364G;

V45L+L149R+V258P+A364G;

V45L+L149R+V258P+S282N+A364G;

V45L+L149R+A276Q+A364G;

V45L+L149R+A276S+A364G;

V45L+L149R;

V45L+L149R+A364G;

V45L+L149R+V258R+A364G;

V258P+A276Q;

V258P+A276S;

V45L+A276S;

V45L+A276Q;

L149R+W250Y;

V258P+S388K;

A276Q+S388K;

W250Y+A276Q;

A276S+S388K;

A276Q+L281V;

W250Y+A276S;

A276S+L281V;

G187S+A276Q;

V45L+L281V;

V45L;

L149R;

A79C;

V45N;

V45L+D11E;

L149R+S388K;

L149R+D11E;

A364G+D11E;

S388K+D11E; and wherein the variant has xylanse activity and wherein the variant has at least 60%, at least 65%, at least 70%, at least 75%, at least 80%, at least 85%, at least 90%, at least 95%, at least 96%, at least 97%, at least 98%, or at least 99%, but less than 100% sequence identity to SEQ ID NO: 1, and wherein the variants have increased thermostability measured as increased melting temperature measured by n-DSF of at least 0.5° C. compared to the xylanase of SEQ ID NO: 1.

Paragraph 10. The xylanase variant according to any one of the preceding paragraphs, wherein the parent polypeptide has at least 60%, e.g., at least 65%, at least 70%, at least 75%, at least 80%, at least 85%, at least 90%, at least 95%, at least 96%, at least 97%, at least 98%, at least 99% but less than 100% sequence identity to the polypeptide of SEQ ID NO: 1.

Paragraph 11. The xylanase variant according to any one of the preceding paragraphs, wherein said parent polypeptide comprises or consists of the polypeptide of SEQ ID NO: 1.

Paragraph 12. The xylanase variant according to any one of the preceding paragraphs, wherein the substitution is selected from the group consisting of A, C, D, E, F, G, H, I, K, L, M, N, P, Q, R, S, T, V, W and Y, with the proviso that the substituted amino acid residue is different from the naturally-occurring amino acid residue in that position.

Paragraph 13. The xylanase variant according to any one of the preceding paragraphs, wherein the number of substitutions is 1-20, e.g., 1-10 and 1-5, such as 1, 2, 3, 4, 5, 6, 7, 8, 9 or 10 substitutions.

Paragraph 14. A polynucleotide encoding the xylanase variant of any of paragraphs 1-13.

Paragraph 15. A nucleic acid construct comprising the polynucleotide of paragraph 14.

Paragraph 16. An expression vector comprising the polynucleotide of paragraph 14.

Paragraph 17. A host cell comprising the polynucleotide of paragraph 14.

Paragraph 18. The host cell according to paragraph 17, wherein the host cell is a yeast cell, particularly a Saccharomyces, such as Saccharomyces cerevisiae.

Paragraph 19. A method of producing a xylanase variant of any one of the preceding paragraphs, comprising cultivating the host cell of paragraphs 17 or 18 under conditions conducive for production of the polypeptide.

Paragraph 20. The method according to paragraph 19, further comprising recovering the polypeptide.

Paragraph 21. A method of obtaining a xylanase variant, comprising (a) introducing into a parent xylanase a substitution at one or more positions corresponding to positions 45, 149, 364, 258, 276, and 388 of SEQ ID NO: 1, wherein the xylanase variant has xylanase activity and has at least 60, e.g., at least 65%, at least 70%, at least 75%, at least 80%, at least 85%, at least 90%, at least 95%, at least 96%, at least 97%, at least 98%, or at least 99% sequence identity to SEQ ID NO: 1; and (b) optionally recovering the xylanase variant.

Paragraph 22. The method according to paragraph 21 further comprising (a) substitution at one or more positions corresponding to positions to 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1, wherein the xylanase variant has xylanase activity and has at least 60, e.g., at least 65%, at least 70%, at least 75%, at least 80%, at least 85%, at least 90%, at least 95%, at least 96%, at least 97%, at least 98%, or at least 99% sequence identity to SEQ ID NO: 1; and (b) optionally recovering the xylanase variant.

Paragraph 23. A whole broth formulation or cell culture composition comprising a xylanase variant of any of paragraphs 1-13.

Paragraph 24. A composition comprising the xylanase variant of paragraphs 1-13.

Paragraph 25. The composition according to paragraph 24, further comprising one or more enzymes selected from the group consisting of: cellulase, hemicellulase, cellulases (EC 3.2.1.4), xylanases (EC 3.2.1.8) arabinofuranosidases (EC 3.2.1.55 (Non-reducing end alpha-L-arabinofuranosidases); EC 3.2.1.185 (Non-reducing end beta-L-arabinofuranosidases) cellobiohydrolase I (EC 3.2.1.150), cellobiohydrolase II (E.C. 3.2.1.91), cellobiosidase (E.C. 3.2.1.176), beta-glucosidase (E.C. 3.2.1.21), beta-xylosidases (EC 3.2.1.37) and combination thereof.

Paragraph 26. A method for increasing the starch yield and/or gluten yield from corn kernels in a wet milling process, the method comprising contacting corn kernels or a fraction of the corn kernels, particularly a fiber rich fraction, with an effective amount of a xylanase variant of paragraph 1-13 and optionally one or more hydrolytic enzymes.

Paragraph 27. The method according to paragraph 26, comprising the steps of:

a) soaking the corn kernels in water to produce soaked kernels;

b) grinding the soaked kernels to produce ground kernels;

c) separating germ from the ground kernels to produce a corn kernel mass comprising fiber, starch and gluten; and

d) subjecting the corn kernel mass to a fiber washing procedure separating starch and gluten from the fiber;

wherein at least a xylanase variant of paragraph 1-13 is present/added before or during step d).

Paragraph 28. The method of paragraph 27, further comprising the steps of:

e) separating the starch from the gluten; and optionally

f) washing the starch.

Paragraph 29. The method according to any of paragraphs 26-28, wherein the one or more hydrolytic enzymes comprises one or more cellulases.

Paragraph 30. The method according to paragraph 29, wherein the cellulase(s) comprises one or more enzyme selected from the group consisting of an endoglucanase (EG), and a cellobiohydrolase (CBH).

Paragraph 31. The method of paragraph 30, wherein the cellulase(s) comprises one or more enzyme selected from the group consisting of an endoglucanase, a cellobiohydrolase I, a cellobiohydrolase II, or a combination thereof.

Paragraph 32. The method according to any of the paragraphs 27-31, wherein the corn kernel mass comprising fiber, starch and gluten from step c) is subjected to a further grinding step, preferably a fine grinding step before the fiber washing procedure in step d).

Paragraph 33. The method according to any of the preceding paragraphs 26-32, wherein the effective amount of one or more hydrolytic enzymes admixed with one or more fractions of said corn kernel mass, is between 0.005-0.5 kg enzyme protein/metric tonne corn kernels entering the wet milling process.

Paragraph 34. Use of xylanase variant of paragraphs 1-13 in a method to increase the starch yield and/or gluten yield from corn kernels in a wet milling process.

Paragraph 35. A process for producing fermentation products from starch-containing material comprising the steps of:

i) saccharifying the starch-containing material using a carbohydrate-source generating enzyme at a temperature below the initial gelatinization temperature;

ii) fermenting using a fermenting organism;

wherein at least one or more xylanase variant(s) of paragraphs 1-13 are present or added during fermentation or simultaneous saccharification and fermentation.

Paragraph 36. A process for producing a fermentation product from starch-containing material comprising the steps of: (a) liquefying starch-containing material above the initial gelatinization temperature of said starch-containing material in the presence of an alpha amylase; (b) saccharifying the liquefied material; and (c) fermenting with a fermenting organism; wherein step (b) is carried out in the presence of at least a glucoamylase and the variant alpha-amylase of any of paragraphs 1-13.

Paragraph 37. The process of any of paragraphs 35-36, wherein saccharification and fermentation is carried out simultaneously.

Paragraph 38. The process of any of the paragraphs 35-37, wherein the host cell of paragraph 18 is applied as the fermenting organism.

Paragraph 39. The process of paragraph 38, wherein the host cell further is expressing a glucoamylase.

Paragraph 40. The process of any of paragraphs 35-39, wherein the starch-containing material is corn and the fermentation product is ethanol.

### EXAMPLES

### Example 1: Construction of Variants by Site-Directed Mutagenesis

Site-directed variants were constructed of the Chryseobacterium sp., 10696 xylanase (SEQ ID NO: 1), comprising specific substitutions according to the invention. SEQ ID NO: 2 encodes the Chryseobacterium sp., 10696 xylanase of SEQ ID NO: 1. The variants were made by traditional cloning of DNA fragments (Sambrook et al., Molecular Cloning: A Laboratory Manual, 2nd Ed., Cold Spring Harbor, 1989) using PCR together with properly designed mutagenic oligonucleotides that introduced the desired mutations in the resulting sequence. Mutagenic oligos were designed corresponding to the DNA sequence flanking the desired site(s) of mutation, separated by the DNA base pairs defining the insertions/deletions/substitutions, and purchased from an oligo vendor such as Macrogen, Inc.

### Example 2: Protein Thermal Unfolding Analysis (TSA, Thermal Shift Assay) of Culture Supernatant Samples

Culture supernatants grown in 96-well plate (MicroAmpR fast 96-well plate, Applied Biosystems) was clarified by centrifugation at 3000×g for 30 min at 4° C. In a 96-well PCR plate, pre-clarified supernatant (5 μl) was mixed with reporter dye solution (2.5 μl) [1 μl of Sypro orange (S5692, Sigma) in 125 μl of MilliQ water]. Final reaction volume was made upto 30 μl by adding 22.5 μl of dilution buffer [sodium acetate buffer (50 mM) was made by adding 4.1 g of sodium acetate (Sigma, S2889) in 1 L of Milli-Q water and adjusting the pH to 4.0 using acetic acid]. A plate was sealed with a plate sealer (MicroAmpR optical adhesive film, Applied Biosystems) and kept in a TSA instrument. In each run, purified xylanase-WT and culture supernatant-WT were used as controls. The results in table 1 show that the xylanase variants have improved thermostability relative to the wild-type xylanase (SEQ ID NO: 1).

TABLE 1
TSA data of xylanase variants of SEQ ID NO: 1

| | Tm |
|---|---|
| Variant(s) | (° C.) |
| WT | 44.3 |
| V45L + Q61K + T70R + A117S + L149R + S214Q + V258P + | 61.4 |
| A276S + L281V + A286E + H358D + A364G + Q384A + | |
| S388K + V393H + D470T + R483V | |
| V45L + Q61K + T70R + A117S + L149R + S214Q + V258P + | 61.4 |
| A276S + L281V + H358D + A364G + Q384A + S388K + | |
| G394Q + D470T + R483V | |
| V45L + Q61K + T70R + A117S + L149R + S214Q + V258P + | 61.3 |
| A276S + L281V + H358D + A364G + Q384A + S388K + | |
| G394A + D470T + R483V | |
| V45L + Q61K + T70R + A117S + L149R + S214Q + V258P + | 61.3 |
| A276S + L281V + H358D + A364G + Q384A + S388K + | |
| V393H + D470T + R483V | |
| V45L + Q61K + T70R + A117S + L149R + S214Q + V258P + | 61.2 |
| A276S + L281V + H358D + A364G + Q384A + S388K + | |
| V393H + D470T + R483V | |
| V45L + Q61K + T70R + A117S + L149R + S214Q + V258P + | 61.1 |
| A276S + L281V + H358D + A364G + Q384A + S388K + | |
| G394A + D470T + R483V | |
| V45L + Q61K + T70R + A117S + L149R + S214Q + V258P + | 61.1 |
| A276S + L281V + H358D + A364G + Q384A + S388K + | |
| V393H + D470T + R483V | |
| V45L + Q61K + T70R + A117S + L149R + S214Q + V258P + | 61.1 |
| A276S + L281V + H358D + A364G + Q384A + S388K + | |
| G394A + D470T + R483V | |
| V45L + Q61K + T70R + A117S + L149R + S214Q + V258P + | 61.1 |
| A276S + L281V + A286E + H358D + A364G + Q384A + | |
| S388K + V393H + D470T + R483V | |
| V45L + Q61K + T70R + A117S + L149R + S214Q + V258P + | 61.0 |
| A276S + L281V + H358D + A364G + Q384A + S388K + | |
| V393H + D470T + R483V | |
| V45L + Q61K + T70R + A117S + L149R + S214Q + V258P + | 61.0 |
| A276S + L281V + H358D + A364G + Q384A + S388K + | |
| G394A + D470T + R483V | |
| V45L + A79C + A117S + L149R + S214Q + V258P + A276S + | 60.6 |
| H358D + A364G + S388K + D470T + L530R | |
| V45L + A79C + A117S + L149R + S214Q + V258P + A276S + | 60.6 |
| H358D + A364G + S388K + D470T + R483V | |
| V45L + T70R + A117S + L149R + S214Q + V258P + A276S + | 60.5 |
| L281V + H358D + A364G + S388K + G394A + D470T + | |
| R483V | |
| V45L + T70R + A117S + L149R + S214Q + V258P + A276S + | 60.5 |
| L281V + H358D + A364G + S388K + G394A + D470T + | |
| R483V | |
| V45L + T70R + A117S + L149R + S214Q + V258P + A276S + | 60.4 |
| L281V + H358D + A364G + S388K + G394A + D470T + | |
| R483V | |
| V45L + T70R + A117S + L149R + S214Q + V258P + A276S + | 60.4 |
| L281V + H358D + A364G + S388K + G394A + D470T + | |
| R483V | |
| V45L + T70R + A117S + L149R + S214Q + V258P + A276S + | 60.3 |
| L281V + H358D + A364G + S388K + G394A + D470T + | |
| R483V | |
| V45L + T70R + A117S + L149R + S214Q + V258P + A276S + | 60.3 |
| L281V + H358D + A364G + S388K + G394A + D470T + | |
| R483V | |
| V45L + Q61K + A117S + L149R + S214Q + V258P + A276S + | 60.2 |
| L281V + H358D + A364G + S388K + G394A + D470T + | |
| R483V + L530R | |
| V45L + T70R + A117S + L149R + S214Q + V258P + A276S + | 60.2 |
| L281V + H358D + A364G + S388K + G394A + D470T + | |
| R483V | |
| V45L + T70R + A117S + L149R + S214Q + V258P + A276S + | 60.2 |
| L281V + H358D + A364G + S388K + G394A + D470T + R483V | |
| V45L + T70R + A117S + L149R + S214Q + V258P + A276S + | 60.1 |
| L281V + H358D + A364G + S388K + G394A + D470T + R483V | |
| V45L + T70R + A117S + L149R + S214Q + V258P + A276S + | 60.1 |
| L281V + H358D + A364G + S388K + G394A + D470T + R483V | |
| V45L + Q61K + A117S + L149R + S214Q + V258P + A276S + | 59.9 |
| L281V + H358D + A364G + S388K + G394A + D470T + L530R | |
| V45L + Q61K + A117S + L149R + S214Q + V258P + A276S + | 59.7 |
| L281V + H358D + A364G + S388K + G394A + D470T + | |
| R483V + L530R | |
| V45L + Q61K + A117S + L149R + S214Q + V258P + A276S + | 59.6 |
| L281V + H358D + A364G + S388K + G394A + D470T + | |
| R483V + L530R | |
| V45L + Q61K + A117S + L149R + S214Q + V258P + A276S + | 59.6 |
| L281V + H358D + A364G + S388K + D470T + | |
| R483V + L530R | |
| V45L + Q61K + A117S + L149R + S214Q + V258P + A276S + | 59.6 |
| L281V + H358D + A364G + S388K + G394A + D470T + | |
| R483V + L530R | |
| V45L + Q61K + A117S + L149R + S214Q + V258P + A276S + | 59.6 |
| L281V + H358D + A364G + S388K + D470T + | |
| R483V + L530R | |
| V45L + A117S + L149R + G187K + S214Q + V258P + A276S + | 59.5 |
| A286E + H358D + A364G + Q384A + S388K + V393H + | |
| D470T + R483V | |
| V45L + A117S + L149R + G187K + S214Q + V258P + A276S + | 59.4 |
| A286E + H358D + A364G + Q384A + S388K + N395D + | |
| D470T + R483V | |
| V45L + A117S + L149R + G187K + S214E + T215I + V258P + | 59.2 |
| A276S + A286E + H358D + A364G + S388K + V393H + | |
| D470T + R483V | |
| V45L + A117S + L149R + G187K + S214E + T215I + V258P + | 59.1 |
| A276S + A286E + H358D + A364G + S388K + N395D + | |
| D470T + R483V | |
| V45L + A117S + L149R + S214Q + V258P + A276S + H358D + | 59.1 |
| A364G + Q385C + S388K + D470T + R483V | |
| V45L + Q61K + A117S + L149R + S214Q + V258P + A276S + | 58.9 |
| H358D + A364G + S388K + D470T + R483V | |
| V45L + A79C + A117S + L149R + S214Q + V258P + A276S + | 58.9 |
| A364G + S388K + D470T + R483V | |
| V45L + A117S + L149R + S214Q + V258P + A276S + L281V + | 58.8 |
| H358D + A364G + S388K + D470T + R483V | |
| V45L + A117S + L149R + S214Q + V258P + A276S + H358D + | 58.8 |
| A364G + T366I + S388K + D470T + R483V | |
| V45L + T70R + A117S + L149R + S214Q + V258P + A276S + | 58.8 |
| L281V + H358D + A364G + S388K + G394A + D470T + R483V | |
| V45L + A117S + L149R + S214Q + V258P + A276S + H358D + | 58.8 |
| A364G + Q384A + S388K + D470T + L530R | |
| V45L + Q61K + A117S + L149R + S214Q + V258P + A276S + | 58.8 |
| H358D + A364G + S388K + D470T + L530R | |
| V45L + T70R + A117S + L149R + S214Q + V258P + A276S + | 58.8 |
| H358D + A364G + S388K + D470T + R483V | |
| V45L + A117S + L149R + S214Q + V258P + A276S + H358D + | 58.8 |
| A364G + S388K + V393H + D470T + R483V | |
| V45L + A117S + L149R + S214Q + V258P + A276S + H358D + | 58.8 |
| A364G + S388K + G394A + D470T + R483V | |
| V45L + A117S + L149R + S214Q + V258P + A276S + H358D + | 58.7 |
| A364G + S388K + V393H + D470T + L530R | |
| V45L + A117S + L149R + S214Q + V258P + A276S + A286E + | 58.7 |
| H358D + A364G + S388K + D470T + R483V | |
| D11E + V45L + A117S + L149R + S214Q + V258P + A276S + | 58.7 |
| H358D + A364G + S388K + D470T + R483V | |
| V45L + T70R + A117S + L149R + S214Q + V258P + A276S + | 58.7 |
| L281V + H358D + A364G + S388K + G394A + C468V + | |
| D470T + R483V | |
| V45L + A79C + A117S + L149R + S214Q + V258P + A276S + | 58.7 |
| H358D + A364G + S388K + D470T | |
| V45L + A79C + L149R + S214E + V258P + A276S + A364G + | 58.6 |
| S388K | |
| V45L + A117S + L149R + G187L + S214Q + V258P + A276S + | 58.6 |
| H358D + A364G + S388K + D470T + L530R | |
| V45L + A117S + L149R + S214Q + V258P + A276S + H358D + | 58.6 |
| A364G + Q384A + S388K + D470T + R483V | |
| V45L + A117S + L149R + G187K + S214Q + V258P + A276S + | 58.6 |
| H358D + A364G + S388K + D470T + R483V | |
| V45L + A117S + L149R + S214Q + V258P + A276S + L281V + | 58.6 |
| H358D + A364G + S388K + D470T + L530R | |
| V45L + A117S + L149R + S214E + V258P + A276S + H358D + | 58.6 |
| A364G + S388K + D470T + R483V | |
| V45L + A117S + L149R + G187L + S214Q + V258P + A276S + | 58.6 |
| H358D + A364G + S388K + D470T + R483V | |
| V45L + A117S + L149R + S214Q + V258P + A276S + H358D + | 58.6 |
| A364G + S388K + G394Q + D470T + R483V | |
| V45L + A117S + L149R + S214Q + V258P + A276S + H358D + | 58.5 |
| A364G + S388K + G394A + D470T + L530R | |
| V45L + A117S + L149R + S214Q + V258P + A276S + H358D + | 58.5 |
| A364G + T366V + S388K + D470T + L530R | |
| V45L + A117S + L149R + S214Q + V258P + A276S + H358D + | 58.5 |
| A364G + S388K + G394Q + D470T + L530R | |
| V45L + A117S + L149R + S214Q + V258P + A276S + S282N + | 58.5 |
| H358D + A364G + S388K + D470T + R483V | |
| V45L + A117S + L149R + S214Q + V258P + A276S + H358D + | 58.5 |
| A364G + S388K + N395D + D470T + R483V | |
| V45L + A117S + L149R + S214Q + V258P + A276S + H358D + | 58.5 |
| A364G + S388K + D470T + N476D + R483V | |
| V45L + A117S + L149R + S214Q + V258P + A276S + V350L + | 58.5 |
| H358D + A364G + S388K + D470T + R483V | |
| V45L + A117S + L149R + S214Q + V258P + A276S + H358D + | 58.5 |
| A364G + S388K + D470T + T477K + R483V | |
| V45L + A117S + L149R + S214Q + V258P + A276S + H358D + | 58.5 |
| A364G + S388K + D470T + R483V + Q496R | |
| V45L + T70R + A117S + L149R + S214Q + V258P + A276S + | 58.5 |
| L281V + H358D + A364G + S388K + G394A + C468A + | |
| D470T + R483V | |
| V45L + A117S + L149R + S214Q + V258P + A276S + H358D + | 58.5 |
| A364G + S388K + D470T + R483V + W495E | |
| V45L + A117S + L149R + S214Q + V258P + A276S + A286E + | 58.4 |
| H358D + A364G + S388K + D470T + L530R | |
| V45L + A117S + L149R + G187K + S214Q + V258P + A276S + | 58.4 |
| H358D + A364G + S388K + D470T + L530R | |
| V45L + A117S + L149R + S214Q + V258P + A276S + H358D + | 58.4 |
| A364G + S388K + Y391V + D470T + R483V | |
| V45L + A117S + L149R + S214Q + V258P + A276S + H358D + | 58.4 |
| A364G + S388K + D470T + R483V | |
| D9E + V45L + A117S + L149R + S214Q + V258P + A276S + | 58.4 |
| H358D + A364G + S388K + D470T + L530R | |
| V45L + A117S + L149R + S214Q + V258P + A276S + H358D + | 58.4 |
| A364G + S388K + D470T + T477V + R483V | |
| V45L + A117S + L149R + S214Q + V258P + A276S + H358D + | 58.4 |
| A364G + S388K + D470T + N476N + R483V | |
| V45L + A117S + L149R + S214Q + V258P + A276S + H358D + | 58.4 |
| A364G + S388K + V393R + D470T + R483V | |
| V45L + A117S + L149R + S214Q + V258P + A276S + H358D + | 58.3 |
| A364G + S388K + D470T + T480L + L530R | |
| V45L + A117S + L149R + S214Q + V258P + A276S + H358D + | 58.3 |
| A364G + S388K + D470T + S481A + L530R | |
| V45L + A117S + L149R + S214Q + W250Y + V258P + A276S + | 58.3 |
| H358D + A364G + S388K + D470T + R483V | |
| V45L + A117S + L149R + S214Q + V258P + A276S + H358D + | 58.3 |
| A364G + S388K + D470T + S481S + R483V | |
| V45L + A117S + L149R + S173Y + S214Q + V258P + A276S + | 58.3 |
| H358D + A364G + S388K + D470T + R483V | |
| V45L + T70R + A117S + L149R + S214Q + C243V + V258P + | 58.3 |
| A276S + L281V + H358D + A364G + S388K + G394A + | |
| D470T + R483V | |
| V45L + A117S + L149R + T180Q + S214Q + V258P + A276S + | 58.2 |
| H358D + A364G + S388K + D470T + L530R | |
| D11E + V45L + A117S + L149R + S214Q + V258P + A276S + | 58.2 |
| H358D + A364G + S388K + D470T + L530R | |
| V45L + A117S + L149R + S214E + V258P + A276S + H358D + | 58.2 |
| A364G + S388K + D470T + L530R | |
| V45L + A117S + L149R + S214Q + V258P + A276S + H358D + | 58.2 |
| A364G + S388K + D470T + N476D + L530R | |
| V45L + A117S + L149R + S214Q + V258P + A276S + A286S + | 58.2 |
| H358D + A364G + S388K + D470T + R483V | |
| V45L + A117S + L149R + S214Q + V258P + A276S + H358D + | 58.2 |
| A364G + S388K + D470T + R483V + Q496Q | |
| V45L + A117S + L149R + S214Q + V258P + A276Q + H358D + | 58.2 |
| A364G + S388K + D470T + R483V | |
| V45L + A117S + L149R + S214Q + V258P + A276S + H358D + | 58.2 |
| A364G + S388K + D470T + R483V + Q496E | |
| V45L + Q61R + A117S + L149R + S214Q + V258P + A276S + | 58.2 |
| H358D + A364G + S388K + D470T + L530R | |
| V45L + A117S + L149R + S214Q + V258P + A276S + Q314A + | 58.2 |
| H358D + A364G + S388K + D470T + L530R | |
| V45L + A117S + L149R + S214Q + V258P + A276Q + H358D + | 58.2 |
| A364G + S388K + D470T + L530R | |
| V45L + A117S + L149R + S214Q + V258P + A276S + H358D + | 58.2 |
| A364G + S388K + D470T + L478P + R483V | |
| A25W + V45L + A117S + L149R + S214Q + V258P + A276S + | 58.2 |
| H358D + A364G + S388K + D470T + R483V | |
| V45L + A117S + L149R + T180Q + S214Q + V258P + A276S + | 58.2 |
| H358D + A364G + S388K + D470T + R483V | |
| V45L + A117S + L149R + S214Q + V258P + A276S + H358D + | 58.2 |
| A364G + S388K + D470T + R483V + L484K | |
| V45L + A117S + L149R + S214Q + V258P + A276S + H358D + | 58.1 |
| A364G + S388K + D470T + N474G + L530R | |
| V45L + A117S + L149R + S214Q + V258P + A276S + H358D + | 58.1 |
| A364G + S388K + N395D + D470T + L530R | |
| V45L + A117S + L149R + S214Q + V258P + A276S + H358D + | 58.1 |
| A364G + T366V + S388K + D470T + R483V | |
| V45L + A117S + L149R + N168G + S214Q + V258P + A276S + | 58.1 |
| H358D + A364G + S388K + D470T + R483V | |
| V45L + A117S + L149R + S214Q + V258P + A276S + H358D + | 58.1 |
| A364G + S388K + D470T + R483V + L484S | |
| V45L + A117S + L149R + S214Q + V258P + A276S + H358D + | 58.1 |
| A364G + S388K + D470T + L530R | |
| V45L + A117S + L149R + S214Q + V258P + A276S + H358D + | 58.1 |
| A364G + S388K + D470T + R483V + L484D | |
| V45L + A117S + L149R + S214Q + V258P + A276S + H358D + | 58.1 |
| A364G + S388K + D470T + R483V + S528H | |
| V45L + A117S + L149R + S214Q + V258P + A276S + H358D + | 58.1 |
| A364G + S388K + D470T + R483V + S501N | |
| V45L + A117S + L149R + S173Y + S214Q + V258P + A276S + | 58.1 |
| H358D + A364G + S388K + D470T + L530R | |
| V45L + A117S + L149R + S214Q + V258P + A276S + H358D + | 58.0 |
| A364G + S388K + D470T + R483V + A499L | |
| V45L + S92D + A117S + L149R + S214Q + V258P + A276S + | 58.0 |
| H358D + A364G + S388K + D470T + R483V | |
| V45L + A117S + L149R + S214Q + V258P + A276S + Q314A + | 58.0 |
| H358D + A364G + S388K + D470T + R483V | |
| V45L + A79C + L149R + S214Q + V258P + A276S + A364G + | 58.0 |
| S388K | |
| V45L + A117S + L149R + S214Q + V258P + A276S + N355G + | 58.0 |
| H358D + A364G + S388K + D470T + L530R | |
| V45L + A117S + L149R + S214Q + V258P + A276S + V350L + | 58.0 |
| H358D + A364G + S388K + D470T + L530R | |
| V45L + A117S + L149R + S214Q + V258P + A276S + H358D + | 58.0 |
| A364G + S388K + D470T + S481S + L530R | |
| V45L + A117S + L149R + S214Q + V258P + A276S + H358D + | 58.0 |
| A364G + S388K + D470T + Q496E + L530R | |
| V45L + A117S + L149R + N168G + S214Q + V258P + A276S + | 58.0 |
| H358D + A364G + S388K + D470T + L530R | |
| V45L + A117S + L149R + S214Q + V258P + A276S + H358D + | 58.0 |
| A364G + S388K + D470T + V498I + L530R | |
| V45L + A117S + L149R + S214Q + V258P + A276S + H358D + | 58.0 |
| A364G + S388K + D470A + R483V | |
| V45N + A117S + L149R + S214Q + V258P + A276S + H358D + | 58.0 |
| A364G + S388K + D470T + R483V | |
| V45L + A117S + L149R + S214Q + V258P + A276S + H358D + | 58.0 |
| A364G + S388K + D470T + R483V + A499Y | |
| V45L + A79C + L149R + V258P + A276S + Q314A + A364G + | 58.0 |
| S388K | |
| V45L + A79C + L149R + V258P + A276S + L281V + A364G + | 58.0 |
| S388K | |
| V45L + A117S + L149R + S214Q + V258P + A276S + A286S + | 57.9 |
| H358D + A364G + S388K + D470T + L530R | |
| V45L + A117S + L149R + S214Q + V258P + A276S + H358D + | 57.9 |
| A364G + S388K + D470T + S501N + L530R | |
| V45L + A117S + L149R + S214Q + V258P + A276S + H358D + | 57.9 |
| A364G + S388K + D470T + N476N + L530R | |
| V45L + A117S + L149R + S214Q + V258P + A276S + H358D + | 57.9 |
| A364G + S388K + D470T + T477V + L530R | |
| V45L + A117S + L149R + S214Q + V258P + A276S + H358D + | 57.9 |
| A364G + S388H + D470T + L530R | |
| V45L + A117S + L149R + S214Q + V258P + A276S + H358D + | 57.9 |
| A364G + S388K + D470T + L478N + L530R | |
| V45L + S92D + A117S + L149R + S214Q + V258P + A276S + | 57.9 |
| H358D + A364G + S388K + D470T + L530R | |
| V45L + A117S + L149R + S214Q + V258P + A276S + H358D + | 57.9 |
| A364G + S388K + D470T + V500V + L530R | |
| V45L + A117S + L149R + S214Q + V258P + A276S + H358D + | 57.9 |
| A364G + S388K + D470T + R483V + V500V | |
| V45L + A117S + L149R + S214Q + V258P + A276S + H358D + | 57.9 |
| A364G + S388K + D470T + L478P + L530R | |
| V45L + A117S + L149R + S214Q + V258P + A276S + H358D + | 57.9 |
| A364G + S388K + D470T + R483V + S501E | |
| V45L + A117S + L149R + S214Q + V258P + A276S + H358D + | 57.9 |
| A364G + S388K + D470T + D525L + L530R | |
| V45L + A117S + L149R + S214Q + V258P + A276S + H358D + | 57.9 |
| A364G + S388K + D470T + G526T + L530R | |
| V45L + A117S + L149R + S214Q + V258P + A276S + H358D + | 57.9 |
| A364G + S388K + D470T + N527H + L530R | |
| V45L + A117S + L149R + S214Q + V258P + A276S + H358D + | 57.9 |
| A364G + S388K + D470T + T477K + L530R | |
| M28P + V45L + A117S + L149R + S214Q + V258P + A276S + | 57.9 |
| H358D + A364G + S388K + D470T + L530R | |
| D9E + V45L + A117S + L149R + S214Q + V258P + A276S + | 57.9 |
| H358D + A364G + S388K + D470T + R483V | |
| V45L + A117S + L149R + S214Q + V258P + A276S + H358D + | 57.8 |
| A364G + S388K + D470T + S501E + L530R | |
| V45L + A117S + L149R + S214Q + W250Y + V258P + A276S + | 57.8 |
| H358D + A364G + S388K + D470T + L530R | |
| V45L + A117S + L149R + S214Q + V258P + A276S + H358D + | 57.8 |
| A364G + S388K + D470T + A499L + L530R | |
| V45L + A117S + L149R + S214Q + V258P + A276S + H358D + | 57.8 |
| A364G + S388K + D470T + A472I + L530R | |
| V45L + A117S + N127D + L149R + S214Q + V258P + A276S + | 57.8 |
| H358D + A364G + S388K + D470T + R483V | |
| V45L + A117S + L149R + S214Q + V258P + A276S + H358D + | 57.7 |
| A364G + S388K + D470T + V500T + L530R | |
| V45L + A117S + L149R + S214Q + V258P + A276S + H358D + | 57.7 |
| A364G + S388K + D470T + R483V + N527H | |
| V45N + A117S + L149R + S214Q + V258P + A276S + H358D + | 57.7 |
| A364G + S388K + D470T + L530R | |
| V45L + A117S + L149R + S214Q + V258P + A276S + H358D + | 57.7 |
| A364G + S388K + D470T + T480N + L530R | |
| V45L + A117S + L149R + S214Q + V258P + A276S + H358D + | 57.7 |
| A364G + S388K + D470T + R483V + V497P | |
| V45L + A117S + L149R + S214Q + V258P + A276S + H358D + | 57.7 |
| A364G + S388K + D470T + R483V + V498I | |
| V45L + A79C + L149R + S173Y + V258P + A276S + A364G + | 57.7 |
| S388K | |
| V45L + A117S + L149R + S214Q + V258P + A276S + H358D + | 57.7 |
| A364G + S388K + D470T + T480N + R483V | |
| V45L + A117S + L149R + S214Q + H219V + V258P + A276S + | 57.6 |
| H358D + A364G + S388K + D470T + L530R | |
| V45L + A117S + L149R + S214Q + V258P + A276S + H358D + | 57.6 |
| A364G + S388A + D470T + L530R | |
| M28P + V45L + A117S + L149R + S214Q + V258P + A276S + | 57.6 |
| H358D + A364G + S388K + D470T + R483V | |
| V45L + A117S + L149R + A170S + S214Q + V258P + A276S + | 57.6 |
| H358D + A364G + S388K + D470T + R483V | |
| V45L + A79C + L149R + G187K + V258P + A276S + A364G + | 57.6 |
| S388K | |
| V45L + A117S + L149R + S214Q + V258P + A276S + H358D + | 57.6 |
| A364G + S388K + S389H + D470T + L530R | |
| V45L + D113T + A117S + L149R + S214Q + V258P + A276S + | 57.5 |
| H358D + A364G + S388K + D470T + L530R | |
| V45L + A117S + L149R + N163H + S214Q + V258P + A276S + | 57.5 |
| H358D + A364G + S388K + D470T + R483V | |
| V45L + A117S + L149R + S214Q + V258P + A276S + H358D + | 57.5 |
| A364G + S388A + D470T + R483V | |
| V45L + A117S + L149R + S214Q + V258P + A276S + H358D + | 57.5 |
| A364G + S388L + D470T + R483V | |
| V45L + Q61R + A79C + L149R + V258P + A276S + A364G + | 57.5 |
| S388K | |
| V45L + A79C + L149R + N168G + V258P + A276S + A364G + | 57.5 |
| S388K | |
| V45L + A117S + L149R + T211H + S214Q + V258P + A276S + | 57.5 |
| H358D + A364G + S388K + D470T + L530R | |
| V45L + A79C + L149R + T215V + V258P + A276S + A364G + | 57.4 |
| S388K | |
| V45L + A117S + L149R + S214Q + V258P + A276S + H358D + | 57.4 |
| A364G + S388K + D470T + R483V + V500T | |
| V45L + A79C + L149R + V258P + A276S + A286S + A364G + | 57.3 |
| S388K | |
| V45L + A79C + L149R + H219V + V258P + A276S + A364G + | 57.3 |
| S388K | |
| V45L + A79C + L149R + A170S + V258P + A276S + A364G + | 57.3 |
| S388K | |
| V45L + D113T + A117S + L149R + S214Q + V258P + A276S + | 57.3 |
| H358D + A364G + S388K + D470T + R483V | |
| V45L + Q61K + A117S + L149R + S214Q + V258P + A276S + | 57.3 |
| H358D + A364G + S388K + D470T | |
| V45L + D95N + A117S + L149R + S214Q + V258P + A276S + | 57.3 |
| H358D + A364G + S388K + G394A + D470T | |
| V45L + A79C + L149R + V258P + A276S + A364G + S388K | 57.2 |
| V45L + A117S + L149R + S214Q + V258P + A276S + A364G + | 57.2 |
| S388K + G394A + D470T + R483V | |
| V45L + A93T + A117S + L149R + S214Q + V258P + A276S + | 57.2 |
| H358D + A364G + S388K + D470T + A499Y | |
| V45L + A117S + L149R + S214Q + T215I + V258P + A276S + | 57.2 |
| H358D + A364G + S388K + D470T + R483V | |
| V45L + A117S + L149R + S214Q + H219I + V258P + A276S + | 57.2 |
| H358D + A364G + S388K + D470T + R483V | |
| V45L + A117S + L149R + S214Q + V258P + A276S + H358D + | 57.2 |
| A364G + S388K + D470T + L478N | |
| V45L + A117S + L149R + S214Q + V258P + A276S + L281V + | 57.2 |
| H358D + A364G + S388K + D470T | |
| V45L + A117S + L149R + S214E + V258P + A276S + H358D + | 57.2 |
| A364G + S388K + D470T | |
| V45L + T70R + A117S + L149R + S214Q + V258P + A276S + | 57.2 |
| L281V + H358D + A364G + S388K + G394A + C468L + | |
| D470T + R483V | |
| V45L + A117S + L149R + S214Q + V258P + A276S + H358D + | 57.2 |
| A364G + Q385C + S388K + D470T + L530R | |
| V45L + A117S + L149R + S214Q + V258P + A276S + H358D + | 57.1 |
| A364G + T366I + S388K + D470T | |
| V45L + A117S + L149R + S214Q + V258P + A276S + H358D + | 57.1 |
| A364G + S388K + D470T + S473G | |
| V45L + A117S + L149R + S214Q + V258P + A276S + H358D + | 57.1 |
| A364G + S388K + D470T + L478P | |
| V45L + A117S + L149R + S214Q + V258P + A276S + H358D + | 57.1 |
| A364G + S388K + G394D + D470T | |
| V45L + A117S + L149R + S214Q + V258P + A276S + A364G + | 57.1 |
| Q384A + S388K + D470T + R483V | |
| D11E + V45L + A79C + L149R + V258P + A276S + A364G + | 57.1 |
| S388K | |
| V45L + A117S + L149R + S214Q + V258P + A276S + H358D + | 57.1 |
| A364G + S388K + D470T + R483V + L530R | |
| V45L + A117S + L149R + S214Q + V258P + A276S + H358D + | 57.1 |
| A364G + S388K + D470T + N529T | |
| V45L + A117S + L149R + S214Q + V258P + A276S + H358D + | 57.1 |
| A364G + S388K + G394Q + D470T | |
| V45L + A117S + L149R + G187L + S214Q + V258P + A276S + | 57.1 |
| H358D + A364G + S388K + D470T | |
| V45L + A79C + L149R + V258P + A276S + A286E + A364G + | 57.0 |
| S388K | |
| V45L + A117S + L149R + S214Q + V258P + A276S + H358D + | 57.0 |
| A364G + S388K + D470T + R483V + G503L | |
| V45L + A117S + L149R + S214Q + V258P + A276S + Q314A + | 57.0 |
| H358D + A364G + S388K + D470T | |
| V45L + A117S + L149R + S214Q + V258P + A276S + V350L + | 57.0 |
| H358D + A364G + S388K + D470T | |
| V45L + A117S + L149R + G187K + S214Q + V258P + A276S + | 57.0 |
| H358D + A364G + S388K + D470T | |
| V45L + A117S + L149R + S214Q + W250Y + V258P + A276S + | 57.0 |
| H358D + A364G + S388K + D470T | |
| V45L + A117S + L149R + S214Q + V258P + A276S + A286E + | 57.0 |
| H358D + A364G + S388K + D470T | |
| V45L + A117S + L149R + S214Q + V258P + A276S + H358D + | 57.0 |
| A364G + Q384A + S388K + D470T | |
| V45L + A117S + L149R + S214Q + V258P + A276S + H358D + | 57.0 |
| A364G + S388R + D470T | |
| V45L + A117S + L149R + S214Q + V258P + A276S + H358D + | 57.0 |
| A364G + S388K + D470T + S528H | |
| V45L + A117S + L149R + S214Q + T215V + V258P + A276S + | 56.9 |
| H358D + A364G + S388K + D470T + R483V | |
| V45L + A117S + L149R + S214Q + V258P + A276S + H358D + | 56.9 |
| A364G + S388K + D470T + Q496R | |
| V45L + A117S + L149R + N163H + S214Q + V258P + A276S + | 56.9 |
| H358D + A364G + S388K + D470T | |
| V45L + A117S + L149R + S214Q + V258P + A276S + S282N + | 56.9 |
| H358D + A364G + S388K + D470T | |
| V45L + A117S + L149R + S214Q + V258P + A276Q + H358D + | 56.9 |
| A364G + S388K + D470T | |
| D11E + V45L + A117S + L149R + S214Q + V258P + A276S + | 56.9 |
| H358D + A364G + S388K + D470T | |
| V45L + A117S + L149R + S214Q + V258P + A276S + H358D + | 56.9 |
| A364G + T366V + S388K + D470T | |
| V45L + A117S + L149R + S214Q + V258P + A276S + H358D + | 56.9 |
| A364G + S388K + D470T + S473K | |
| V45L + A79C + L149R + T180Q + V258P + A276S + A364G + | 56.9 |
| S388K | |
| V45L + A117S + L149R + S214Q + V258P + A276S + H358D + | 56.9 |
| A364G + S388K + D470T + S481A + R483V | |
| V45L + A117S + L149R + S214Q + V258P + A276S + H358D + | 56.9 |
| A364G + S388R + D470T + R483V | |
| V45L + A117S + L149R + S214Q + V258P + A276S + Q314A + | 56.9 |
| A364G + S388K + D470T + R483V | |
| V45L + S92D + A117S + L149R + S214Q + V258P + A276S + | 56.9 |
| H358D + A364G + S388K + D470T | |
| V45L + A117S + L149R + S214Q + V258P + N260H + A276S + | 56.9 |
| H358D + A364G + S388K + D470T | |
| V45L + A117S + L149R + G187S + S214Q + V258P + A276S + | 56.9 |
| H358D + A364G + S388K + D470T | |
| V45L + A117S + L149R + S214Q + V258P + A276S + H358D + | 56.9 |
| A364G + S388K + D470T + T477K | |
| V45L + A117S + L149R + S214Q + V258P + A276S + H358D + | 56.9 |
| A364G + S388K + D470T + N474G | |
| V45L + A117S + L149R + S214Q + V258P + A276S + H358D + | 56.9 |
| A364G + S388K + N395D + D470T | |
| V45L + A117S + L149R + S214Q + V258P + A276S + H358D + | 56.8 |
| A364G + S388K + D470T + S501N | |
| V45L + A117S + N127D + L149R + S214Q + V258P + A276S + | 56.8 |
| H358D + A364G + S388K + D470T | |
| V45L + A117S + L149R + S214Q + V258P + A276S + H358D + | 56.8 |
| A364G + S388K + V393R + D470T | |
| V45L + A117S + L149R + S173Y + S214Q + V258P + A276S + | 56.8 |
| H358D + A364G + S388K + D470T | |
| V45L + A117S + L149R + S214Q + V258P + A276S + H358D + | 56.8 |
| A364G + S388H + D470T | |
| V45L + A117S + L149R + S214Q + T215V + V258P + A276S + | 56.8 |
| H358D + A364G + S388K + D470T + L530R | |
| V45L + A117S + L149R + G187S + S214Q + V258P + A276S + | 56.8 |
| H358D + A364G + S388K + D470T + L530R | |
| V45L + A117S + L149R + T211H + S214Q + V258P + A276S + | 56.8 |
| H358D + A364G + S388K + D470T + R483V | |
| V45L + A117S + L149R + S214Q + V258P + A276S + H358D + | 56.7 |
| A364G + S388K + D470T + T480N | |
| V45L + A117S + L149R + N168G + S214Q + V258P + A276S + | 56.7 |
| H358D + A364G + S388K + D470T | |
| V45L + A79C + L149R + H219I + V258P + A276S + A364G + | 56.7 |
| S388K | |
| V45L + L149R + S214E + V258P + A276S + H358D + A364G + | 56.7 |
| D470T | |
| V45L + Q61K + A117S + L149R + S214Q + V258P + A276S + | 56.7 |
| A364G + S388K + D470T + R483V | |
| V45L + D113T + A117S + L149R + S214Q + V258P + A276S + | 56.7 |
| H358D + A364G + S388K + D470T | |
| V45L + A117S + L149R + S214Q + V258P + A276S + A364G + | 56.7 |
| T366V + S388K + D470T + R483V | |
| V45L + A117S + L149R + S214Q + V258P + A276S + H358T + | 56.7 |
| A364G + S388K + D470T + R483V | |
| V45L + A117S + L149R + S214Q + V258P + A276S + A364G + | 56.7 |
| S388K + V393R + D470T + R483V | |
| V45N + A79C + L149R + V258P + A276S + A364G + S388K | 56.7 |
| V45L + A117S + L149R + S214Q + V258P + A276S + H358D + | 56.6 |
| A364G + S388K + D470T + Q496Q | |
| V45L + A117S + L149R + S214Q + V258P + A276S + A364G + | 56.6 |
| S388K + G394Q + D470T + R483V | |
| V45L + A79C + L149R + V258P + A276S + V350L + A364G + | 56.6 |
| S388K | |
| V45L + L149R + S214E + V258P + A276S + H358D + A364G + | 56.6 |
| D470T | |
| V45L + A117S + L149R + S214Q + V258P + A276S + A364G + | 56.6 |
| T366I + S388K + D470T + R483V | |
| V45L + A117S + L149R + S214Q + V258P + A276S + H358D + | 56.6 |
| A364G + S388K + D470T + R483V + G526T | |
| V45L + A117S + L149R + S214Q + V258P + A276S + H358D + | 56.6 |
| A364G + S388K + D470T + L484S | |
| V45L + A79C + A117S + L149R + S214Q + V258P + A276S + | 56.6 |
| H358D + A364G + S388K | |
| V45L + A117S + L149R + S214Q + V258P + A276S + H358D + | 56.6 |
| A364G + S388K + D470T + A499L | |
| V45L + A117S + L149R + S214Q + V258P + A276S + H358T + | 56.6 |
| A364G + S388K + D470T + L530R | |
| V45L + A79C + D113T + L149R + V258P + A276S + A364G + | 56.5 |
| S388K | |
| V45L + A117S + L149R + S214Q + V258P + A276S + H358D + | 56.5 |
| A364G + S388K + D470T + R483V + D525L | |
| V45L + A117S + L149R + S214Q + V258P + A276S + H358D + | 56.5 |
| A364G + S388K + D470T + V498I | |
| V45L + A117S + L149R + S214Q + V258P + A276S + H358D + | 56.5 |
| A364G + S388L + D470T | |
| V45L + T70R + A117S + L149R + S214Q + V258P + A276S + | 56.5 |
| A364G + S388K + D470T + R483V | |
| V45L + A117S + L149R + S214Q + V258P + A276S + H358D + | 56.5 |
| A364G + S388K + D470T + S528H + L530R | |
| V45L + A117S + L149R + S214Q + V258P + A276S + A364G + | 56.5 |
| S388K + D470T + N476D + R483V | |
| D9E + V45L + A117S + L149R + S214Q + V258P + A276S + | 56.5 |
| A364G + S388K + D470T + R483V | |
| V45L + A117S + L149R + S214Q + V258P + A276S + H358D + | 56.4 |
| A364G + S388K + D470T + S501E | |
| V45L + A117S + L149R + S214Q + V258P + A276S + H358D + | 56.4 |
| A364G + S388K + D470T + S481A | |
| V45L + A117S + L149R + S214Q + H219V + V258P + A276S + | 56.4 |
| H358D + A364G + S388K + D470T | |
| V45L + A117S + L149R + S214Q + V258P + A276S + H358D + | 56.4 |
| A364G + S388K + D470T + S481S | |
| V45L + A117S + L149R + G187K + S214Q + V258P + A276S + | 56.4 |
| A364G + S388K + D470T + R483V | |
| V45L + A117S + L149R + S214Q + V258P + A276S + A364G + | 56.4 |
| S388K + N395D + D470T + R483V | |
| V45L + A117S + L149R + S214Q + V258P + A276S + H358D + | 56.4 |
| A364G + S388K + I469T + D470T + R483V | |
| V45L + A79C + N127D + L149R + V258P + A276S + A364G + | 56.4 |
| S388K | |
| D9E + V45L + A79C + L149R + V258P + A276S + A364G + | 56.4 |
| S388K | |
| V45L + L149R + S214E + V258P + A276S + H358D + A364G + | 56.4 |
| D470T | |
| V45L + A117S + L149R + G187L + S214Q + V258P + A276S + | 56.4 |
| A364G + S388K + D470T + R483V | |
| V45L + A117S + L149R + S214Q + V258P + A276S + H358D + | 56.4 |
| A364G + S388K + D470T + N529T + L530R | |
| V45L + A79C + L149R + W250Y + V258P + A276S + A364G + | 56.4 |
| S388K | |
| V45L + A117S + L149R + S214Q + V258P + A276S + H358D + | 56.4 |
| A364G + S388K + D470T + V497P | |
| V45L + A117S + L149R + S214Q + V258P + A276S + H358D + | 56.4 |
| A364G + S388K + D470T + N527H | |
| V45L + A117S + L149R + S214Q + V258P + A276S + A364G + | 56.4 |
| S388K + D470T + R483V + Q496Q | |
| V45L + A117S + L149R + N168G + S214Q + V258P + A276S + | 56.4 |
| A364G + S388K + D470T + R483V | |
| V45L + A117S + L149R + S214Q + V258P + A276S + A364G + | 56.4 |
| S388K + D470T + N476N + R483V | |
| V45L + A117S + L149R + S214Q + V258P + A276S + A364G + | 56.3 |
| S388K + D470T + T477V + R483V | |
| V45L + A117S + L149R + S214Q + V258P + A276S + V350L + | 56.3 |
| A364G + S388K + D470T + R483V | |
| V45L + A117S + L149R + S214Q + V258P + A276S + A364G + | 56.3 |
| S388K + D470T + L478N + R483V | |
| V45L + A117S + L149R + S214E + V258P + A276S + A364G + | 56.3 |
| S388K + D470T + R483V | |
| M28P + V45L + A117S + L149R + S214Q + V258P + A276S + | 56.3 |
| H358D + A364G + S388K + D470T | |
| V45L + A117S + L149R + S214Q + V258P + A276S + A364G + | 56.3 |
| S388K + V393H + D470T + R483V | |
| V45L + Q61R + A117S + L149R + S214Q + V258P + A276S + | 56.3 |
| A364G + S388K + D470T + R483V | |
| V45L + A117S + L149R + S214Q + V258P + A276S + A364G + | 56.3 |
| S388K + D470T + R483V + V497P | |
| V45L + A117S + L149R + S214Q + V258P + A276S + A364G + | 56.3 |
| S388K + D470T + R483V + V498I | |
| V45L + A117S + L149R + S214Q + V258P + A276S + S282N + | 56.2 |
| H358D + A364G + S388K + D470T + L530R | |
| V45L + T70R + A117S + L149R + S214Q + V258P + A276S + | 56.2 |
| L281V + H358D + A364G + C375V + S388K + G394A + | |
| D470T + R483V | |
| V45L + A117S + L149R + S214Q + V258P + A276S + A286E + | 56.2 |
| A364G + S388K + D470T + R483V | |
| V45L + A117S + L149R + S214Q + V258P + A276S + A364G + | 56.2 |
| S388R + D470T + R483V | |
| V45L + A117S + L149R + S214Q + V258P + A276S + A364G + | 56.2 |
| S388K + D470A + R483V | |
| V45L + A117S + L149R + S214Q + V258P + A276S + A364G + | 56.2 |
| S388K + D470T + T477K + R483V | |
| V45L + A117S + L149R + S214Q + V258P + A276S + A364G + | 56.2 |
| S388K + D470T + R483V + Q496E | |
| V45L + A117S + L149R + S214Q + V258P + A276S + A364G + | 56.2 |
| S388K + D470T + R483V + Q496R | |
| V45L + A117S + L149R + S214Q + V258P + A276S + A364G + | 56.2 |
| S388K + D470T + R483V + A499L | |
| V45L + L149R + S214E + V258P + A276S + H358D + A364G + | 56.2 |
| D470T | |
| V45L + L149R + S214E + V258P + A276S + H358D + A364G + | 56.2 |
| D470T | |
| V45L + A117S + L149R + S214Q + T215I + V258P + A276S + | 56.2 |
| H358D + A364G + S388K + D470T | |
| V45L + T70R + A117S + L149R + S214Q + V258P + A276S + | 56.2 |
| L281V + H358D + A364G + S388K + G394A + C468S + | |
| D470T + R483V | |
| V45L + A117S + L149R + S214Q + V258P + A276S + A364G + | 56.1 |
| S388K + Y391V + D470T + R483V | |
| V45L + A117S + L149R + S214Q + V258P + A276Q + A364G + | 56.1 |
| S388K + D470T + R483V | |
| V45L + A117S + L149R + S214Q + V258P + A276S + H358D + | 56.1 |
| A364G + S388K + D470T + G503L + L530R | |
| V45L + A117S + L149R + S214Q + H219I + V258P + A276S + | 56.1 |
| H358D + A364G + S388K + D470T + L530R | |
| V45L + A117S + L149R + S214Q + V258P + A276S + H358D + | 56.1 |
| A364G + S388K + D470T + N476D | |
| V45L + A117S + L149R + S214Q + V258P + A276S + H358D + | 56.1 |
| A364G + S388K + V393H + D470T | |
| V45L + A117S + L149R + S214Q + V258P + A276S + A364G + | 56.1 |
| S388K + D470T + R483V | |
| V45L + A117S + L149R + S214Q + V258P + A276S + A286S + | 56.1 |
| A364G + S388K + D470T + R483V | |
| V45L + A117S + L149R + S214Q + V258P + A276S + A364G + | 56.1 |
| S388K + D470T + R483V + S501E | |
| V45L + A117S + L149R + S214Q + V258P + A276S + A364G + | 56.1 |
| S388K + D470T + R483V + N529T | |
| V45L + A117S + L149R + S214Q + V258P + A276S + A364G + | 56.1 |
| S388K + D470T + R483V + S501N | |
| V45L + A117S + L149R + S214Q + V258P + A276S + H358D + | 56.1 |
| A364G + S388K + D470T | |
| V45L + A117S + L149R + S214Q + V258P + A276S + A364G + | 56.1 |
| S388K + D470T + S481S + R483V | |
| V45L + A117S + L149R + S214Q + V258P + A276S + A364G + | 56.1 |
| S388K + D470T + L478P + R483V | |
| D11E + V45L + A117S + L149R + S214Q + V258P + A276S + | 56.1 |
| A364G + S388K + D470T + R483V | |
| V45L + A117S + L149R + S214Q + V258P + A276S + H358D + | 56.0 |
| A364G + S388K + D470T + T477V | |
| V45L + A117S + L149R + S214Q + V258P + A276S + H358D + | 56.0 |
| A364G + S388K + D470T + V497P + L530R | |
| V45L + A117S + L149R + T211H + S214Q + V258P + A276S + | 56.0 |
| H358D + A364G + S388K + D470T | |
| V45L + A117S + L149R + S214Y + V258P + A276S + H358D + | 56.0 |
| A364G + S388K + D470T + R483V | |
| V45L + A117S + L149R + S214Q + W250Y + V258P + A276S + | 56.0 |
| A364G + S388K + D470T + R483V | |
| V45L + S92D + A117S + L149R + S214Q + V258P + A276S + | 56.0 |
| A364G + S388K + D470T + R483V | |
| V45L + A117S + L149R + S214Q + V258P + A276S + A364G + | 56.0 |
| S388K + D470T + R483V + N527H | |
| V45N + A117S + L149R + S214Q + V258P + A276S + A364G + | 56.0 |
| S388K + D470T + R483V | |
| V45L + A117S + L149R + S214Q + V258P + A276S + H358D + | 55.9 |
| A364G + Q385C + S388K + D470T | |
| V45L + A117S + L149R + S173Y + S214Q + V258P + A276S + | 55.9 |
| A364G + S388K + D470T + R483V | |
| V45L + A117S + L149R + S214Q + V258P + A276S + A364G + | 55.9 |
| S388H + D470T + R483V | |
| V45L + A117S + L149R + S214Q + V258P + A276S + A364G + | 55.9 |
| S388K + S389H + D470T + R483V | |
| V45L + A117S + L149R + S214Q + H219V + V258P + A276S + | 55.9 |
| A364G + S388K + D470T + R483V | |
| V45L + A79C + A117S + L149R + S214Q + V258P + A276S + | 55.9 |
| A364G + S388K + D470T | |
| V45L + A117S + L149R + S214Q + T215I + V258P + A276S + | 55.9 |
| H358D + A364G + S388K + D470T + L530R | |
| V45L + A117S + L149R + S214Q + V258P + A276S + H358D + | 55.9 |
| A364G + S388K + D470T + Q496E | |
| V45L + A117S + L149R + A170S + S214Q + V258P + A276S + | 55.9 |
| H358D + A364G + S388K + D470T | |
| V45L + A117S + L149R + S214Q + V258P + A276S + H358D + | 55.8 |
| A364G + S388K + S389V + D470T + R483V | |
| V45L + A117S + L149R + S214Q + V258P + A276S + H358D + | 55.8 |
| A364G + S388K + S389V + D470T + L530R | |
| V45L + A117S + L149R + S214Q + V258P + A276S + H358D + | 55.8 |
| A364G + S388A + D470T | |
| V45L + A117S + L149R + S214Q + V258P + A276S + H358D + | 55.8 |
| A364G + S388K + S389V + D470T | |
| V45L + T70R + A117S + L149R + S214Q + V258P + A276S + | 55.8 |
| H358D + A364G + S388K + D470T | |
| V45L + A117S + L149R + S214Q + H219I + V258P + A276S + | 55.8 |
| A364G + S388K + D470T + R483V | |
| V45L + A79C + L149R + N163H + V258P + A276S + A364G + | 55.7 |
| S388K | |
| V45L + A117S + L149R + S214Q + V258P + A276S + A364G + | 55.7 |
| S388K + D470T + R483V + A499Y | |
| V45L + A117S + L149R + S214Q + V258P + A276S + L281V + | 55.7 |
| A364G + S388K + D470T + R483V | |
| V45L + A117S + L149R + S214Q + V258P + A276S + A364G + | 55.6 |
| S388K + D470T + R483V + V500T | |
| V45L + A117S + L149R + T180Q + S214Q + V258P + A276S + | 55.6 |
| H358D + A364G + S388K + D470T | |
| V45L + A117S + L149R + S214Q + V258P + A276S + H358D + | 55.6 |
| A364G + S388K + D470T + V500V | |
| V45L + A117S + L149R + S214Q + V258P + A276S + H358D + | 55.6 |
| A364G + S388K + D470T + G526T | |
| V45L + C60L + T70R + A117S + L149R + S214Q + V258P + | 55.6 |
| A276S + L281V + H358D + A364G + S388K + G394A + | |
| D470T + R483V | |
| V45L + C69L + T70R + A117S + L149R + S214Q + V258P + | 55.6 |
| A276S + L281V + H358D + A364G + S388K + G394A + | |
| D470T + R483V | |
| V45L + A117S + L149R + S214Q + V258P + A276S + A364G + | 55.5 |
| S388A + D470T + R483V | |
| V45L + A117S + L149R + S214Q + V258P + A276S + A286S + | 55.5 |
| H358D + A364G + S388K + D470T | |
| V45L + A117S + L149R + S214Q + V258P + A276S + H358D + | 55.4 |
| A364G + S388K + D470T + R483V + V497D | |
| V45L + A117S + L149R + S214Q + V258P + A276S + H358D + | 55.4 |
| A364G + S388K + D470A + L530R | |
| V45L + A117S + L149R + S214Q + V258P + A276S + A364G + | 55.4 |
| S388K + D470T + R483V + D525L | |
| V45L + A117S + L149R + S214Q + V258P + A276S + H358D + | 55.4 |
| A364G + S388K + D470T + N476N | |
| V45L + A117S + L149R + S214Q + V258P + A276S + L281V + | 55.3 |
| A364G + S388K + D470T | |
| V45L + A117S + L149R + S214Q + V258P + A276S + A364G + | 55.3 |
| Q384A + S388K + D470T | |
| V45L + A117S + L149R + S214Q + V258P + A276S + A286E + | 55.3 |
| A364G + S388K + D470T | |
| V45L + A117S + L149R + S214Q + V258P + A276S + A364G + | 55.2 |
| S388K + D470T + T477K | |
| V45L + A117S + L149R + S214Q + V258P + A276S + H358D + | 55.1 |
| A364G + S388K + S389H + D470T | |
| V45L + A117S + L149R + S214Y + V258P + A276S + H358D + | 55.1 |
| A364G + S388K + D470T + L530R | |
| V45L + A117S + L149R + S214Y + V258P + A276S + H358D + | 55.1 |
| A364G + S388K + D470T | |
| V45L + A117S + L149R + S214Q + V258P + A276S + A364G + | 55.1 |
| S388K + G394A + D470T | |
| V45L + A117S + L149R + S214Q + V258P + A276S + A364G + | 55.1 |
| S388K + D470T + N476D | |
| V45L + A117S + L149R + S214Q + V258P + A276S + H358D + | 55.0 |
| A364G + S388K + D470T + L478N + R483V | |
| V45L + C60A + T70R + A117S + L149R + S214Q + V258P + | 55.0 |
| A276S + L281V + H358D + A364G + S388K + G394A + | |
| D470T + R483V | |
| V45L + A117S + L149R + S214Q + V258P + A276S + A364G + | 55.0 |
| S388K + V393H + D470T | |
| V45L + A117S + L149R + S214Q + V258P + A276S + A364G + | 55.0 |
| S388K + N395D + D470T | |
| A25W + V45L + A117S + L149R + S214Q + V258P + A276S + | 55.0 |
| H358D + A364G + S388K + D470T | |
| V45L + T70R + A117S + L149R + S214Q + C243L + V258P + | 55.0 |
| A276S + L281V + H358D + A364G + S388K + G394A + | |
| D470T + R483V | |
| V45L + A117S + L149R + S214Q + V258P + A276S + H358T + | 55.0 |
| A364G + S388K + D470T | |
| V45L + A117S + L149R + S214Q + V258P + A276S + A364G + | 54.9 |
| S388K + D470T + L478N | |
| V45L + C60S + T70R + A117S + L149R + S214Q + V258P + | 54.9 |
| A276S + L281V + H358D + A364G + S388K + G394A + | |
| D470T + R483V | |
| V45L + A117S + L149R + S214Q + W250Y + V258P + A276S + | 54.9 |
| A364G + S388K + D470T | |
| V45L + A117S + L149R + S214Q + T215V + V258P + A276S + | 54.9 |
| A364G + S388K + D470T + R483V | |
| V45L + Q61R + A117S + L149R + S214Q + V258P + A276S + | 54.8 |
| H358D + A364G + S388K + D470T | |
| V45L + A117S + L149R + G187L + S214Q + V258P + A276S + | 54.8 |
| A364G + S388K + D470T | |
| D9E + V45L + A117S + L149R + S214Q + V258P + A276S + | 54.8 |
| A364G + S388K + D470T | |
| V45L + A117S + L149R + S214Q + V258P + A276S + H358D + | 54.8 |
| A364G | |
| V45L + C69A + T70R + A117S + L149R + S214Q + V258P + | 54.8 |
| A276S + L281V + H358D + A364G + S388K + G394A + | |
| D470T + R483V | |
| V45L + T70R + A117S + L149R + C161S + S214Q + V258P + | 54.8 |
| A276S + L281V + H358D + A364G + S388K + G394A + | |
| D470T + R483V | |
| V45L + A117S + L149R + S214Q + H219I + V258P + A276S + | 54.8 |
| H358D + A364G + S388K + D470T | |
| V45L + A117S + L149R + S214Q + V258P + A276S + S282N + | 54.8 |
| A364G + S388K + D470T | |
| V45L + A117S + L149R + T211H + S214Q + V258P + A276S + | 54.7 |
| A364G + S388K + D470T + R483V | |
| V45L + D95N + A117S + L149R + S214Q + V258P + A276S + | 54.7 |
| H358D + A364G + S388K + S473G | |
| V45L + T70R + D95N + A117S + L149R + S214Q + V258P + | 54.7 |
| A276S + L356M + H358D + A364G + S388K | |
| D11E + V45L + A117S + L149R + S214Q + V258P + A276S + | 54.7 |
| A364G + S388K + D470T | |
| V45L + A117S + L149R + S214Q + V258P + A276S + A364G + | 54.7 |
| S388K + D470T + N476N | |
| V45L + A117S + L149R + S214Q + V258P + A276S + A364G + | 54.7 |
| S388K + G394D + D470T | |
| V45L + Q61K + A117S + L149R + S214Q + V258P + A276S + | 54.7 |
| A364G + S388K + D470T | |
| V45L + A117S + L149R + S214Q + V258P + A276S + H358D + | 54.7 |
| A364G + S388K + D470T + G503L | |
| V45L + A117S + L149R + S214Q + V258P + A276S + A286S + | 54.7 |
| A364G + S388K + D470T | |
| V45L + A117S + L149R + S214Q + V258P + A276S + H358D + | 54.6 |
| A364G + S388K + D470A | |
| V45L + A117S + L149R + S214Q + V258P + A276S + A364G + | 54.6 |
| S388K + G394Q + D470T | |
| V45L + A117S + L149R + S214Q + V258P + A276S + A364G + | 54.6 |
| S388R + D470T | |
| V45L + A117S + L149R + S214Q + V258P + A276S + A364G + | 54.6 |
| S388K + D470T + L478P | |
| V45L + A117S + L149R + S214Q + V258P + A276S + V350L + | 54.6 |
| A364G + S388K + D470T | |
| V45L + D113T + A117S + L149R + S214Q + V258P + A276S + | 54.6 |
| A364G + S388K + D470T | |
| V45L + A117S + L149R + T180Q + S214Q + V258P + A276S + | 54.6 |
| A364G + S388K + D470T | |
| V45L + C69V + T70R + A117S + L149R + S214Q + V258P + | 54.6 |
| A276S + L281V + H358D + A364G + S388K + G394A + | |
| D470T + R483V | |
| V45L + A117S + L149R + S214Q + V258P + A276S + H358D + | 54.6 |
| A364G + S388K + D470T + D525L | |
| V45L + A117S + L149R + S214Q + V258P + A276S + A364G + | 54.5 |
| S388K + D470T | |
| V45L + A117S + L149R + S214Q + V258P + A276S + H358D + | 54.5 |
| A364G + S388K + D470T + V500T | |
| V45L + A117S + L149R + S214Q + V258P + A276S + A364G + | 54.4 |
| S388K + S389H + D470T | |
| V45L + A117S + L149R + S214Q + V258P + A276S + A364G + | 54.4 |
| S388K + V393R + D470T | |
| V45L + A117S + L149R + S214Q + V258P + A276S + A364G + | 54.4 |
| S388K + D470T + Q496R | |
| V45L + A117S + N127D + L149R + S214Q + V258P + A276S + | 54.4 |
| A364G + S388K + D470T | |
| V45L + A117S + L149R + S214Q + V258P + A276S + A364G + | 54.4 |
| S388K + D470T + A499Y | |
| V45L + A117S + L149R + S214Q + T215V + V258P + A276S + | 54.4 |
| H358D + A364G + S388K + D470T | |
| V45L + A117S + L149R + S214Q + V258P + A276S + H358D + | 54.4 |
| A364G + S388K + D470T + T480L | |
| V45L + A117S + L149R + S214Q + V258P + A276S + A364G + | 54.4 |
| S388L + D470T | |
| V45L + A117S + L149R + S173Y + S214Q + V258P + A276S + | 54.4 |
| A364G + S388K + D470T | |
| V45L + S92D + A117S + L149R + S214Q + V258P + A276S + | 54.4 |
| A364G + S388K + D470T | |
| V45L + A117S + L149R + S214Q + V258P + A276S + A364G + | 54.4 |
| S388K + S389V + D470T + R483V | |
| V45L + A117S + L149R + S214Q + V258P + A276S + A364G + | 54.4 |
| S388K + D470T + N529T | |
| V45L + C60V + T70R + A117S + L149R + S214Q + V258P + | 54.3 |
| A276S + L281V + H358D + A364G + S388K + G394A + | |
| D470T + R483V | |
| V45L + A117S + L149R + S214Q + V258P + A276S + A364G + | 54.3 |
| S388K + D470T + R483V + V497E | |
| V45L + A117S + L149R + S214Q + V258P + A276S + H358D + | 54.3 |
| A364G + S388K + L478N | |
| V45L + T70R + A117S + L149R + S214Q + V258P + A276S + | 54.3 |
| A364G + S388K + D470T | |
| M28P + V45L + A117S + L149R + S214Q + V258P + A276S + | 54.3 |
| A364G + S388K + D470T | |
| V45L + A117S + L149R + S214Q + V258P + A276Q + A364G + | 54.3 |
| S388K + D470T | |
| V45L + A117S + L149R + S214Q + V258P + A276S + H358D + | 54.3 |
| A364G + S388K + D470V + R483V | |
| V45L + A117S + L149R + S214Y + V258P + A276S + A364G + | 54.3 |
| S388K + D470T + R483V | |
| V45L + A117S + L149R + S214Q + V258P + A276S + H358D + | 54.3 |
| A364G + S388K + N395D | |
| V45L + A117S + L149R + S214Q + V258P + A276S + H358D + | 54.3 |
| A364G + S388K + T477K | |
| V45L + A117S + L149R + S214Q + V258P + A276S + L281V + | 54.3 |
| H358D + A364G + S388K | |
| V45L + Q61R + A117S + L149R + S214Q + V258P + A276S + | 54.2 |
| A364G + S388K + D470T | |
| V45L + A117S + L149R + S214Q + T215I + V258P + A276S + | 54.2 |
| A364G + S388K + D470T | |
| V45L + A117S + L149R + S214Q + V258P + A276S + A364G + | 54.2 |
| S388K + D470T + Q496E | |
| V45L + A117S + L149R + G187K + S214Q + V258P + A276S + | 54.2 |
| A364G + S388K + D470T | |
| V45L + A117S + L149R + S214Q + V258P + A276S + A364G + | 54.2 |
| S388K + D470T + W495I | |
| V45L + A117S + L149R + S214Q + V258P + A276S + A364G + | 54.2 |
| S388K + D470T + V498I | |
| V45L + A117S + L149R + S214Q + V258P + A276S + H358D + | 54.2 |
| A364G + S388K + D470K + R483V | |
| V45L + A117S + L149R + S214Q + V258P + A276S + A364G + | 54.2 |
| S388K + D470T + V500V | |
| D11E + V45L + A117S + L149R + S214Q + V258P + A276S + | 54.2 |
| H358D + A364G + S388K | |
| V45L + A117S + L149R + A170S + S214Q + V258P + A276S + | 54.2 |
| A364G + S388K + D470T | |
| V45L + A117S + L149R + S214Q + V258P + A276S + A364G + | 54.2 |
| S388K + Y391V + D470T | |
| V45L + A117S + L149R + S214Q + V258P + A276S + Q314A + | 54.2 |
| A364G + S388K + D470T | |
| V45L + A117S + L149R + S214Q + V258P + A276S + A364G + | 54.2 |
| S388K + D470T + T477V | |
| A25W + V45L + A79C + L149R + S214E + V258P + A276S + | 54.2 |
| A364G | |
| V45L + A117S + L149R + S214Q + V258P + A276S + H358D + | 54.1 |
| A364G + S388K + G394Q | |
| V45L + A117S + L149R + S214Q + H219V + V258P + A276S + | 54.1 |
| A364G + S388K + D470T | |
| V45L + A117S + L149R + S214Q + V258P + A276S + A364G + | 54.1 |
| S388K + D470T + Q496Q | |
| V45L + T70R + A117S + L149R + S214Q + V258P + A276S + | 54.1 |
| L281V + H358D + A364G + C375A + S388K + G394A + | |
| D470T + R483V | |
| V45L + A117S + L149R + D192E + S214Q + V258P + A276S + | 54.1 |
| H358D + A364G | |
| V45L + A117S + L149R + S214Q + V258P + A276S + H358D + | 54.1 |
| A364G + S388K + D470T + A472L | |
| V45L + A117S + L149R + S214Q + V258P + A276S + H358D + | 54.1 |
| A364G + S388K + A499Y | |
| V45L + A117S + L149R + S214Q + T215V + V258P + A276S + | 54.0 |
| A364G + S388K + D470T | |
| V45L + A117S + L149R + S214Q + V258P + A276S + A364G + | 54.0 |
| Q385C + S388K + D470T | |
| V45L + A117S + L149R + S214Q + V258P + A276S + A364G + | 54.0 |
| S388A + D470T | |
| V45L + A117S + L149R + S214Q + V258P + A276S + A364G + | 54.0 |
| S388H + D470T | |
| V45L + A117S + L149R + S214Q + V258P + A276S + A364G + | 54.0 |
| S388K + D470T + T480N | |
| V45L + A117S + L149R + S214Q + V258P + A276S + A364G + | 54.0 |
| S388K + D470T + V497P | |
| V45L + A117S + L149R + S214Q + V258P + A276S + A364G + | 54.0 |
| S388K + D470T + A499L | |
| V45L + A117S + L149R + S214Q + V258P + A276S + A364G + | 54.0 |
| S388K + D470T + S501N | |
| V45L + A117S + L149R + S214Q + V258P + A276S + A364G + | 54.0 |
| D470T | |
| V45L + A117S + L149R + S214Q + V258P + A276S + H358D + | 54.0 |
| A364G + Q384A + S388K | |
| V45L + T70R + A117S + L149R + S214Q + V258P + A276S + | 54.0 |
| L281V + H358D + A364G + C375S + S388K + G394A + | |
| D470T + R483V | |
| V45L + A117S + L149R + S214Q + V258P + A276S + A364G + | 53.9 |
| S388L + D470T + R483V | |
| V45L + A79C + S92D + L149R + V258P + A276S + A364G + | 53.9 |
| S388K | |
| V45L + T70R + A117S + L149R + S214Q + V258P + A276S + | 53.9 |
| L281V + H358D + A364G + S388K + G394A + D470T + | |
| R483V + C540A | |
| V45N + A117S + L149R + S214Q + V258P + A276S + A364G + | 53.9 |
| S388K + D470T | |
| V45L + A117S + L149R + S214Q + V258P + A276S + H358D + | 53.9 |
| A364G + S388K + G394A | |
| V45L + C69S + T70R + A117S + L149R + S214Q + V258P + | 53.8 |
| A276S + L281V + H358D + A364G + S388K + G394A + | |
| D470T + R483V | |
| V45L + A117S + L149R + S214Q + V258P + A276S + H358D + | 53.8 |
| A364G + S388K + V498I | |
| V45L + A117S + L149R + S214Q + V258P + A276S + H358D + | 53.8 |
| A364G | |
| V45L + A117S + L149R + S214Q + H219I + V258P + A276S + | 53.8 |
| A364G + S388K + D470T | |
| V45L + A117S + L149R + N163H + S214Q + V258P + A276S + | 53.7 |
| A364G + S388K + D470T | |
| V45L + L149R + S214Q + V258P + A276S + H358D + A364G | 53.7 |
| V45L + A117S + L149R + S214Q + V258P + A276S + Q314A + | 53.7 |
| H358D + A364G + S388K | |
| V45L + A117S + L149R + S173Y + S214Q + V258P + A276S + | 53.7 |
| H358D + A364G + S388K | |
| V45L + A117S + L149R + S214E + V258P + A276S + H358D + | 53.7 |
| A364G + S388K | |
| V45L + A117S + L149R + S214Q + V258P + A276S + H358D + | 53.7 |
| A364G + S388K + G394D | |
| A25W + V45L + A79C + L149R + V258P + A276S + A364G | 53.6 |
| V45L + L149R + S214Q + V258P + A276S + A286E + A364G + | 53.6 |
| S388K | |
| V45L + Q61R + A117S + L149R + S214Q + V258P + A276S + | 53.6 |
| H358D + A364G + S388K | |
| V45L + A117S + L149R + S214Q + V258P + A276S + H358D + | 53.6 |
| A364G + T366V + S388K | |
| V45L + A117S + L149R + D192E + S214Q + V258P + A276S + | 53.6 |
| H358D + A364G | |
| V45L + A117S + L149R + T180Q + S214Q + V258P + A276S + | 53.5 |
| H358D + A364G + S388K | |
| V45L + A117S + L149R + S214Q + T215I + V258P + A276S + | 53.5 |
| H358D + A364G + S388K | |
| V45L + A117S + L149R + S214Q + W250Y + V258P + A276S + | 53.5 |
| H358D + A364G + S388K | |
| V45L + L149R + S214Q + T215I + V258P + A276S + A364G + | 53.5 |
| S388K | |
| V45L + A117S + L149R + S214Q + V258P + A276S + H358D + | 53.5 |
| A364G + S388K + N476D | |
| V45L + D113T + A117S + L149R + S214Q + V258P + A276S + | 53.5 |
| A364G + S388K + D470T + R483V | |
| V45L + A117S + L149R + S214Q + T215K + V258P + A276S + | 53.4 |
| A364G + S388K + D470T + R483V | |
| V45L + A117S + L149R + S214Q + V258P + A276S + H358D + | 53.4 |
| A364G + S388K | |
| V45L + A117S + L149R + S214Q + V258P + A276S + H358D + | 53.4 |
| A364G + S388K + T477V | |
| V45L + A117S + L149R + S214Q + V258P + A276S + H358D + | 53.4 |
| A364G + S388K + L478P | |
| V45L + A117S + L149R + S214Q + V258P + A276S + H358D + | 53.4 |
| A364G + S388K + S481A | |
| A25W + V45L + L149R + S214Q + V258P + A276S + A364G + | 53.4 |
| S388K | |
| V45L + A117S + L149R + T211H + S214Q + V258P + A276S + | 53.4 |
| A364G + S388K + D470T | |
| V45L + A117S + L149R + S214Q + T215K + V258P + A276S + | 53.4 |
| H358D + A364G + S388K + D470T + R483V | |
| V45L + A79C + L149R + V258P + A276S + L281V + A364G | 53.4 |
| V45L + A117S + L149R + S214Q + V258P + A276S + A364G + | 53.4 |
| S388K + R483V | |
| V45L + A117S + L149R + S214Q + V258P + A276S + H358D + | 53.4 |
| A364G + S388K + V500T | |
| V45L + A117S + L149R + S214Q + V258P + A276S + V350L + | 53.3 |
| H358D + A364G + S388K | |
| V45L + A117S + L149R + G187K + S214Q + V258P + A276S + | 53.3 |
| H358D + A364G + S388K | |
| V45L + T70R + A117S + L149R + S214Q + V258P + A276S + | 53.3 |
| L281V + H358D + A364G + C375L + S388K + G394A + | |
| D470T + R483V | |
| V45L + L149R + A170S + S214Q + V258P + A276S + A364G + | 53.3 |
| S388K | |
| V45L + A117S + L149R + S214E + V258P + A276S + A364G + | 53.3 |
| S388K + D470T | |
| V45L + A117S + L149R + S214Q + V258P + A276S + A364G + | 53.3 |
| S388K + D470V + R483V | |
| V45L + A117S + L149R + S214Q + V258P + A276S + H358D + | 53.3 |
| A364G + S388K + V500V | |
| V45L + Q61K + L149R + S214Q + V258P + A276S + A364G + | 53.3 |
| S388K | |
| V45L + L149R + N168G + S214Q + V258P + A276S + A364G + | 53.3 |
| S388K | |
| V45L + A117S + L149R + N163H + S214Q + V258P + A276S + | 53.3 |
| H358D + A364G + S388K | |
| V45L + A79C + D95D + L149R + V258P + A276S + A364G + | 53.3 |
| S388K | |
| V45L + Q77E + A79C + L149R + V258P + A276S + A364G + | 53.3 |
| S388K | |
| V45L + A117S + L149R + S214Q + V258P + A276S + A364G + | 53.2 |
| S388K + D470T + R483V + V497W | |
| V45L + L149R + S214Q + V258P + A276S + S282N + A364G + | 53.2 |
| S388K | |
| V45L + A117S + L149R + S214Q + V258P + A276S + H358D + | 53.2 |
| A364G + S388R | |
| V45L + A117S + L149R + S214Q + V258P + A276S + H358D + | 53.2 |
| A364G + S388K + S481S | |
| V45L + A117S + L149R + S214Q + V258P + A276S + A364G + | 53.2 |
| S388K + D470T + V500T | |
| V45L + T70R + A117S + L149R + S214Q + V258P + A276S + | 53.2 |
| L281V + H358D + A364G + S388K + G394A + D470T + | |
| R483V + C540L | |
| V45L + T70R + A117S + L149R + S214Q + V258P + A276S + | 53.2 |
| L281V + H358D + A364G + S388K + G394A + D470T + | |
| R483V + C522S | |
| V45L + A117S + L149R + S214Q + V258P + A276S + H358D + | 53.2 |
| A364G + S388K + W495S | |
| V45L + L149R + S214Q + V258P + A276S + Q314A + A364G + | 53.2 |
| S388K | |
| V45L + D113T + A117S + L149R + S214Q + V258P + A276S + | 53.2 |
| H358D + A364G + S388K | |
| V45L + A117S + L149R + S214Q + V258P + A276S + H358D + | 53.1 |
| A364G + S388K + D470T + R483V + V497W | |
| V45L + T70R + A117S + L149R + S214Q + V258P + A276S + | 53.1 |
| L281V + H358D + A364G + S388K + G394A + D470T + | |
| R483V + C540S | |
| V45L + T70R + A117S + L149R + S214Q + V258P + A276S + | 53.1 |
| L281V + H358D + A364G + S388K + G394A + C396S + | |
| D470T + R483V | |
| V45L + T70R + L149R + S214Q + V258P + A276S + A364G + | 53.1 |
| S388K | |
| V45L + A117S + L149R + S214Q + V258P + A276S + H358D + | 53.1 |
| A364G + S388K + N474G | |
| V45L + A117S + L149R + S214Q + V258P + A276S + S282N + | 53.1 |
| H358D + A364G + S388K | |
| V45L + A117S + L149R + S214Q + V258P + A276S + H358D + | 53.1 |
| A364G + S388K + Q496Q | |
| V45L + A117S + L149R + S214Q + V258P + A276S + A364G + | 53.1 |
| S388K + S389V + D470T | |
| V45L + T70R + A117S + L149R + S214Q + V258P + A276S + | 53.0 |
| L281V + H358D + A364G + S388K + G394A + C396A + | |
| D470T + R483V | |
| V45L + A117S + L149R + S214Q + V258P + A276S + H358D + | 53.0 |
| A364G + S388K + S501E | |
| A25W + V45L + L149R + S214E + V258P + A276S + A364G | 53.0 |
| V45L + L149R + S214Q + W250Y + V258P + A276S + A364G + | 53.0 |
| S388K | |
| V45L + L149R + G187K + S214Q + V258P + A276S + A364G + | 53.0 |
| S388K | |
| V45L + A117S + L149R + S214Q + V258P + A276S + H358D + | 53.0 |
| A364G + S388K + N476N | |
| V45L + A117S + L149R + S214Q + V258P + A276S + H358D + | 53.0 |
| A364G + S388K + S473K | |
| V45L + A117S + L149R + G187L + S214Q + V258P + A276S + | 53.0 |
| H358D + A364G + S388K | |
| C40L + V45L + T70R + A117S + L149R + S214Q + V258P + | 53.0 |
| A276S + L281V + H358D + A364G + S388K + G394A + | |
| D470T + R483V | |
| V45L + A117S + L149R + S214Q + V258P + A276S + H358D + | 53.0 |
| A364G + S388K + Q496E | |
| A25W + V45L + A117S + L149R + S214Q + V258P + A276S + | 53.0 |
| H358D + A364G + S388K | |
| V45L + L149R + V258P + A276S + L281V + H358T + A364G + | 53.0 |
| S388K | |
| V45L + A117S + L149R + S214Q + V258P + A276S + H358D + | 53.0 |
| A364G + S388A | |
| V45L + A117S + L149R + N168G + S214Q + V258P + A276S + | 53.0 |
| H358D + A364G + S388K | |
| V45L + A117S + L149R + S214Q + V258P + A276S + H358D + | 53.0 |
| A364G + S388K + S389H | |
| M28P + V45L + L149R + S214Q + V258P + A276S + A364G + | 53.0 |
| S388K | |
| V45L + A117S + L149R + S214Q + V258P + A276S + A364G + | 52.9 |
| S388K + D470T + T480L | |
| V45L + A117S + L149R + S214Q + V258P + A276S + A364G + | 52.9 |
| Q384A + S388K | |
| V45L + A93S + L149R + A252S + H334D | 52.9 |
| C40A + V45L + T70R + A117S + L149R + S214Q + V258P + | 52.9 |
| A276S + L281V + H358D + A364G + S388K + G394A + | |
| D470T + R483V | |
| V45L + L149R + G187L + S214Q + V258P + A276S + A364G + | 52.9 |
| S388K | |
| V45L + A117S + L149R + S214Q + V258P + A276S + H358D + | 52.9 |
| A364G + Q385C + S388K | |
| V45L + L149R + S214Q + V258P + A276S + A286S + A364G + | 52.9 |
| S388K | |
| V45L + S92D + A117S + L149R + S214Q + V258P + A276S + | 52.9 |
| H358D + A364G + S388K | |
| V45L + A117S + L149R + S214Q + V258P + A276S + H358D + | 52.9 |
| A364G + S388L | |
| V45L + A117S + L149R + S214Q + V258P + A276S + A286E + | 52.8 |
| A364G + S388K | |
| D9E + V45L + L149R + S214Q + V258P + A276S + A364G + | 52.8 |
| S388K | |
| V45L + L149R + S173Y + S214Q + V258P + A276S + A364G + | 52.8 |
| S388K | |
| V45L + A117S + L149R + A170S + S214Q + V258P + A276S + | 52.8 |
| H358D + A364G + S388K | |
| V45L + A117S + L149R + S214Q + V258P + A276S + H358D + | 52.8 |
| A364G + S388K + Y391V | |
| V45L + A117S + L149R + S214Q + V258P + A276S + L281V + | 52.8 |
| A364G + S388K | |
| V45L + A117S + L149R + S214Q + V258P + A276S + A364G + | 52.8 |
| S388K + G394D | |
| V45L + A117S + L149R + S214Q + V258P + A276S + A364G + | 52.8 |
| S388K + S473G | |
| V45L + A79C + L149R + V258P + A276S + H358T + A364G + | 52.8 |
| S388K | |
| V45L + L149R + S214Q + V258P + A276Q + A364G + S388K | 52.8 |
| A25W + V45L + L149R + S214E + V258P + A276S + H358T + | 52.7 |
| A364G | |
| V45L + A117S + L149R + S214Q + V258P + A276S + H358D + | 52.7 |
| A364G + S388K + T480N | |
| D9E + V45L + A117S + L149R + S214Q + V258P + A276S + | 52.7 |
| H358D + A364G + S388K | |
| V45L + A117S + L149R + S214Q + H219I + V258P + A276S + | 52.7 |
| H358D + A364G + S388K | |
| C40S + V45L + T70R + A117S + L149R + S214Q + V258P + | 52.7 |
| A276S + L281V + H358D + A364G + S388K + G394A + | |
| D470T + R483V | |
| C40V + V45L + T70R + A117S + L149R + S214Q + V258P + | 52.7 |
| A276S + L281V + H358D + A364G + S388K + G394A + | |
| D470T + R483V | |
| A25W + V45L + L149R + S214E + V258P + A276S + A286E + | 52.7 |
| A364G | |
| V45L + A117S + L149R + S214Q + V258P + A276S + A364G + | 52.7 |
| S388K + G394A | |
| V45L + A117S + L149R + S214Q + V258P + A276S + H358D + | 52.7 |
| A364G + S388K + A499L | |
| A25W + V45L + Q61K + L149R + S214E + V258P + A276S + | 52.6 |
| A364G | |
| V45L + A117S + L149R + S214Q + T215V + V258P + A276S + | 52.6 |
| H358D + A364G + S388K | |
| A25W + V45L + L149R + V258P + A276S + H358T + A364G + | 52.6 |
| S388K | |
| V45L + A117S + L149R + S214Q + V258P + A276S + A364G + | 52.6 |
| S388K + D470T + R483V + W495E | |
| A25W + V45L + A79C + L149R + V258P + A276S | 52.6 |
| V45L + L149R + S214Q + V258P + A276S + V350L + A364G + | 52.6 |
| S388K | |
| D11E + V45L + L149R + S214Q + V258P + A276S + A364G + | 52.6 |
| S388K | |
| V45L + L149R + H219I + V258P + A276S + H358T + A364G + | 52.5 |
| S388K | |
| V45L + T70R + A117S + L149R + S214Q + V258P + A276S + | 52.5 |
| L281V + H358D + A364G + S388K + G394A + C396V + | |
| D470T + R483V | |
| V45N + L149R + S214Q + V258P + A276S + A364G + S388K | 52.5 |
| V45L + A117S + L149R + S214Q + H219V + V258P + A276S + | 52.5 |
| H358D + A364G + S388K | |
| V45N + A117S + L149R + S214Q + V258P + A276S + H358D + | 52.5 |
| A364G + S388K | |
| V45L + S92D + L149R + S214Q + V258P + A276S + A364G + | 52.5 |
| S388K | |
| V45L + L149R + V258P + A276S + H358T + A364G | 52.5 |
| V45L + A117S + L149R + S214Q + V258P + A276S + A364G + | 52.5 |
| S388K + L478P | |
| V45L + A117S + L149R + S214Q + V258P + A276S + A364G + | 52.5 |
| S388K + S481A | |
| V45L + A117S + L149R + S214Q + V258P + A276S + Q314A + | 52.5 |
| A364G + S388K | |
| A25W + V45L + L149R + T211H + S214E + V258P + A276S + | 52.4 |
| A364G | |
| A25W + V45L + L149R + S214E + H219V + V258P + A276S + | 52.4 |
| A364G | |
| V45L + A117S + L149R + S214Q + V258P + A276S + H358D + | 52.4 |
| A364G + S388K + S389V | |
| V45L + A117S + L149R + S214Q + V258P + A276S + H358D + | 52.4 |
| A364G + S388K + V393H | |
| V45L + A117S + L149R + S214Q + V258P + A276Q + H358D + | 52.4 |
| A364G + S388K | |
| V45L + A117S + N127D + L149R + S214Q + V258P + A276S + | 52.4 |
| H358D + A364G + S388K | |
| V45L + L149R + W250Y + V258P + A276S + H358T + | 52.4 |
| A364G + S388K | |
| D9E + V45L + L149R + V258P + A276S + H358T + A364G + | 52.4 |
| S388K | |
| V45L + L149R + H219V + V258P + A276S + H358T + | 52.4 |
| A364G + S388K | |
| V45L + A117S + L149R + S214Q + V258P + A276S + H358D + | 52.4 |
| A364G + S388K + A472L | |
| A25W + V45L + L149R + V258P + A276S + A364G | 52.4 |
| V45L + A117S + L149R + G187K + S214Q + V258P + A276S + | 52.4 |
| A364G + S388K | |
| V45L + A117S + L149R + S214Q + V258P + A276S + A364G + | 52.4 |
| S388K + L478N | |
| V45L + A117S + L149R + S214Q + V258P + A276S + A364G + | 52.4 |
| S388K + V393R | |
| V45L + A117S + L149R + S214E + V258P + A276S + A364G + | 52.4 |
| S388K | |
| V45L + A117S + L149R + S214Q + V258P + A276S + A364G + | 52.4 |
| T366V + S388K | |
| V45L + A117S + L149R + S214Q + V258P + A276S + H358T + | 52.4 |
| A364G + S388K | |
| D11E + A25W + V45L + L149R + S214E + V258P + A276S + | 52.4 |
| A364G | |
| V45L + T70R + A117S + L149R + C161V + S214Q + V258P + | 52.4 |
| A276S + L281V + H358D + A364G + S388K + G394A + | |
| D470T + R483V | |
| V45L + A117S + L149R + T211H + S214Q + V258P + A276S + | 52.4 |
| H358D + A364G + S388K | |
| V45L + L149R + V258P + A276S + Q314A + H358T + A364G + | 52.4 |
| S388K | |
| D11E + V45L + A117S + L149R + S214Q + V258P + A276S + | 52.3 |
| A364G + S388K | |
| V45L + A117S + L149R + S214Q + V258P + A276S + A364G + | 52.3 |
| S388K + G394Q | |
| V45L + A117S + L149R + S214Q + V258P + A276S + A364G + | 52.3 |
| S388K + N395D | |
| V45L + A117S + L149R + S214Q + V258P + A276S + A364G + | 52.3 |
| S388K + V393H | |
| V45L + A117S + L149R + S214Q + V258P + A276S + A364G + | 52.3 |
| S388K + S501E | |
| V45L + A117S + L149R + S214Q + V258P + A276S + A364G + | 52.3 |
| S388K + S528H | |
| V45L + T70R + A117S + L149R + S214Q + V258P + A276S + | 52.3 |
| H358D + A364G + S388K + D470T + L530R | |
| V45L + A117S + L149R + S214Q + V258P + A276S + A364G + | 52.3 |
| S388L | |
| V45L + T70R + A117S + L149R + S214Q + V258P + A276S + | 52.3 |
| L281V + H358D + A364G + S388K + G394A + C396L + | |
| D470T + R483V | |
| V45L + L149R + T180Q + S214Q + V258P + A276S + A364G + | 52.3 |
| S388K | |
| V45L + D113T + L149R + S214Q + V258P + A276S + A364G + | 52.3 |
| S388K | |
| V45L + A117S + L149R + S214Q + V258P + A276S + H358D + | 52.3 |
| A364G + S388K + T480L | |
| V45L + A93S + L149R + A252S + H334D | 52.3 |
| V45L + A93S + L149R + V234P + A252S | 52.3 |
| V45L + A117S + L149R + S214Q + V258P + A276S + H358D + | 52.3 |
| A364G + S388H | |
| A25W + V45L + Q61R + L149R + S214E + V258P + A276S + | 52.3 |
| A364G | |
| V45L + A117S + L149R + S214Q + T215K + V258P + A276S + | 52.3 |
| H358D + A364G + S388K + D470T | |
| V45L + A117S + L149R + S214Q + V258P + A276S + A364G + | 52.3 |
| S388K + S389H | |
| V45L + A117S + L149R + S214Q + V258P + A276S + A364G + | 52.3 |
| T366I + S388K | |
| V45L + A117S + L149R + S214Q + V258P + A276S + A364G + | 52.3 |
| S388K + N474G | |
| A25W + V45L + L149R + S214E + V258P + A276S + L281V + | 52.3 |
| A364G | |
| D9E + A25W + V45L + L149R + S214E + V258P + A276S + | 52.3 |
| A364G | |
| V45L + A117S + L149R + S214Q + T215K + V258P + A276S + | 52.2 |
| H358D + A364G + S388K + D470T + L530R | |
| V45L + A117S + L149R + S214Y + V258P + A276S + H358D + | 52.2 |
| A364G + S388K | |
| V45L + A117S + L149R + S214Q + V258P + A276S + H358D + | 52.2 |
| A364G + S388K + V393R | |
| V45L + Q61R + L149R + V258P + A276S + H358T + A364G + | 52.2 |
| S388K | |
| V45L + L149R + S214Q + V258P + A276S + A364G + S388K | 52.2 |
| V45L + D113T + L149R + V258P + A276S + H358T + A364G + | 52.2 |
| S388K | |
| V45L + A117S + L149R + S214Q + V258P + A276S + A286S + | 52.2 |
| A364G + S388K | |
| V45L + A117S + L149R + S214Q + V258P + A276S + A364G + | 52.2 |
| S388R | |
| V45L + A117S + L149R + S214Q + V258P + A276S + A364G + | 52.2 |
| S388K + A499Y | |
| V45L + A117S + L149R + S214Q + V258P + A276S + A364G + | 52.2 |
| S388K + V500V | |
| V45L + Q61R + L149R + S214Q + V258P + A276S + A364G + | 52.2 |
| S388K | |
| V45L + L149R + S214E + V258P + A276S + H358T + A364G | 52.2 |
| V45L + A117S + L149R + S214Q + V258P + A276Q + A364G + | 52.2 |
| S388K | |
| V45L + L149R + V258P + A276S + L281V + A364G | 52.2 |
| V45L + A117S + L149R + S214Q + W250Y + V258P + A276S + | 52.1 |
| A364G + S388K | |
| V45L + A117S + L149R + S214Q + V258P + A276S + A364G + | 52.1 |
| S388K + T480N | |
| V45L + A117S + L149R + G187S + S214Q + V258P + A276S + | 52.1 |
| A364G + S388K | |
| A25W + V45L + A117S + L149R + S214Q + V258P + A276S + | 52.1 |
| A364G + S388K | |
| V45L + A117S + L149R + S214Q + V258P + A276S + A364G + | 52.1 |
| S388K | |
| V45L + A117S + L149R + S214Q + V258P + A276S + A364G + | 52.1 |
| S388K + N476N | |
| A25W + V45L + L149R + S214E + V258P + A276Q + A364G | 52.1 |
| V45L + A117S + L149R + S214Q + V258P + A276S + A364G + | 52.1 |
| S388K + N476D | |
| V45L + L149R + S214Q + V258P + A276S + L281V + A364G | 52.1 |
| V45L + A93S + L149R + A252S + H334D + A340G | 52.1 |
| V45L + A93S + L149R + A252S + H334D | 52.1 |
| V45L + A93S + L149R + A252S + H334D | 52.1 |
| V45L + L149R + A252S + H334D | 52.1 |
| V45L + N127D + L149R + V258P + A276S + A364G + S388K | 52.1 |
| V45L + L149R + V258P + A276S + H358T + A364G + S388K | 52.1 |
| V45L + L149R + V258P + A276S + A364G + S388K | 52.1 |
| D9E + V45L + A117S + L149R + S214Q + V258P + A276S + | 52.1 |
| A364G + S388K | |
| V45L + A117S + L149R + S214Q + V258P + A276S + A364G + | 52.1 |
| S388K + S481S | |
| V45L + L149R + N168G + V258P + A276S + A364G | 52.1 |
| V45L + L149R + T180Q + V258P + A276S + H358T + A364G + | 52.0 |
| S388K | |
| V45L + L149R + G187L + V258P + A276S + A364G + S388K | 52.0 |
| V45L + L149R + W250Y + V258P + A276S + A364G + S388K | 52.0 |
| V45L + L149R + V258P + A276S + A286E + A364G + S388K | 52.0 |
| V45L + L149R + V258P + A276S + S282N + A364G + S388K | 52.0 |
| A25W + V45L + L149R + V258P + A276S + A364G + S388K | 52.0 |
| V45L + L149R + S214Q + V258P + A276S + H358T + A364G | 52.0 |
| V45L + L149R + S173Y + V258P + A276S + A364G | 52.0 |
| V45L + A117S + L149R + S214Q + H219V + V258P + A276S + | 52.0 |
| A364G + S388K | |
| V45L + A93S + L149R + A252S + H334D | 52.0 |
| V45L + A93S + L149R + A252S + H334D + A340G | 52.0 |
| V45L + L149R + S190E + D446T + R459V | 52.0 |
| A25W + V45L + L149R + V258P + A276S + H358T | 52.0 |
| A25W + V45L + S92D + L149R + S214E + V258P + A276S + | 52.0 |
| A364G | |
| A25W + V45L + L149R + S214E + V258P + A276S + V350L + | 52.0 |
| A364G | |
| A25W + V45L + L149R + S214Q + V258P + A276S + A364G | 52.0 |
| V45L + L149R + N168G + V258P + A276S + A364G + S388K | 52.0 |
| V45L + A117S + L149R + V258P + A276S + A364G + S388K | 52.0 |
| A25W + V45L + L149R + V258P + A276S + H358T + A364G | 52.0 |
| V45L + L149R + V258P + N260H + A276S + A364G + S388K | 52.0 |
| V45L + L149R + T215I + V258P + A276S + A364G + S388K | 52.0 |
| A25W + V45L + L149R + V258P + A276Q + A364G | 52.0 |
| V45L + T70R + A117S + L149R + C161L + S214Q + V258P + | 51.9 |
| A276S + L281V + H358D + A364G + S388K + G394A + | |
| D470T +R483V | |
| A25W + V45L + L149R + S214E + V258P + A276S | 51.9 |
| V45L + A117S + L149R + S214Q + V258P + A276S + A286S + | 51.9 |
| H358D + A364G + S388K | |
| V45L + L149R + T211H + S214Q + V258P + A276S + A364G + | 51.9 |
| S388K | |
| V45L + Q61K + L149R + V258P + A276S + H358T + A364G | 51.9 |
| V45L + L149R + V258P + A276S + Q314A + H358T + A364G | 51.9 |
| V45L + L149R + T180Q + V258P + A276S + A364G + S388K | 51.9 |
| V45L + L149R + V258P + A276S + Q314A + A364G + S388K | 51.9 |
| A25W + V45L + L149R + V258P + A276S + A286S + A364G | 51.9 |
| V45L + L149R + S190E + D446T | 51.9 |
| V45L + L149R + V258P + A276S + A364G | 51.9 |
| V45L + L149R + A170S + V258P + A276S + A364G | 51.9 |
| V45L + L149R + V258P + A276Q + A364G | 51.9 |
| V45L + T70R + A117S + L149R + S214Q + V258P + A276S + | 51.9 |
| L281V + H358D + A364G + S388K + G394A + D470T + | |
| R483V +C522A | |
| V45L + L149R + H219I + V258P + A276S + H358T + A364G | 51.8 |
| V45L + L149R + V258P + A276S + V350L + A364G + S388K | 51.8 |
| D11E + V45L + L149R + V258P + A276S + A364G + S388K | 51.8 |
| V45L + D113T + L149R + V258P + A276S + A364G + S388K | 51.8 |
| V45L + L149R + G187K + V258P + A276S + A364G + S388K | 51.8 |
| M28P + V45L + L149R + V258P + A276S + A364G + S388K | 51.8 |
| V45L + Q61R + A117S + L149R + S214Q + V258P + A276S + | 51.8 |
| H358D + A364G + S388K + D470T + R483V | |
| A25W + V45L + L149R + S214Q + V258P + A276S | 51.8 |
| V45L + A117S + L149R + S214Q + T215I + V258P + A276S + | 51.8 |
| A364G + S388K | |
| A25W + V45L + L149R + V258P + A276S + S388K | 51.8 |
| A25W + V45L + L149R + S214E + H219I + V258P + A276S + | 51.8 |
| A364G | |
| V45L + A93S + L149R + V234P + A252S + H334D | 51.8 |
| V45L + L149R + T211H + V258P + A276S + H358T + A364G + | 51.8 |
| S388K | |
| A25W + V45L + A117S + L149R + S214E + V258P + A276S + | 51.8 |
| A364G | |
| V45L + L149R + S173Y + V258P + A276S + A364G + S388K | 51.8 |
| A25W + V45L + L149R + V258P + A276S + Q314A + A364G | 51.8 |
| V45L + L149R + G187L + V258P + A276S + H358T + A364G | 51.8 |
| V45L + L149R + T215V + V258P + A276S + H358T + A364G | 51.8 |
| V45L + T70R + L149R + V258P + A276S + A364G + S388K | 51.8 |
| V45L + N127D + L149R + V258P + A276S + A364G | 51.8 |
| V45L + A117S + L149R + S214Q + V258P + A276S + A364G + | 51.7 |
| S388K + Q496R | |
| V45L + L149R + W250Y + V258P + A276S + A364G | 51.7 |
| V45L + L149R + V258P + A276S + V350L + H358T + A364G | 51.7 |
| D9E + V45L + L149R + V258P + A276S + H358T + A364G | 51.7 |
| V45L + L149R + T211H + V258P + A276S + H358T + A364G | 51.7 |
| A25W + V45L + L149R + W250Y + A276Q + A364G | 51.7 |
| V45L + L149R + V258P + A276S + L281V + A364G + S388K | 51.7 |
| V45L + L149R + V258P + A276S + S282N + H358T + A364G | 51.7 |
| V45L + L149R + T215I + V258P + A276S + H358T + A364G | 51.7 |
| A25W + V45L + L149R + S173Y + V258P + A276S + A364G | 51.7 |
| V45L + Q61K + L149R + V258P + A276S + A364G + S388K | 51.7 |
| V45L + L149R + T180Q + V258P + A276S + A364G | 51.7 |
| V45L + A117S + L149R + A170S + S214Q + V258P + A276S + | 51.7 |
| A364G + S388K | |
| V45L + Q61R + A117S + L149R + S214Q + V258P + A276S + | 51.7 |
| A364G + S388K | |
| A25W + V45L + L149R + S214E + T215V + V258P + A276S + | 51.7 |
| A364G | |
| V45L + A117S + L149R + S214Q + V258P + A276S + A364G + | 51.7 |
| S388K + V497P | |
| V45L + L149R + V258P + A276S + Q314A + A364G | 51.7 |
| V45L + A117S + L149R + V258P + A276S + A364G | 51.7 |
| V45L + A117S + L149R + V258P + A276S + H358T + A364G + | 51.7 |
| S388K | |
| V45L + L149R + N168G + V258P + A276S + H358T + A364G + | 51.7 |
| S388K | |
| A25W + V45L + Q61K + L149R + V258P + A276S + A364G | 51.6 |
| V45L + L149R + S214E + V258P + A276S + L281V + A364G | 51.6 |
| V45N + L149R + V258P + A276S + A364G + S388K | 51.6 |
| V45L + L149R + V258P + A276S + A286E + H358T + A364G | 51.6 |
| D9E + V45L + L149R + V258P + A276S + A364G + S388K | 51.6 |
| V45L + S92D + L149R + V258P + A276S + A364G + S388K | 51.6 |
| V45L + A117S + L149R + T180Q + S214Q + V258P + A276S + | 51.6 |
| A364G + S388K | |
| V45L + A117S + L149R + S214Q + V258P + A276S + A364G + | 51.6 |
| S388H | |
| V45L + A117S + L149R + S214Q + T215V + V258P + A276S + | 51.6 |
| A364G + S388K | |
| V45L + A117S + L149R + S214Q + V258P + A276S + V350L + | 51.6 |
| A364G + S388K | |
| A25W + V45L + L149R + S214E + T215I + V258P + A276S + | 51.6 |
| A364G | |
| V45L + N127D + L149R + V258P + A276S + H358T + A364G + | 51.6 |
| S388K | |
| A25W + V45L + L149R + V258P + A276S + A286E + A364G | 51.6 |
| V45L + L149R + N168G + V258P + A276S + L281V + A364G | 51.6 |
| V45L + Q61R + L149R + V258P + A276S + H358T + A364G | 51.6 |
| V45L + L149R + G187S + V258P + A276S + A364G + S388K | 51.6 |
| V45L + L149R + V258P + A276S + L281V + Q314A + A364G | 51.6 |
| V45L + L149R + V258P + A276S + L281V + H358T + A364G | 51.6 |
| V45L + L149R + A170S + V258P + A276S + H358T + A364G | 51.6 |
| V45L + L149R + A170S + V258P + A276S + A364G + S388K | 51.6 |
| V45L + L149R + T215V + V258P + A276S + A364G + S388K | 51.6 |
| V45L + L149R + V258P + A276Q + L281V + A364G | 51.6 |
| V45L + T70R + A117S + L149R + S214Q + C243S + V258P + | 51.5 |
| A276S + L281V + H358D + A364G + S388K + G394A + | |
| D470T +R483V | |
| V45L + T70R + A117S + L149R + S214Q + V258P + A276S + | 51.5 |
| L281V + H358D + A364G + S388K + G394A + D470T + | |
| R483V +C522V | |
| A25W + V45L + L149R + V258P + A276S + Q314A | 51.5 |
| V45L + L149R + V258P + A276Q + H358T + A364G + S388K | 51.5 |
| A25W + V45N + L149R + S214E + V258P + A276S + A364G | 51.5 |
| V45L + L149R + V258P + A276S + A286E + A364G | 51.5 |
| V45L + L149R + V258P + A276S + A286S + H358T + A364G | 51.5 |
| A25W + V45L + L149R + G187S + V258P + A276S + A364G | 51.5 |
| V45L + L149R + T180Q + V258P + A276S + L281V + A364G | 51.5 |
| A25W + V45L + L149R + A276Q + L281V + A364G | 51.5 |
| V45L + S92D + L149R + V258P + A276S + L281V + A364G | 51.5 |
| V45L + Q61R + L149R + V258P + A276S + L281V + A364G | 51.5 |
| V45L + L149R + A170S + V258P + A276S + L281V + A364G | 51.5 |
| D11E + A25W + V45L + L149R + V258P + A276S + A364G | 51.5 |
| A25W + V45L + Q61R + L149R + V258P + A276S + A364G | 51.5 |
| A25W + V45L + L149R + T215V + V258P + A276S + A364G | 51.5 |
| D9E + V45L + L149R + V258P + A276S + L281V + A364G | 51.5 |
| A25W + V45L + L149R + V258P + A276S + V350L + A364G | 51.5 |
| V45L + Q61K + A117S + L149R + S214Q + V258P + | 51.5 |
| A276S +A364G + S388K | |
| V45L + A117S + L149R + S214Q + V258P + A276S + | 51.5 |
| A364G +S388K + Y391V | |
| V45L + A117S + L149R + N168G + S214Q + V258P + | 51.5 |
| A276S +A364G + S388K | |
| V45L + D113T + A117S + L149R + S214Q + V258P + | 51.5 |
| A276S +A364G + S388K | |
| A25W + V45L + L149R + V258P + A276S + A286E | 51.5 |
| A25W + V45L + L149R + V258P + A276S + L281V | 51.5 |
| A25W + V45L + D113T + L149R + V258P + A276S | 51.5 |
| V45L + L149R + H219V + V258P + A276S + L281V + A364G | 51.5 |
| V45L + L149R + T180Q + V258P + A276S + H358T + A364G | 51.5 |
| V45L + L149R + T215V + V258P + A276S + L281V + A364G | 51.5 |
| A25W + V45L + L149R + V258P + A276S + S282N + A364G | 51.5 |
| A25W + V45L + L149R + T215I + V258P + A276S + A364G | 51.5 |
| A25W + V45L + L149R + H219I + V258P + A276S + A364G | 51.5 |
| V45L + A117S + L149R + V258P + A276S + L281V + A364G | 51.5 |
| D9E + A25W + V45L + L149R + V258P + A276S + A364G | 51.5 |
| V45L + L149R + S214Y + V258P + A276S + A364G + S388K | 51.5 |
| A25W + V45L + L149R + T180Q + V258P + A276S + A364G | 51.5 |
| A25W + V45L + L149R + V258P + A276S + L281V + A364G | 51.5 |
| V45L + N127D + L149R + V258P + A276S + L281V + A364G | 51.5 |
| V45L + L149R + S173Y + V258P + A276S + H358T + A364G | 51.5 |
| V45L + Q61R + L149R + V258P + A276S + A364G + S388K | 51.5 |
| M28P + V45L + A117S + L149R + S214Q + V258P + A276S + | 51.4 |
| A364G + S388K | |
| V45L + A117S + L149R + S214Q + V258P + A276S + A364G + | 51.4 |
| S388A | |
| V45N + A117S + L149R + S214Q + V258P + A276S + A364G + | 51.4 |
| S388K | |
| V45L + A117S + L149R + S214Q + V258P + A276S + A364G + | 51.4 |
| S388K + T477K | |
| A25W + V45L + L149R + T180Q + V258P + A276S | 51.4 |
| A25W + V45L + L149R + V258P + A276S + A286S | 51.4 |
| A25W + V45L + L149R + G187S + S214E + V258P + A276S + | 51.4 |
| A364G | |
| V45L + L149R + V258P + A276S + L281V + A286E + A364G | 51.4 |
| A25W + V45L + L149R + W250Y + V258P + A276S + A364G | 51.4 |
| A25W + V45L + L149R + V258P + N260H + A276S + A364G | 51.4 |
| V45L + L149R + V258P + A276S + L281V + V350L + A364G | 51.4 |
| A25W + V45L + L149R + A170S + V258P + A276S + A364G | 51.4 |
| V45L + L149R + V258P + A276S + L281V + A286S + A364G | 51.4 |
| V45L + L149R + V258P + A276S + L281V + S282N + A364G | 51.4 |
| D11E + V45L + L149R + V258P + A276S + L281V + A364G | 51.4 |
| V45L + Q61K + L149R + V258P + A276S + L281V + A364G | 51.4 |
| V45L + L149R + T215I + V258P + A276S + L281V + A364G | 51.4 |
| A25W + V45L + S92D + L149R + V258P + A276S + A364G | 51.4 |
| V45L + L149R + S214Y + V258P + A276S + L281V + A364G | 51.4 |
| V45L + L149R + T211H + V258P + A276S + L281V + A364G | 51.4 |
| A25W + V45L + L149R + G187L + V258P + A276S + A364G | 51.4 |
| V45L + S92D + L149R + V258P + A276S + H358T + A364G | 51.4 |
| V45L + L149R + G187L + A276Q + L281V + A364G | 51.4 |
| V45L + L149R + A276Q + L281V + V350L + A364G | 51.4 |
| V45L + L149R + A276Q + L281V + A364G + S388K | 51.4 |
| V45L + A117S + L149R + S214Y + V258P + A276S + A364G + | 51.4 |
| S388K | |
| A25W + V45L + S92D + L149R + V258P + A276S | 51.4 |
| A25W + V45L + L149R + G187L + V258P + A276S | 51.4 |
| A25W + V45L + L149R + W250Y + V258P + A276S | 51.4 |
| V45L + D113T + L149R + V258P + A276S + A364G | 51.3 |
| V45L + L149R + G187K + V258P + A276S + H358T + A364G | 51.3 |
| V45L + L149R + N163H + V258P + A276S + L281V + A364G | 51.3 |
| A25W + V45L + T70R + L149R + V258P + A276S + A364G | 51.3 |
| V45L + L149R + S173Y + V258P + A276S + L281V + A364G | 51.3 |
| V45L + L149R + G187L + V258P + A276S + L281V + A364G | 51.3 |
| V45L + L149R + G187K + V258P + A276S + L281V + A364G | 51.3 |
| V45L + L149R + W250Y + V258P + A276S + H358T + A364G | 51.3 |
| V45L + L149R + T211H + V258P + A276S + A364G + S388K | 51.3 |
| V45L + L149R + N163H + V258P + A276S + A364G + S388K | 51.3 |
| V45L + T70R + L149R + A276Q + L281V + A364G | 51.3 |
| V45L + S92D + L149R + A276Q + L281V + A364G | 51.3 |
| V45L + D113T + L149R + A276Q + L281V + A364G | 51.3 |
| V45L + L149R + A276Q + L281V + Q314A + A364G | 51.3 |
| V45L + A117S + L149R + S214Q + V258P + A276S + A364G + | 51.3 |
| S388K + S389V | |
| A25W + V45L + Q61K + L149R + V258P + A276S | 51.3 |
| D9E + A25W + V45L + L149R + V258P + A276S | 51.3 |
| V45L + T70R + L149R + V258P + A276S + A364G | 51.3 |
| V45L + L149R + V258P + A276S + A286S + A364G + S388K | 51.3 |
| A25W + V45L + L149R + S214Y + V258P + A276S + A364G | 51.3 |
| A25W + V45L + D113T + L149R + V258P + A276S + A364G | 51.3 |
| V45L + T70R + L149R + V258P + A276S + L281V + A364G | 51.3 |
| A25W + V45L + N127D + L149R + V258P + A276S + A364G | 51.3 |
| V45L + T70R + L149R + V258P + A276S + H358T + A364G | 51.3 |
| V45L + L149R + N163H + V258P + A276S + A364G | 51.3 |
| V45L + L149R + T180Q + A276Q + L281V + A364G | 51.2 |
| V45L + L149R + A276Q + L281V + A286S + A364G | 51.2 |
| A25W + V45L + L149R + A170S + V258P + A276S | 51.2 |
| A25W + V45L + T70R + L149R + V258P + A276S | 51.2 |
| A25W + V45L + L149R + S214E + V258P + A276S + A286S + | 51.2 |
| A364G | |
| V45L + L149R + V258P + A276S + A286S + H358T + A364G + | 51.2 |
| S388K | |
| V45L + L149R + H219I + V258P + A276S + L281V + A364G | 51.2 |
| A25W + V45L + A117S + L149R + V258P + A276S + A364G | 51.2 |
| A25W + V45L + L149R + N168G + V258P + A276S + A364G | 51.2 |
| V45L + L149R + N168G + A276Q + L281V + A364G | 51.2 |
| V45L + L149R + S173Y + A276Q + L281V + A364G | 51.2 |
| V45L + L149R + W250Y + A276Q + L281V + A364G | 51.2 |
| V45L + A117S + L149R + S173Y + S214Q + V258P + A276S + | 51.2 |
| A364G + S388K | |
| A25W + V45L + L149R + A276Q + A286E + A364G | 51.1 |
| A25W + V45L + L149R + A276Q + A364G + S388K | 51.1 |
| V45L + N127D + L149R + V258P + A276S + H358T + A364G | 51.1 |
| A25W + V45L + L149R + V258P + A276S | 51.1 |
| V45L + N127D + L149R + A276Q + L281V + A364G | 51.1 |
| V45L + L149R + A276Q + L281V + A286E + A364G | 51.1 |
| V45L + A117S + L149R + S214Q + V258P + A276S + A364G + | 51.1 |
| S388K + V500T | |
| A25W + V45L + A117S + L149R + V258P + A276S | 51.1 |
| A25W + V45L + L149R + H219V + V258P + A276S + A364G | 51.1 |
| V45L + L149R + S173Y + V258P + A276S + H358T + A364G + | 51.1 |
| S388K | |
| D11E + V45L + L149R + V258P + A276S + H358T + A364G | 51.1 |
| V45L + A79C + L149R + V258P + A276S + N355G + A364G + | 51.1 |
| S388K | |
| A24K + V45L + L149R + V258P + A276S + A364G | 51.1 |
| V45L + L149R + V258P + A276S + S282N + A364G | 51.1 |
| V45L + A117S + L149R + A276Q + L281V + A364G | 51.0 |
| V45L + L149R + N260H + A276Q + L281V + A364G | 51.0 |
| V45L + A117S + L149R + T211H + S214Q + V258P + A276S + | 51.0 |
| A364G + S388K | |
| A25W + V45L + L149R + N168G + V258P + A276S | 51.0 |
| A25W + V45L + L149R + G187S + V258P + A276S | 51.0 |
| A25W + V45L + L149R + V258P + A276S + V350L | 51.0 |
| A25W + V45L + Q61R + L149R + V258P + A276S | 51.0 |
| A25W + V45L + L149R + G187L + S214E + V258P + A276S + | 51.0 |
| A364G | |
| V45L + L149R + G187S + V258P + A276S + L281V + A364G | 51.0 |
| A25W + V45L + L149R + N163H + V258P + A276S + A364G | 51.0 |
| A25W + V45L + L149R + G187K + V258P + A276S + A364G | 51.0 |
| V45N + L149R + V258P + A276S + L281V + A364G | 51.0 |
| V45L + L149R + A276Q + L281V + A364G | 51.0 |
| V45L + L149R + V258P + N260H + A276S + H358T + A364G | 51.0 |
| V45L + L149R + A276Q + S282N + A364G | 51.0 |
| V45L + S92D + A117S + L149R + S214Q + V258P + A276S + | 51.0 |
| A364G + S388K | |
| A25W + V45L + L149R + V258P + A276S + S282N | 51.0 |
| A25W + V45L + L149R + S173Y + V258P + A276S | 51.0 |
| A25W + V45L + L149R + A276Q + A286S + A364G | 50.9 |
| V45L + D113T + L149R + V258P + A276S + L281V + A364G | 50.9 |
| V45L + L149R + S214Q + V258P + A276S + L281V + A364G + | 50.9 |
| S388K | |
| V45L + D113T + L149R + V258P + A276S + H358T + A364G | 50.9 |
| A25W + V45L + L149R + T215V + V258P + A276S | 50.9 |
| V45L + L149R + N168G + V258P + A276S + H358T + A364G | 50.9 |
| V45L + A117S + L149R + S214Q + V258P + A276S + A364G + | 50.9 |
| S388K + A472L | |
| V45L + A117S + L149R + S214Q + V258P + A276S + A364G + | 50.9 |
| S388K + S473K | |
| A25W + V45L + L149R + T215I + V258P + A276S | 50.9 |
| A25W + V45L + L149R + T211H + V258P + A276S | 50.9 |
| A25W + V45L + L149R + V258P + A276Q | 50.9 |
| V45L + L149R + S214Q + T215V + V258P + A276S + A364G + | 50.9 |
| S388K | |
| V45L + L149R + N163H + V258P + A276S + H358T + A364G + | 50.9 |
| S388K | |
| V45L + A117S + L149R + S214Q + V258P + A276S + A364G + | 50.8 |
| S388K + A499L | |
| V45L + L149R + V258P + A276S + V350L + A364G | 50.8 |
| V45L + A79C + A117S + L149R + V258P + A276S + A364G + | 50.8 |
| S388K | |
| A25W + V45L + D113T + L149R + A276Q + A364G | 50.8 |
| A25W + V45N + L149R + V258P + A276S + A364G | 50.8 |
| V45L + T70R + L149R + A276Q + A364G | 50.8 |
| A25W + V45L + L149R + H219I + V258P + A276S | 50.8 |
| V45L + A117S + L149R + S214Q + V258P + A276S + A364G + | 50.8 |
| S388K + V498I | |
| M28P + V45L + L149R + V258P + A276S + A364G | 50.8 |
| A25W + V45L + T70R + L149R + A276Q + A364G | 50.8 |
| A25W + V45L + L149R + A276Q + Q314A + A364G | 50.8 |
| V45L + L149R + A170S + V258P + A276S + H358T + A364G + | 50.7 |
| S388K | |
| A25W + V45L + L149R + S214Y + V258P + A276S | 50.7 |
| V45L + L149R + S214Y + V258P + A276S + H358T + A364G | 50.7 |
| V45L + L149R + A276Q + L281V + H358T + A364G | 50.7 |
| A25W + V45L + L149R + H219V + V258P + A276S | 50.7 |
| V45L + A117S + L149R + N163H + S214Q + V258P + A276S + | 50.7 |
| A364G + S388K | |
| D11E + A25W + V45L + L149R + V258P + A276S | 50.7 |
| V45L + A117S + L149R + S214Q + V258P + A276S + A364G + | 50.7 |
| S388K + N527H | |
| A25W + V45L + A117S + L149R + A276Q + A364G | 50.7 |
| V45L + L149R + V258P + L281V + A364G | 50.7 |
| A25W + V45L + L149R + G187L + A276Q + A364G | 50.6 |
| V45N + L149R + V258P + A276S + H358T + A364G | 50.6 |
| V45L + A117S + L149R + S214Q + V258P + A276S + N355G + | 50.6 |
| H358D + A364G + S388K + D470T + R483V | |
| V45L + A117S + L149R + S214Q + V258P + A276S + H358D + | 50.6 |
| A364G + S388H + D470T + R483V | |
| A25W + V45L + L149R + A276Q + A364G | 50.6 |
| V45L + A117S + L149R + S214Q + V258P + A276S + H358D + | 50.6 |
| A364G + S388K + D470T + R483V + V497E | |
| A25W + V45L + L149R + N163H + A276Q + A364G | 50.6 |
| A25W + V45L + L149R + S173Y + A276Q + A364G | 50.6 |
| A25W + V45L + L149R + A276Q + S282N + A364G | 50.6 |
| A25W + V45L + L149R + A276Q + H358T + A364G | 50.6 |
| A25W + V45L + L149R + T180Q + A276Q + A364G | 50.6 |
| V45L + A117S + L149R + V258P + A276S + H358T + A364G | 50.5 |
| V45L + L149R + A276Q + Q314A + A364G | 50.5 |
| V45L + L149R + G187K + V258P + A364G | 50.5 |
| V45L + L149R + A276Q + A286E + A364G | 50.5 |
| A25W + V45L + N127D + L149R + A276Q + A364G | 50.5 |
| A25W + V45L + L149R + A170S + A276Q + A364G | 50.5 |
| A25W + V45L + N127D + L149R + V258P + A276S | 50.5 |
| V45L + A117S + L149R + S214Q + V258P + A276S + A364G + | 50.4 |
| S388K + T477V | |
| V45L + A117S + L149R + S214Q + V258P + A276S + A364G + | 50.4 |
| S388K + G503L | |
| A25W + M28P + V45L + L149R + V258P + A276S + A364G | 50.4 |
| V45L + L149R + A276Q + H358T + A364G | 50.4 |
| A25W + V45L + L149R + N168G + A276Q + A364G | 50.4 |
| A25W + V45L + L149R + N260H + A276Q + A364G | 50.4 |
| V45L + A117S + L149R + G187L + S214Q + V258P + A276S + | 50.4 |
| A364G + S388K | |
| V45L + A117S + L149R + N163H + S214Q + V258P + A276S + | 50.3 |
| H358D + A364G + S388K + D470T + L530R | |
| V45L + A117S + L149R + S214Q + T215K + V258P + A276S + | 50.3 |
| A364G + S388K | |
| A25W + V45L + L149R + G187K + V258P + A276S | 50.3 |
| A25W + M28P + V45L + L149R + V258P + A276S | 50.3 |
| A25W + V45N + L149R + V258P + A276S | 50.3 |
| V45L + A117S + L149R + S214Q + V258P + A276S + N355G + | 50.2 |
| H358D + A364G + S388K + D470T | |
| V45L + N127D + L149R + A276Q + A364G | 50.2 |
| V45L + L149R + W250Y + A276Q + A364G | 50.2 |
| V45L + L149R + W250Y + V258P + A364G | 50.2 |
| V45L + L149R + N163H + A276Q + L281V + A364G | 50.1 |
| V45L + A117S + L149R + S214Q + V258P + A276S + A364G + | 50.1 |
| Q385C + S388K | |
| A25W + V45L + L149R + S214E + T215K + V258P + A276S + | 50.1 |
| A364G | |
| M28P + V45L + L149R + V258P + A276S + L281V + A364G | 50.0 |
| V45L + L149R + A276Q + A364G + S388K | 50.0 |
| A25W + V45L + L149R + V258P + A364G | 50.0 |
| M28P + V45L + L149R + V258P + A276S + H358T + A364G | 50.0 |
| V45L + A117S + L149R + S214Q + V258P + A276S + N355G + | 50.0 |
| H358D + A364G + S388K | |
| V45L + T70R + A117S + L149R + S214Q + V258P + A276S + | 50.0 |
| L281V + H358D + A364G + S388K + G394A + D470T + | |
| R483V + C540V | |
| V45L + L149R + N168G + A276Q + A364G | 50.0 |
| V45L + A117S + L149R + A276Q + A364G | 49.9 |
| V45L + L149R + N163H + V258P + A276S + H358T + A364G | 49.9 |
| V45L + L149R + T215K + V258P + A276S + H358T + A364G | 49.9 |
| A24K + V45L + L149R + A276Q + A364G | 49.9 |
| V45L + L149R + A276Q + A286S + A364G | 49.9 |
| V45L + L149R + V258P + A364G | 49.9 |
| V45L + L149R + H219I + V258P + A276S + A364G + S388K | 49.8 |
| V45L + L149R + A170S + A276Q + A364G | 49.8 |
| V45L + L149R + T180Q + A276Q + A364G | 49.8 |
| V45L + L149R + A276Q + V350L + A364G | 49.8 |
| V45L + L149R + V258P + S282N + A364G | 49.8 |
| V45L + L149R + A276Q + A364G | 49.8 |
| V45L + L149R + A276S + A364G | 49.8 |
| V45L + L149R + T215K + V258P + A276S + A364G + S388K | 49.8 |
| V45L + A117S + L149R + S214Q + V258P + A276S + N355G + | 49.8 |
| A364G + S388K + D470T | |
| V45L + L149R + V258P + A276S | 49.8 |
| A25W + V45L + L149R + A276S + A364G | 49.8 |
| V45L + L149R + G187S + V258P + A364G | 49.8 |
| V45L + S92D + L149R + V258P + A276S + H358T + A364G + | 49.7 |
| S388K | |
| A25W + L149R + V258P + A276S + A364G | 49.7 |
| V45L + L149R + G187L + A276Q + A364G | 49.7 |
| A25W + V45L + V258P + A276S + A364G | 49.6 |
| V45L + L149R + N260H + A276Q + A364G | 49.6 |
| V45L + L149R + W250Y + V258R + A364G | 49.5 |
| V45L + L149R + N163H + A276Q + A364G | 49.4 |
| V45L + S92D + L149R + A276Q + A364G | 49.4 |
| V45L + L149R + S173Y + A276Q + A364G | 49.4 |
| V45L + S92D + L149R + V258P + A364G | 49.3 |
| V45L + L149R + W250Y + V258P + A276S + L281V + A364G | 49.3 |
| V45L + L149R + L281V + A364G | 49.3 |
| V45L + D113T + L149R + A276Q + A364G | 49.2 |
| L149R + V258P + A276S + A364G | 49.2 |
| V45L + L149R + G187S + V258R + A364G | 49.1 |
| V45L + V258P + A276S + A364G | 49.1 |
| V45L + L149R + V258R + A364G + S388K | 49.1 |
| A25W + V45L + L149R + A364G | 49.0 |
| V45L + L149R + G187K + V258R + A364G | 49.0 |
| V45L + L149R + V258P | 49.0 |
| V45L + L149R + V258R + S282N + A364G | 48.9 |
| V45L + L149R | 48.8 |
| V45L + L149R + V258R + L281V + A364G | 48.7 |
| V45L + L149R + A364G | 48.7 |
| V45L + L149R + S214Q + V258P + A276S + N355G + | 48.6 |
| A364G +S388K | |
| V45L + S92N + L149R + V258P + A364G | 48.6 |
| V45L + A117S + L149R + S214Q + V258P + A276S + | 48.6 |
| A286E +H358D + A364G + S388K | |
| A25W + V45L + L149R + S214E + V258P + A276S + | 48.5 |
| N355G +A364G | |
| V45L + L149R + G187L + V258P + A276S + A364G | 48.5 |
| V45L + L149R + V258R + A364G | 48.5 |
| V45L + L149R + V258P + A364G + S388K | 48.5 |
| A25W + V45L + L149R + A276Q + V350L + A364G | 48.5 |
| V45L + L149R + H219V + V258P + A276S + A364G + S388K | 48.4 |
| V45L + L149R + G187K + A364G | 48.4 |
| V45L + L149R + G187S + A364G | 48.4 |
| V45L + A117S + L149R + S214Q + V258P + A276S + | 48.2 |
| N355G +A364G + S388K | |
| V45L + A117S + L149R + S214Q + V258P + A276S + | 48.2 |
| H358D +A364G + Y386G + S388K + D470T + R483V | |
| V45L + L149R + W250Y | 48.1 |
| V45L + S92N + L149R + V258R + A364G | 48.0 |
| A25W + V45L + L149R + V258P + A276S + N355G + A364G | 47.9 |
| V45L + L149R + V258P + A276S + N355G + A364G + S388K | 47.9 |
| V45L + L149R + G187S | 47.8 |
| V45N + L149R | 47.8 |
| V45L + L149R + S282N | 47.7 |
| L149R + A364G | 47.7 |
| Y386G | 47.7 |
| V258P + A276Q | 47.7 |
| V258P + A276S | 47.7 |
| V45L + A117S + L149R + S214Q + V258P + A276S + | 47.6 |
| H358D +A364G + S388K + G394D + D470T + R483V | |
| A25W + L149R | 47.6 |
| V45L + A276S | 47.6 |
| V45L + S92D + L149R | 47.6 |
| A25W + A276S | 47.6 |
| Q385C | 47.6 |
| V45L + L149R + W250Y + A364G | 47.5 |
| V45L + A276Q | 47.5 |
| V45L + V258P | 47.5 |
| V45L + A117S + L149R + S214Q + V258P + A276S + | 47.4 |
| H358D +A364G + Y386G + S388K | |
| A25W + V258P | 47.2 |
| A25W + A276Q | 47.1 |
| L149R + W250Y | 47.1 |
| V45L + L149R + V258P + A276S + L281V + N355G + A364G | 47.1 |
| V45L + S388K | 47.0 |
| V45L + L149R + V258P + A276S + N355G + H358T + A364G | 47.0 |
| V45L + A364G | 46.9 |
| V45N + V258P | 46.9 |
| A276S | 46.8 |
| V258P + S388K | 46.8 |
| A25W + V45L | 46.8 |
| A276Q + S388K | 46.8 |
| A276C | 46.8 |
| A276Q | 46.7 |
| A276F | 46.7 |
| W250Y + A276Q | 46.6 |
| A79C | 46.6 |
| A276S + S388K | 46.6 |
| A276Q + L281V | 46.6 |
| V45L + L149R + V258P + A276S + N355G + A364G | 46.5 |
| A276N | 46.5 |
| W250Y + A276S | 46.5 |
| A25W + S388K | 46.5 |
| A276S + L281V | 46.4 |
| G187S + A276Q | 46.3 |
| A25W + V45L + L149R + V258P + A276S + N355G | 46.3 |
| V45L | 46.3 |
| S92D + A276S | 46.3 |
| S19T | 46.2 |
| L149R + V258R | 46.2 |
| V45N + S388K | 46.2 |
| A25W + V45L + S92D + L149R + A276Q + A364G | 46.2 |
| V45L + A117S + L149R + S214Q + V258P + A276S + | 46.1 |
| A364G +S388K + D470T + S501E | |
| H358D | 46.1 |
| A25W + A364G | 46.0 |
| V258P + L281V | 46.0 |
| L149R | 46.0 |
| V45L + L149R + H219V + V258P + A276S + H358T + A364G | 46.0 |
| A25W | 46.0 |
| V258R + A276S | 46.0 |
| V45N | 45.9 |
| G187S + A276S | 45.8 |
| G187K + A276S | 45.8 |
| I469T | 45.8 |
| V45L + S92D | 45.8 |
| V45L + L281V | 45.8 |
| V45N + W250Y | 45.8 |
| V258R + A276Q | 45.8 |
| V45L + V258R | 45.8 |
| W250Y + S388K | 45.7 |
| A25W + V45L + L149R + A276Q + N355G + A364G | 45.6 |
| V45N + G187K | 45.6 |
| A25W + V45L + L149R + S214E + V258P + A276S + | 45.6 |
| Q314A +A364G | |
| S388A | 45.6 |
| A276T | 45.6 |
| A25W + V258R | 45.6 |
| G187K + S388K | 45.6 |
| A25W + S92D | 45.6 |
| L281V + S388K | 45.6 |
| V45N + V258R | 45.6 |
| S388H | 45.6 |
| S92D | 45.6 |
| D525L | 45.5 |
| S214Q | 45.5 |
| L281V + A364G | 45.5 |
| A364G | 45.5 |
| S388L | 45.5 |
| S388R | 45.5 |
| S473G | 45.5 |
| S92D + A364G | 45.5 |
| A25W + G187S | 45.4 |
| A25W + G187K | 45.4 |
| W250Y + V258P | 45.4 |
| S92D + V258P | 45.4 |
| S389V | 45.4 |
| A20H | 45.4 |
| W495E | 45.4 |
| G187K + W250Y | 45.4 |
| Y386Y | 45.4 |
| V393H | 45.4 |
| G187L | 45.4 |
| Q61K | 45.3 |
| V45N + S92D | 45.3 |
| G187S + A364G | 45.3 |
| G187S + L281V | 45.3 |
| G187S | 45.3 |
| A286E | 45.3 |
| A276Y | 45.3 |
| V258P | 45.3 |
| Q496E | 45.3 |
| G394A | 45.3 |
| V393R | 45.3 |
| G187S + S388K | 45.3 |
| T366V | 45.3 |
| S389S | 45.2 |
| S92N | 45.2 |
| N527H | 45.2 |
| Q384A | 45.2 |
| V497D | 45.2 |
| D470T | 45.2 |
| A18V | 45.2 |
| Q496Q | 45.2 |
| Q496R | 45.2 |
| S214E | 45.2 |
| P362P | 45.2 |
| S389H | 45.2 |
| V258R | 45.2 |
| A276D | 45.2 |
| L281V | 45.2 |
| V45I | 45.2 |
| A117C | 45.2 |
| N395N | 45.2 |
| Y391Y | 45.2 |
| A24H | 45.2 |
| A472I | 45.2 |
| D470K | 45.1 |
| S473K | 45.1 |
| S473S | 45.1 |
| W495I | 45.1 |
| V497E | 45.1 |
| A24K | 45.1 |
| V45T | 45.1 |
| L471R | 45.1 |
| A472L | 45.1 |
| A20P | 45.1 |
| N395D | 45.1 |
| G394D | 45.1 |
| G394Q | 45.1 |
| G187S + V258R | 45.1 |
| S92D + G187S | 45.1 |
| V45C | 45.0 |
| N476N | 45.0 |
| G207G | 45.0 |
| A364A | 45.0 |
| D470A | 45.0 |
| N474G | 45.0 |
| D470V | 45.0 |

### Example 3: Thermostability of Xylanase Variants Using Nano-Differential Scanning Fluorimetry (DSF)

Culture supernatants grown in 96-well plate was clarified by centrifugation (5424R, Eppendorf) at 3000×g for 30 min at 4° C. Clarified supernatant thus obtained was diluted 5-times with dilution buffer in 1.5 ml Eppendorf tube (MCT-15-A, Axygen^{R}). Dilution buffer: sodium acetate buffer (50 mM) was made by adding 4.1 g of sodium Acetate (Sigma, S2889) in 1 L of Milli-Q water and adjusting the pH to 4.0 using acetic acid. Diluted samples were subjected to centrifugation at 12,000×g for 2 min to remove any precipitates formed. Clarified supernatants were filled in nano-DSF glass capillaries (PR-0002, Nanotemper Technologies) by dipping capillary in the sample and placed in a slot if the nano-DSF instrument (Prometheus NT.48, Nanotemper Technologies). Each sample was analyzed in triplicate. Hence, total 16 samples were analyzed in 48 capillaries in a single run. In each run, purified xylanase-WT and culture supernatant-WT were used as controls. Samples were scanned from 25-65° C. at a ramp speed of 1.5° C./min. Melting temperature (Tₘ) was obtained from first-derivative curve plotted from raw data using inbuilt software provided by Nanotemper Technologies.

Tm of the xylanase variant and the Tm of the wild-type xylanase (SEQ ID NO: 1) are presented in table 2 below. The results in table 2 shows that the xylanase variants have improved thermostability relative to the wild-type xylanase (SEQ ID NO: 1).

TABLE 2
Thermostability of xylanase variants
of SEQ ID NO: 1 using nano-DSF

| | Tm |
|---|---|
| Variant(s) | (° C.) |
| WT | 43.6 |
| A25W + V45L + L149R + V258P + A276S + A364G | 51.5 |
| V45L + L149R + V258P + A276S + L281V + A364G | 46.4 |
| V45L + L149R + V258P + A276S + A364G + S388K | 52.6 |
| V45L + L149R + S173Y + V258P + A276S + A364G | 51.9 |
| V45L + L149R + V258P + A276S + A364G | 52.1 |
| V45L + L149R + A170S + V258P + A276S + A364G | 51.6 |
| V45L + L149R + V258P + A276Q + A364G | 51.7 |
| V45L + N127D + L149R + V258P + A276S + A364G | 51.9 |
| V45L + L149R + W250Y + V258P + A276S + A364G | 51.7 |
| V45L + L149R + T180Q + V258P + A276S + A364G | 51.9 |
| V45L + L149R + V258P + A276S + Q314A + A364G | 51.9 |
| V45L + A117S + L149R + V258P + A276S + A364G | 52.0 |
| V45L + L149R + V258P + A276S + A286E + A364G | 51.5 |
| V45L + T70R + L149R + V258P + A276S + A364G | 52.2 |
| V45L + L149R + N163H + V258P + A276S + A364G | 51.2 |
| V45L + L149R + A276Q + L281V + A364G | 51.4 |
| V45L + L149R + V258P + A276S + V350L + A364G | 51.2 |
| M28P + V45L + L149R + V258P + A276S + A364G | 51.1 |
| V45L + L149R + V258P + L281V + A364G | 51.9 |
| A25W + V45L + L149R + A276Q + A364G | 51.4 |
| V45L + L149R + A276Q + Q314A + A364G | 49.2 |
| V45L + L149R + G187K + V258P + A364G | 51.7 |
| V45L + L149R + A276Q + H358T + A364G | 49.9 |
| V45L + L149R + W250Y + A276Q + A364G | 48.6 |
| V45L + A117S + L149R + A276Q + A364G | 49.4 |
| V45L + L149R + A276Q + A286S + A364G | 49.2 |
| V45L + L149R + A170S + A276Q + A364G | 49.3 |
| V45L + L149R + T180Q + A276Q + A364G | 50.0 |
| V45L + L149R + A276Q + V350L + A364G | 48.9 |
| V45L + L149R + G187S + V258P + A364G | 49.7 |
| V45L + L149R + G187L + A276Q + A364G | 49.5 |
| V45L + L149R + N163H + A276Q + A364G | 48.7 |
| V45L + L149R + S173Y + A276Q + A364G | 47.4 |
| V45L + L149R | 48.3 |
| V45L + L149R + G187L + V258P + A276S + A364G | 51.7 |
| V45L + L149R + V258P + A364G + S388K | 49.6 |
| V45L + L149R + V258P + A276S + N355G + A364G | 46.5 |
| V45L | 46.3 |
| L149R | 46.5 |
| A79C | 47.6 |
| V45N | 46.5 |
| V45L + D11E | 46.4 |
| L149R + S388K | 47.3 |
| L149R + D11E | 46.4 |
| A364G + D11E | 45.6 |
| S388K + D11E | 45.2 |

### Example 4: Thermostability of Purified Xylanase Variants Nano-Differential Scanning Fluorimetry (DSF)

Melting temperature (Tₘ) of purified xylanase variants was measured using nano-DSF. Purified protein was diluted to 100 ppm using 50 mM sodium acetate at pH 4.0. Sodium acetate buffer (50 mM) was prepared by adding 4.1 g of sodium acetate (Sigma, S2889) in 1 L of Milli-Q water and adjusting the pH to 4.0 using acetic acid. Diluted samples were filled in n-DSF glass capillaries (PR-0002, Nanotemper Technologies) by dipping capillary in the sample and placed in a slot of the n-DSF instrument (Prometheus NT.48, Nanotemper Technologies). Each sample was analyzed in triplicate. Hence, total 16 samples were analyzed in 48 capillaries in a single run. In each run, purified xylanase-WT was used as a control. Samples were scanned from 25-65° C. at a ramp speed of 1.5° C./min. T, was obtained from first-derivative curve plotted from raw data using inbuilt software provided by Nanotemper Technologies.

Tm of the xylanase variant and the Tm of the wild-type xylanase are presented in table 3 below. The results in table 3 shows that the xylanase variants have improved thermostability relative to the wild-type xylanase (SEQ ID NO: 1).

TABLE 3
Thermostability of purified xylanase variants
of SEQ ID NO: 1 using nano-DSF

| | Tm |
|---|---|
| Variant(s) | (° C.) |
| WT | 43.6 |
| V45L + A79C + A117S + L149R + S214Q + V258P + A276S + | 57.8 |
| A364G + S388K + D470T + R483V | |
| V45L + A79C + A117S + L149R + S214Q + V258P + A276S + | 59.1 |
| H358D + A364G + S388K + D470T | |
| V45L + A117S + L149R + S214Q + V258P + A276S + H358D + | 57.2 |
| A364G + S388K + D470T + R483V | |
| V45L + Q61K + A117S + L149R + S214Q + V258P + A276S + | 57.9 |
| H358D + A364G + S388K + D470T | |
| V45L + D95N + A117S + L149R + S214Q + V258P + A276S + | 57.5 |
| H358D + A364G + S388K + G394A + D470T | |
| V45L + A117S + L149R + S214Q + V258P + A276S + A364G + | 56.2 |
| S388K + G394A + D470T + R483V | |
| V45L + A93T + A117S + L149R + S214Q + V258P + A276S + | 56.5 |
| H358D + A364G + S388K + D470T + A499Y | |
| V45L + A117S + L149R + S214Q + V258P + A276S + A364G + | 56.7 |
| Q384A + S388K + D470T + R483V | |
| V45L + A79C + A117S + L149R + S214Q + V258P + A276S + | 58.2 |
| H358D + A364G + S388K | |
| V45L + A117S + L149R + S214Q + V258P + A276S + A364G + | 55.5 |
| S388K + D470T + R483V | |
| V45L + A117S + L149R + S214Q + V258P + A276S + H358D + | 56.9 |
| A364G + S388K + D470T | |
| V45L + A79C + A117S + L149R + S214Q + V258P + A276S + | 57.2 |
| A364G + S388K + D470T | |
| V45L + D95N + A117S + L149R + S214Q + V258P + A276S + | 56.1 |
| H358D + A364G + S388K + S473G | |
| V45L + T70R + D95N + A117S + L149R + S214Q + V258P + | 57.3 |
| A276S + L356M + H358D + A364G + S388K | |
| V45L + A117S + L149R + S214Q + V258P + A276S + H358D + | 56.7 |
| A364G + S388K + D470A | |
| V45L + A117S + L149R + S214Q + V258P + A276S + H358D + | 56.5 |
| A364G + S388K + L478N | |
| V45L + A117S + L149R + S214Q + V258P + A276S + H358D + | 56.3 |
| A364G + S388K + N395D | |
| V45L + A117S + L149R + S214Q + V258P + A276S + H358D + | 56.7 |
| A364G + S388K + T477K | |
| V45L + A117S + L149R + S214Q + V258P + A276S + L281V + | 56.7 |
| H358D + A364G + S388K | |
| D11E + V45L + A117S + L149R + S214Q + V258P + A276S + | 56.5 |
| H358D + A364G + S388K | |
| V45L + L149R + V258P + A276S + H358T + A364G | 52.3 |
| A25W + V45L + L149R + V258P + A276S + A364G | 52.5 |
| V45L + L149R + S214Q + V258P + A276S + A364G + S388K | 54.5 |
| V45L + L149R + V258P + A276S + L281V + A364G | 52.5 |
| V45L + N127D + L149R + V258P + A276S + A364G + S388K | 53.4 |
| V45L + L149R + V258P + A276S + H358T + A364G + S388K | 53.7 |
| V45L + L149R + V258P + A276S + A364G + S388K | 53.3 |
| V45L + L149R + W250Y + V258P + A276S + A364G + S388K | 53.7 |
| V45L + L149R + V258P + A276S + A286E + A364G + S388K | 54.1 |
| V45L + L149R + V258P + A276S + S282N + A364G + S388K | 53.7 |
| V45L + L149R + S214Q + V258P + A276S + H358T + A364G | 53.7 |
| A25W + V45L + L149R + V258P + A276Q + A364G | 52.1 |
| V45L + L149R + V258P + A276S + A364G | 53.1 |
| A25W + V45L + L149R + W250Y + A276Q + A364G | 51.1 |
| A25W + V45L + L149R + A276Q + L281V + A364G | 51.9 |
| A25W + V45L + L149R + A276Q + A286E + A364G | 51.2 |
| A25W + V45L + L149R + A276Q + A364G + S388K | 51.5 |
| V45L + L149R + V258P + L281V + A364G | 51.3 |
| A25W + V45L + L149R + A276Q + A364G | 51.2 |
| V45L + L149R + V258P + A364G | 50.6 |
| V45L + L149R + V258P + S282N + A364G | 50.8 |
| V45L + L149R + A276Q + A364G | 49.3 |
| V45L + L149R + A276S + A364G | 51.0 |
| V45L + L149R | 48.1 |
| V45L + L149R + A364G | 48.5 |
| V45L + L149R + V258R + A364G | 50.4 |
| V258P + A276Q | 48.0 |
| V258P + A276S | 47.9 |
| V45L + A276S | 47.9 |
| V45L + A276Q | 48.1 |
| L149R + W250Y | 46.9 |
| V258P + S388K | 47.1 |
| A276Q + S388K | 47.4 |
| W250Y + A276Q | 47.5 |
| A276S + S388K | 47.5 |
| A276Q + L281V | 47.2 |
| W250Y + A276S | 46.6 |
| A276S + L281V | 47.0 |
| G187S + A276Q | 46.3 |
| V45L + L281V | 45.9 |
| V45L | 45.2 |
| L149R | 45.6 |
| A79C | 46.5 |
| V45N | 45.7 |
| V45L + D11E | 46.1 |
| L149R + S388K | 47.3 |
| L149R + D11E | 46.5 |
| A364G + D11E | 45.2 |
| S388K + D11E | 45.7 |

### Example 5: Thermostability of Purified Xylanase Variants Under High Sulphur Dioxide

In this experiment, xylanase variants and wildtype xylanase were tested for thermostability in presence of high sulphur dioxide.

The thermostability screening assay generally includes an enzyme stress test by incubating enzymes (xylanase variants) in corn fiber extract followed by an activity test incubating the stressed enzyme in fine fiber. In the first stage, enzymes were incubated in fiber extract at application relevant conditions (pH 4.0, temperature 48-52° C.) in an Infors Multitron Pro. The plates were mixed at 600 rpm and 0.5 mL aliquots are pipetted into a 96 Fisherbrand deepwell plate and were stored at 4° C. until further use. The samples were transferred into a Fisher Scientific 24 well plate containing 2.5 mL of 3% dry solids corn fine fiber. Samples were vortexed thoroughly and placed in the Infors Multitron Pro and incubated at 48° C. for 2 hours, 600 rpm.

Upon completion of the secondary incubation, samples weere centrifuged in an Avanti J-E centrifuge at 3,500 rpm for 10 minutes at 10° C. The supernatant was gently pipetted off so as not to disturb the pelleted material. The amount of reducing sugars was determined in the supernatant via the addition of 125 uL of dinitrosalicyclic acid reagent (DNS) to 75 uL of sample. If necessary, samples are diluted using distilled water before the DNS reaction. Samples were incubated for 10 minutes at 95° C. in a C1000 Touch PCR thermocycler from BioRad. The available reducing sugars quantitatively react with the DNS causing an increase in the red-orange spectra (Miller, Analytical Chemistry 1959). The samples were centrifuged at 3,500 rpm, 30 seconds to collect condensate and cooled to ambient temperature. 150 uL of centrifuged sample was then transferred using a multichannel pipette to a Nunc F 96 well plate. Absorbance at 560 nanometers (nm) was read using a Tecan Infinite M1000.

Thermostability assay was run at pH 4.0 for two hours at 52° C., with the addition of 1000 ppm of sulfur dioxide. 500 ppm/mL of xylanase variant and wildtype xylanase enzyme were added into the fiber extract. Samples were pulled as soon as the addition of enzymes to fiber extract was completed, to be considered 100% active baseline. After two hours of incubation, all samples were added into corn fine fiber with a final concentration of 100 ppm/mL. The fine fiber was incubated for two hours, 48° C., 600 rpm. After incubation, the reducing ends were determined by absorbance at 560 nm. All treatments were run in triplicate. Absorbance values and retained activity from fiber are reported in Table 4 with ±standard deviation, n=3. The xylanase variants showed higher thermostability (increased retained activity) when compared to wildtype xylanase (SEQ ID NO: 1).

TABLE 4
Results of residual activities

| | Average | Average | Retained |
|---|---|---|---|
| | Absorption, | Absorption, | Activity |
| | 0 hours | 2 hours | (% of |
| Treatment | (560 nm) | (560 nm) | original) |
| WT (SEQ ID NO: 1) | 0.900 (0.065) | 0.138 (0.045) | 15% |
| V45L + T70R + A117S + L149R + | 0.904 (0.125) | 0.717 (0.056) | 79% |
| S214Q + V258P + A276S + L281V + | | | |
| H358D + A364G + S388K + G394A + | | | |
| D470T + R483V | | | |
| V45L + A79C + A117S + L149R + | 0.912 (0.139) | 0.466 (0.196) | 51% |
| S214Q + V258P + A276S + A364G + | | | |
| S388K + D470T + R483V | | | |
| V45L + A117S + L149R + S214Q + | 0.889 (0.095) | 0.284 (0.237) | 32% |
| V258P + A276S + H358D + A364G + | | | |
| S388K + D470T + R483V | | | |

### Example 6

### Method Description Fiber Washing Assay

The assay includes incubating wet fiber samples obtained from wet-milling plant, in the presence of enzymes, at conditions relevant to the process (pH 4, Temp ˜50° C.) and over a time period of between 1 to 4 hr. After incubation, the fiber is transferred and pressed over a screen (typically 100 micron or smaller), through which the filtrates consisting mainly of the separated starch and gluten are then collected. A number of washes are done over the screen, and the washings are collected together with the initial filtrate. The collected filtrate was then passed over a funnel filter (glass filter with 0.45 micron opening) to further separate the insoluble solids (starch and gluten) from the rest of the filtrates (mostly dissolved solids). These retained insoluble solids are washed and then oven dried to dryness. The insoluble dry mass is weighed and then analyzed for starch content

In this experiment, xylanase variants and wildtype xylanase were tested for product and performance in terms of total insoluble solid yields and starch yields. Wet-mill fiber with 8% dry solids content were treated with total of 300 pg enzyme protein per gram dry solid substrate, of which 10% (30 pg/g ds) is xylanase, and the remaining 90% consisted of the background cellulases. Cellulases are all cellulase enzymes produced by fermentation of Trichoderma reesei. The gram scale fibewashing assay was done with 2 hr incubation, at pH 4.0 and 50° C. The results are reported in Table 5 below. The xylanase variants showed superior performance when compared to wildtype xylanase (SEQ ID NO: 1).

TABLE 5
Results of residual activities

| | Insolubles | |
|---|---|---|
| | Yield (% w/w | Starch Yield |
| | of starting | (% w/w of |
| GH5_21 Component | fiber dry | starting fiber |
| (10% unless indicated) | solids) | dry solids) |
| WT (SEQ ID NO: 1) | 33.2% (1.8%) | 26.0% (2.1%) |
| V45L + A117S + L149R + S214Q + | 35.7% (0.5%) | 28.4% (1.5%) |
| V258P + A276S + H358D + A364G + | | |
| S388K + D470T + R483V | | |
| V45L + A79C + A117S + L149R + | 38.1% (1.5%) | 30.1% (1.2%) |
| S214Q + V258P + A276S + A364G + | | |
| S388K + D470T + R483V | | |
| V45L + T70R + A117S + L149R + | 37.9% (0.7%) | 29.9% (0.7%) |
| S214Q + V258P + A276S + L281V + | | |
| H358D + A364G + S388K + G394A + | | |
| D470T + R483V | | |

### Example 7

The 10-g fiber assay uses a substrate obtained from corn fiber samples from a wet mill, which is incubated in the presence of enzymes, at conditions relevant to the process (pH 4, temperature around 50° C.) and over a time period of between 1 to 4 hrs. After incubation, the fiber was transferred and pressed over a 75-micron screen where the filtrates consisting mainly of the separated starch and gluten are then collected. A number of washes are done over the screen, and the washed materials passing through the screen are collected together with the initial filtrate. The collected filtrates are allowed to sit overnight letting the insoluble settle to the bottom of the flask. The bulk of the supernatant was aspirated via vacuum and the rest of the insolubles are then centrifuged in 50 ml conical tubes (3000 rpm for 10 min), after which the supernatant was decanted leaving a wet insoluble pellet. The wet insoluble pellet was lyophilized overnight to complete dryness. This insoluble dry mass was weighed to determine % insoluble yield (calculated on the basis of the starting fiber substrate weight), and in some cases analyzed for starch content to determine the % starch yield.

In this experiment, fiber containing 11.6% moisture were weighed into flasks to a weight of 5-gram dry solids, then added with pH 4 Na acetate buffer (˜40 mM) to a final slurry containing 8% dry solids. Wet-mill fiber with 8% dry solids content were treated with total of enzymes at a dose of 300 micrograms of protein per gram of fiber dry solids. A set (of triplicates) did not receive any enzyme treatment and was incubated only in buffer (control). All others received the experimental blend containing 270 micrograms total protein produced from a Trichoderma reesei (this protein blend comprises all naturally produced cellulase enzymes produced by fermentation of Trichoderma reese;) fermentation (The cellulase blend composition will comprise all cellulase activities expressed in T. reesei; e.g., endoglucanases, and cellobiohydrolases. Alternatively, a commercial blend is available from Sigma Aldrich, Celluclast 1.5) plus 30 micrograms of the GH5_21 xylanase (the wild type or the variants). Table 6 show the amounts of insoluble solids yields (expressed as percentages of the starting fiber weight) from treatments with the different variant and control. The results are reported in Table 6 below. The xylanase variants showed superior performance when compared to wildtype xylanase (SEQ ID NO: 1).

TABLE 6
RESULTS OF INSOLUBLE SOLIDS YIELDS

| | Mean % | |
|---|---|---|
| | Insoluble | Difference |
| GH5_21 Component | Solids | (Treatment- |
| (10% unless indicated) | Yield | Control) |
| Control (No enzyme) | 29.5% | 0 |
| WT (SEQ ID NO: 1) | 33.2% | 3.7% |
| V45L + T70R + A117S + L149R + | 37.9% | 8.5% * |
| S214Q + V258P + A276S + L281V + | | |
| H358D + A364G + S388K + G394A + | | |
| D470T + R483V | | |
| V45L + A117S + L149R + S214Q + | 38.1% | 8.7% * |
| V258P + A276S + H358D + A364G + | | |
| S388K + D470T + R483V | | |
| V45L + A79C + A117S + L149R + | 35.7% | 6.3% * |
| S214Q + V258P + A276S + A364G + | | |
| S388K + D470T + R483V | | |

Standard error of mean = 1.3% (ANOVA, n = 3)
* p-Value < 0.05 using Dunnett's method

### Example 8

In this experiment, fiber containing 37.6% moisture were weighed into flasks to a weight of 5-gram dry solids, then added with pH 4 Na acetate buffer (˜40 mM) to a final slurry containing 8% dry solids. The fiber slurry were all dosed with 450 micrograms of total protein produced from a Trichoderma reesei this protein blend comprises all naturally produced cellulase enzymes produced by fermentation of Trichoderma reese;) fermentation (The cellulase blend composition will comprise all cellulase activities expressed in T. reesei; e.g., endoglucanases, and cellobiohydrolases. Alternatively, a commercial blend is available from Sigma Aldrich, Celluclast 1.5) for each gram of dry solids weight fiber. Each experimental set (of triplicates) was then added with 50 micrograms (per gram dry solid weight fiber) of the GH5_21 xylanase (wildtype or variants). One set received a higher dose of 300 micrograms of one of the GH5_21 variants to confirm a dose response. Table 7 show the amounts of starch yields (expressed as percentages of the starting fiber weight) from treatments with the different variant and control. The results are reported in Table 7 below.

TABLE 7
RESULTS OF STARCH YIELDS

| | Mean % | Difference |
|---|---|---|
| GH5_21 Component | Starch | (Variant- |
| (10% unless indicated) | Yield | WT) |
| WT (SEQ ID NO: 1) | 12.2% | 0 |
| A25W + V45L + D95N + L149R + | 12.6% | 0.4% |
| A276Q + A364G + S388K | | |
| A25W + V45L + L149R + V258P + | 12.7% | 0.5%* |
| A276Q + A364G | | |
| V45L + L149R + V258P + A276S + | 12.8% | 0.7%* |
| L281V + A364G | | |
| V45L + L149R + V258P + L281V + | 12.5% | 0.4% |
| A364G | | |
| V45L + L149R + V258P + L281V + | 13.4% | 1.3%* |
| A364G (high dose) | | |

Standard error of mean = 1.3% (ANOVA, n = 3)
*p-Value < 0.05 using Dunnett's method

### Example 9

In this experiment, fiber containing 38.4% moisture were weighed into flasks to a weight of 10-gram dry solids, then added with pH 4 Na acetate buffer (˜40 mM) to a final slurry containing 8% dry solids. The fiber slurry were all dosed with 400 micrograms of total protein produced from a Trichoderma reesei (this protein blend comprises all naturally produced cellulase enzymes produced by fermentation of Trichoderma reesei) fermentation (The cellulase blend composition will comprise all cellulase activities expressed in T. reesei; e.g., endoglucanases, and cellobiohydrolases. Alternatively, a commercial blend is available from Sigma Aldrich, Celluclast 1.5) for each gram of dry solids weight fiber. Each experimental set (of triplicates) was then added with 25 micrograms (per gram dry solid weight fiber) of the GH5_21 xylanase, wildtype or variants. Table 8 show the amounts of insoluble solids yields (expressed as percentages of the starting fiber weight) from treatments with the different variant and control. The results are reported in Table 8 below.

TABLE 8
RESULTS OF INSOLUBLE SOLIDS YIELDS

| GH5_21 Component | Mean % Insoluble | Difference |
|---|---|---|
| (10% unless indicated) | Solids Yield | (Variant-WT) |
| WT (SEQ ID NO: 1) | 25.6% | 0 |
| V45L + L149R | 26.9% | 1.3%* |
| V45L + A79C + L149R + A364G | 26.1% | 0.5% |
| V45L + L149R + A364G | 26.0% | 0.5% |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A xylanase variant of a parent xylanase comprising a substitution at one or more positions corresponding to position 45, 149, 364, 258, 276, and 388 of SEQ ID NO: 1 and wherein said variant has xylanase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to the polypeptide of SEQ ID NO: 1.

2. The xylanase variant according to claim 1, which has increased thermostability compared to the xylanase of SEQ ID NO: 1.

3. The xylanase variant according to claim 1, wherein said increased thermostability is measured as increased melting temperature using TSA of at least 0.5° C., at least 1° C., at least 1.5° C., at least 2° C., at least 2.5° C., at least 3° C., at least 3.5° C., at least 4.0° C., at least 4.5° C. or at least 5° C.

4. The xylanase variant according to claim 1 further comprising a substitution at one or more positions corresponding to position to 9, 11, 18, 19, 20, 24, 25, 28, 40, 60, 61, 69, 70, 77, 79, 92, 93, 95, 113, 117, 127, 161, 163, 168, 170, 173, 180, 187, 190, 192, 211, 214, 215, 219, 234, 243, 250, 252, 260, 281, 282, 286, 314, 334, 340, 350, 355, 356, 358, 366, 375, 384, 385, 386, 389, 391, 393, 394, 395, 396, 446, 459, 468, 469, 470, 471, 472, 473, 474, 476, 477, 478, 480, 481, 483, 484, 495, 496, 497, 498, 499, 500, 501, 503, 522, 525, 526, 527, 528, 529, 530, and 540 of SEQ ID NO: 1.

5. The xylanase variant according to claim 4, wherein said variant comprises one or more of the following substitutions at a position corresponding to positions D9, D11, A18, S19, A20, A24, A25, M28, C40, V45, C60, Q61, C69, T70, Q77, A79, S92, A93, D95, D113, A117, N127, L149, C161, N163, N168, A170, S173, T180, G187, S190, D192, T211, S214, T215, H219, V234, W250, A252, V258, N260, A276, L281, S282, A286, Q314, H334, A340, V350, N355, L356, H358, A364, T366, C375, Q384, Q385, Y386, S388, S389, Y391, V393, G394, N395, C396, D446, R459, C468, I469, D470, L471, A472, A472, S473, S473, N474, N476, T477, L478, T480, S481, R483, L484, W495, Q496, V497, V498, A499, V500, S501, G503, C522, D525, G526, N527, S528, N529, L530 and C540 of SEQ ID NO: 1.

6. The xylanase variant according to claim 5, wherein said variant comprises one or more of the following substitutions at a position corresponding to positions D9E, D11E, A18V, 519T, A20H, A20P, A24H, A24K, A25W, M28P, C40A, C40L, C40S, C40V, C60A, C60L, C60S, C60V, Q61K, Q61R, C69A, C69L, C69S, C69V, T70R, Q77E, A79C, S92D, S92N, A93S, A93T, D95N, D113T, A117C, A117S, N127D, C161L, C161S, C161V, N163H, N168G, A170S, S173Y, T180Q, G187K, G187L, G187S, S190E, D192E, T211H, S214E, S214Q, S214Y, T215I, T215K, T215V, H219I, H219V, V234P, C243L, C243S, C243V, W250Y, A252S, N260H, L281V, S282N, A286E, A286S, Q314A, H334D, A340G, V350L, N355G, L356M, H358D, H358T, T366I, T366V, C375A, C375L, C375S, C375V, Q384A, Q385C, Y386G, S389H, S389V Y391V, V393H, V393R, G394A, G394D, G394Q, N395D, C396A, C396L, C396A, C396S, C396V, D446T, R459V, 0468A, C468L, C468S, C468V, 1469T, D470A, D470K, D470T, D470V, L471R, A472I, A472L, S473G, S473K, N474G, N476D, N476N, T477K, T477V, L478N, L478P, T480L, T480N, S481A, R483V, L484D, L484K, L484S, W495E, W495I, W495S, Q496E, Q496W, Q496R, V497D, V497E, V497P, V497W, V498I, A499L, A499Y, V500T, S501E, 5501N, G503L, C522A, C522S, C522V, D525L, G526T, N527H, S528H, N529T, L530R, C540A, C540L, C540S, and C540V of SEQ ID NO: 1.

7. A method of producing a xylanase variant according to claim 1, comprising cultivating the host cell under conditions conducive for production of the polypeptide and further comprising recovering the polypeptide.

8. A method of obtaining a xylanase variant, comprising (a) introducing into a parent xylanase a substitution at one or more positions corresponding to positions 45, 149, 364, 258, 276, and 388 of SEQ ID NO: 1, wherein the xylanase variant has xylanase activity and has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to SEQ ID NO: 1; and (b) optionally recovering the xylanase variant.

9. A composition comprising the xylanase variant of claims-1.

10. A method for increasing the starch yield and/or gluten yield from corn kernels in a wet milling process, the method comprising contacting corn kernels or a fraction of the corn kernels, particularly a fiber rich fraction, with an effective amount of xylanase variant of claim 1 and optionally one or more hydrolytic enzymes.

11. The method according to claim 10, comprising the steps of:
a) soaking the corn kernels in water to produce soaked kernels;
b) grinding the soaked kernels to produce ground kernels;
c) separating germ from the ground kernels to produce a corn kernel mass comprising fiber, starch and gluten; and
d) subjecting the corn kernel mass to a fiber washing procedure separating starch and gluten from the fiber;
wherein at least a xylanase variant of claim 1 is present/added before or during step d).

12. The method of claim 11, further comprising the steps of:
e) separating the starch from the gluten; and optionally
f) washing the starch.

13. The method according to claim 10, wherein the one or more hydrolytic enzymes comprises one or more cellulases.

14. The method according to claim 13, wherein the cellulase(s) comprises one or more enzyme selected from the group consisting of an endoglucanase (EG), and a cellobiohydrolase (CBH).

15. (canceled)
